(19) United States
(12) Patent Application Publication
(10) Pub. No.: US 2023/0242498 A1
(43) Pub. Date: Aug. 3, 2023

(54) NOVEL COMPOUND AND ORGANIC LIGHT EMITTING DEVICE COMPRISING THE SAME

(71) Applicant: LG CHEM, LTD., Seoul (KR)

(72) Inventors: Minjun KIM, Daejeon (KR); Sang Duk SUH, Daejeon (KR); Young Seok KIM, Daejeon (KR); Dong Hoon LEE, Daejeon (KR)

(21) Appl. No.: 17/924,464

(22) PCT Filed: Aug. 19, 2021
(86) PCT No.: PCT/KR2021/011030

§ 371 Date: Nov. 10, 2022

(30) Foreign Application Priority Data
Aug. 19, 2020 (KR) 10-2020-0104200
Aug. 19, 2021 (KR) 10-2021-0109437

(51) Int. Cl. C07D 307/91; H10K 50/11; H10K 85/60; C07D 333/76

(52) U.S. Cl. CPC C07D 307/91; H10K 50/11; H10K 85/615; H10K 85/6574; H10K 85/636; H10K 85/626; C07D 333/76; H10K 85/6576

(57) ABSTRACT

Provided is a compound of Chemical Formula 1:

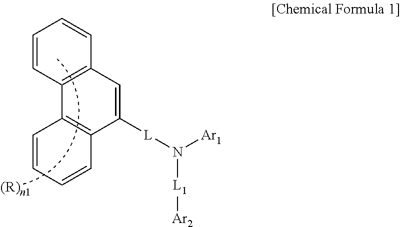

wherein in Chemical Formula 1: L is a substituted or unsubstituted C₆₋₆₀ arylene; L₁ is a single bond or a substituted or unsubstituted C₆₋₆₀ arylene; Ar₁ is any one substituent of the following:

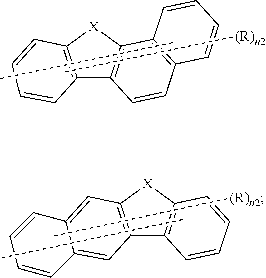

X is O or S; Ar₂ is a substituted or unsubstituted C₆₋₆₀ aryl; each R is independently hydrogen or deuterium; n1 is an integer of 0 to 9; and n2 is an integer of 0 to 9, and an organic light emitting device including the same.

### CROSS-REFERENCE TO RELATED APPLICATION(S)

This application is a National Stage Application of International Application No. PCT/KR2021/011030 filed on Aug. 19, 2021, which claims priority to and the benefit of Korean Patent Application No. 10-2020-0104200 filed on Aug. 19, 2020 and Korean Patent Application No. 10-2021-0109437 filed on Aug. 19, 2021 in the Korean Intellectual Property Office, the contents of which are incorporated herein by reference in their entirety.

### TECHNICAL FIELD

The present disclosure relates to a novel compound and an organic light emitting device comprising the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuous need to develop a new material for the organic material used in the organic light emitting device as described above.

### PRIOR ART LITERATURE

### Patent Literature

(Patent Literature 1) Korean Unexamined Patent Publication No. 10-2000-0051826

### BRIEF DESCRIPTION

### Technical Problem

It is an object of the present disclosure to provide a novel compound and an organic light emitting device comprising the same.

### Technical Solution

According to an aspect of the present disclosure, provided is a compound of Chemical Formula 1:

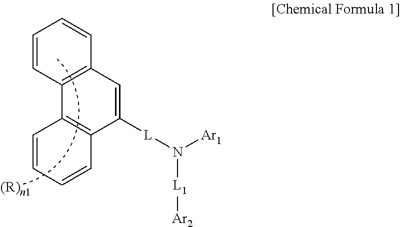

wherein in Chemical Formula 1:

L is a substituted or unsubstituted C₆₋₆₀ arylene;

L₁ is a single bond or a substituted or unsubstituted C₆₋₆₀ arylene;

Ar₁ is any one substituent of the following:

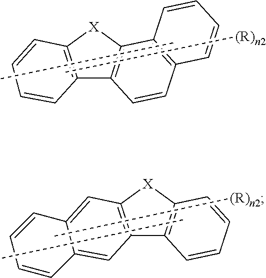

X is O or S;

Ar₂ is a substituted or unsubstituted C₆₋₆₀ aryl;

each R is independently hydrogen or deuterium;

n1 is an integer of 0 to 9; and

n2 is an integer of 0 to 9.

According to another aspect of the present disclosure, provided is an organic light emitting device comprising: a first electrode; a second electrode that is provided opposite to the first electrode; and one or more organic material layers that are provided between the first electrode and the second electrode, wherein one or more layers of the organic material layers comprises the compound of Chemical Formula 1.

### Advantageous Effects

The above-mentioned compound of Chemical Formula 1 can be used as a material of an organic material layer in an organic light emitting device, and can improve the efficiency, achieve low driving voltage and/or improve lifetime characteristics in the organic light emitting device. In particular, the compound of the Chemical Formula 1 can be used as a hole injection material, hole transport material, hole injection and transport material, light emitting material, electron transport material, or electron injection material.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, a light emitting layer 7, an electron transport layer 8, and a cathode 4.

### DETAILED DESCRIPTION

Hereinafter, embodiments of the present disclosure will be described in more detail to facilitate understanding of the invention.

As used herein, the notation

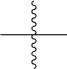

and [symbol] mean a bond linked to another substituent group.

As used herein, the term “substituted or unsubstituted” means being unsubstituted or substituted with one or more substituents selected from the group consisting of deuterium, a halogen group, a nitrile group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heterocyclic group containing at least one of N, O and S atoms, or being unsubstituted or substituted with a substituent to which two or more substituents of the above-exemplified substituents are linked. For example, “a substituent in which two or more substituents are linked” can be a biphenyl group. Namely, a biphenyl group can be an aryl group, or it can also be interpreted as a substituent in which two phenyl groups are linked.

In the present disclosure, the carbon number of a carbonyl group is not particularly limited, but is preferably 1 to 40. Specifically, it can be a compound having the following structure, but is not limited thereto:

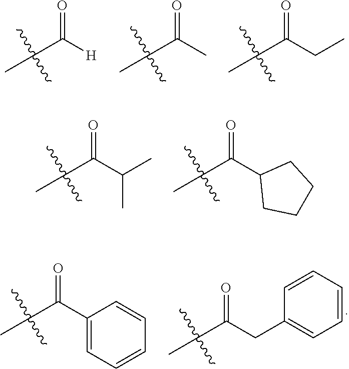

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, it can be a compound having the following structure, but is not limited thereto:

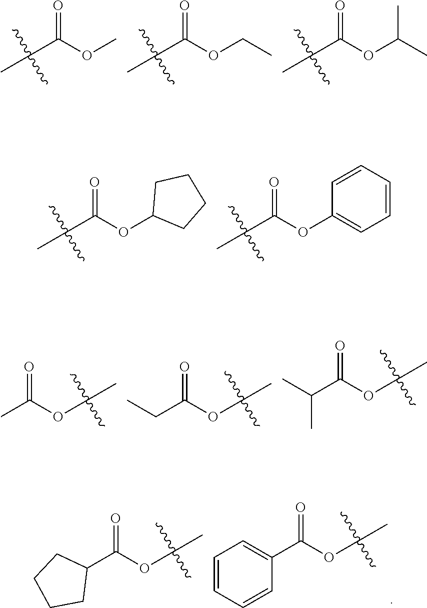

In the present disclosure, the carbon number of an imide group is not particularly limited, but is preferably 1 to 25. Specifically, it can be a compound having the following structure, but is not limited thereto:

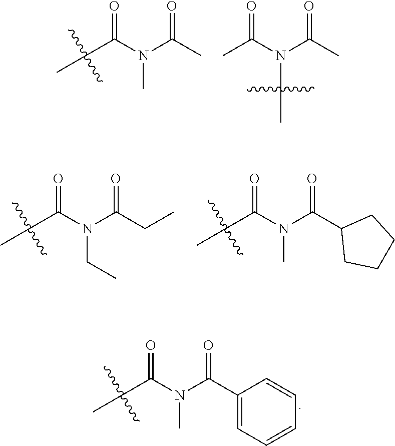

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present disclosure, a boron group specifically includes a trimethylboron group, a triethylboron group, a t-butyldimethylboron group, a triphenylboron group, a phenylboron group, and the like, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cycloheptylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The aryl group can be a phenyl group, a biphenylyl group, a terphenylyl group or the like as the monocyclic aryl group, but is not limited thereto. The polycyclic aryl group includes a naphthyl group, an anthracenyl group, a phenanthryl group, a pyrenyl group, a perylenyl group, a chrysenyl group, a fluorenyl group, or the like, but is not limited thereto.

In the present disclosure, the fluorenyl group can be substituted, and two substituents can be linked with each other to form a spiro structure. In the case where the fluorenyl group is substituted,

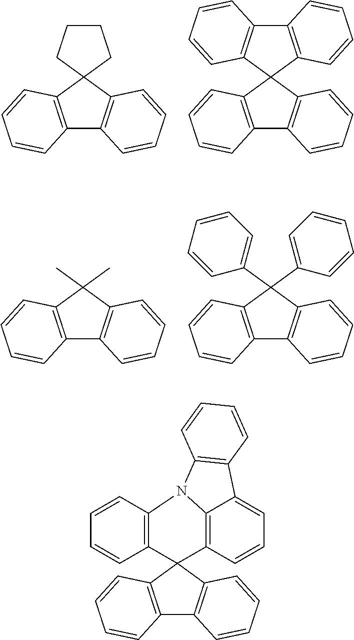

and the like can be formed.

In the present disclosure, a heterocyclic group is a heterocyclic group containing at least one of O, N, Si and S as a heteroatom, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the above-mentioned examples of the aryl group. In the present disclosure, the alkyl group in the aralkyl group, the alkylaryl group and the alkylamine group is the same as the above-mentioned examples of the alkyl group. In the present disclosure, the heteroaryl in the heteroarylamine can be applied to the above-mentioned description of the heterocyclic group. In the present disclosure, the alkenyl group in the aralkenyl group is the same as the above-mentioned examples of the alkenyl group. In the present disclosure, the above-mentioned description of the aryl group can be applied except that the arylene is a divalent group. In the present disclosure, the above-mentioned description of the heterocyclic group can be applied except that the heteroarylene is a divalent group. In the present disclosure, the above-mentioned description of the aryl group or cycloalkyl group can be applied except that the hydrocarbon ring is not a monovalent group but formed by combining two substituent groups. In the present disclosure, the above-mentioned description of the heterocyclic group can be applied except that the heterocyclic group is not a monovalent group but formed by combining two substituent groups.

In Chemical Formula 1, at least one hydrogen can be substituted with deuterium.

Preferably, L is an unsubstituted C₆₋₁₂ arylene. Preferably, L is phenylene, biphenyldiyl, or naphthylene. More preferably, L is any one selected from the group consisting of:

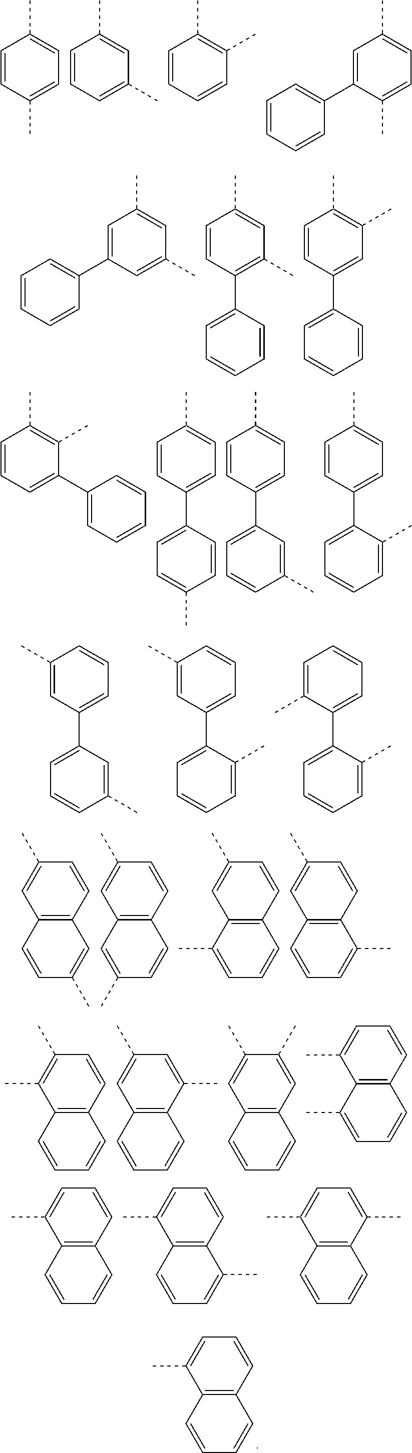

Preferably, L₁ is a single bond, or an unsubstituted C₆₋₁₂ arylene. Preferably, L₁ is a single bond, phenylene, biphenyldiyl, or naphthylene. More preferably, L₁ is a single bond, or any one selected from the group consisting of:

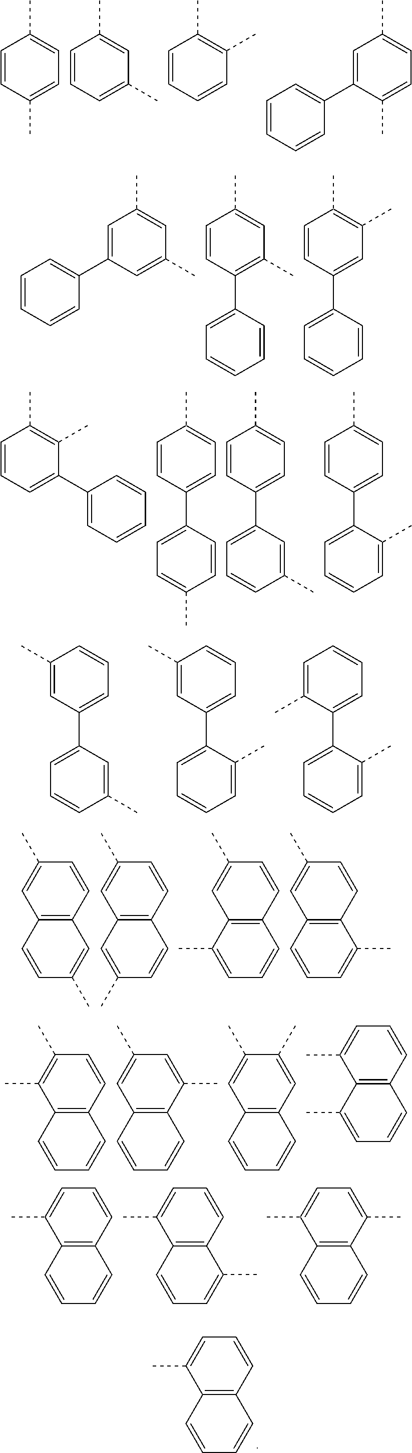

Preferably, Ar₂ is an unsubstituted C₆₋₁₈ aryl. Preferably, Ar₂ is phenyl, biphenylyl, terphenylyl, naphthyl, phenylnaphthyl, naphthylphenyl, phenanthrenyl, or triphenylenyl.

Representative examples of the compound of Chemical Formula 1 are as follows:

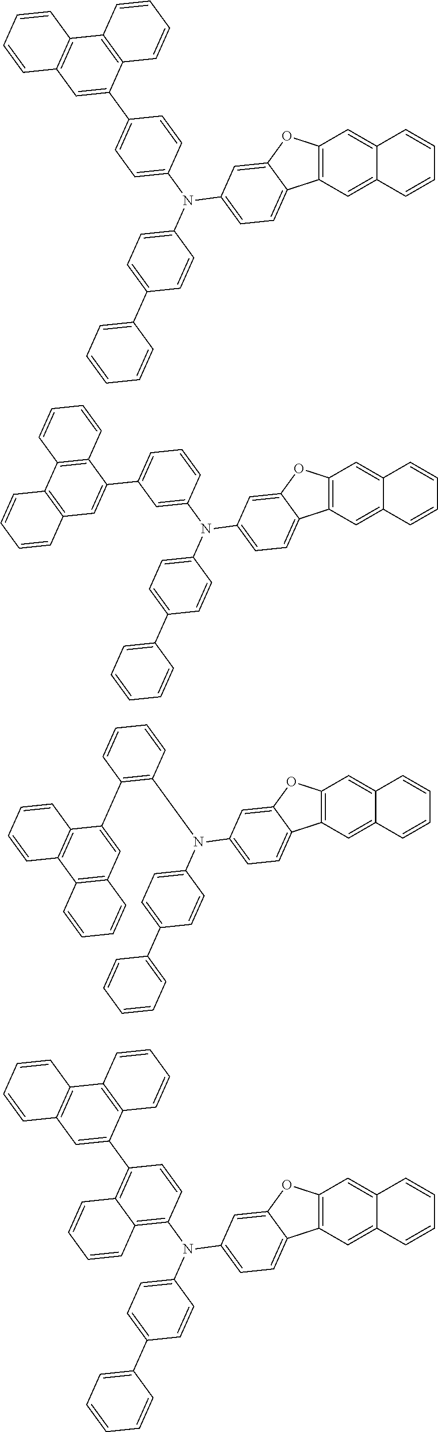

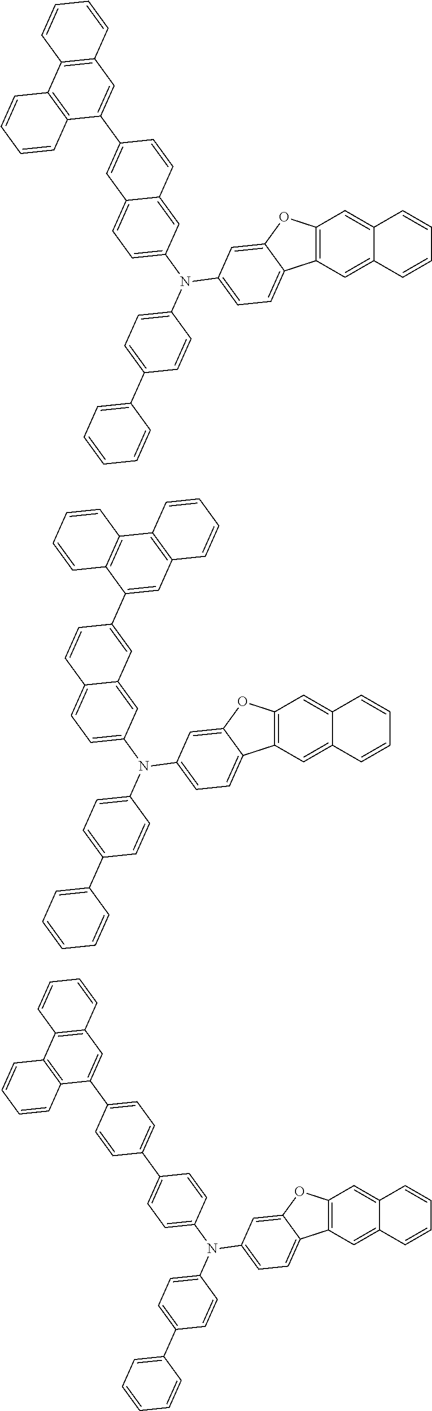

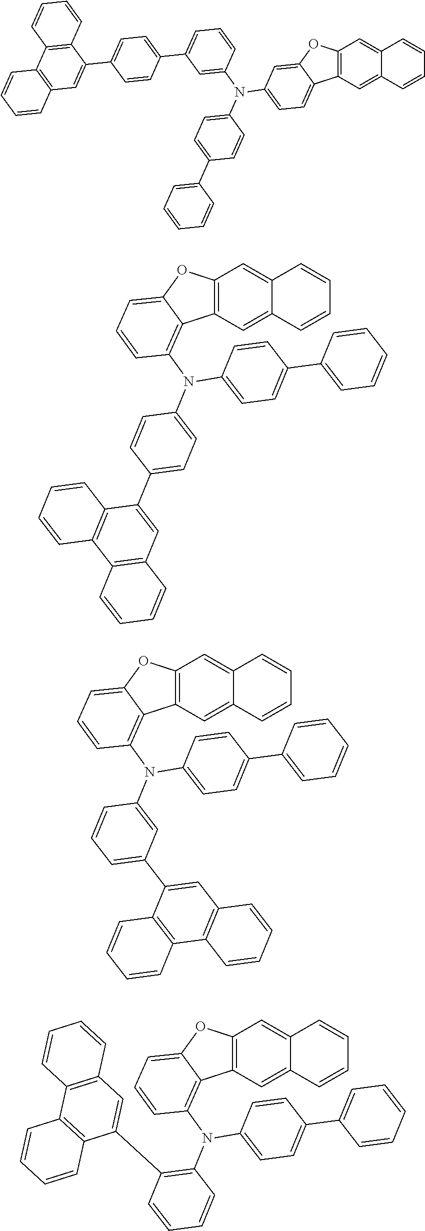

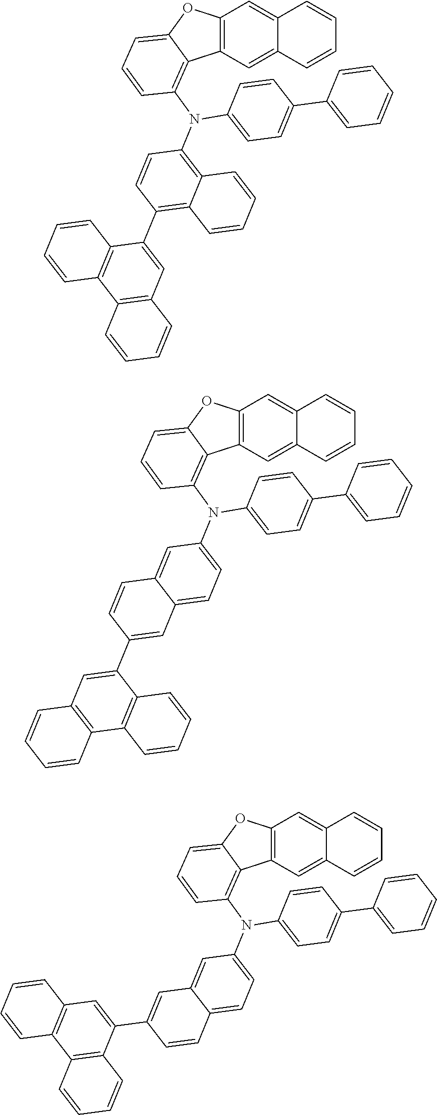 [SMILES: c1ccc(-c2ccc(N(c3ccc(-c4cc5ccccc5c5ccccc45)c4ccccc34)c3cccc4oc5cc6ccccc6cc5c34)cc2)cc1 ; c1ccc(-c2ccc(N(c3ccc4cc(-c5cc6ccccc6c6ccccc56)ccc4c3)c3cccc4oc5cc6ccccc6cc5c34)cc2)cc1 ; c1ccc(-c2ccc(N(c3ccc4ccc(-c5cc6ccccc6c6ccccc56)cc4c3)c3cccc4oc5cc6ccccc6cc5c34)cc2)cc1]

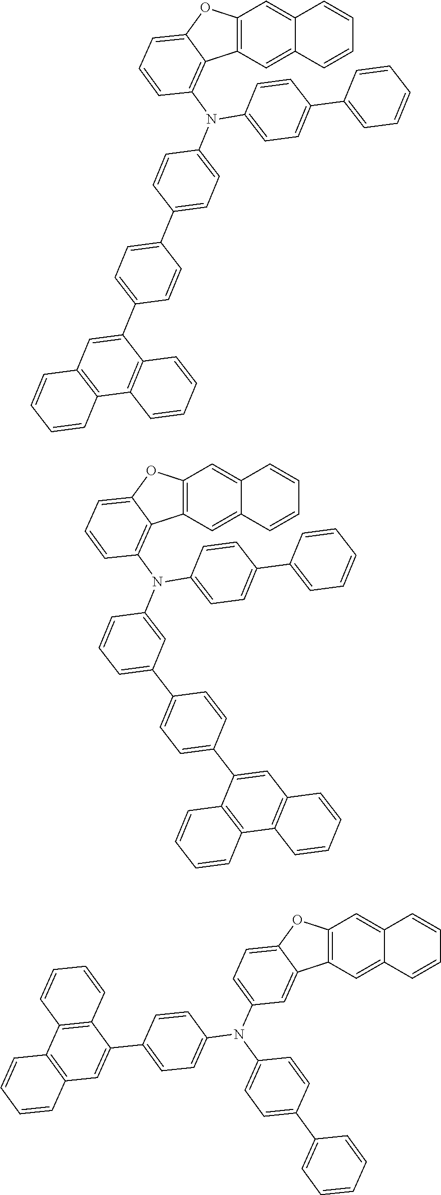 [SMILES: c1ccc(-c2ccc(N(c3ccc(-c4ccc(-c5cc6ccccc6c6ccccc56)cc4)cc3)c3cccc4oc5cc6ccccc6cc5c34)cc2)cc1 ; c1ccc(-c2ccc(N(c3cccc(-c4ccc(-c5cc6ccccc6c6ccccc56)cc4)c3)c3cccc4oc5cc6ccccc6cc5c34)cc2)cc1 ; c1ccc(-c2ccc(N(c3ccc(-c4cc5ccccc5c5ccccc45)cc3)c3ccc4oc5cc6ccccc6cc5c4c3)cc2)cc1]

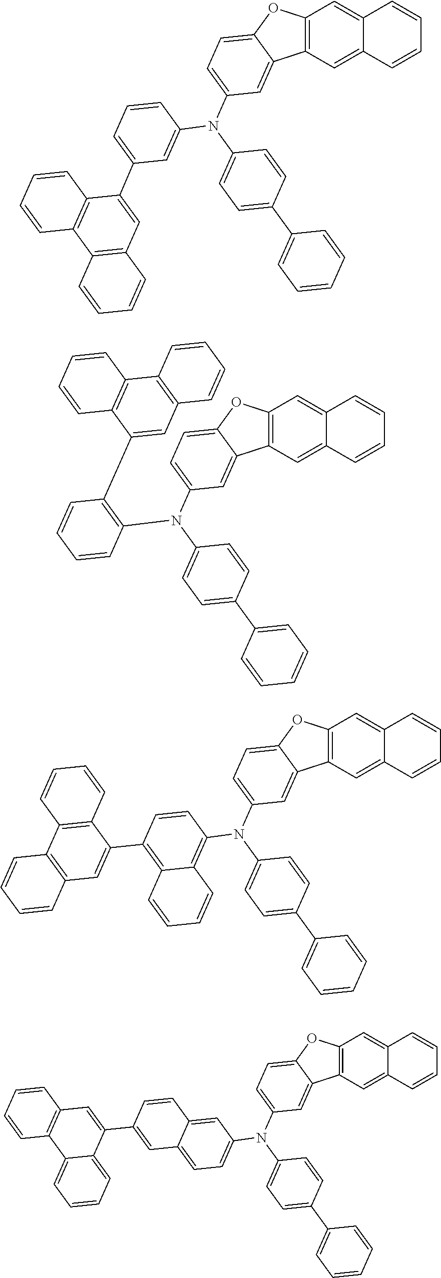

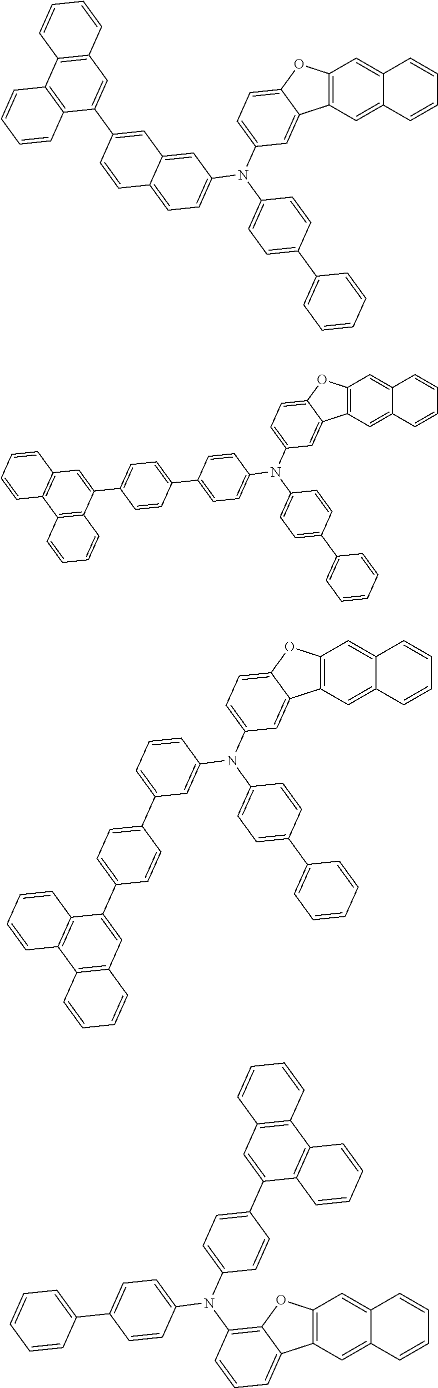

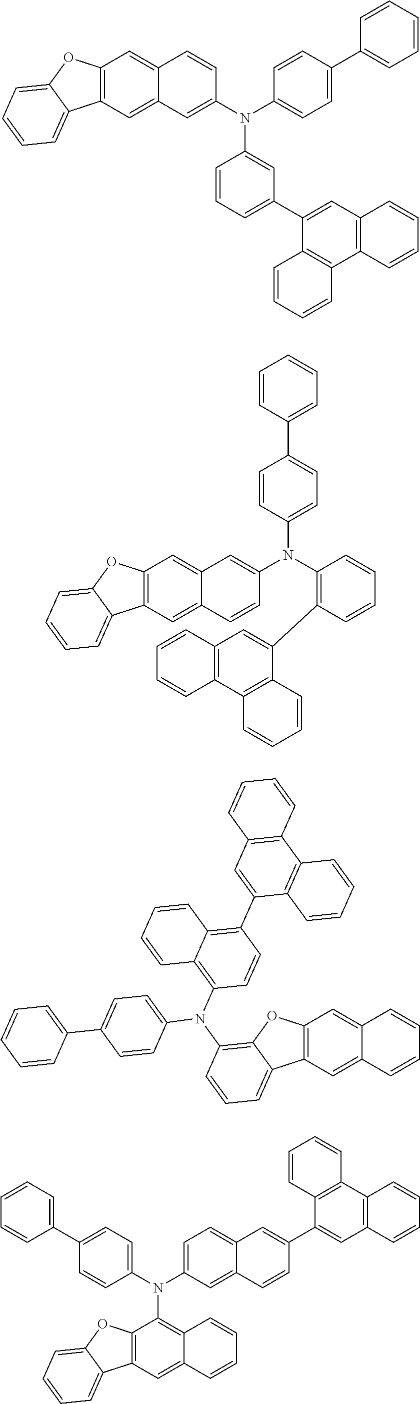

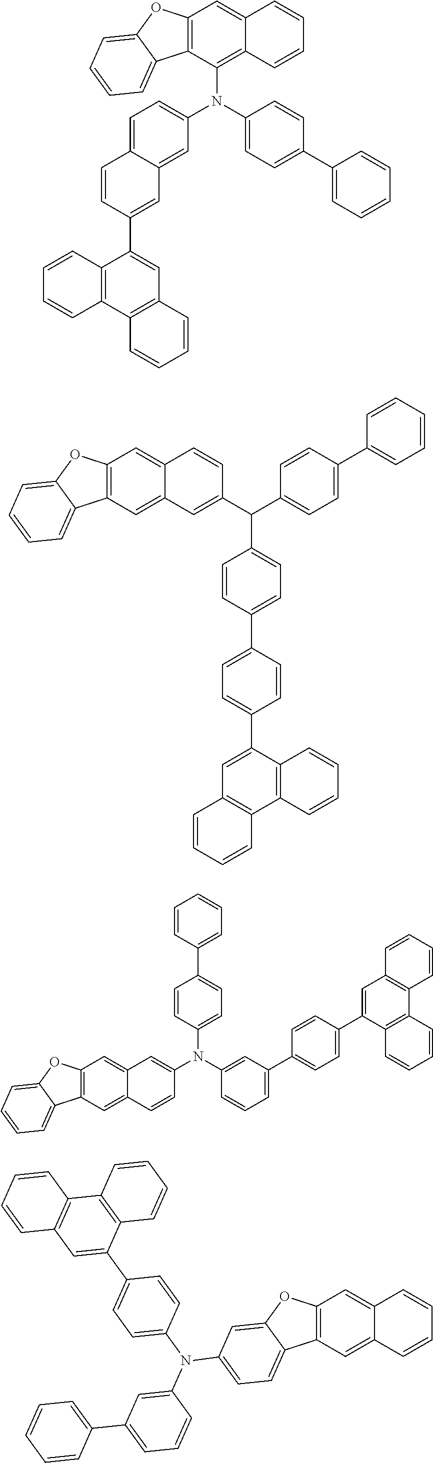

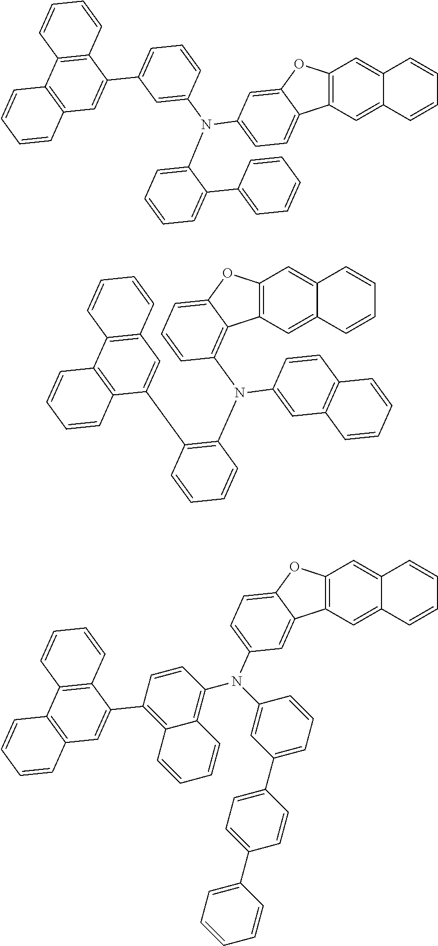 [SMILES: c1ccc(-c2ccccc2N(c2cccc(-c3cc4ccccc4c4ccccc34)c2)c2ccc3c(c2)oc2cc4ccccc4cc23)cc1 ; c1ccc(N(c2ccc3ccccc3c2)c2cccc3oc4cc5ccccc5cc4c23)c(-c2cc3ccccc3c3ccccc23)c1 ; c1ccc(-c2ccc(-c3cccc(N(c4ccc5oc6cc7ccccc7cc6c5c4)c4ccc(-c5cc6ccccc6c6ccccc56)c5ccccc45)c3)cc2)cc1]

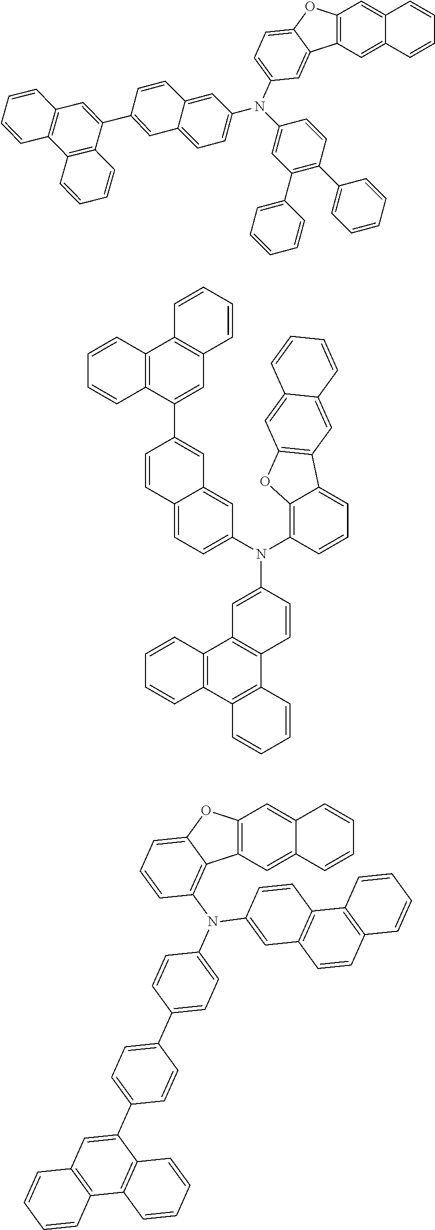 [SMILES: c1ccc(-c2ccc(N(c3ccc4cc(-c5cc6ccccc6c6ccccc56)ccc4c3)c3ccc4oc5cc6ccccc6cc5c4c3)cc2-c2ccccc2)cc1 ; c1ccc2cc3c(cc2c1)oc1c(N(c2ccc4ccc(-c5cc6ccccc6c6ccccc56)cc4c2)c2ccc4c5ccccc5c5ccccc5c4c2)cccc13 ; c1ccc2cc3c(cc2c1)oc1cccc(N(c2ccc(-c4ccc(-c5cc6ccccc6c6ccccc56)cc4)cc2)c2ccc4c(ccc5ccccc54)c2)c13]

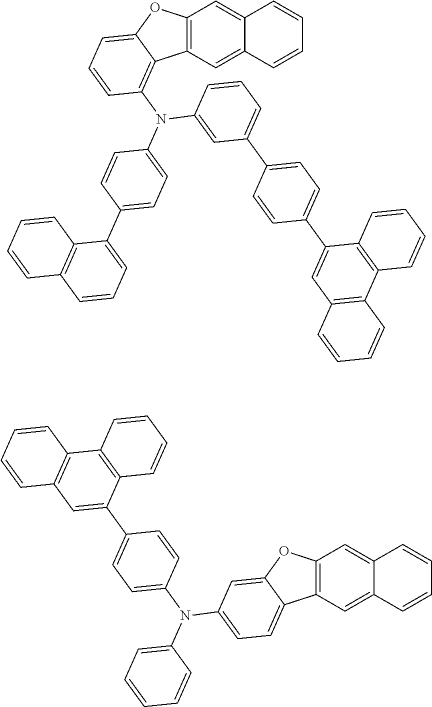 [SMILES: c1cc(-c2ccc(-c3cc4ccccc4c4ccccc34)cc2)cc(N(c2ccc(-c3cccc4ccccc34)cc2)c2cccc3oc4cc5ccccc5cc4c23)c1 ; c1ccc(N(c2ccc(-c3cc4ccccc4c4ccccc34)cc2)c2ccc3c(c2)oc2cc4ccccc4cc23)cc1]

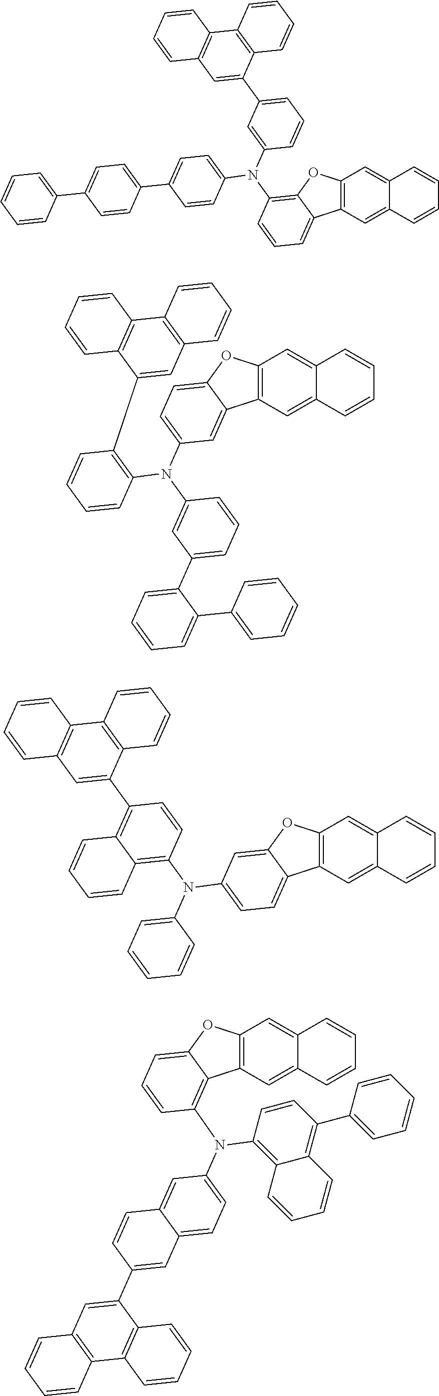

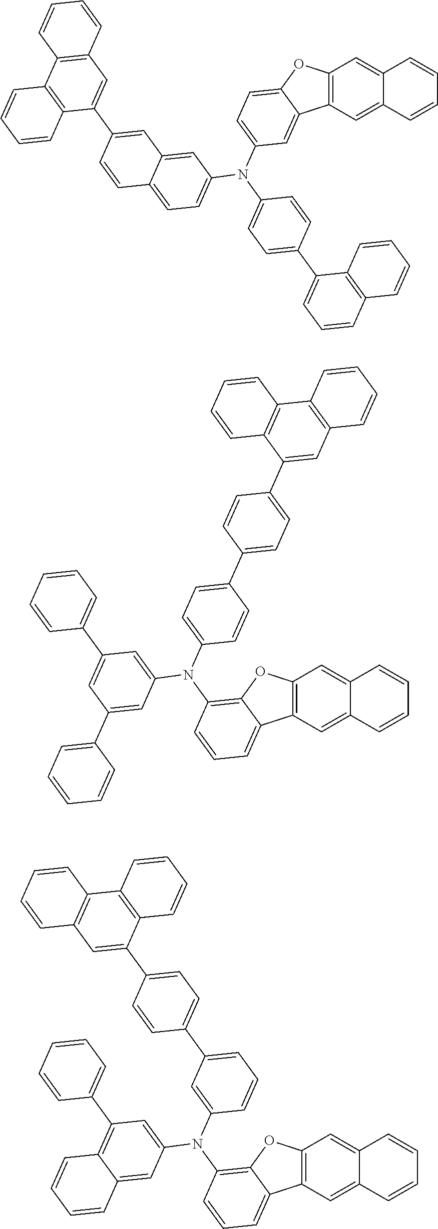

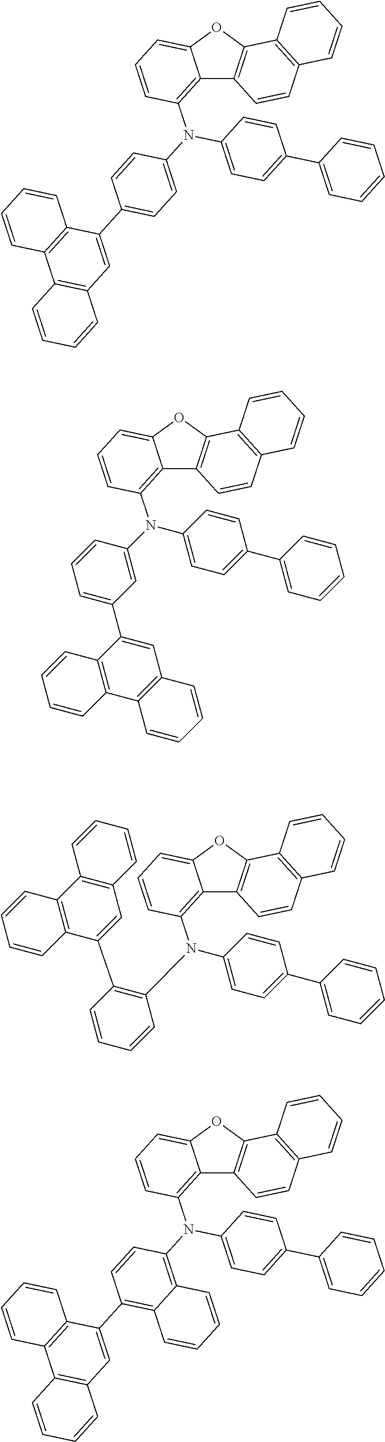 [SMILES: c1ccc(-c2ccc(N(c3ccc(-c4cc5ccccc5c5ccccc45)cc3)c3cccc4oc5c6ccccc6ccc5c34)cc2)cc1 ; c1ccc(-c2ccc(N(c3cccc(-c4cc5ccccc5c5ccccc45)c3)c3cccc4oc5c6ccccc6ccc5c34)cc2)cc1 ; c1ccc(-c2ccc(N(c3ccccc3-c3cc4ccccc4c4ccccc34)c3cccc4oc5c6ccccc6ccc5c34)cc2)cc1 ; c1ccc(-c2ccc(N(c3ccc(-c4cc5ccccc5c5ccccc45)c4ccccc34)c3cccc4oc5c6ccccc6ccc5c34)cc2)cc1]

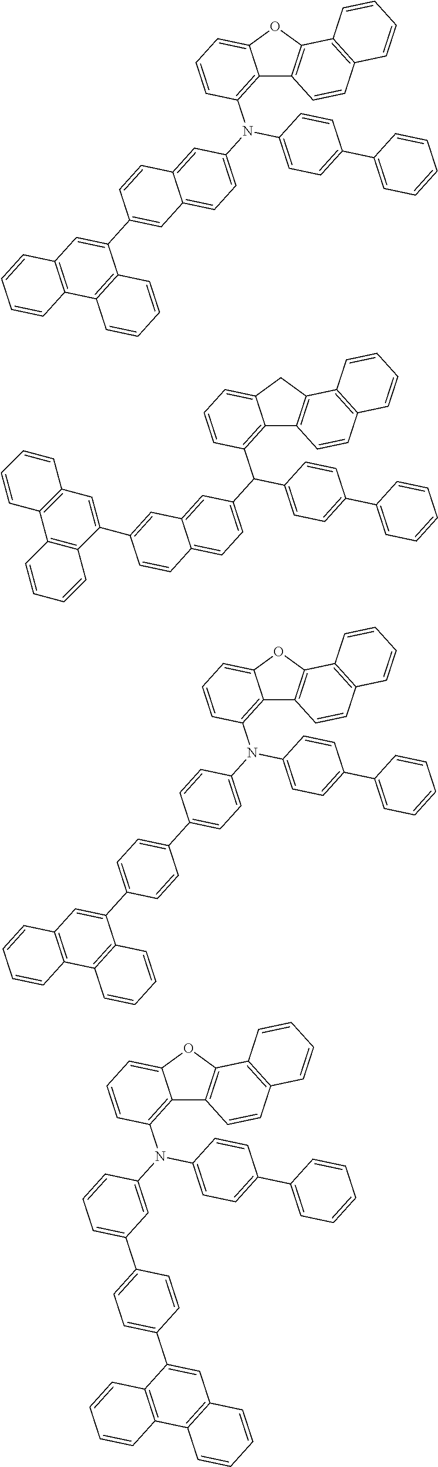

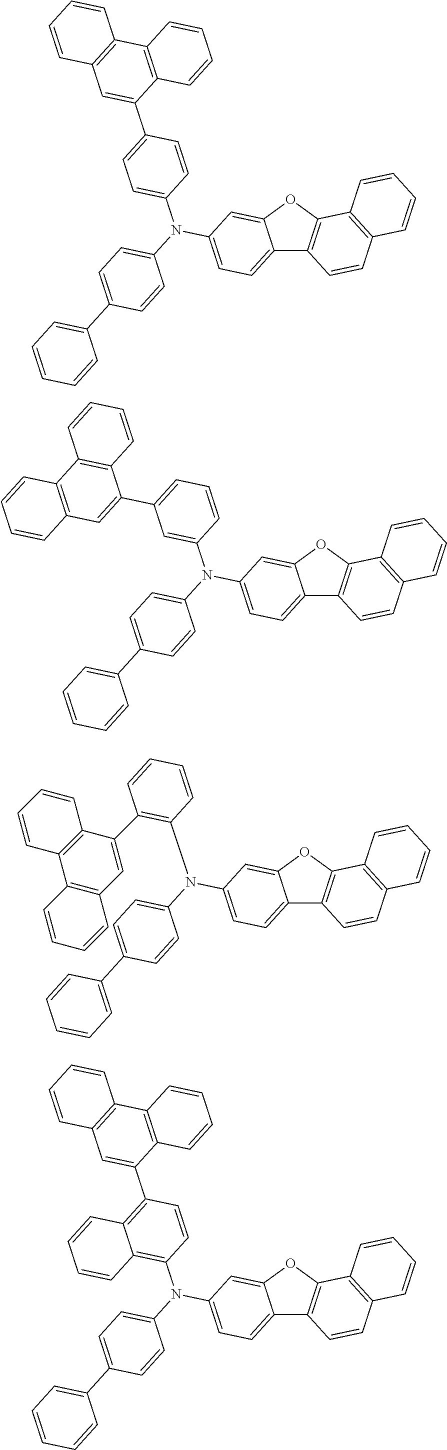

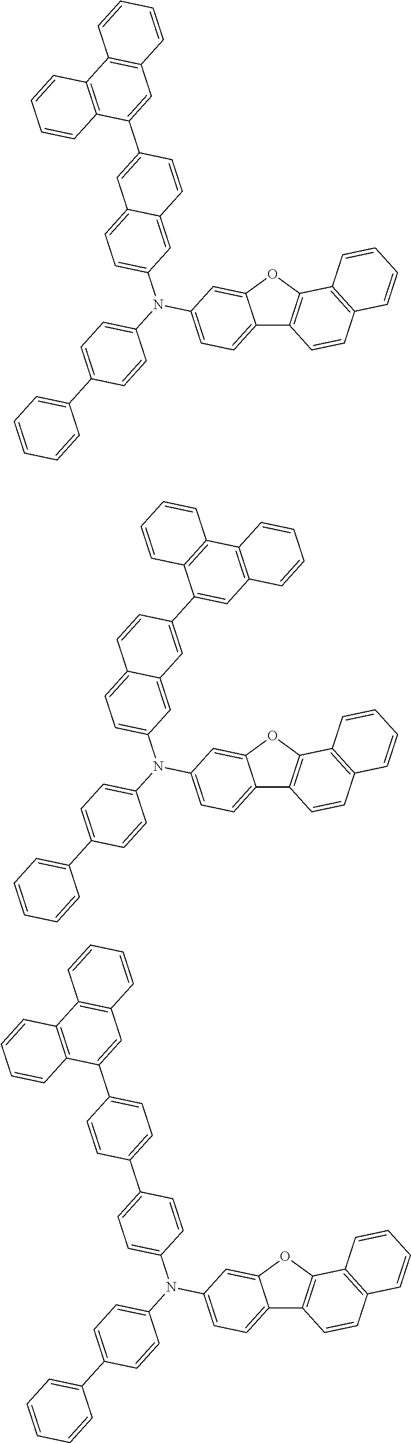

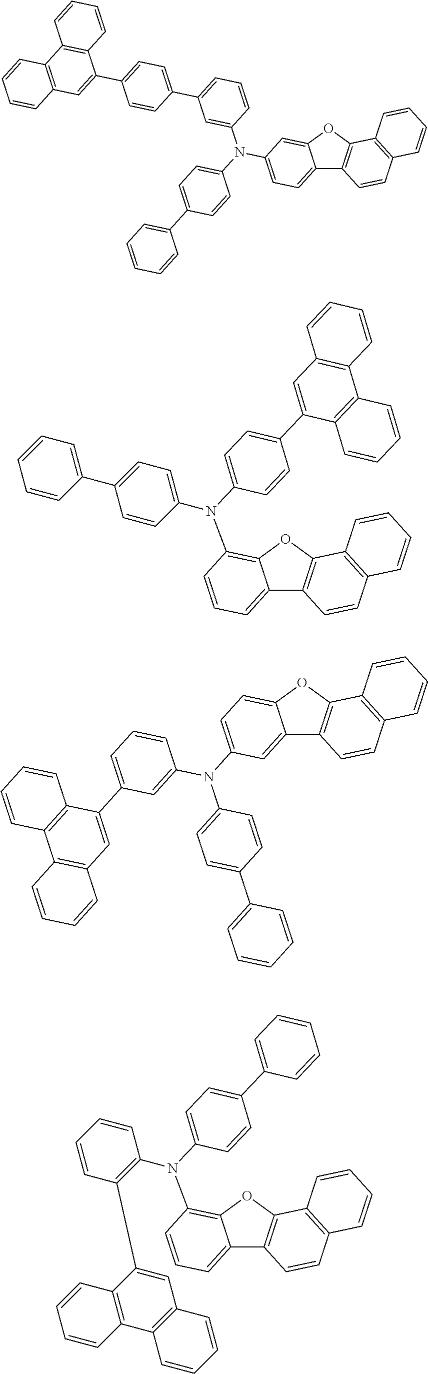 [SMILES: c1ccc(-c2ccc(N(c3cccc(-c4ccc(-c5cc6ccccc6c6ccccc56)cc4)c3)c3ccc4c(c3)oc3c5ccccc5ccc43)cc2)cc1 ; c1ccc(-c2ccc(N(c3ccc(-c4cc5ccccc5c5ccccc45)cc3)c3cccc4c3oc3c5ccccc5ccc43)cc2)cc1 ; c1ccc(-c2ccc(N(c3cccc(-c4cc5ccccc5c5ccccc45)c3)c3ccc4oc5c6ccccc6ccc5c4c3)cc2)cc1 ; c1ccc(-c2ccc(N(c3ccccc3-c3cc4ccccc4c4ccccc34)c3cccc4c3oc3c5ccccc5ccc43)cc2)cc1]

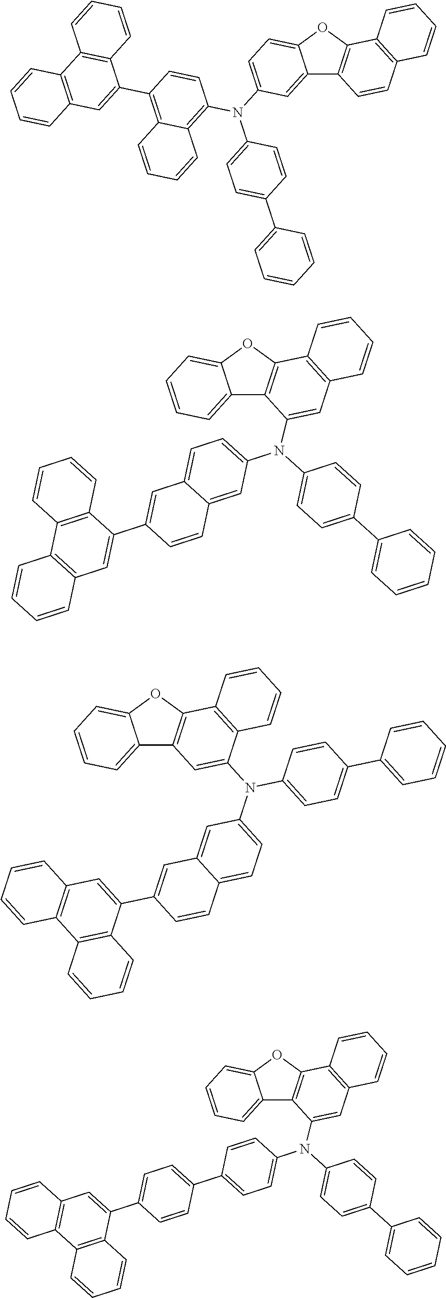 [SMILES: c1ccc(-c2ccc(N(c3ccc4oc5c6ccccc6ccc5c4c3)c3ccc(-c4cc5ccccc5c5ccccc45)c4ccccc34)cc2)cc1 ; c1ccc(-c2ccc(N(c3ccc4cc(-c5cc6ccccc6c6ccccc56)ccc4c3)c3cc4ccccc4c4oc5ccccc5c34)cc2)cc1 ; c1ccc(-c2ccc(N(c3ccc4ccc(-c5cc6ccccc6c6ccccc56)cc4c3)c3cc4c5ccccc5oc4c4ccccc34)cc2)cc1 ; c1ccc(-c2ccc(N(c3ccc(-c4ccc(-c5cc6ccccc6c6ccccc56)cc4)cc3)c3cc4ccccc4c4oc5ccccc5c34)cc2)cc1]

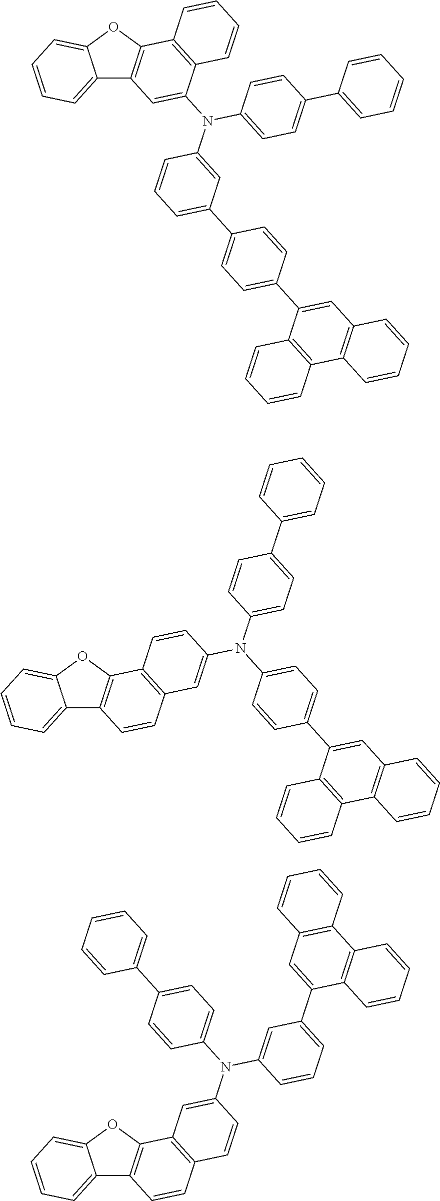

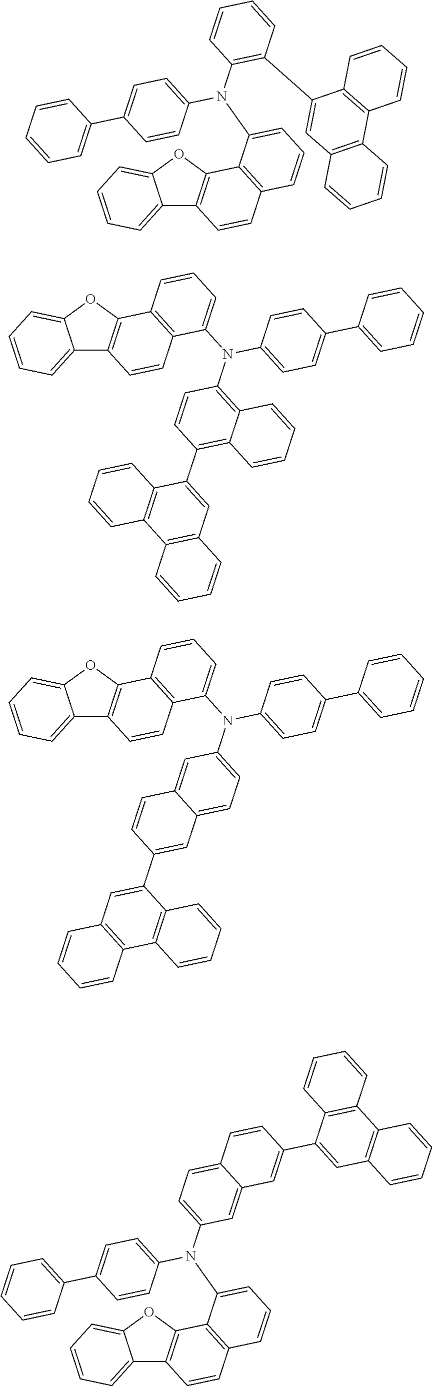

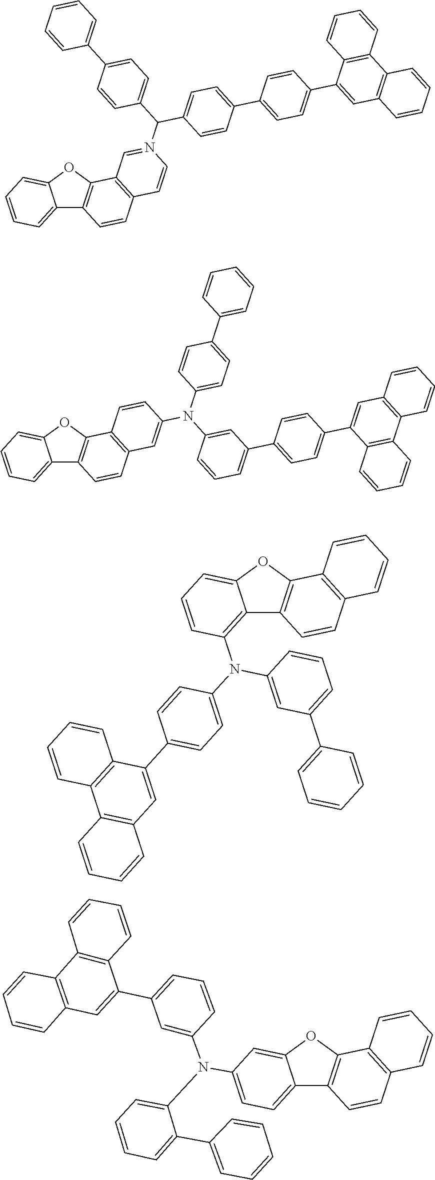

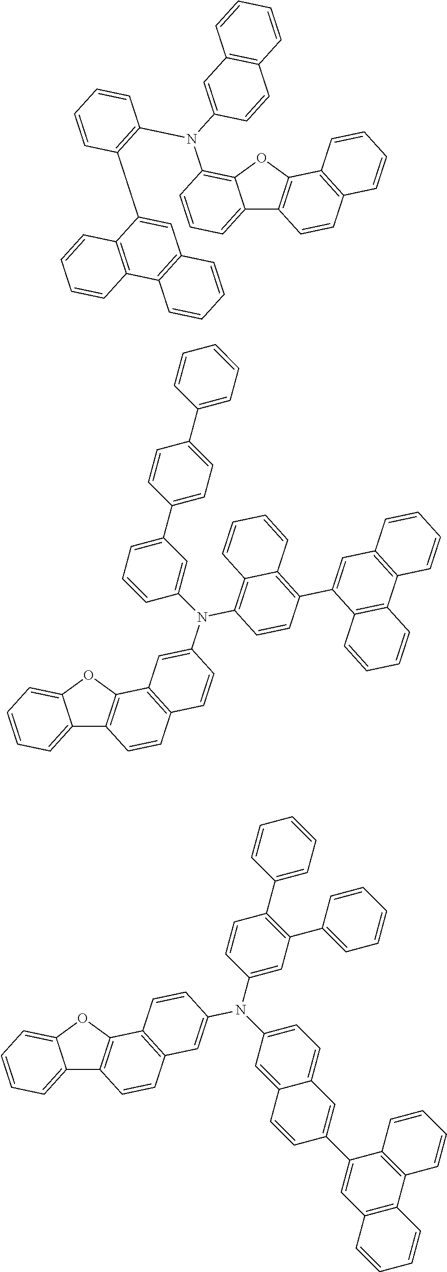

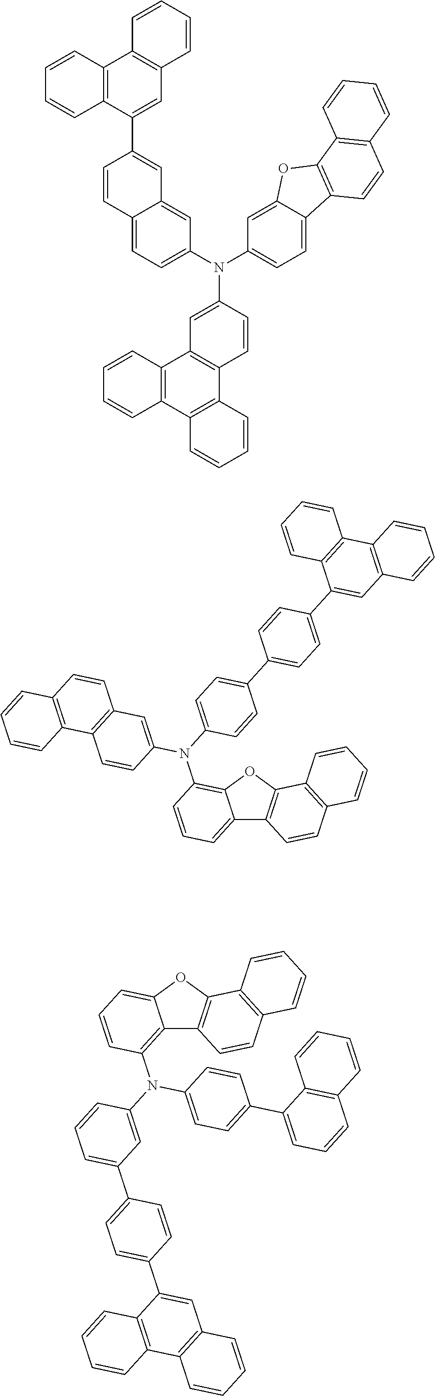 [SMILES: c1ccc2c(c1)cc(-c1ccc3ccc(N(c4ccc5c(c4)oc4c6ccccc6ccc54)c4ccc5c6ccccc6c6ccccc6c5c4)cc3c1)c1ccccc12 ; c1ccc2c(c1)ccc1cc(N(c3ccc(-c4ccc(-c5cc6ccccc6c6ccccc56)cc4)cc3)c3cccc4c3oc3c5ccccc5ccc43)ccc12 ; c1cc(-c2ccc(-c3cc4ccccc4c4ccccc34)cc2)cc(N(c2ccc(-c3cccc4ccccc34)cc2)c2cccc3oc4c5ccccc5ccc4c23)c1]

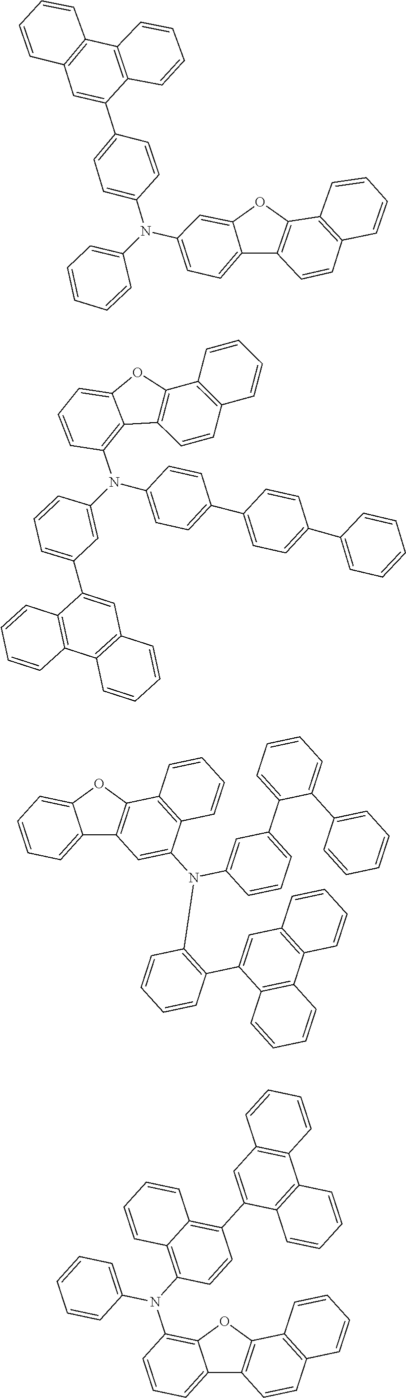

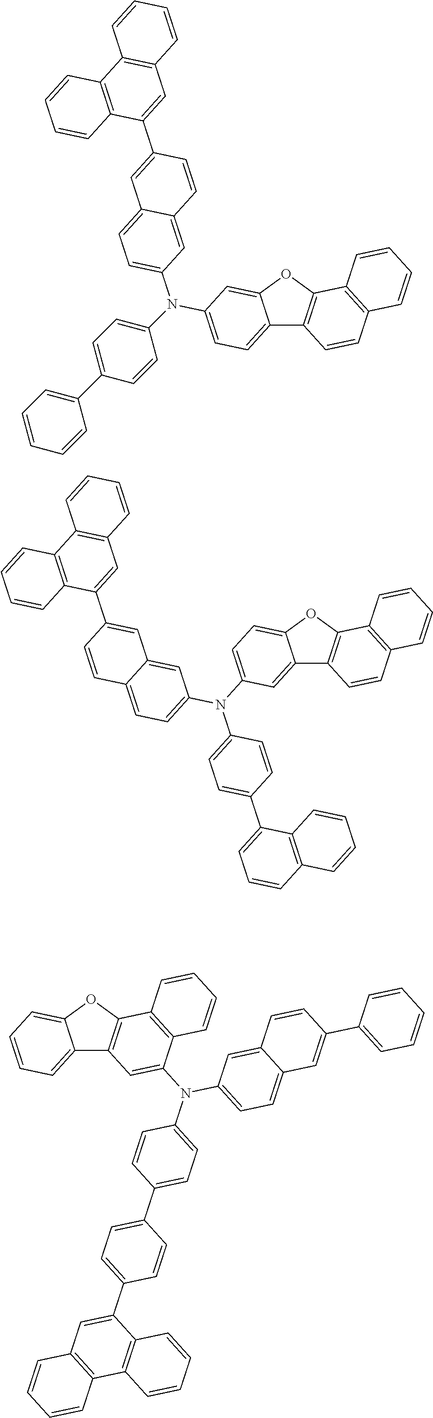

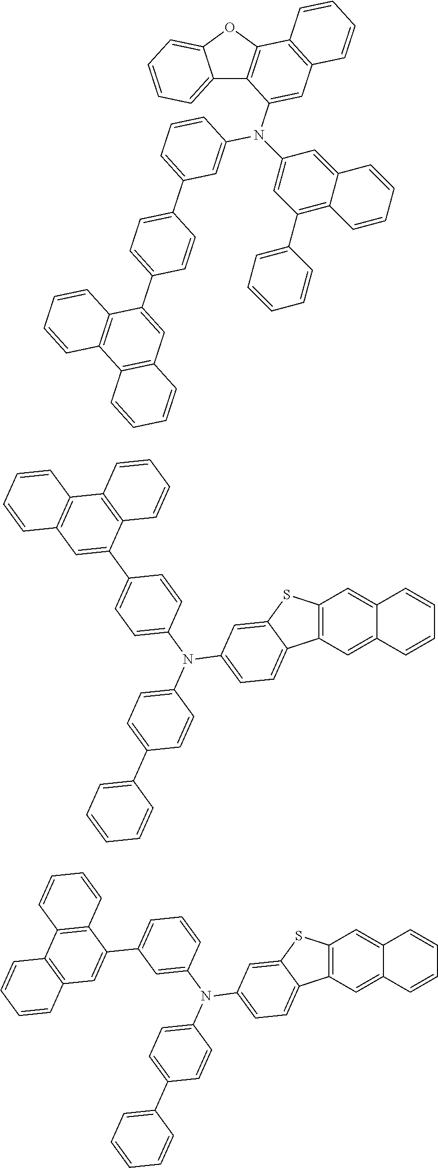

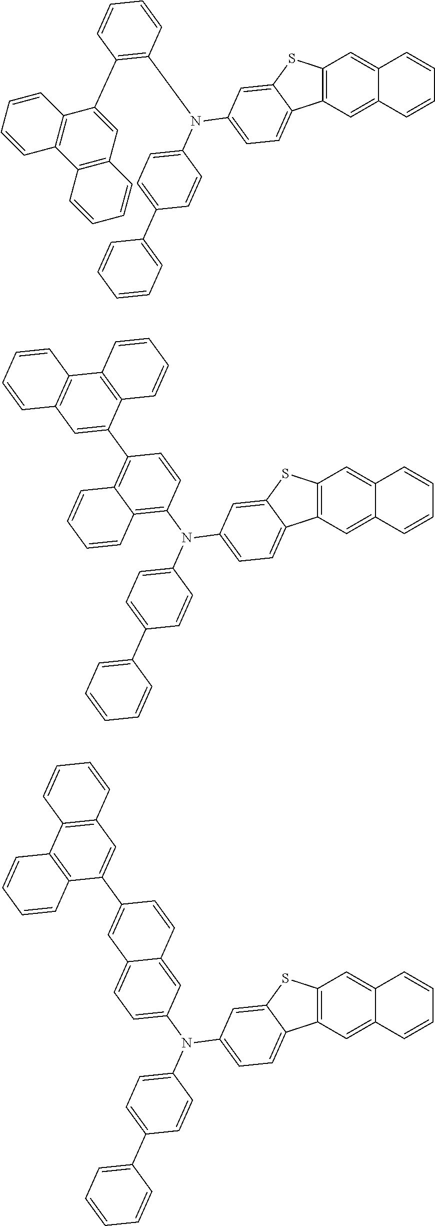

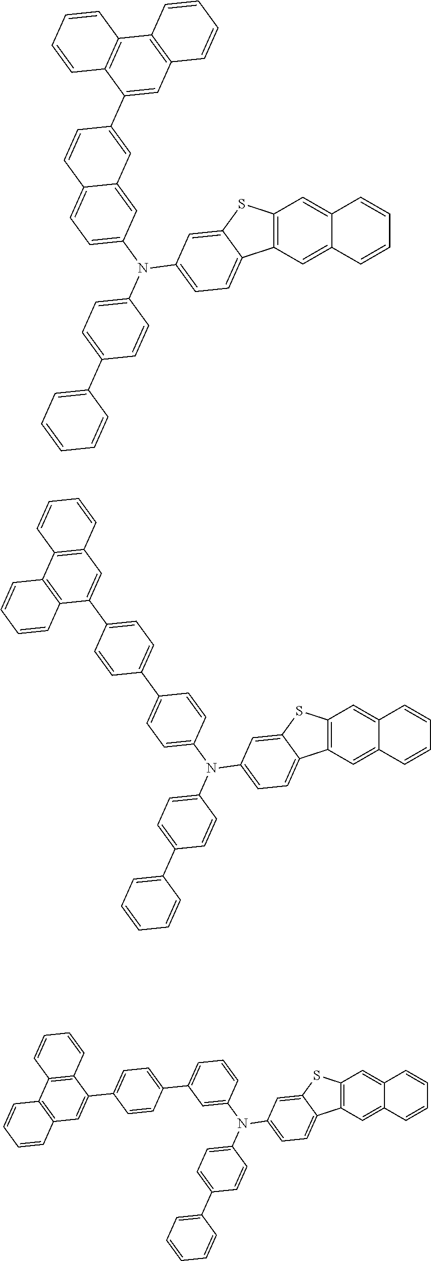 [SMILES: c1ccc(-c2ccc(N(c3ccc4ccc(-c5cc6ccccc6c6ccccc56)cc4c3)c3ccc4c(c3)sc3cc5ccccc5cc34)cc2)cc1 ; c1ccc(-c2ccc(N(c3ccc(-c4ccc(-c5cc6ccccc6c6ccccc56)cc4)cc3)c3ccc4c(c3)sc3cc5ccccc5cc34)cc2)cc1 ; c1ccc(-c2ccc(N(c3cccc(-c4ccc(-c5cc6ccccc6c6ccccc56)cc4)c3)c3ccc4c(c3)sc3cc5ccccc5cc34)cc2)cc1]

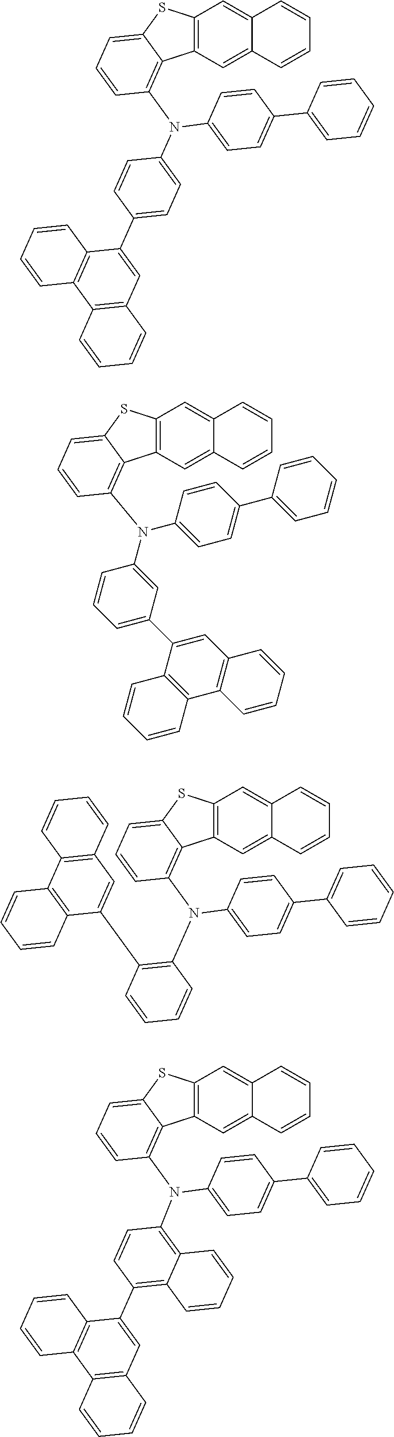

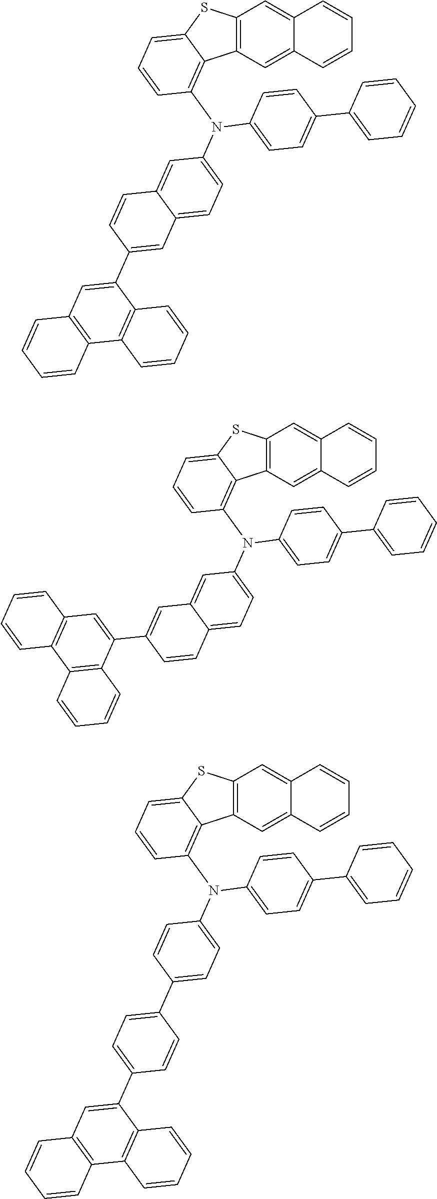 [SMILES: c1ccc(-c2ccc(N(c3ccc4cc(-c5cc6ccccc6c6ccccc56)ccc4c3)c3cccc4sc5cc6ccccc6cc5c34)cc2)cc1 ; c1ccc(-c2ccc(N(c3ccc4ccc(-c5cc6ccccc6c6ccccc56)cc4c3)c3cccc4sc5cc6ccccc6cc5c34)cc2)cc1 ; c1ccc(-c2ccc(N(c3ccc(-c4ccc(-c5cc6ccccc6c6ccccc56)cc4)cc3)c3cccc4sc5cc6ccccc6cc5c34)cc2)cc1]

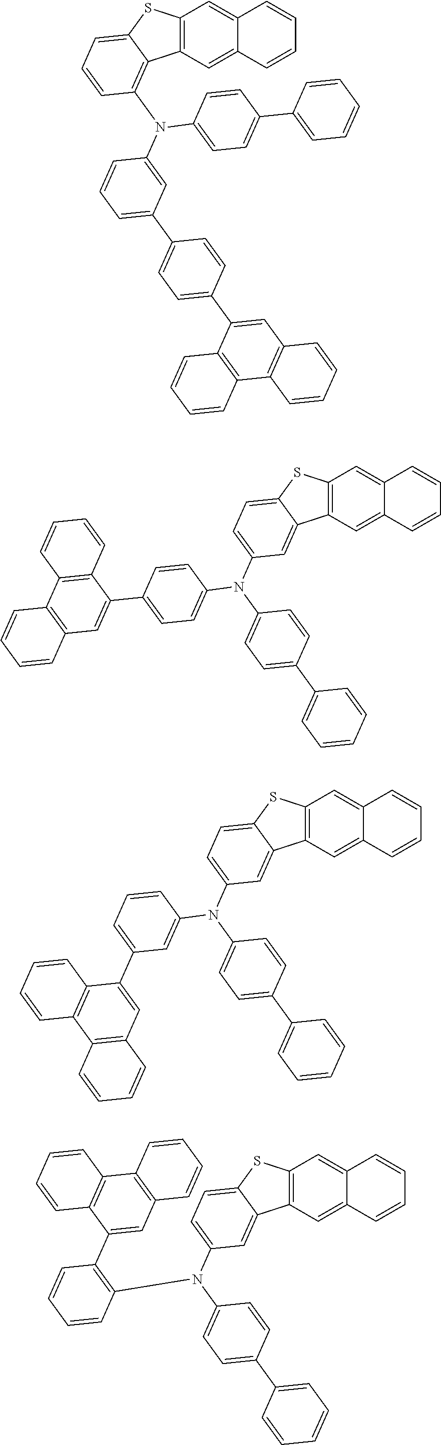

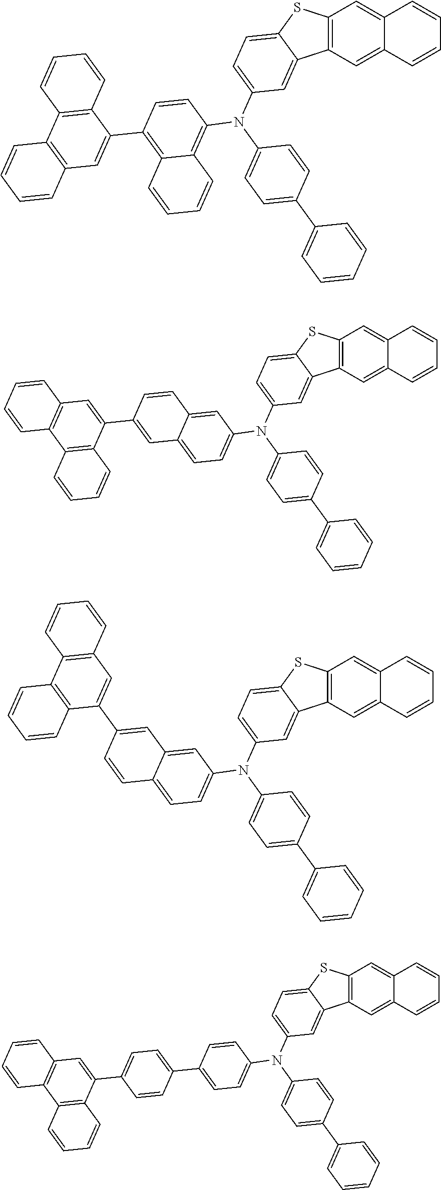 [SMILES: c1ccc(-c2ccc(N(c3ccc4sc5cc6ccccc6cc5c4c3)c3ccc(-c4cc5ccccc5c5ccccc45)c4ccccc34)cc2)cc1 ; c1ccc(-c2ccc(N(c3ccc4cc(-c5cc6ccccc6c6ccccc56)ccc4c3)c3ccc4sc5cc6ccccc6cc5c4c3)cc2)cc1 ; c1ccc(-c2ccc(N(c3ccc4ccc(-c5cc6ccccc6c6ccccc56)cc4c3)c3ccc4sc5cc6ccccc6cc5c4c3)cc2)cc1 ; c1ccc(-c2ccc(N(c3ccc(-c4ccc(-c5cc6ccccc6c6ccccc56)cc4)cc3)c3ccc4sc5cc6ccccc6cc5c4c3)cc2)cc1]

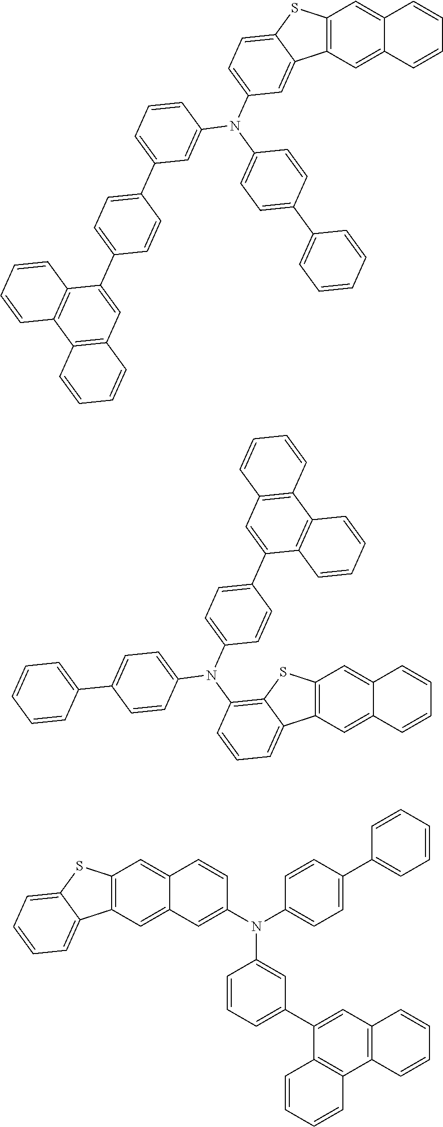 [SMILES: c1ccc(-c2ccc(N(c3cccc(-c4ccc(-c5cc6ccccc6c6ccccc56)cc4)c3)c3ccc4sc5cc6ccccc6cc5c4c3)cc2)cc1 ; c1ccc(-c2ccc(N(c3ccc(-c4cc5ccccc5c5ccccc45)cc3)c3cccc4c3sc3cc5ccccc5cc34)cc2)cc1 ; c1ccc(-c2ccc(N(c3cccc(-c4cc5ccccc5c5ccccc45)c3)c3ccc4cc5sc6ccccc6c5cc4c3)cc2)cc1]

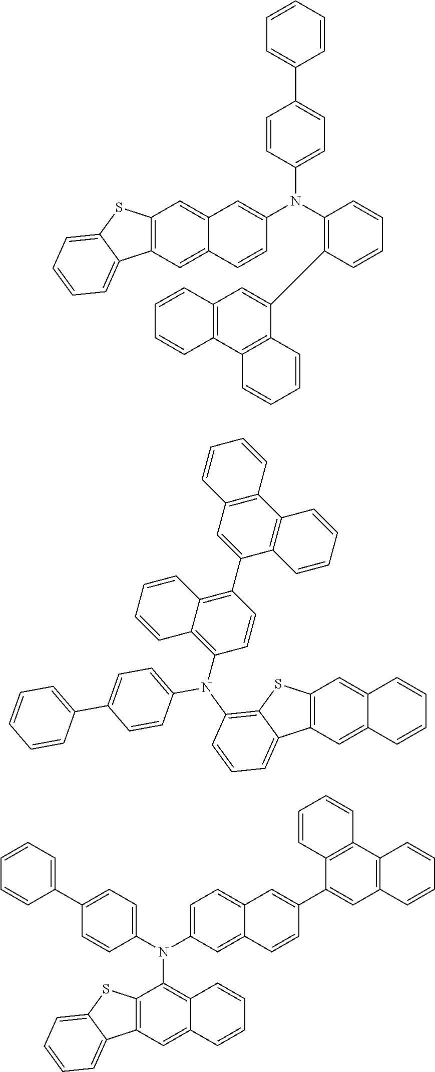

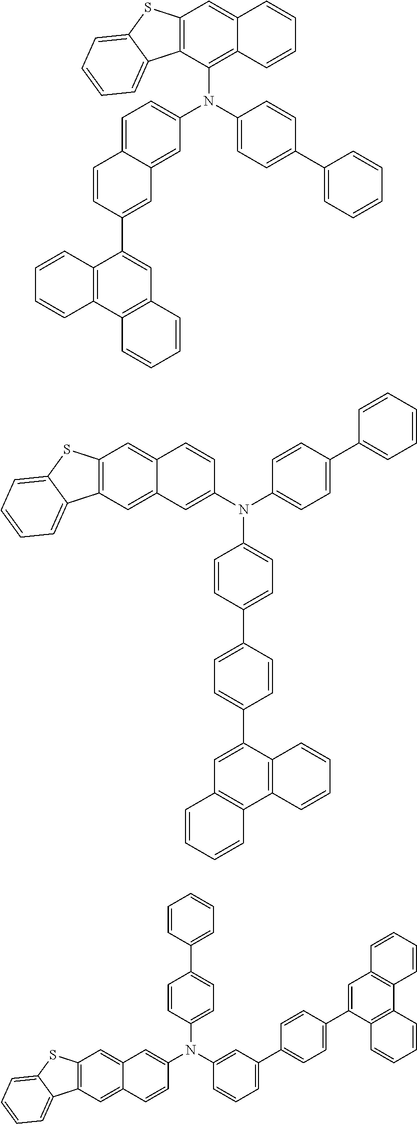

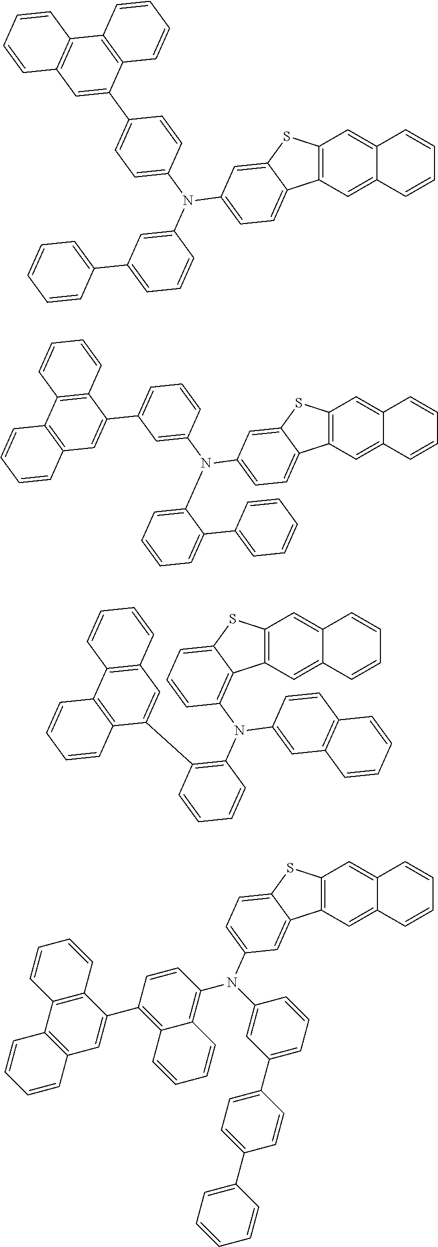

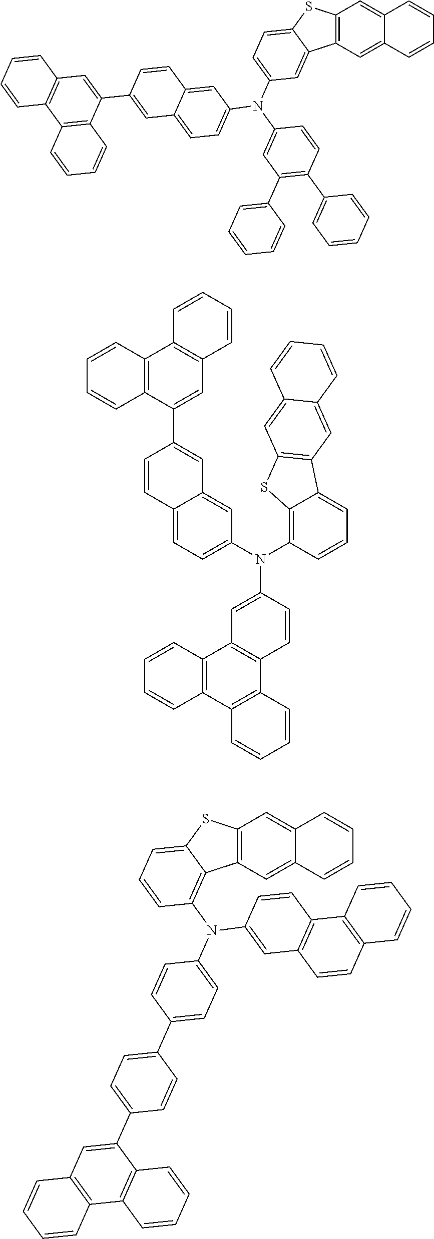 [SMILES: c1ccc(-c2ccc(N(c3ccc4cc(-c5cc6ccccc6c6ccccc56)ccc4c3)c3ccc4sc5cc6ccccc6cc5c4c3)cc2-c2ccccc2)cc1 ; c1ccc2cc3c(cc2c1)sc1c(N(c2ccc4ccc(-c5cc6ccccc6c6ccccc56)cc4c2)c2ccc4c5ccccc5c5ccccc5c4c2)cccc13 ; c1ccc2cc3c(cc2c1)sc1cccc(N(c2ccc(-c4ccc(-c5cc6ccccc6c6ccccc56)cc4)cc2)c2ccc4c(ccc5ccccc54)c2)c13]

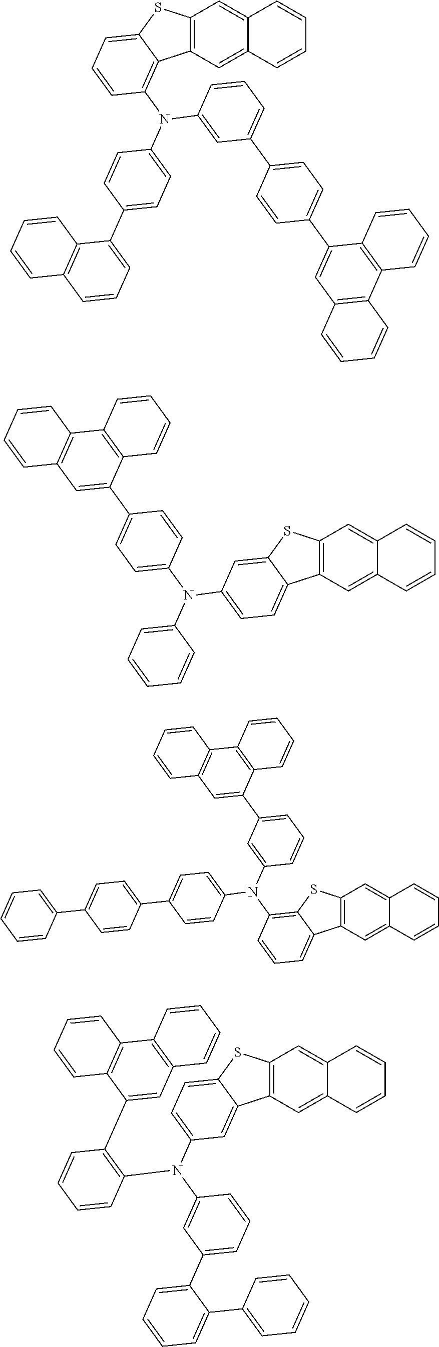

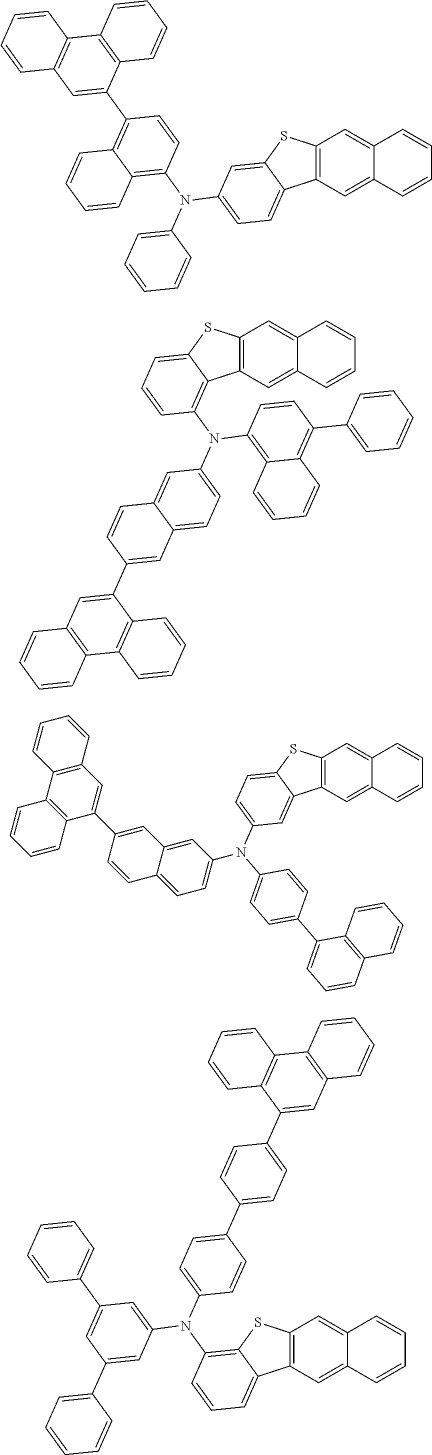

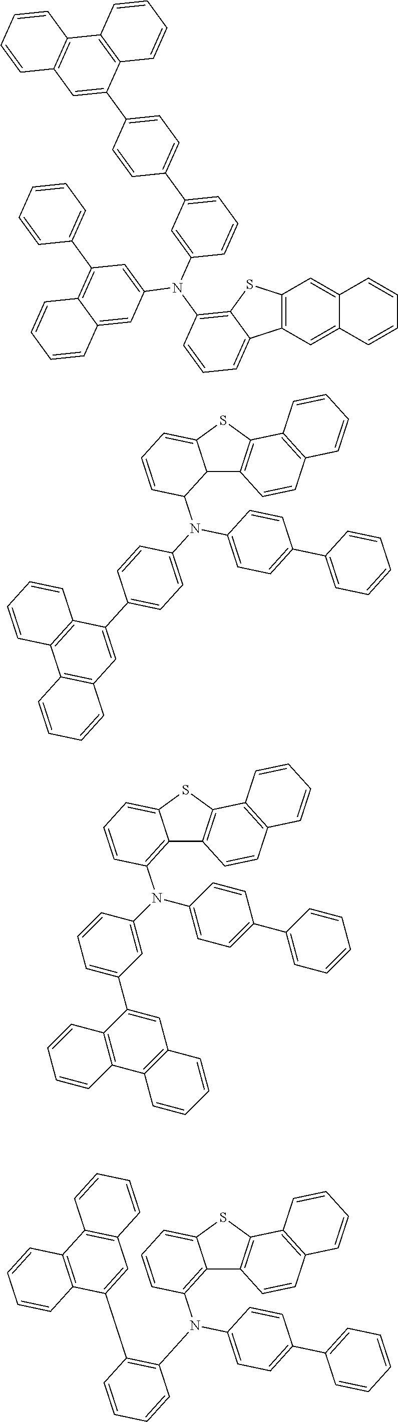 [SMILES: c1ccc(-c2cc(N(c3cccc(-c4ccc(-c5cc6ccccc6c6ccccc56)cc4)c3)c3cccc4c3sc3cc5ccccc5cc34)cc3ccccc23)cc1 ; C1=CC(N(c2ccc(-c3ccccc3)cc2)c2ccc(-c3cc4ccccc4c4ccccc34)cc2)C2C(=C1)Sc1c2ccc2ccccc12 ; c1ccc(-c2ccc(N(c3cccc(-c4cc5ccccc5c5ccccc45)c3)c3cccc4sc5c6ccccc6ccc5c34)cc2)cc1 ; c1ccc(-c2ccc(N(c3ccccc3-c3cc4ccccc4c4ccccc34)c3cccc4sc5c6ccccc6ccc5c34)cc2)cc1]

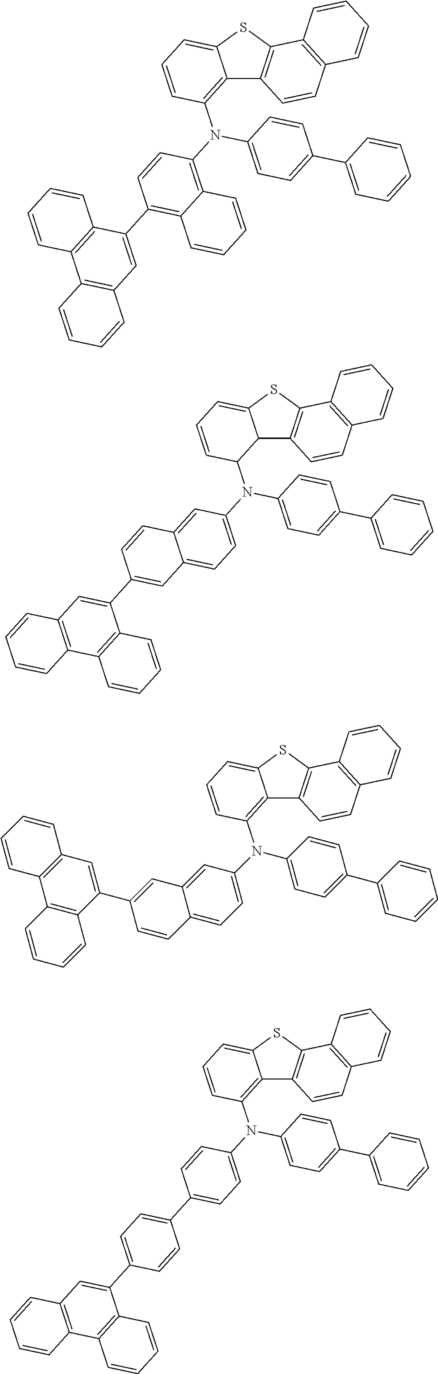 [SMILES: c1ccc(-c2ccc(N(c3ccc(-c4cc5ccccc5c5ccccc45)c4ccccc34)c3cccc4sc5c6ccccc6ccc5c34)cc2)cc1 ; C1=CC(N(c2ccc(-c3ccccc3)cc2)c2ccc3cc(-c4cc5ccccc5c5ccccc45)ccc3c2)C2C(=C1)Sc1c2ccc2ccccc12 ; c1ccc(-c2ccc(N(c3ccc4ccc(-c5cc6ccccc6c6ccccc56)cc4c3)c3cccc4sc5c6ccccc6ccc5c34)cc2)cc1 ; c1ccc(-c2ccc(N(c3ccc(-c4ccc(-c5cc6ccccc6c6ccccc56)cc4)cc3)c3cccc4sc5c6ccccc6ccc5c34)cc2)cc1]

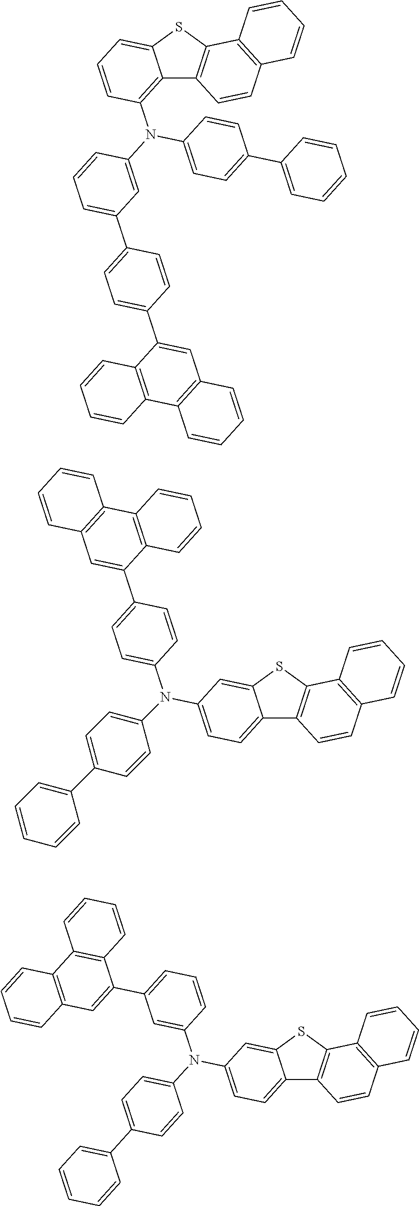 [SMILES: c1ccc(-c2ccc(N(c3cccc(-c4ccc(-c5cc6ccccc6c6ccccc56)cc4)c3)c3cccc4sc5c6ccccc6ccc5c34)cc2)cc1 ; c1ccc(-c2ccc(N(c3ccc(-c4cc5ccccc5c5ccccc45)cc3)c3ccc4c(c3)sc3c5ccccc5ccc43)cc2)cc1 ; c1ccc(-c2ccc(N(c3cccc(-c4cc5ccccc5c5ccccc45)c3)c3ccc4c(c3)sc3c5ccccc5ccc43)cc2)cc1]

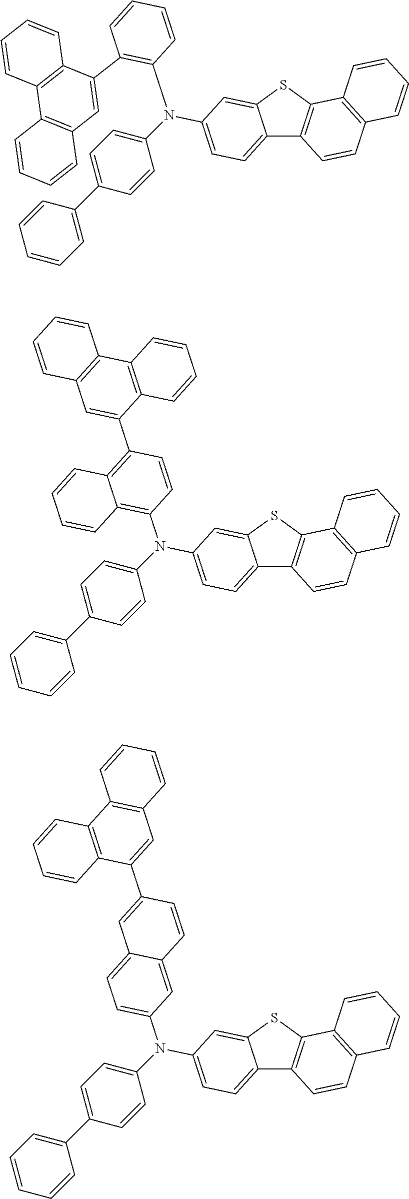 [SMILES: c1ccc(-c2ccc(N(c3ccc4c(c3)sc3c5ccccc5ccc43)c3ccccc3-c3cc4ccccc4c4ccccc34)cc2)cc1 ; c1ccc(-c2ccc(N(c3ccc4c(c3)sc3c5ccccc5ccc43)c3ccc(-c4cc5ccccc5c5ccccc45)c4ccccc34)cc2)cc1 ; c1ccc(-c2ccc(N(c3ccc4cc(-c5cc6ccccc6c6ccccc56)ccc4c3)c3ccc4c(c3)sc3c5ccccc5ccc43)cc2)cc1]

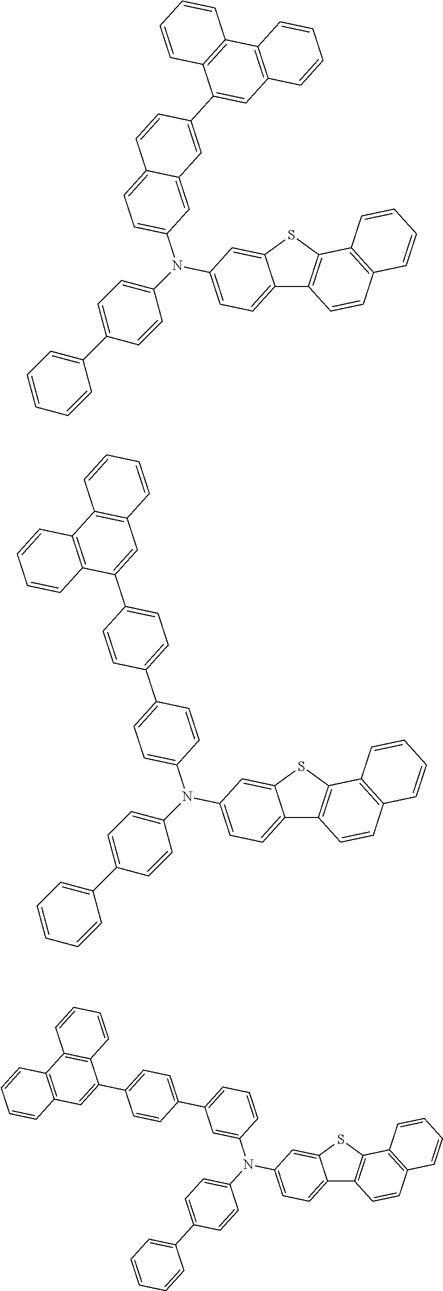 [SMILES: c1ccc(-c2ccc(N(c3ccc4ccc(-c5cc6ccccc6c6ccccc56)cc4c3)c3ccc4c(c3)sc3c5ccccc5ccc43)cc2)cc1 ; c1ccc(-c2ccc(N(c3ccc(-c4ccc(-c5cc6ccccc6c6ccccc56)cc4)cc3)c3ccc4c(c3)sc3c5ccccc5ccc43)cc2)cc1 ; c1ccc(-c2ccc(N(c3cccc(-c4ccc(-c5cc6ccccc6c6ccccc56)cc4)c3)c3ccc4c(c3)sc3c5ccccc5ccc43)cc2)cc1]

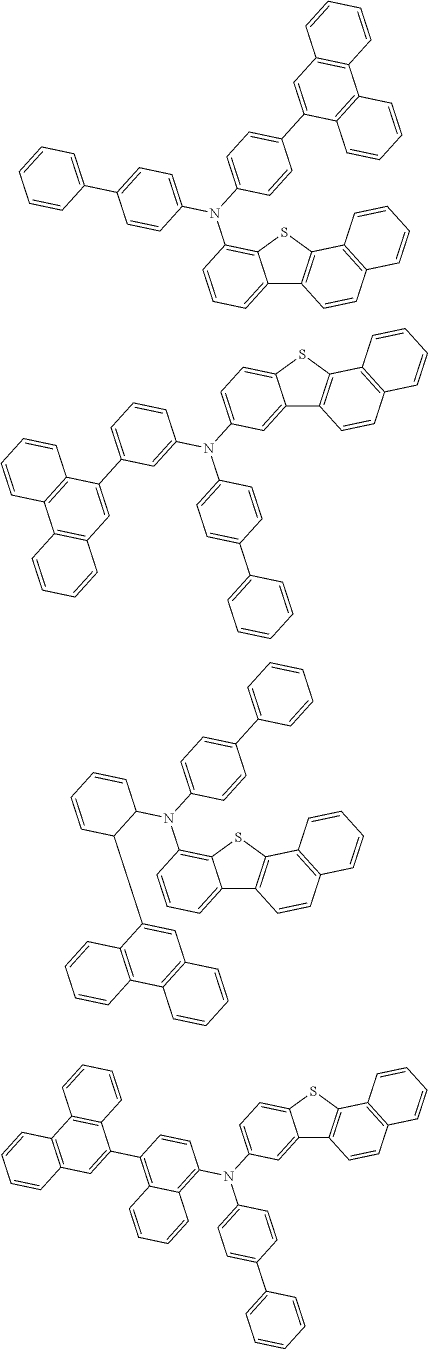

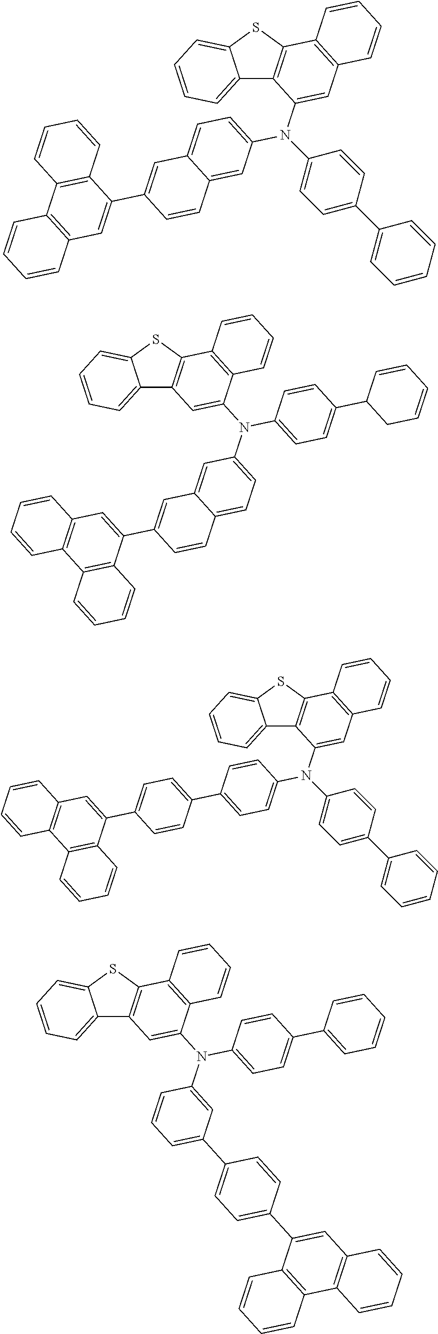 [SMILES: c1ccc(-c2ccc(N(c3ccc4cc(-c5cc6ccccc6c6ccccc56)ccc4c3)c3cc4ccccc4c4sc5ccccc5c34)cc2)cc1 ; C1=CCC(c2ccc(N(c3ccc4ccc(-c5cc6ccccc6c6ccccc56)cc4c3)c3cc4c5ccccc5sc4c4ccccc34)cc2)C=C1 ; c1ccc(-c2ccc(N(c3ccc(-c4ccc(-c5cc6ccccc6c6ccccc56)cc4)cc3)c3cc4ccccc4c4sc5ccccc5c34)cc2)cc1 ; c1ccc(-c2ccc(N(c3cccc(-c4ccc(-c5cc6ccccc6c6ccccc56)cc4)c3)c3cc4c5ccccc5sc4c4ccccc34)cc2)cc1]

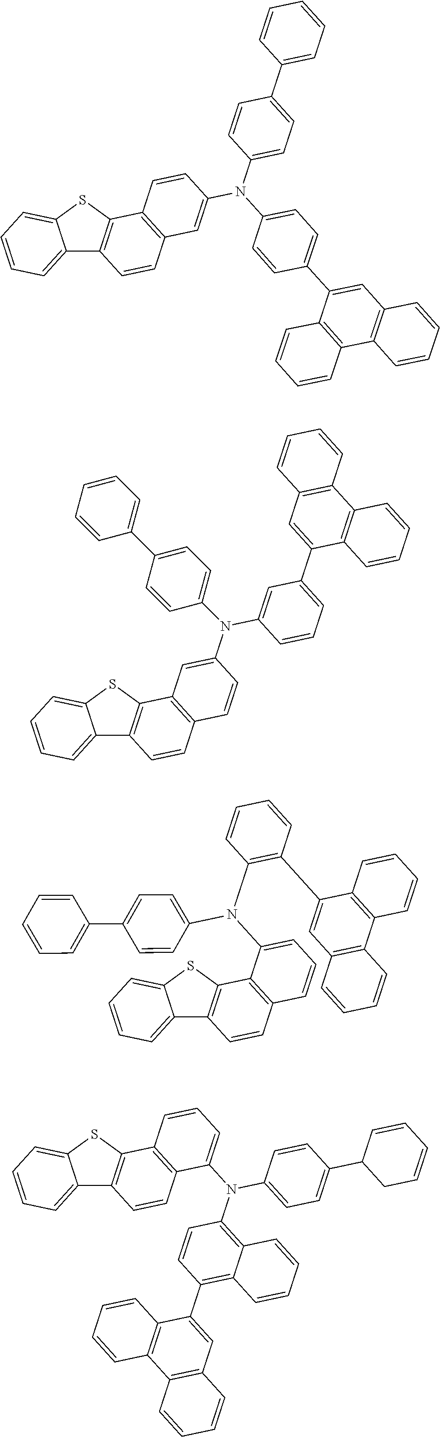 [SMILES: c1ccc(-c2ccc(N(c3ccc(-c4cc5ccccc5c5ccccc45)cc3)c3ccc4c(ccc5c6ccccc6sc45)c3)cc2)cc1 ; c1ccc(-c2ccc(N(c3cccc(-c4cc5ccccc5c5ccccc45)c3)c3ccc4ccc5c6ccccc6sc5c4c3)cc2)cc1 ; c1ccc(-c2ccc(N(c3ccccc3-c3cc4ccccc4c4ccccc34)c3cccc4ccc5c6ccccc6sc5c34)cc2)cc1 ; C1=CCC(c2ccc(N(c3ccc(-c4cc5ccccc5c5ccccc45)c4ccccc34)c3cccc4c3ccc3c5ccccc5sc43)cc2)C=C1]

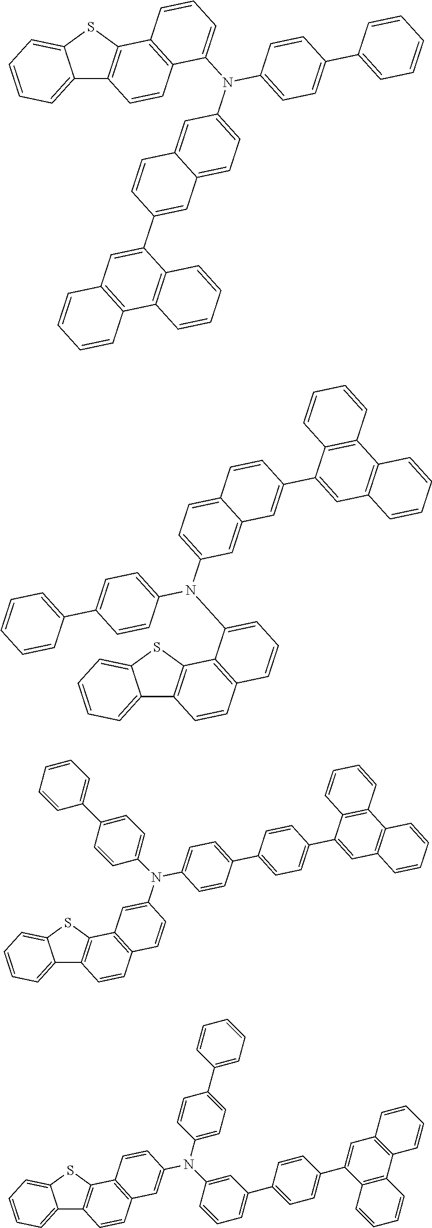 [SMILES: c1ccc(-c2ccc(N(c3ccc4cc(-c5cc6ccccc6c6ccccc56)ccc4c3)c3cccc4c3ccc3c5ccccc5sc43)cc2)cc1 ; c1ccc(-c2ccc(N(c3ccc4ccc(-c5cc6ccccc6c6ccccc56)cc4c3)c3cccc4ccc5c6ccccc6sc5c34)cc2)cc1 ; c1ccc(-c2ccc(N(c3ccc(-c4ccc(-c5cc6ccccc6c6ccccc56)cc4)cc3)c3ccc4ccc5c6ccccc6sc5c4c3)cc2)cc1 ; c1ccc(-c2ccc(N(c3cccc(-c4ccc(-c5cc6ccccc6c6ccccc56)cc4)c3)c3ccc4c(ccc5c6ccccc6sc45)c3)cc2)cc1]

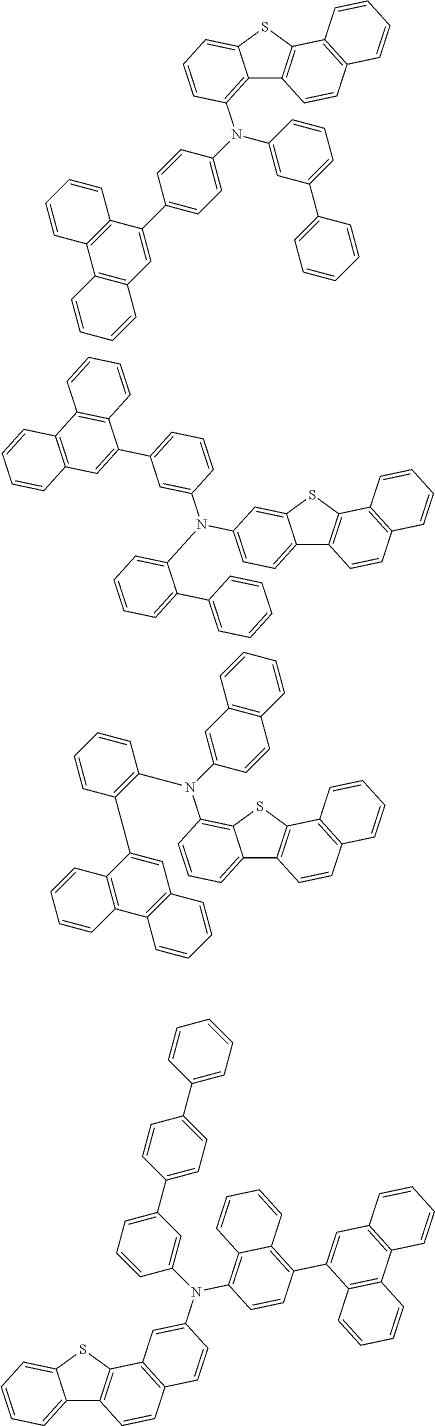

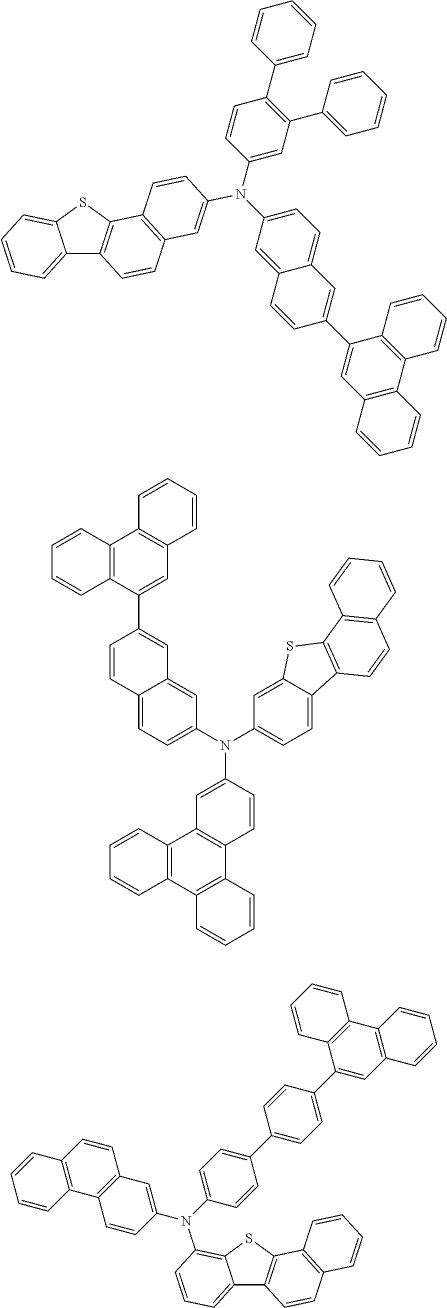 [SMILES: c1ccc(-c2ccc(N(c3ccc4cc(-c5cc6ccccc6c6ccccc56)ccc4c3)c3ccc4c(ccc5c6ccccc6sc45)c3)cc2-c2ccccc2)cc1 ; c1ccc2c(c1)cc(-c1ccc3ccc(N(c4ccc5c(c4)sc4c6ccccc6ccc54)c4ccc5c6ccccc6c6ccccc6c5c4)cc3c1)c1ccccc12 ; c1ccc2c(c1)ccc1cc(N(c3ccc(-c4ccc(-c5cc6ccccc6c6ccccc56)cc4)cc3)c3cccc4c3sc3c5ccccc5ccc43)ccc12]

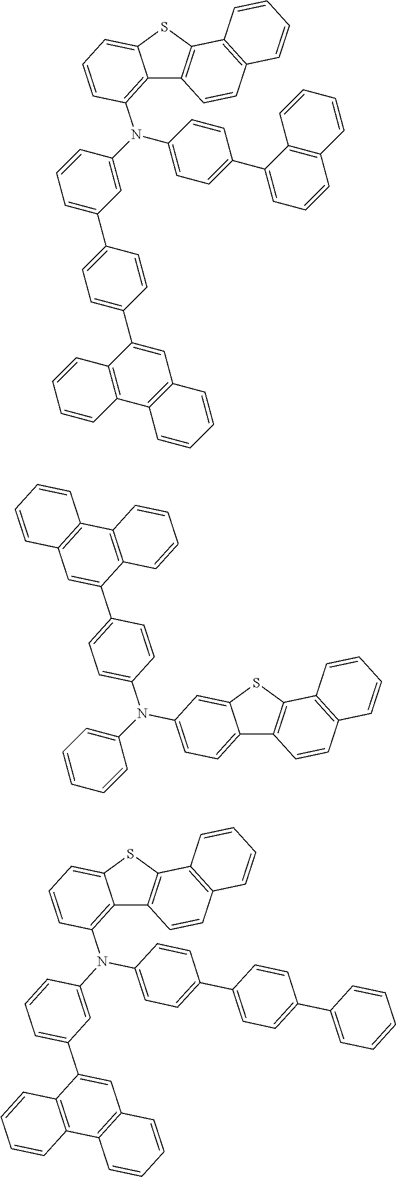 [SMILES: c1cc(-c2ccc(-c3cc4ccccc4c4ccccc34)cc2)cc(N(c2ccc(-c3cccc4ccccc34)cc2)c2cccc3sc4c5ccccc5ccc4c23)c1 ; c1ccc(N(c2ccc(-c3cc4ccccc4c4ccccc34)cc2)c2ccc3c(c2)sc2c4ccccc4ccc32)cc1 ; c1ccc(-c2ccc(-c3ccc(N(c4cccc(-c5cc6ccccc6c6ccccc56)c4)c4cccc5sc6c7ccccc7ccc6c45)cc3)cc2)cc1]

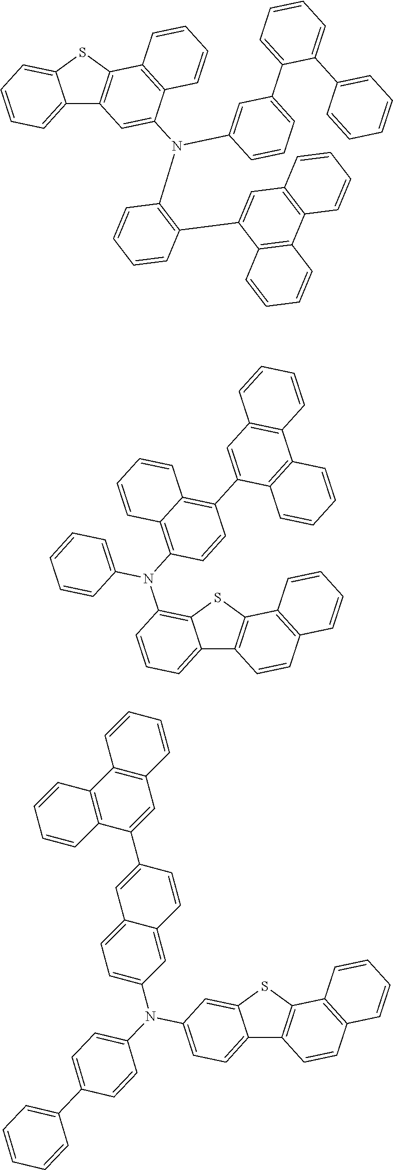 [SMILES: c1ccc(-c2ccccc2-c2cccc(N(c3ccccc3-c3cc4ccccc4c4ccccc34)c3cc4c5ccccc5sc4c4ccccc34)c2)cc1 ; c1ccc(N(c2ccc(-c3cc4ccccc4c4ccccc34)c3ccccc23)c2cccc3c2sc2c4ccccc4ccc32)cc1 ; c1ccc(-c2ccc(N(c3ccc4cc(-c5cc6ccccc6c6ccccc56)ccc4c3)c3ccc4c(c3)sc3c5ccccc5ccc43)cc2)cc1]

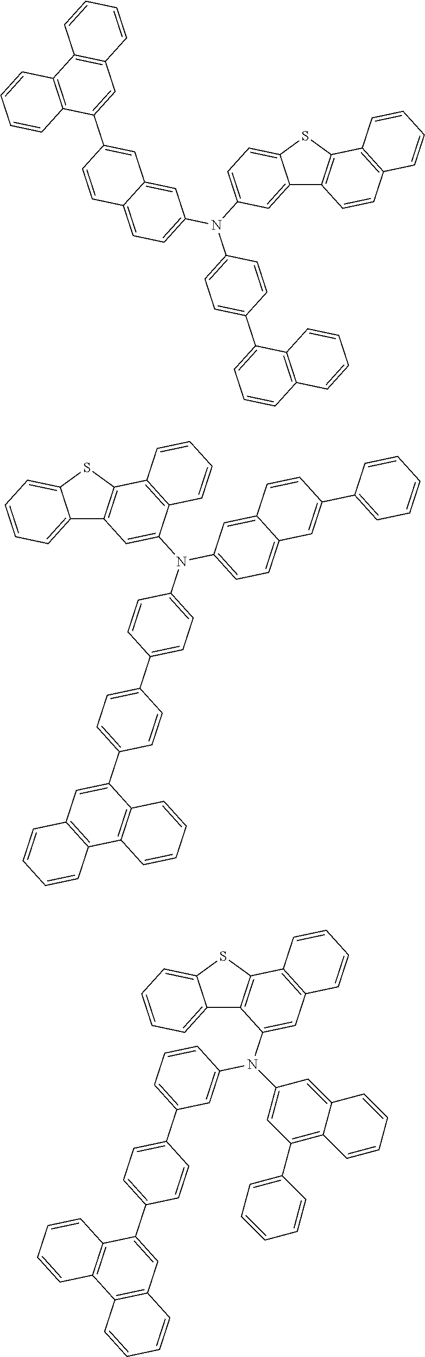 [SMILES: c1ccc2c(-c3ccc(N(c4ccc5ccc(-c6cc7ccccc7c7ccccc67)cc5c4)c4ccc5sc6c7ccccc7ccc6c5c4)cc3)cccc2c1 ; c1ccc(-c2ccc3cc(N(c4ccc(-c5ccc(-c6cc7ccccc7c7ccccc67)cc5)cc4)c4cc5c6ccccc6sc5c5ccccc45)ccc3c2)cc1 ; c1ccc(-c2cc(N(c3cccc(-c4ccc(-c5cc6ccccc6c6ccccc56)cc4)c3)c3cc4ccccc4c4sc5ccccc5c34)cc3ccccc23)cc1]

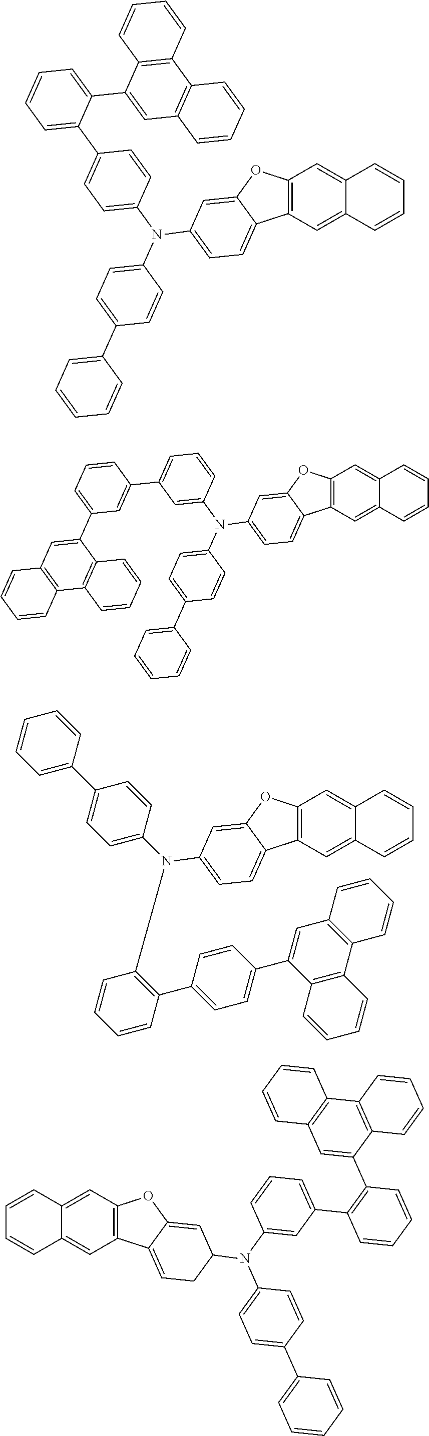

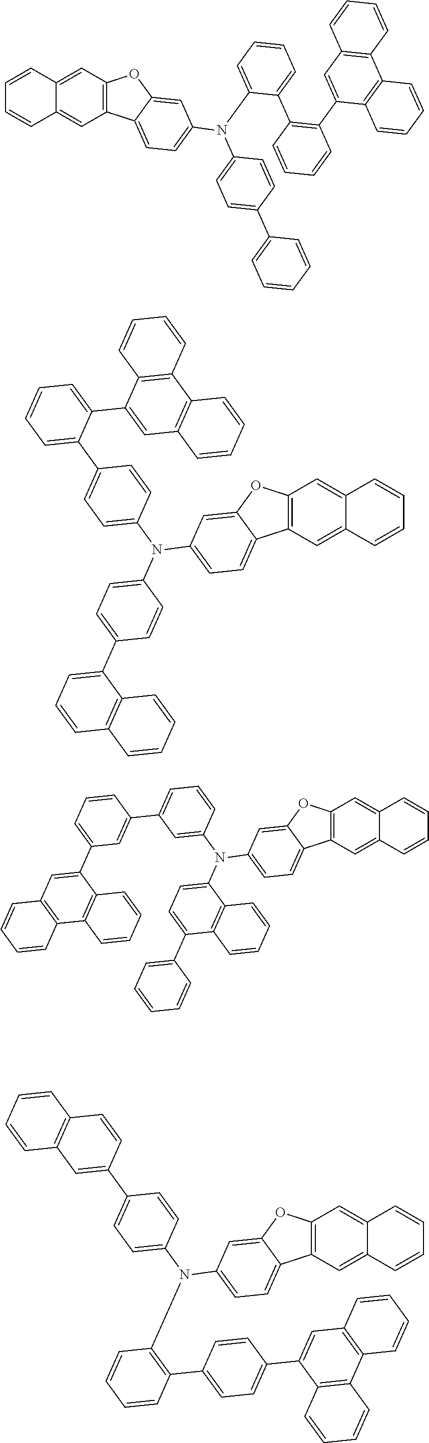

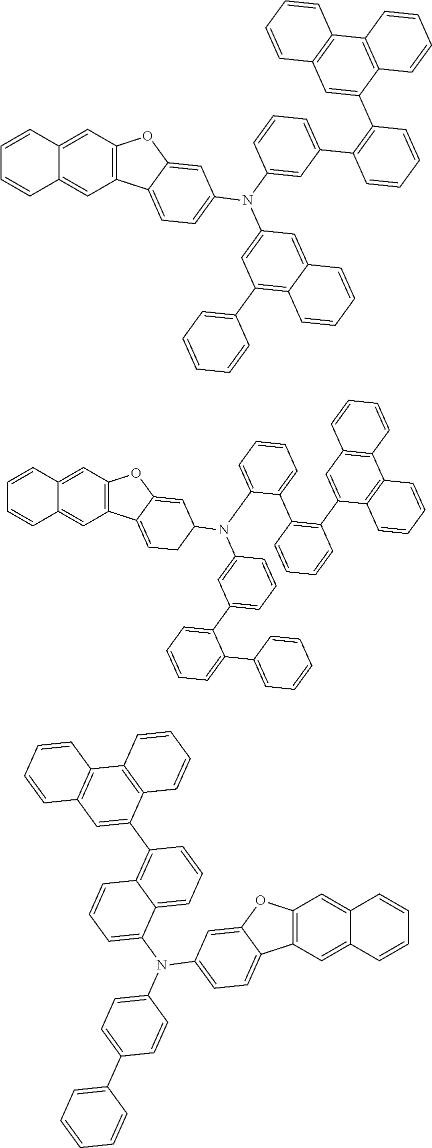 [SMILES: c1ccc(-c2cc(N(c3cccc(-c4ccccc4-c4cc5ccccc5c5ccccc45)c3)c3ccc4c(c3)oc3cc5ccccc5cc34)cc3ccccc23)cc1 ; C1=c2oc3cc4ccccc4cc3c2=CCC1N(c1cccc(-c2ccccc2-c2ccccc2)c1)c1ccccc1-c1ccccc1-c1cc2ccccc2c2ccccc12 ; c1ccc(-c2ccc(N(c3ccc4c(c3)oc3cc5ccccc5cc34)c3cccc4c(-c5cc6ccccc6c6ccccc56)cccc34)cc2)cc1]

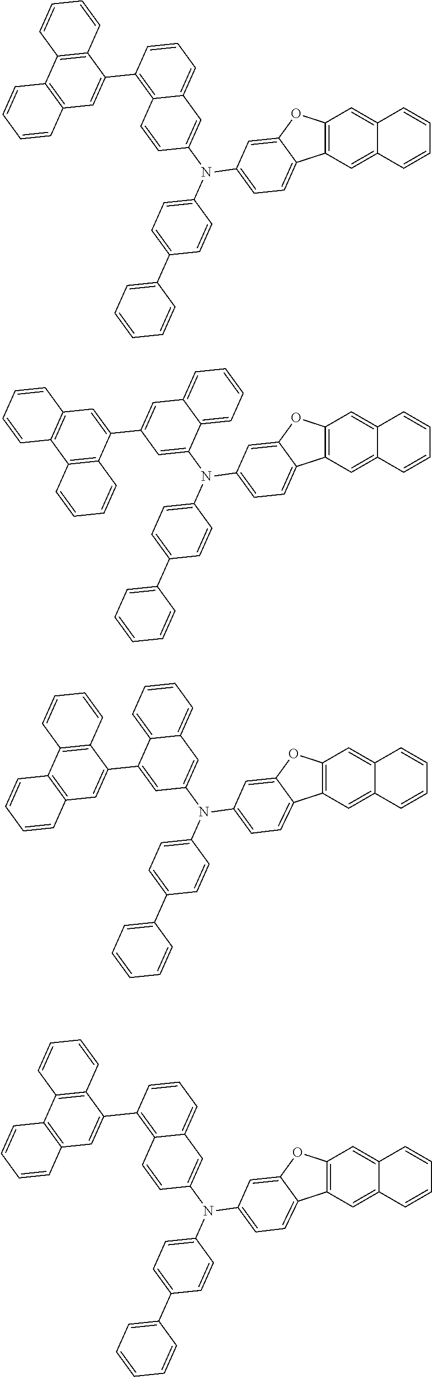 [SMILES: c1ccc(-c2ccc(N(c3ccc4c(-c5cc6ccccc6c6ccccc56)cccc4c3)c3ccc4c(c3)oc3cc5ccccc5cc34)cc2)cc1 ; c1ccc(-c2ccc(N(c3ccc4c(c3)oc3cc5ccccc5cc34)c3cc(-c4cc5ccccc5c5ccccc45)cc4ccccc34)cc2)cc1 ; c1ccc(-c2ccc(N(c3cc(-c4cc5ccccc5c5ccccc45)c4ccccc4c3)c3ccc4c(c3)oc3cc5ccccc5cc34)cc2)cc1 ; c1ccc(-c2ccc(N(c3ccc4c(-c5cc6ccccc6c6ccccc56)cccc4c3)c3ccc4c(c3)oc3cc5ccccc5cc34)cc2)cc1]

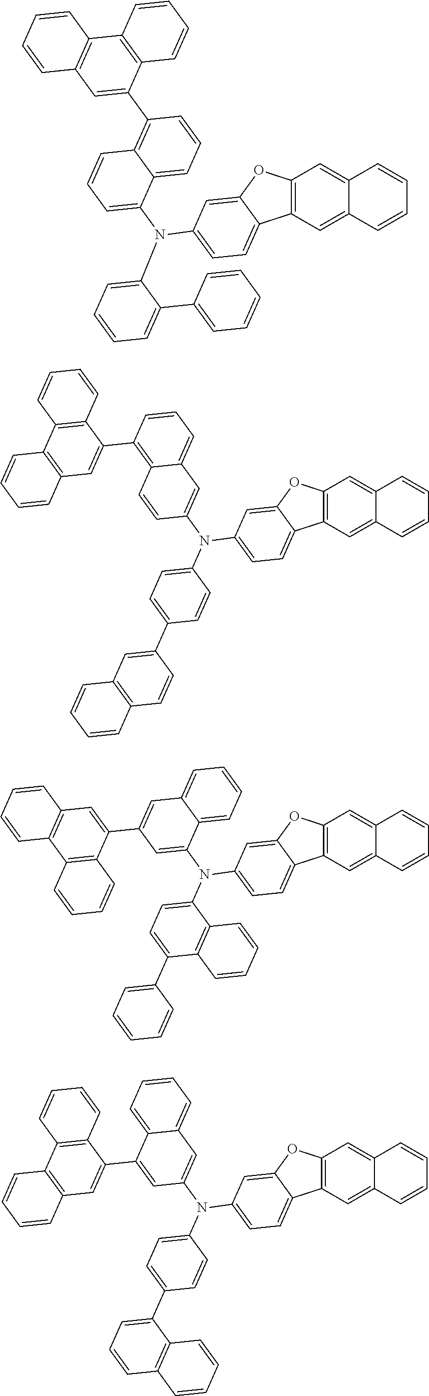 [SMILES: c1ccc(-c2ccccc2N(c2ccc3c(c2)oc2cc4ccccc4cc23)c2cccc3c(-c4cc5ccccc5c5ccccc45)cccc23)cc1 ; c1ccc2cc(-c3ccc(N(c4ccc5c(-c6cc7ccccc7c7ccccc67)cccc5c4)c4ccc5c(c4)oc4cc6ccccc6cc45)cc3)ccc2c1 ; c1ccc(-c2ccc(N(c3ccc4c(c3)oc3cc5ccccc5cc34)c3cc(-c4cc5ccccc5c5ccccc45)cc4ccccc34)c3ccccc23)cc1 ; c1ccc2cc3c(cc2c1)oc1cc(N(c2ccc(-c4cccc5ccccc45)cc2)c2cc(-c4cc5ccccc5c5ccccc45)c4ccccc4c2)ccc13]

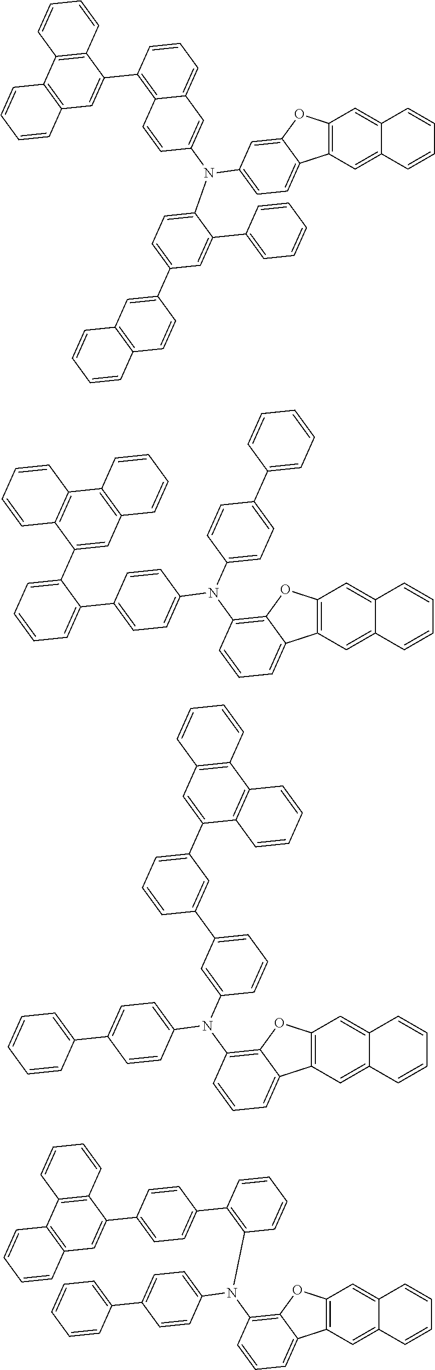

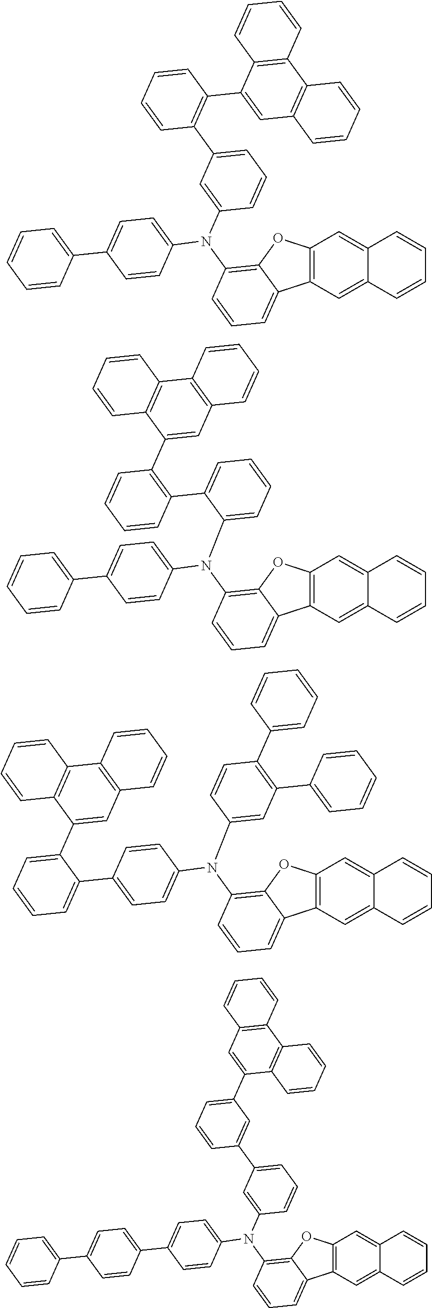

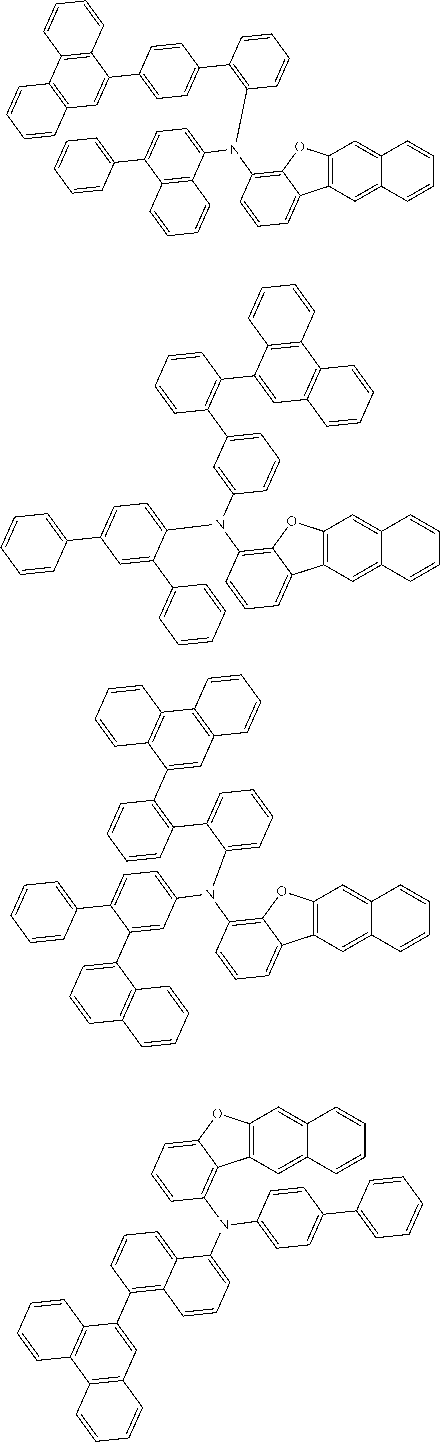

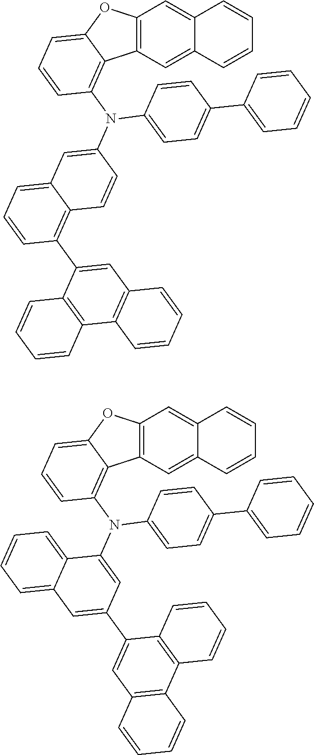 [SMILES: c1ccc(-c2ccc(N(c3ccc4c(-c5cc6ccccc6c6ccccc56)cccc4c3)c3cccc4oc5cc6ccccc6cc5c34)cc2)cc1 ; c1ccc(-c2ccc(N(c3cc(-c4cc5ccccc5c5ccccc45)cc4ccccc34)c3cccc4oc5cc6ccccc6cc5c34)cc2)cc1]

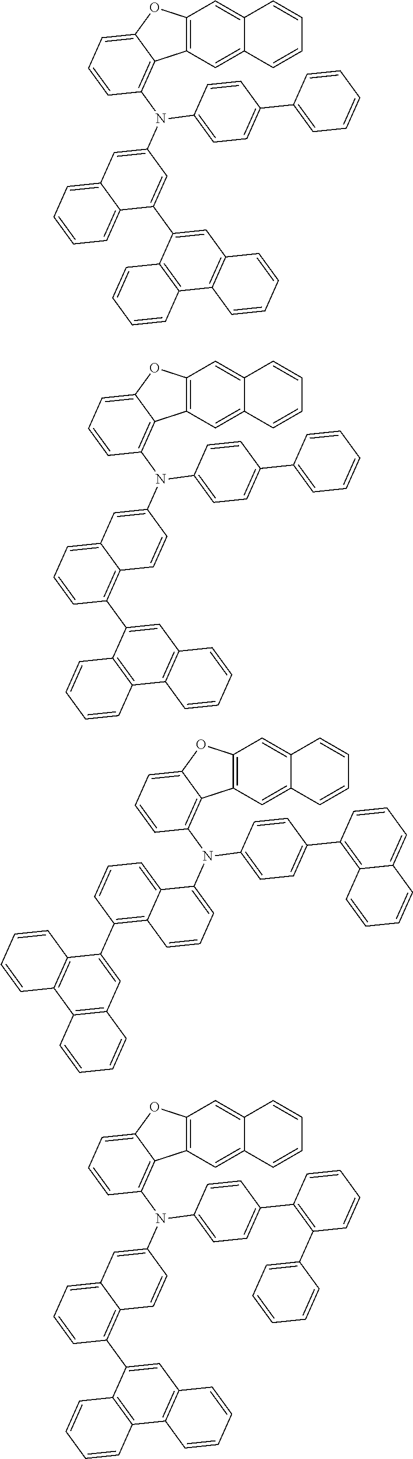

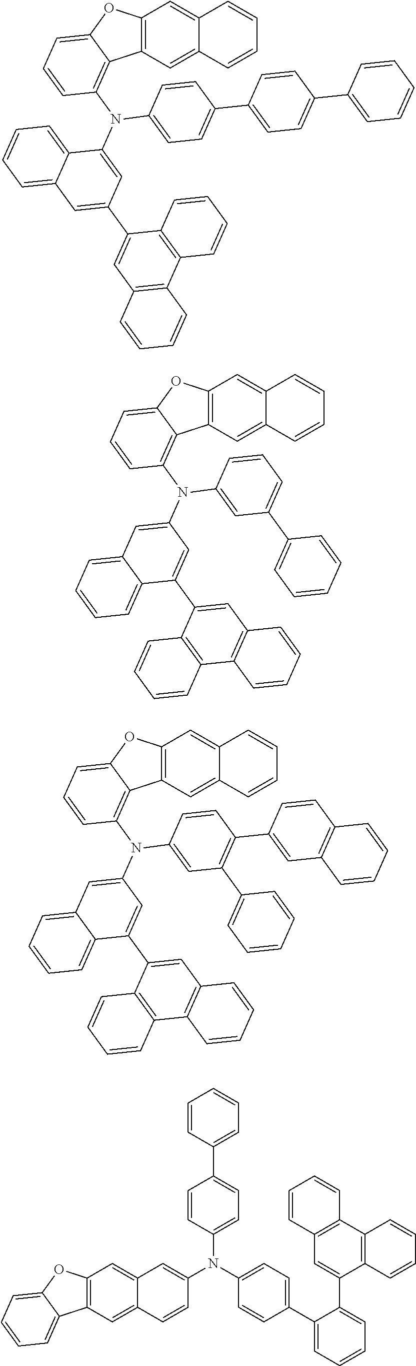

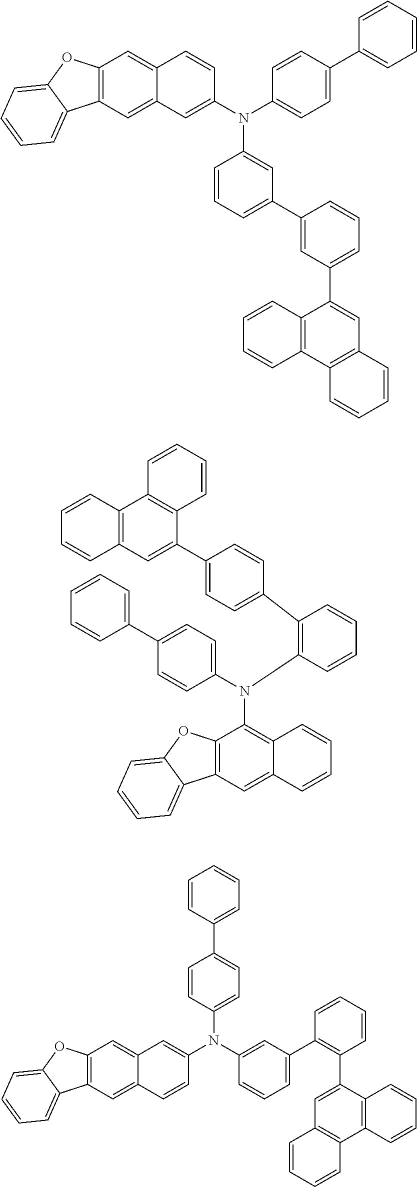 [SMILES: c1ccc(-c2ccc(N(c3cccc(-c4cccc(-c5cc6ccccc6c6ccccc56)c4)c3)c3ccc4cc5oc6ccccc6c5cc4c3)cc2)cc1 ; c1ccc(-c2ccc(N(c3ccccc3-c3ccc(-c4cc5ccccc5c5ccccc45)cc3)c3c4ccccc4cc4c3oc3ccccc34)cc2)cc1 ; c1ccc(-c2ccc(N(c3cccc(-c4ccccc4-c4cc5ccccc5c5ccccc45)c3)c3ccc4cc5c(cc4c3)oc3ccccc35)cc2)cc1]

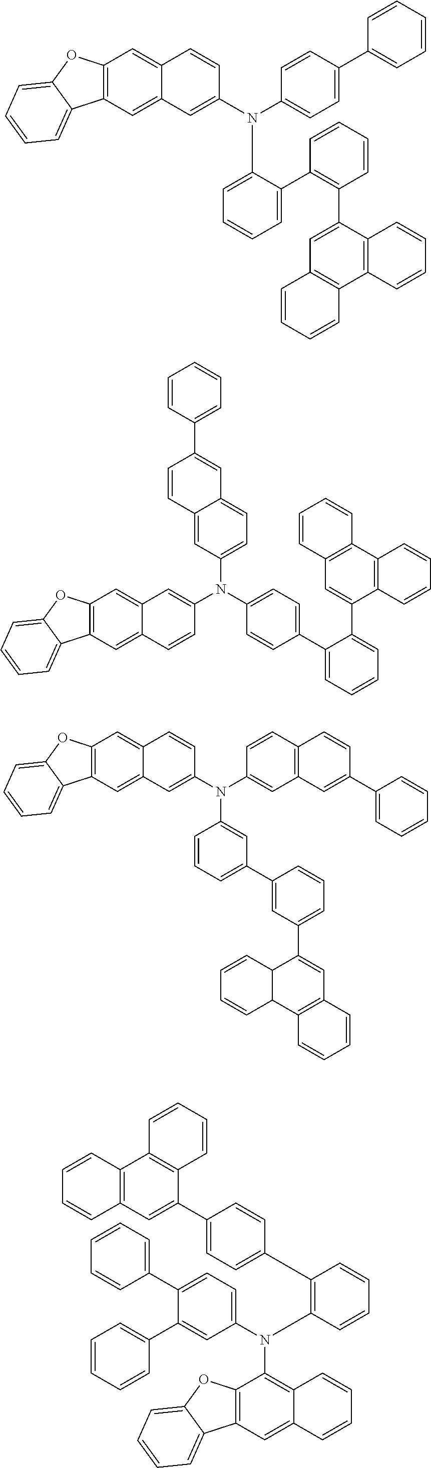 [SMILES: c1ccc(-c2ccc(N(c3ccc4cc5oc6ccccc6c5cc4c3)c3ccccc3-c3ccccc3-c3cc4ccccc4c4ccccc34)cc2)cc1 ; c1ccc(-c2ccc3cc(N(c4ccc(-c5ccccc5-c5cc6ccccc6c6ccccc56)cc4)c4ccc5cc6c(cc5c4)oc4ccccc46)ccc3c2)cc1 ; C1=CC2C(c3cccc(-c4cccc(N(c5ccc6ccc(-c7ccccc7)cc6c5)c5ccc6cc7oc8ccccc8c7cc6c5)c4)c3)=Cc3ccccc3C2C=C1 ; c1ccc(-c2ccc(N(c3ccccc3-c3ccc(-c4cc5ccccc5c5ccccc45)cc3)c3c4ccccc4cc4c3oc3ccccc34)cc2-c2ccccc2)cc1]

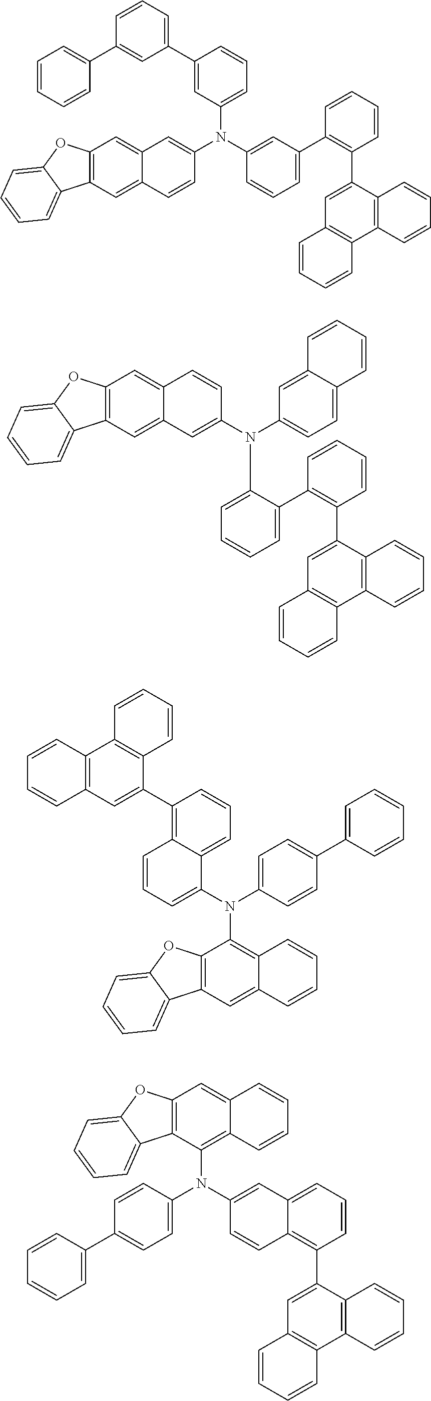

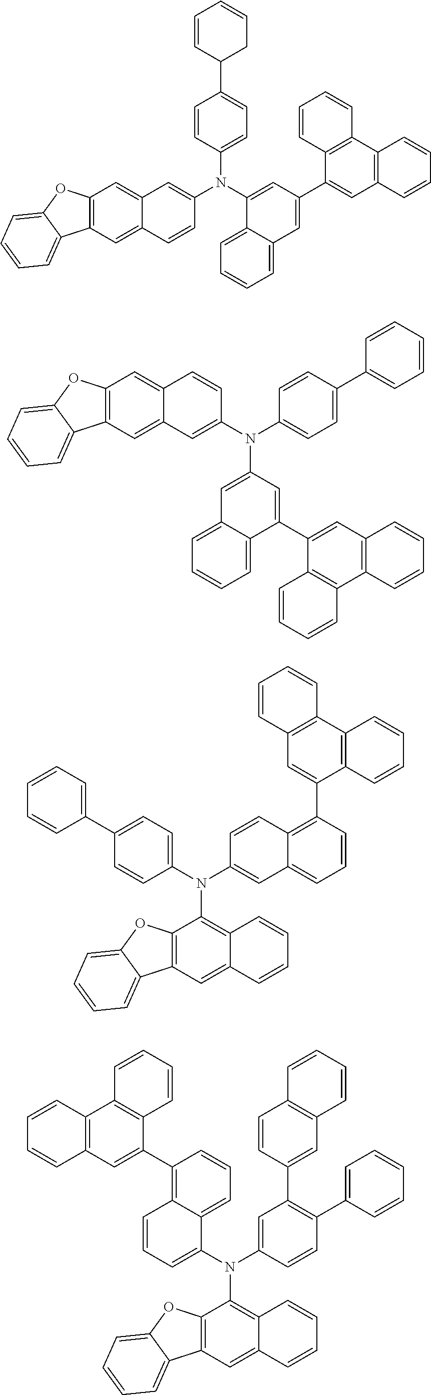

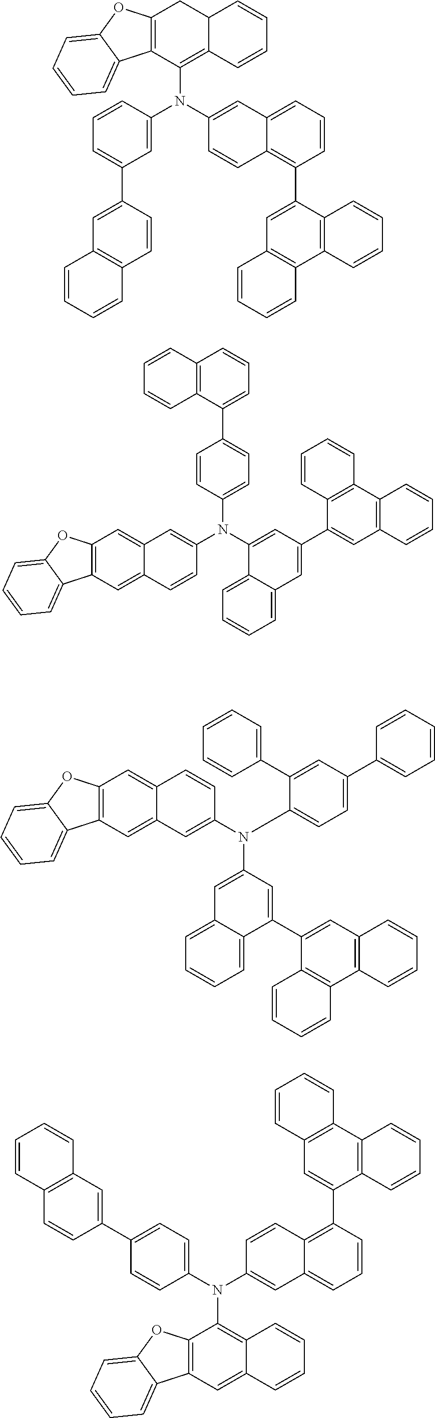

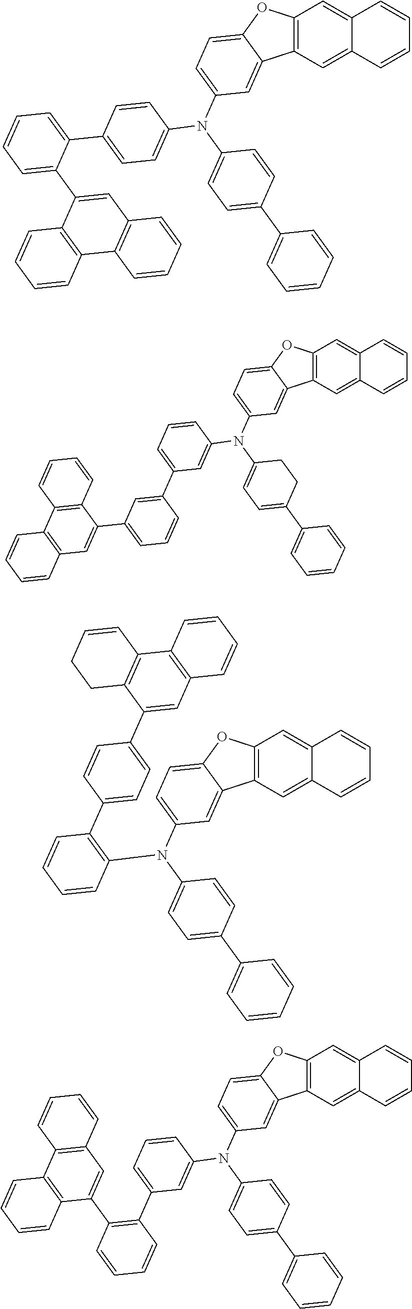

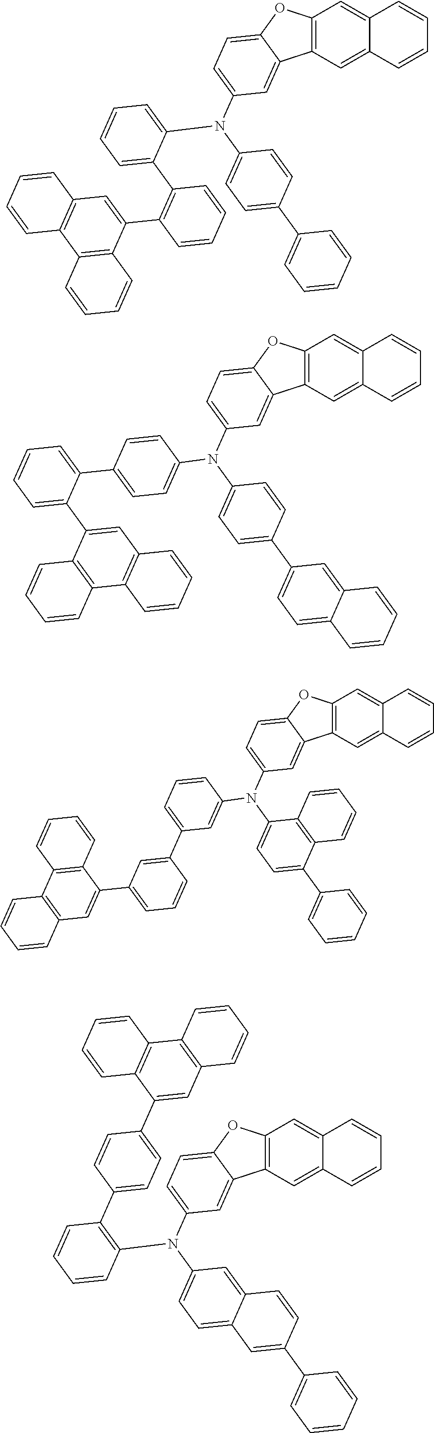

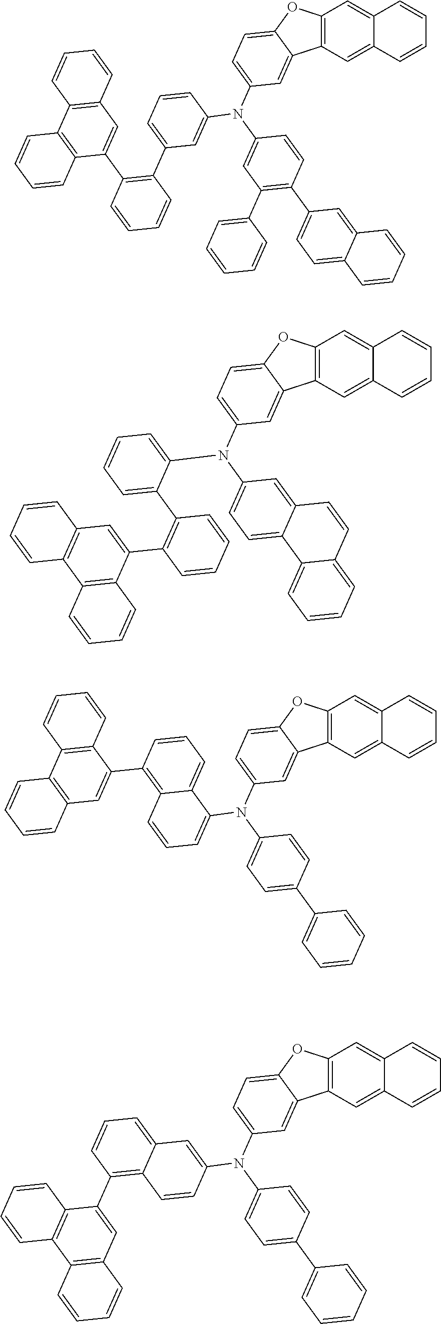 [SMILES: c1ccc(-c2cc(N(c3cccc(-c4ccccc4-c4cc5ccccc5c5ccccc45)c3)c3ccc4oc5cc6ccccc6cc5c4c3)ccc2-c2ccc3ccccc3c2)cc1 ; c1ccc(-c2cc3ccccc3c3ccccc23)c(-c2ccccc2N(c2ccc3c(ccc4ccccc43)c2)c2ccc3oc4cc5ccccc5cc4c3c2)c1 ; c1ccc(-c2ccc(N(c3ccc4oc5cc6ccccc6cc5c4c3)c3cccc4c(-c5cc6ccccc6c6ccccc56)cccc34)cc2)cc1 ; c1ccc(-c2ccc(N(c3ccc4c(-c5cc6ccccc6c6ccccc56)cccc4c3)c3ccc4oc5cc6ccccc6cc5c4c3)cc2)cc1]

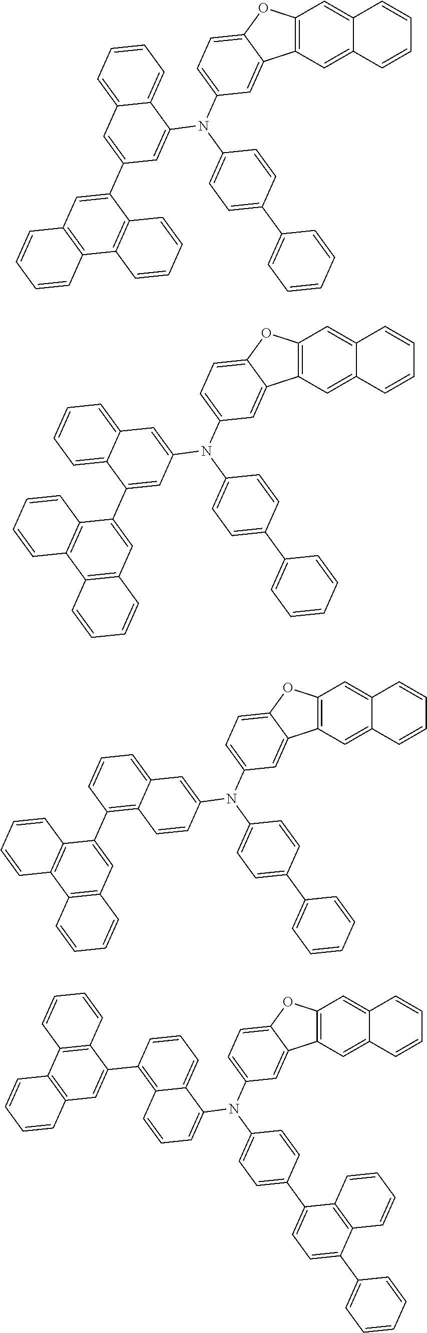

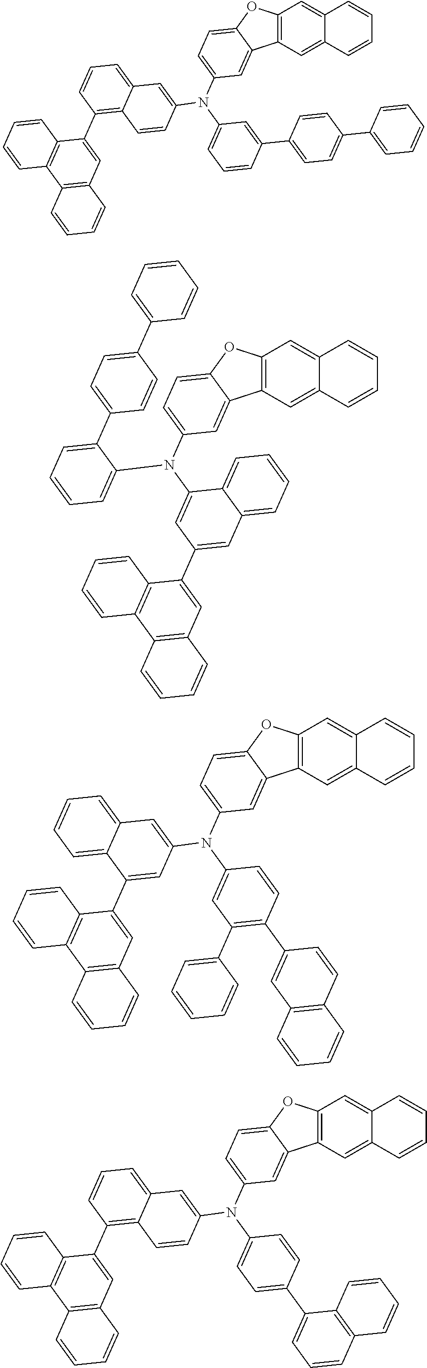

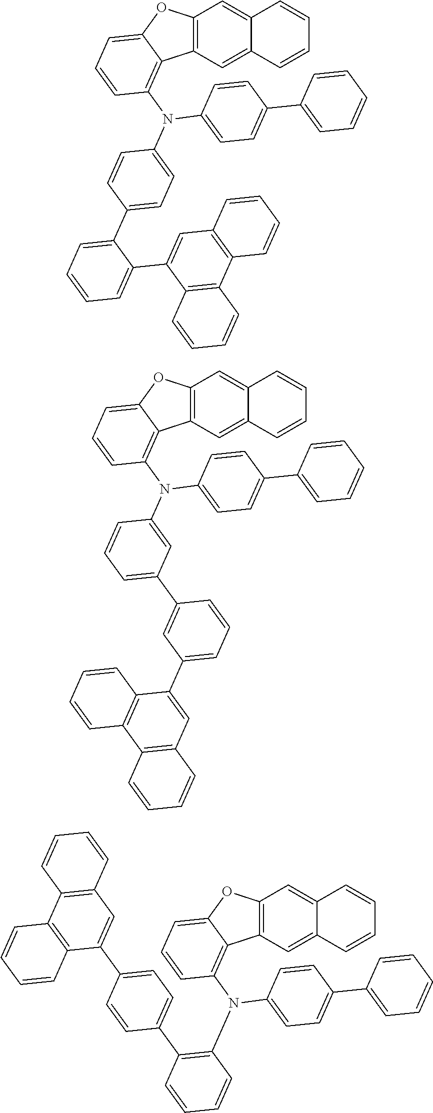

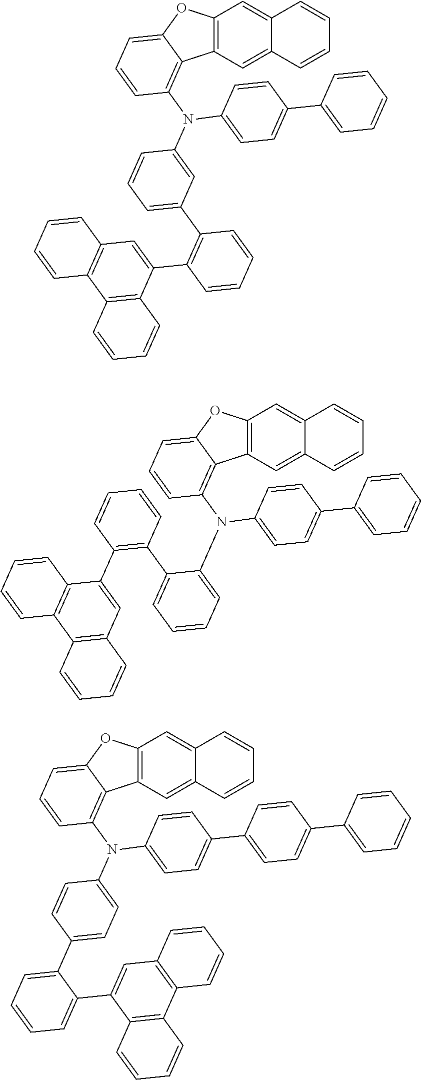

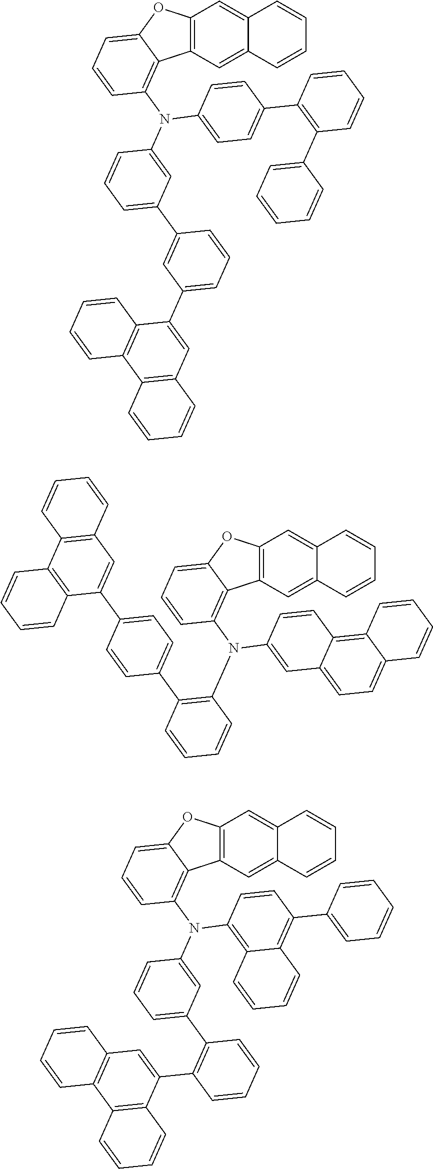 [SMILES: c1ccc(-c2ccccc2-c2ccc(N(c3cccc(-c4cccc(-c5cc6ccccc6c6ccccc56)c4)c3)c3cccc4oc5cc6ccccc6cc5c34)cc2)cc1 ; c1ccc(N(c2ccc3c(ccc4ccccc43)c2)c2cccc3oc4cc5ccccc5cc4c23)c(-c2ccc(-c3cc4ccccc4c4ccccc34)cc2)c1 ; c1ccc(-c2ccc(N(c3cccc(-c4ccccc4-c4cc5ccccc5c5ccccc45)c3)c3cccc4oc5cc6ccccc6cc5c34)c3ccccc23)cc1]

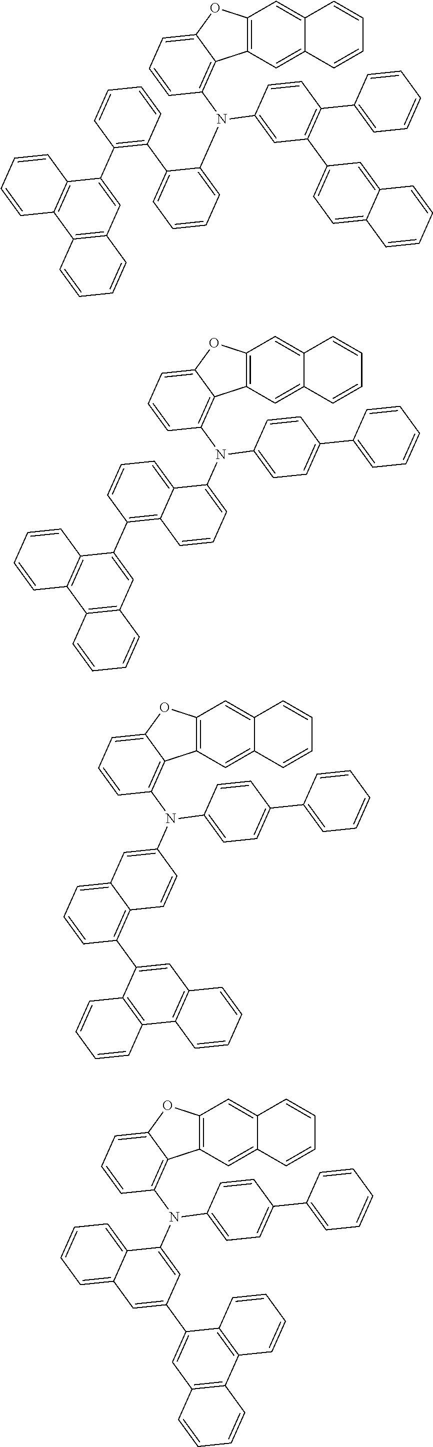 [SMILES: c1ccc(-c2ccc(N(c3ccccc3-c3ccccc3-c3cc4ccccc4c4ccccc34)c3cccc4oc5cc6ccccc6cc5c34)cc2-c2ccc3ccccc3c2)cc1 ; c1ccc(-c2ccc(N(c3cccc4c(-c5cc6ccccc6c6ccccc56)cccc34)c3cccc4oc5cc6ccccc6cc5c34)cc2)cc1 ; c1ccc(-c2ccc(N(c3ccc4c(-c5cc6ccccc6c6ccccc56)cccc4c3)c3cccc4oc5cc6ccccc6cc5c34)cc2)cc1 ; c1ccc(-c2ccc(N(c3cc(-c4cc5ccccc5c5ccccc45)cc4ccccc34)c3cccc4oc5cc6ccccc6cc5c34)cc2)cc1]

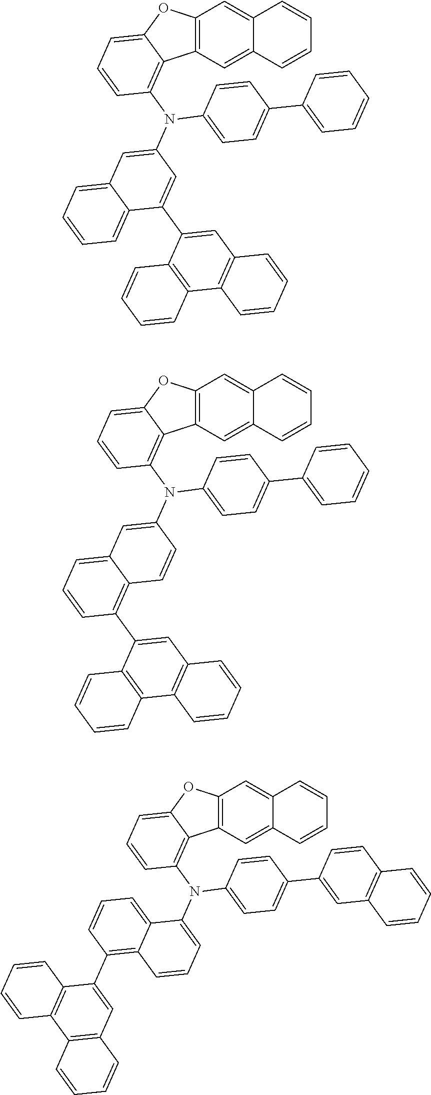 [SMILES: c1ccc(-c2ccc(N(c3cc(-c4cc5ccccc5c5ccccc45)c4ccccc4c3)c3cccc4oc5cc6ccccc6cc5c34)cc2)cc1 ; c1ccc(-c2ccc(N(c3ccc4c(-c5cc6ccccc6c6ccccc56)cccc4c3)c3cccc4oc5cc6ccccc6cc5c34)cc2)cc1 ; c1ccc2cc(-c3ccc(N(c4cccc5c(-c6cc7ccccc7c7ccccc67)cccc45)c4cccc5oc6cc7ccccc7cc6c45)cc3)ccc2c1]

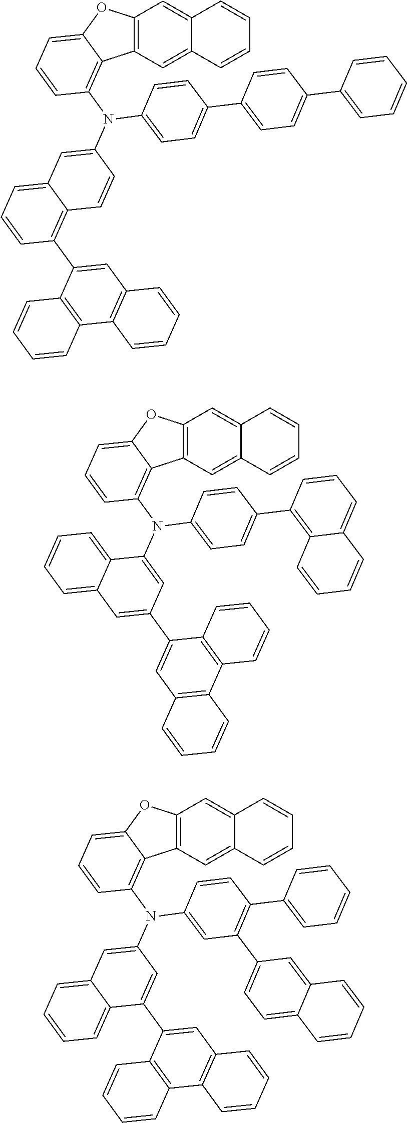 [SMILES: c1ccc(-c2ccc(-c3ccc(N(c4ccc5c(-c6cc7ccccc7c7ccccc67)cccc5c4)c4cccc5oc6cc7ccccc7cc6c45)cc3)cc2)cc1 ; c1ccc2cc3c(cc2c1)oc1cccc(N(c2ccc(-c4cccc5ccccc45)cc2)c2cc(-c4cc5ccccc5c5ccccc45)cc4ccccc24)c13 ; c1ccc(-c2ccc(N(c3cc(-c4cc5ccccc5c5ccccc45)c4ccccc4c3)c3cccc4oc5cc6ccccc6cc5c34)cc2-c2ccc3ccccc3c2)cc1]

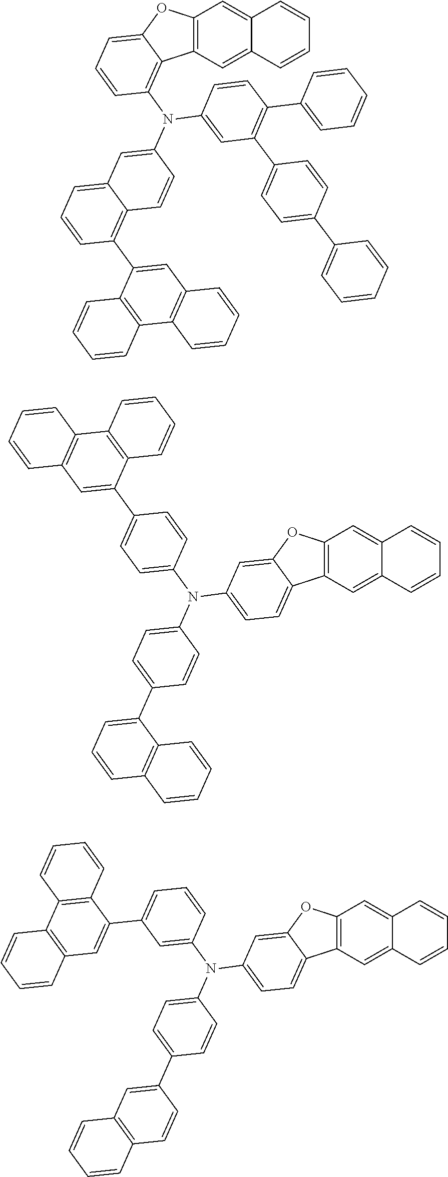

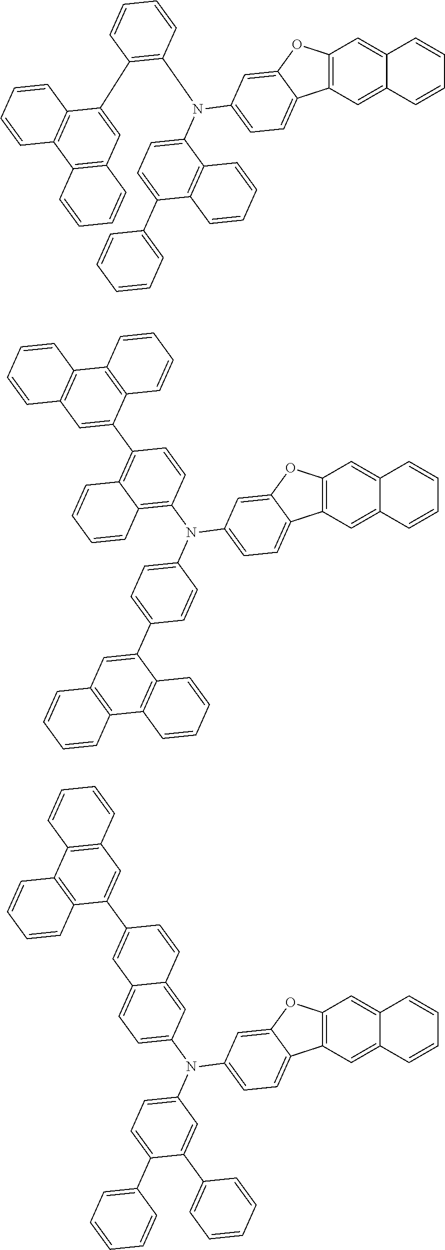

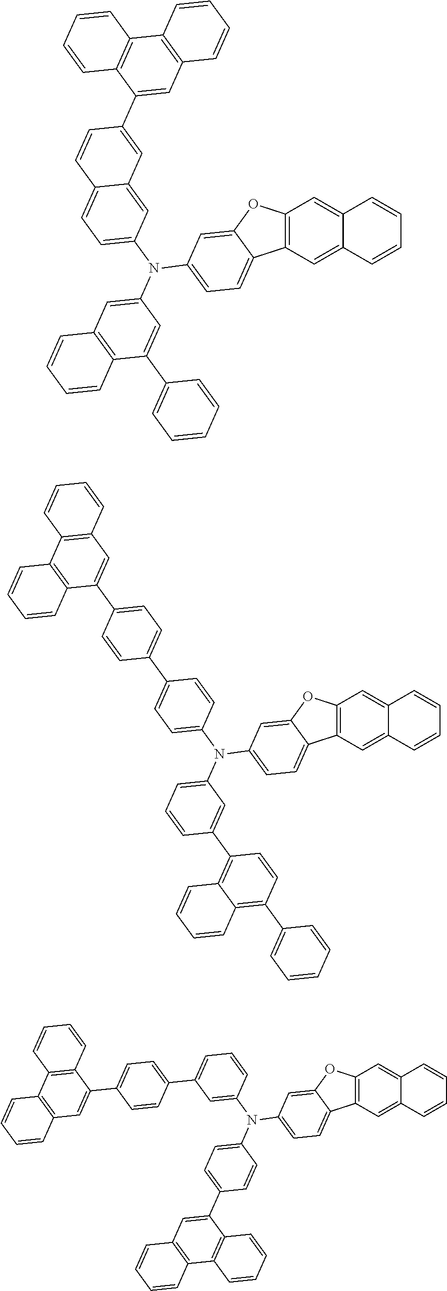 [SMILES: c1ccc(-c2cc(N(c3ccc4ccc(-c5cc6ccccc6c6ccccc56)cc4c3)c3ccc4c(c3)oc3cc5ccccc5cc34)cc3ccccc23)cc1 ; c1ccc(-c2ccc(-c3cccc(N(c4ccc(-c5ccc(-c6cc7ccccc7c7ccccc67)cc5)cc4)c4ccc5c(c4)oc4cc6ccccc6cc45)c3)c3ccccc23)cc1 ; c1cc(-c2ccc(-c3cc4ccccc4c4ccccc34)cc2)cc(N(c2ccc(-c3cc4ccccc4c4ccccc34)cc2)c2ccc3c(c2)oc2cc4ccccc4cc23)c1]

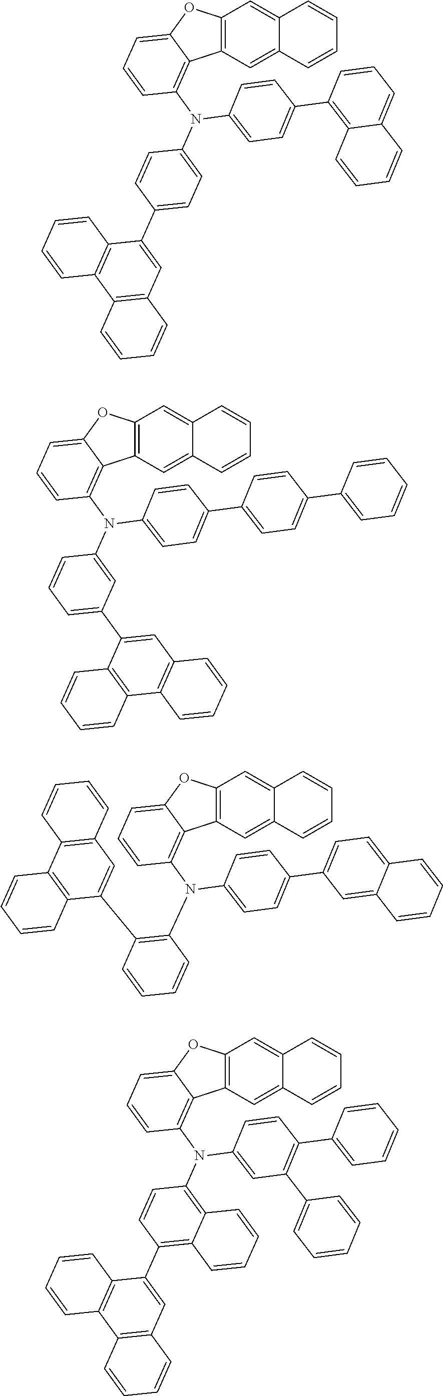 [SMILES: c1ccc2cc3c(cc2c1)oc1cccc(N(c2ccc(-c4cccc5ccccc45)cc2)c2ccc(-c4cc5ccccc5c5ccccc45)cc2)c13 ; c1ccc(-c2ccc(-c3ccc(N(c4cccc(-c5cc6ccccc6c6ccccc56)c4)c4cccc5oc6cc7ccccc7cc6c45)cc3)cc2)cc1 ; c1ccc(N(c2ccc(-c3ccc4ccccc4c3)cc2)c2cccc3oc4cc5ccccc5cc4c23)c(-c2cc3ccccc3c3ccccc23)c1 ; c1ccc(-c2ccc(N(c3ccc(-c4cc5ccccc5c5ccccc45)c4ccccc34)c3cccc4oc5cc6ccccc6cc5c34)cc2-c2ccccc2)cc1]

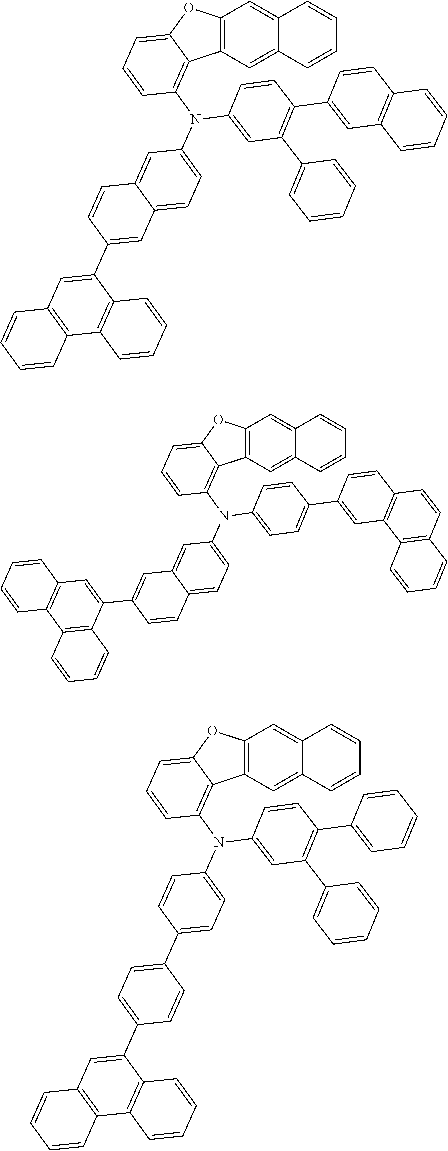 [SMILES: c1ccc(-c2cc(N(c3ccc4cc(-c5cc6ccccc6c6ccccc56)ccc4c3)c3cccc4oc5cc6ccccc6cc5c34)ccc2-c2ccc3ccccc3c2)cc1 ; c1ccc2cc3c(cc2c1)oc1cccc(N(c2ccc(-c4ccc5ccc6ccccc6c5c4)cc2)c2ccc4ccc(-c5cc6ccccc6c6ccccc56)cc4c2)c13 ; c1ccc(-c2ccc(N(c3ccc(-c4ccc(-c5cc6ccccc6c6ccccc56)cc4)cc3)c3cccc4oc5cc6ccccc6cc5c34)cc2-c2ccccc2)cc1]

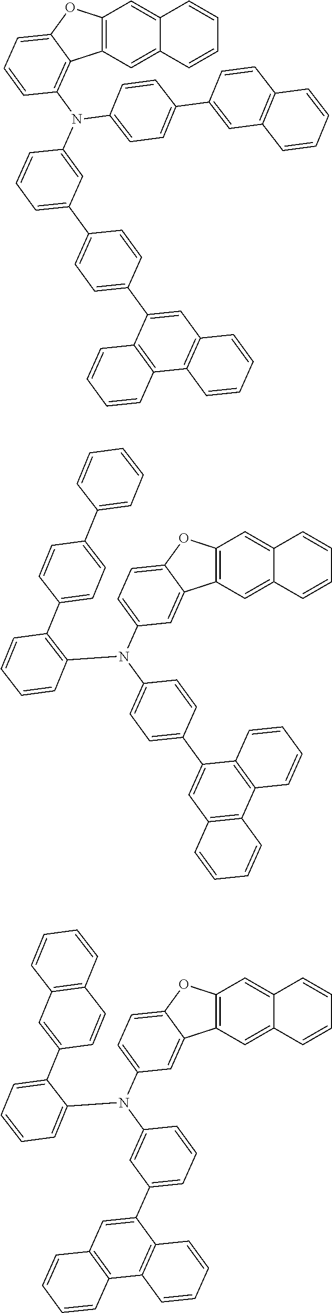

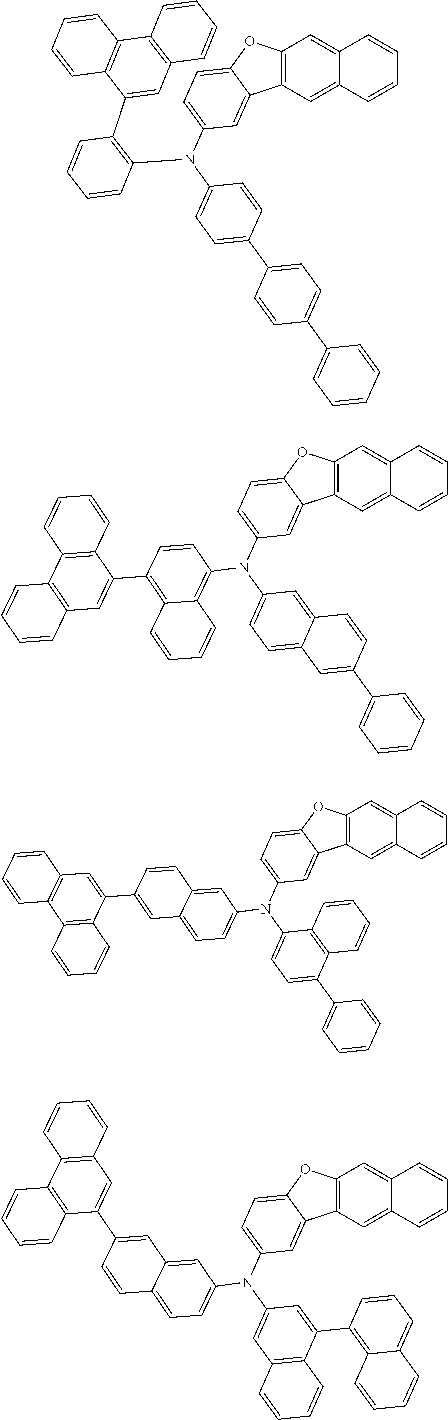

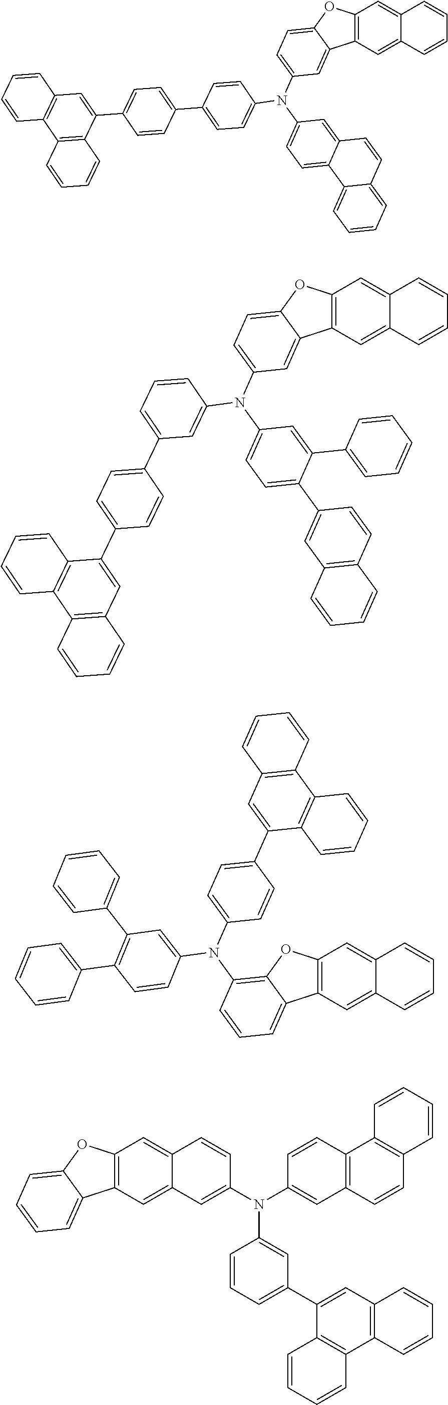 [SMILES: c1ccc2cc3c(cc2c1)oc1ccc(N(c2ccc(-c4ccc(-c5cc6ccccc6c6ccccc56)cc4)cc2)c2ccc4c(ccc5ccccc54)c2)cc13 ; c1ccc(-c2cc(N(c3cccc(-c4ccc(-c5cc6ccccc6c6ccccc56)cc4)c3)c3ccc4oc5cc6ccccc6cc5c4c3)ccc2-c2ccc3ccccc3c2)cc1 ; c1ccc(-c2ccc(N(c3ccc(-c4cc5ccccc5c5ccccc45)cc3)c3cccc4c3oc3cc5ccccc5cc34)cc2-c2ccccc2)cc1 ; c1cc(-c2cc3ccccc3c3ccccc23)cc(N(c2ccc3cc4oc5ccccc5c4cc3c2)c2ccc3c(ccc4ccccc43)c2)c1]

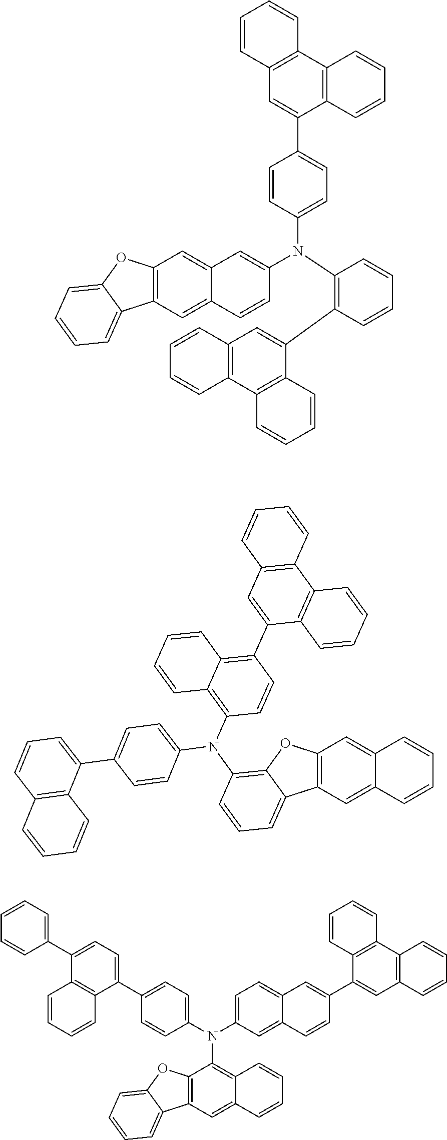 [SMILES: c1ccc(N(c2ccc(-c3cc4ccccc4c4ccccc34)cc2)c2ccc3cc4c(cc3c2)oc2ccccc24)c(-c2cc3ccccc3c3ccccc23)c1 ; c1ccc2cc3c(cc2c1)oc1c(N(c2ccc(-c4cccc5ccccc45)cc2)c2ccc(-c4cc5ccccc5c5ccccc45)c4ccccc24)cccc13 ; c1ccc(-c2ccc(-c3ccc(N(c4ccc5cc(-c6cc7ccccc7c7ccccc67)ccc5c4)c4c5ccccc5cc5c4oc4ccccc45)cc3)c3ccccc23)cc1]

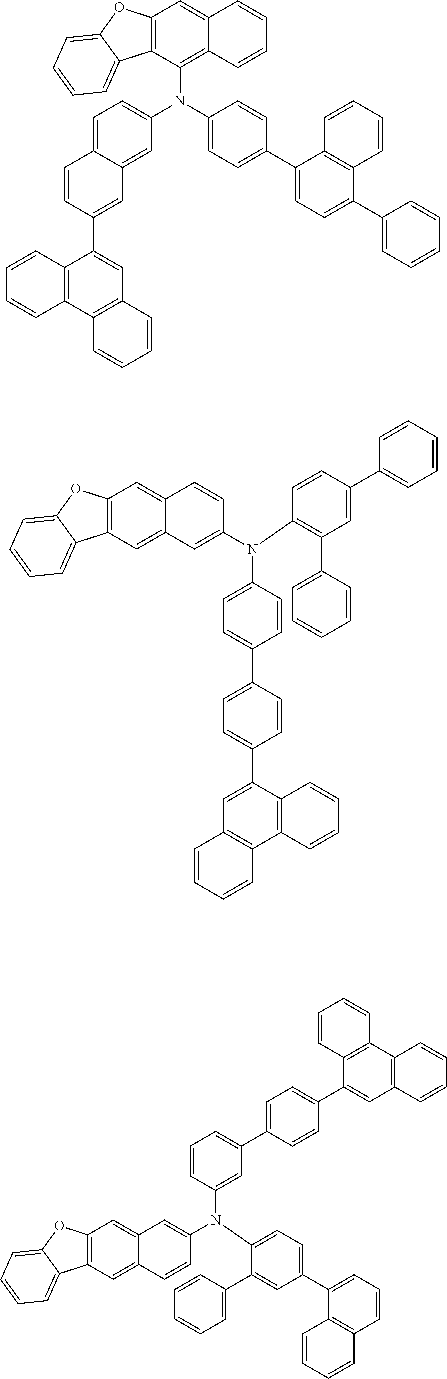 [SMILES: c1ccc(-c2ccc(-c3ccc(N(c4ccc5ccc(-c6cc7ccccc7c7ccccc67)cc5c4)c4c5ccccc5cc5oc6ccccc6c45)cc3)c3ccccc23)cc1 ; c1ccc(-c2ccc(N(c3ccc(-c4ccc(-c5cc6ccccc6c6ccccc56)cc4)cc3)c3ccc4cc5oc6ccccc6c5cc4c3)c(-c3ccccc3)c2)cc1 ; c1ccc(-c2cc(-c3cccc4ccccc34)ccc2N(c2cccc(-c3ccc(-c4cc5ccccc5c5ccccc45)cc3)c2)c2ccc3cc4c(cc3c2)oc2ccccc24)cc1]

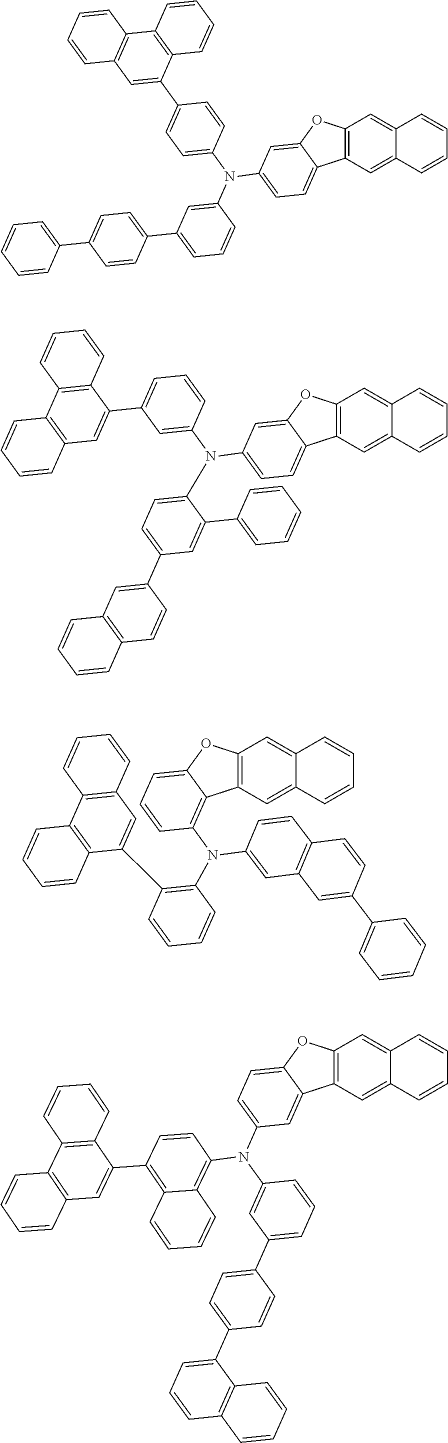 [SMILES: c1ccc(-c2ccc(-c3cccc(N(c4ccc(-c5cc6ccccc6c6ccccc56)cc4)c4ccc5c(c4)oc4cc6ccccc6cc45)c3)cc2)cc1 ; c1ccc(-c2cc(-c3ccc4ccccc4c3)ccc2N(c2cccc(-c3cc4ccccc4c4ccccc34)c2)c2ccc3c(c2)oc2cc4ccccc4cc23)cc1 ; c1ccc(-c2ccc3ccc(N(c4ccccc4-c4cc5ccccc5c5ccccc45)c4cccc5oc6cc7ccccc7cc6c45)cc3c2)cc1 ; c1cc(-c2ccc(-c3cccc4ccccc34)cc2)cc(N(c2ccc3oc4cc5ccccc5cc4c3c2)c2ccc(-c3cc4ccccc4c4ccccc34)c3ccccc23)c1]

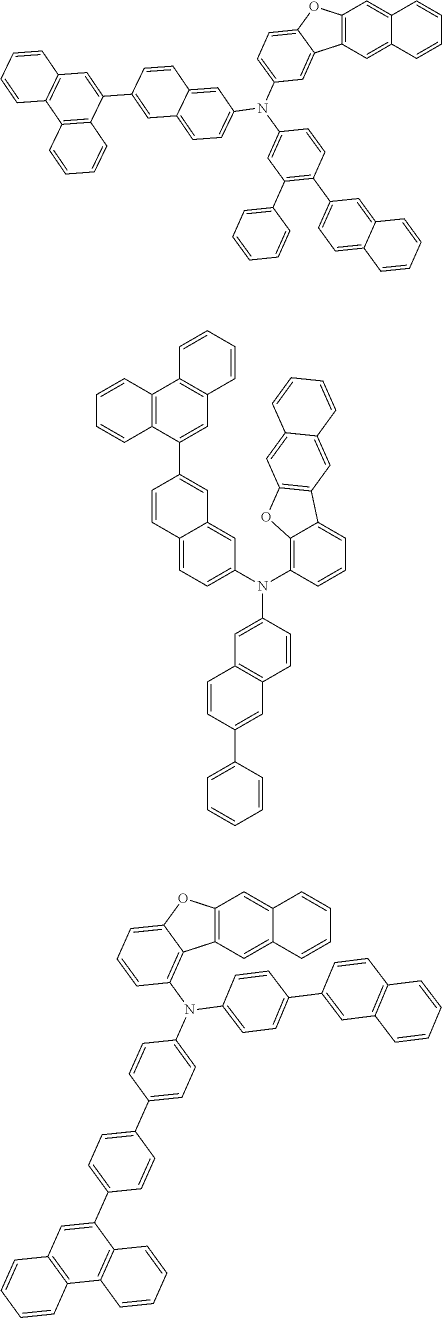 [SMILES: c1ccc(-c2cc(N(c3ccc4cc(-c5cc6ccccc6c6ccccc56)ccc4c3)c3ccc4oc5cc6ccccc6cc5c4c3)ccc2-c2ccc3ccccc3c2)cc1 ; c1ccc(-c2ccc3cc(N(c4ccc5ccc(-c6cc7ccccc7c7ccccc67)cc5c4)c4cccc5c4oc4cc6ccccc6cc45)ccc3c2)cc1 ; c1ccc2cc(-c3ccc(N(c4ccc(-c5ccc(-c6cc7ccccc7c7ccccc67)cc5)cc4)c4cccc5oc6cc7ccccc7cc6c45)cc3)ccc2c1]

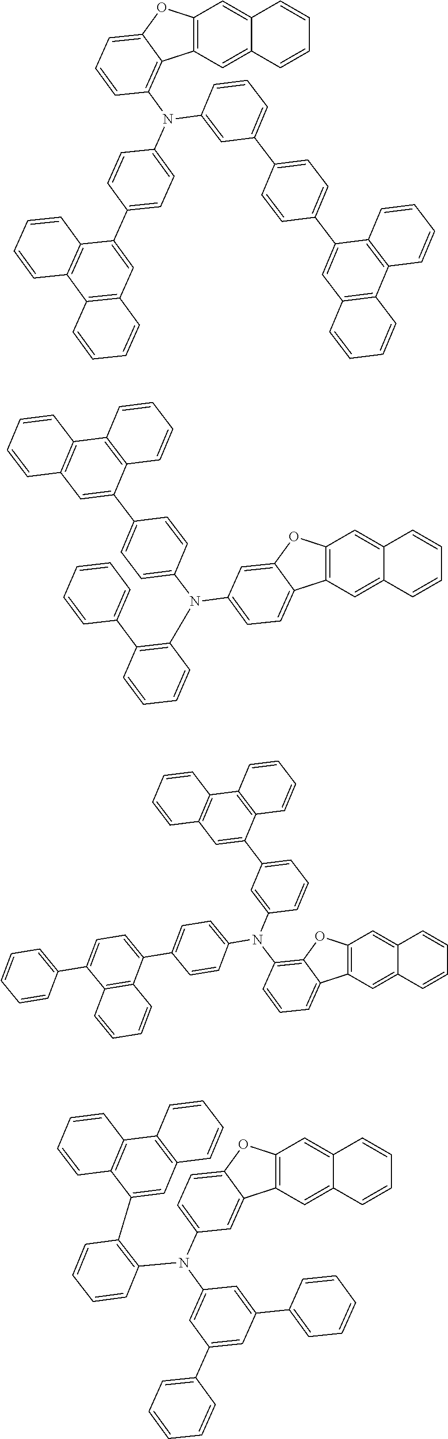

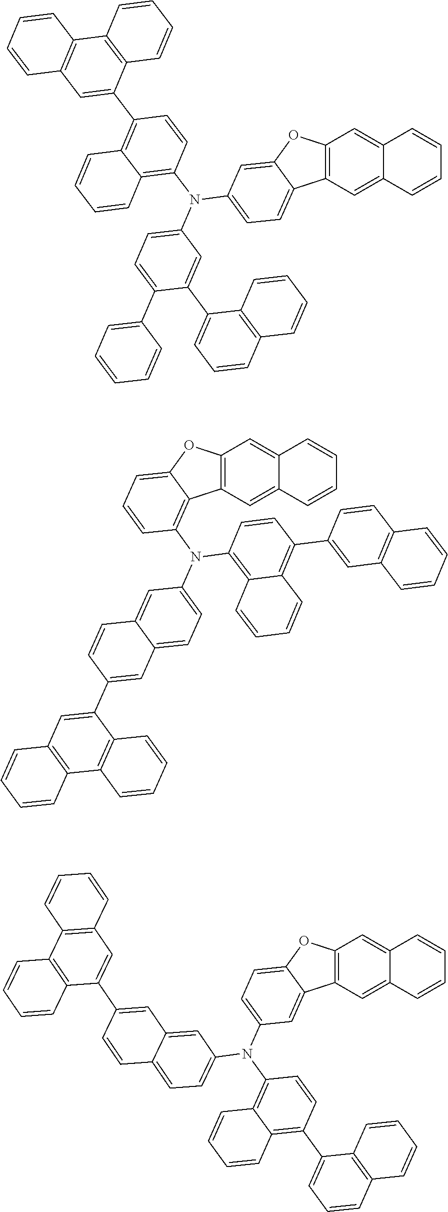 [SMILES: c1ccc(-c2ccc(N(c3ccc4c(c3)oc3cc5ccccc5cc34)c3ccc(-c4cc5ccccc5c5ccccc45)c4ccccc34)cc2-c2cccc3ccccc23)cc1 ; c1ccc2cc(-c3ccc(N(c4ccc5cc(-c6cc7ccccc7c7ccccc67)ccc5c4)c4cccc5oc6cc7ccccc7cc6c45)c4ccccc34)ccc2c1 ; c1ccc2cc3c(cc2c1)oc1ccc(N(c2ccc4ccc(-c5cc6ccccc6c6ccccc56)cc4c2)c2ccc(-c4cccc5ccccc45)c4ccccc24)cc13]

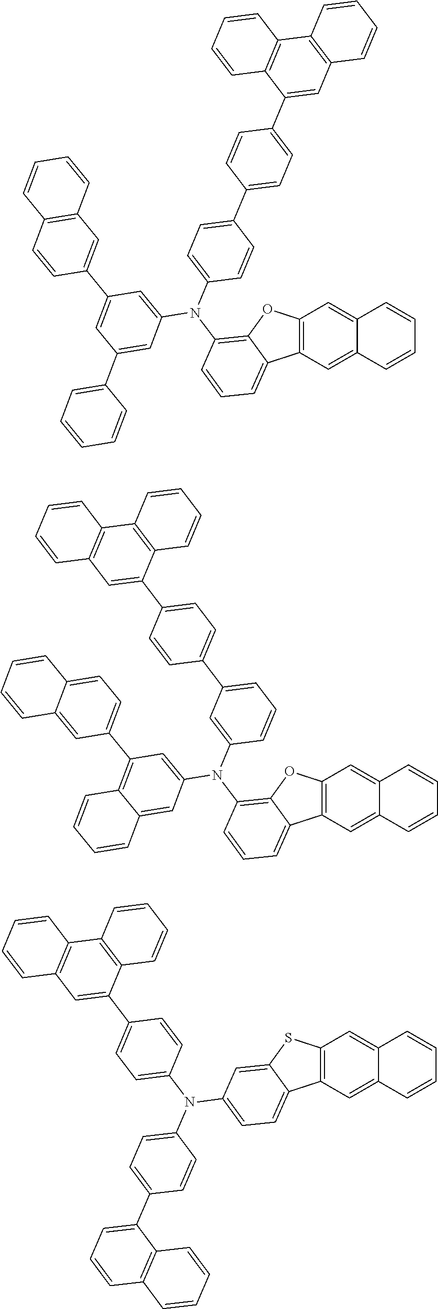 [SMILES: c1ccc(-c2cc(-c3ccc4ccccc4c3)cc(N(c3ccc(-c4ccc(-c5cc6ccccc6c6ccccc56)cc4)cc3)c3cccc4c3oc3cc5ccccc5cc34)c2)cc1 ; c1cc(-c2ccc(-c3cc4ccccc4c4ccccc34)cc2)cc(N(c2cc(-c3ccc4ccccc4c3)c3ccccc3c2)c2cccc3c2oc2cc4ccccc4cc23)c1 ; c1ccc2cc3c(cc2c1)sc1cc(N(c2ccc(-c4cccc5ccccc45)cc2)c2ccc(-c4cc5ccccc5c5ccccc45)cc2)ccc13]

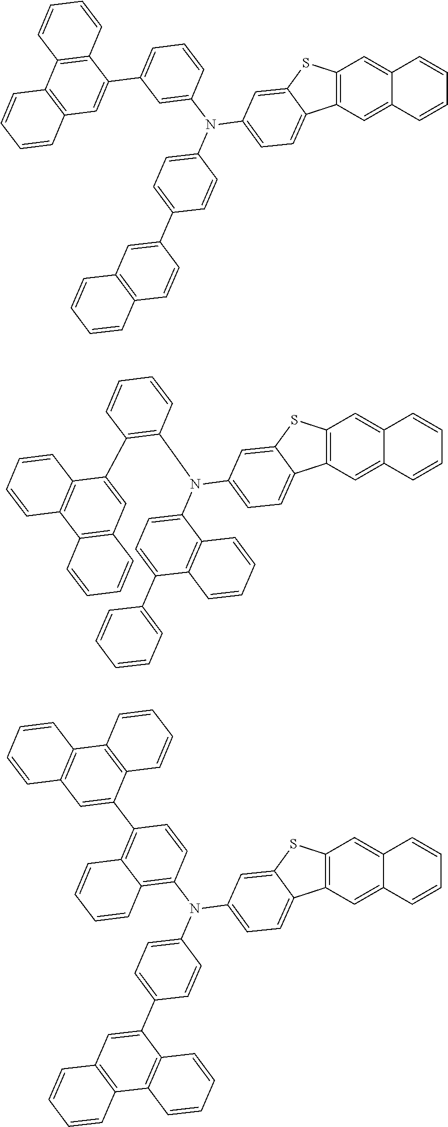

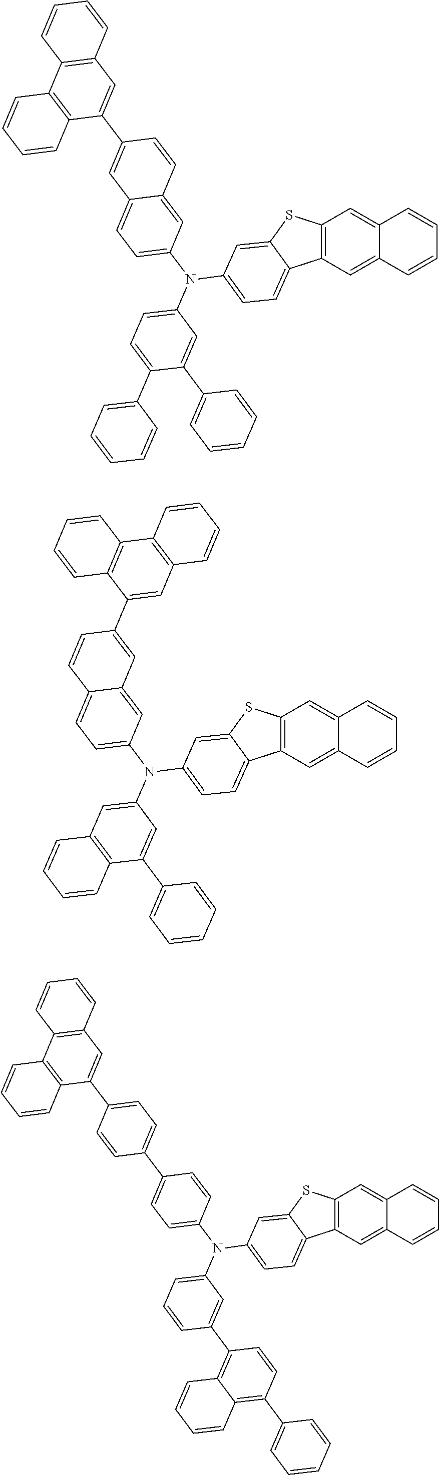 [SMILES: c1ccc(-c2ccc(N(c3ccc4cc(-c5cc6ccccc6c6ccccc56)ccc4c3)c3ccc4c(c3)sc3cc5ccccc5cc34)cc2-c2ccccc2)cc1 ; c1ccc(-c2cc(N(c3ccc4ccc(-c5cc6ccccc6c6ccccc56)cc4c3)c3ccc4c(c3)sc3cc5ccccc5cc34)cc3ccccc23)cc1 ; c1ccc(-c2ccc(-c3cccc(N(c4ccc(-c5ccc(-c6cc7ccccc7c7ccccc67)cc5)cc4)c4ccc5c(c4)sc4cc6ccccc6cc45)c3)c3ccccc23)cc1]

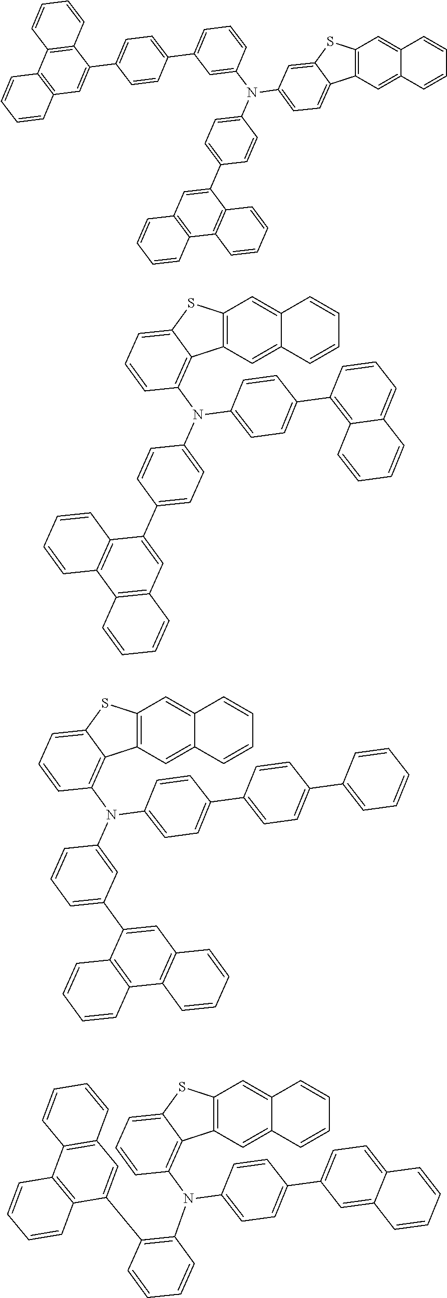

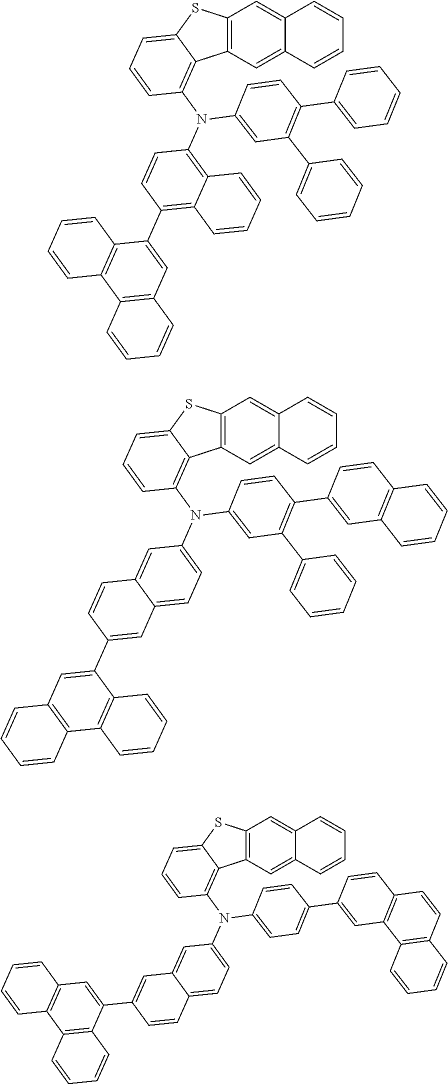 [SMILES: c1ccc(-c2ccc(N(c3ccc(-c4cc5ccccc5c5ccccc45)c4ccccc34)c3cccc4sc5cc6ccccc6cc5c34)cc2-c2ccccc2)cc1 ; c1ccc(-c2cc(N(c3ccc4cc(-c5cc6ccccc6c6ccccc56)ccc4c3)c3cccc4sc5cc6ccccc6cc5c34)ccc2-c2ccc3ccccc3c2)cc1 ; c1ccc2cc3c(cc2c1)sc1cccc(N(c2ccc(-c4ccc5ccc6ccccc6c5c4)cc2)c2ccc4ccc(-c5cc6ccccc6c6ccccc56)cc4c2)c13]

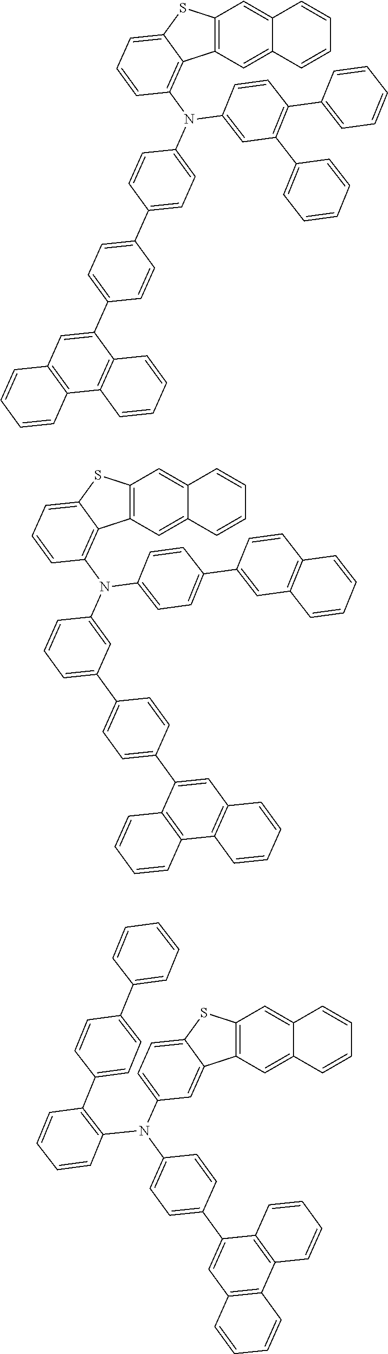 [SMILES: c1ccc(-c2ccc(N(c3ccc(-c4ccc(-c5cc6ccccc6c6ccccc56)cc4)cc3)c3cccc4sc5cc6ccccc6cc5c34)cc2-c2ccccc2)cc1 ; c1cc(-c2ccc(-c3cc4ccccc4c4ccccc34)cc2)cc(N(c2ccc(-c3ccc4ccccc4c3)cc2)c2cccc3sc4cc5ccccc5cc4c23)c1 ; c1ccc(-c2ccc(-c3ccccc3N(c3ccc(-c4cc5ccccc5c5ccccc45)cc3)c3ccc4sc5cc6ccccc6cc5c4c3)cc2)cc1]

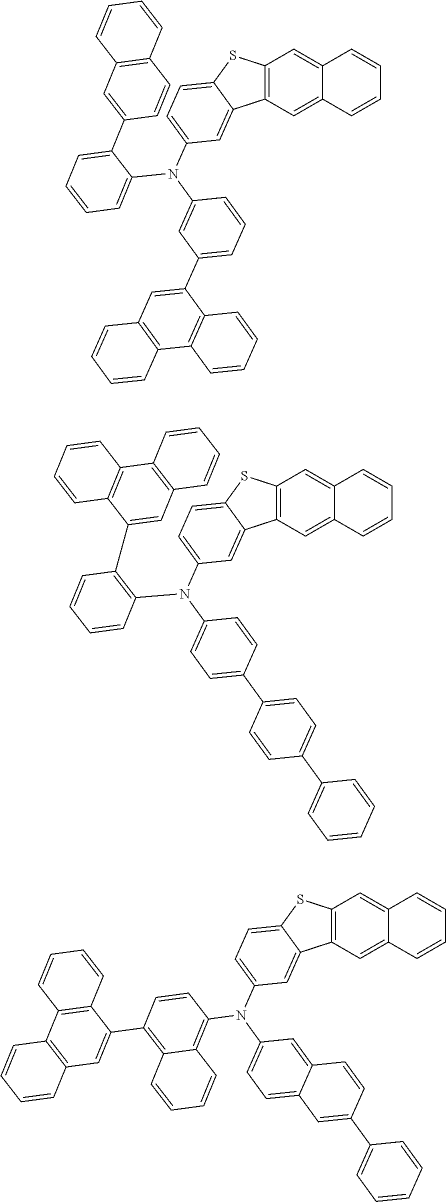 [SMILES: c1cc(-c2cc3ccccc3c3ccccc23)cc(N(c2ccc3sc4cc5ccccc5cc4c3c2)c2ccccc2-c2ccc3ccccc3c2)c1 ; c1ccc(-c2ccc(-c3ccc(N(c4ccc5sc6cc7ccccc7cc6c5c4)c4ccccc4-c4cc5ccccc5c5ccccc45)cc3)cc2)cc1 ; c1ccc(-c2ccc3cc(N(c4ccc5sc6cc7ccccc7cc6c5c4)c4ccc(-c5cc6ccccc6c6ccccc56)c5ccccc45)ccc3c2)cc1]

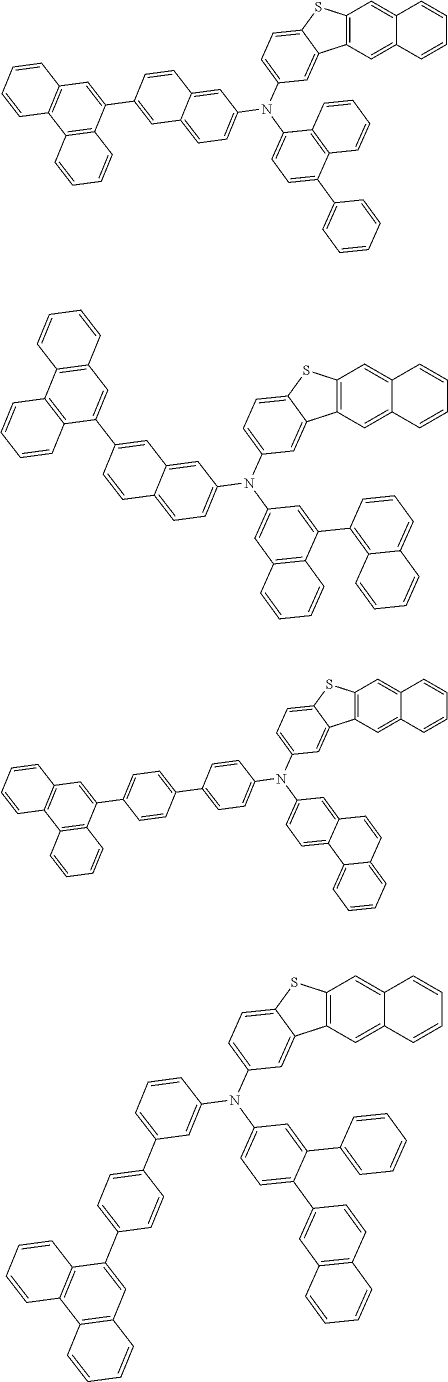 [SMILES: c1ccc(-c2ccc(N(c3ccc4cc(-c5cc6ccccc6c6ccccc56)ccc4c3)c3ccc4sc5cc6ccccc6cc5c4c3)c3ccccc23)cc1 ; c1ccc2cc3c(cc2c1)sc1ccc(N(c2ccc4ccc(-c5cc6ccccc6c6ccccc56)cc4c2)c2cc(-c4cccc5ccccc45)c4ccccc4c2)cc13 ; c1ccc2cc3c(cc2c1)sc1ccc(N(c2ccc(-c4ccc(-c5cc6ccccc6c6ccccc56)cc4)cc2)c2ccc4c(ccc5ccccc54)c2)cc13 ; c1ccc(-c2cc(N(c3cccc(-c4ccc(-c5cc6ccccc6c6ccccc56)cc4)c3)c3ccc4sc5cc6ccccc6cc5c4c3)ccc2-c2ccc3ccccc3c2)cc1]

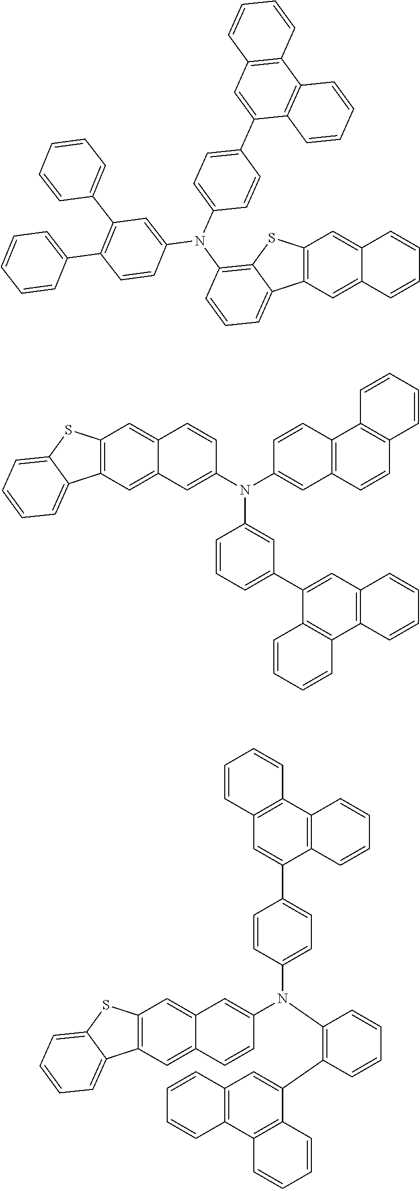 [SMILES: c1ccc(-c2ccc(N(c3ccc(-c4cc5ccccc5c5ccccc45)cc3)c3cccc4c3sc3cc5ccccc5cc34)cc2-c2ccccc2)cc1 ; c1cc(-c2cc3ccccc3c3ccccc23)cc(N(c2ccc3cc4sc5ccccc5c4cc3c2)c2ccc3c(ccc4ccccc43)c2)c1 ; c1ccc(N(c2ccc(-c3cc4ccccc4c4ccccc34)cc2)c2ccc3cc4c(cc3c2)sc2ccccc24)c(-c2cc3ccccc3c3ccccc23)c1]

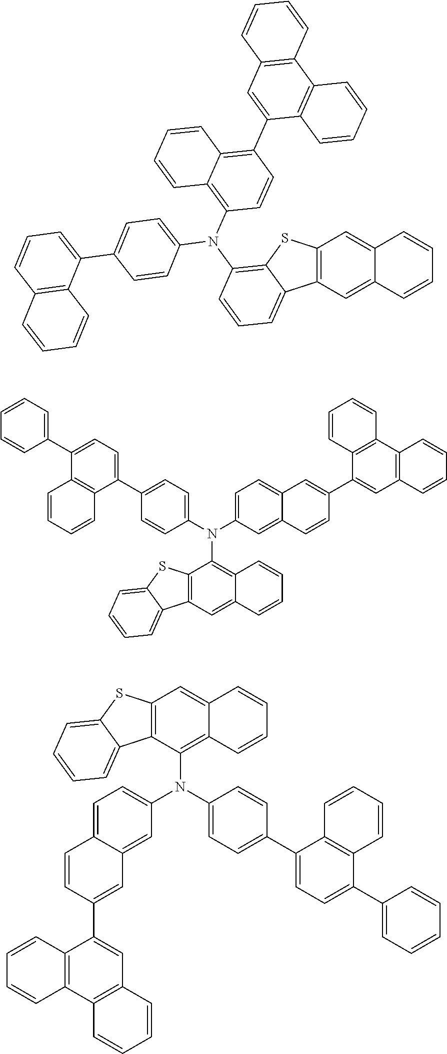 [SMILES: c1ccc2cc3c(cc2c1)sc1c(N(c2ccc(-c4cccc5ccccc45)cc2)c2ccc(-c4cc5ccccc5c5ccccc45)c4ccccc24)cccc13 ; c1ccc(-c2ccc(-c3ccc(N(c4ccc5cc(-c6cc7ccccc7c7ccccc67)ccc5c4)c4c5ccccc5cc5c4sc4ccccc45)cc3)c3ccccc23)cc1 ; c1ccc(-c2ccc(-c3ccc(N(c4ccc5ccc(-c6cc7ccccc7c7ccccc67)cc5c4)c4c5ccccc5cc5sc6ccccc6c45)cc3)c3ccccc23)cc1]

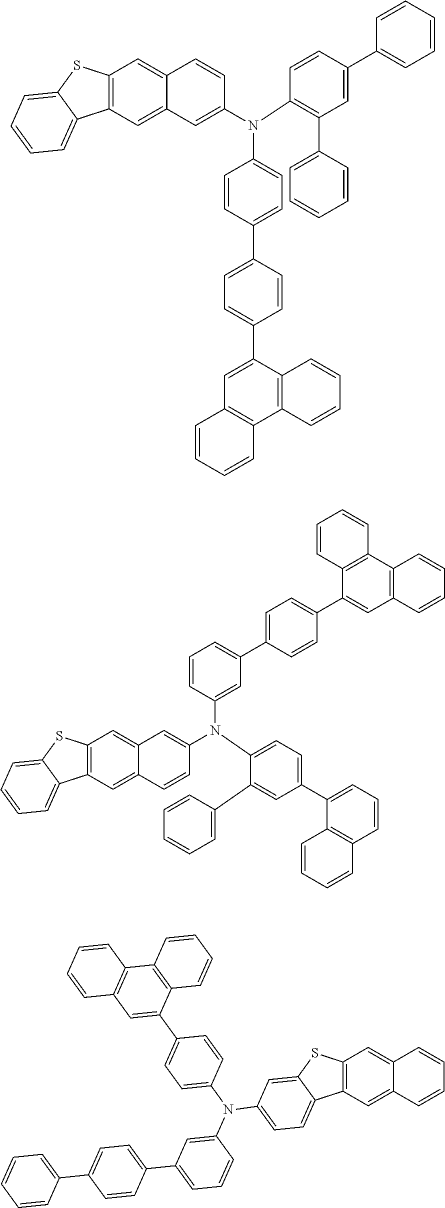 [SMILES: c1ccc(-c2ccc(N(c3ccc(-c4ccc(-c5cc6ccccc6c6ccccc56)cc4)cc3)c3ccc4cc5sc6ccccc6c5cc4c3)c(-c3ccccc3)c2)cc1 ; c1ccc(-c2cc(-c3cccc4ccccc34)ccc2N(c2cccc(-c3ccc(-c4cc5ccccc5c5ccccc45)cc3)c2)c2ccc3cc4c(cc3c2)sc2ccccc24)cc1 ; c1ccc(-c2ccc(-c3cccc(N(c4ccc(-c5cc6ccccc6c6ccccc56)cc4)c4ccc5c(c4)sc4cc6ccccc6cc45)c3)cc2)cc1]

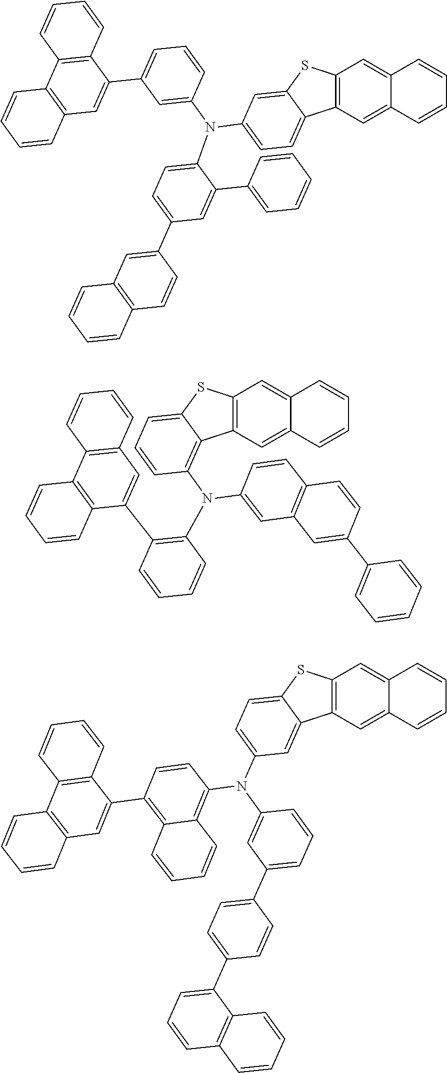

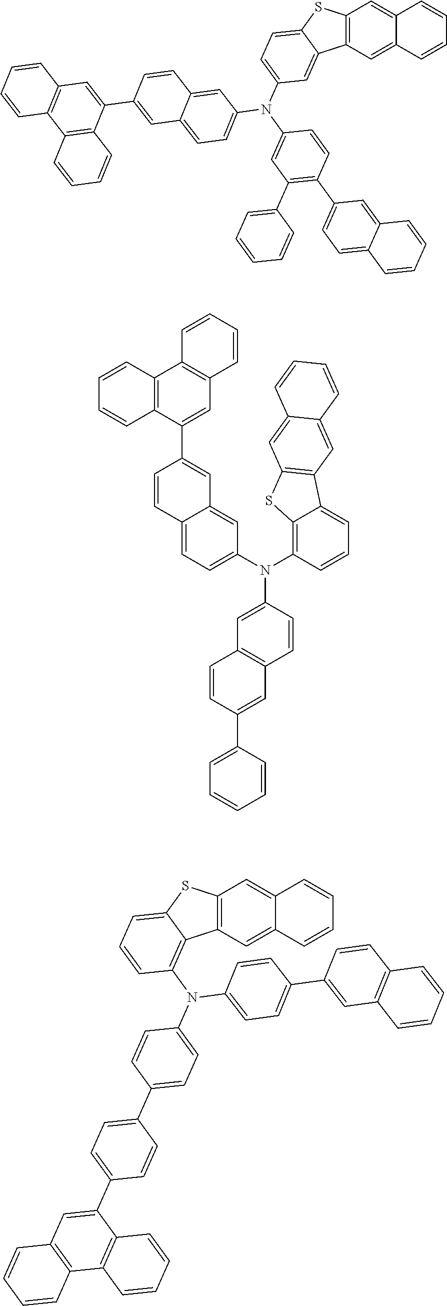 [SMILES: c1ccc(-c2cc(N(c3ccc4cc(-c5cc6ccccc6c6ccccc56)ccc4c3)c3ccc4sc5cc6ccccc6cc5c4c3)ccc2-c2ccc3ccccc3c2)cc1 ; c1ccc(-c2ccc3cc(N(c4ccc5ccc(-c6cc7ccccc7c7ccccc67)cc5c4)c4cccc5c4sc4cc6ccccc6cc45)ccc3c2)cc1 ; c1ccc2cc(-c3ccc(N(c4ccc(-c5ccc(-c6cc7ccccc7c7ccccc67)cc5)cc4)c4cccc5sc6cc7ccccc7cc6c45)cc3)ccc2c1]

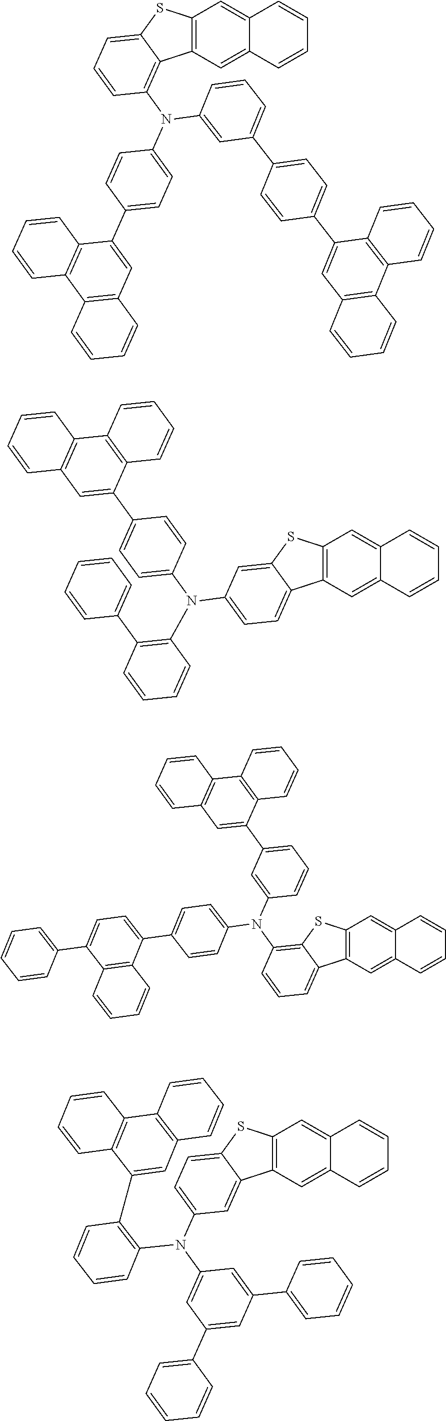 [SMILES: c1cc(-c2ccc(-c3cc4ccccc4c4ccccc34)cc2)cc(N(c2ccc(-c3cc4ccccc4c4ccccc34)cc2)c2cccc3sc4cc5ccccc5cc4c23)c1 ; c1ccc(-c2ccccc2N(c2ccc(-c3cc4ccccc4c4ccccc34)cc2)c2ccc3c(c2)sc2cc4ccccc4cc23)cc1 ; c1ccc(-c2ccc(-c3ccc(N(c4cccc(-c5cc6ccccc6c6ccccc56)c4)c4cccc5c4sc4cc6ccccc6cc45)cc3)c3ccccc23)cc1 ; c1ccc(-c2cc(-c3ccccc3)cc(N(c3ccc4sc5cc6ccccc6cc5c4c3)c3ccccc3-c3cc4ccccc4c4ccccc34)c2)cc1]

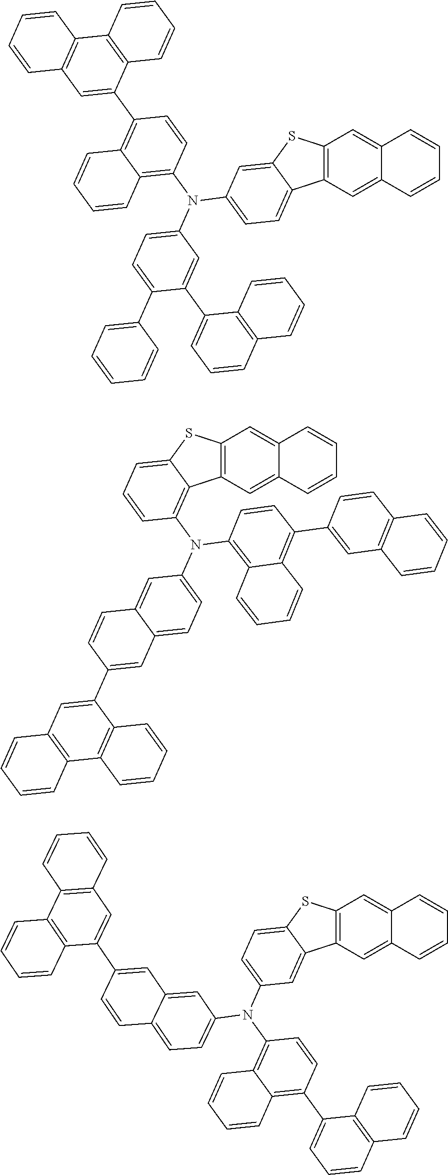

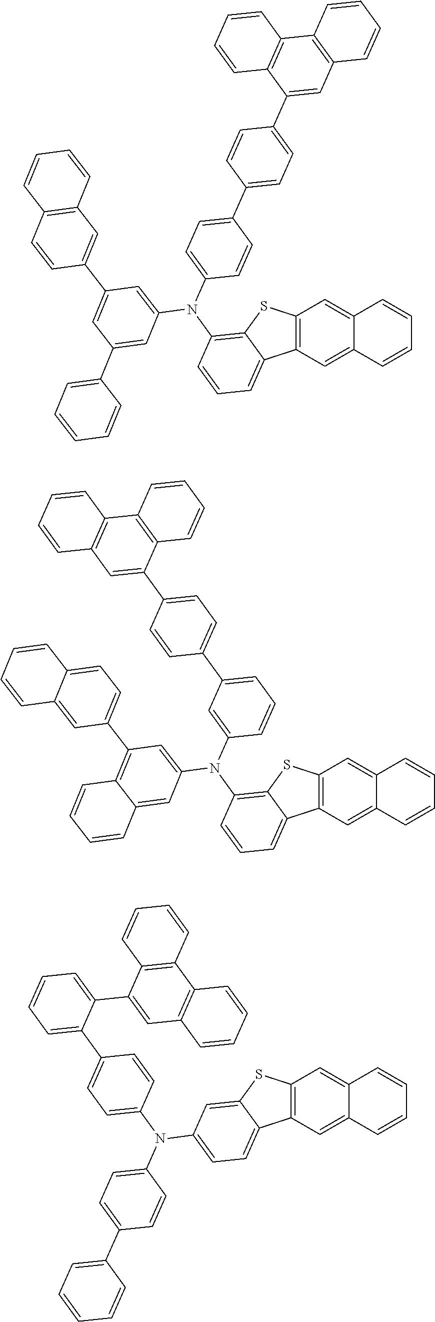 [SMILES: c1ccc(-c2cc(-c3ccc4ccccc4c3)cc(N(c3ccc(-c4ccc(-c5cc6ccccc6c6ccccc56)cc4)cc3)c3cccc4c3sc3cc5ccccc5cc34)c2)cc1 ; c1cc(-c2ccc(-c3cc4ccccc4c4ccccc34)cc2)cc(N(c2cc(-c3ccc4ccccc4c3)c3ccccc3c2)c2cccc3c2sc2cc4ccccc4cc23)c1 ; c1ccc(-c2ccc(N(c3ccc(-c4ccccc4-c4cc5ccccc5c5ccccc45)cc3)c3ccc4c(c3)sc3cc5ccccc5cc34)cc2)cc1]

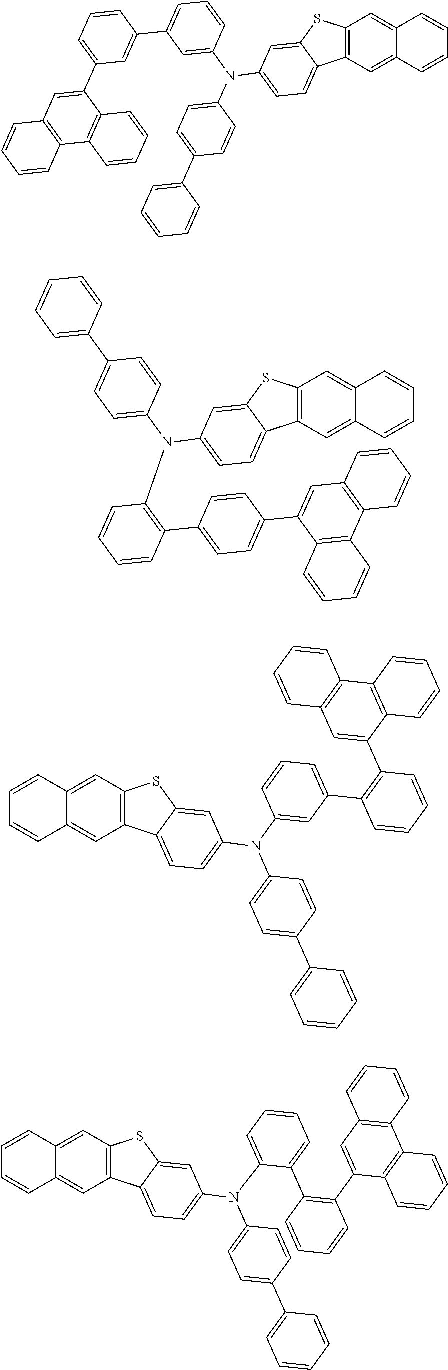 [SMILES: c1ccc(-c2ccc(N(c3cccc(-c4cccc(-c5cc6ccccc6c6ccccc56)c4)c3)c3ccc4c(c3)sc3cc5ccccc5cc34)cc2)cc1 ; c1ccc(-c2ccc(N(c3ccc4c(c3)sc3cc5ccccc5cc34)c3ccccc3-c3ccc(-c4cc5ccccc5c5ccccc45)cc3)cc2)cc1 ; c1ccc(-c2ccc(N(c3cccc(-c4ccccc4-c4cc5ccccc5c5ccccc45)c3)c3ccc4c(c3)sc3cc5ccccc5cc34)cc2)cc1 ; c1ccc(-c2ccc(N(c3ccc4c(c3)sc3cc5ccccc5cc34)c3ccccc3-c3ccccc3-c3cc4ccccc4c4ccccc34)cc2)cc1]

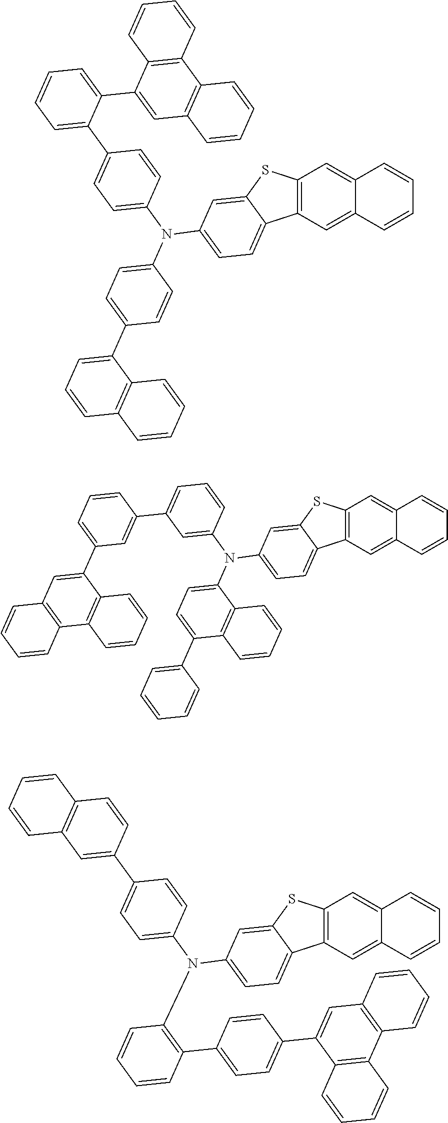 [SMILES: c1ccc(-c2cc3ccccc3c3ccccc23)c(-c2ccc(N(c3ccc(-c4cccc5ccccc45)cc3)c3ccc4c(c3)sc3cc5ccccc5cc34)cc2)c1 ; c1ccc(-c2ccc(N(c3cccc(-c4cccc(-c5cc6ccccc6c6ccccc56)c4)c3)c3ccc4c(c3)sc3cc5ccccc5cc34)c3ccccc23)cc1 ; c1ccc(N(c2ccc(-c3ccc4ccccc4c3)cc2)c2ccc3c(c2)sc2cc4ccccc4cc23)c(-c2ccc(-c3cc4ccccc4c4ccccc34)cc2)c1]

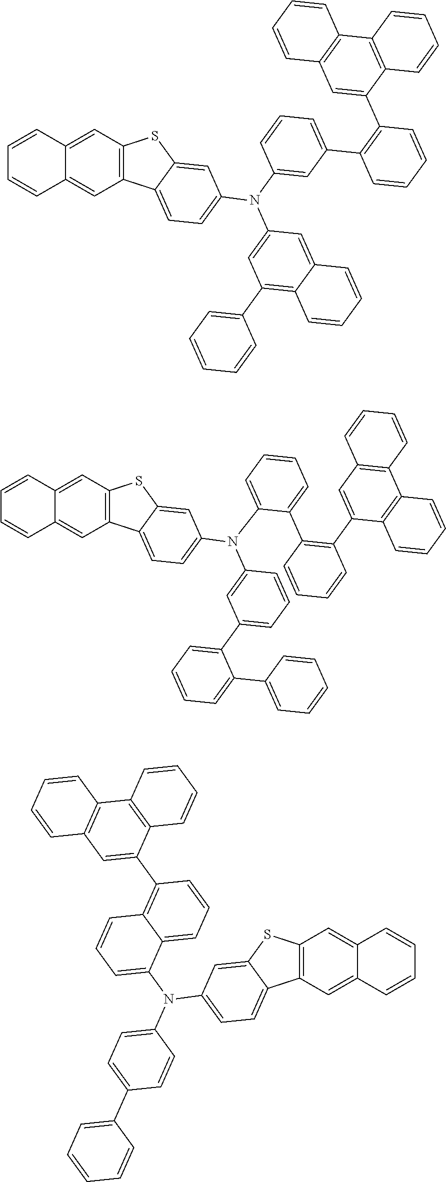 [SMILES: c1ccc(-c2cc(N(c3cccc(-c4ccccc4-c4cc5ccccc5c5ccccc45)c3)c3ccc4c(c3)sc3cc5ccccc5cc34)cc3ccccc23)cc1 ; c1ccc(-c2ccccc2-c2cccc(N(c3ccc4c(c3)sc3cc5ccccc5cc34)c3ccccc3-c3ccccc3-c3cc4ccccc4c4ccccc34)c2)cc1 ; c1ccc(-c2ccc(N(c3ccc4c(c3)sc3cc5ccccc5cc34)c3cccc4c(-c5cc6ccccc6c6ccccc56)cccc34)cc2)cc1]

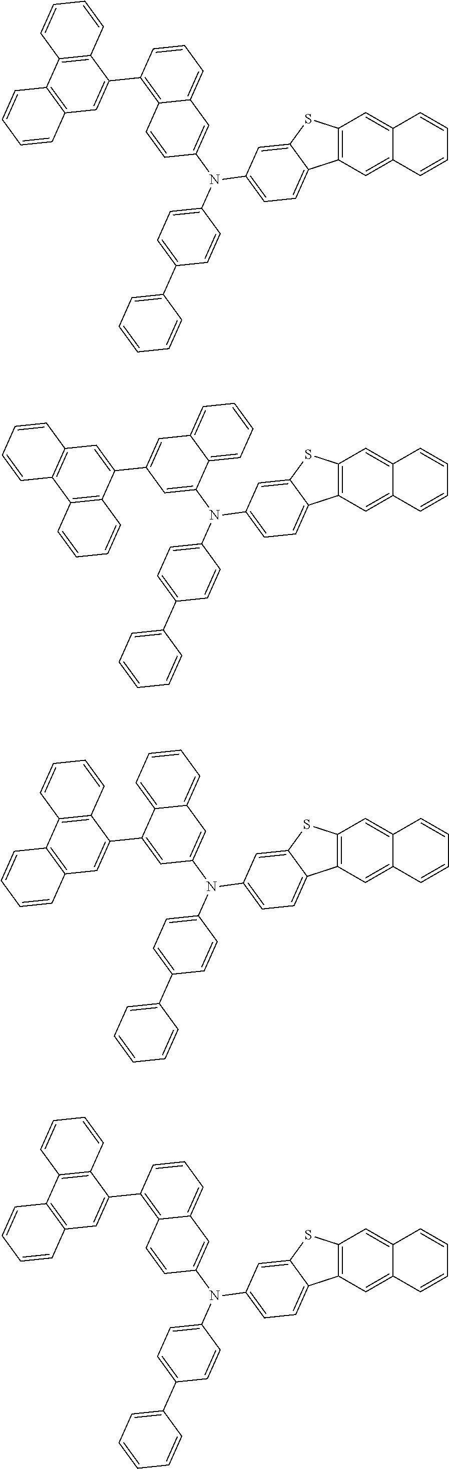 [SMILES: c1ccc(-c2ccc(N(c3ccc4c(-c5cc6ccccc6c6ccccc56)cccc4c3)c3ccc4c(c3)sc3cc5ccccc5cc34)cc2)cc1 ; c1ccc(-c2ccc(N(c3ccc4c(c3)sc3cc5ccccc5cc34)c3cc(-c4cc5ccccc5c5ccccc45)cc4ccccc34)cc2)cc1 ; c1ccc(-c2ccc(N(c3cc(-c4cc5ccccc5c5ccccc45)c4ccccc4c3)c3ccc4c(c3)sc3cc5ccccc5cc34)cc2)cc1 ; c1ccc(-c2ccc(N(c3ccc4c(-c5cc6ccccc6c6ccccc56)cccc4c3)c3ccc4c(c3)sc3cc5ccccc5cc34)cc2)cc1]

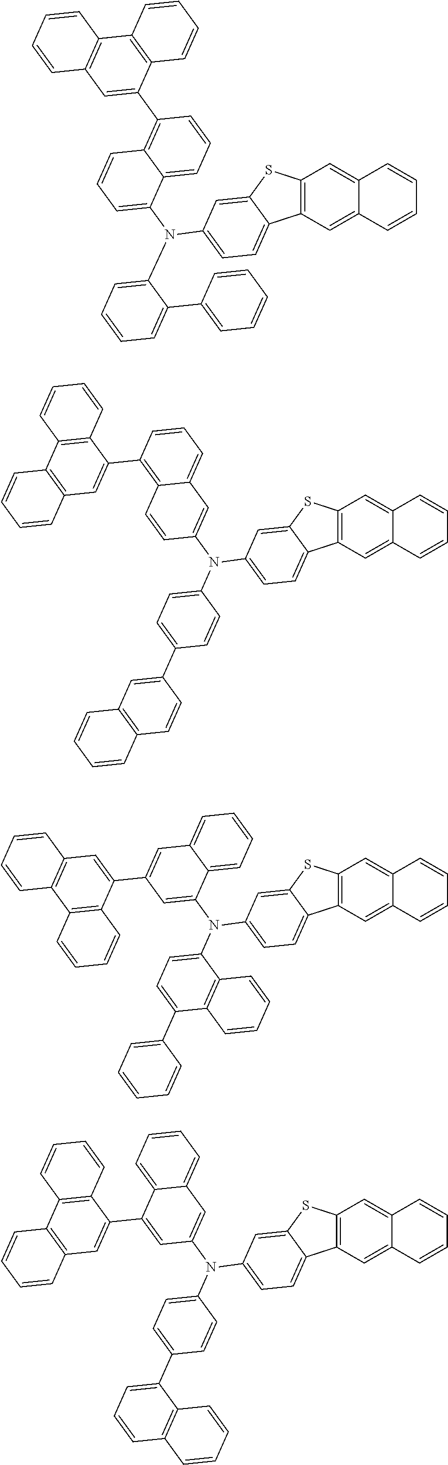

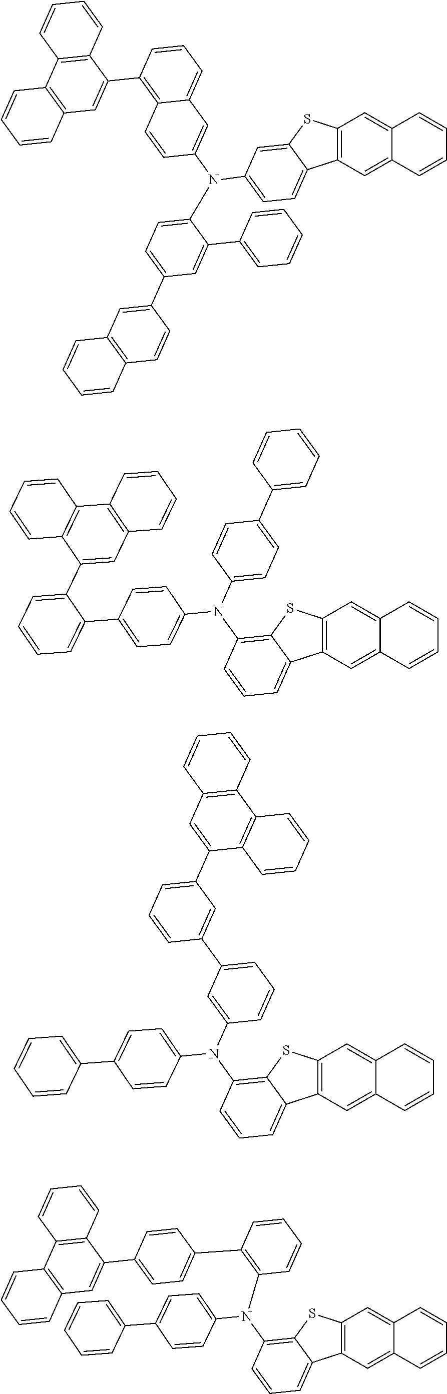

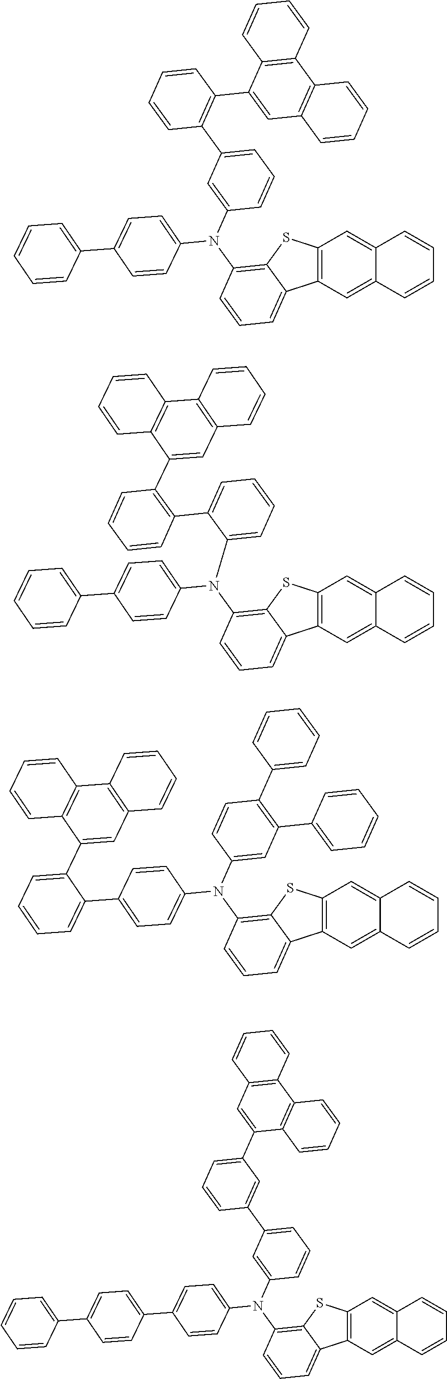

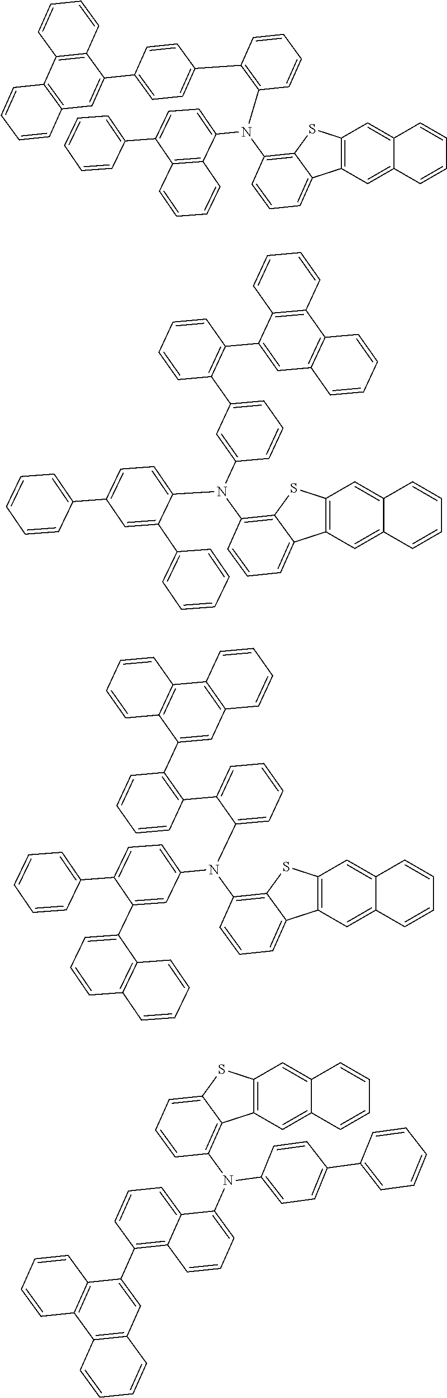

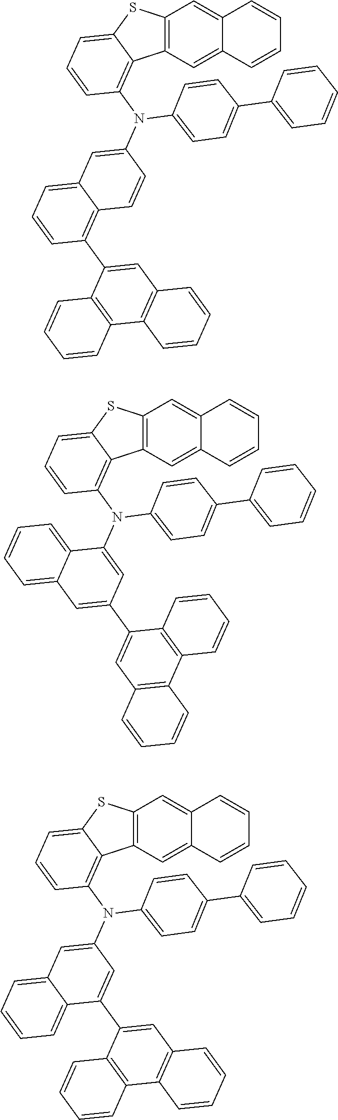 [SMILES: c1ccc(-c2ccc(N(c3ccc4c(-c5cc6ccccc6c6ccccc56)cccc4c3)c3cccc4sc5cc6ccccc6cc5c34)cc2)cc1 ; c1ccc(-c2ccc(N(c3cc(-c4cc5ccccc5c5ccccc45)cc4ccccc34)c3cccc4sc5cc6ccccc6cc5c34)cc2)cc1 ; c1ccc(-c2ccc(N(c3cc(-c4cc5ccccc5c5ccccc45)c4ccccc4c3)c3cccc4sc5cc6ccccc6cc5c34)cc2)cc1]

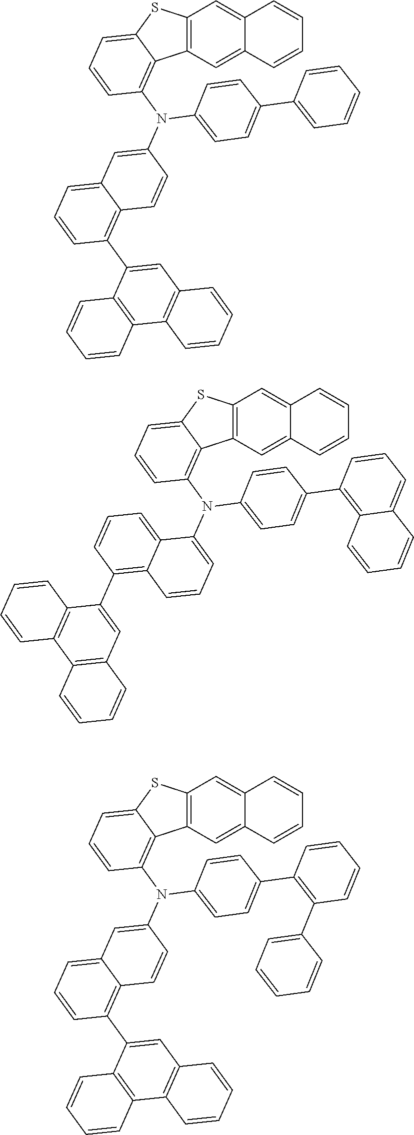 [SMILES: c1ccc(-c2ccc(N(c3ccc4c(-c5cc6ccccc6c6ccccc56)cccc4c3)c3cccc4sc5cc6ccccc6cc5c34)cc2)cc1 ; c1ccc2cc3c(cc2c1)sc1cccc(N(c2ccc(-c4cccc5ccccc45)cc2)c2cccc4c(-c5cc6ccccc6c6ccccc56)cccc24)c13 ; c1ccc(-c2ccccc2-c2ccc(N(c3ccc4c(-c5cc6ccccc6c6ccccc56)cccc4c3)c3cccc4sc5cc6ccccc6cc5c34)cc2)cc1]

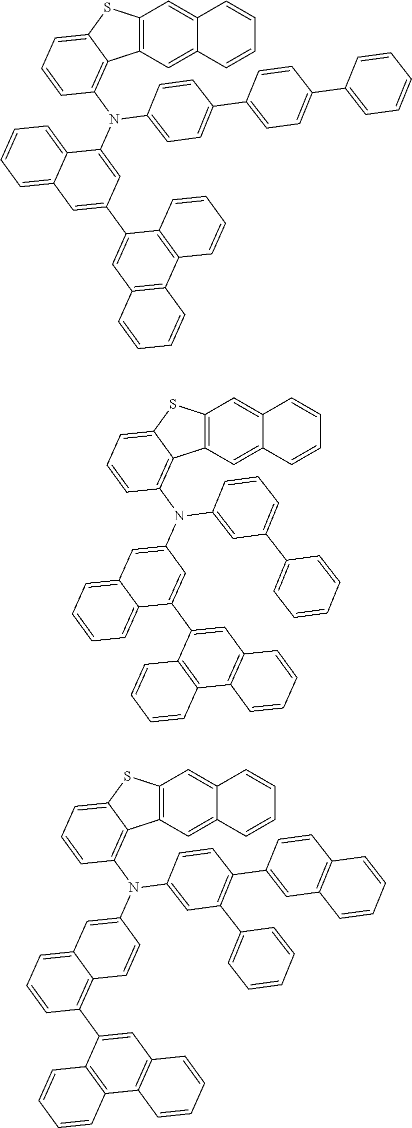 [SMILES: c1ccc(-c2ccc(-c3ccc(N(c4cc(-c5cc6ccccc6c6ccccc56)cc5ccccc45)c4cccc5sc6cc7ccccc7cc6c45)cc3)cc2)cc1 ; c1ccc(-c2cccc(N(c3cc(-c4cc5ccccc5c5ccccc45)c4ccccc4c3)c3cccc4sc5cc6ccccc6cc5c34)c2)cc1 ; c1ccc(-c2cc(N(c3ccc4c(-c5cc6ccccc6c6ccccc56)cccc4c3)c3cccc4sc5cc6ccccc6cc5c34)ccc2-c2ccc3ccccc3c2)cc1]

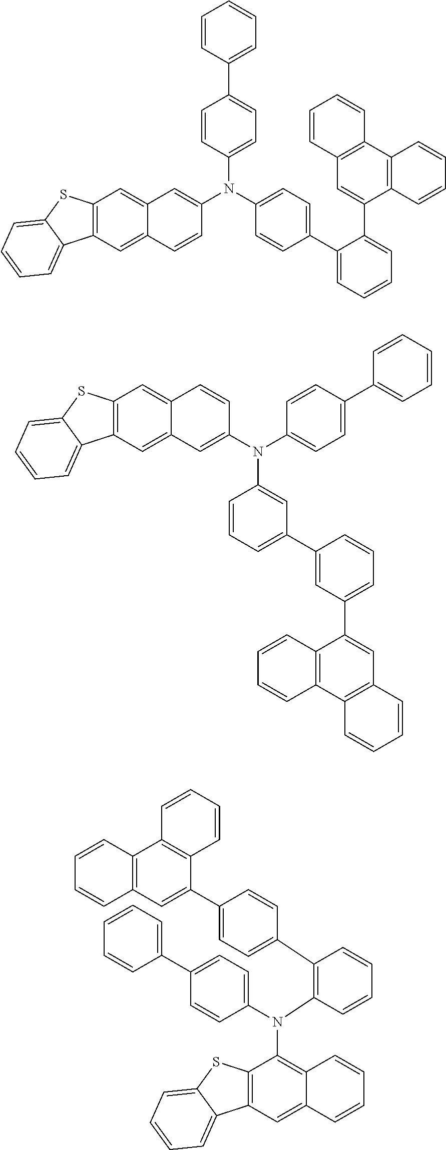 [SMILES: c1ccc(-c2ccc(N(c3ccc(-c4ccccc4-c4cc5ccccc5c5ccccc45)cc3)c3ccc4cc5c(cc4c3)sc3ccccc35)cc2)cc1 ; c1ccc(-c2ccc(N(c3cccc(-c4cccc(-c5cc6ccccc6c6ccccc56)c4)c3)c3ccc4cc5sc6ccccc6c5cc4c3)cc2)cc1 ; c1ccc(-c2ccc(N(c3ccccc3-c3ccc(-c4cc5ccccc5c5ccccc45)cc3)c3c4ccccc4cc4c3sc3ccccc34)cc2)cc1]

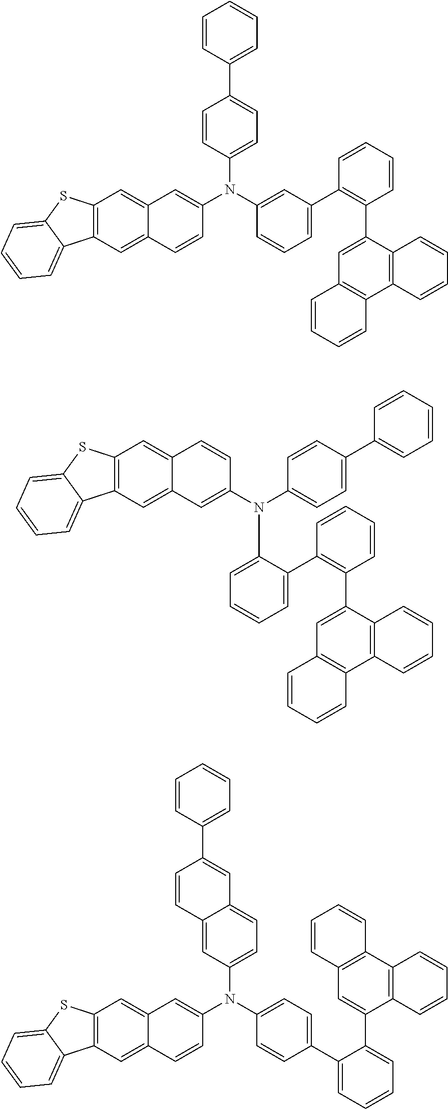 [SMILES: c1ccc(-c2ccc(N(c3cccc(-c4ccccc4-c4cc5ccccc5c5ccccc45)c3)c3ccc4cc5c(cc4c3)sc3ccccc35)cc2)cc1 ; c1ccc(-c2ccc(N(c3ccc4cc5sc6ccccc6c5cc4c3)c3ccccc3-c3ccccc3-c3cc4ccccc4c4ccccc34)cc2)cc1 ; c1ccc(-c2ccc3cc(N(c4ccc(-c5ccccc5-c5cc6ccccc6c6ccccc56)cc4)c4ccc5cc6c(cc5c4)sc4ccccc46)ccc3c2)cc1]

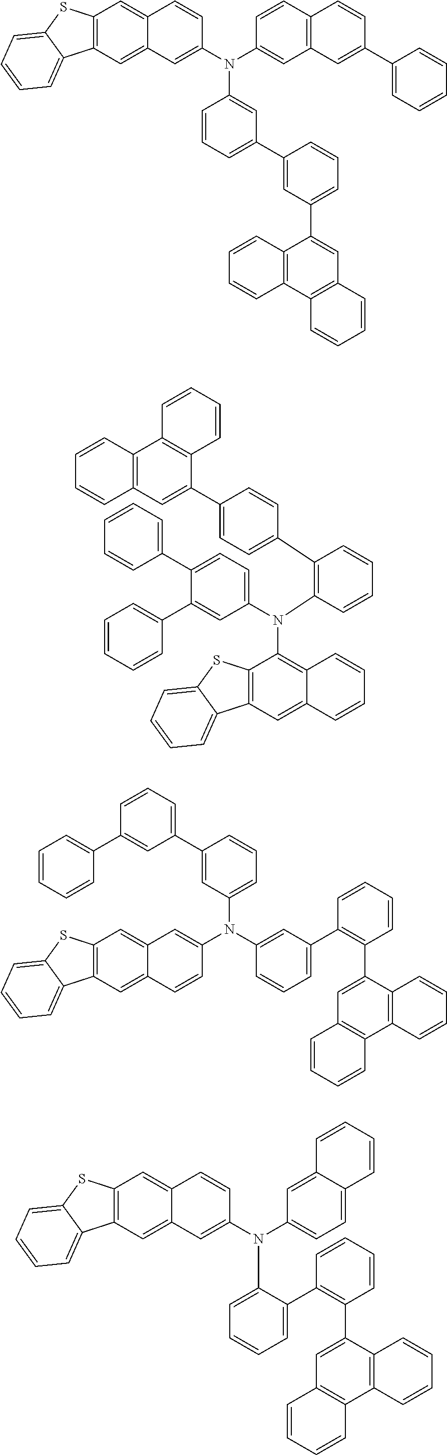

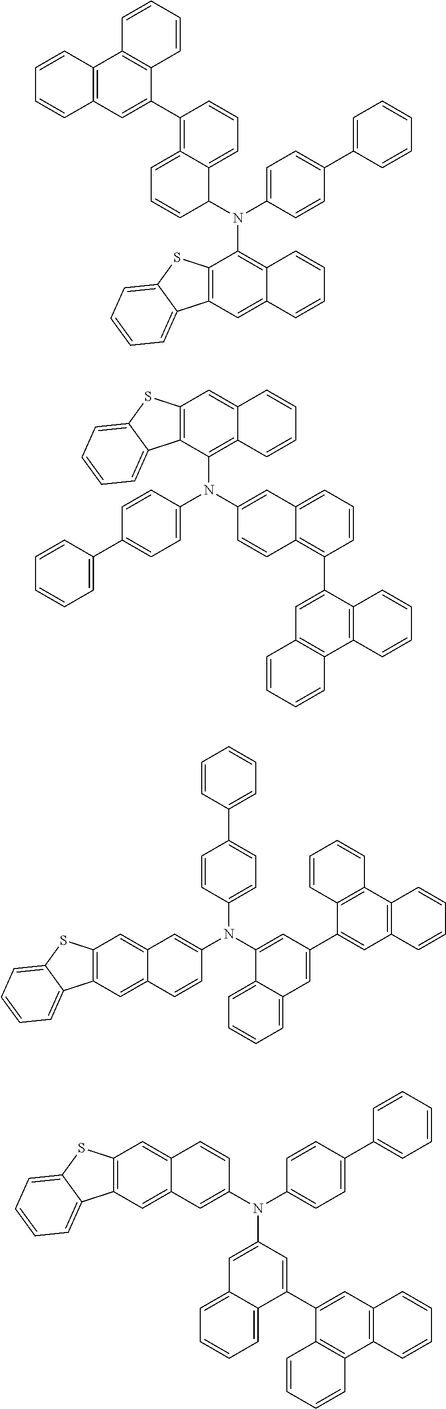

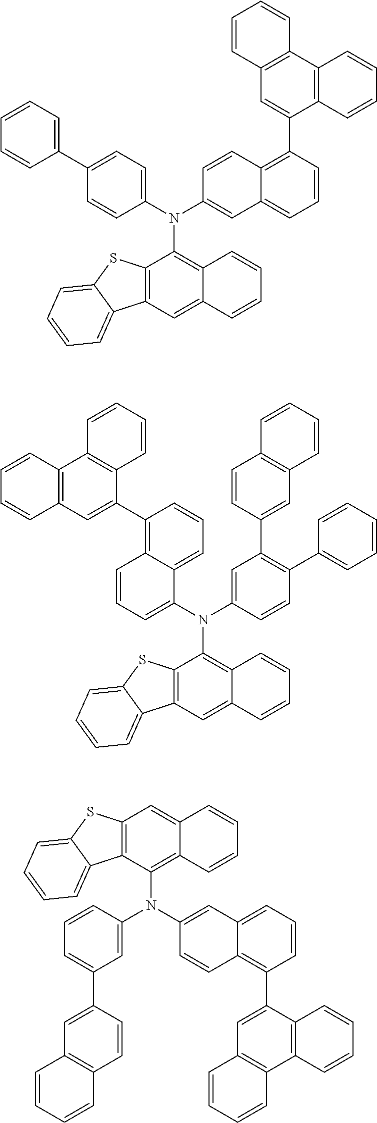 [SMILES: c1ccc(-c2ccc(N(c3ccc4c(-c5cc6ccccc6c6ccccc56)cccc4c3)c3c4ccccc4cc4c3sc3ccccc34)cc2)cc1 ; c1ccc(-c2ccc(N(c3cccc4c(-c5cc6ccccc6c6ccccc56)cccc34)c3c4ccccc4cc4c3sc3ccccc34)cc2-c2ccc3ccccc3c2)cc1 ; c1cc(-c2ccc3ccccc3c2)cc(N(c2ccc3c(-c4cc5ccccc5c5ccccc45)cccc3c2)c2c3ccccc3cc3sc4ccccc4c23)c1]

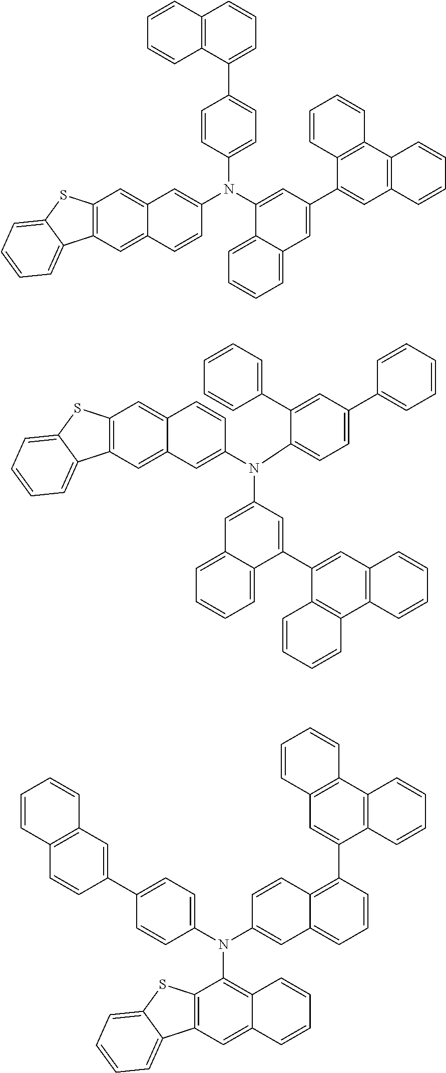 [SMILES: c1ccc2c(-c3ccc(N(c4ccc5cc6c(cc5c4)sc4ccccc46)c4cc(-c5cc6ccccc6c6ccccc56)cc5ccccc45)cc3)cccc2c1 ; c1ccc(-c2ccc(N(c3ccc4cc5sc6ccccc6c5cc4c3)c3cc(-c4cc5ccccc5c5ccccc45)c4ccccc4c3)c(-c3ccccc3)c2)cc1 ; c1ccc2cc(-c3ccc(N(c4ccc5c(-c6cc7ccccc7c7ccccc67)cccc5c4)c4c5ccccc5cc5c4sc4ccccc45)cc3)ccc2c1]

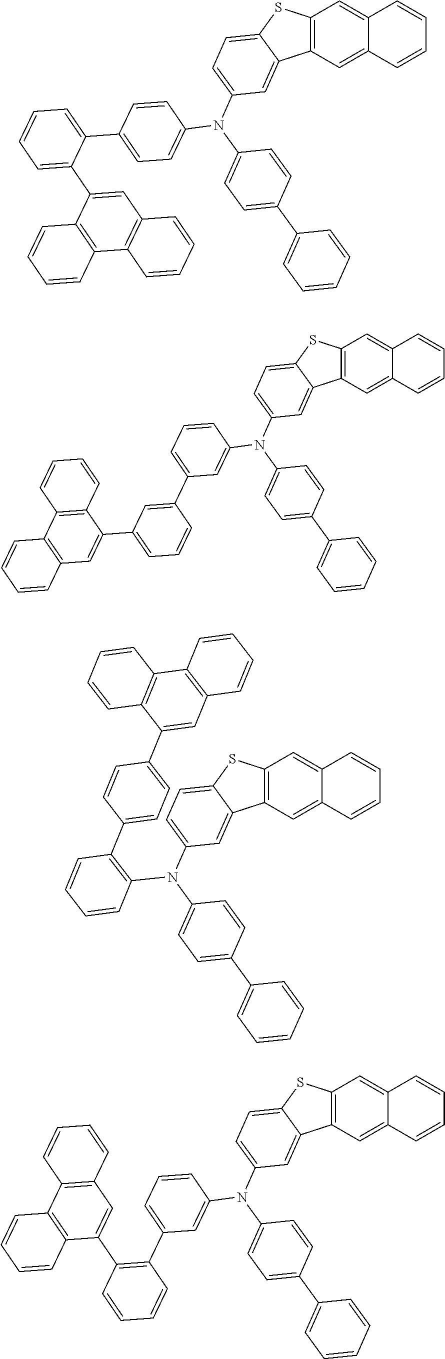

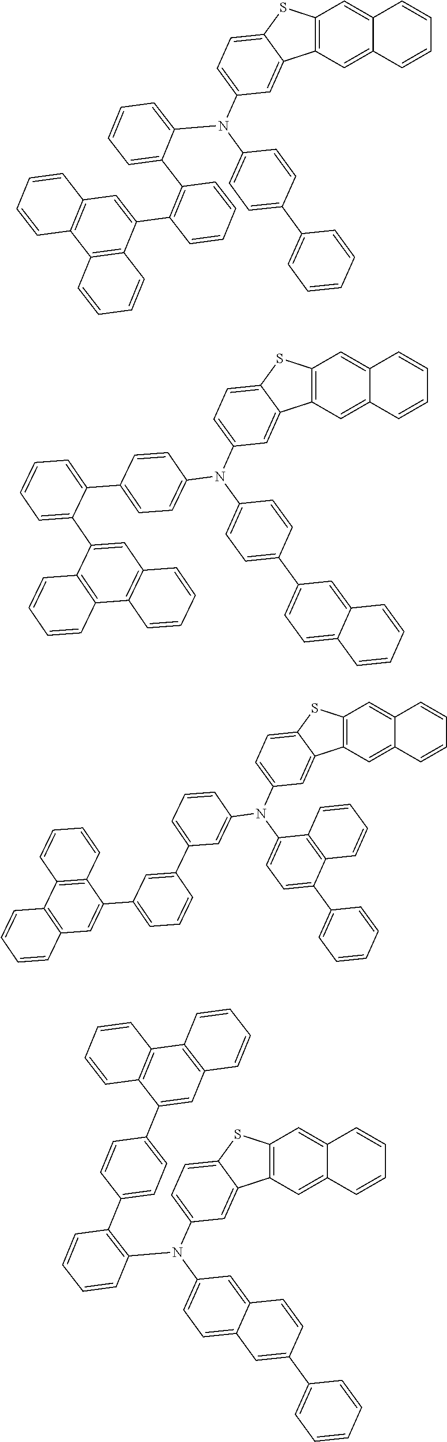

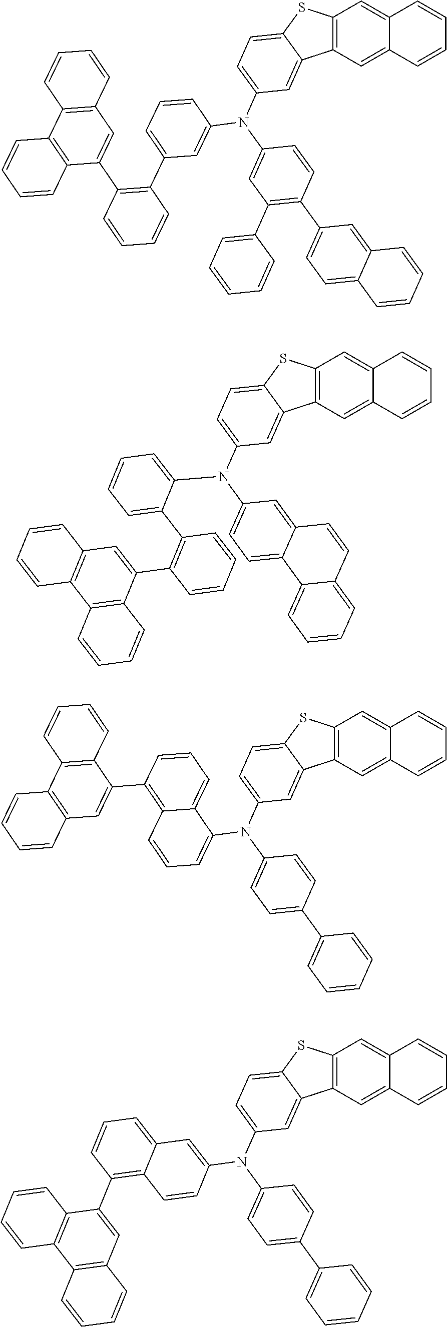

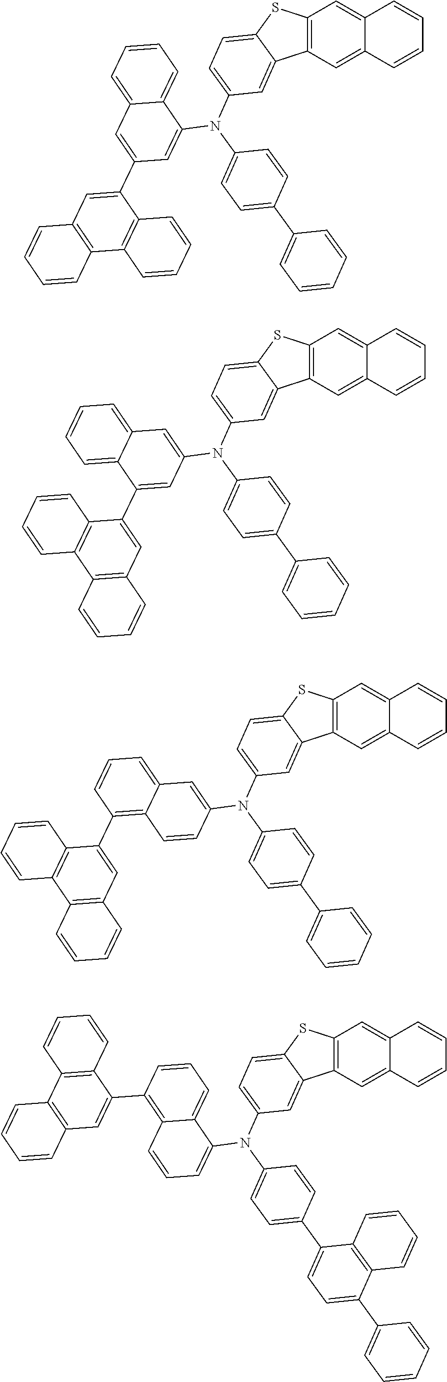

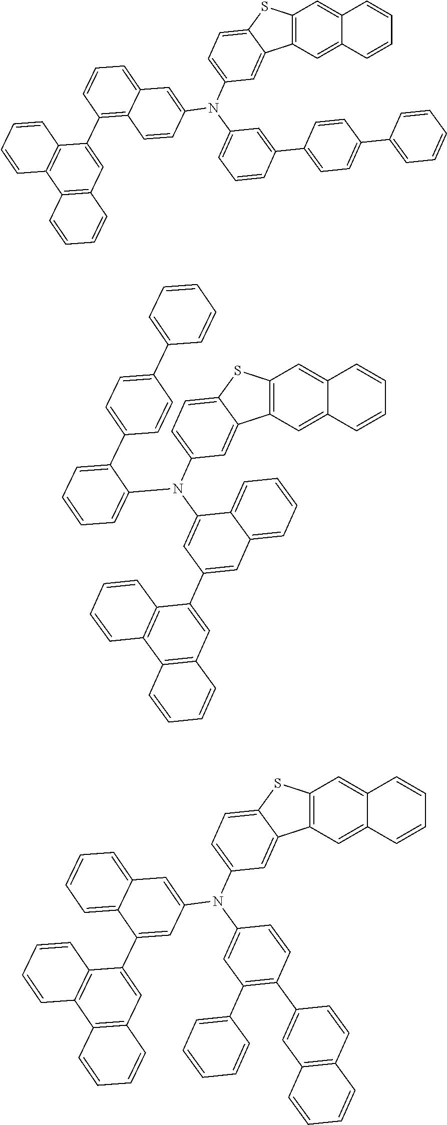 [SMILES: c1ccc(-c2ccc(-c3cccc(N(c4ccc5c(-c6cc7ccccc7c7ccccc67)cccc5c4)c4ccc5sc6cc7ccccc7cc6c5c4)c3)cc2)cc1 ; c1ccc(-c2ccc(-c3ccccc3N(c3ccc4sc5cc6ccccc6cc5c4c3)c3cc(-c4cc5ccccc5c5ccccc45)cc4ccccc34)cc2)cc1 ; c1ccc(-c2cc(N(c3cc(-c4cc5ccccc5c5ccccc45)c4ccccc4c3)c3ccc4sc5cc6ccccc6cc5c4c3)ccc2-c2ccc3ccccc3c2)cc1]

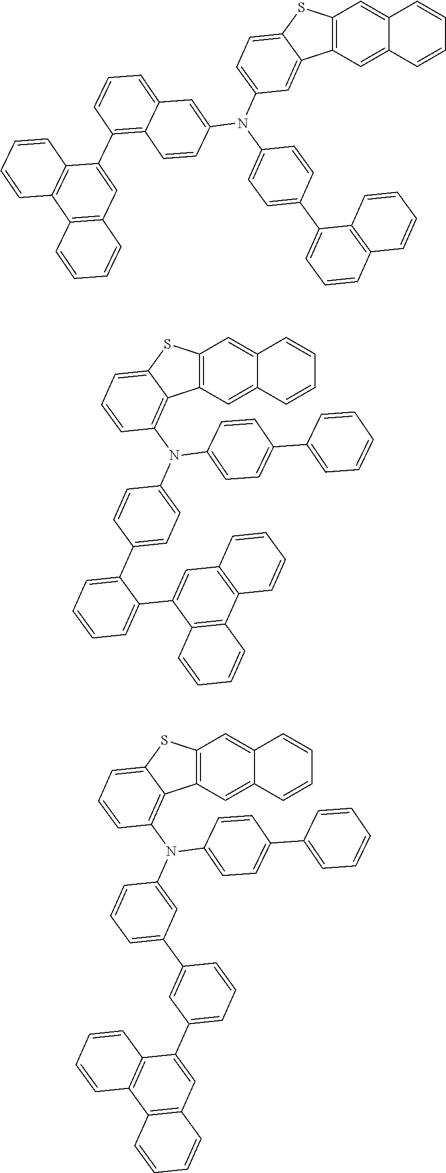 [SMILES: c1ccc2cc3c(cc2c1)sc1ccc(N(c2ccc(-c4cccc5ccccc45)cc2)c2ccc4c(-c5cc6ccccc6c6ccccc56)cccc4c2)cc13 ; c1ccc(-c2ccc(N(c3ccc(-c4ccccc4-c4cc5ccccc5c5ccccc45)cc3)c3cccc4sc5cc6ccccc6cc5c34)cc2)cc1 ; c1ccc(-c2ccc(N(c3cccc(-c4cccc(-c5cc6ccccc6c6ccccc56)c4)c3)c3cccc4sc5cc6ccccc6cc5c34)cc2)cc1]

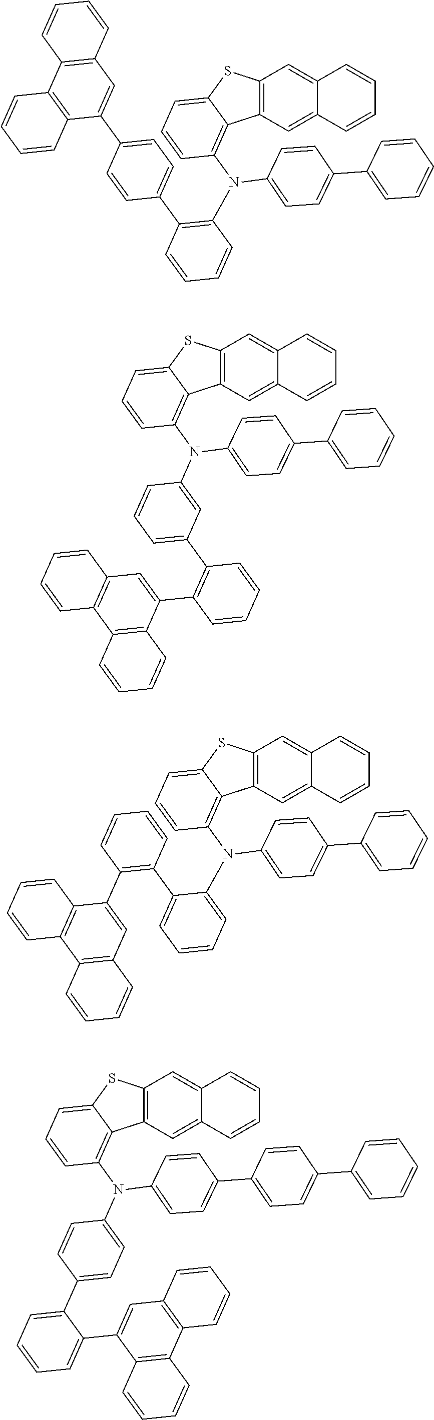 [SMILES: c1ccc(-c2ccc(N(c3ccccc3-c3ccc(-c4cc5ccccc5c5ccccc45)cc3)c3cccc4sc5cc6ccccc6cc5c34)cc2)cc1 ; c1ccc(-c2ccc(N(c3cccc(-c4ccccc4-c4cc5ccccc5c5ccccc45)c3)c3cccc4sc5cc6ccccc6cc5c34)cc2)cc1 ; c1ccc(-c2ccc(N(c3ccccc3-c3ccccc3-c3cc4ccccc4c4ccccc34)c3cccc4sc5cc6ccccc6cc5c34)cc2)cc1 ; c1ccc(-c2ccc(-c3ccc(N(c4ccc(-c5ccccc5-c5cc6ccccc6c6ccccc56)cc4)c4cccc5sc6cc7ccccc7cc6c45)cc3)cc2)cc1]

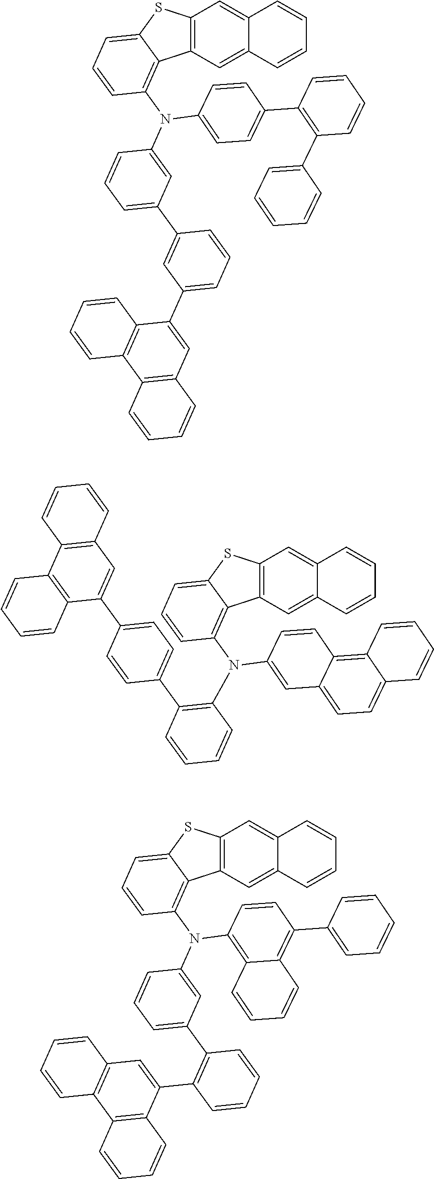

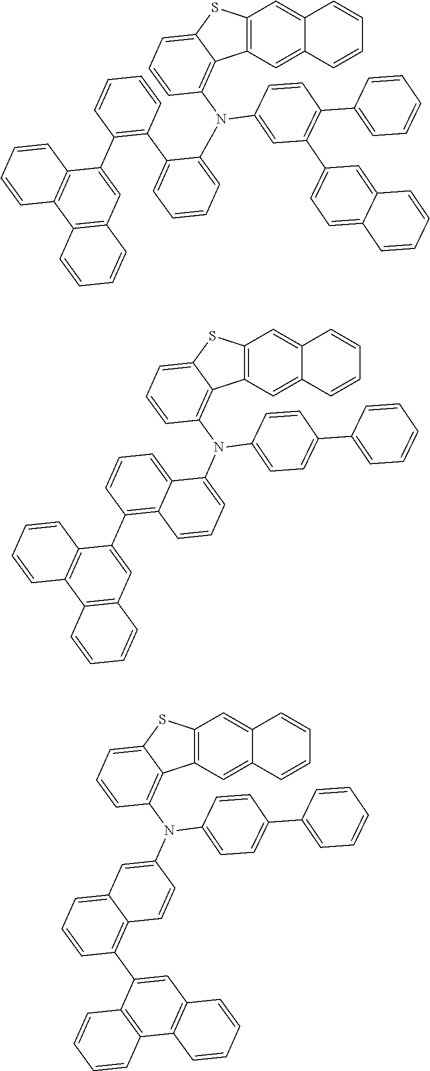 [SMILES: c1ccc(-c2ccc(N(c3ccccc3-c3ccccc3-c3cc4ccccc4c4ccccc34)c3cccc4sc5cc6ccccc6cc5c34)cc2-c2ccc3ccccc3c2)cc1 ; c1ccc(-c2ccc(N(c3cccc4c(-c5cc6ccccc6c6ccccc56)cccc34)c3cccc4sc5cc6ccccc6cc5c34)cc2)cc1 ; c1ccc(-c2ccc(N(c3ccc4c(-c5cc6ccccc6c6ccccc56)cccc4c3)c3cccc4sc5cc6ccccc6cc5c34)cc2)cc1]

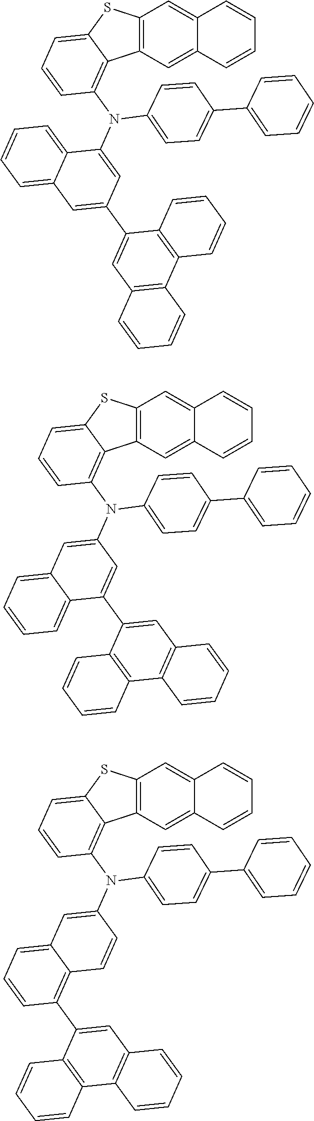 [SMILES: c1ccc(-c2ccc(N(c3cc(-c4cc5ccccc5c5ccccc45)cc4ccccc34)c3cccc4sc5cc6ccccc6cc5c34)cc2)cc1 ; c1ccc(-c2ccc(N(c3cc(-c4cc5ccccc5c5ccccc45)c4ccccc4c3)c3cccc4sc5cc6ccccc6cc5c34)cc2)cc1 ; c1ccc(-c2ccc(N(c3ccc4c(-c5cc6ccccc6c6ccccc56)cccc4c3)c3cccc4sc5cc6ccccc6cc5c34)cc2)cc1]

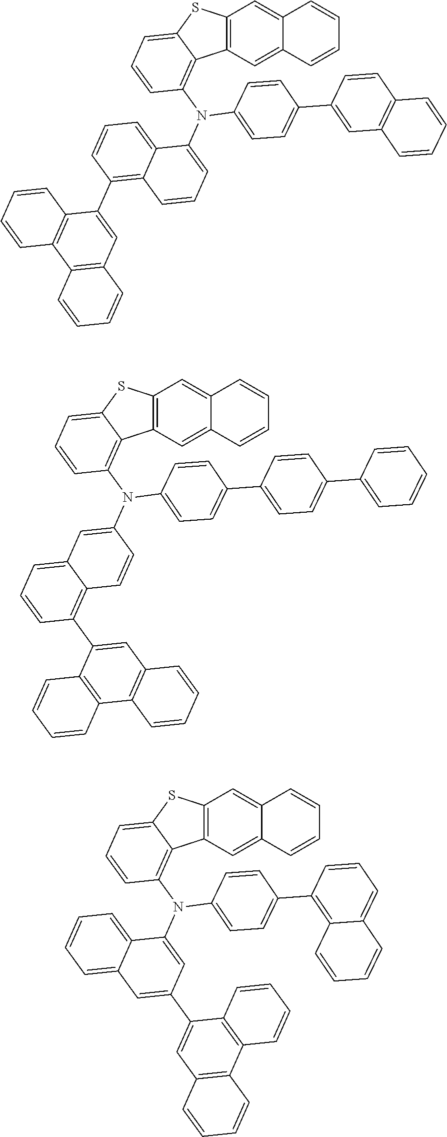 [SMILES: c1ccc2cc(-c3ccc(N(c4cccc5c(-c6cc7ccccc7c7ccccc67)cccc45)c4cccc5sc6cc7ccccc7cc6c45)cc3)ccc2c1 ; c1ccc(-c2ccc(-c3ccc(N(c4ccc5c(-c6cc7ccccc7c7ccccc67)cccc5c4)c4cccc5sc6cc7ccccc7cc6c45)cc3)cc2)cc1 ; c1ccc2cc3c(cc2c1)sc1cccc(N(c2ccc(-c4cccc5ccccc45)cc2)c2cc(-c4cc5ccccc5c5ccccc45)cc4ccccc24)c13]

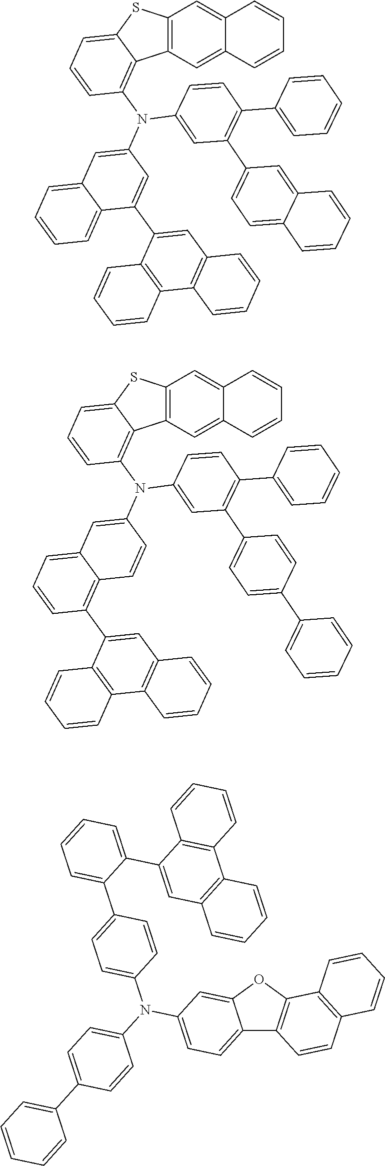 [SMILES: c1ccc(-c2ccc(N(c3cc(-c4cc5ccccc5c5ccccc45)c4ccccc4c3)c3cccc4sc5cc6ccccc6cc5c34)cc2-c2ccc3ccccc3c2)cc1 ; c1ccc(-c2ccc(-c3cc(N(c4ccc5c(-c6cc7ccccc7c7ccccc67)cccc5c4)c4cccc5sc6cc7ccccc7cc6c45)ccc3-c3ccccc3)cc2)cc1 ; c1ccc(-c2ccc(N(c3ccc(-c4ccccc4-c4cc5ccccc5c5ccccc45)cc3)c3ccc4c(c3)oc3c5ccccc5ccc43)cc2)cc1]

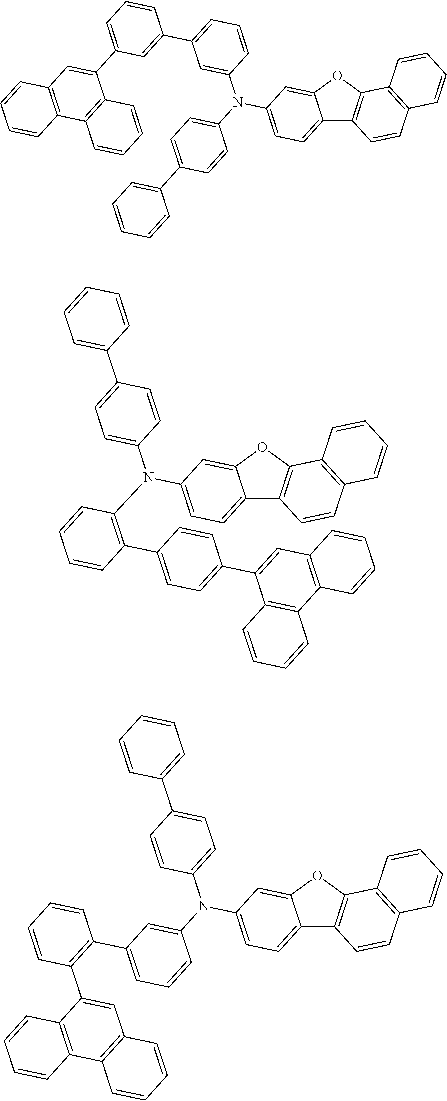 [SMILES: c1ccc(-c2ccc(N(c3cccc(-c4cccc(-c5cc6ccccc6c6ccccc56)c4)c3)c3ccc4c(c3)oc3c5ccccc5ccc43)cc2)cc1 ; c1ccc(-c2ccc(N(c3ccc4c(c3)oc3c5ccccc5ccc43)c3ccccc3-c3ccc(-c4cc5ccccc5c5ccccc45)cc3)cc2)cc1 ; c1ccc(-c2ccc(N(c3cccc(-c4ccccc4-c4cc5ccccc5c5ccccc45)c3)c3ccc4c(c3)oc3c5ccccc5ccc43)cc2)cc1]

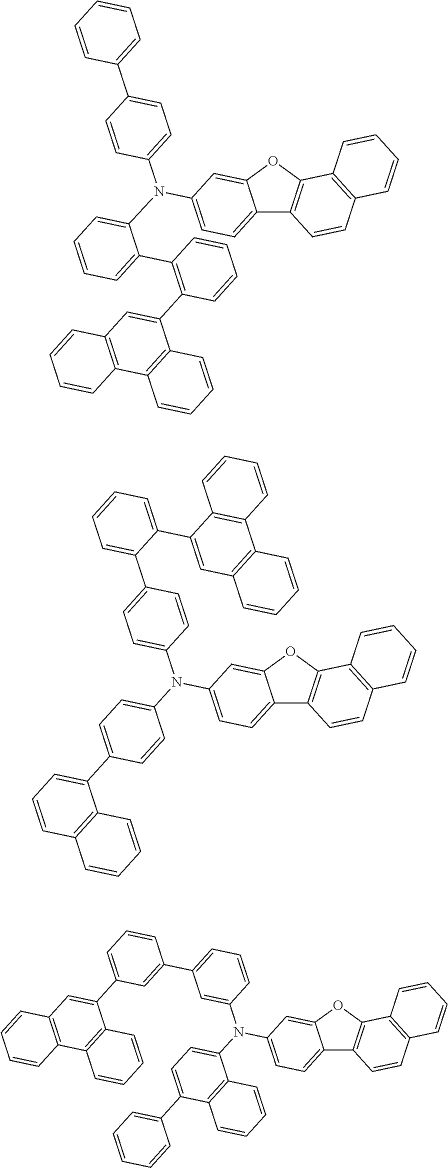 [SMILES: c1ccc(-c2ccc(N(c3ccc4c(c3)oc3c5ccccc5ccc43)c3ccccc3-c3ccccc3-c3cc4ccccc4c4ccccc34)cc2)cc1 ; c1ccc(-c2cc3ccccc3c3ccccc23)c(-c2ccc(N(c3ccc(-c4cccc5ccccc45)cc3)c3ccc4c(c3)oc3c5ccccc5ccc43)cc2)c1 ; c1ccc(-c2ccc(N(c3cccc(-c4cccc(-c5cc6ccccc6c6ccccc56)c4)c3)c3ccc4c(c3)oc3c5ccccc5ccc43)c3ccccc23)cc1]

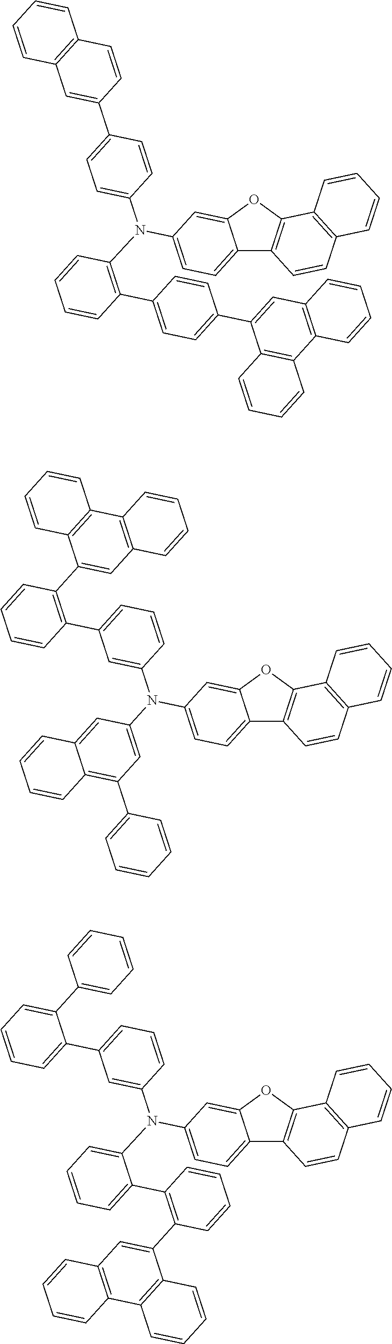 [SMILES: c1ccc(N(c2ccc(-c3ccc4ccccc4c3)cc2)c2ccc3c(c2)oc2c4ccccc4ccc32)c(-c2ccc(-c3cc4ccccc4c4ccccc34)cc2)c1 ; c1ccc(-c2cc(N(c3cccc(-c4ccccc4-c4cc5ccccc5c5ccccc45)c3)c3ccc4c(c3)oc3c5ccccc5ccc43)cc3ccccc23)cc1 ; c1ccc(-c2ccccc2-c2cccc(N(c3ccc4c(c3)oc3c5ccccc5ccc43)c3ccccc3-c3ccccc3-c3cc4ccccc4c4ccccc34)c2)cc1]

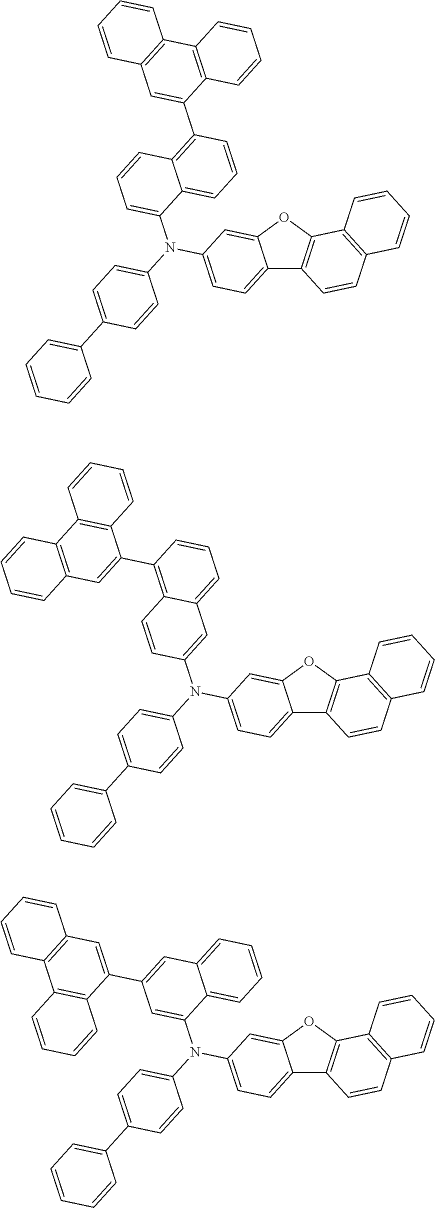 [SMILES: c1ccc(-c2ccc(N(c3ccc4c(c3)oc3c5ccccc5ccc43)c3cccc4c(-c5cc6ccccc6c6ccccc56)cccc34)cc2)cc1 ; c1ccc(-c2ccc(N(c3ccc4c(-c5cc6ccccc6c6ccccc56)cccc4c3)c3ccc4c(c3)oc3c5ccccc5ccc43)cc2)cc1 ; c1ccc(-c2ccc(N(c3ccc4c(c3)oc3c5ccccc5ccc43)c3cc(-c4cc5ccccc5c5ccccc45)cc4ccccc34)cc2)cc1]

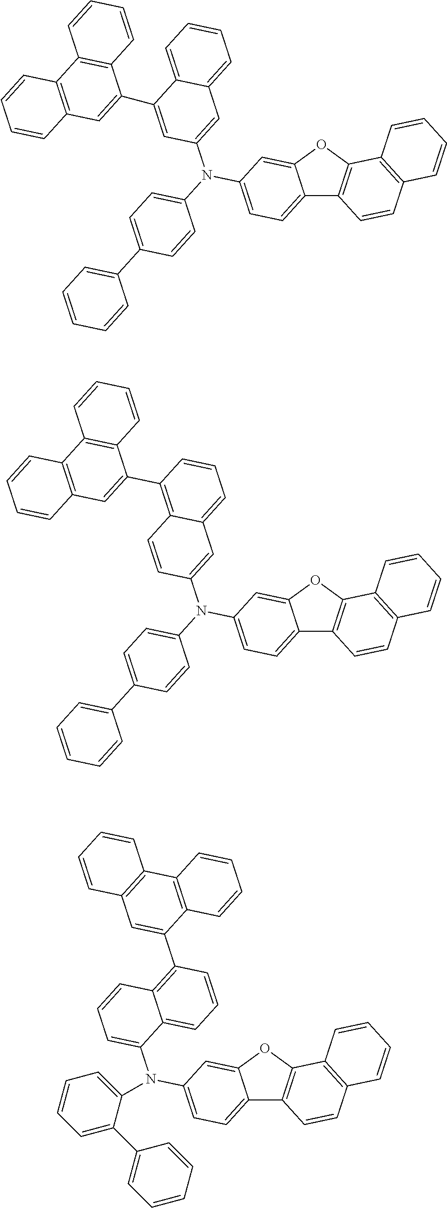 [SMILES: c1ccc(-c2ccc(N(c3cc(-c4cc5ccccc5c5ccccc45)c4ccccc4c3)c3ccc4c(c3)oc3c5ccccc5ccc43)cc2)cc1 ; c1ccc(-c2ccc(N(c3ccc4c(-c5cc6ccccc6c6ccccc56)cccc4c3)c3ccc4c(c3)oc3c5ccccc5ccc43)cc2)cc1 ; c1ccc(-c2ccccc2N(c2ccc3c(c2)oc2c4ccccc4ccc32)c2cccc3c(-c4cc5ccccc5c5ccccc45)cccc23)cc1]

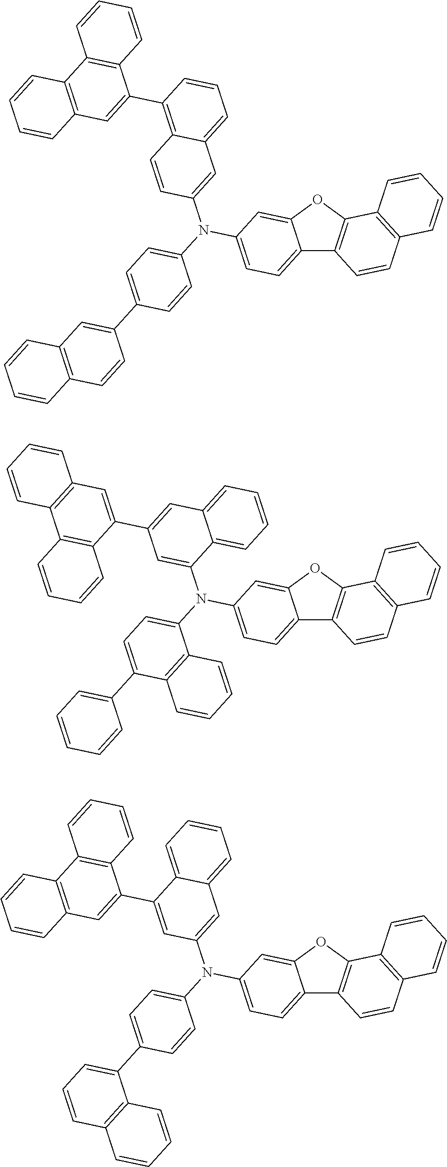

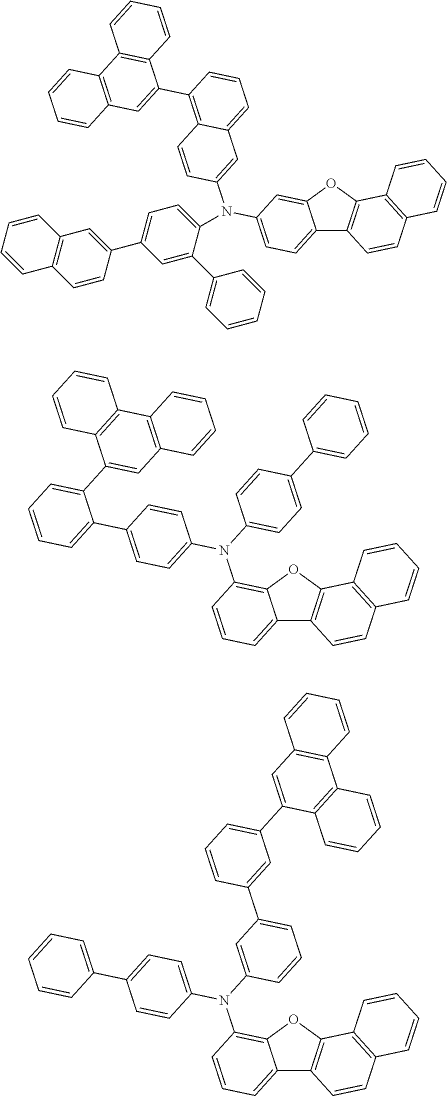 [SMILES: c1ccc(-c2cc(-c3ccc4ccccc4c3)ccc2N(c2ccc3c(-c4cc5ccccc5c5ccccc45)cccc3c2)c2ccc3c(c2)oc2c4ccccc4ccc32)cc1 ; c1ccc(-c2ccc(N(c3ccc(-c4ccccc4-c4cc5ccccc5c5ccccc45)cc3)c3cccc4c3oc3c5ccccc5ccc43)cc2)cc1 ; c1ccc(-c2ccc(N(c3cccc(-c4cccc(-c5cc6ccccc6c6ccccc56)c4)c3)c3cccc4c3oc3c5ccccc5ccc43)cc2)cc1]

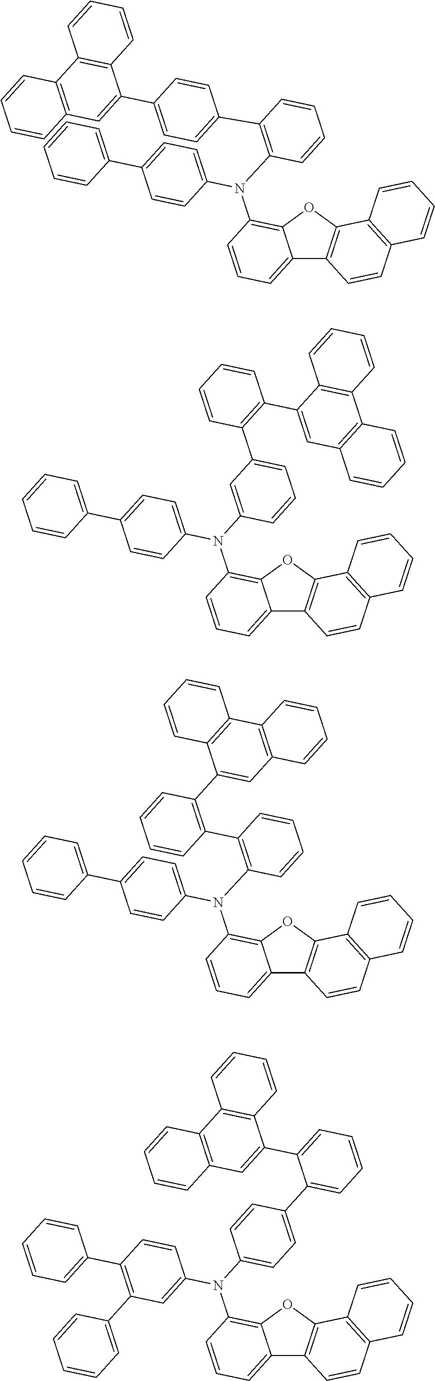 [SMILES: c1ccc(-c2ccc(N(c3ccccc3-c3ccc(-c4cc5ccccc5c5ccccc45)cc3)c3cccc4c3oc3c5ccccc5ccc43)cc2)cc1 ; c1ccc(-c2ccc(N(c3cccc(-c4ccccc4-c4cc5ccccc5c5ccccc45)c3)c3cccc4c3oc3c5ccccc5ccc43)cc2)cc1 ; c1ccc(-c2ccc(N(c3ccccc3-c3ccccc3-c3cc4ccccc4c4ccccc34)c3cccc4c3oc3c5ccccc5ccc43)cc2)cc1 ; c1ccc(-c2ccc(N(c3ccc(-c4ccccc4-c4cc5ccccc5c5ccccc45)cc3)c3cccc4c3oc3c5ccccc5ccc43)cc2-c2ccccc2)cc1]

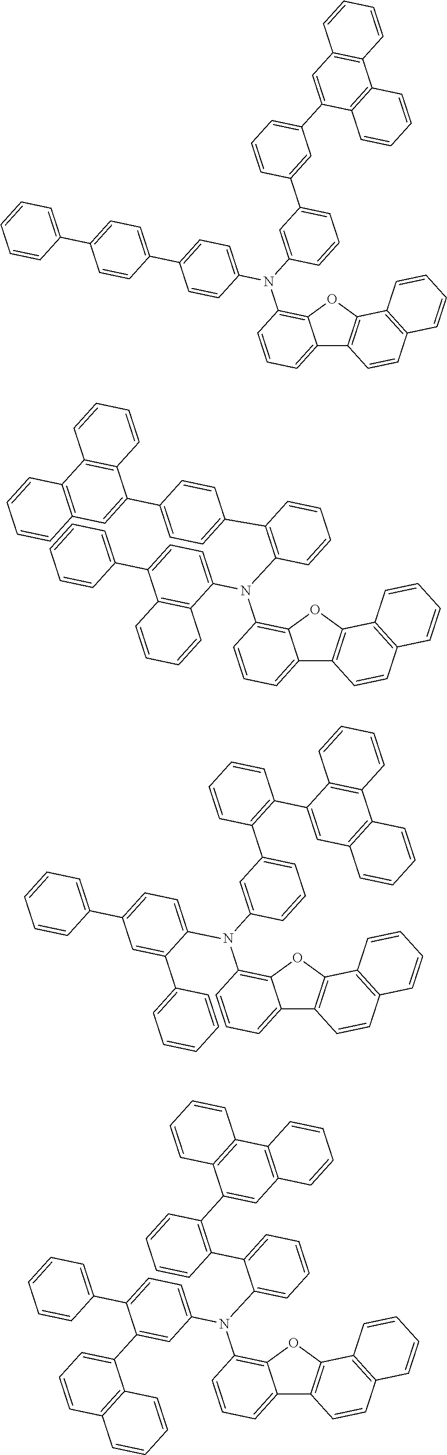

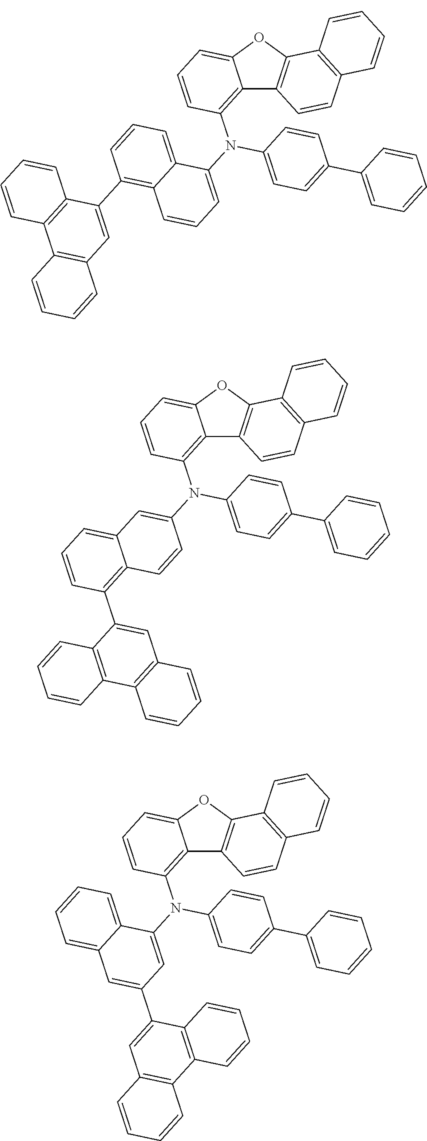

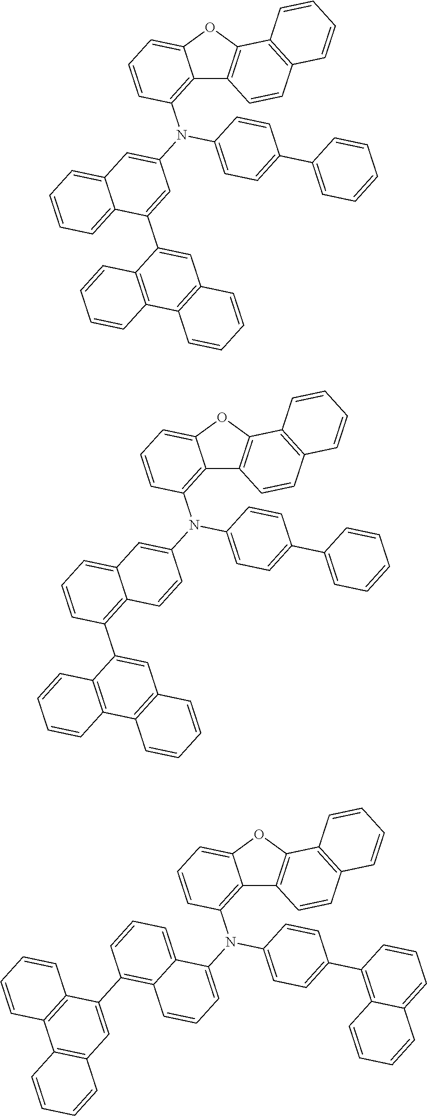 [SMILES: c1ccc(-c2ccc(N(c3cc(-c4cc5ccccc5c5ccccc45)c4ccccc4c3)c3cccc4oc5c6ccccc6ccc5c34)cc2)cc1 ; c1ccc(-c2ccc(N(c3ccc4c(-c5cc6ccccc6c6ccccc56)cccc4c3)c3cccc4oc5c6ccccc6ccc5c34)cc2)cc1 ; c1ccc2c(-c3ccc(N(c4cccc5c(-c6cc7ccccc7c7ccccc67)cccc45)c4cccc5oc6c7ccccc7ccc6c45)cc3)cccc2c1]

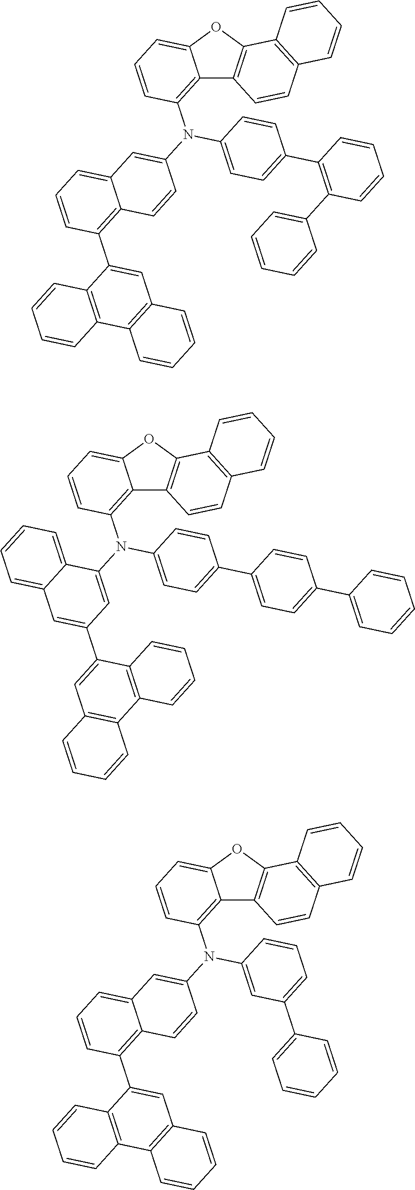 [SMILES: c1ccc(-c2ccccc2-c2ccc(N(c3ccc4c(-c5cc6ccccc6c6ccccc56)cccc4c3)c3cccc4oc5c6ccccc6ccc5c34)cc2)cc1 ; c1ccc(-c2ccc(-c3ccc(N(c4cc(-c5cc6ccccc6c6ccccc56)cc5ccccc45)c4cccc5oc6c7ccccc7ccc6c45)cc3)cc2)cc1 ; c1ccc(-c2cccc(N(c3ccc4c(-c5cc6ccccc6c6ccccc56)cccc4c3)c3cccc4oc5c6ccccc6ccc5c34)c2)cc1]

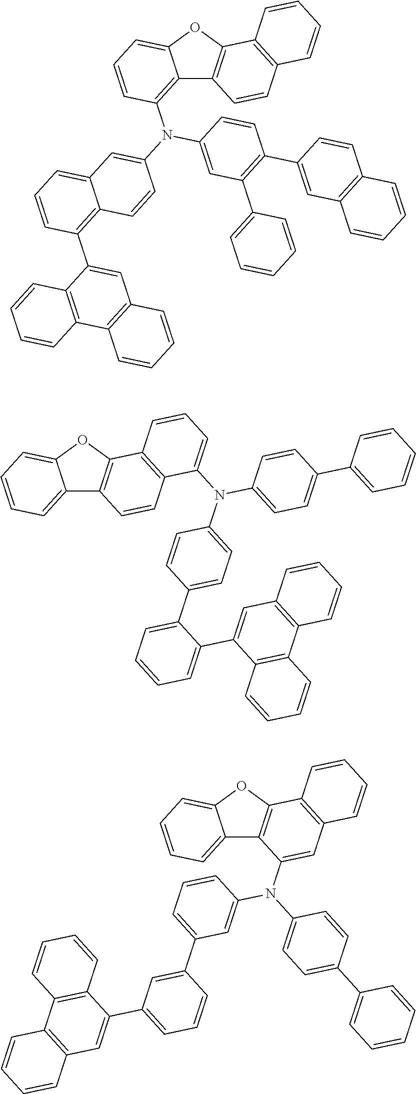 [SMILES: c1ccc(-c2cc(N(c3ccc4c(-c5cc6ccccc6c6ccccc56)cccc4c3)c3cccc4oc5c6ccccc6ccc5c34)ccc2-c2ccc3ccccc3c2)cc1 ; c1ccc(-c2ccc(N(c3ccc(-c4ccccc4-c4cc5ccccc5c5ccccc45)cc3)c3cccc4c3ccc3c5ccccc5oc43)cc2)cc1 ; c1ccc(-c2ccc(N(c3cccc(-c4cccc(-c5cc6ccccc6c6ccccc56)c4)c3)c3cc4ccccc4c4oc5ccccc5c34)cc2)cc1]

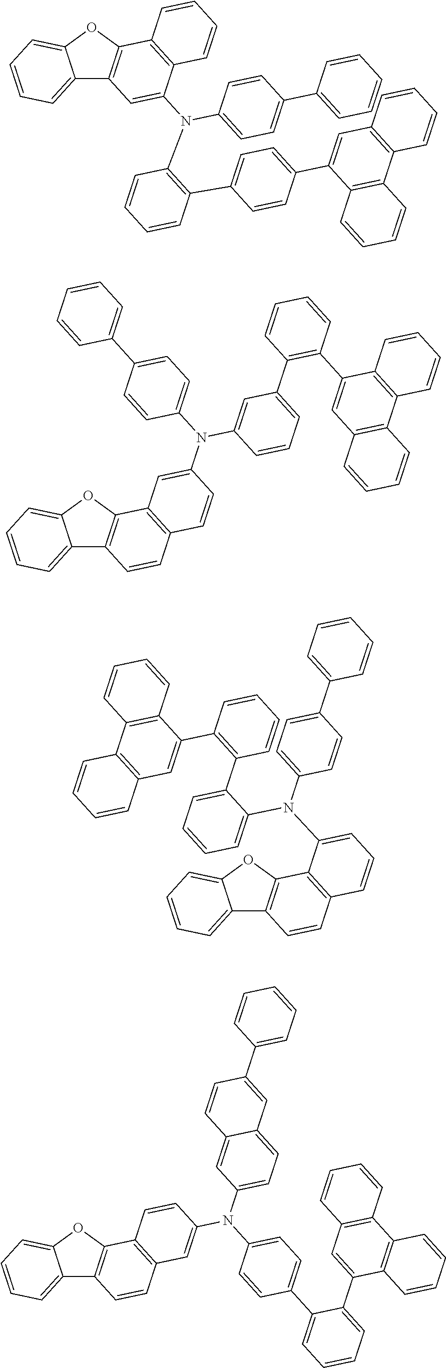 [SMILES: c1ccc(-c2ccc(N(c3ccccc3-c3ccc(-c4cc5ccccc5c5ccccc45)cc3)c3cc4c5ccccc5oc4c4ccccc34)cc2)cc1 ; c1ccc(-c2ccc(N(c3cccc(-c4ccccc4-c4cc5ccccc5c5ccccc45)c3)c3ccc4ccc5c6ccccc6oc5c4c3)cc2)cc1 ; c1ccc(-c2ccc(N(c3ccccc3-c3ccccc3-c3cc4ccccc4c4ccccc34)c3cccc4ccc5c6ccccc6oc5c34)cc2)cc1 ; c1ccc(-c2ccc3cc(N(c4ccc(-c5ccccc5-c5cc6ccccc6c6ccccc56)cc4)c4ccc5c(ccc6c7ccccc7oc56)c4)ccc3c2)cc1]

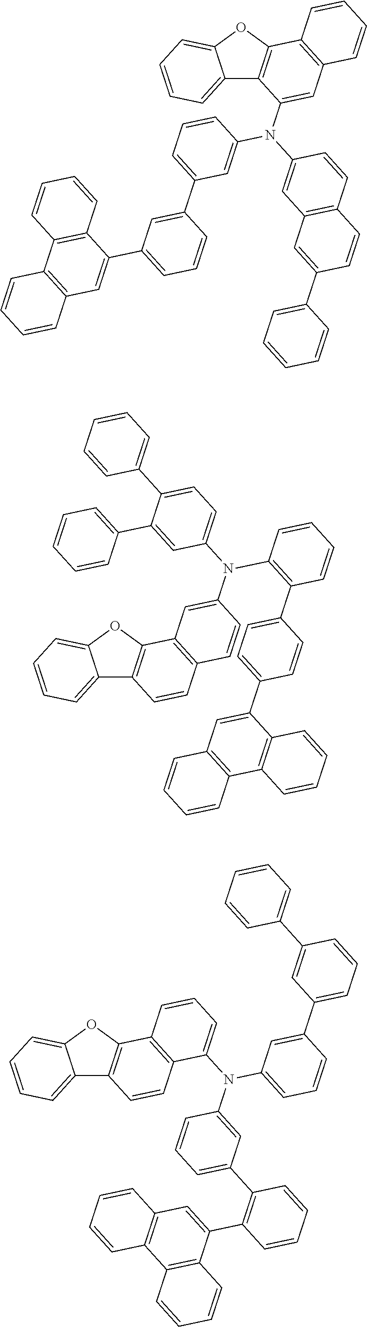 [SMILES: c1ccc(-c2ccc3ccc(N(c4cccc(-c5cccc(-c6cc7ccccc7c7ccccc67)c5)c4)c4cc5ccccc5c5oc6ccccc6c45)cc3c2)cc1 ; c1ccc(-c2ccc(N(c3ccc4ccc5c6ccccc6oc5c4c3)c3ccccc3-c3ccc(-c4cc5ccccc5c5ccccc45)cc3)cc2-c2ccccc2)cc1 ; c1ccc(-c2cccc(-c3cccc(N(c4cccc(-c5ccccc5-c5cc6ccccc6c6ccccc56)c4)c4cccc5c4ccc4c6ccccc6oc54)c3)c2)cc1]

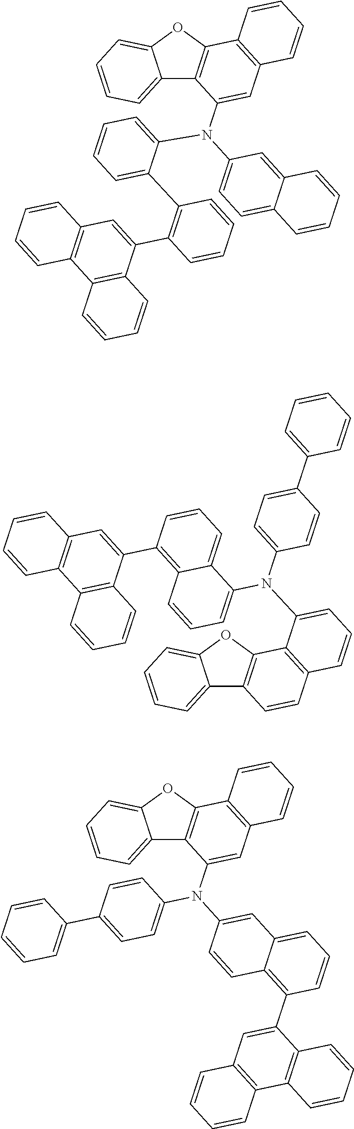 [SMILES: c1ccc(-c2cc3ccccc3c3ccccc23)c(-c2ccccc2N(c2ccc3ccccc3c2)c2cc3ccccc3c3oc4ccccc4c23)c1 ; c1ccc(-c2ccc(N(c3cccc4c(-c5cc6ccccc6c6ccccc56)cccc34)c3cccc4ccc5c6ccccc6oc5c34)cc2)cc1 ; c1ccc(-c2ccc(N(c3ccc4c(-c5cc6ccccc6c6ccccc56)cccc4c3)c3cc4ccccc4c4oc5ccccc5c34)cc2)cc1]

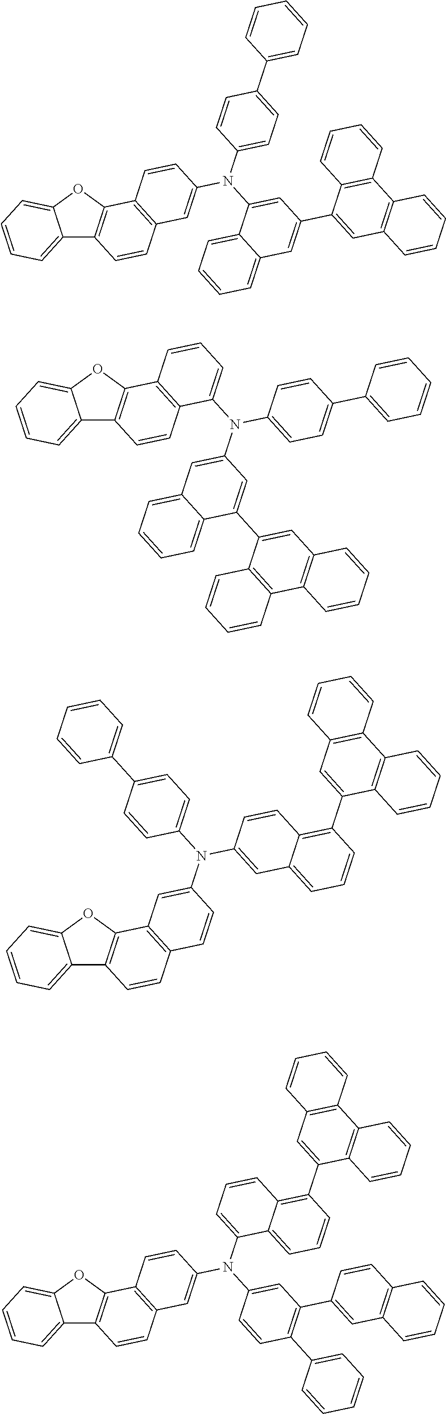

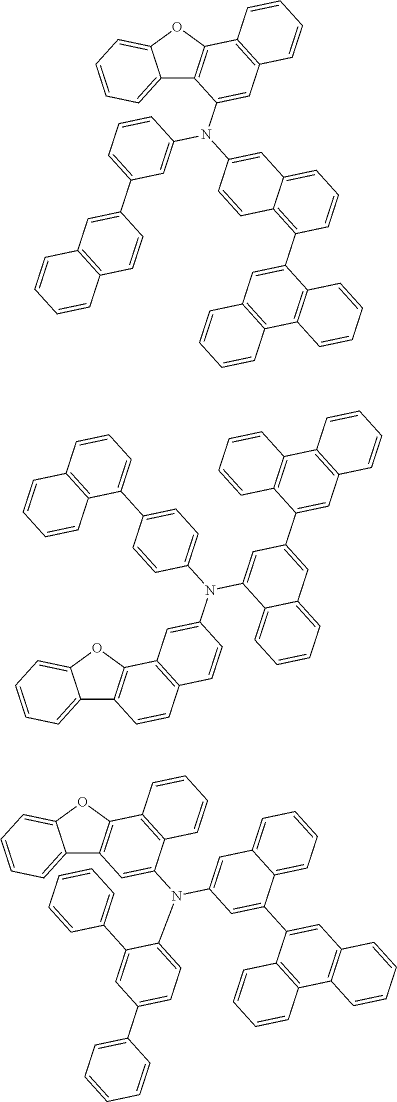

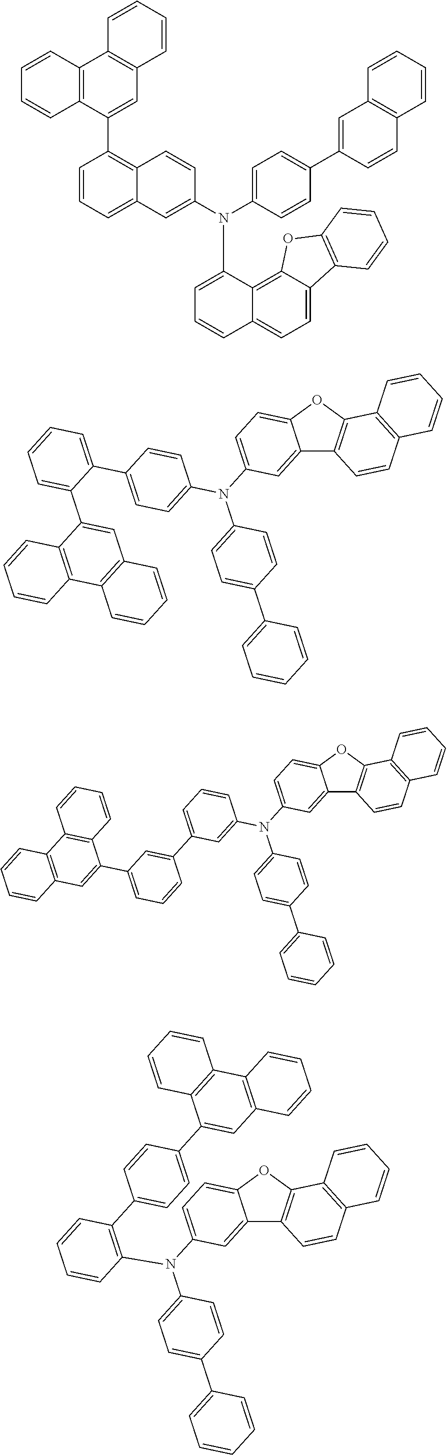

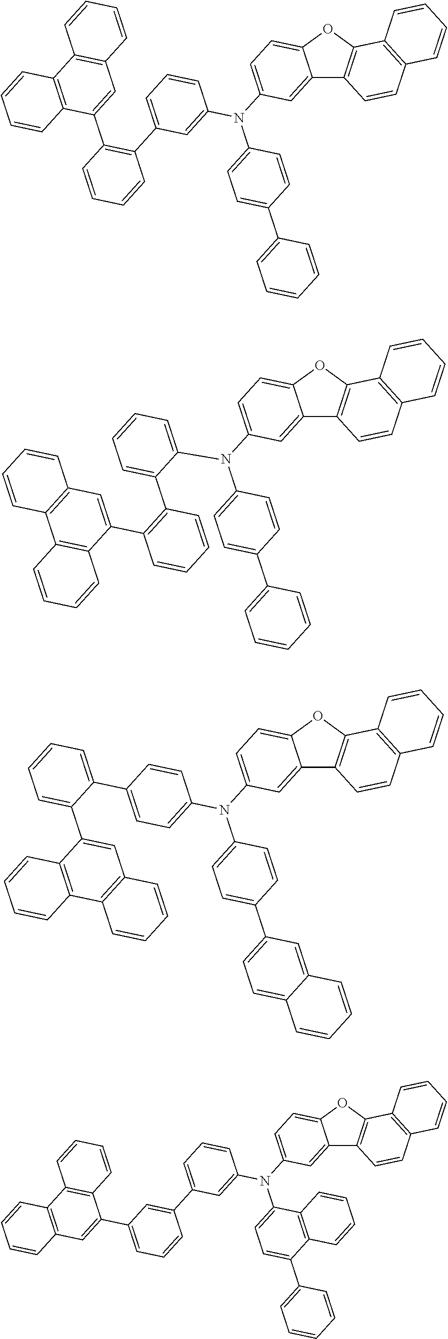

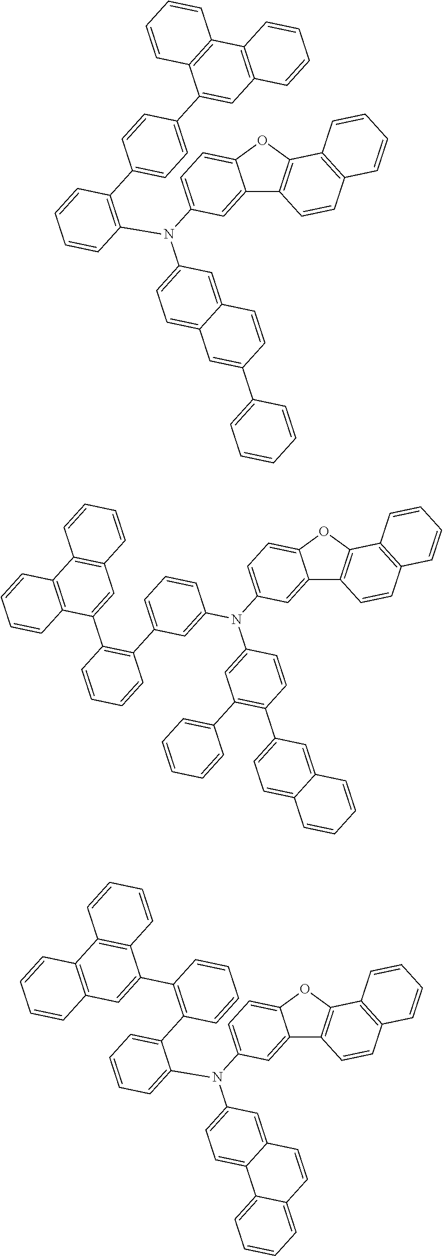

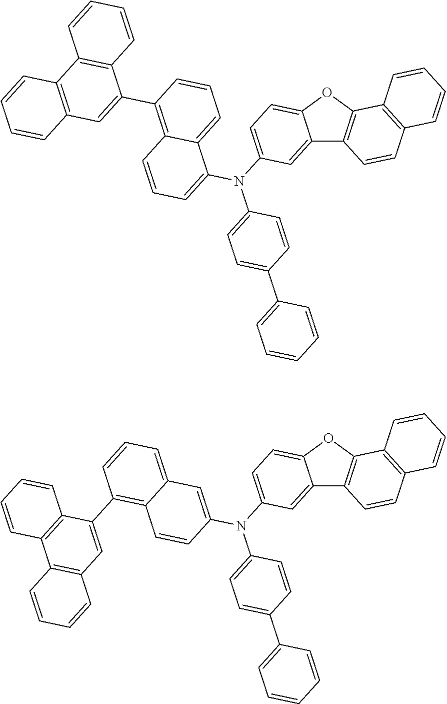

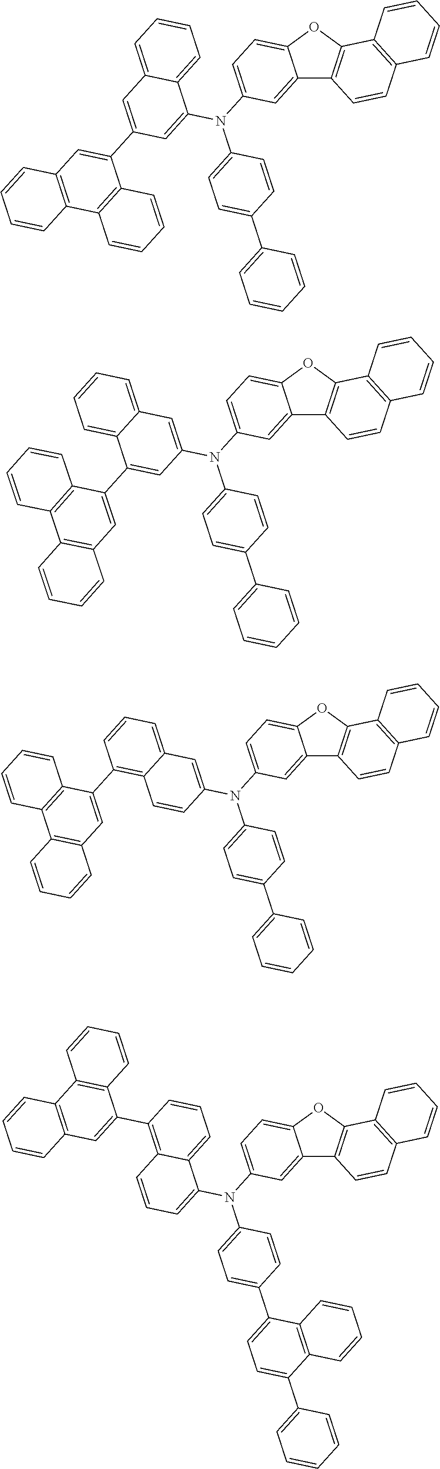 [SMILES: c1ccc(-c2ccc(N(c3ccc4oc5c6ccccc6ccc5c4c3)c3cc(-c4cc5ccccc5c5ccccc45)cc4ccccc34)cc2)cc1 ; c1ccc(-c2ccc(N(c3cc(-c4cc5ccccc5c5ccccc45)c4ccccc4c3)c3ccc4oc5c6ccccc6ccc5c4c3)cc2)cc1 ; c1ccc(-c2ccc(N(c3ccc4c(-c5cc6ccccc6c6ccccc56)cccc4c3)c3ccc4oc5c6ccccc6ccc5c4c3)cc2)cc1 ; c1ccc(-c2ccc(-c3ccc(N(c4ccc5oc6c7ccccc7ccc6c5c4)c4cccc5c(-c6cc7ccccc7c7ccccc67)cccc45)cc3)c3ccccc23)cc1]

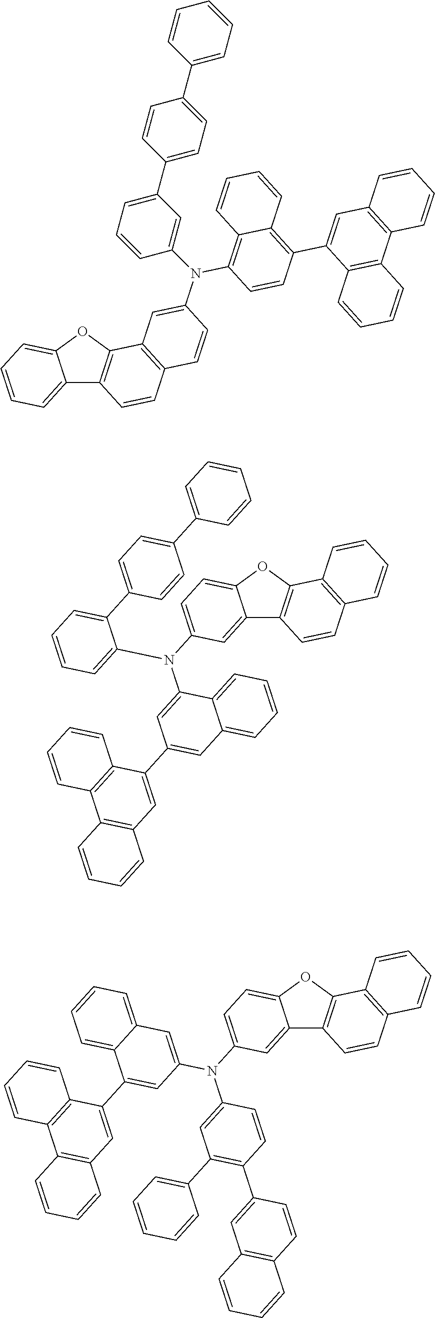 [SMILES: c1ccc(-c2ccc(-c3cccc(N(c4ccc5ccc6c7ccccc7oc6c5c4)c4ccc(-c5cc6ccccc6c6ccccc56)c5ccccc45)c3)cc2)cc1 ; c1ccc(-c2ccc(-c3ccccc3N(c3ccc4oc5c6ccccc6ccc5c4c3)c3cc(-c4cc5ccccc5c5ccccc45)cc4ccccc34)cc2)cc1 ; c1ccc(-c2cc(N(c3cc(-c4cc5ccccc5c5ccccc45)c4ccccc4c3)c3ccc4oc5c6ccccc6ccc5c4c3)ccc2-c2ccc3ccccc3c2)cc1]

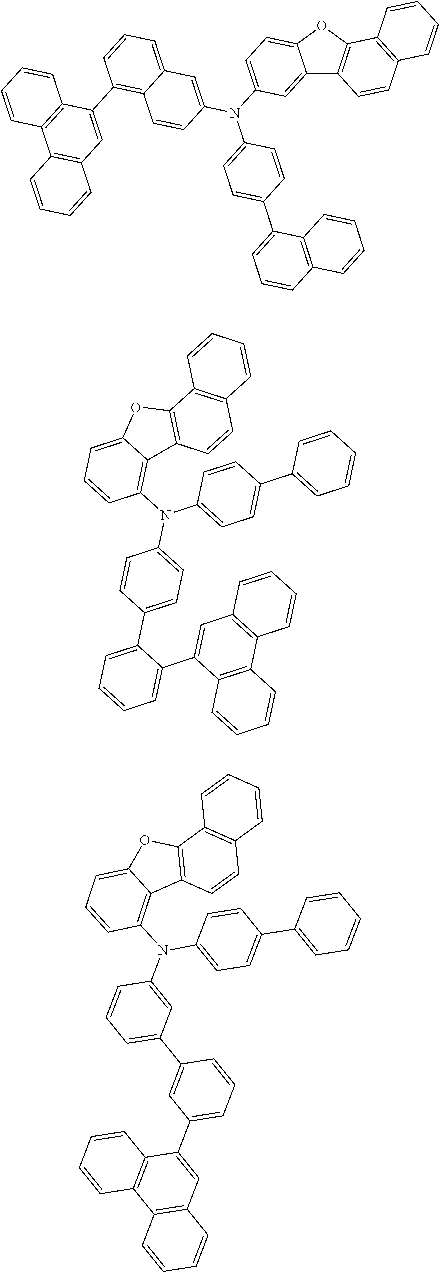

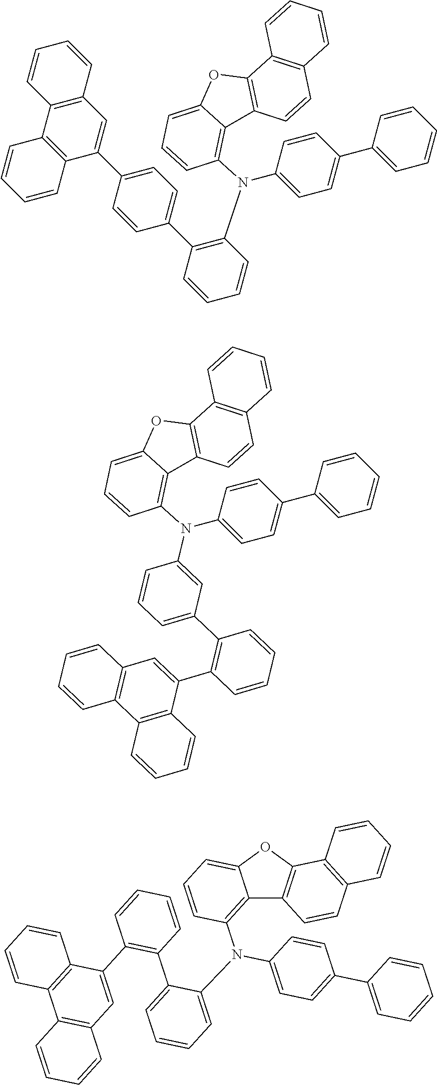 [SMILES: c1ccc(-c2ccc(N(c3ccccc3-c3ccc(-c4cc5ccccc5c5ccccc45)cc3)c3cccc4oc5c6ccccc6ccc5c34)cc2)cc1 ; c1ccc(-c2ccc(N(c3cccc(-c4ccccc4-c4cc5ccccc5c5ccccc45)c3)c3cccc4oc5c6ccccc6ccc5c34)cc2)cc1 ; c1ccc(-c2ccc(N(c3ccccc3-c3ccccc3-c3cc4ccccc4c4ccccc34)c3cccc4oc5c6ccccc6ccc5c34)cc2)cc1]

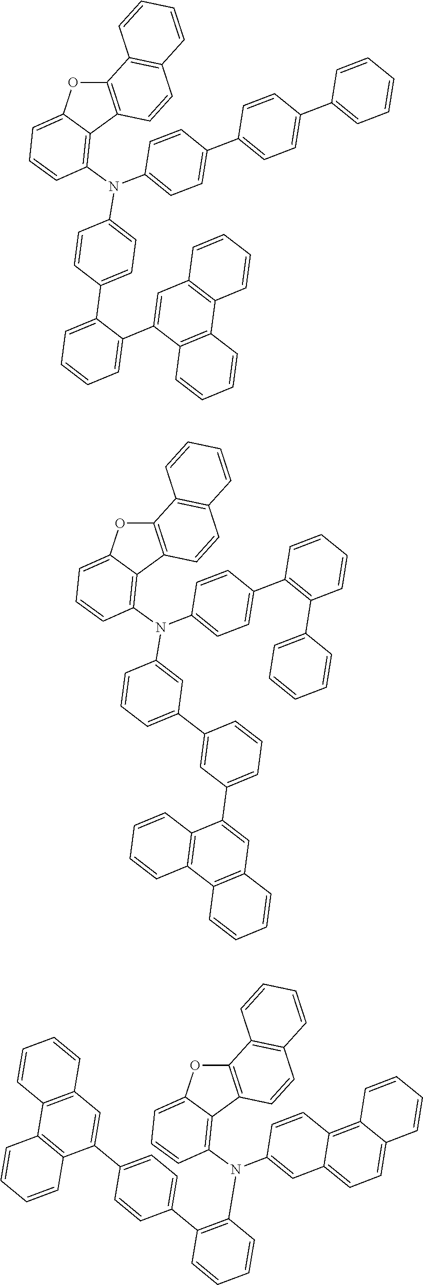

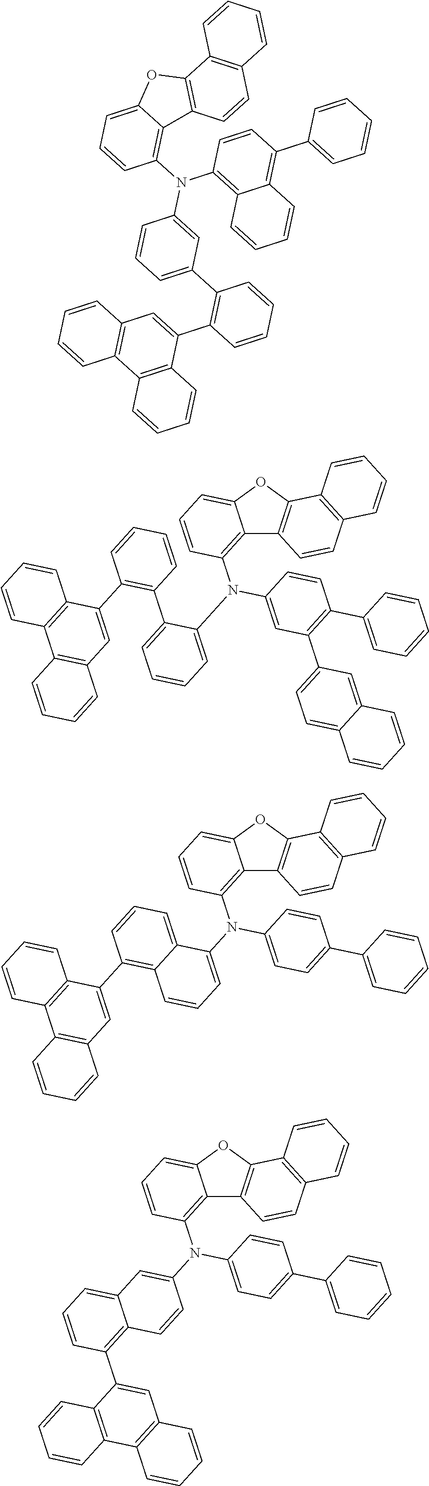

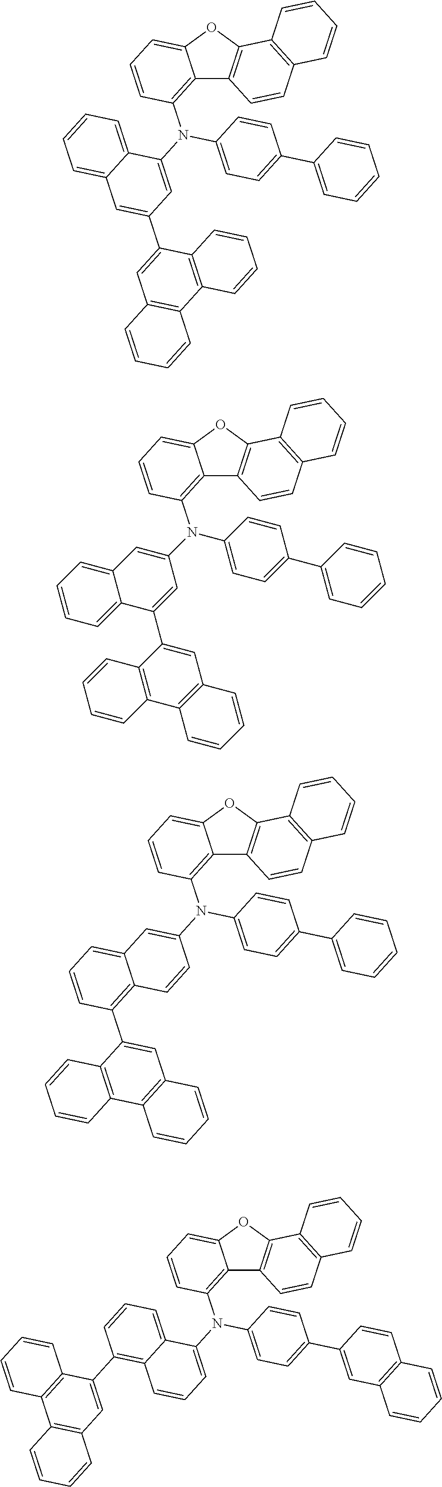 [SMILES: c1ccc(-c2ccc(N(c3cc(-c4cc5ccccc5c5ccccc45)cc4ccccc34)c3cccc4oc5c6ccccc6ccc5c34)cc2)cc1 ; c1ccc(-c2ccc(N(c3cc(-c4cc5ccccc5c5ccccc45)c4ccccc4c3)c3cccc4oc5c6ccccc6ccc5c34)cc2)cc1 ; c1ccc(-c2ccc(N(c3ccc4c(-c5cc6ccccc6c6ccccc56)cccc4c3)c3cccc4oc5c6ccccc6ccc5c34)cc2)cc1 ; c1ccc2cc(-c3ccc(N(c4cccc5c(-c6cc7ccccc7c7ccccc67)cccc45)c4cccc5oc6c7ccccc7ccc6c45)cc3)ccc2c1]

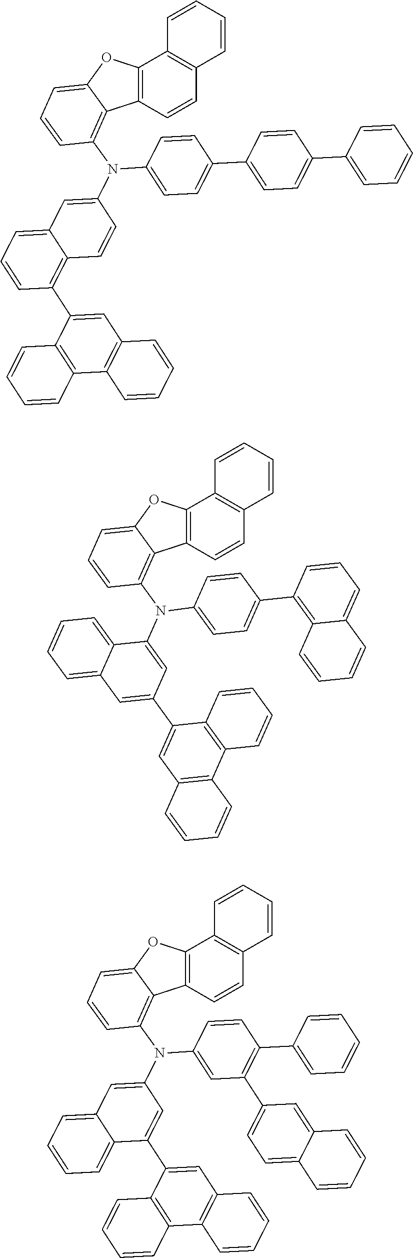 [SMILES: c1ccc(-c2ccc(-c3ccc(N(c4ccc5c(-c6cc7ccccc7c7ccccc67)cccc5c4)c4cccc5oc6c7ccccc7ccc6c45)cc3)cc2)cc1 ; c1ccc2c(-c3ccc(N(c4cc(-c5cc6ccccc6c6ccccc56)cc5ccccc45)c4cccc5oc6c7ccccc7ccc6c45)cc3)cccc2c1 ; c1ccc(-c2ccc(N(c3cc(-c4cc5ccccc5c5ccccc45)c4ccccc4c3)c3cccc4oc5c6ccccc6ccc5c34)cc2-c2ccc3ccccc3c2)cc1]

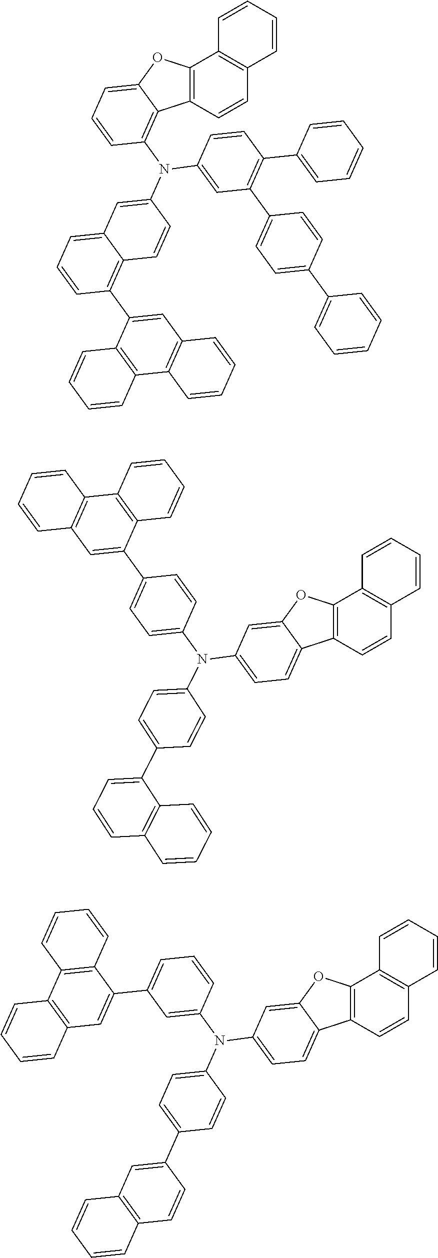 [SMILES: c1ccc(-c2ccc(-c3cc(N(c4ccc5c(-c6cc7ccccc7c7ccccc67)cccc5c4)c4cccc5oc6c7ccccc7ccc6c45)ccc3-c3ccccc3)cc2)cc1 ; c1ccc2c(-c3ccc(N(c4ccc(-c5cc6ccccc6c6ccccc56)cc4)c4ccc5c(c4)oc4c6ccccc6ccc54)cc3)cccc2c1 ; c1cc(-c2cc3ccccc3c3ccccc23)cc(N(c2ccc(-c3ccc4ccccc4c3)cc2)c2ccc3c(c2)oc2c4ccccc4ccc32)c1]

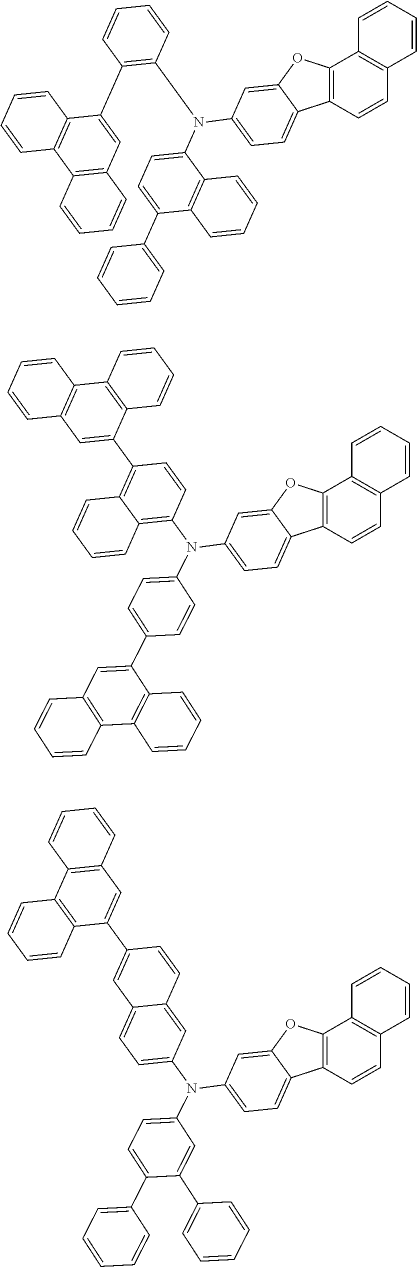 [SMILES: c1ccc(-c2ccc(N(c3ccc4c(c3)oc3c5ccccc5ccc43)c3ccccc3-c3cc4ccccc4c4ccccc34)c3ccccc23)cc1 ; c1ccc2c(c1)cc(-c1ccc(N(c3ccc4c(c3)oc3c5ccccc5ccc43)c3ccc(-c4cc5ccccc5c5ccccc45)c4ccccc34)cc1)c1ccccc12 ; c1ccc(-c2ccc(N(c3ccc4cc(-c5cc6ccccc6c6ccccc56)ccc4c3)c3ccc4c(c3)oc3c5ccccc5ccc43)cc2-c2ccccc2)cc1]

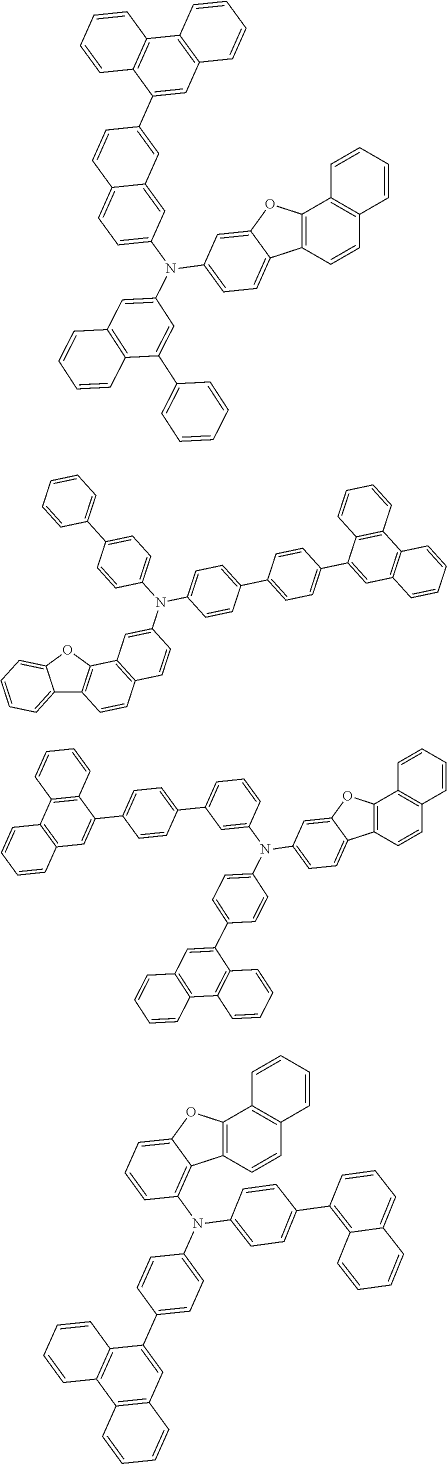 [SMILES: c1ccc(-c2cc(N(c3ccc4ccc(-c5cc6ccccc6c6ccccc56)cc4c3)c3ccc4c(c3)oc3c5ccccc5ccc43)cc3ccccc23)cc1 ; c1ccc(-c2ccc(N(c3ccc(-c4ccc(-c5cc6ccccc6c6ccccc56)cc4)cc3)c3ccc4ccc5c6ccccc6oc5c4c3)cc2)cc1 ; c1cc(-c2ccc(-c3cc4ccccc4c4ccccc34)cc2)cc(N(c2ccc(-c3cc4ccccc4c4ccccc34)cc2)c2ccc3c(c2)oc2c4ccccc4ccc32)c1 ; c1ccc2c(-c3ccc(N(c4ccc(-c5cc6ccccc6c6ccccc56)cc4)c4cccc5oc6c7ccccc7ccc6c45)cc3)cccc2c1]

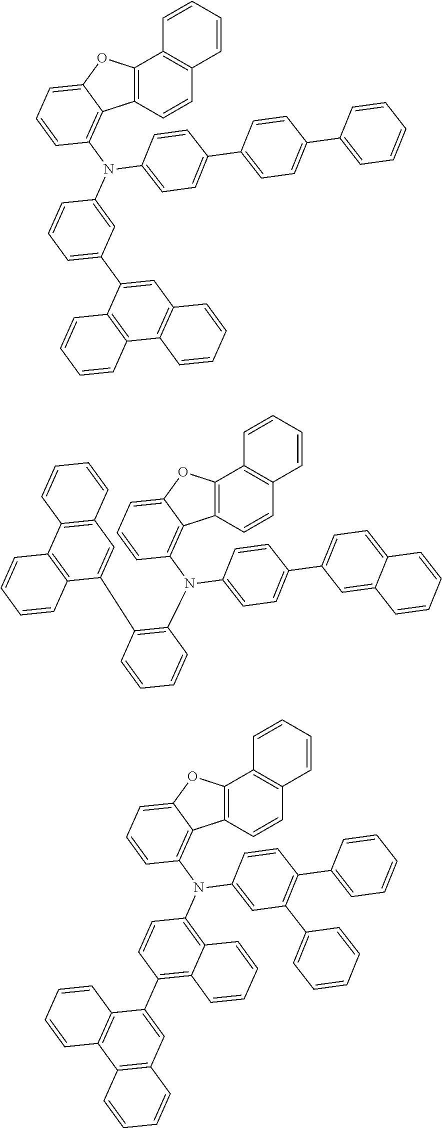 [SMILES: c1ccc(-c2ccc(-c3ccc(N(c4cccc(-c5cc6ccccc6c6ccccc56)c4)c4cccc5oc6c7ccccc7ccc6c45)cc3)cc2)cc1 ; c1ccc(N(c2ccc(-c3ccc4ccccc4c3)cc2)c2cccc3oc4c5ccccc5ccc4c23)c(-c2cc3ccccc3c3ccccc23)c1 ; c1ccc(-c2ccc(N(c3ccc(-c4cc5ccccc5c5ccccc45)c4ccccc34)c3cccc4oc5c6ccccc6ccc5c34)cc2-c2ccccc2)cc1]

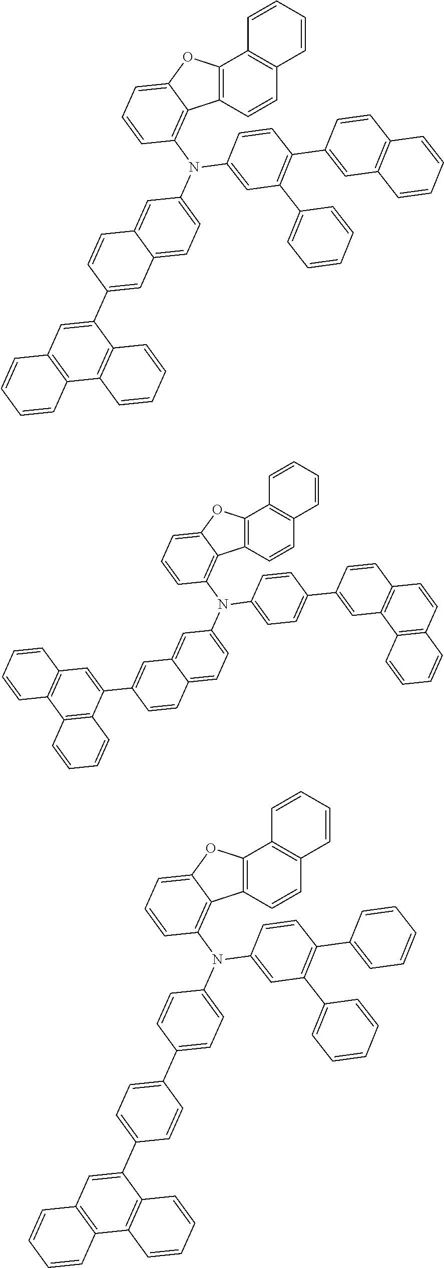 [SMILES: c1ccc(-c2cc(N(c3ccc4cc(-c5cc6ccccc6c6ccccc56)ccc4c3)c3cccc4oc5c6ccccc6ccc5c34)ccc2-c2ccc3ccccc3c2)cc1 ; c1ccc2c(c1)ccc1ccc(-c3ccc(N(c4ccc5ccc(-c6cc7ccccc7c7ccccc67)cc5c4)c4cccc5oc6c7ccccc7ccc6c45)cc3)cc12 ; c1ccc(-c2ccc(N(c3ccc(-c4ccc(-c5cc6ccccc6c6ccccc56)cc4)cc3)c3cccc4oc5c6ccccc6ccc5c34)cc2-c2ccccc2)cc1]

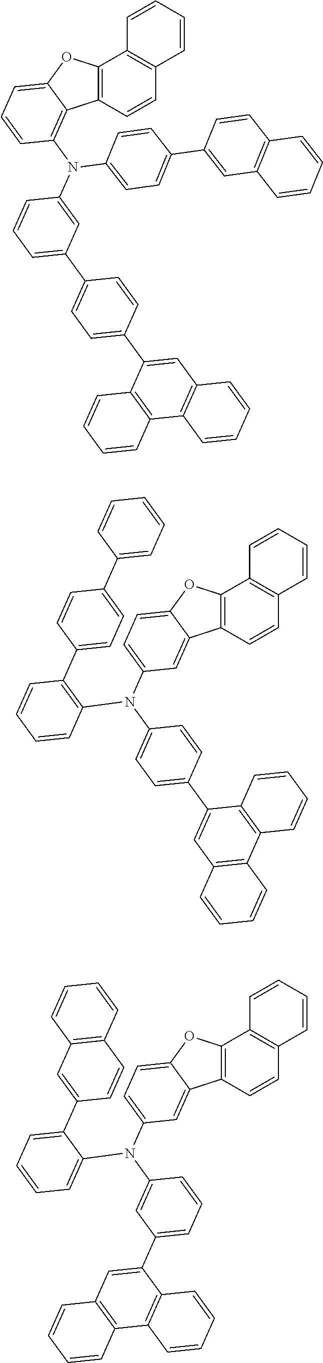 [SMILES: c1cc(-c2ccc(-c3cc4ccccc4c4ccccc34)cc2)cc(N(c2ccc(-c3ccc4ccccc4c3)cc2)c2cccc3oc4c5ccccc5ccc4c23)c1 ; c1ccc(-c2ccc(-c3ccccc3N(c3ccc(-c4cc5ccccc5c5ccccc45)cc3)c3ccc4oc5c6ccccc6ccc5c4c3)cc2)cc1 ; c1cc(-c2cc3ccccc3c3ccccc23)cc(N(c2ccc3oc4c5ccccc5ccc4c3c2)c2ccccc2-c2ccc3ccccc3c2)c1]

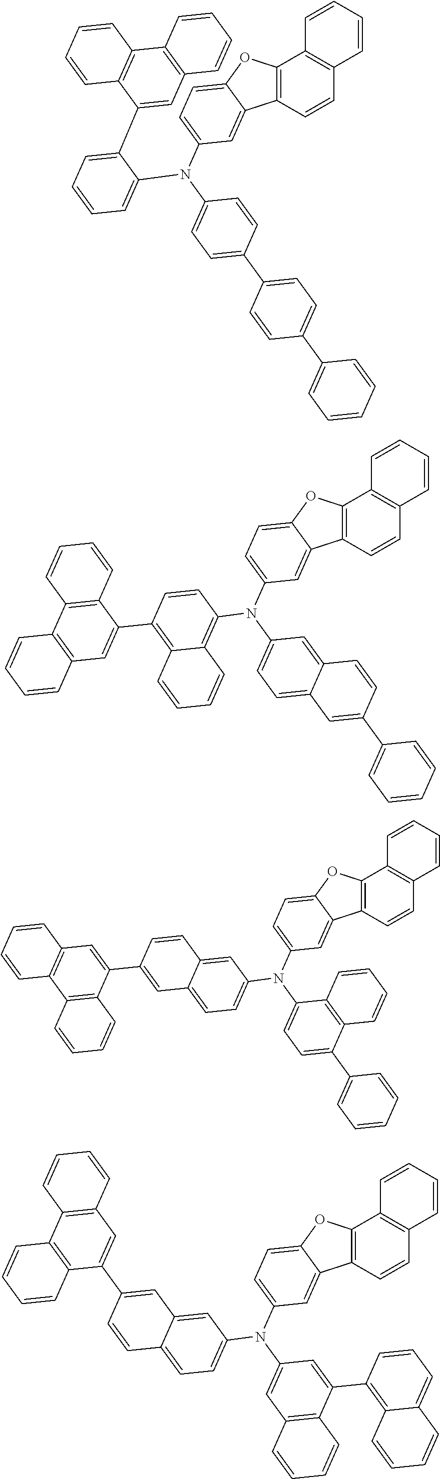

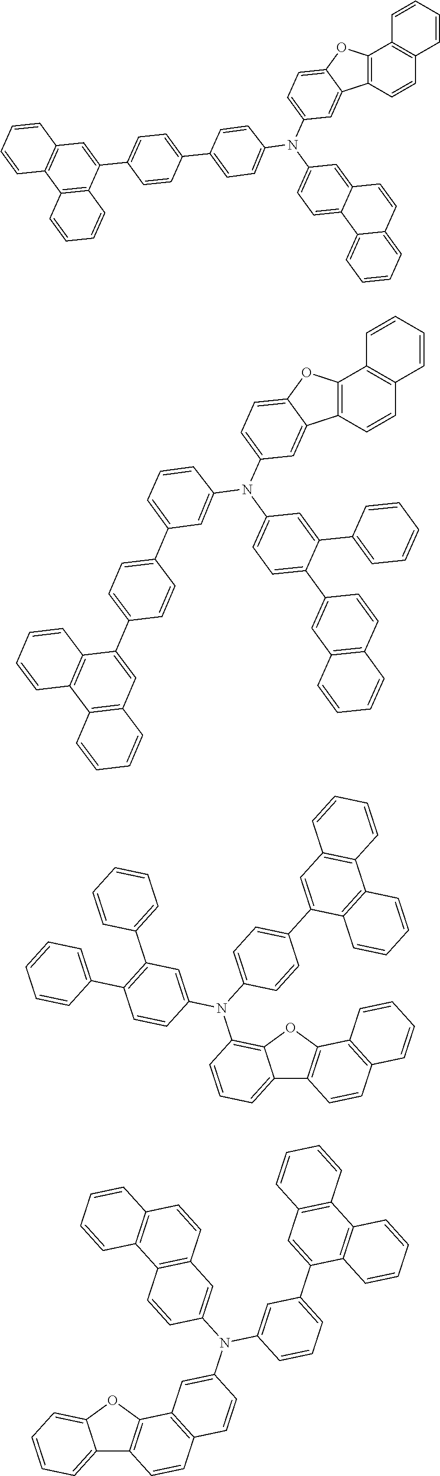

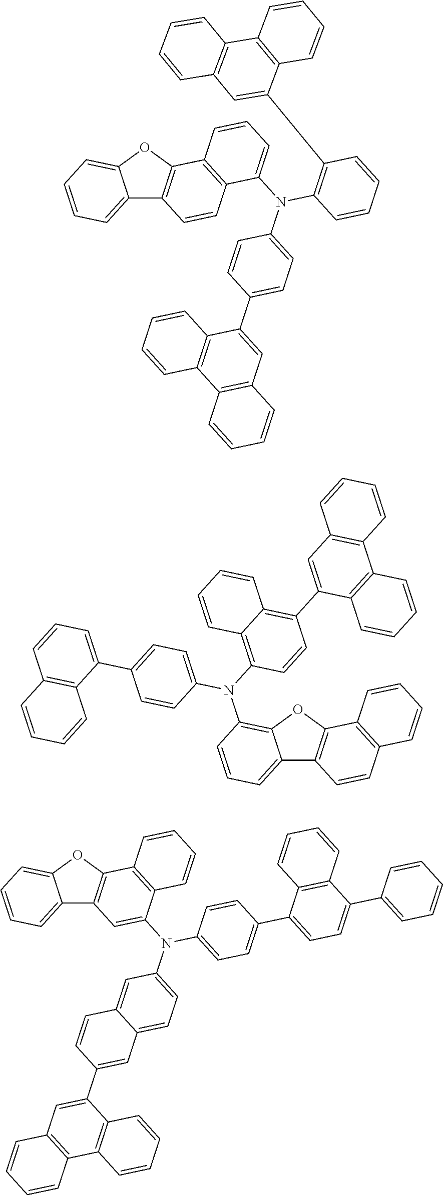 [SMILES: c1ccc(N(c2ccc(-c3cc4ccccc4c4ccccc34)cc2)c2cccc3c2ccc2c4ccccc4oc32)c(-c2cc3ccccc3c3ccccc23)c1 ; c1ccc2c(-c3ccc(N(c4ccc(-c5cc6ccccc6c6ccccc56)c5ccccc45)c4cccc5c4oc4c6ccccc6ccc54)cc3)cccc2c1 ; c1ccc(-c2ccc(-c3ccc(N(c4ccc5cc(-c6cc7ccccc7c7ccccc67)ccc5c4)c4cc5c6ccccc6oc5c5ccccc45)cc3)c3ccccc23)cc1]

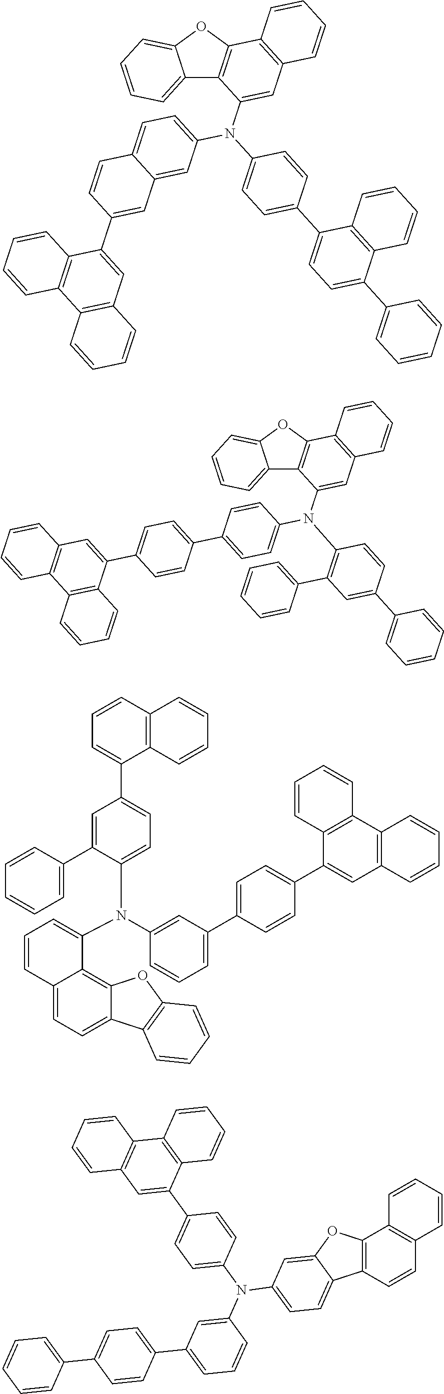

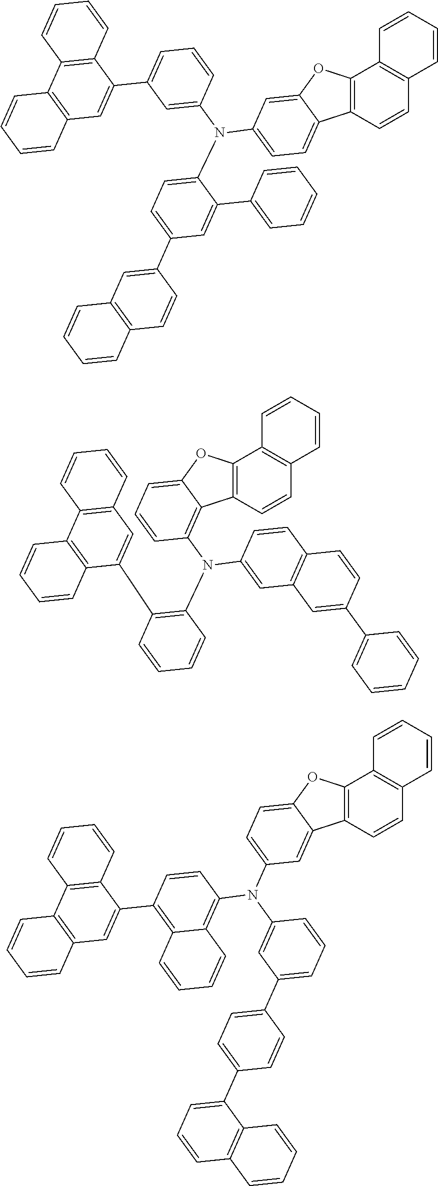

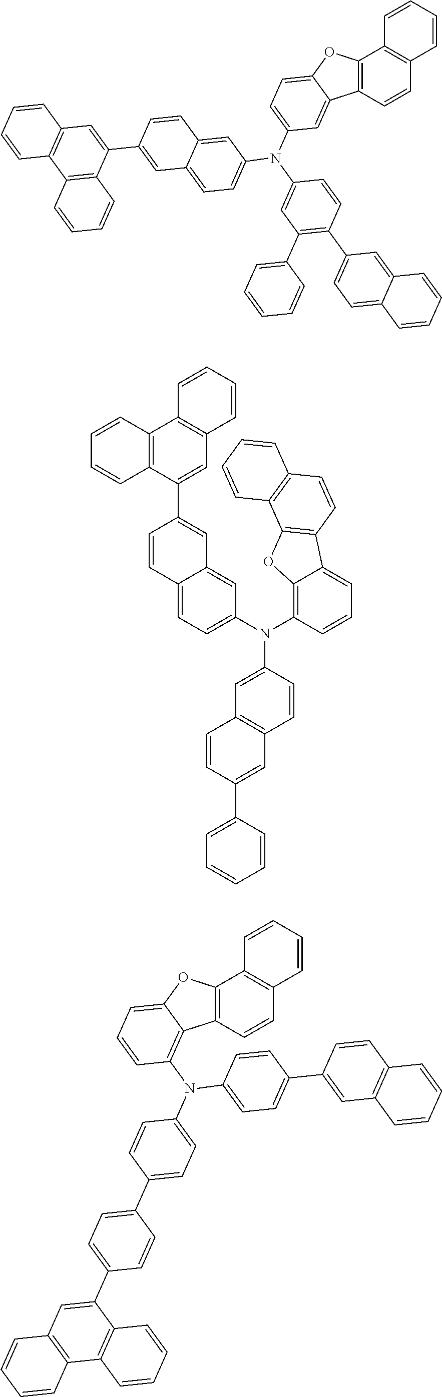 [SMILES: c1ccc(-c2cc(N(c3ccc4cc(-c5cc6ccccc6c6ccccc56)ccc4c3)c3ccc4oc5c6ccccc6ccc5c4c3)ccc2-c2ccc3ccccc3c2)cc1 ; c1ccc(-c2ccc3cc(N(c4ccc5ccc(-c6cc7ccccc7c7ccccc67)cc5c4)c4cccc5c4oc4c6ccccc6ccc54)ccc3c2)cc1 ; c1ccc2cc(-c3ccc(N(c4ccc(-c5ccc(-c6cc7ccccc7c7ccccc67)cc5)cc4)c4cccc5oc6c7ccccc7ccc6c45)cc3)ccc2c1]

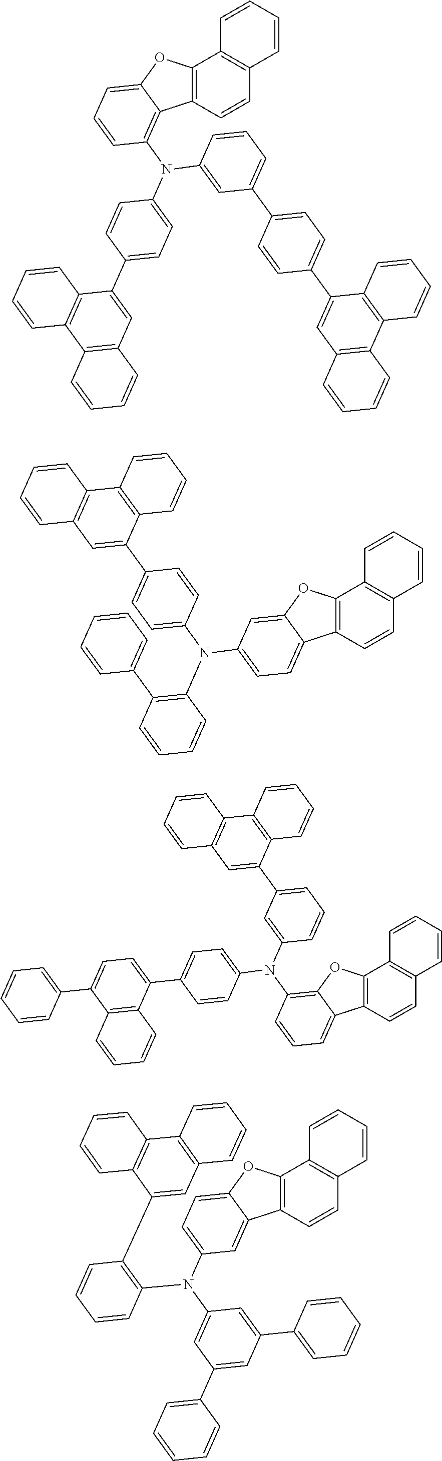

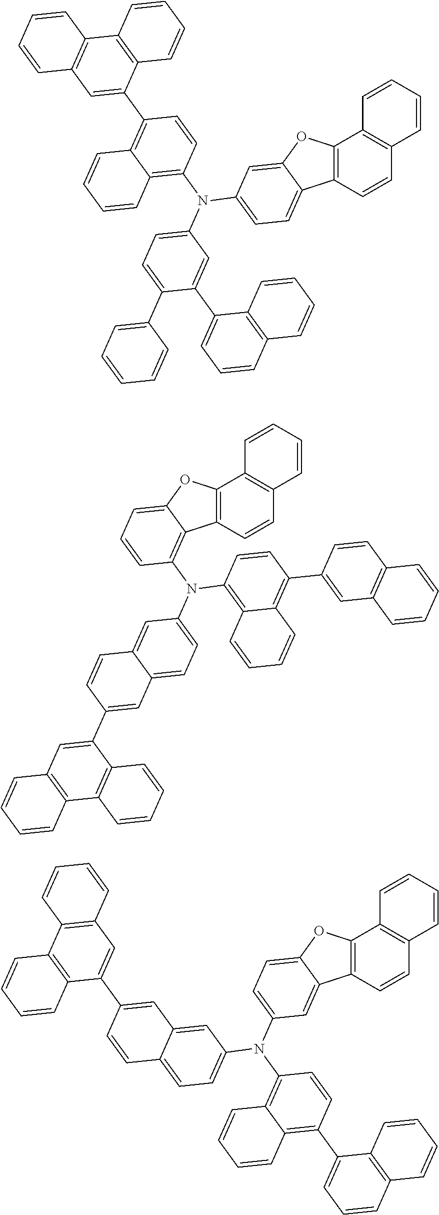

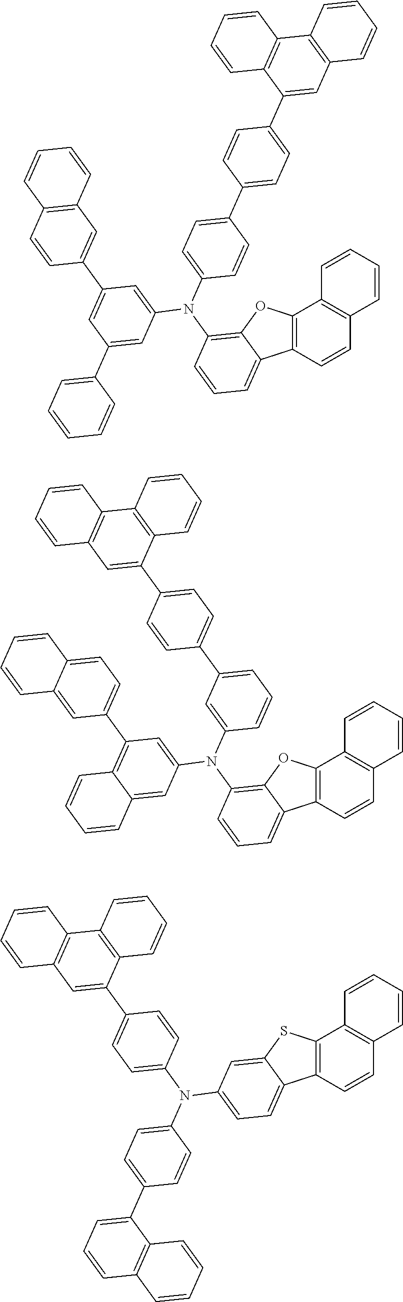 [SMILES: c1ccc(-c2cc(-c3ccc4ccccc4c3)cc(N(c3ccc(-c4ccc(-c5cc6ccccc6c6ccccc56)cc4)cc3)c3cccc4c3oc3c5ccccc5ccc43)c2)cc1 ; c1cc(-c2ccc(-c3cc4ccccc4c4ccccc34)cc2)cc(N(c2cc(-c3ccc4ccccc4c3)c3ccccc3c2)c2cccc3c2oc2c4ccccc4ccc32)c1 ; c1ccc2c(-c3ccc(N(c4ccc(-c5cc6ccccc6c6ccccc56)cc4)c4ccc5c(c4)sc4c6ccccc6ccc54)cc3)cccc2c1]

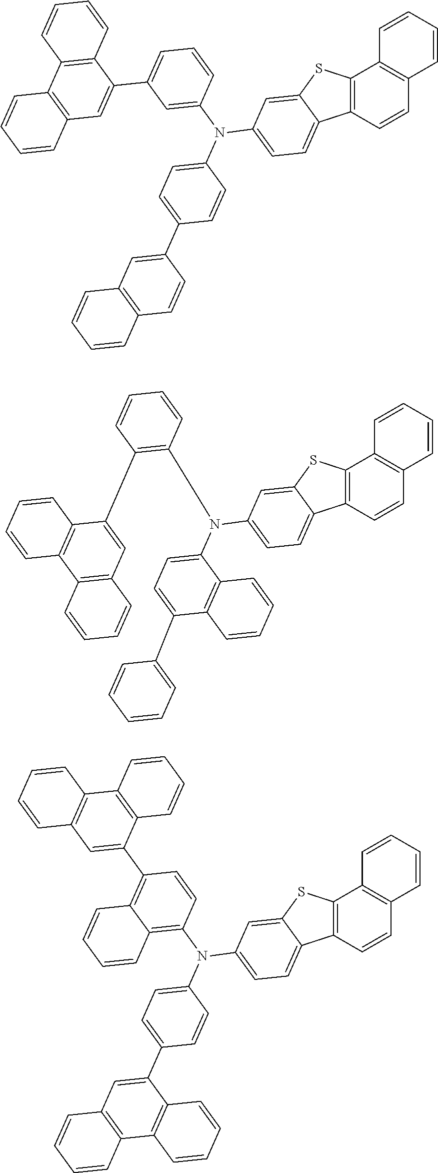

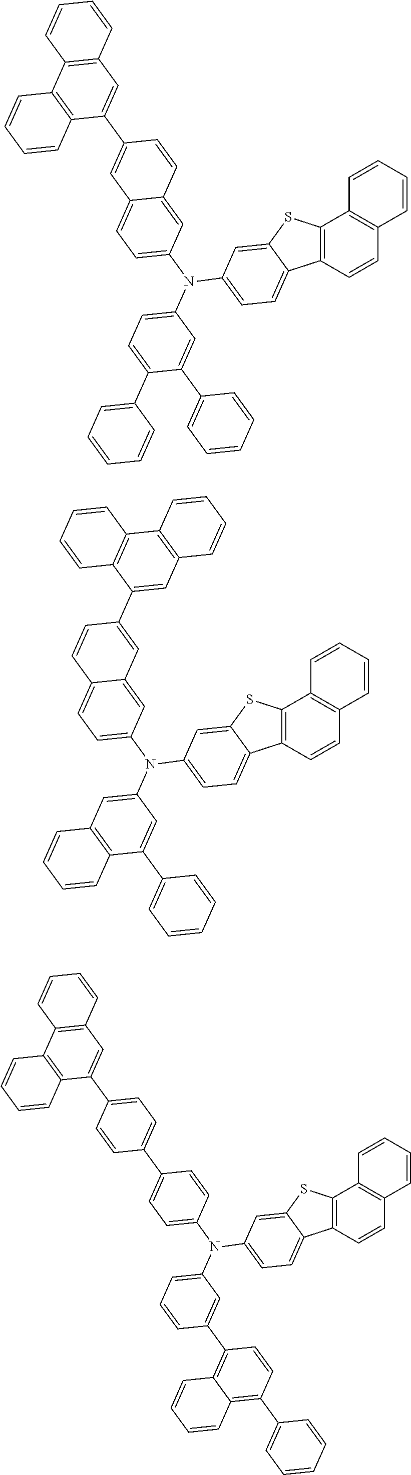

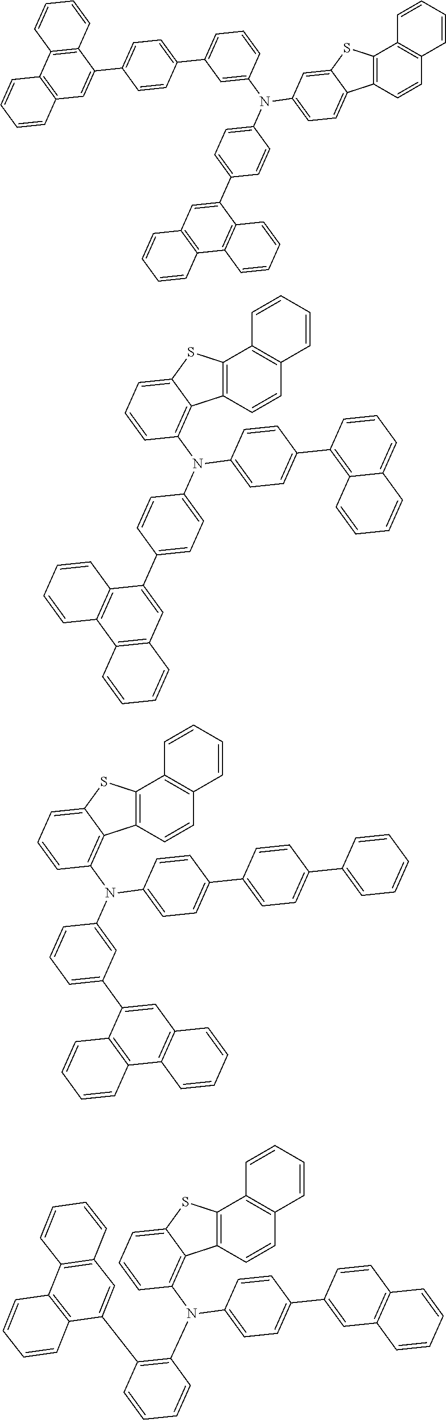

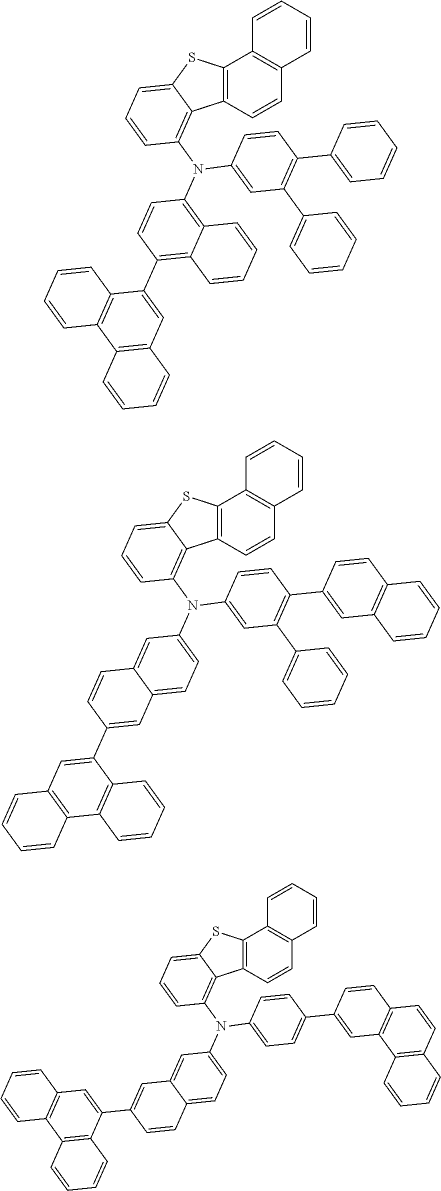 [SMILES: c1ccc(-c2ccc(N(c3ccc(-c4cc5ccccc5c5ccccc45)c4ccccc34)c3cccc4sc5c6ccccc6ccc5c34)cc2-c2ccccc2)cc1 ; c1ccc(-c2cc(N(c3ccc4cc(-c5cc6ccccc6c6ccccc56)ccc4c3)c3cccc4sc5c6ccccc6ccc5c34)ccc2-c2ccc3ccccc3c2)cc1 ; c1ccc2c(c1)ccc1ccc(-c3ccc(N(c4ccc5ccc(-c6cc7ccccc7c7ccccc67)cc5c4)c4cccc5sc6c7ccccc7ccc6c45)cc3)cc12]

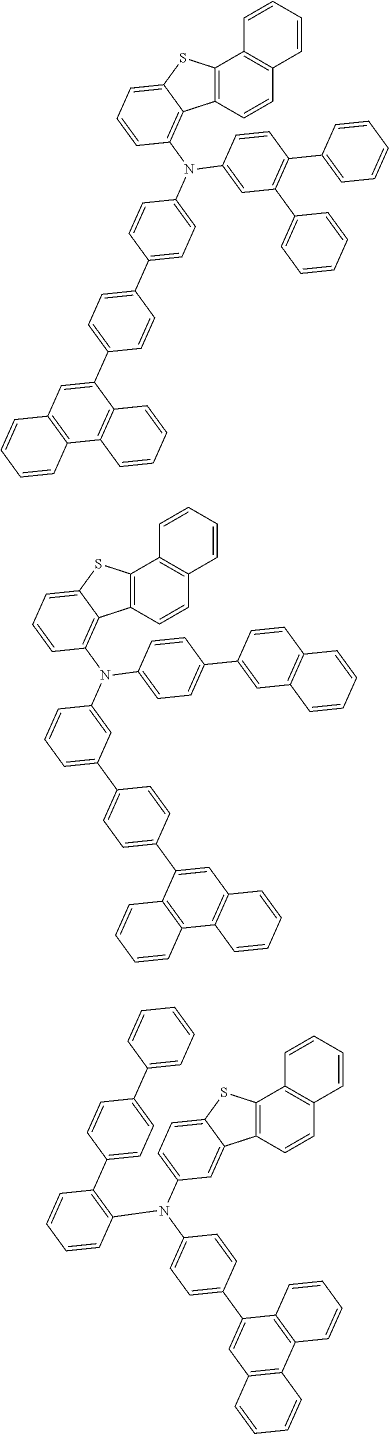 [SMILES: c1ccc(-c2ccc(N(c3ccc(-c4ccc(-c5cc6ccccc6c6ccccc56)cc4)cc3)c3cccc4sc5c6ccccc6ccc5c34)cc2-c2ccccc2)cc1 ; c1cc(-c2ccc(-c3cc4ccccc4c4ccccc34)cc2)cc(N(c2ccc(-c3ccc4ccccc4c3)cc2)c2cccc3sc4c5ccccc5ccc4c23)c1 ; c1ccc(-c2ccc(-c3ccccc3N(c3ccc(-c4cc5ccccc5c5ccccc45)cc3)c3ccc4sc5c6ccccc6ccc5c4c3)cc2)cc1]

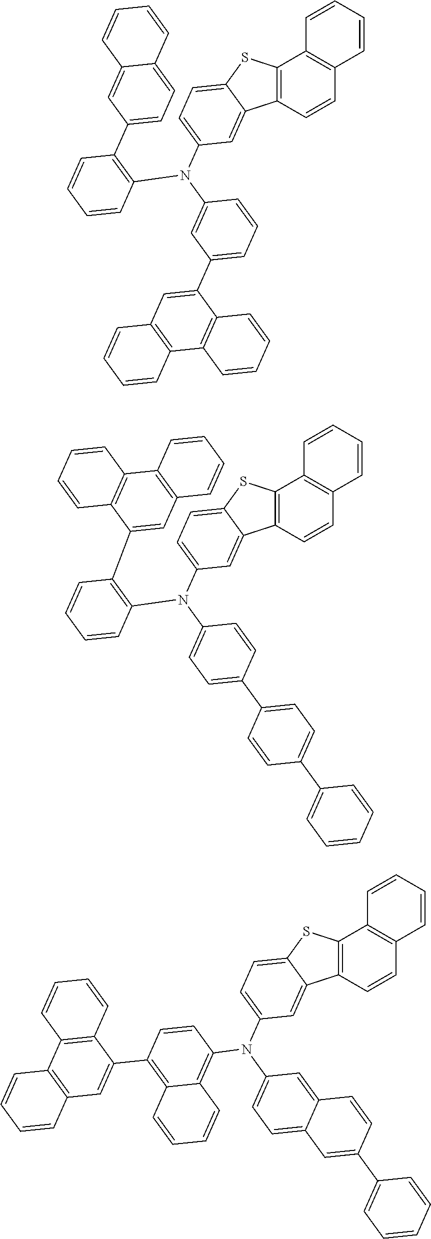 [SMILES: c1cc(-c2cc3ccccc3c3ccccc23)cc(N(c2ccc3sc4c5ccccc5ccc4c3c2)c2ccccc2-c2ccc3ccccc3c2)c1 ; c1ccc(-c2ccc(-c3ccc(N(c4ccc5sc6c7ccccc7ccc6c5c4)c4ccccc4-c4cc5ccccc5c5ccccc45)cc3)cc2)cc1 ; c1ccc(-c2ccc3cc(N(c4ccc5sc6c7ccccc7ccc6c5c4)c4ccc(-c5cc6ccccc6c6ccccc56)c5ccccc45)ccc3c2)cc1]

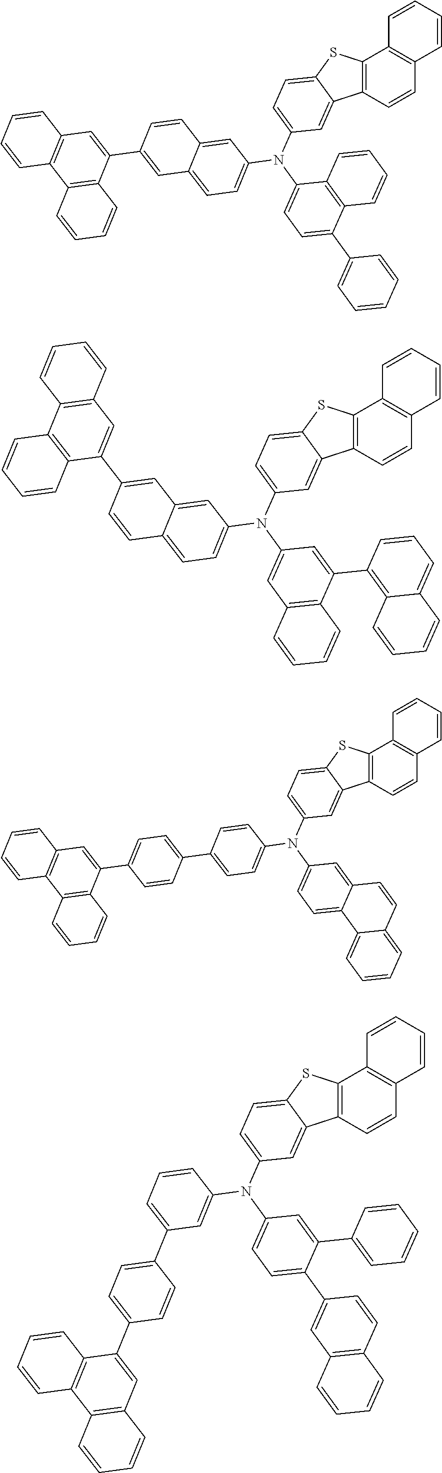

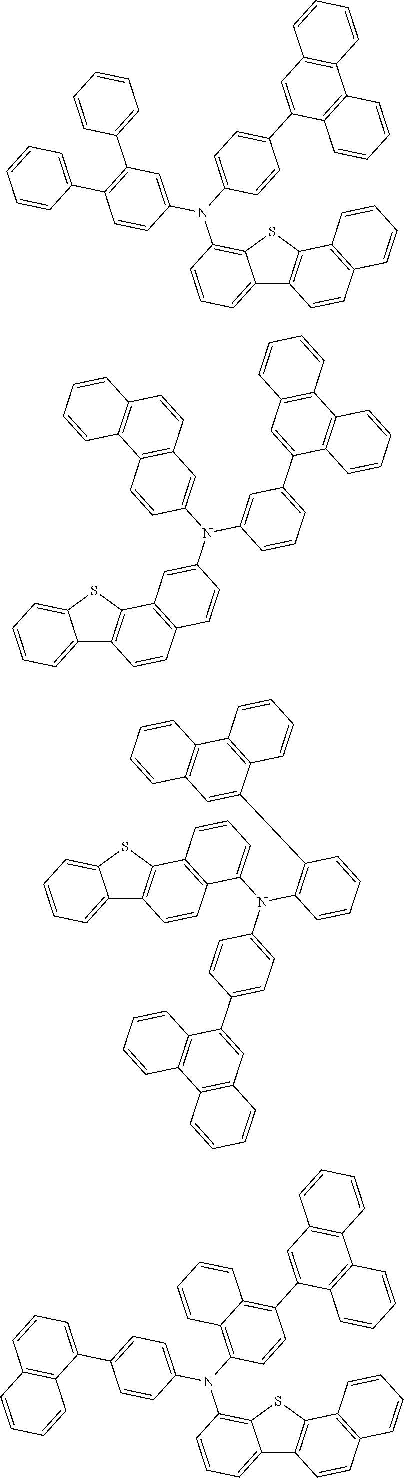

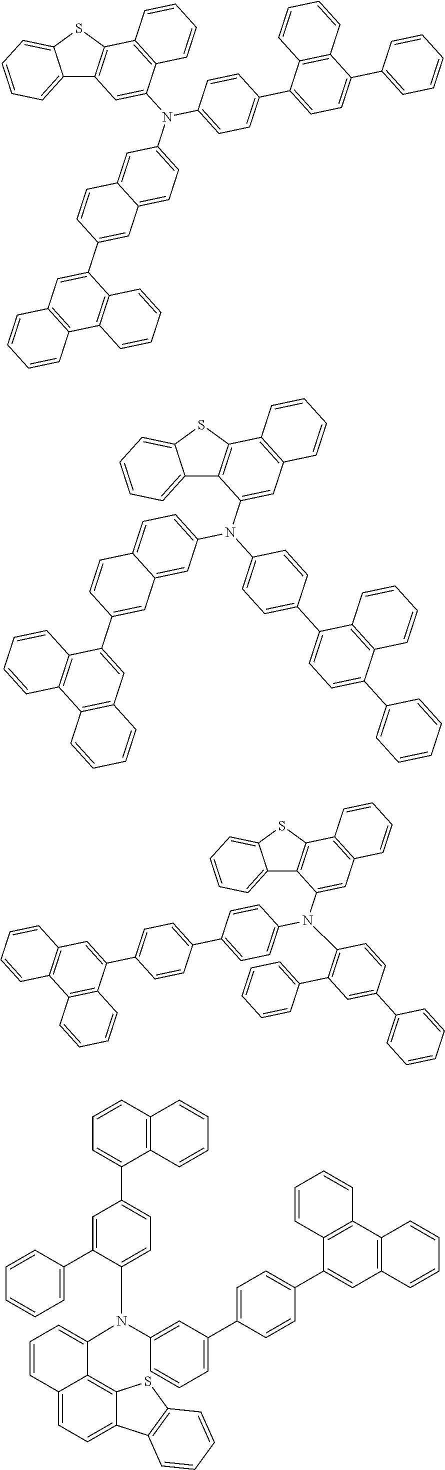 [SMILES: c1ccc(-c2ccc(-c3ccc(N(c4ccc5cc(-c6cc7ccccc7c7ccccc67)ccc5c4)c4cc5c6ccccc6sc5c5ccccc45)cc3)c3ccccc23)cc1 ; c1ccc(-c2ccc(-c3ccc(N(c4ccc5ccc(-c6cc7ccccc7c7ccccc67)cc5c4)c4cc5ccccc5c5sc6ccccc6c45)cc3)c3ccccc23)cc1 ; c1ccc(-c2ccc(N(c3ccc(-c4ccc(-c5cc6ccccc6c6ccccc56)cc4)cc3)c3cc4ccccc4c4sc5ccccc5c34)c(-c3ccccc3)c2)cc1 ; c1ccc(-c2cc(-c3cccc4ccccc34)ccc2N(c2cccc(-c3ccc(-c4cc5ccccc5c5ccccc45)cc3)c2)c2cccc3ccc4c5ccccc5sc4c23)cc1]

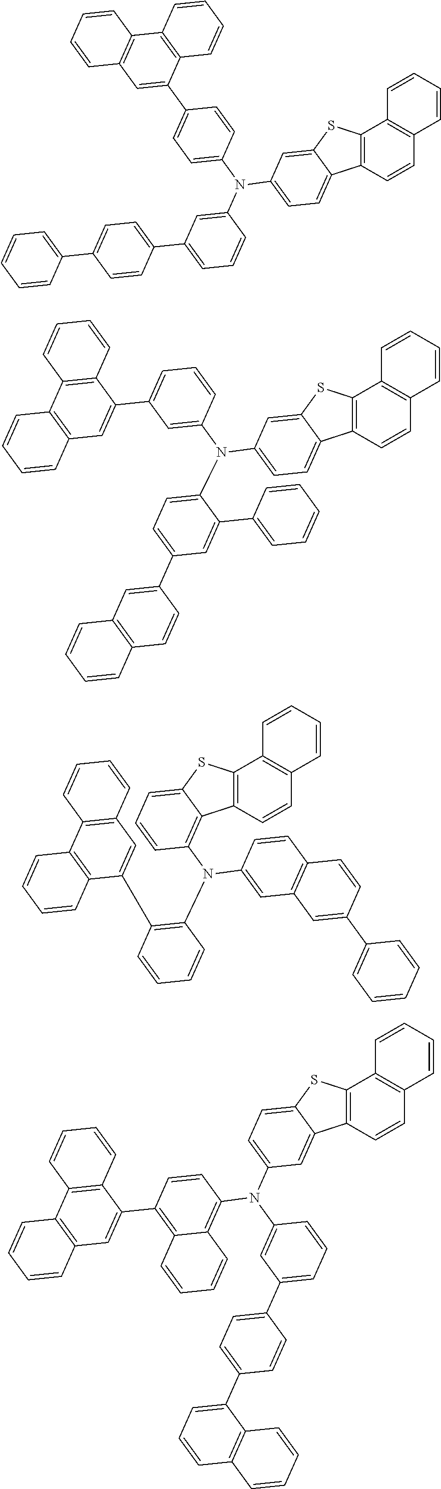

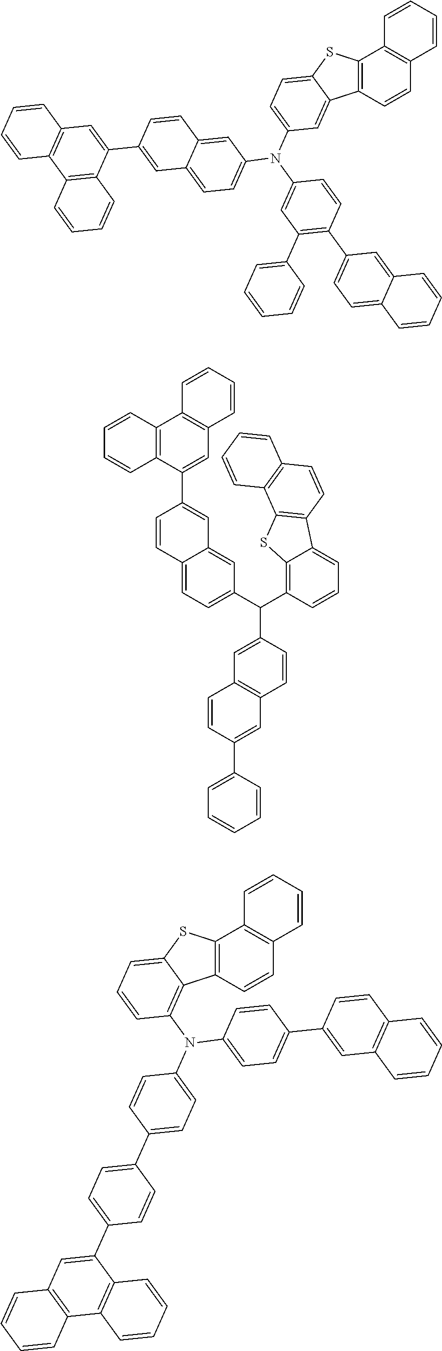 [SMILES: c1ccc(-c2cc(N(c3ccc4cc(-c5cc6ccccc6c6ccccc56)ccc4c3)c3ccc4sc5c6ccccc6ccc5c4c3)ccc2-c2ccc3ccccc3c2)cc1 ; c1ccc(-c2ccc3cc(C(c4ccc5ccc(-c6cc7ccccc7c7ccccc67)cc5c4)c4cccc5c4sc4c6ccccc6ccc54)ccc3c2)cc1 ; c1ccc2cc(-c3ccc(N(c4ccc(-c5ccc(-c6cc7ccccc7c7ccccc67)cc5)cc4)c4cccc5sc6c7ccccc7ccc6c45)cc3)ccc2c1]

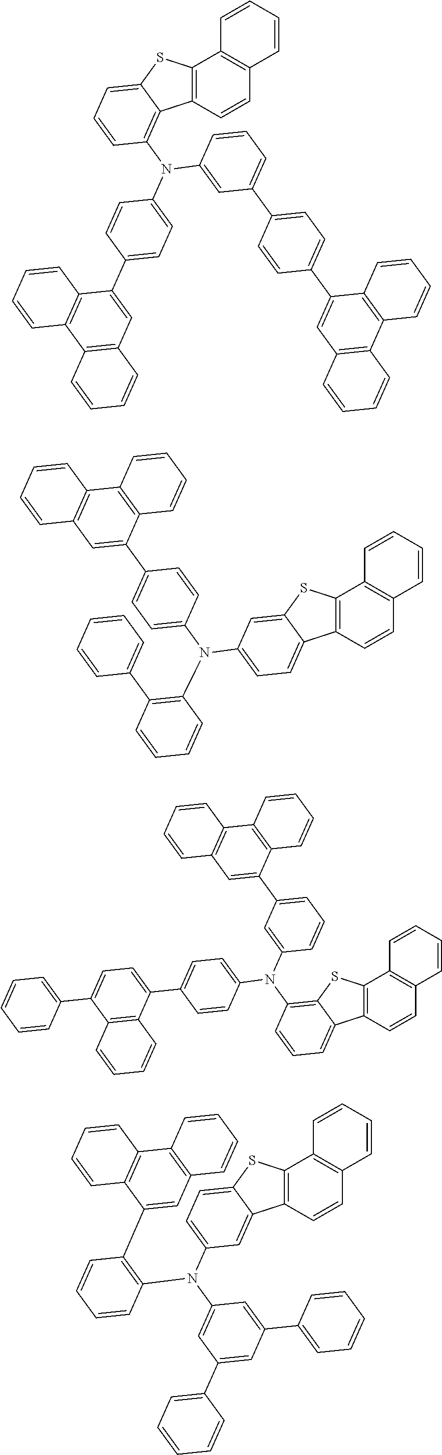 [SMILES: c1cc(-c2ccc(-c3cc4ccccc4c4ccccc34)cc2)cc(N(c2ccc(-c3cc4ccccc4c4ccccc34)cc2)c2cccc3sc4c5ccccc5ccc4c23)c1 ; c1ccc(-c2ccccc2N(c2ccc(-c3cc4ccccc4c4ccccc34)cc2)c2ccc3c(c2)sc2c4ccccc4ccc32)cc1 ; c1ccc(-c2ccc(-c3ccc(N(c4cccc(-c5cc6ccccc6c6ccccc56)c4)c4cccc5c4sc4c6ccccc6ccc54)cc3)c3ccccc23)cc1 ; c1ccc(-c2cc(-c3ccccc3)cc(N(c3ccc4sc5c6ccccc6ccc5c4c3)c3ccccc3-c3cc4ccccc4c4ccccc34)c2)cc1]

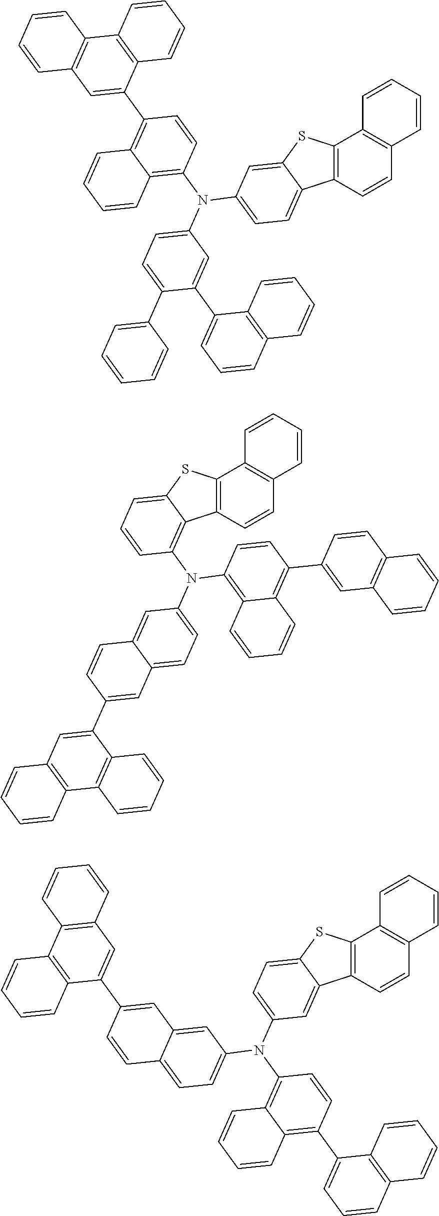 [SMILES: c1ccc(-c2ccc(N(c3ccc4c(c3)sc3c5ccccc5ccc43)c3ccc(-c4cc5ccccc5c5ccccc45)c4ccccc34)cc2-c2cccc3ccccc23)cc1 ; c1ccc2cc(-c3ccc(N(c4ccc5cc(-c6cc7ccccc7c7ccccc67)ccc5c4)c4cccc5sc6c7ccccc7ccc6c45)c4ccccc34)ccc2c1 ; c1ccc2c(-c3ccc(N(c4ccc5ccc(-c6cc7ccccc7c7ccccc67)cc5c4)c4ccc5sc6c7ccccc7ccc6c5c4)c4ccccc34)cccc2c1]

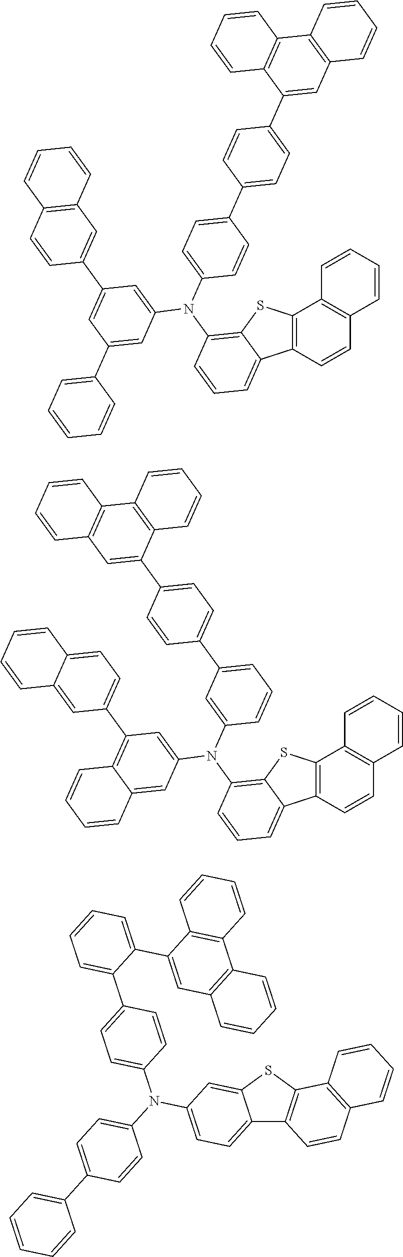

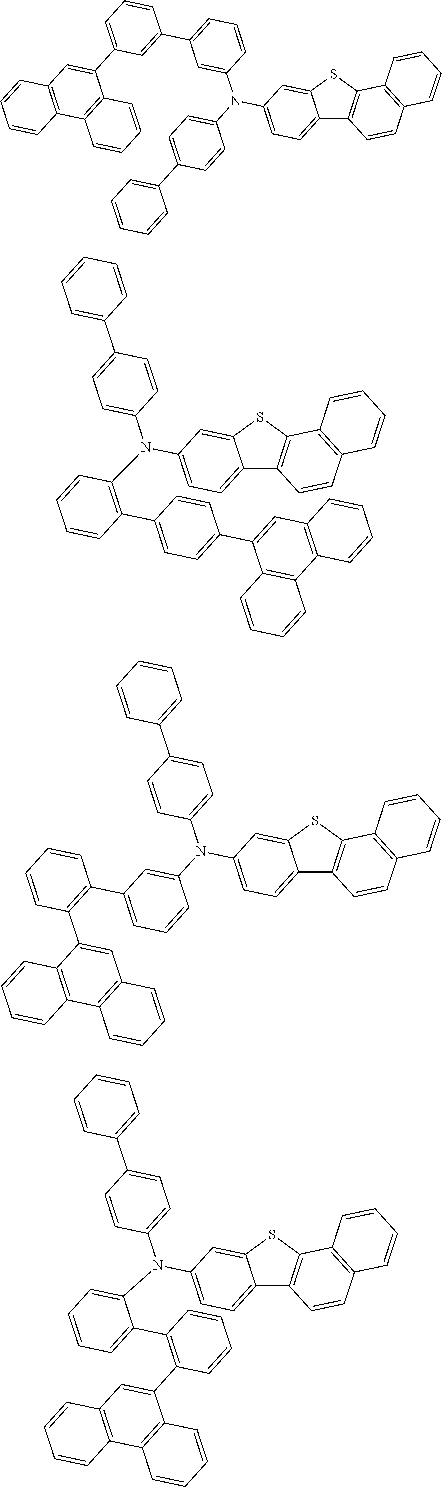

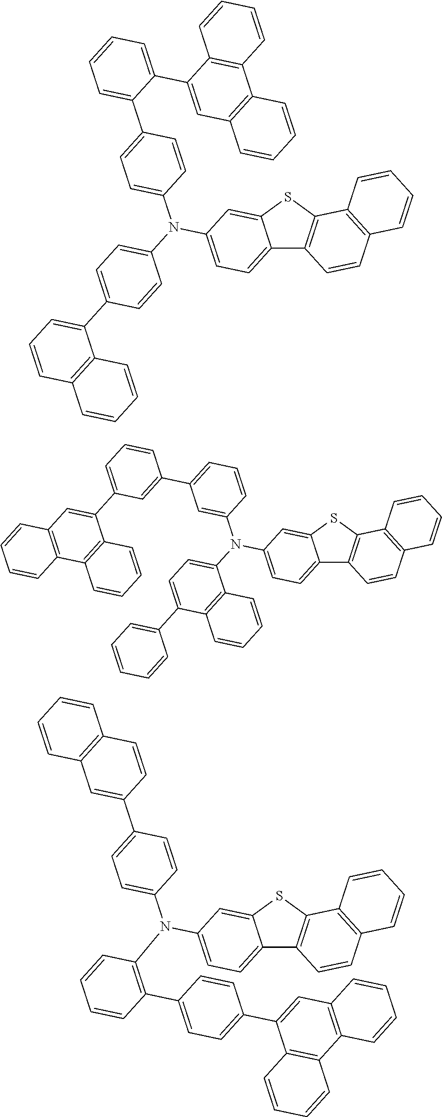

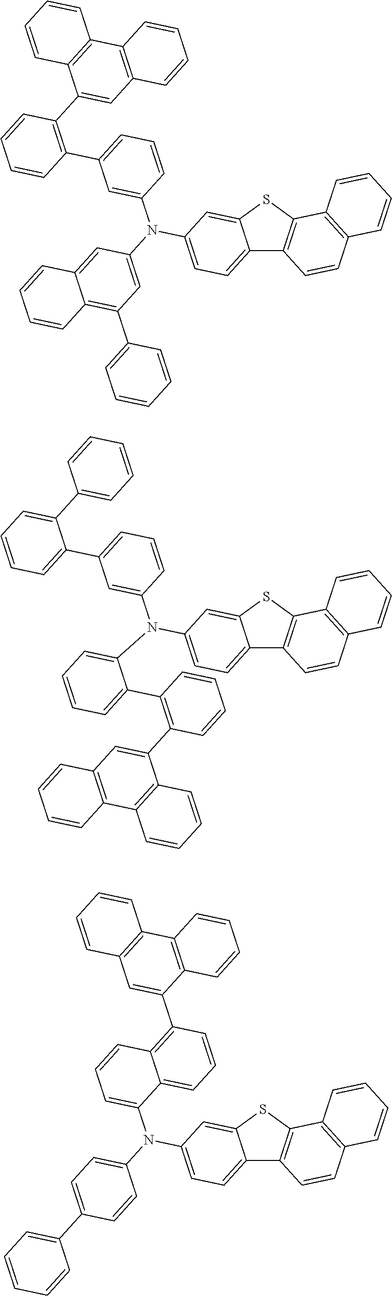

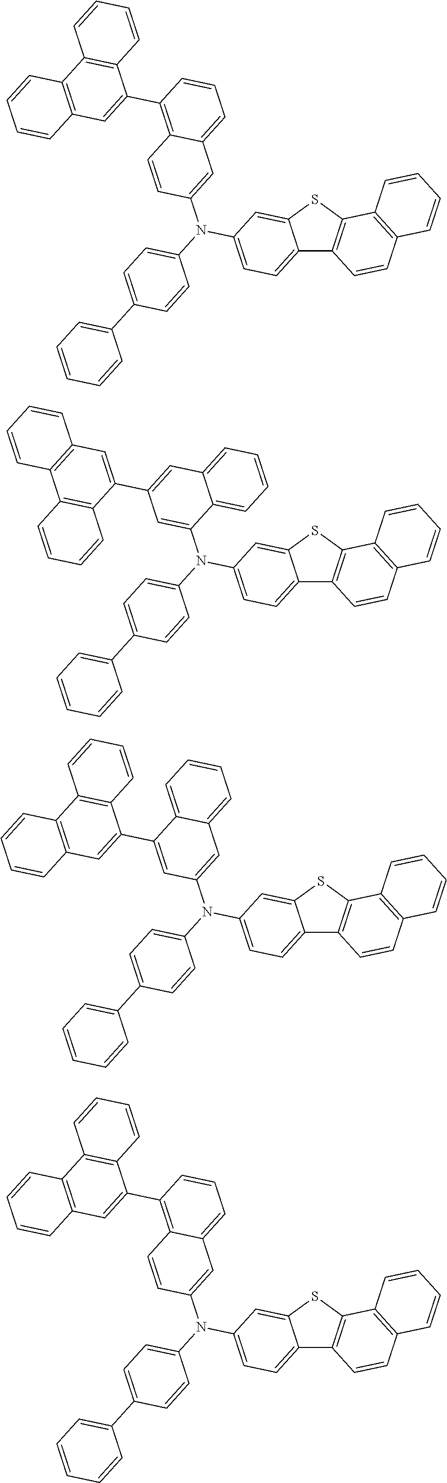

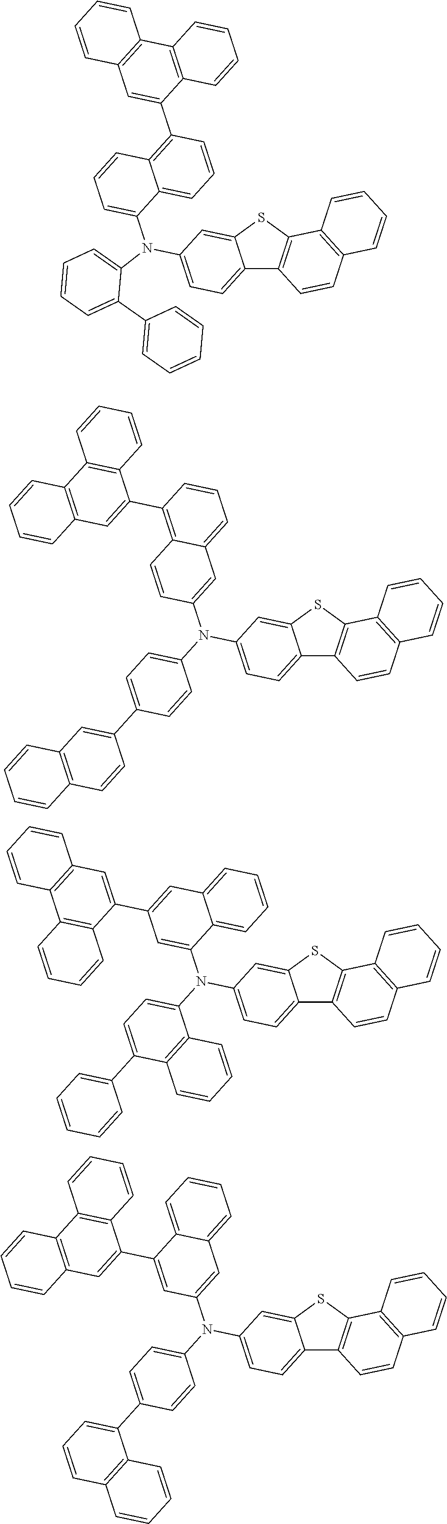

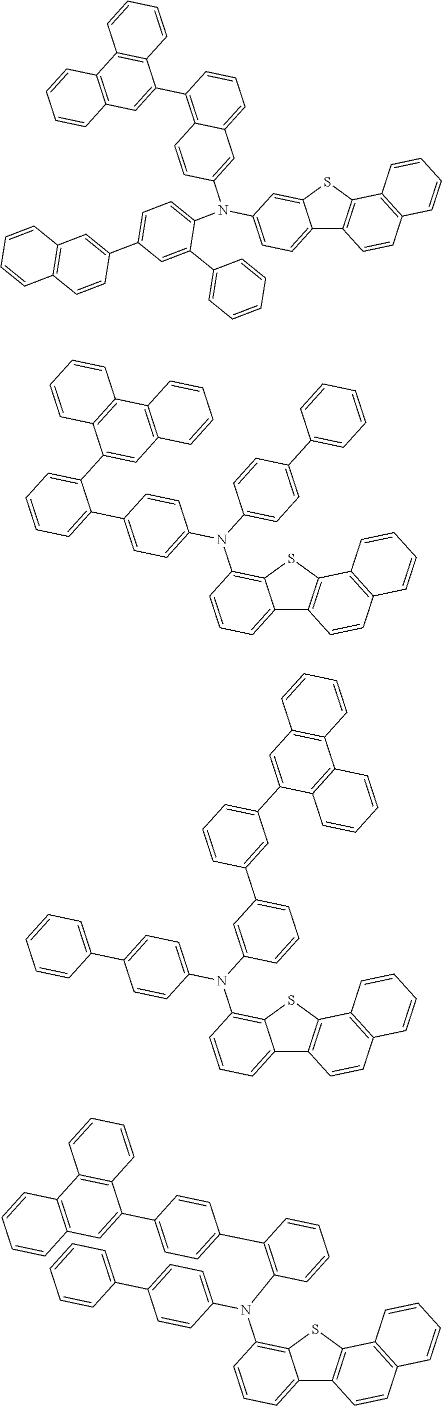

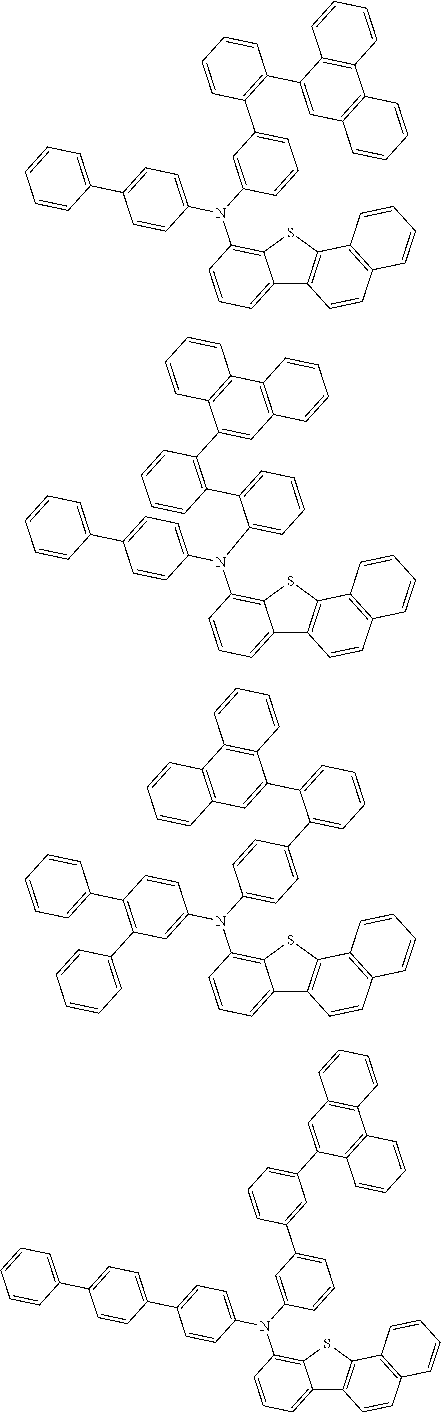

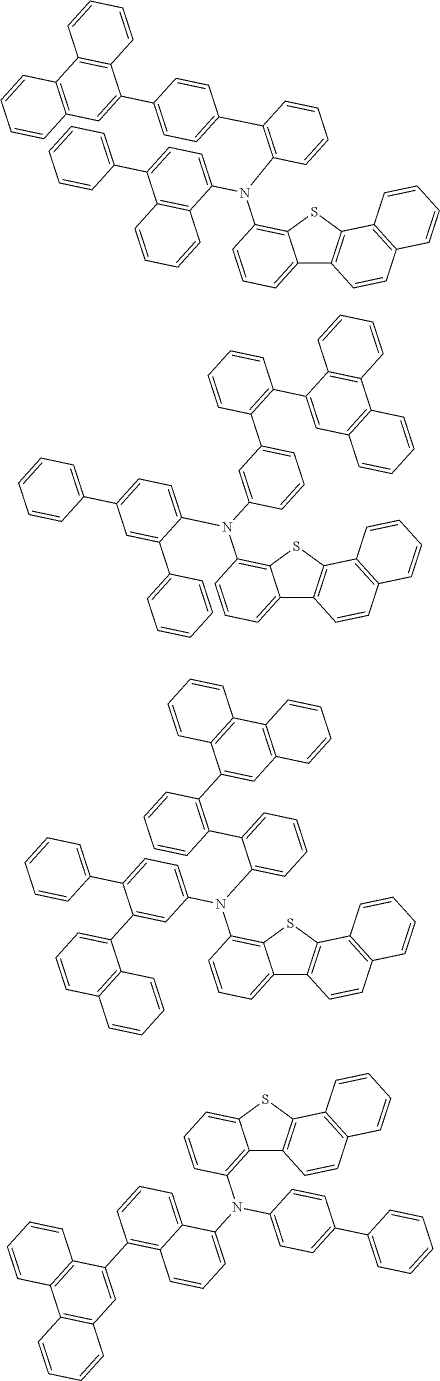

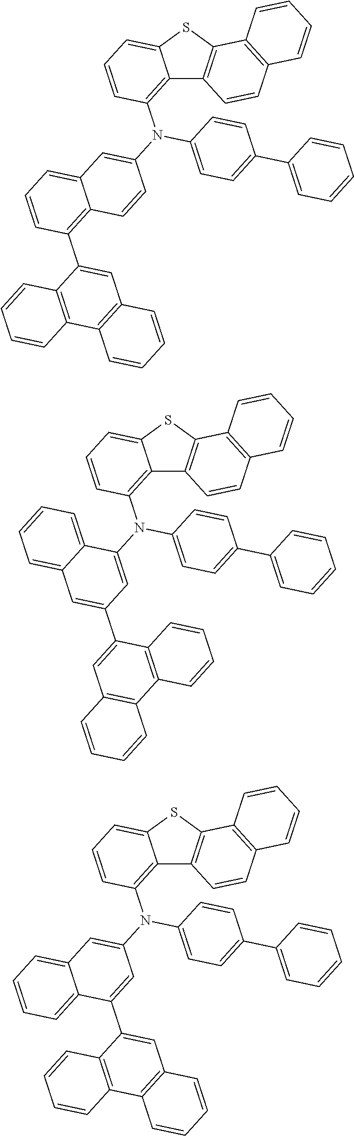 [SMILES: c1ccc(-c2ccc(N(c3ccc4c(-c5cc6ccccc6c6ccccc56)cccc4c3)c3cccc4sc5c6ccccc6ccc5c34)cc2)cc1 ; c1ccc(-c2ccc(N(c3cc(-c4cc5ccccc5c5ccccc45)cc4ccccc34)c3cccc4sc5c6ccccc6ccc5c34)cc2)cc1 ; c1ccc(-c2ccc(N(c3cc(-c4cc5ccccc5c5ccccc45)c4ccccc4c3)c3cccc4sc5c6ccccc6ccc5c34)cc2)cc1]

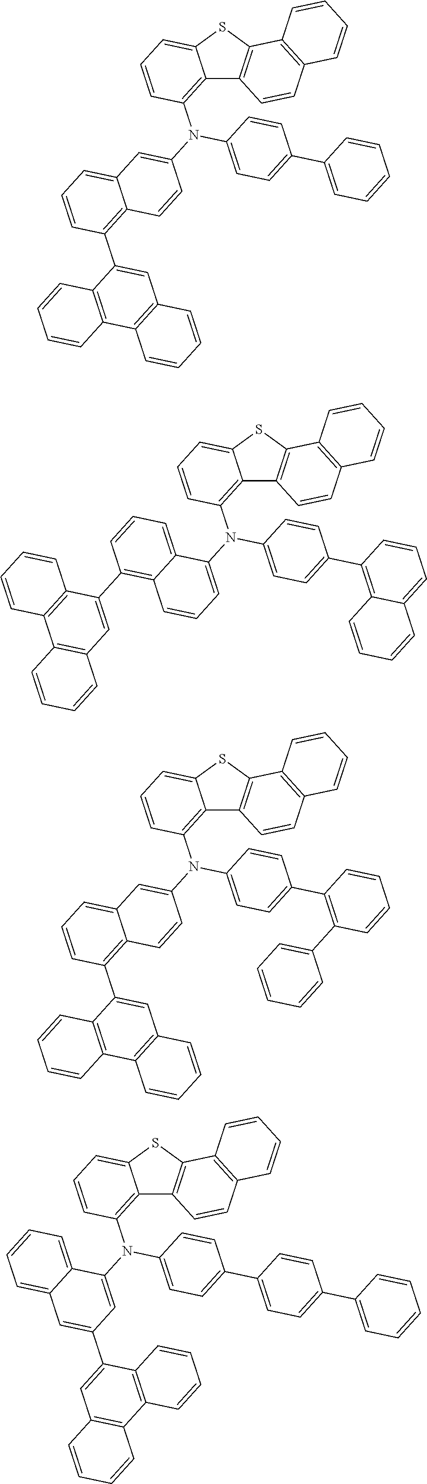 [SMILES: c1ccc(-c2ccc(N(c3ccc4c(-c5cc6ccccc6c6ccccc56)cccc4c3)c3cccc4sc5c6ccccc6ccc5c34)cc2)cc1 ; c1ccc2c(-c3ccc(N(c4cccc5c(-c6cc7ccccc7c7ccccc67)cccc45)c4cccc5sc6c7ccccc7ccc6c45)cc3)cccc2c1 ; c1ccc(-c2ccccc2-c2ccc(N(c3ccc4c(-c5cc6ccccc6c6ccccc56)cccc4c3)c3cccc4sc5c6ccccc6ccc5c34)cc2)cc1 ; c1ccc(-c2ccc(-c3ccc(N(c4cc(-c5cc6ccccc6c6ccccc56)cc5ccccc45)c4cccc5sc6c7ccccc7ccc6c45)cc3)cc2)cc1]

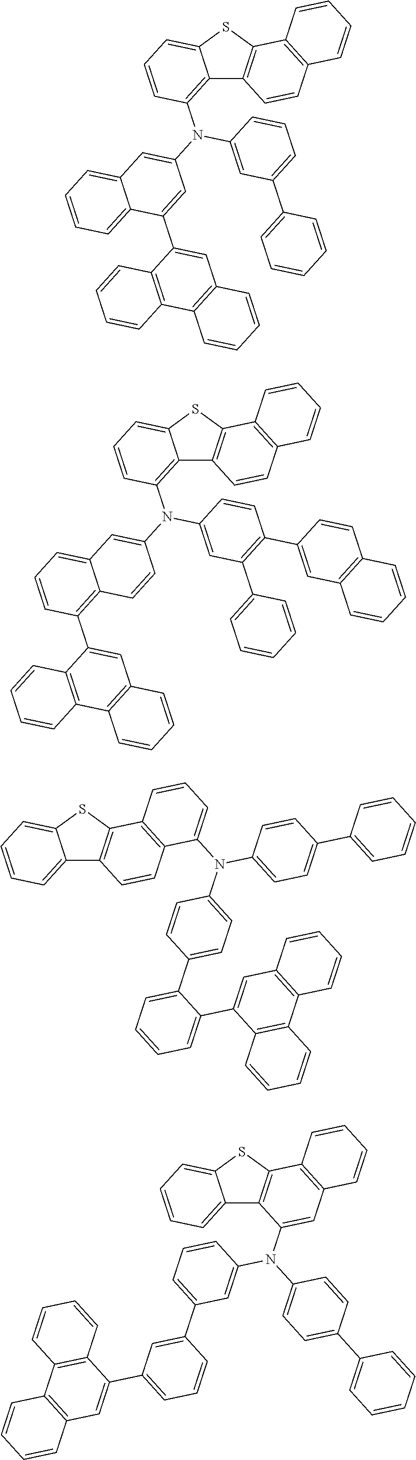

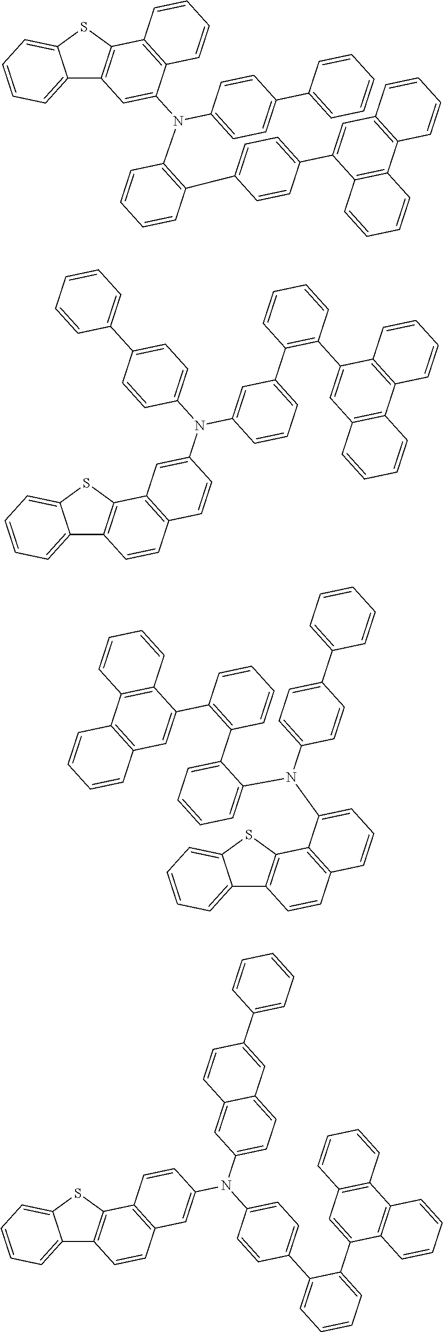

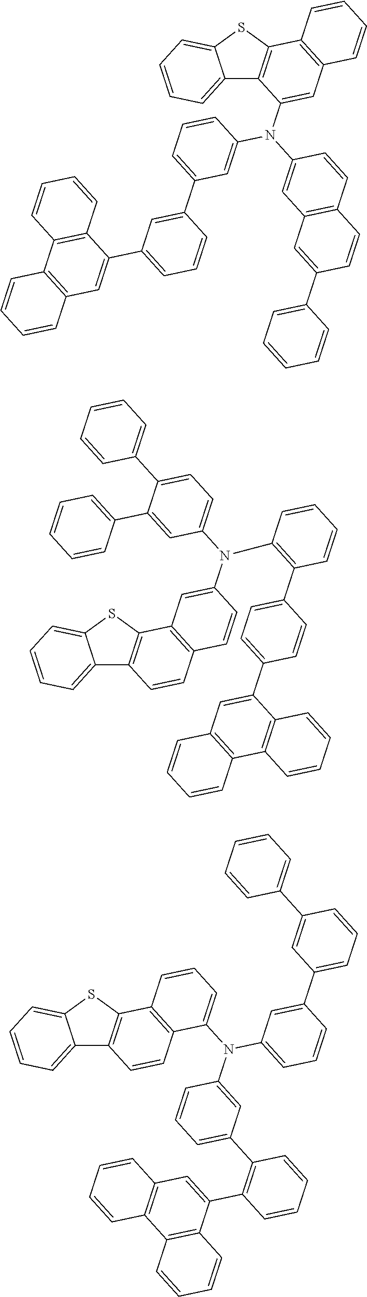

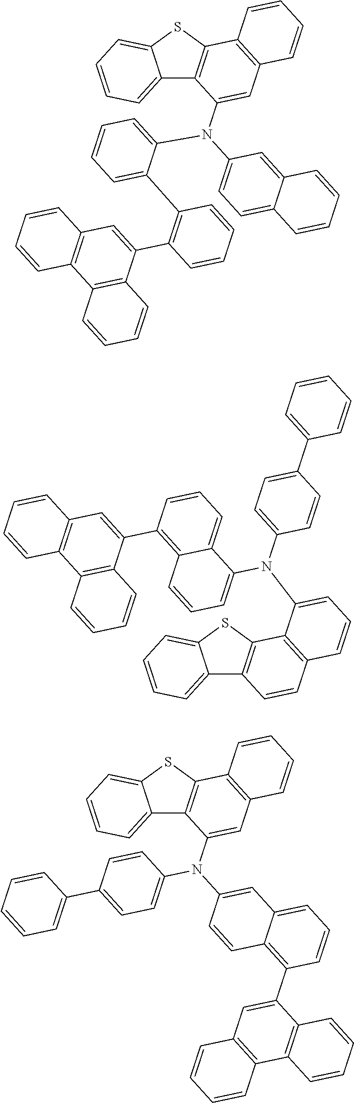

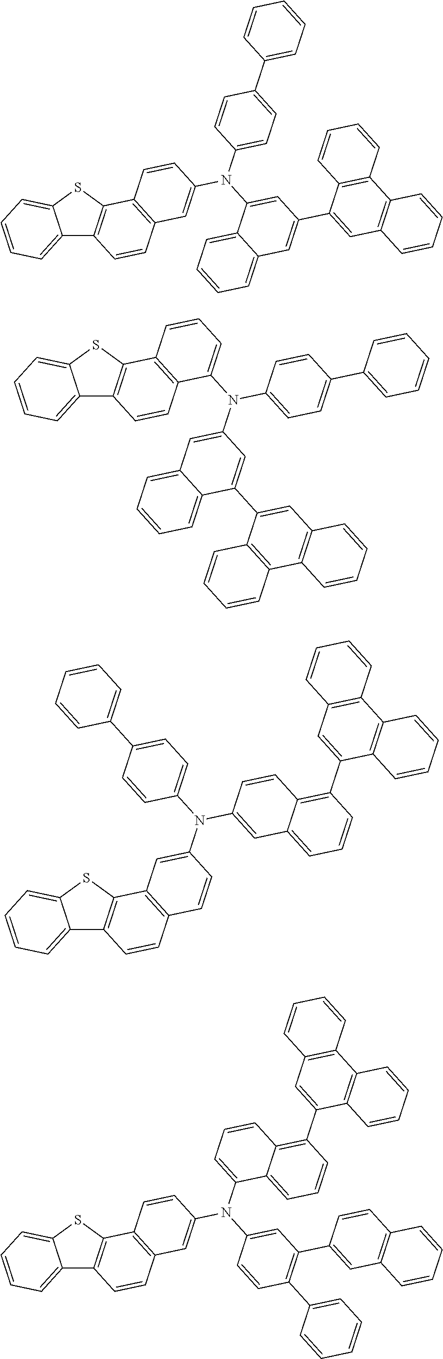

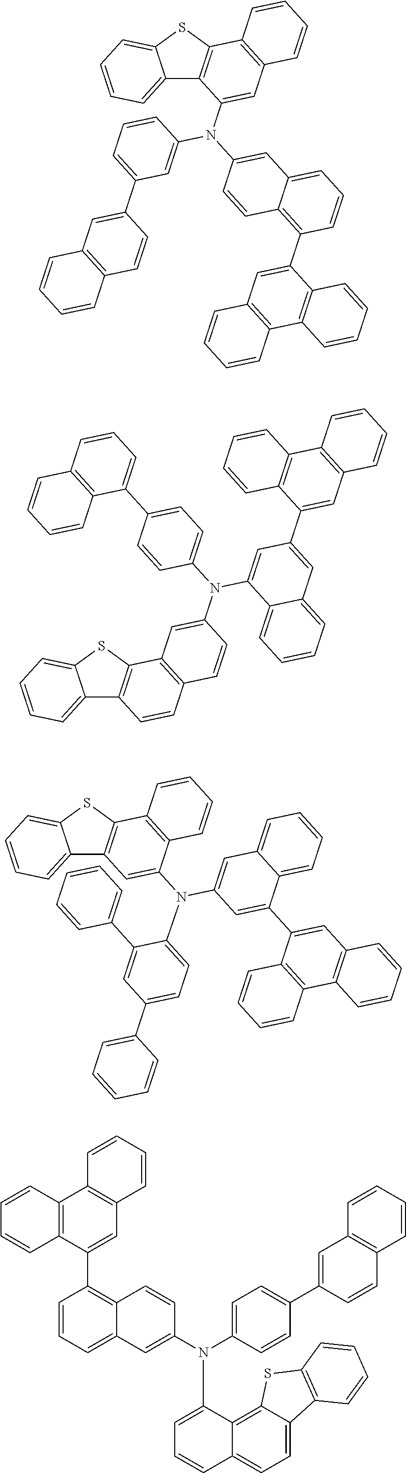

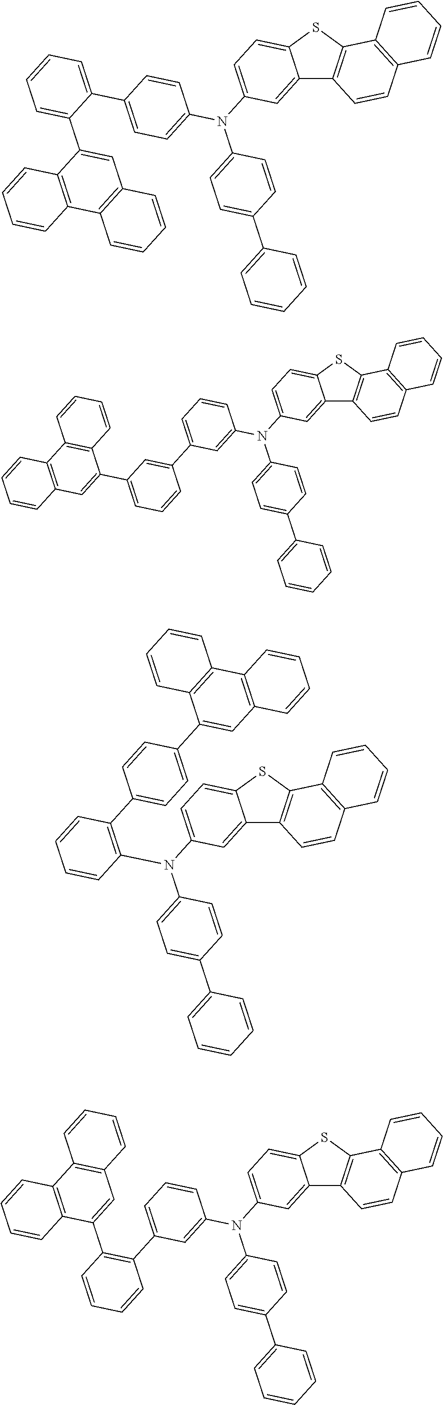 [SMILES: c1ccc(-c2ccc(N(c3ccc(-c4ccccc4-c4cc5ccccc5c5ccccc45)cc3)c3ccc4sc5c6ccccc6ccc5c4c3)cc2)cc1 ; c1ccc(-c2ccc(N(c3cccc(-c4cccc(-c5cc6ccccc6c6ccccc56)c4)c3)c3ccc4sc5c6ccccc6ccc5c4c3)cc2)cc1 ; c1ccc(-c2ccc(N(c3ccc4sc5c6ccccc6ccc5c4c3)c3ccccc3-c3ccc(-c4cc5ccccc5c5ccccc45)cc3)cc2)cc1 ; c1ccc(-c2ccc(N(c3cccc(-c4ccccc4-c4cc5ccccc5c5ccccc45)c3)c3ccc4sc5c6ccccc6ccc5c4c3)cc2)cc1]

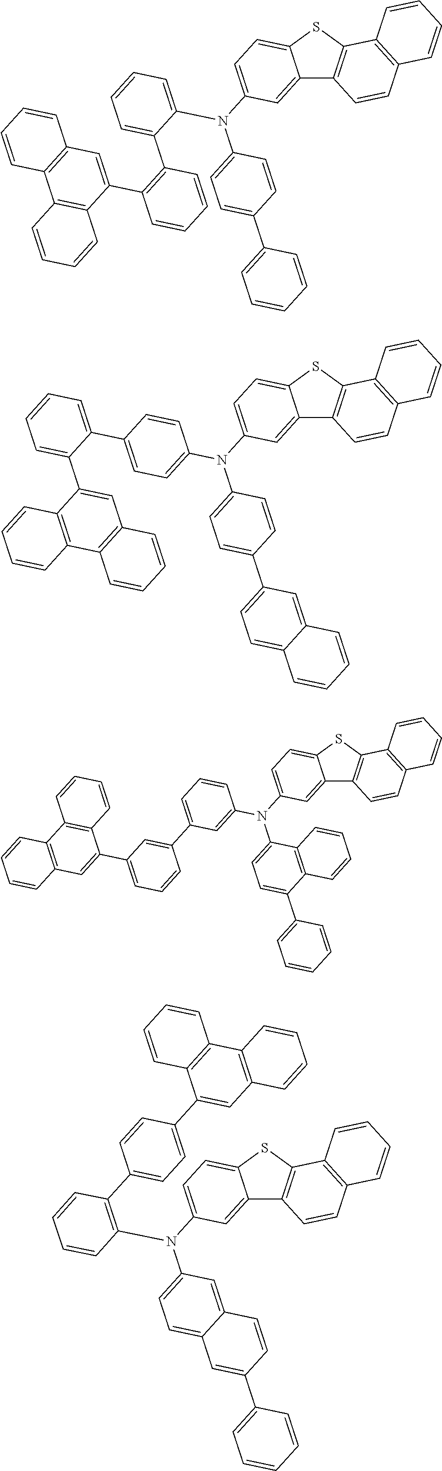 [SMILES: c1ccc(-c2ccc(N(c3ccc4sc5c6ccccc6ccc5c4c3)c3ccccc3-c3ccccc3-c3cc4ccccc4c4ccccc34)cc2)cc1 ; c1ccc(-c2cc3ccccc3c3ccccc23)c(-c2ccc(N(c3ccc(-c4ccc5ccccc5c4)cc3)c3ccc4sc5c6ccccc6ccc5c4c3)cc2)c1 ; c1ccc(-c2ccc(N(c3cccc(-c4cccc(-c5cc6ccccc6c6ccccc56)c4)c3)c3ccc4sc5c6ccccc6ccc5c4c3)c3ccccc23)cc1 ; c1ccc(-c2ccc3cc(N(c4ccc5sc6c7ccccc7ccc6c5c4)c4ccccc4-c4ccc(-c5cc6ccccc6c6ccccc56)cc4)ccc3c2)cc1]

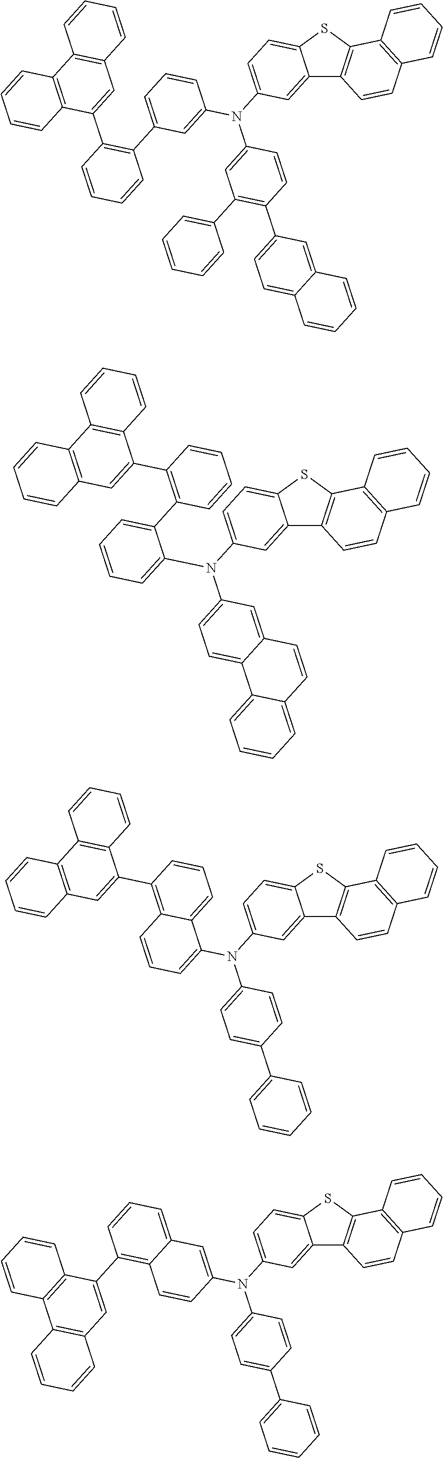 [SMILES: c1ccc(-c2cc(N(c3cccc(-c4ccccc4-c4cc5ccccc5c5ccccc45)c3)c3ccc4sc5c6ccccc6ccc5c4c3)ccc2-c2ccc3ccccc3c2)cc1 ; c1ccc(-c2cc3ccccc3c3ccccc23)c(-c2ccccc2N(c2ccc3c(ccc4ccccc43)c2)c2ccc3sc4c5ccccc5ccc4c3c2)c1 ; c1ccc(-c2ccc(N(c3ccc4sc5c6ccccc6ccc5c4c3)c3cccc4c(-c5cc6ccccc6c6ccccc56)cccc34)cc2)cc1 ; c1ccc(-c2ccc(N(c3ccc4c(-c5cc6ccccc6c6ccccc56)cccc4c3)c3ccc4sc5c6ccccc6ccc5c4c3)cc2)cc1]

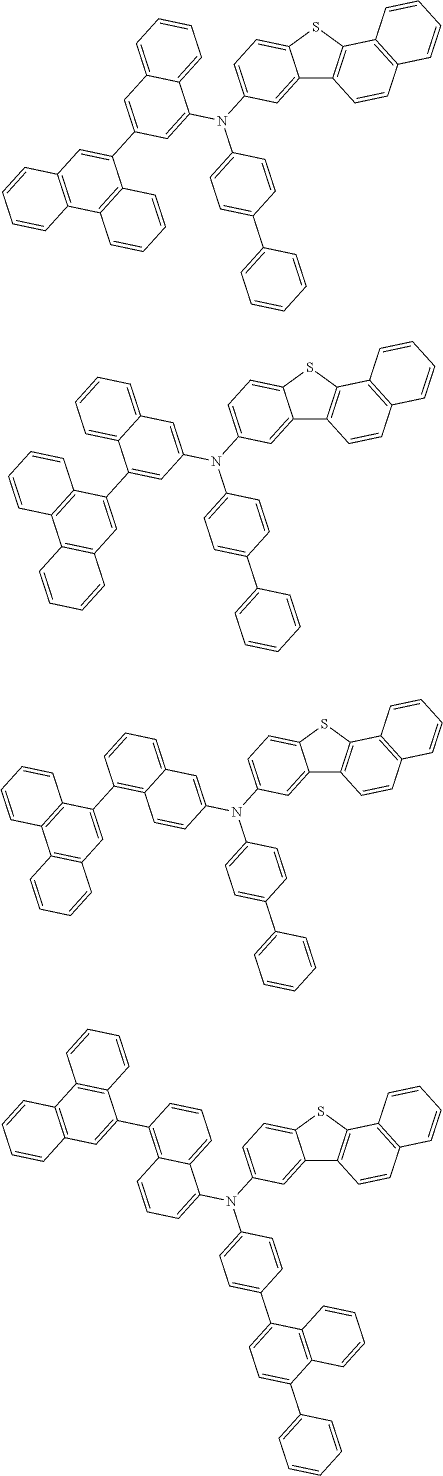 [SMILES: c1ccc(-c2ccc(N(c3ccc4sc5c6ccccc6ccc5c4c3)c3cc(-c4cc5ccccc5c5ccccc45)cc4ccccc34)cc2)cc1 ; c1ccc(-c2ccc(N(c3cc(-c4cc5ccccc5c5ccccc45)c4ccccc4c3)c3ccc4sc5c6ccccc6ccc5c4c3)cc2)cc1 ; c1ccc(-c2ccc(N(c3ccc4c(-c5cc6ccccc6c6ccccc56)cccc4c3)c3ccc4sc5c6ccccc6ccc5c4c3)cc2)cc1 ; c1ccc(-c2ccc(-c3ccc(N(c4ccc5sc6c7ccccc7ccc6c5c4)c4cccc5c(-c6cc7ccccc7c7ccccc67)cccc45)cc3)c3ccccc23)cc1]

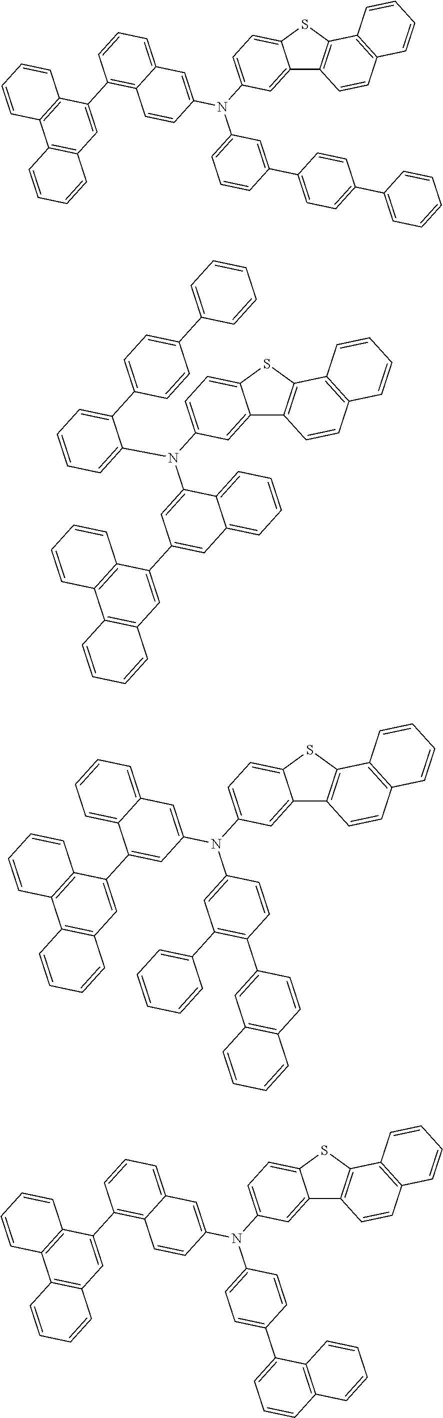 [SMILES: c1ccc(-c2ccc(-c3cccc(N(c4ccc5c(-c6cc7ccccc7c7ccccc67)cccc5c4)c4ccc5sc6c7ccccc7ccc6c5c4)c3)cc2)cc1 ; c1ccc(-c2ccc(-c3ccccc3N(c3ccc4sc5c6ccccc6ccc5c4c3)c3cc(-c4cc5ccccc5c5ccccc45)cc4ccccc34)cc2)cc1 ; c1ccc(-c2cc(N(c3cc(-c4cc5ccccc5c5ccccc45)c4ccccc4c3)c3ccc4sc5c6ccccc6ccc5c4c3)ccc2-c2ccc3ccccc3c2)cc1 ; c1ccc2c(-c3ccc(N(c4ccc5c(-c6cc7ccccc7c7ccccc67)cccc5c4)c4ccc5sc6c7ccccc7ccc6c5c4)cc3)cccc2c1]

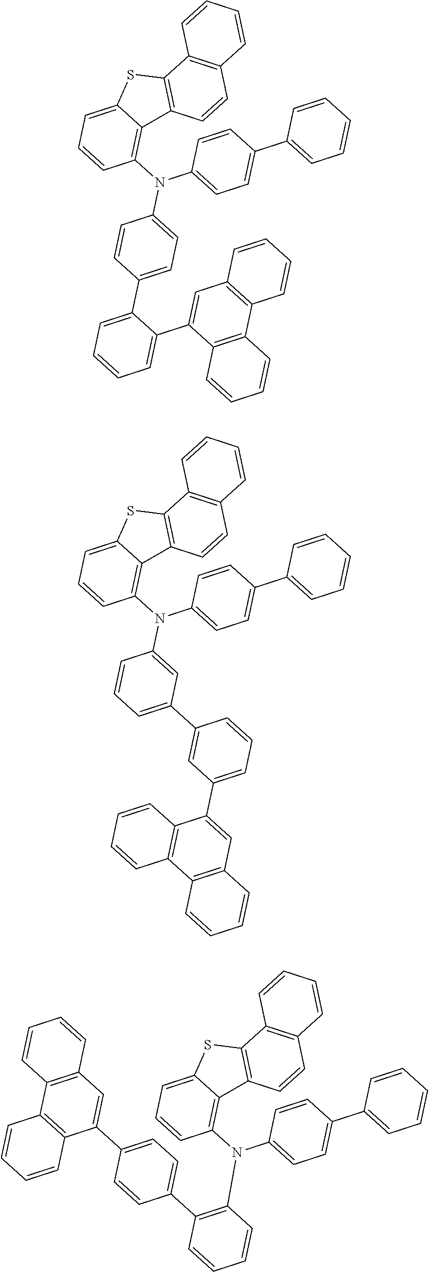

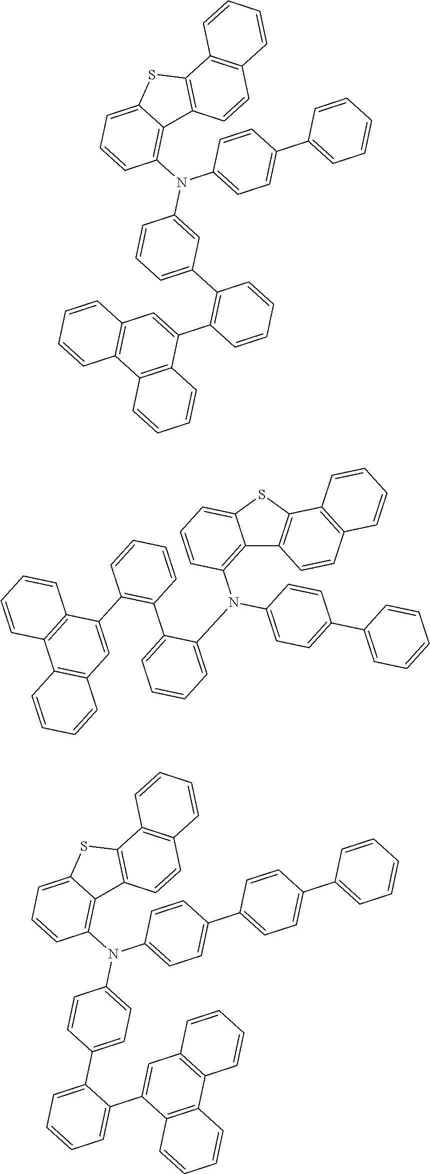 [SMILES: c1ccc(-c2ccc(N(c3cccc(-c4ccccc4-c4cc5ccccc5c5ccccc45)c3)c3cccc4sc5c6ccccc6ccc5c34)cc2)cc1 ; c1ccc(-c2ccc(N(c3ccccc3-c3ccccc3-c3cc4ccccc4c4ccccc34)c3cccc4sc5c6ccccc6ccc5c34)cc2)cc1 ; c1ccc(-c2ccc(-c3ccc(N(c4ccc(-c5ccccc5-c5cc6ccccc6c6ccccc56)cc4)c4cccc5sc6c7ccccc7ccc6c45)cc3)cc2)cc1]

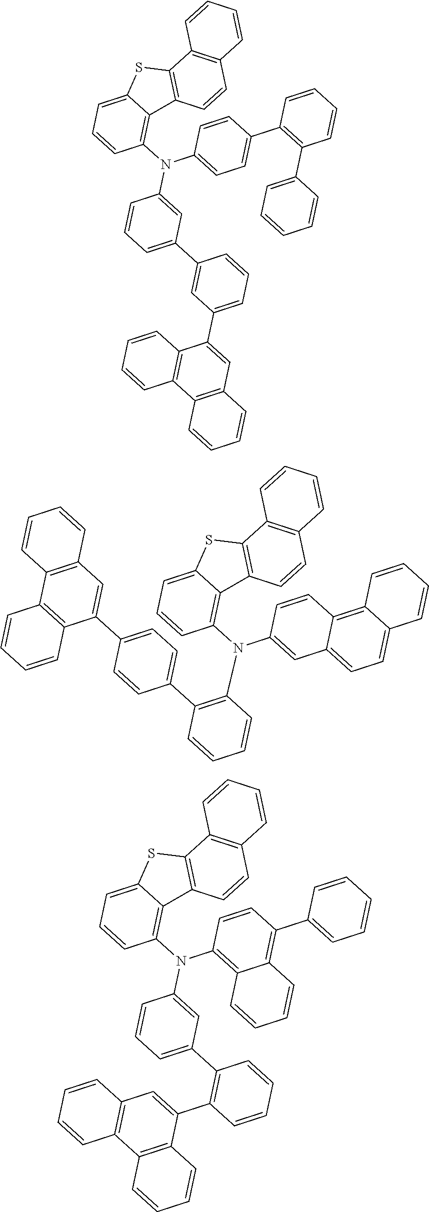

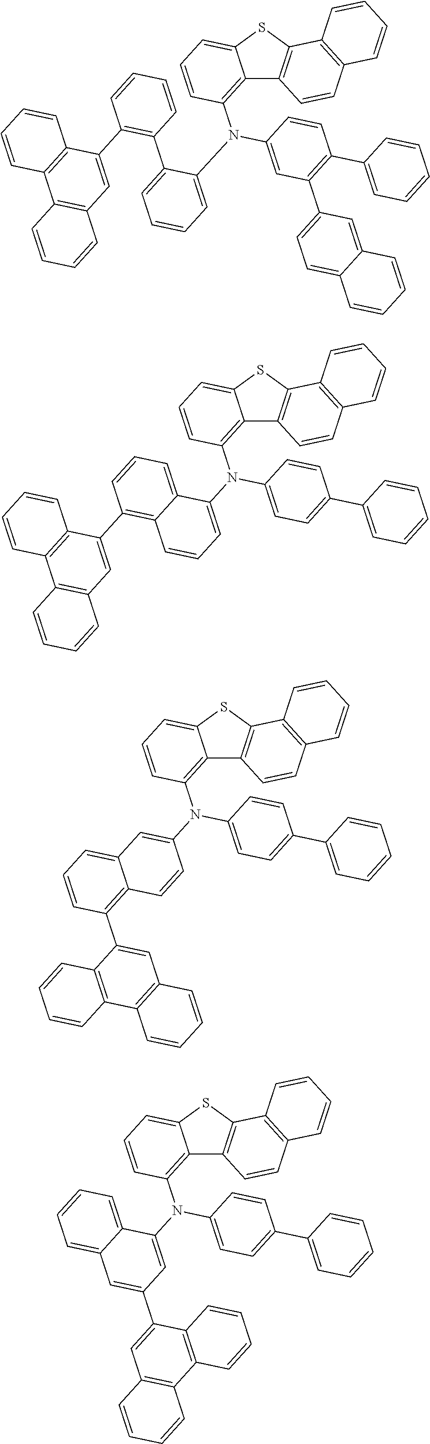

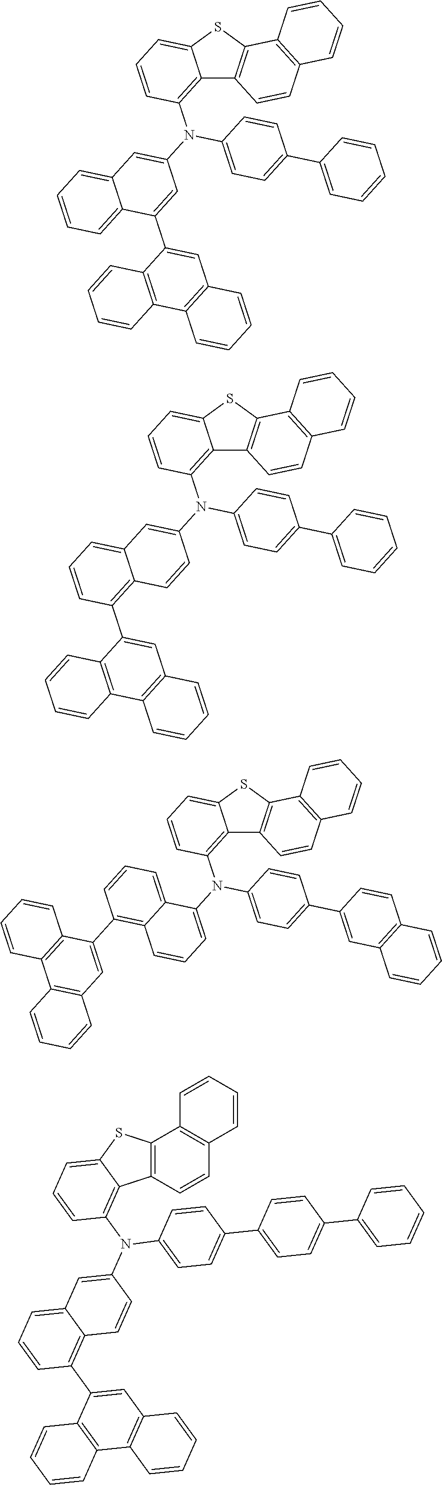 [SMILES: c1ccc(-c2ccc(N(c3cc(-c4cc5ccccc5c5ccccc45)c4ccccc4c3)c3cccc4sc5c6ccccc6ccc5c34)cc2)cc1 ; c1ccc(-c2ccc(N(c3ccc4c(-c5cc6ccccc6c6ccccc56)cccc4c3)c3cccc4sc5c6ccccc6ccc5c34)cc2)cc1 ; c1ccc2cc(-c3ccc(N(c4cccc5c(-c6cc7ccccc7c7ccccc67)cccc45)c4cccc5sc6c7ccccc7ccc6c45)cc3)ccc2c1 ; c1ccc(-c2ccc(-c3ccc(N(c4ccc5c(-c6cc7ccccc7c7ccccc67)cccc5c4)c4cccc5sc6c7ccccc7ccc6c45)cc3)cc2)cc1]

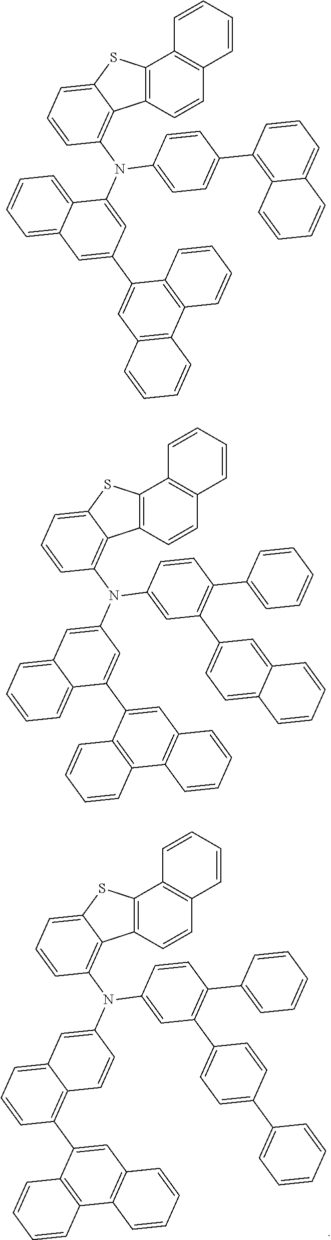

Meanwhile, the present disclosure provides a method for preparing the compound of Chemical Formula 1 as shown in the following Reaction Scheme 1 as an example.

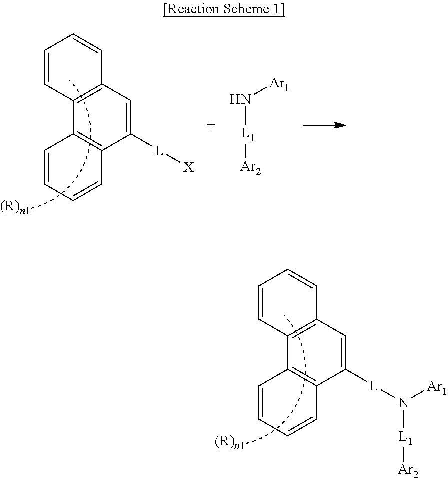

In Reaction Scheme 1, the definition of the remaining substituents except for X are the same as defined above, and X is halogen, more preferably chloro or bromo. The above reaction is an amine substitution reaction, which is preferably carried out in the presence of a palladium catalyst and a base, and a reactive group for the amine substitution reaction can be modified as known in the art. The above preparation method can be further embodied in Preparation Examples described hereinafter.

Further, the present disclosure provides an organic light emitting device comprising a compound of Chemical Formula 1. In one example, the present disclosure provides an organic light emitting device comprising: a first electrode; a second electrode that is provided opposite to the first electrode; and one or more organic material layers that are provided between the first electrode and the second electrode, wherein one or more layers of the organic material layers includes the compound of Chemical Formula 1.

The organic material layer of the organic light emitting device of the present disclosure can have a single-layer structure, or it can have a multilayered structure in which two or more organic material layers are stacked. For example, the organic light emitting device of the present disclosure can have a structure comprising a hole injection layer, a hole transport layer, a light emitting layer, an electron transport layer, an electron injection layer and the like as the organic material layer. However, the structure of the organic light emitting device is not limited thereto, and it can include a smaller number of organic layers.

Further, the organic material layer can include a light emitting layer, wherein the light emitting layer includes the compound of Chemical Formula 1. Particularly, the compound according to the present disclosure can be used as a dopant of the light emitting layer.

Further, the organic material layer can include an electron transport layer or an electron injection layer, wherein the electron transport layer or the electron injection layer includes the compound of Chemical Formula 1.

Further, the electron transport layer, the electron injection layer. or a layer for simultaneously performing electron transport and electron injection includes the compound of Chemical Formula 1.

Further, the organic material layer includes a light emitting layer or an electron transport layer, wherein the electron transport layer can include the compound of Chemical Formula 1.

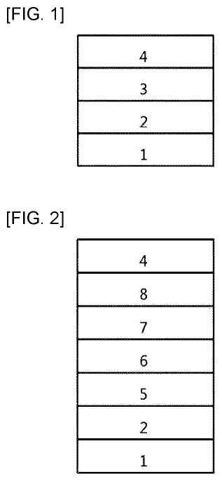

Further, the organic light emitting device according to the present disclosure can be a normal type organic light emitting device in which an anode, one or more organic material layers, and a cathode are sequentially stacked on a substrate. Further, the organic light emitting device according to the present disclosure can be an inverted type organic light emitting device in which a cathode, one or more organic material layers, and an anode are sequentially stacked on a substrate. For example, the structure of the organic light emitting device according to an embodiment of the present disclosure is illustrated in FIGS. 1 and 2.

FIG. 1 shows an example of an organic light emitting device comprising a substrate 1, an anode 2, a light emitting layer 3, and a cathode 4. In such a structure, the compound of Chemical Formula 1 can be included in the light emitting layer.

FIG. 2 shows an example of an organic light emitting device comprising a substrate 1, an anode 2, a hole injection layer 5, a hole transport layer 6, a light emitting layer 7, an electron transport layer 8, and a cathode 4. In such a structure, the compound of Chemical Formula 1 can be included in at least one layer of the hole injection layer, the hole transport layer, the light emitting layer and the electron transport layer.

The organic light emitting device according to the present disclosure can be manufactured by materials and methods known in the art, except that at least one of the organic material layers includes the compound of Chemical Formula 1. Further, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound of Chemical Formula 1 can be formed into an organic layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device can be manufactured by sequentially depositing a cathode material, an organic material layer and an anode material on a substrate (International Publication WO2003/012890). However, the manufacturing method is not limited thereto.

As an example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive compounds such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto.

The light emitting material is preferably a material which can receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex (Alq₃); a carbazole-based compound; a dimerized styryl compound; BAlq; a 10-hydroxybenzoquinoline-metal compound; a benzoxazole, benzothiazole and benzimidazole-based compound; a poly(p-phenylenevinylene)(PPV)-based polymer; a spiro compound; polyfluorene, rubrene, and the like, but are not limited thereto.

The light emitting layer can include a host material and a dopant material. The host material can be a fused aromatic ring derivative, a heterocycle-containing compound or the like. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The electron transport layer is a layer which receives electrons from an electron injection layer and transports the electrons to a light emitting layer, and an electron transport material is suitably a material which can receive electrons well from a cathode and transfer the electrons to a light emitting layer, and has a large mobility for electrons. Specific examples of the electron transport material include: an Al complex of 8-hydroxyquinoline; a complex including Alq₃; an organic radical compound; a hydroxyflavone-metal complex, and the like, but are not limited thereto. The electron transport layer can be used with any desired cathode material, as used according to the related art. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

The metal complex compound includes 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

The organic light emitting device according to the present disclosure can be a frontside emission type, a backside emission type, or a double-sided emission type based on the used material.

In addition, the compound according to the present disclosure can be included in an organic solar cell or an organic transistor in addition to an organic light emitting device.

Hereinafter, preferred examples are presented to assist in the understanding of the present disclosure. However, the following examples are only provided for a better understanding of the present disclosure, and is not intended to limit the content of the present disclosure.

### PREPARATION EXAMPLES

### Preparation Example 1

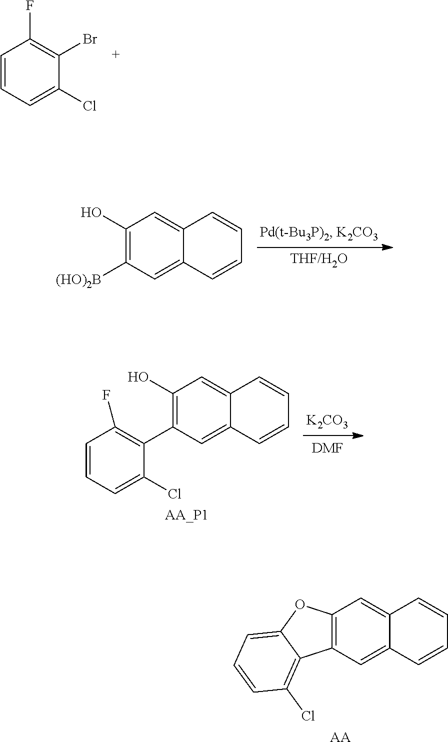

2-Bromo-1-chloro-3-fluorobenzene (15 g, 71.6 mmol) and (3-hydroxynaphthalen-2-yl)boronic acid (14.8 g, 78.8 mmol) were added to THF (300 ml) under a nitrogen atmosphere, and the mixture was stirred and refluxed. Then, potassium carbonate (29.7 g, 214.9 mmol) was dissolved in 89 ml of water and added thereto, the mixture was sufficiently stirred, and then bis(tri-tert-butylphosphine)palladium (0) (0.4 g, 0.7 mmol) was added. After reacting for 10 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, then filtered, and the filtrate was distilled under reduced pressure. The concentrated compound was purified by silica gel column chromatography to prepare 14.2 g of Compound AA_P1. (Yield: 73%, MS: [M+H]⁺=273)

Compound AA_P1 (15 g, 55 mmol) and potassium carbonate (22.8 g, 165 mmol) were added under a nitrogen atmosphere, and the mixture was stirred and refluxed. After reacting for 8 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, then filtered, and the filtrate was distilled under reduced pressure. The concentrated compound was purified by silica gel column chromatography to prepare 10.3 g of Compound AA. (Yield: 74%, MS: [M+H]⁺=253)

### Preparation Example 2

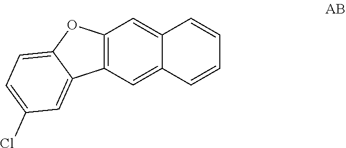 [SMILES: Clc1ccc2oc3cc4ccccc4cc3c2c1]

Compound AB was prepared in the same manner as in Preparation Example 1, except that 2-bromo-4-chloro-1-fluorobenzene was used instead of 2-bromo-1-chloro-3-fluorobenzene.

### Preparation Example 3

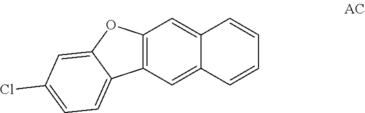 [SMILES: Clc1ccc2c(c1)oc1cc3ccccc3cc12]

Compound AC was prepared in the same manner as in Preparation Example 1, except that 1-bromo-4-chloro-2-fluorobenzene was used instead of 2-bromo-1-chloro-3-fluorobenzene.

### Preparation Example 4

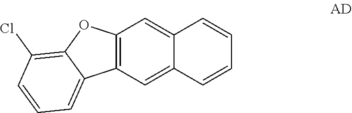 [SMILES: Clc1cccc2c1oc1cc3ccccc3cc12]

Compound AD was prepared in the same manner as in Preparation Example 1, except that 1-bromo-3-chloro-2-fluorobenzene was used instead of 2-bromo-1-chloro-3-fluorobenzene.

### Preparation Example 5

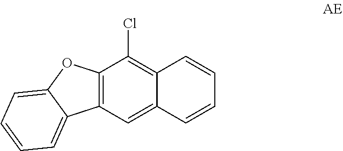 [SMILES: Clc1c2ccccc2cc2c1oc1ccccc12]

Compound AE was prepared in the same manner as in Preparation Example 1, except that 1-bromo-2-fluorobenzene was used instead of 2-bromo-1-chloro-3-fluorobenzene, and (4-chloro-3-hydroxynaphthalen-2-yl)boronic acid was used instead of (3-hydroxynaphthalen-2-yl)boronic acid.

### Preparation Example 6

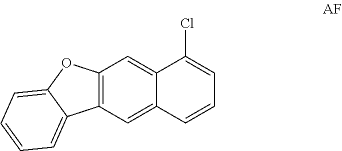 [SMILES: Clc1cccc2cc3c(cc12)oc1ccccc13]

Compound AF was prepared in the same manner as in Preparation Example 1, except that 1-bromo-2-fluorobenzene was used instead of 2-bromo-1-chloro-3-fluorobenzene, and (5-chloro-3-hydroxynaphthalen-2-yl)boronic acid was used instead of (3-hydroxynaphthalen-2-yl)boronic acid.

### Preparation Example 7

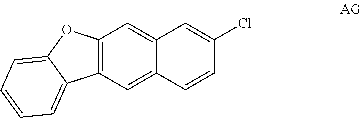 [SMILES: Clc1ccc2cc3c(cc2c1)oc1ccccc13]

Compound AG was prepared in the same manner as in Preparation Example 1, except that 1-bromo-2-fluorobenzene was used instead of 2-bromo-1-chloro-3-fluorobenzene, and (6-chloro-3-hydroxynaphthalen-2-yl)boronic acid was used instead of (3-hydroxynaphthalen-2-yl)boronic acid.

### Preparation Example 8

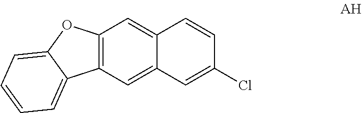 [SMILES: Clc1ccc2cc3oc4ccccc4c3cc2c1]

Compound AH was prepared in the same manner as in Preparation Example 1, except that 1-bromo-2-fluorobenzene was used instead of 2-bromo-1-chloro-3-fluorobenzene, and (7-chloro-3-hydroxynaphthalen-2-yl)boronic acid was used instead of (3-hydroxynaphthalen-2-yl)boronic acid.

### Preparation Example 9

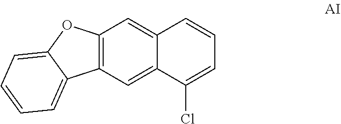 [SMILES: Clc1cccc2cc3oc4ccccc4c3cc12]

Compound AI was prepared in the same manner as in Preparation Example 1, except that 1-bromo-2-fluorobenzene was used instead of 2-bromo-1-chloro-3-fluorobenzene, and (8-chloro-3-hydroxynaphthalen-2-yl)boronic acid was used instead of (3-hydroxynaphthalen-2-yl)boronic acid.

### Preparation Example 10

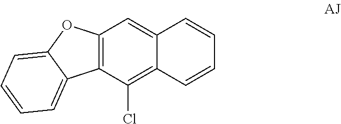 [SMILES: Clc1c2ccccc2cc2oc3ccccc3c12]

Compound AJ was prepared in the same manner as in Preparation Example 1, except that 1-bromo-2-fluorobenzene was used instead of 2-bromo-1-chloro-3-fluorobenzene, and (1-chloro-3-hydroxynaphthalen-2-yl)boronic acid was used instead of (3-hydroxynaphthalen-2-yl)boronic acid.

### Preparation Example 11

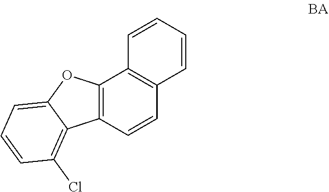 [SMILES: Clc1cccc2oc3c4ccccc4ccc3c12]

Compound BA was prepared in the same manner as in Preparation Example 1, except that (1-hydroxynaphthalen-2-yl)boronic acid was used instead of (3-hydroxynaphthalen-2-yl)boronic acid.

### Preparation Example 12

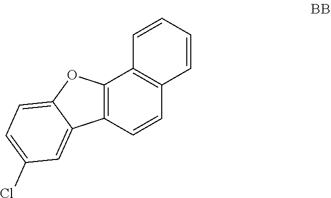 [SMILES: Clc1ccc2oc3c4ccccc4ccc3c2c1]

Compound BB was prepared in the same manner as in Preparation Example 1, except that 2-bromo-4-chloro-1-fluorobenzene was used instead of 2-bromo-1-chloro-3-fluorobenzene, and (1-hydroxynaphthalen-2-yl)boronic acid was used instead of (3-hydroxynaphthalen-2-yl)boronic acid.

### Preparation Example 13

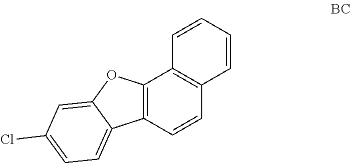 [SMILES: Clc1ccc2c(c1)oc1c3ccccc3ccc21]

Compound BC was prepared in the same manner as in Preparation Example 1, except that 1-bromo-4-chloro-2-fluorobenzene was used instead of 2-bromo-1-chloro-3-fluorobenzene, and (1-hydroxynaphthalen-2-yl)boronic acid was used instead of (3-hydroxynaphthalen-2-yl)boronic acid.

### Preparation Example 14

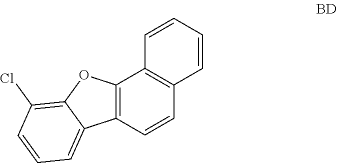 [SMILES: Clc1cccc2c1oc1c3ccccc3ccc21]

Compound BD was prepared in the same manner as in Preparation Example 1, except that 1-bromo-3-chloro-2-fluorobenzene was used instead of 2-bromo-1-chloro-3-fluorobenzene, and (1-hydroxynaphthalen-2-yl)boronic acid was used instead of (3-hydroxynaphthalen-2-yl)boronic acid.

### Preparation Example 15

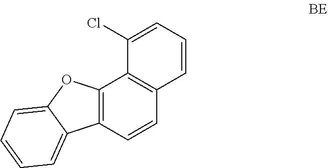 [SMILES: Clc1cccc2ccc3c4ccccc4oc3c12]

Compound BE was prepared in the same manner as in Preparation Example 1, except that 1-bromo-2-fluorobenzene was used instead of 2-bromo-1-chloro-3-fluorobenzene, and (8-chloro-1-hydroxynaphthalen-2-yl)boronic acid was used instead of (3-hydroxynaphthalen-2-yl)boronic acid.

### Preparation Example 16

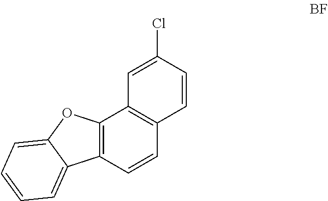 [SMILES: Clc1ccc2ccc3c4ccccc4oc3c2c1]

Compound BF was prepared in the same manner as in Preparation Example 1, except that 1-bromo-2-fluorobenzene was used instead of 2-bromo-1-chloro-3-fluorobenzene, and (7-chloro-1-hydroxynaphthalen-2-yl)boronic acid was used instead of (3-hydroxynaphthalen-2-yl)boronic acid.

### Preparation Example 17

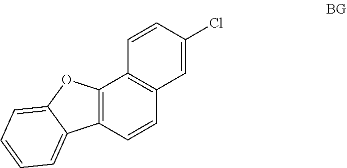 [SMILES: Clc1ccc2c(ccc3c4ccccc4oc23)c1]

Compound BG was prepared in the same manner as in Preparation Example 1, except that 1-bromo-2-fluorobenzene was used instead of 2-bromo-1-chloro-3-fluorobenzene, and (6-chloro-1-hydroxynaphthalen-2-yl)boronic acid was used instead of (3-hydroxynaphthalen-2-yl)boronic acid.

### Preparation Example 18

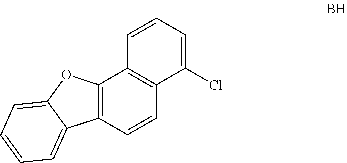 [SMILES: Clc1cccc2c1ccc1c3ccccc3oc21]

Compound BH was prepared in the same manner as in Preparation Example 1, except that 1-bromo-2-fluorobenzene was used instead of 2-bromo-1-chloro-3-fluorobenzene, and (5-chloro-1-hydroxynaphthalen-2-yl)boronic acid was used instead of (3-hydroxynaphthalen-2-yl)boronic acid.

### Preparation Example 19

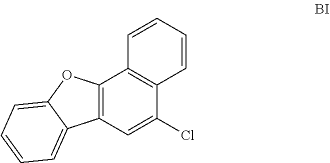 [SMILES: Clc1cc2c3ccccc3oc2c2ccccc12]

Compound BI was prepared in the same manner as in Preparation Example 1, except that 1-bromo-2-fluorobenzene was used instead of 2-bromo-1-chloro-3-fluorobenzene, and (4-chloro-1-hydroxynaphthalen-2-yl)boronic acid was used instead of (3-hydroxynaphthalen-2-yl)boronic acid.

### Preparation Example 20

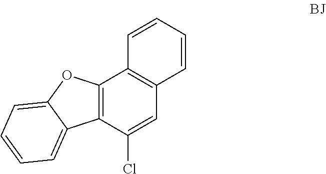 [SMILES: Clc1cc2ccccc2c2oc3ccccc3c12]

Compound BJ was prepared in the same manner as in Preparation Example 1, except that 1-bromo-2-fluorobenzene was used instead of 2-bromo-1-chloro-3-fluorobenzene, and (3-chloro-1-hydroxynaphthalen-2-yl)boronic acid was used instead of (3-hydroxynaphthalen-2-yl)boronic acid.

### Preparation Example 21

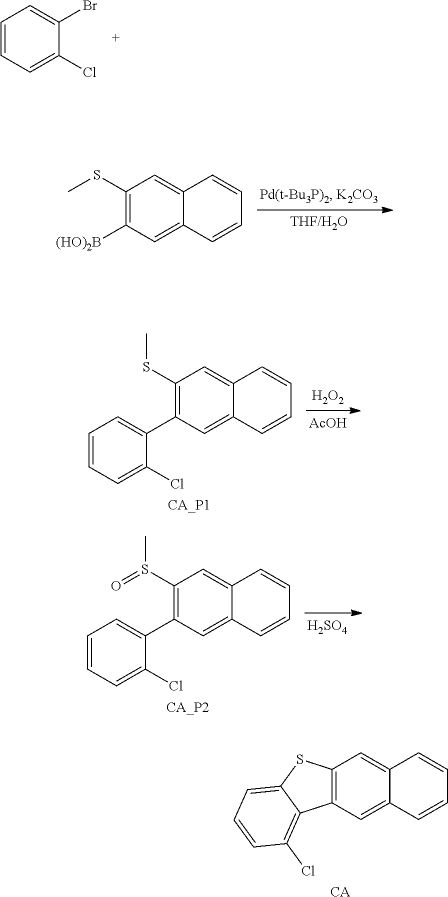

1-Bromo-2-chlorobenzene (15 g, 78.3 mmol) and (3-(methylthio)-naphthalen-2-yl)boronic acid (18.8 g, 86.2 mmol) were added to THF (300 ml) under a nitrogen atmosphere, and the mixture was stirred and refluxed. Then, potassium carbonate (32.5 g, 235 mmol) was dissolved in 97 ml of water and added thereto, the mixture was sufficiently stirred, and then bis(tri-tert-butylphosphine)palladium (0) (0.4 g, 0.8 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 16.2 g of Compound CA_P1. (Yield: 73%, MS: [M+H]⁺=285)

Compound CA_P1 (15 g, 52.7 mmol) and hydrogen peroxide (3.6 g, 105.3 mmol) were added to acetic acid (200 ml) under a nitrogen atmosphere, and the mixture was stirred and refluxed. After reacting for 3 hours, the reaction mixture was poured into water, and crystals were precipitated and filtered. The filtered solid was dissolved in chloroform, washed twice with water, the organic layer was separated, anhydrous magnesium sulfate was added, stirred, then filtered, and the filtrate was distilled under reduced pressure. The concentrated compound was purified by silica gel column chromatography to prepare 10.4 g of Compound CA_P2 (Yield: 66%, MS: [M+H]⁺=301)

Compound CA_P2 (15 g, 49.9 mmol) was added to H₂SO₄ (200 ml) under a nitrogen atmosphere, and the mixture was stirred and refluxed. After the reaction was completed after 2 hours, the reaction mixture was poured into water, and crystals were precipitated and filtered. The filtered solid was dissolved again in chloroform, washed twice with water, the organic layer was separated, anhydrous magnesium sulfate was added, stirred, then filtered, and the filtrate was distilled under reduced pressure. The concentrated compound was purified by silica gel column chromatography to prepare 9.2 g of Compound CA (Yield: 69%, MS: [M+H]⁺=269)

### Preparation Example 22

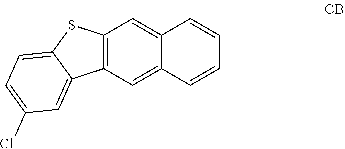 [SMILES: Clc1ccc2sc3cc4ccccc4cc3c2c1]

Compound CB was prepared in the same manner as in Preparation Example 21, except that 1-bromo-3-chlorobenzene was used instead of 1-bromo-2-chlorobenzene.

### Preparation Example 23

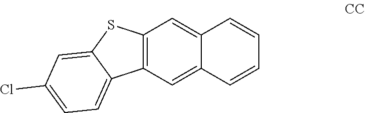 [SMILES: Clc1ccc2c(c1)sc1cc3ccccc3cc12]

Compound CC was prepared in the same manner as in Preparation Example 21, except that 1-bromo-4-chlorobenzene was used instead of 1-bromo-2-chlorobenzene.

### Preparation Example 24

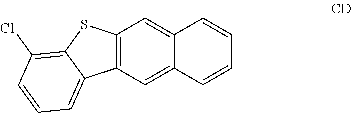 [SMILES: Clc1cccc2c1sc1cc3ccccc3cc12]

Compound CD was prepared in the same manner as in Preparation Example 21, except that 1-bromo-3-chlorobenzene was used instead of 1-bromo-2-chlorobenzene.

### Preparation Example 25

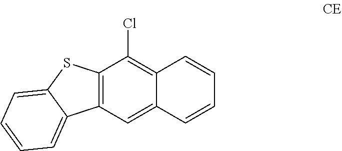 [SMILES: Clc1c2ccccc2cc2c1sc1ccccc12]

Compound CE was prepared in the same manner as in Preparation Example 21, except that bromobenzene was used instead of 1-bromo-2-chlorobenzene, and (4-chloro-3-(methylthio)naphthalen-2-yl)boronic acid was used instead of (3-(methylthio)naphthalen-2-yl)boronic acid.

### Preparation Example 26

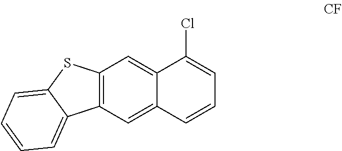 [SMILES: Clc1cccc2cc3c(cc12)sc1ccccc13]

Compound CF was prepared in the same manner as in Preparation Example 21, except that bromobenzene was used instead of 1-bromo-2-chlorobenzene, and 1-chloro-2-(methylsulfinyl)-3-phenylnaphthalene was used instead of (3-(methylthio)naphthalen-2-yl)boronic acid.

### Preparation Example 27

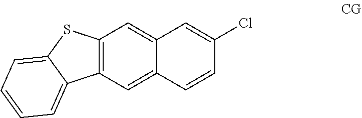 [SMILES: Clc1ccc2cc3c(cc2c1)sc1ccccc13]

Compound CG was prepared in the same manner as in Preparation Example 21, except that bromobenzene was used instead of 1-bromo-2-chlorobenzene, and (5-chloro-3-(methylthio)naphthalen-2-yl)boronic acid was used instead of (3-(methylthio)naphthalen-2-yl)boronic acid.

### Preparation Example 28

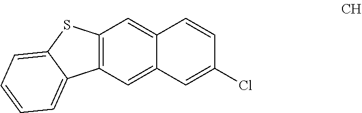 [SMILES: Clc1ccc2cc3sc4ccccc4c3cc2c1]

Compound CH was prepared in the same manner as in Preparation Example 21, except that bromobenzene was used instead of 1-bromo-2-chlorobenzene, and (6-chloro-3-(methylthio)naphthalen-2-yl)boronic acid was used instead of (3-(methylthio)naphthalen-2-yl)boronic acid.

### Preparation Example 29

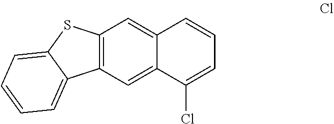 [SMILES: Clc1cccc2cc3sc4ccccc4c3cc12]

Compound CI was prepared in the same manner as in Preparation Example 21, except that bromobenzene was used instead of 1-bromo-2-chlorobenzene, and (7-chloro-3-(methylthio)naphthalen-2-yl)boronic acid was used instead of (3-(methylthio)naphthalen-2-yl)boronic acid.

### Preparation Example 30

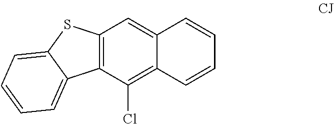 [SMILES: Clc1c2ccccc2cc2sc3ccccc3c12]

Compound CJ was prepared in the same manner as in Preparation Example 21, except that bromobenzene was used instead of 1-bromo-2-chlorobenzene, and (8-chloro-3-(methylthio)naphthalen-2-yl)boronic acid was used instead of (3-(methylthio)naphthalen-2-yl)boronic acid.

### Preparation Example 31

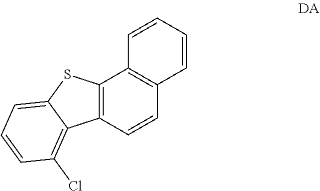 [SMILES: Clc1cccc2sc3c4ccccc4ccc3c12]

Compound DA was prepared in the same manner as in Preparation Example 21, except that (1-(methylthio)naphthalen-2-yl)boronic acid was used instead of (3-(methylthio)naphthalen-2-yl)boronic acid.

### Preparation Example 32

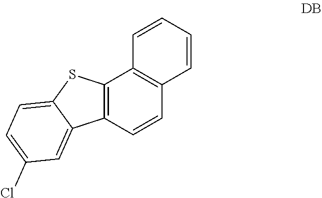 [SMILES: Clc1ccc2sc3c4ccccc4ccc3c2c1]

Compound DB was prepared in the same manner as in Preparation Example 21, except that 1-bromo-3-chlorobenzene was used instead of 1-bromo-2-chlorobenzene, and (1-(methylthio)naphthalen-2-yl)boronic acid was used instead of (3-(methylthio)naphthalen-2-yl)boronic acid.

### Preparation Example 33

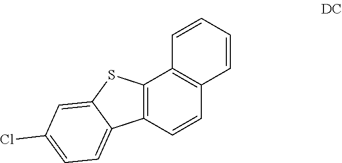 [SMILES: Clc1ccc2c(c1)sc1c3ccccc3ccc21]

Compound DC was prepared in the same manner as in Preparation Example 21, except that 1-bromo-4-chlorobenzene was used instead of 1-bromo-2-chlorobenzene, and (1-(methylthio)naphthalen-2-yl)boronic acid was used instead of (3-(methylthio)naphthalen-2-yl)boronic acid.

### Preparation Example 34

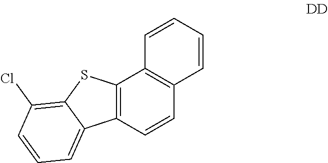 [SMILES: Clc1cccc2c1sc1c3ccccc3ccc21]

Compound DD was prepared in the same manner as in Preparation Example 21, except that 1-bromo-3-chlorobenzene was used instead of 1-bromo-2-chlorobenzene, and (1-(methylthio)naphthalen-2-yl)boronic acid was used instead of (3-(methylthio)naphthalen-2-yl)boronic acid.

### Preparation Example 35

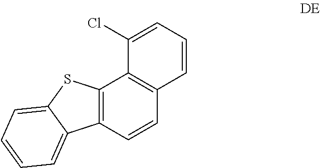 [SMILES: Clc1cccc2ccc3c4ccccc4sc3c12]

Compound DE was prepared in the same manner as in Preparation Example 21, except that bromobenzene was used instead of 1-bromo-2-chlorobenzene, and (8-chloro-1-(methylthio)naphthalen-2-yl)boronic acid was used instead of (3-(methylthio)naphthalen-2-yl)boronic acid.

### Preparation Example 36

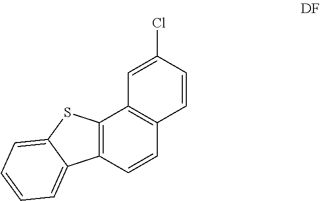 [SMILES: Clc1ccc2ccc3c4ccccc4sc3c2c1]

Compound DF was prepared in the same manner as in Preparation Example 21, except that bromobenzene was used instead of 1-bromo-2-chlorobenzene, and (7-chloro-1-(methylthio)naphthalen-2-yl)boronic acid was used instead of (3-(methylthio)naphthalen-2-yl)boronic acid.

### Preparation Example 37

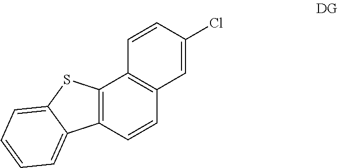 [SMILES: Clc1ccc2c(ccc3c4ccccc4sc23)c1]

Compound DG was prepared in the same manner as in Preparation Example 21, except that bromobenzene was used instead of 1-bromo-2-chlorobenzene, and (6-chloro-1-(methylthio)naphthalen-2-yl)boronic acid was used instead of (3-(methylthio)naphthalen-2-yl)boronic acid.

### Preparation Example 38

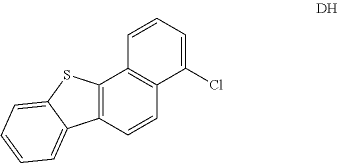 [SMILES: Clc1cccc2c1ccc1c3ccccc3sc21]

Compound DH was prepared in the same manner as in Preparation Example 21, except that bromobenzene was used instead of 1-bromo-2-chlorobenzene, and (5-chloro-1-(methylthio)naphthalen-2-yl)boronic acid was used instead of (3-(methylthio)naphthalen-2-yl)boronic acid.

### Preparation Example 39

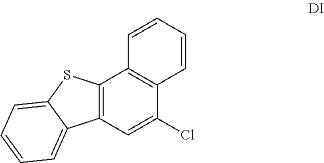 [SMILES: Clc1cc2c3ccccc3sc2c2ccccc12]

Compound DI was prepared in the same manner as in Preparation Example 21, except that bromobenzene was used instead of 1-bromo-2-chlorobenzene, and (4-chloro-1-(methylthio)naphthalen-2-yl)boronic acid was used instead of (3-(methylthio)naphthalen-2-yl)boronic acid.

### Preparation Example 40

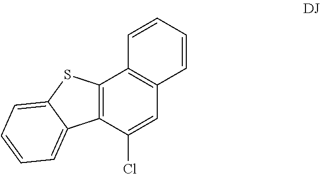 [SMILES: Clc1cc2ccccc2c2sc3ccccc3c12]

Compound DJ was prepared in the same manner as in Preparation Example 21, except that bromobenzene was used instead of 1-bromo-2-chlorobenzene, and (3-chloro-1-(methylthio)naphthalen-2-yl)boronic acid was used instead of (3-(methylthio)naphthalen-2-yl)boronic acid.

### EXAMPLES

### Example 1

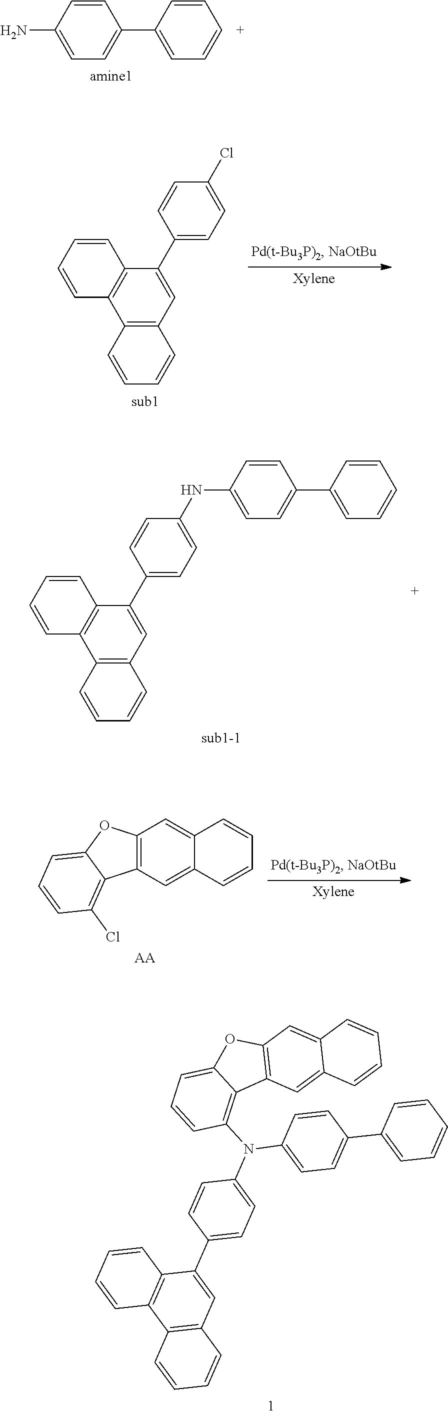

Compound amine1 (10 g, 59.1 mmol), Compound sub1 (17.1 g, 59.1 mmol) and sodium tert-butoxide (7.4 g, 76.8 mmol) were added to xylene (200 ml) under a nitrogen atmosphere, and the mixture was stirred and refluxed. Then, bis(tri-tert-butylphosphine) palladium(0) (0.6 g, 1.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.7 g of Compound sub1-1. (Yield: 63%, MS: [M+H]⁺=422)

Compound sub1-1 (10 g, 23.7 mmol), Compound AA (6 g, 23.7 mmol) and sodium tert-butoxide (15.1 g, 71.2 mmol) were added to xylene (200 ml) under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.8 g of Compound 1. (Yield: 65%, MS: [M+H]⁺=638)

### Example 2

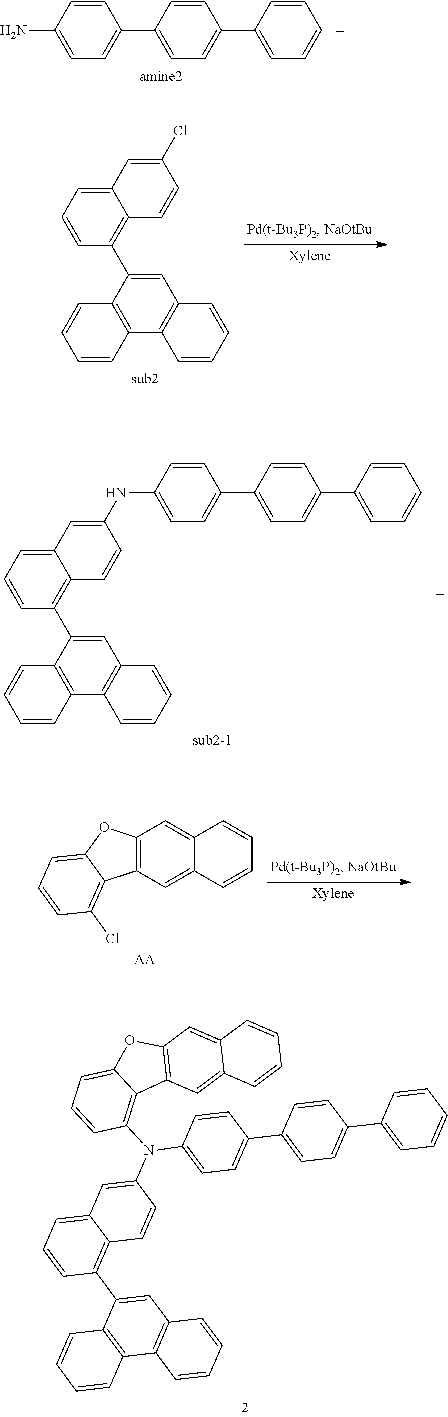

Compound amine2 (10 g, 40.8 mmol), Compound sub2 (13.8 g, 40.8 mmol) and sodium tert-butoxide (5.1 g, 53 mmol) were added to xylene (200 ml) under a nitrogen atmosphere, and the mixture was stirred and refluxed. Then, bis(tri-tert-butylphosphine)palladium(0) (0.4 g, 0.8 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.3 g of Compound sub2-1. (Yield: 64%, MS: [M+H]⁺=548)

Compound sub2-1 (10 g, 18.3 mmol), Compound AA (4.6 g, 18.3 mmol) and sodium tert-butoxide (11.6 g, 54.8 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.8 g of Compound 2. (Yield: 70%, MS: [M+H]⁺=764)

### Example 3

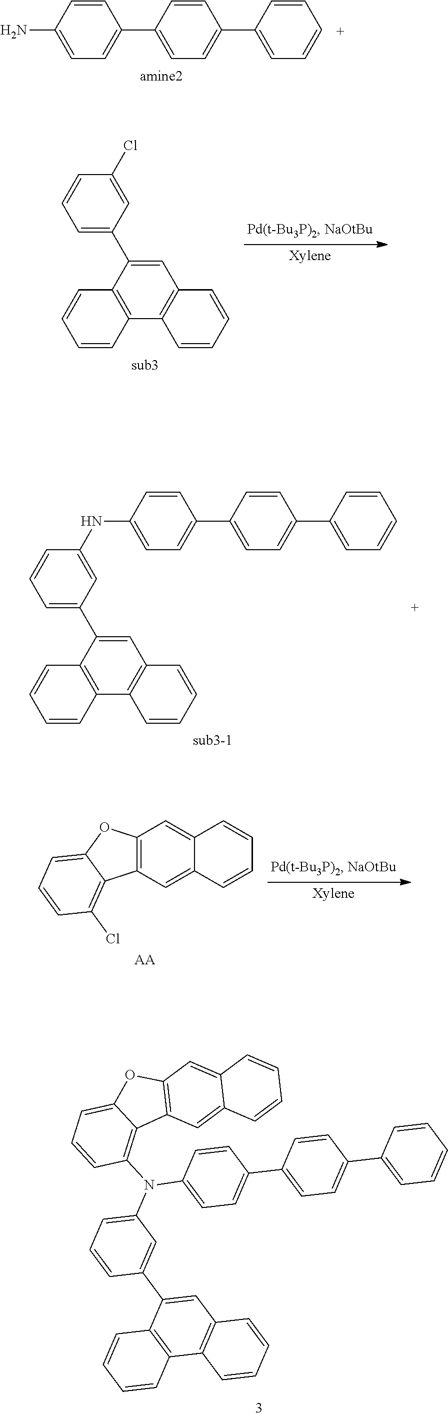

Compound amine2 (10 g, 40.8 mmol), Compound sub3 (11.8 g, 40.8 mmol) and sodium tert-butoxide (5.1 g, 53 mmol) were added to xylene (200 ml) under a nitrogen atmosphere, and the mixture was stirred and refluxed. Then, bis(tri-tert-butylphosphine)palladium(0) (0.4 g, 0.8 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.9 g of Compound sub3-1. (Yield: 54%, MS: [M+H]⁺=498)

Compound sub3-1 (10 g, 20.1 mmol), Compound AA (5.1 g, 20.1 mmol) and sodium tert-butoxide (12.8 g, 60.3 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 37.5 g of Compound 3. (Yield: 52%, MS: [M+H]⁺=714)

### Example 4

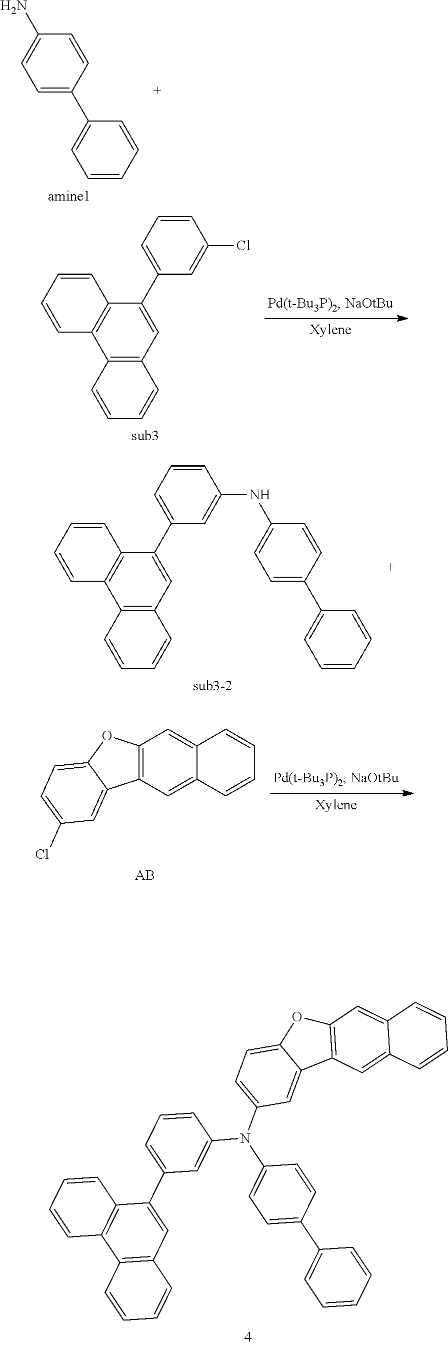 [SMILES: Nc1ccc(-c2ccccc2)cc1 ; Clc1cccc(-c2cc3ccccc3c3ccccc23)c1 ; c1ccc(-c2ccc(Nc3cccc(-c4cc5ccccc5c5ccccc45)c3)cc2)cc1 ; Clc1ccc2oc3cc4ccccc4cc3c2c1 ; "AB" ; c1ccc(-c2ccc(N(c3cccc(-c4cc5ccccc5c5ccccc45)c3)c3ccc4oc5cc6ccccc6cc5c4c3)cc2)cc1]

Compound amine1 (10 g, 59.1 mmol), Compound sub3 (17.1 g, 59.1 mmol) and sodium tert-butoxide (7.4 g, 76.8 mmol) were added to xylene (200 ml) under a nitrogen atmosphere, and the mixture was stirred and refluxed. Then, bis(tri-tert-butylphosphine)palladium(0) (0.6 g, 1.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.9 g of Compound sub3-2. (Yield: 52%, MS: [M+H]⁺=422)

Compound sub3-2 (10 g, 23.7 mmol), Compound AB (6 g, 23.7 mmol) and sodium tert-butoxide (15.1 g, 71.2 mmol) were added to xylene (200 ml) under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.7 g of Compound 4. (Yield: 69%, MS: [M+H]⁺=840)

### Example 5

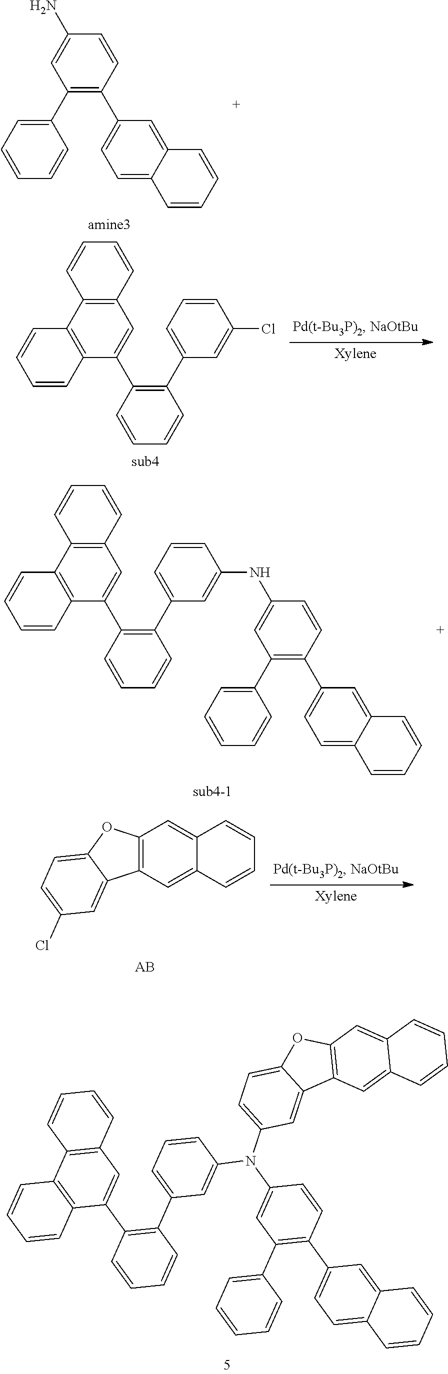 [SMILES: Nc1ccc(-c2ccc3ccccc3c2)c(-c2ccccc2)c1 ; Clc1cccc(-c2ccccc2-c2cc3ccccc3c3ccccc23)c1 ; c1ccc(-c2cc(Nc3cccc(-c4ccccc4-c4cc5ccccc5c5ccccc45)c3)ccc2-c2ccc3ccccc3c2)cc1 ; Clc1ccc2oc3cc4ccccc4cc3c2c1 ; "AB" ; c1ccc(-c2cc(N(c3cccc(-c4ccccc4-c4cc5ccccc5c5ccccc45)c3)c3ccc4oc5cc6ccccc6cc5c4c3)ccc2-c2ccc3ccccc3c2)cc1]

Compound amine3 (10 g, 33.9 mmol), Compound sub4 (12.4 g, 33.9 mmol) and sodium tert-butoxide (4.2 g, 44 mmol) were added to xylene (200 ml) under a nitrogen atmosphere, and the mixture was stirred and refluxed. Then, bis(tri-tert-butylphosphine)palladium(0) (0.3 g, 0.7 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.7 g of Compound sub4-1. (Yield: 60%, MS: [M+H]⁺=624)

Compound sub4-1 (10 g, 16 mmol), Compound AB (4.1 g, 16 mmol) and sodium tert-butoxide (10.2 g, 48.1 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.7 g of Compound 5. (Yield: 57%, MS: [M+H]⁺=840)

### Example 6

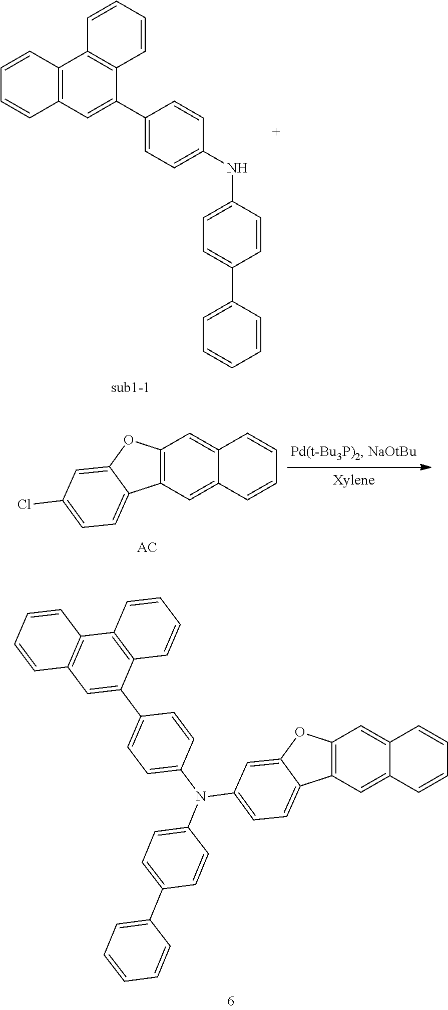 [SMILES: c1ccc(-c2ccc(Nc3ccc(-c4cc5ccccc5c5ccccc45)cc3)cc2)cc1 ; Clc1ccc2c(c1)oc1cc3ccccc3cc12 ; c1ccc(-c2ccc(N(c3ccc(-c4cc5ccccc5c5ccccc45)cc3)c3ccc4c(c3)oc3cc5ccccc5cc34)cc2)cc1]

Compound sub1-1 (10 g, 23.7 mmol), Compound AC (6 g, 23.7 mmol) and sodium tert-butoxide (15.1 g, 71.2 mmol) were added to xylene (200 ml) under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.3 g of Compound 6. (Yield: 68%, MS: [M+H]⁺=638)

### Example 7

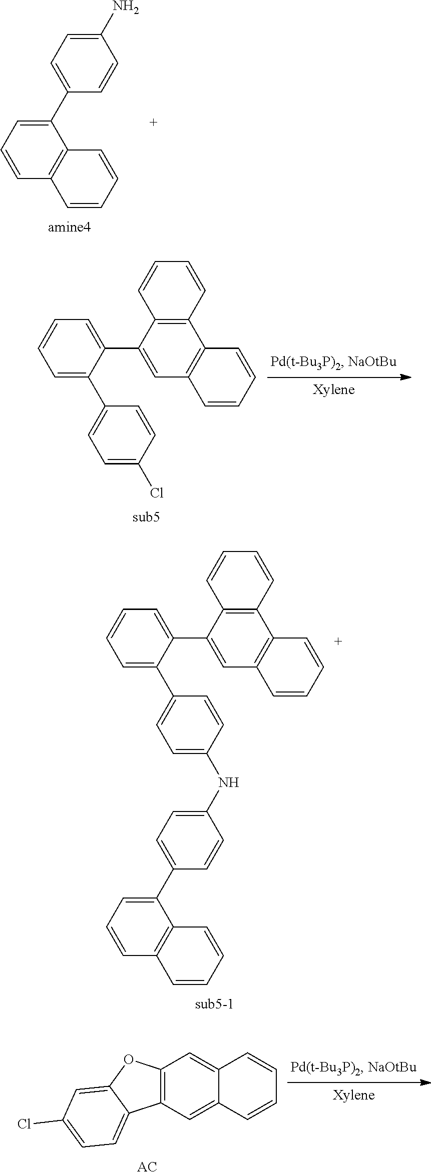 [SMILES: Nc1ccc(-c2cccc3ccccc23)cc1 ; "+" ; Clc1ccc(-c2ccccc2-c2cc3ccccc3c3ccccc23)cc1 ; c1ccc(-c2cc3ccccc3c3ccccc23)c(-c2ccc(Nc3ccc(-c4cccc5ccccc45)cc3)cc2)c1 ; Clc1ccc2c(c1)oc1cc3ccccc3cc12]

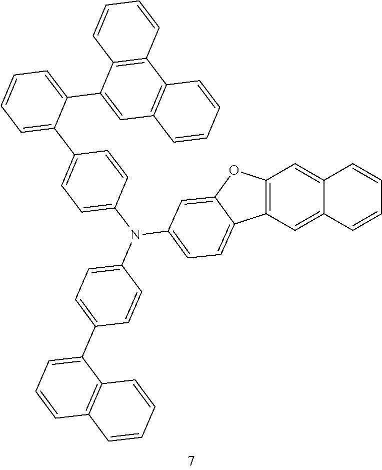 [SMILES: c1ccc(-c2cc3ccccc3c3ccccc23)c(-c2ccc(N(c3ccc(-c4cccc5ccccc45)cc3)c3ccc4c(c3)oc3cc5ccccc5cc34)cc2)c1]

Compound amine4 (10 g, 34.3 mmol), Compound sub5 (12.5 g, 34.3 mmol) and sodium tert-butoxide (4.3 g, 44.6 mmol) were added to xylene (200 ml) under a nitrogen atmosphere, and the mixture was stirred and refluxed. Then, bis(tri-tert-butylphosphine)palladium(0) (0.4 g, 0.7 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10 g of Compound sub5-1. (Yield: 53%, MS: [M+H]⁺=549)

Compound sub5-1 (10 g, 18.3 mmol), Compound AC (4.6 g, 18.3 mmol) and sodium tert-butoxide (11.6 g, 54.8 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.2 g of Compound 7. (Yield: 59%, MS: [M+H]⁺=764)

### Example 8

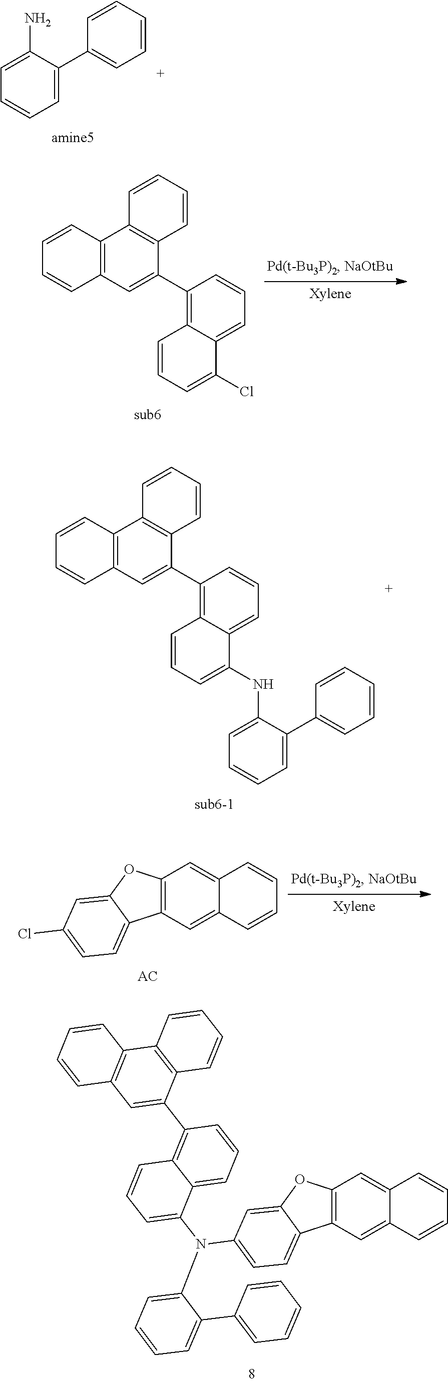 [SMILES: Nc1ccccc1-c1ccccc1 ; Clc1cccc2c(-c3cc4ccccc4c4ccccc34)cccc12 ; c1ccc(-c2ccccc2Nc2cccc3c(-c4cc5ccccc5c5ccccc45)cccc23)cc1 ; Clc1ccc2c(c1)oc1cc3ccccc3cc12 ; c1ccc(-c2ccccc2N(c2ccc3c(c2)oc2cc4ccccc4cc23)c2cccc3c(-c4cc5ccccc5c5ccccc45)cccc23)cc1]

Compound amine5 (10 g, 59.1 mmol), Compound sub6 (20 g, 59.1 mmol) and sodium tert-butoxide (7.4 g, 76.8 mmol) were added to xylene (200 ml) under a nitrogen atmosphere, and the mixture was stirred and refluxed. Then, bis(tri-tert-butylphosphine)palladium(0) (0.6 g, 1.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 17 g of Compound sub6-1. (Yield: 61%, MS: [M+H]⁺=472)

Compound sub6-1 (10 g, 21.2 mmol), Compound AC (5.4 g, 21.2 mmol) and sodium tert-butoxide (13.5 g, 63.6 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.8 g of Compound 8. (Yield: 67%, MS: [M+H]⁺=688)

### Example 9

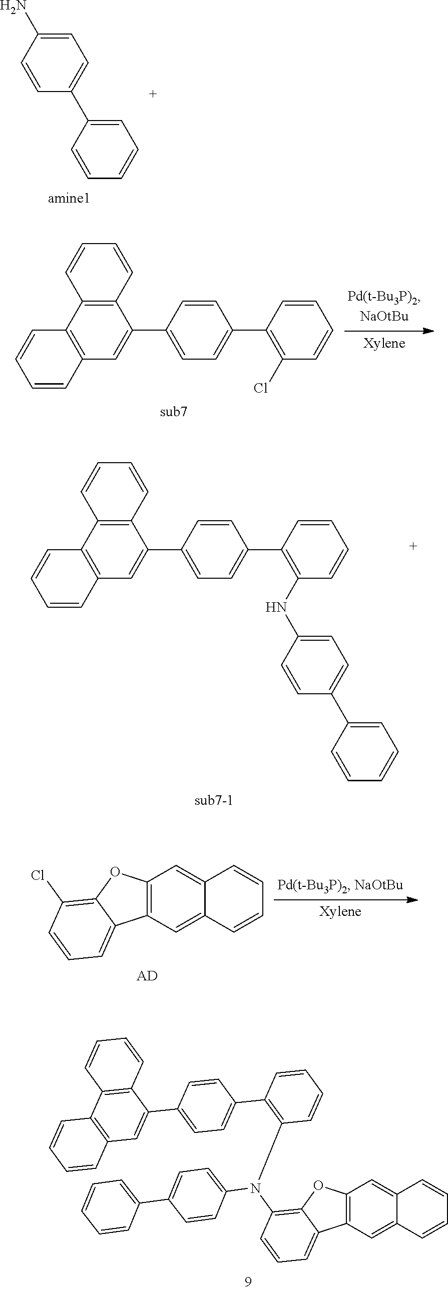

Compound amine1 (10 g, 59.1 mmol), Compound sub1 (21.6 g, 59.1 mmol) and sodium tert-butoxide (7.4 g, 76.8 mmol) were added to xylene (200 ml) under a nitrogen atmosphere, and the mixture was stirred and refluxed. Then, bis(tri-tert-butylphosphine)palladium(0) (0.6 g, 1.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 18.8 g of Compound sub7-1. (Yield: 64%, MS: [M+H]⁺=498)

Compound sub7-1 (10 g, 20.1 mmol), Compound AD (5.1 g, 20.1 mmol) and sodium tert-butoxide (12.8 g, 60.3 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.6 g of Compound 9. (Yield: 60%, MS: [M+H]⁺=714)

### Example 10

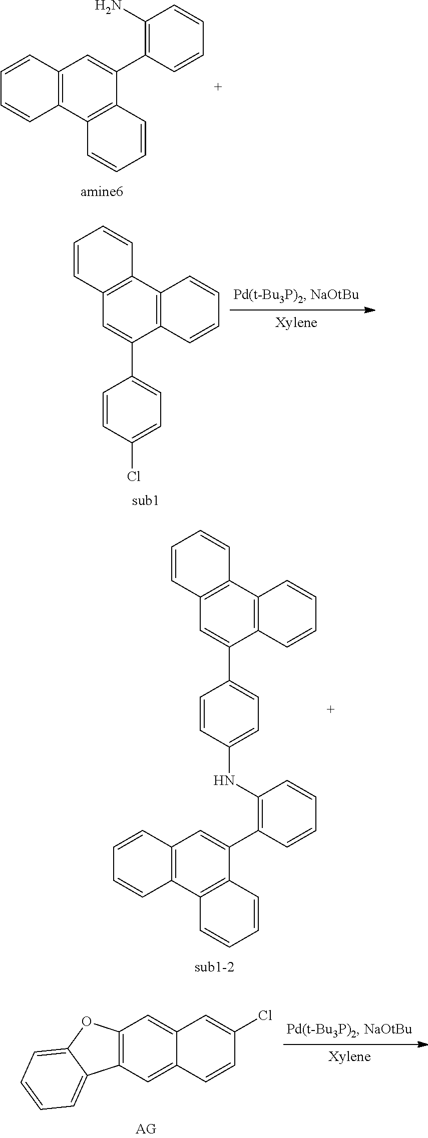 [SMILES: Nc1ccccc1-c1cc2ccccc2c2ccccc12 ; Clc1ccc(-c2cc3ccccc3c3ccccc23)cc1 ; c1ccc(-c2cc3ccccc3c3ccccc23)c(Nc2ccc(-c3cc4ccccc4c4ccccc34)cc2)c1 ; Clc1ccc2cc3c(cc2c1)oc1ccccc13]

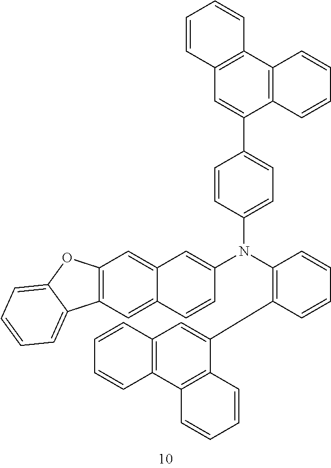 [SMILES: c1ccc(N(c2ccc(-c3cc4ccccc4c4ccccc34)cc2)c2ccc3cc4c(cc3c2)oc2ccccc24)c(-c2cc3ccccc3c3ccccc23)c1]

Compound amine6 (10 g, 37.1 mmol), Compound sub1 (10.7 g, 37.1 mmol) and sodium tert-butoxide (4.6 g, 48.3 mmol) were added to xylene (200 ml) under a nitrogen atmosphere, and the mixture was stirred and refluxed. Then, bis(tri-tert-butylphosphine)palladium(0) (0.4 g, 0.7 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.4 g of Compound sub1-2. (Yield: 69%, MS: [M+H]⁺=522)

Compound sub1-2 (10 g, 19.2 mmol), Compound AG (4.8 g, 19.2 mmol) and sodium tert-butoxide (12.2 g, 57.5 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.2 g of Compound 10. (Yield: 65%, MS: [M+H]⁺=738)

### Example 11

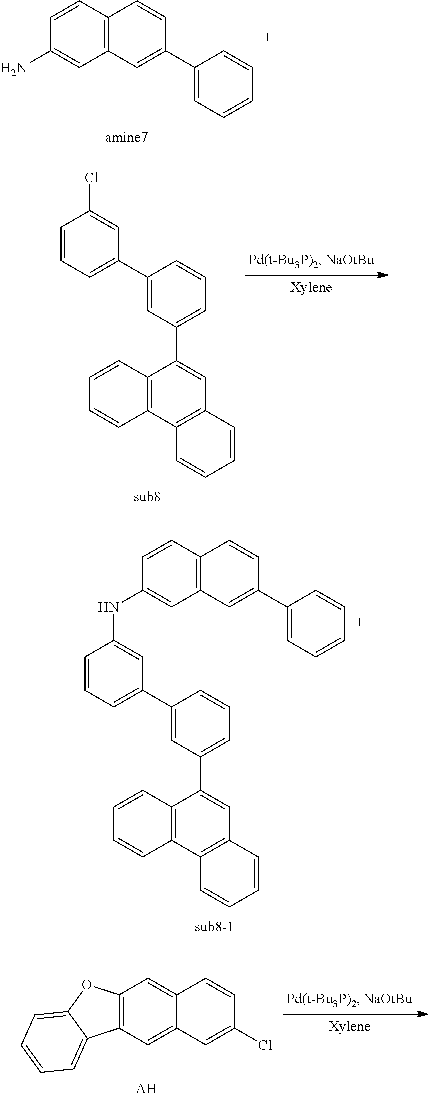

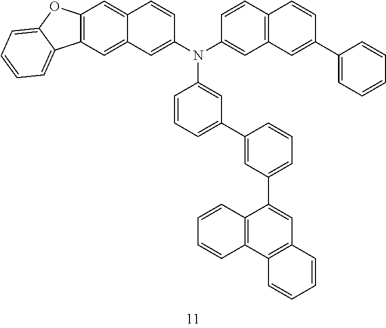 [SMILES: c1ccc(-c2ccc3ccc(N(c4cccc(-c5cccc(-c6cc7ccccc7c7ccccc67)c5)c4)c4ccc5cc6oc7ccccc7c6cc5c4)cc3c2)cc1]

Compound amine7 (10 g, 45.6 mmol), Compound sub8 (16.6 g, 45.6 mmol) and sodium tert-butoxide (5.7 g, 59.3 mmol) were added to xylene (200 ml) under a nitrogen atmosphere, and the mixture was stirred and refluxed. Then, bis(tri-tert-butylphosphine)palladium(0) (0.5 g, 0.9 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.7 g of Compound sub8-1. (Yield: 51%, MS: [M+H]⁺=548)

Compound sub8-1 (10 g, 18.3 mmol), Compound AH (4.6 g, 18.3 mmol) and sodium tert-butoxide (11.6 g, 54.8 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.1 g of Compound 11. (Yield: 58%, MS: [M+H]⁺=764)

### Example 12

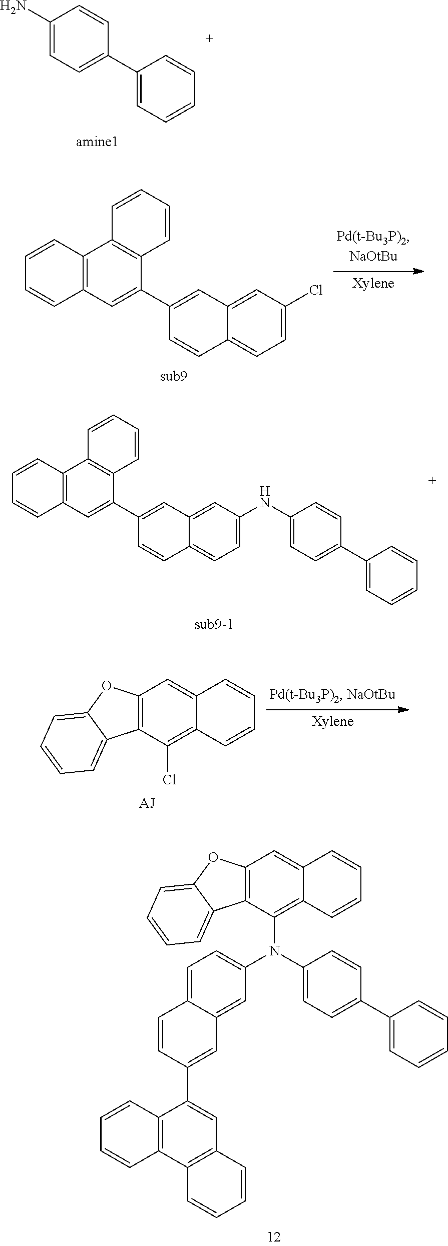 [SMILES: Nc1ccc(-c2ccccc2)cc1 ; Clc1ccc2ccc(-c3cc4ccccc4c4ccccc34)cc2c1 ; c1ccc(-c2ccc(Nc3ccc4ccc(-c5cc6ccccc6c6ccccc56)cc4c3)cc2)cc1 ; Clc1c2ccccc2cc2oc3ccccc3c12 ; c1ccc(-c2ccc(N(c3ccc4ccc(-c5cc6ccccc6c6ccccc56)cc4c3)c3c4ccccc4cc4oc5ccccc5c34)cc2)cc1]

Compound amine1 (10 g, 59.1 mmol), Compound sub9 (20 g, 59.1 mmol) and sodium tert-butoxide (7.4 g, 76.8 mmol) were added to xylene (200 ml) under a nitrogen atmosphere, and the mixture was stirred and refluxed. Then, bis(tri-tert-butylphosphine)palladium(0) (0.6 g, 1.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 19.5 g of Compound sub9-1. (Yield: 70%, MS: [M+H]⁺=472)

Compound sub9-1 (10 g, 21.2 mmol), Compound AJ (5.4 g, 21.2 mmol) and sodium tert-butoxide (13.5 g, 63.6 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.6 g of Compound 12. (Yield: 59%, MS: [M+H]⁺=688)

### Example 13

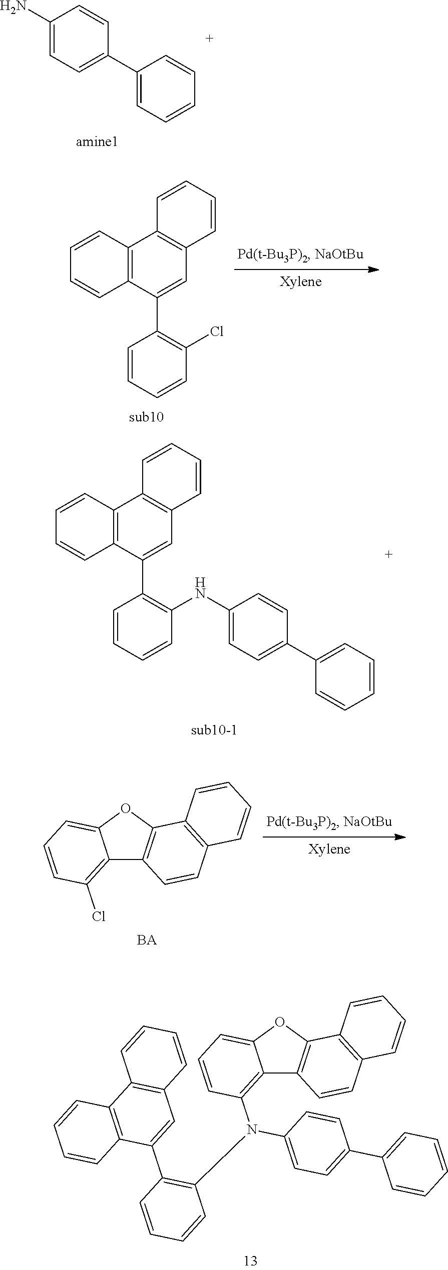 [SMILES: Nc1ccc(-c2ccccc2)cc1 ; Clc1ccccc1-c1cc2ccccc2c2ccccc12 ; c1ccc(-c2ccc(Nc3ccccc3-c3cc4ccccc4c4ccccc34)cc2)cc1 ; Clc1cccc2oc3c4ccccc4ccc3c12 ; c1ccc(-c2ccc(N(c3ccccc3-c3cc4ccccc4c4ccccc34)c3cccc4oc5c6ccccc6ccc5c34)cc2)cc1]

Compound amine1 (10 g, 59.1 mmol), Compound sub10 (17.1 g, 59.1 mmol) and sodium tert-butoxide (7.4 g, 76.8 mmol) were added to xylene (200 ml) under a nitrogen atmosphere, and the mixture was stirred and refluxed. Then, bis(tri-tert-butylphosphine)palladium(0) (0.6 g, 1.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.2 g of Compound sub10-1. (Yield: 53%, MS: [M+H]⁺=422)

Compound sub10-1 (10 g, 23.7 mmol), Compound BA (6 g, 23.7 mmol) and sodium tert-butoxide (15.1 g, 71.2 mmol) were added to xylene (200 ml) under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.5 g of Compound 13. (Yield: 63%, MS: [M+H]⁺=638)

### Example 14

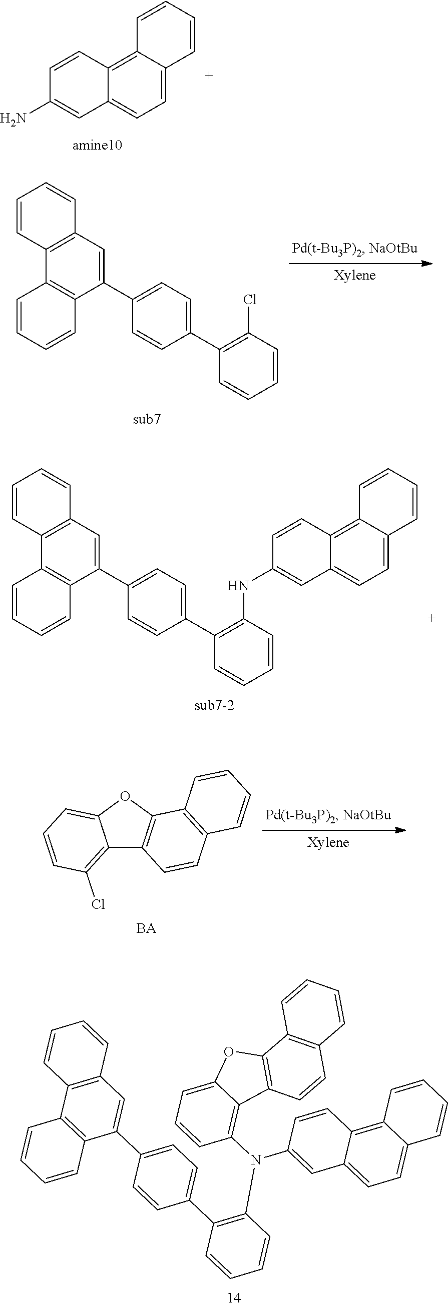 [SMILES: Nc1ccc2c(ccc3ccccc32)c1 ; Clc1ccccc1-c1ccc(-c2cc3ccccc3c3ccccc23)cc1 ; c1ccc(-c2ccc(-c3cc4ccccc4c4ccccc34)cc2)c(Nc2ccc3c(ccc4ccccc43)c2)c1 ; Clc1cccc2oc3c4ccccc4ccc3c12 ; c1ccc(N(c2ccc3c(ccc4ccccc43)c2)c2cccc3oc4c5ccccc5ccc4c23)c(-c2ccc(-c3cc4ccccc4c4ccccc34)cc2)c1]

Compound amine10 (10 g, 51.7 mmol), Compound sub7 (18.9 g, 51.7 mmol) and sodium tert-butoxide (6.5 g, 67.3 mmol) were added to xylene (200 ml) under a nitrogen atmosphere, and the mixture was stirred and refluxed. Then, bis(tri-tert-butylphosphine)palladium(0) (0.5 g, 1 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.5 g of Compound sub7-2. (Yield: 50%, MS: [M+H]⁺=522)

Compound sub7-2 (10 g, 19.2 mmol), Compound BA (4.8 g, 19.2 mmol) and sodium tert-butoxide (12.2 g, 57.5 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.8 g of Compound 14. (Yield: 62%, MS: [M+H]⁺=738)

### Example 15

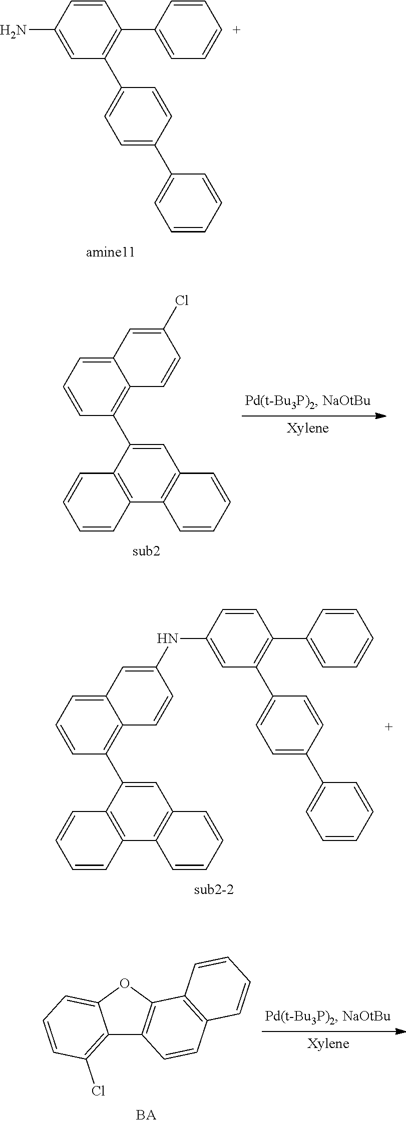 [SMILES: Nc1ccc(C2=CC=[C+]C=C2)c(-c2ccc(-c3ccccc3)cc2)c1 ; Clc1ccc2c(-c3cc4ccccc4c4ccccc34)cccc2c1 ; c1ccc(-c2ccc(-c3cc(Nc4ccc5c(-c6cc7ccccc7c7ccccc67)cccc5c4)ccc3-c3ccccc3)cc2)cc1 ; Clc1cccc2oc3c4ccccc4ccc3c12]

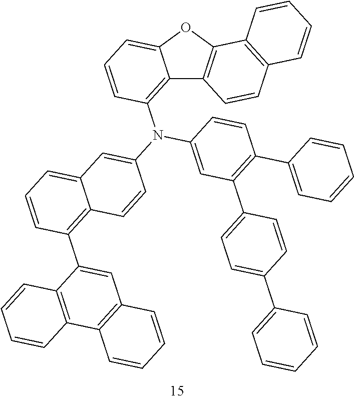 [SMILES: c1ccc(-c2ccc(-c3cc(N(c4ccc5c(-c6cc7ccccc7c7ccccc67)cccc5c4)c4cccc5oc6c7ccccc7ccc6c45)ccc3-c3ccccc3)cc2)cc1]

Compound amine11 (10 g, 31.1 mmol), Compound sub2 (10.5 g, 31.1 mmol) and sodium tert-butoxide (3.9 g, 40.4 mmol) were added to xylene (200 ml) under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.8 g of Compound sub2-2. (Yield: 61%, MS: [M+H]⁺=624)

Compound sub2-2 (10 g, 16 mmol), Compound BA (4.1 g, 16 mmol) and sodium tert-butoxide (10.2 g, 48.1 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.4 g of Compound 15. (Yield: 70%, MS: [M+H]⁺=840)

### Example 16

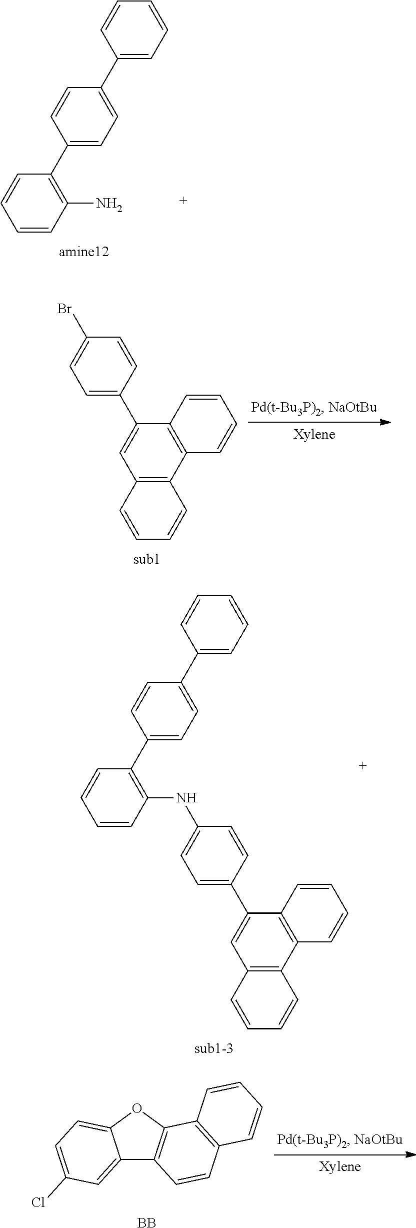 [SMILES: Nc1ccccc1-c1ccc(-c2ccccc2)cc1 ; Brc1ccc(-c2cc3ccccc3c3ccccc23)cc1 ; c1ccc(-c2ccc(-c3ccccc3Nc3ccc(-c4cc5ccccc5c5ccccc45)cc3)cc2)cc1 ; Clc1ccc2oc3c4ccccc4ccc3c2c1 ; "BB"]

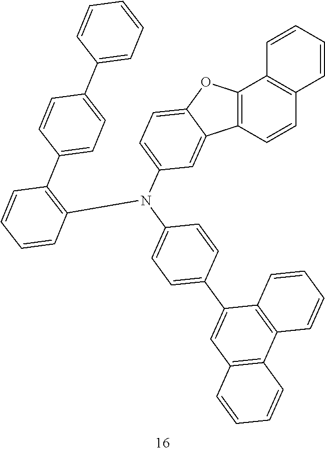 [SMILES: c1ccc(-c2ccc(-c3ccccc3N(c3ccc(-c4cc5ccccc5c5ccccc45)cc3)c3ccc4oc5c6ccccc6ccc5c4c3)cc2)cc1]

Compound amine12 (10 g, 40.8 mmol), Compound sub1 (11.8 g, 40.8 mmol) and sodium tert-butoxide (5.1 g, 53 mmol) were added to xylene (200 ml) under a nitrogen atmosphere, and the mixture was stirred and refluxed. Then, bis(tri-tert-butylphosphine)palladium(0) (0.4 g, 0.8 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.3 g of Compound sub1-3. (Yield: 51%, MS: [M+H]⁺=498)

Compound sub1-3 (10 g, 20.1 mmol), Compound BB (5.1 g, 20.1 mmol) and sodium tert-butoxide (12.8 g, 60.3 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.7 g of Compound 16. (Yield: 68%, MS: [M+H]⁺=714)

### Example 17

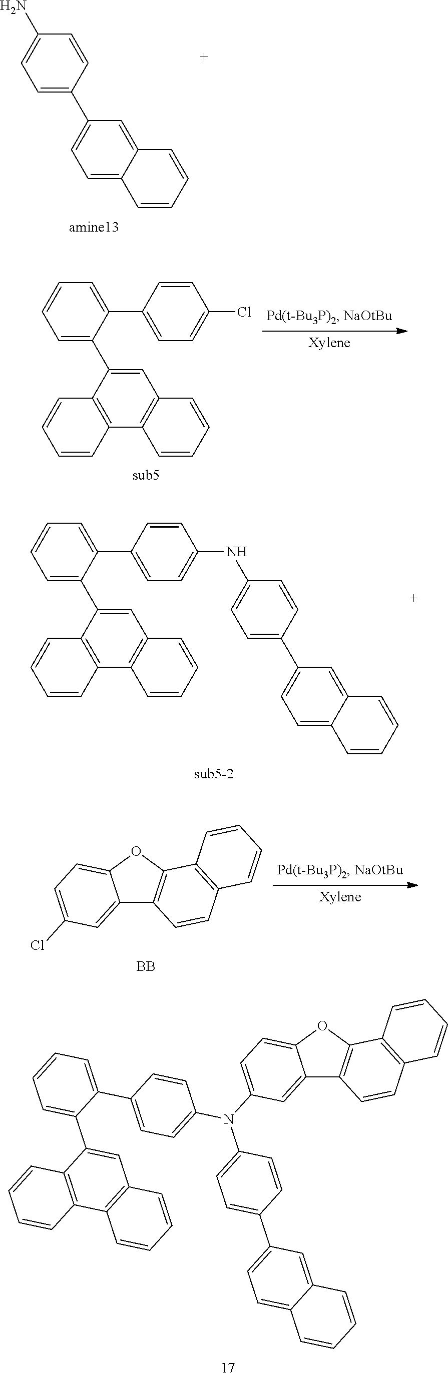 [SMILES: Nc1ccc(-c2ccc3ccccc3c2)cc1 ; Clc1ccc(-c2ccccc2-c2cc3ccccc3c3ccccc23)cc1 ; c1ccc(-c2cc3ccccc3c3ccccc23)c(-c2ccc(Nc3ccc(-c4ccc5ccccc5c4)cc3)cc2)c1 ; Clc1ccc2oc3c4ccccc4ccc3c2c1 ; "BB" ; c1ccc(-c2cc3ccccc3c3ccccc23)c(-c2ccc(N(c3ccc(-c4ccc5ccccc5c4)cc3)c3ccc4oc5c6ccccc6ccc5c4c3)cc2)c1]

Compound amine13 (10 g, 45.6 mmol), Compound sub5 (16.6 g, 45.6 mmol) and sodium tert-butoxide (5.7 g, 59.3 mmol) were added to xylene (200 ml) under a nitrogen atmosphere, and the mixture was stirred and refluxed. Then, bis(tri-tert-butylphosphine)palladium(0) (0.5 g, 0.9 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.7 g of Compound sub5-2. (Yield: 59%, MS: [M+H]⁺=548)

Compound sub5-2 (10 g, 18.3 mmol), Compound BB (4.6 g, 18.3 mmol) and sodium tert-butoxide (11.6 g, 54.8 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.8 g of Compound 17. (Yield: 56%, MS: [M+H]⁺=764)

### Example 18

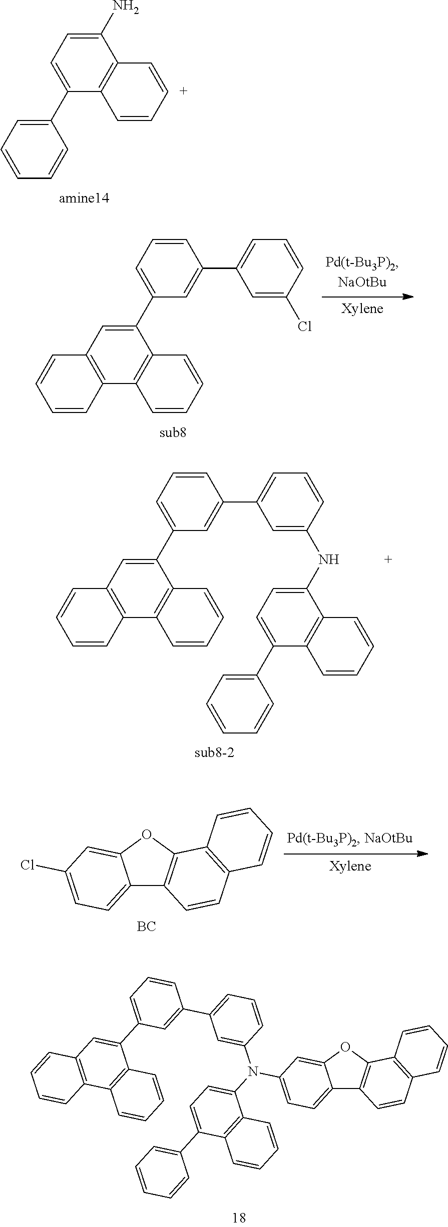

Compound amine14 (10 g, 45.6 mmol), Compound sub8 (16.6 g, 45.6 mmol) and sodium tert-butoxide (5.7 g, 59.3 mmol) were added to xylene (200 ml) under a nitrogen atmosphere, and the mixture was stirred and refluxed. Then, bis(tri-tert-butylphosphine)palladium(0) (0.5 g, 0.9 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16 g of Compound sub8-2. (Yield: 59%, MS: [M+H]⁺=548)

Compound sub8-2 (10 g, 18.3 mmol), Compound BC (4.6 g, 18.3 mmol) and sodium tert-butoxide (11.6 g, 54.8 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.1 g of Compound 18. (Yield: 58%, MS: [M+H]⁺=764)

### Example 19

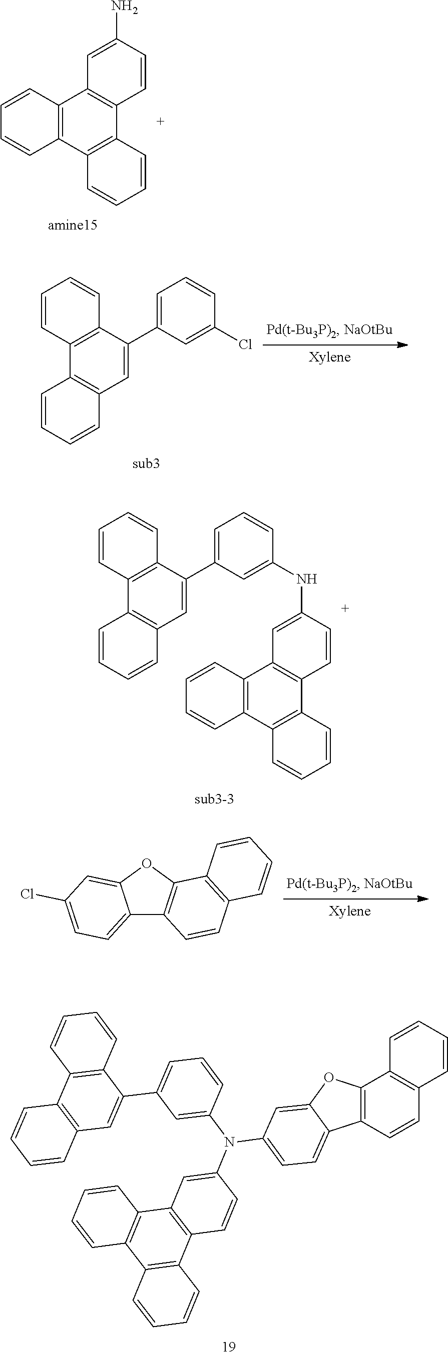 [SMILES: Nc1ccc2c3ccccc3c3ccccc3c2c1 ; Clc1cccc(-c2cc3ccccc3c3ccccc23)c1 ; c1cc(Nc2ccc3c4ccccc4c4ccccc4c3c2)cc(-c2cc3ccccc3c3ccccc23)c1 ; Clc1ccc2c(c1)oc1c3ccccc3ccc21 ; c1cc(-c2cc3ccccc3c3ccccc23)cc(N(c2ccc3c(c2)oc2c4ccccc4ccc32)c2ccc3c4ccccc4c4ccccc4c3c2)c1]

Compound amine15 (10 g, 41.1 mmol), Compound sub3 (11.9 g, 41.1 mmol) and sodium tert-butoxide (5.1 g, 53.4 mmol) were added to xylene (200 ml) under a nitrogen atmosphere, and the mixture was stirred and refluxed. Then, bis(tri-tert-butylphosphine)palladium(0) (0.4 g, 0.8 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.2 g of Compound sub3-3. (Yield: 70%, MS: [M+H]⁺=496)

Compound sub3-3 (10 g, 20.2 mmol), Compound BC (5.1 g, 20.2 mmol) and sodium tert-butoxide (12.8 g, 60.5 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.7 g of Compound 19. (Yield: 54%, MS: [M+H]⁺=712)

### Example 20

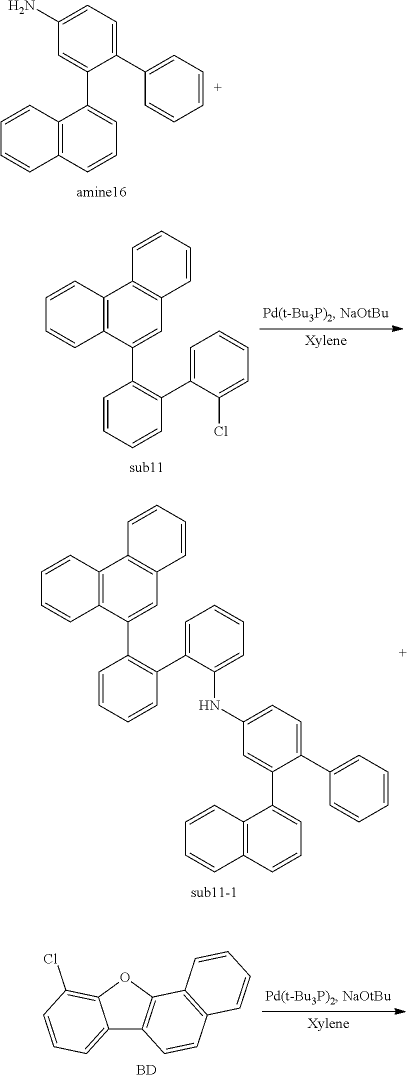

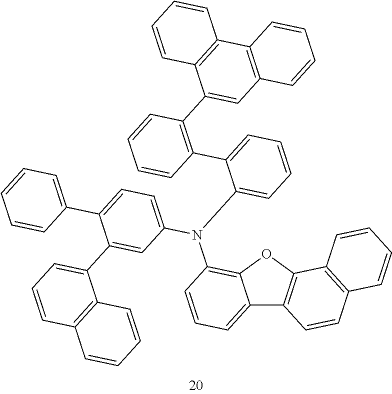 [SMILES: c1ccc(-c2ccc(N(c3ccccc3-c3ccccc3-c3cc4ccccc4c4ccccc34)c3cccc4c3oc3c5ccccc5ccc43)cc2-c2cccc3ccccc23)cc1]

Compound amine16 (10 g, 33.9 mmol), Compound sub11 (12.4 g, 33.9 mmol) and sodium tert-butoxide (4.2 g, 44 mmol) were added to xylene (200 ml) under a nitrogen atmosphere, and the mixture was stirred and refluxed. Then, bis(tri-tert-butylphosphine)palladium(0) (0.3 g, 0.7 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12 g of Compound sub11-1. (Yield: 57%, MS: [M+H]⁺=624)

Compound sub11-1 (10 g, 16 mmol), Compound BD (4.1 g, 16 mmol) and sodium tert-butoxide (10.2 g, 48.1 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.6 g of Compound 20. (Yield: 64%, MS: [M+H]⁺=840)

### Example 21

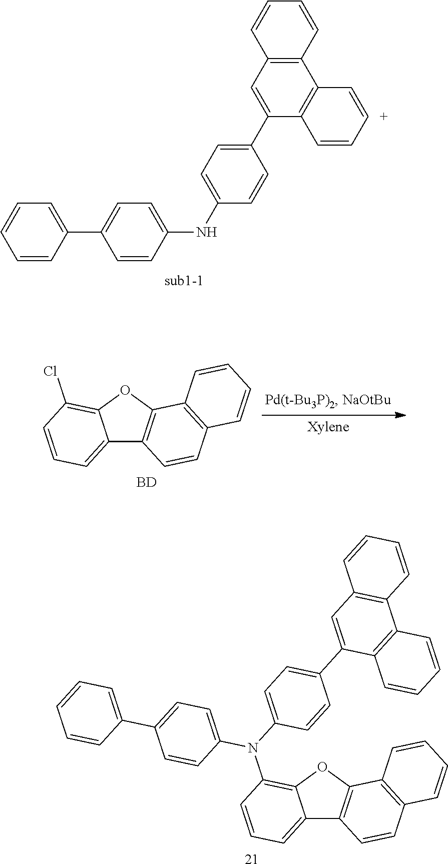

Compound sub1-1 (10 g, 23.7 mmol), Compound BD (6 g, 23.7 mmol) and sodium tert-butoxide (15.1 g, 71.2 mmol) were added to xylene (200 ml) under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.5 g of Compound 21. (Yield: 56%, MS: [M+H]⁺=638)

### Example 22

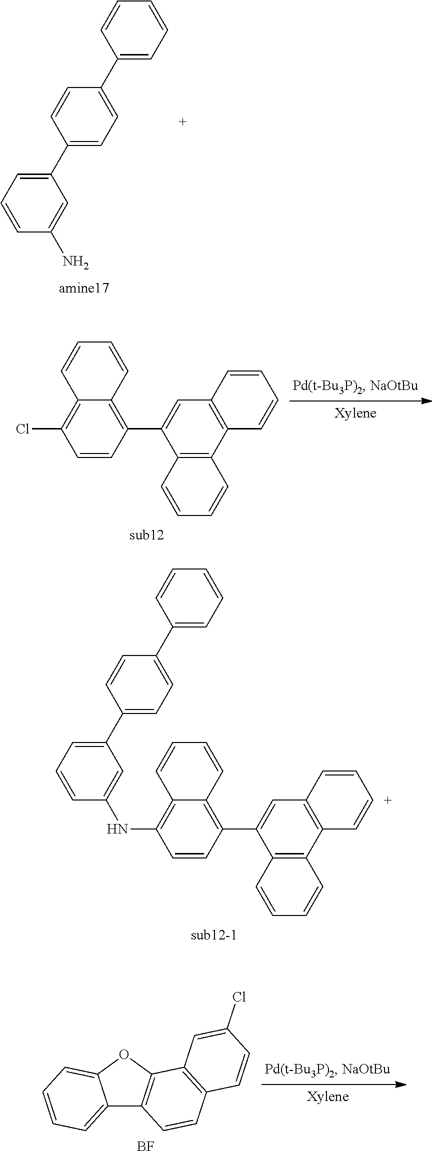

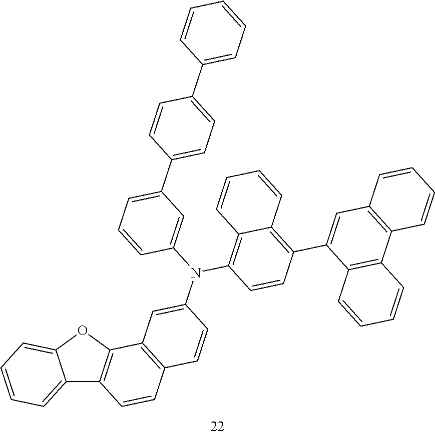 [SMILES: c1ccc(-c2ccc(-c3cccc(N(c4ccc5ccc6c7ccccc7oc6c5c4)c4ccc(-c5cc6ccccc6c6ccccc56)c5ccccc45)c3)cc2)cc1]

Compound amine17 (10 g, 40.8 mmol), Compound sub12 (13.8 g, 40.8 mmol) and sodium tert-butoxide (5.1 g, 53 mmol) were added to xylene (200 ml) under a nitrogen atmosphere, and the mixture was stirred and refluxed. Then, bis(tri-tert-butylphosphine)palladium(0) (0.4 g, 0.8 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.9 g of Compound sub12-1. (Yield: 58%, MS: [M+H]⁺=548)

Compound sub12-1 (10 g, 18.3 mmol), Compound BF (4.6 g, 18.3 mmol) and sodium tert-butoxide (11.6 g, 54.8 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7 g of Compound 22. (Yield: 50%, MS: [M+H]⁺=764)

### Example 23

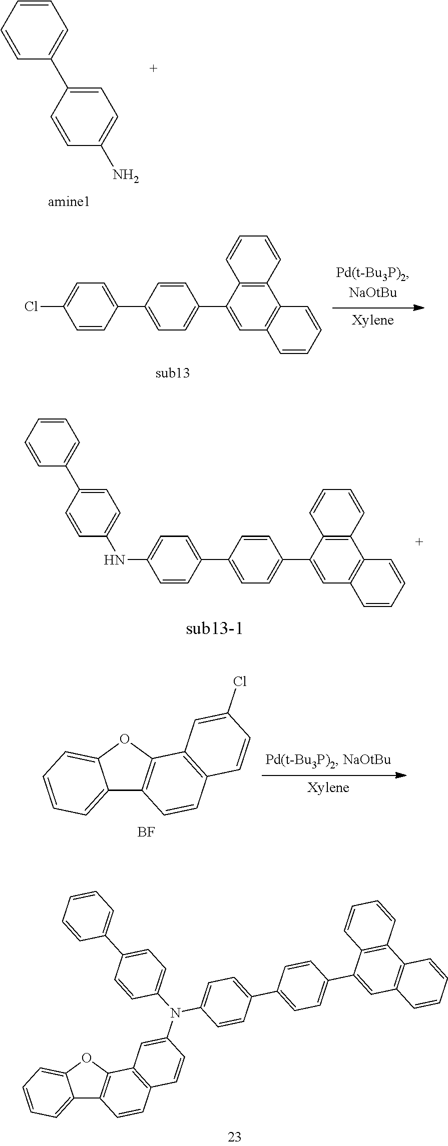

Compound amine1 (10 g, 59.1 mmol), Compound sub13 (21.6 g, 59.1 mmol) and sodium tert-butoxide (7.4 g, 76.8 mmol) were added to xylene (200 ml) under a nitrogen atmosphere, and the mixture was stirred and refluxed. Then, bis(tri-tert-butylphosphine)palladium(0) (0.6 g, 1.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15 g of Compound sub13-1. (Yield: 51%, MS: [M+H]⁺=498)

Compound sub13-1 (10 g, 20.1 mmol), Compound BF (5.1 g, 20.1 mmol) and sodium tert-butoxide (12.8 g, 60.3 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.6 g of Compound 23. (Yield: 53%, MS: [M+H]⁺=714)

### Example 24

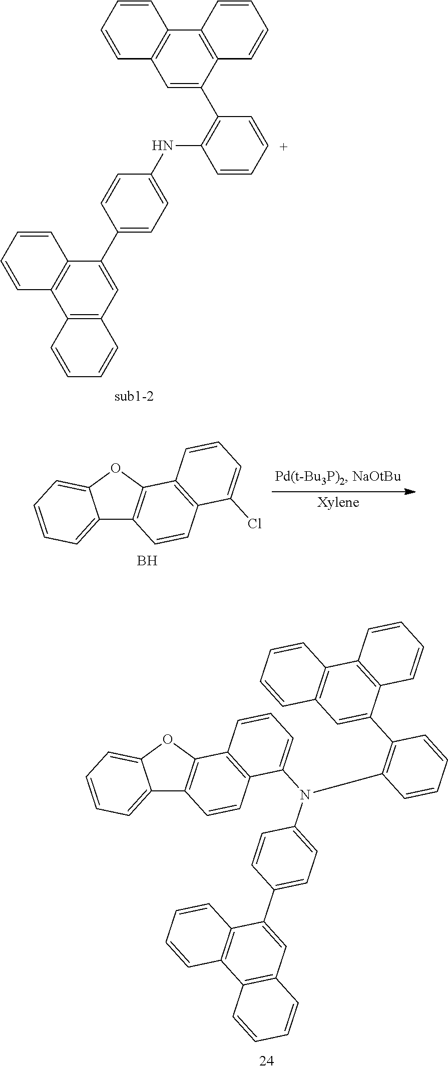

Compound sub1-2 (10 g, 19.2 mmol), Compound BH (4.8 g, 19.2 mmol) and sodium tert-butoxide (12.2 g, 57.5 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.9 g of Compound 24. (Yield: 63%, MS: [M+H]⁺=738)

### Example 25

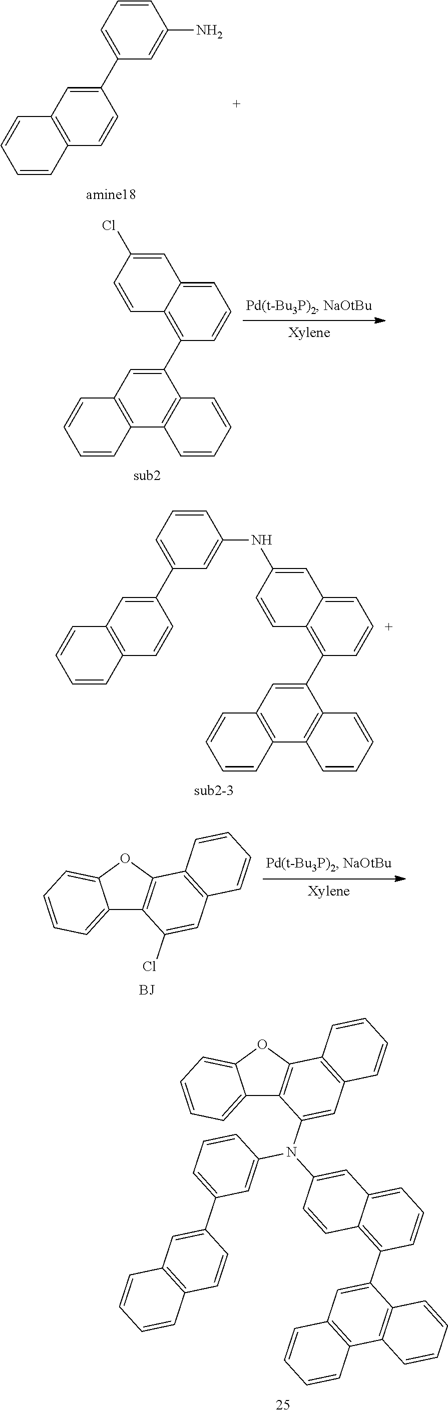 [SMILES: Nc1cccc(-c2ccc3ccccc3c2)c1 ; "+" ; Clc1ccc2c(-c3cc4ccccc4c4ccccc34)cccc2c1 ; [C+]1=CC(c2cc3ccccc3c3ccccc23)=c2ccc(Nc3cccc(-c4ccc5ccccc5c4)c3)cc2=C1 ; Clc1cc2ccccc2c2oc3ccccc3c12 ; c1cc(-c2ccc3ccccc3c2)cc(N(c2ccc3c(-c4cc5ccccc5c5ccccc45)cccc3c2)c2cc3ccccc3c3oc4ccccc4c23)c1]

Compound amine18 (10 g, 45.6 mmol), Compound sub2 (15.5 g, 45.6 mmol) and sodium tert-butoxide (5.7 g, 59.3 mmol) were added to xylene (200 ml) under a nitrogen atmosphere, and the mixture was stirred and refluxed. Then, bis(tri-tert-butylphosphine)palladium(0) (0.5 g, 0.9 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.9 g of Compound sub2-3. (Yield: 50%, MS: [M+H]⁺=522)

Compound sub2-3 (10 g, 19.2 mmol), Compound BJ (4.8 g, 19.2 mmol) and sodium tert-butoxide (12.2 g, 57.5 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.5 g of Compound 25. (Yield: 53%, MS: [M+H]⁺=738)

### Example 26

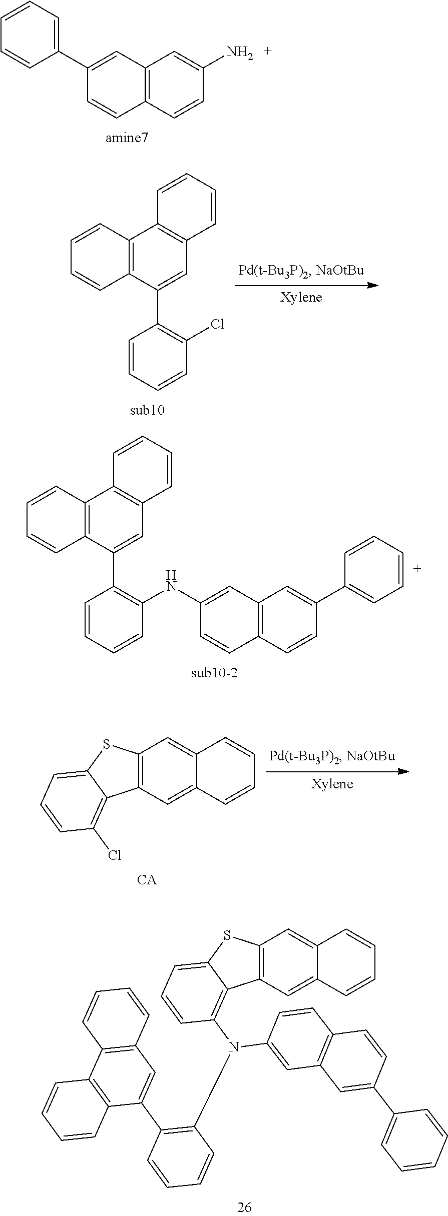

Compound amine7 (10 g, 45.6 mmol), Compound sub10 (13.2 g, 45.6 mmol) and sodium tert-butoxide (5.7 g, 59.3 mmol) were added to xylene (200 ml) under a nitrogen atmosphere, and the mixture was stirred and refluxed. Then, bis(tri-tert-butylphosphine)palladium(0) (0.5 g, 0.9 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.2 g of Compound sub10-2. (Yield: 66%, MS: [M+H]⁺=472)

Compound sub10-2 (10 g, 21.2 mmol), Compound CA (5.7 g, 21.2 mmol) and sodium tert-butoxide (13.5 g, 63.6 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.2 g of Compound 26. (Yield: 62%, MS: [M+H]⁺=704)

### Example 27

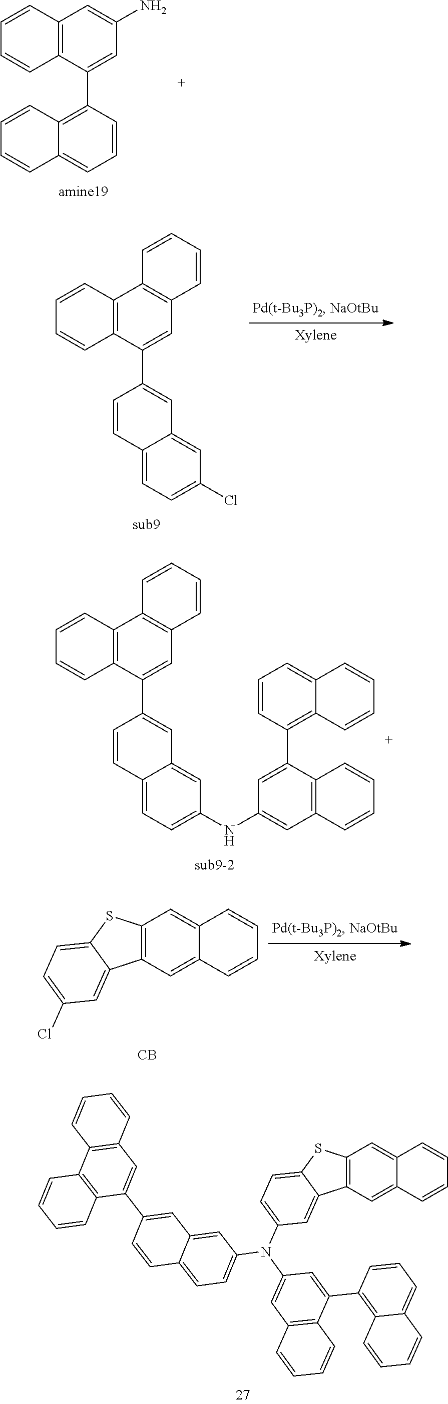

Compound amine19 (10 g, 37.1 mmol), Compound sub9 (12.6 g, 37.1 mmol) and sodium tert-butoxide (4.6 g, 48.3 mmol) were added to xylene (200 ml) under a nitrogen atmosphere, and the mixture was stirred and refluxed. Then, bis(tri-tert-butylphosphine)palladium(0) (0.4 g, 0.7 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11 g of Compound sub9-2. (Yield: 52%, MS: [M+H]⁺=572)

Compound sub9-2 (10 g, 17.5 mmol), Compound CB (4.4 g, 17.5 mmol) and sodium tert-butoxide (11.1 g, 52.5 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7 g of Compound 27. (Yield: 50%, MS: [M+H]⁺=804)

### Example 28

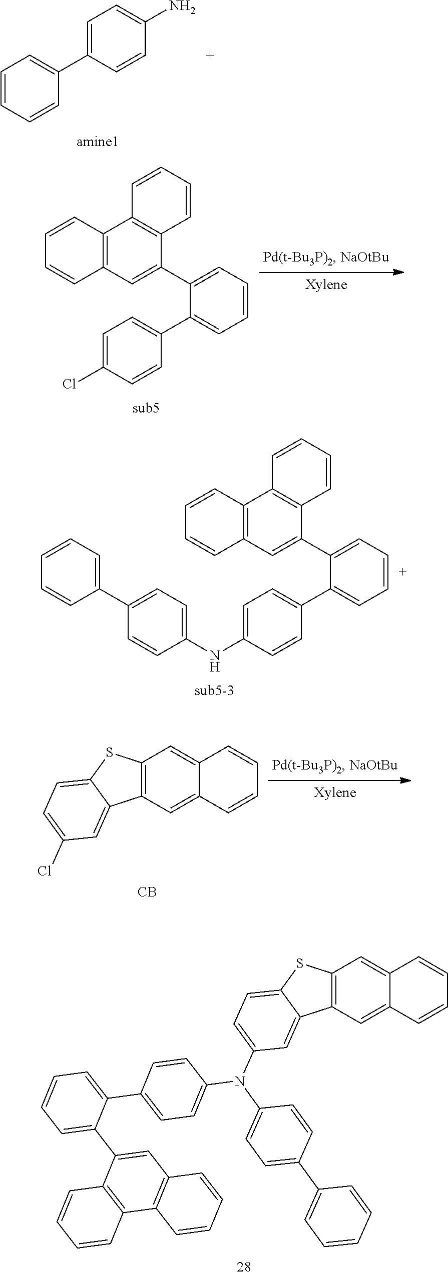

Compound amine1 (10 g, 59.1 mmol), Compound sub5 (21.6 g, 59.1 mmol) and sodium tert-butoxide (7.4 g, 76.8 mmol) were added to xylene (200 ml) under a nitrogen atmosphere, and the mixture was stirred and refluxed. Then, bis(tri-tert-butylphosphine)palladium(0) (0.6 g, 1.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 20.3 g of Compound sub5-3. (Yield: 69%, MS: [M+H]⁺=498)

Compound sub5-3 (10 g, 20.1 mmol), Compound CB (5.4 g, 20.1 mmol) and sodium tert-butoxide (12.8 g, 60.3 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.8 g of Compound 28. (Yield: 53%, MS: [M+H]⁺=730)

### Example 29

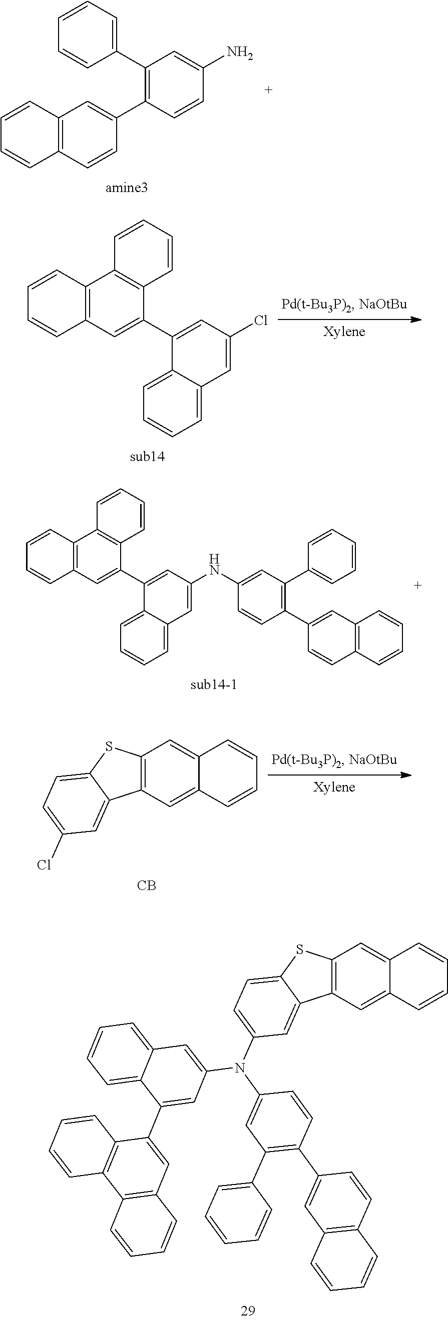

Compound amine3 (10 g, 33.9 mmol), Compound sub14 (11.5 g, 33.9 mmol) and sodium tert-butoxide (4.2 g, 44 mmol) were added to xylene (200 ml) under a nitrogen atmosphere, and the mixture was stirred and refluxed. Then, bis(tri-tert-butylphosphine)palladium(0) (0.3 g, 0.7 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.1 g of Compound sub14-1. (Yield: 55%, MS: [M+H]⁺=598)

Compound sub14-1 (10 g, 16.7 mmol), Compound CB (4.5 g, 16.7 mmol) and sodium tert-butoxide (10.7 g, 50.2 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.1 g of Compound 29. (Yield: 51%, MS: [M+H]⁺=830)

### Example 30

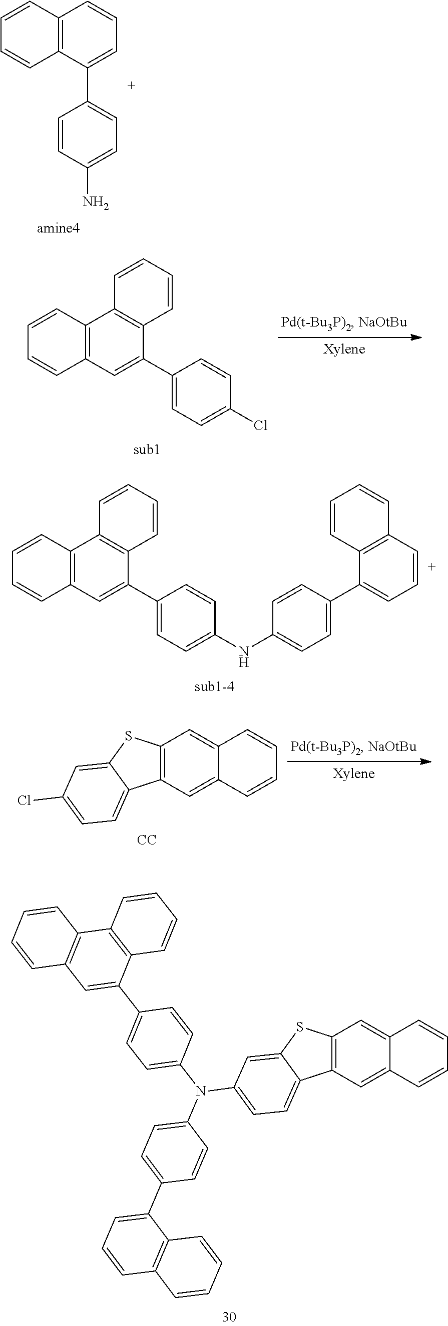

Compound amine4 (10 g, 45.6 mmol), Compound sub1 (13.2 g, 45.6 mmol) and sodium tert-butoxide (5.7 g, 59.3 mmol) were added to xylene (200 ml) under a nitrogen atmosphere, and the mixture was stirred and refluxed. Then, bis(tri-tert-butylphosphine)palladium(0) (0.5 g, 0.9 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.1 g of Compound sub1-4. (Yield: 61%, MS: [M+H]⁺=472)

Compound sub1-4 (10 g, 21.2 mmol), Compound CC (5.7 g, 21.2 mmol) and sodium tert-butoxide (13.5 g, 63.6 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.8 g of Compound 30. (Yield: 66%, MS: [M+H]⁺=704)

### Example 31

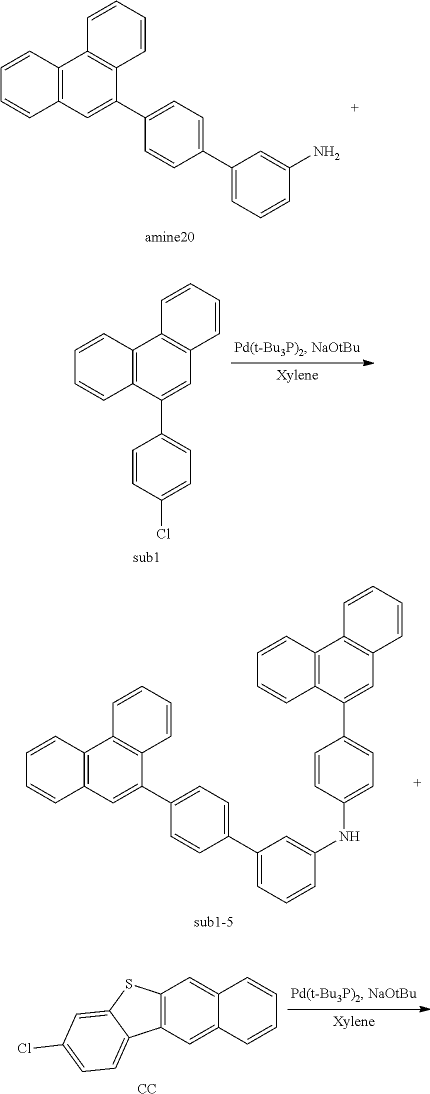

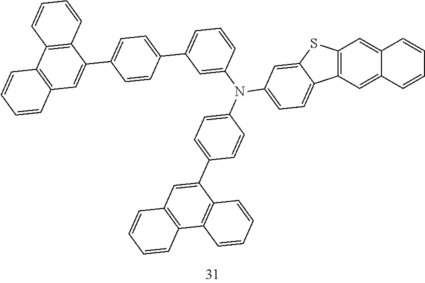 [SMILES: c1cc(-c2ccc(-c3cc4ccccc4c4ccccc34)cc2)cc(N(c2ccc(-c3cc4ccccc4c4ccccc34)cc2)c2ccc3c(c2)sc2cc4ccccc4cc23)c1]

Compound amine 20 (10 g, 28.9 mmol), Compound sub1 (8.4 g, 28.9 mmol) and sodium tert-butoxide (3.6 g, 37.6 mmol) were added to xylene (200 ml) under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.1 g of Compound sub1-5. (Yield: 64%, MS: [M+H]⁺=598)

Compound sub1-5 (10 g, 16.7 mmol), Compound CC (4.5 g, 16.7 mmol) and sodium tert-butoxide (10.7 g, 50.2 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.8 g of Compound 31. (Yield: 56%, MS: [M+H]⁺=830)

### Example 32

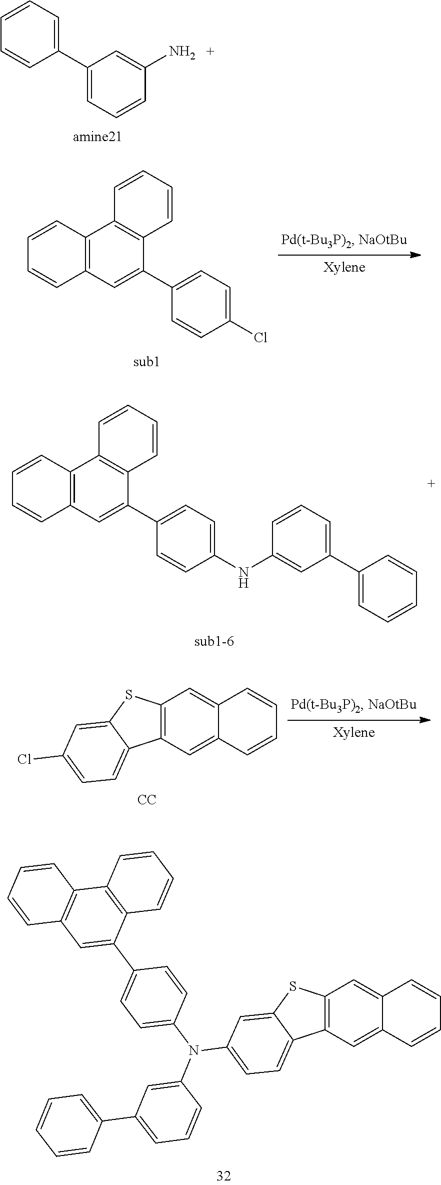

Compound amine21 (10 g, 59.1 mmol), Compound sub1 (17.1 g, 59.1 mmol) and sodium tert-butoxide (7.4 g, 76.8 mmol) were added to xylene (200 ml) under a nitrogen atmosphere, and the mixture was stirred and refluxed. Then, bis(tri-tert-butylphosphine)palladium(0) (0.6 g, 1.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 17.2 g of Compound sub1-6. (Yield: 69%, MS: [M+H]⁺=422)

Compound sub1-6 (10 g, 23.7 mmol), Compound CC (6.4 g, 23.7 mmol) and sodium tert-butoxide (15.1 g, 71.2 mmol) were added to xylene (200 ml) under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.7 g of Compound 32. (Yield: 69%, MS: [M+H]⁺=654)

### Example 33

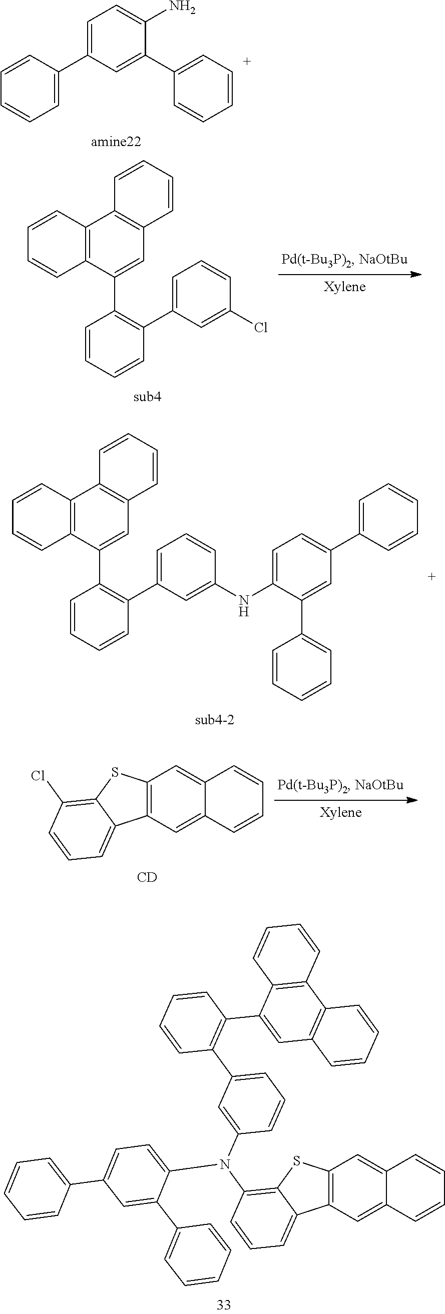

Compound amine22 (10 g, 40.8 mmol), Compound sub4 (14.9 g, 40.8 mmol) and sodium tert-butoxide (5.1 g, 53 mmol) were added to xylene (200 ml) under a nitrogen atmosphere, and the mixture was stirred and refluxed. Then, bis(tri-tert-butylphosphine)palladium(0) (0.4 g, 0.8 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.1 g of Compound sub4-2. (Yield: 56%, MS: [M+H]⁺=574)

Compound sub4-2 (10 g, 17.4 mmol), Compound CD (4.7 g, 17.4 mmol) and sodium tert-butoxide (11.1 g, 52.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.3 g of Compound 33. (Yield: 52%, MS: [M+H]⁺=80)

### Example 34

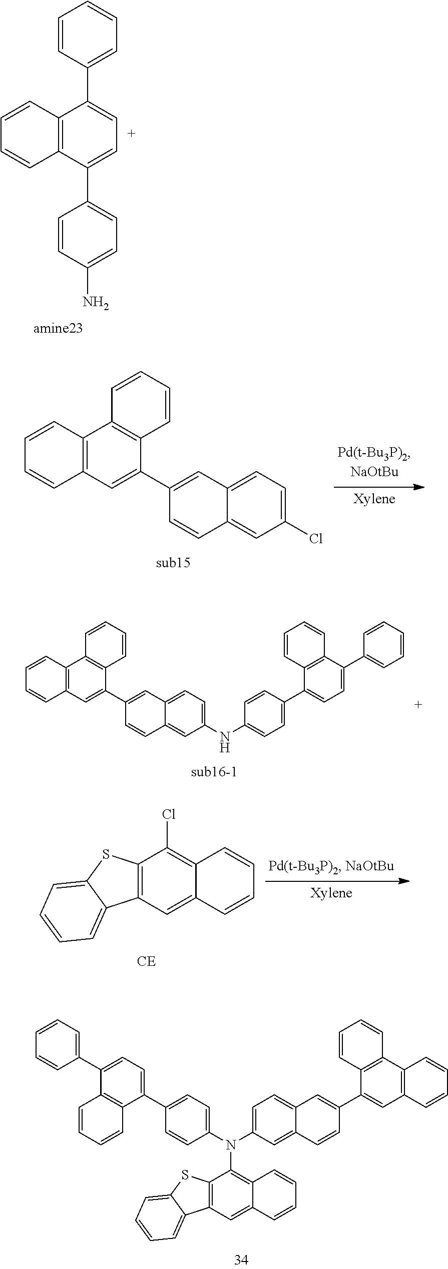 [SMILES: Nc1ccc(-c2ccc(-c3ccccc3)c3ccccc23)cc1 ; Clc1ccc2cc(-c3cc4ccccc4c4ccccc34)ccc2c1 ; c1ccc(-c2ccc(-c3ccc(Nc4ccc5cc(-c6cc7ccccc7c7ccccc67)ccc5c4)cc3)c3ccccc23)cc1 ; Clc1c2ccccc2cc2c1sc1ccccc12 ; c1ccc(-c2ccc(-c3ccc(N(c4ccc5cc(-c6cc7ccccc7c7ccccc67)ccc5c4)c4c5ccccc5cc5c4sc4ccccc45)cc3)c3ccccc23)cc1]

Compound amine23 (10 g, 33.9 mmol), Compound sub15 (11.5 g, 33.9 mmol) and sodium tert-butoxide (4.2 g, 44 mmol) were added to xylene (200 ml) under a nitrogen atmosphere, and the mixture was stirred and refluxed. Then, bis(tri-tert-butylphosphine)palladium(0) (0.3 g, 0.7 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.3 g of Compound sub15-1. (Yield: 66%, MS: [M+H]⁺=598)

Compound sub15-1 (10 g, 16.7 mmol), Compound CE (4.5 g, 16.7 mmol mmol) and sodium tert-butoxide (10.7 g, 50.2 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.7 g of Compound 34. (Yield: 63%, MS: [M+H]⁺=830)

### Example 35

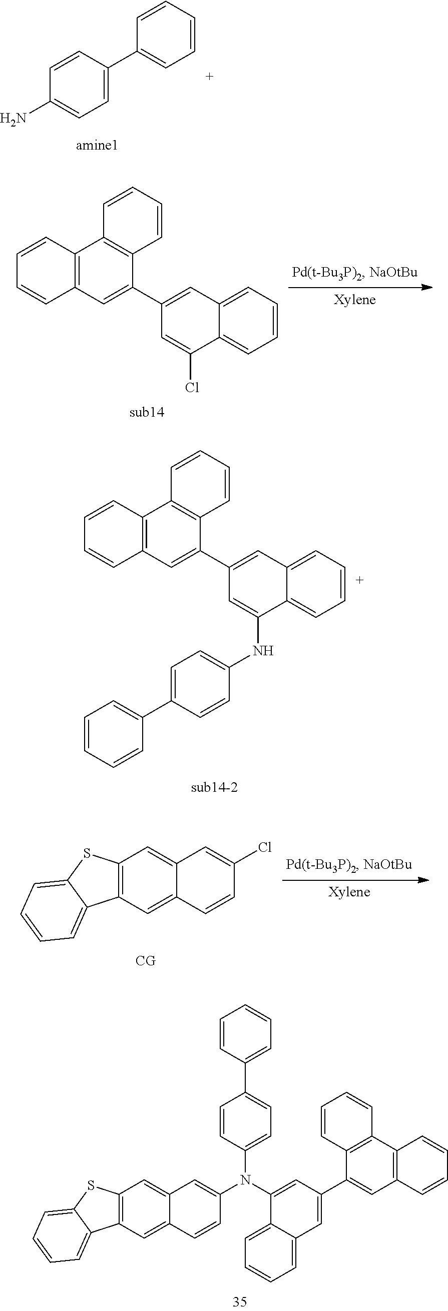 [SMILES: Nc1ccc(-c2ccccc2)cc1 ; Clc1cc(-c2cc3ccccc3c3ccccc23)cc2ccccc12 ; c1ccc(-c2ccc(Nc3cc(-c4cc5ccccc5c5ccccc45)cc4ccccc34)cc2)cc1 ; Clc1ccc2cc3c(cc2c1)sc1ccccc13 ; c1ccc(-c2ccc(N(c3ccc4cc5c(cc4c3)sc3ccccc35)c3cc(-c4cc5ccccc5c5ccccc45)cc4ccccc34)cc2)cc1]

Compound amine1 (10 g, 59.1 mmol), Compound sub14 (17.1 g, 59.1 mmol) and sodium tert-butoxide (7.4 g, 76.8 mmol) were added to xylene (200 ml) under a nitrogen atmosphere, and the mixture was stirred and refluxed. Then, bis(tri-tert-butylphosphine)palladium(0) (0.6 g, 1.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 17.3 g of Compound sub14-2. (Yield: 62%, MS: [M+H]⁺=472)

Compound sub14-2 (10 g, 21.2 mmol), Compound CG (5.7 g, 21.2 mmol) and sodium tert-butoxide (13.5 g, 63.6 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.1 g of Compound 35. (Yield: 68%, MS: [M+H]⁺=704)

### Example 36

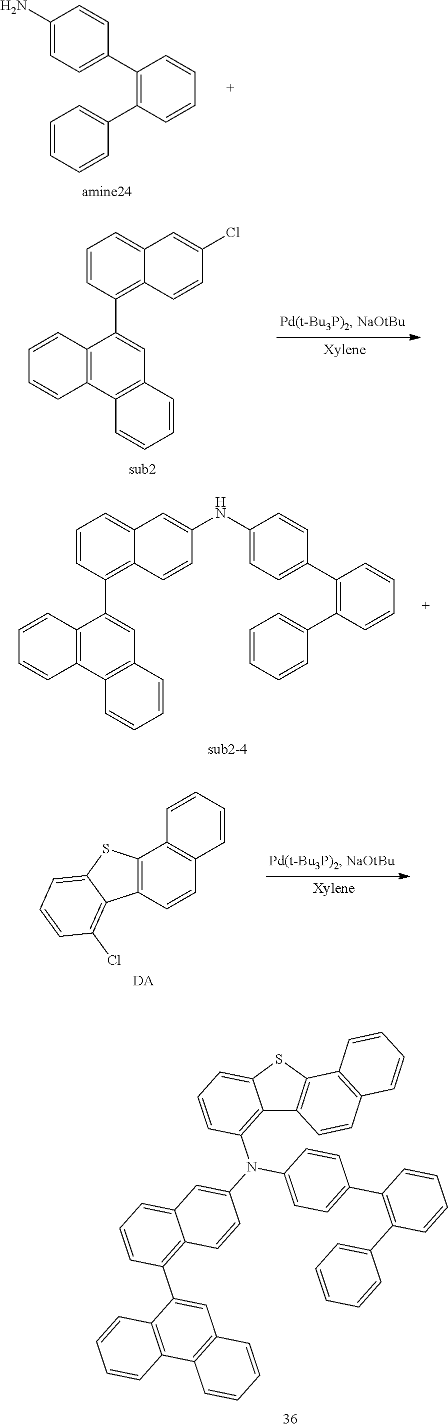

Compound amine24 (10 g, 40.8 mmol), Compound sub2 (13.8 g, 40.8 mmol) and sodium tert-butoxide (5.1 g, 53 mmol) were added to xylene (200 ml) under a nitrogen atmosphere, and the mixture was stirred and refluxed. Then, bis(tri-tert-butylphosphine)palladium(0) (0.4 g, 0.8 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.9 g of Compound sub2-4. (Yield: 67%, MS: [M+H]⁺=548)

Compound sub2-4 (10 g, 18.3 mmol), Compound DA (4.9 g, 18.3 mmol) and sodium tert-butoxide (11.6 g, 54.8 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.5 g of Compound 36. (Yield: 53%, MS: [M+H]⁺=780)

### Example 37

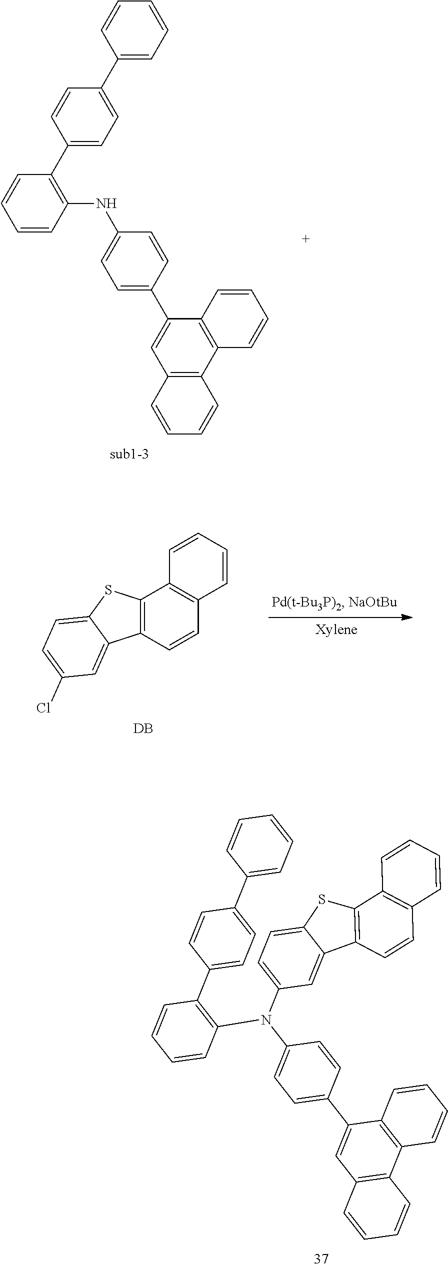 [SMILES: c1ccc(-c2ccc(-c3ccccc3Nc3ccc(-c4cc5ccccc5c5ccccc45)cc3)cc2)cc1 ; Clc1ccc2sc3c4ccccc4ccc3c2c1 ; "DB" ; c1ccc(-c2ccc(-c3ccccc3N(c3ccc(-c4cc5ccccc5c5ccccc45)cc3)c3ccc4sc5c6ccccc6ccc5c4c3)cc2)cc1]

Compound sub1-3 (10 g, 20.1 mmol), Compound DB (5.4 g, 20.1 mmol) and sodium tert-butoxide (12.8 g, 60.3 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.5 g of Compound 37. (Yield: 58%, MS: [M+H]⁺=730)

### Example 38

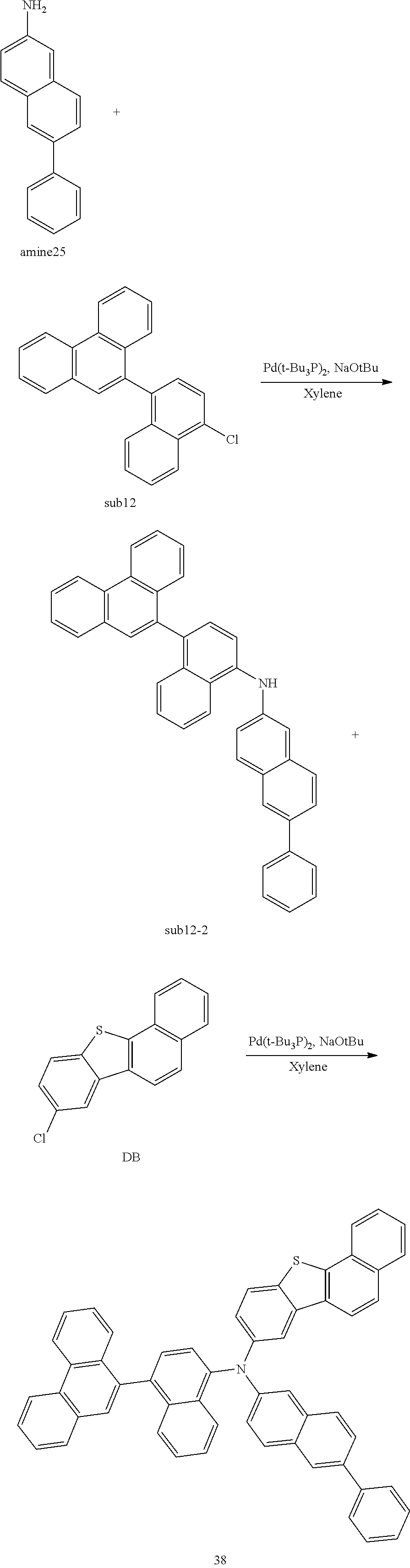 [SMILES: Nc1ccc2cc(-c3ccccc3)ccc2c1 ; Clc1ccc(-c2cc3ccccc3c3ccccc23)c2ccccc12 ; c1ccc(-c2ccc3cc(Nc4ccc(-c5cc6ccccc6c6ccccc56)c5ccccc45)ccc3c2)cc1 ; Clc1ccc2sc3c4ccccc4ccc3c2c1 ; "DB" ; c1ccc(-c2ccc3cc(N(c4ccc5sc6c7ccccc7ccc6c5c4)c4ccc(-c5cc6ccccc6c6ccccc56)c5ccccc45)ccc3c2)cc1]

Compound amine25 (10 g, 45.6 mmol), Compound sub12 (15.5 g, 45.6 mmol) and sodium tert-butoxide (5.7 g, 59.3 mmol) were added to xylene (200 ml) under a nitrogen atmosphere, and the mixture was stirred and refluxed. Then, bis(tri-tert-butylphosphine)palladium(0) (0.5 g, 0.9 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.9 g of Compound sub12-2. (Yield: 50%, MS: [M+H]⁺=522)

Compound sub12-2 (10 g, 19.2 mmol), Compound DB (5.2 g, 19.2 mmol) and sodium tert-butoxide (12.2 g, 57.5 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.8 g of Compound 38. (Yield: 61%, MS: [M+H]⁺=754)

### Example 39

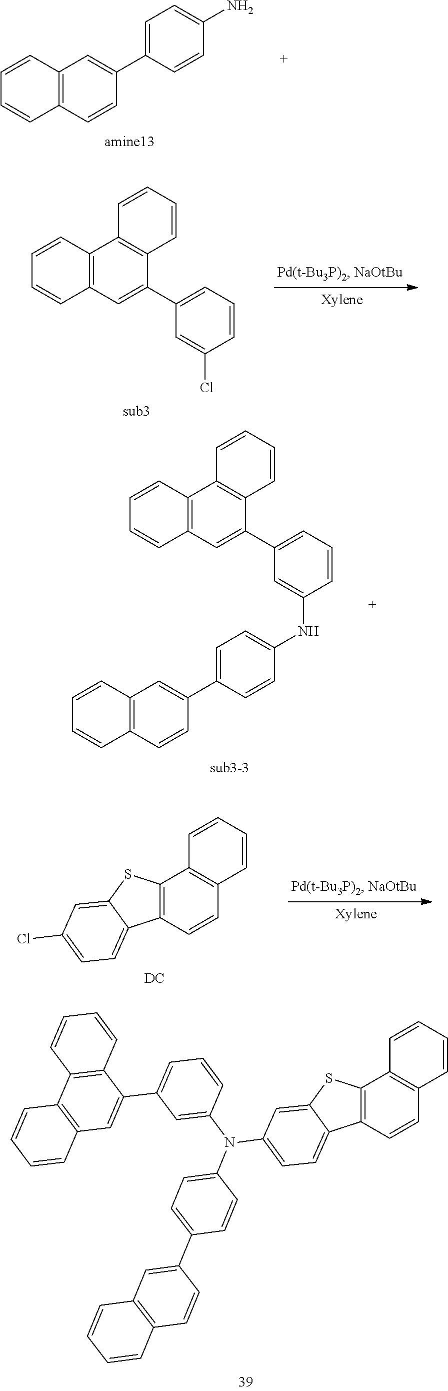

Compound amine13 (10 g, 45.6 mmol), Compound sub3 (13.2 g, 45.6 mmol) and sodium tert-butoxide (5.7 g, 59.3 mmol) were added to xylene (200 ml) under a nitrogen atmosphere, and the mixture was stirred and refluxed. Then, bis(tri-tert-butylphosphine)palladium(0) (0.5 g, 0.9 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.8 g of Compound sub3-3. (Yield: 64%, MS: [M+H]⁺=472)

Compound sub3-3 (10 g, 21.2 mmol), Compound DC (5.7 g, 21.2 mmol) and sodium tert-butoxide (13.5 g, 63.6 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.9 g of Compound 39. (Yield: 60%, MS: [M+H]⁺=704)

### Example 40

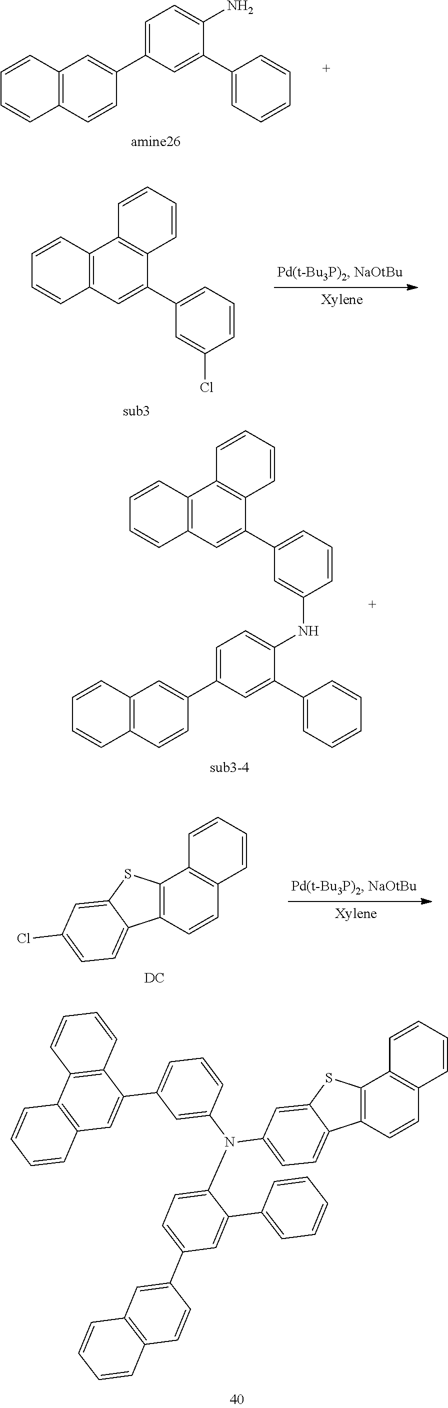

Compound amine26 (10 g, 33.9 mmol), Compound sub3 (9.8 g, 33.9 mmol) and sodium tert-butoxide (4.2 g, 44 mmol) were added to xylene (200 ml) under a nitrogen atmosphere, and the mixture was stirred and refluxed. Then, bis(tri-tert-butylphosphine)palladium(0) (0.3 g, 0.7 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.4 g of Compound sub3-4. (Yield: 67%, MS: [M+H]⁺=548)

Compound sub3-4 (10 g, 18.3 mmol), Compound DC (4.9 g, 18.3 mmol) and sodium tert-butoxide (11.6 g, 54.8 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.7 g of Compound 40. (Yield: 68%, MS: [M+H]⁺=780)

### Example 41

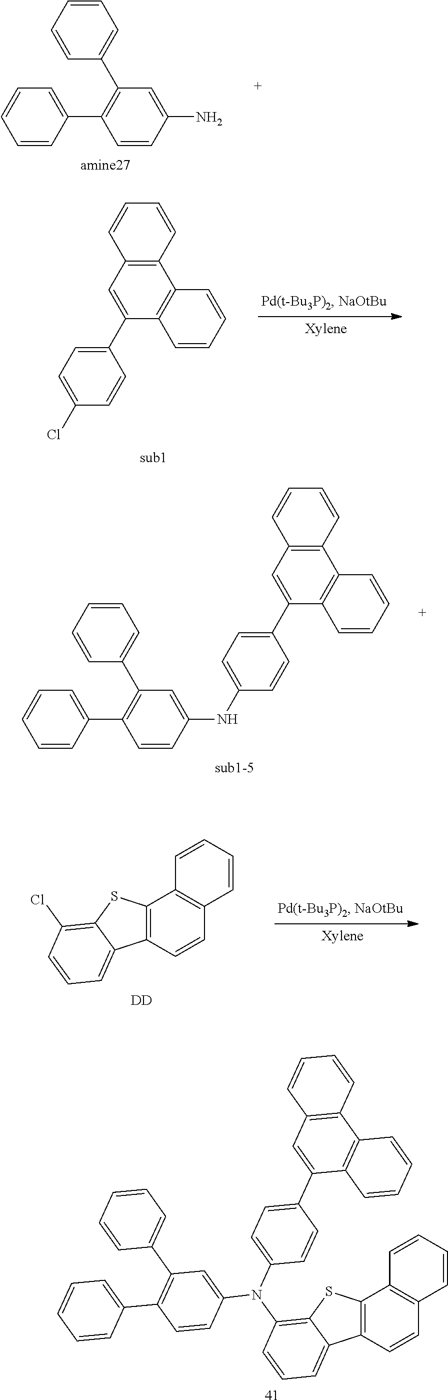

Compound amine27 (10 g, 40.8 mmol), Compound sub1 (11.8 g, 40.8 mmol) and sodium tert-butoxide (5.1 g, 53 mmol) were added to xylene (200 ml) under a nitrogen atmosphere, and the mixture was stirred and refluxed. Then, bis(tri-tert-butylphosphine)palladium(0) (0.4 g, 0.8 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.2 g of Compound sub1-5. (Yield: 65%, MS: [M+H]⁺=498)

Compound sub1-5 (10 g, 20.1 mmol), Compound DC (5.4 g, 20.1 mmol) and sodium tert-butoxide (12.8 g, 60.3 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.4 g of Compound 41. (Yield: 57%, MS: [M+H]⁺=730)

### Example 42

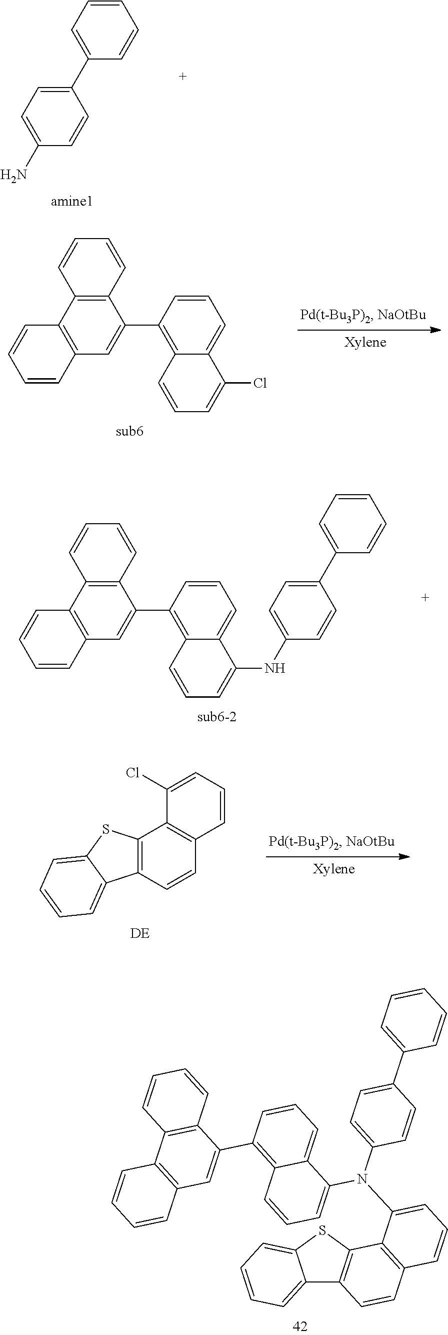 [SMILES: Nc1ccc(-c2ccccc2)cc1 ; Clc1cccc2c(-c3cc4ccccc4c4ccccc34)cccc12 ; c1ccc(-c2ccc(Nc3cccc4c(-c5cc6ccccc6c6ccccc56)cccc34)cc2)cc1 ; Clc1cccc2ccc3c4ccccc4sc3c12 ; "DE" ; c1ccc(-c2ccc(N(c3cccc4c(-c5cc6ccccc6c6ccccc56)cccc34)c3cccc4ccc5c6ccccc6sc5c34)cc2)cc1]

Compound amine1 (10 g, 59.1 mmol), Compound sub6 (20 g, 59.1 mmol) and sodium tert-butoxide (7.4 g, 76.8 mmol) were added to xylene (200 ml) under a nitrogen atmosphere, and the mixture was stirred and refluxed. Then, bis(tri-tert-butylphosphine)palladium(0) (0.6 g, 1.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.2 g of Compound sub6-2. (Yield: 51%, MS: [M+H]⁺=472)

Compound sub6-2 (10 g, 21.2 mmol), Compound DE (5.7 g, 21.2 mmol) and sodium tert-butoxide (13.5 g, 63.6 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.2 g of Compound 42. (Yield: 55%, MS: [M+H]⁺=704)

### Example 43

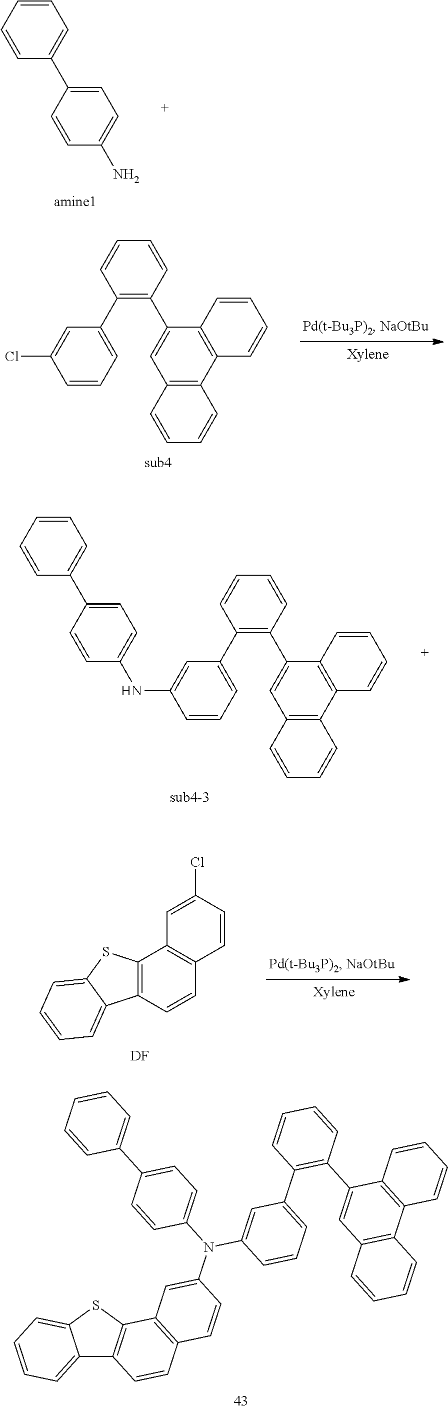

Compound amine1 (10 g, 59.1 mmol), Compound sub4 (21.6 g, 59.1 mmol) and sodium tert-butoxide (7.4 g, 76.8 mmol) were added to xylene (200 ml) under a nitrogen atmosphere, and the mixture was stirred and refluxed. Then, bis(tri-tert-butylphosphine)palladium(0) (0.6 g, 1.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 18.5 g of Compound sub4-3. (Yield: 63%, MS: [M+H]⁺=498)

Compound sub4-3 (10 g, 20.1 mmol), Compound DF (5.4 g, 20.1 mmol) and sodium tert-butoxide (12.8 g, 60.3 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.9 g of Compound 43. (Yield: 54%, MS: [M+H]⁺=730)

### Example 44

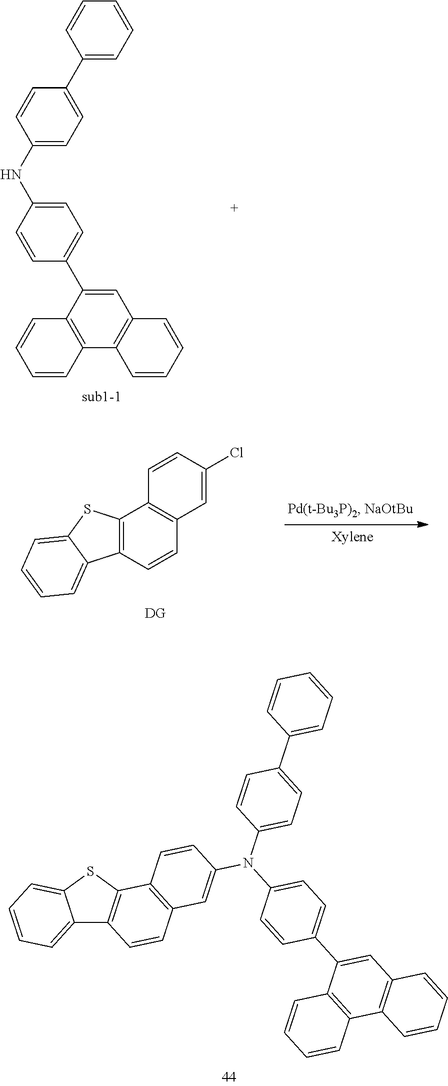

Compound sub1-1 (10 g, 23.7 mmol), Compound DG (6.4 g, 23.7 mmol) and sodium tert-butoxide (15.1 g, 71.2 mmol) were added to xylene (200 ml) under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.3 g of Compound 44. (Yield: 60%, MS: [M+H]⁺=654)

### Example 45

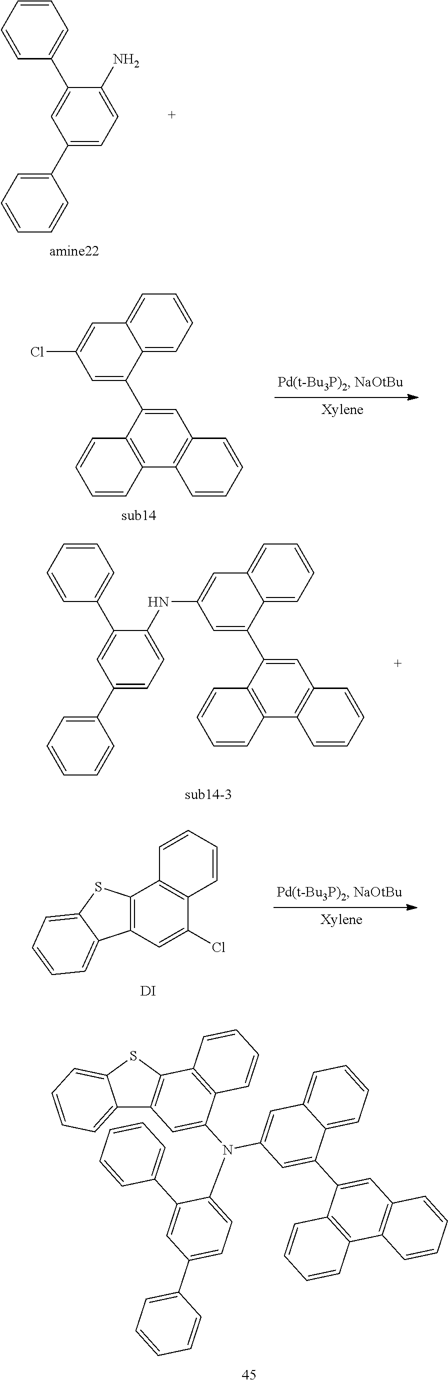 [SMILES: Nc1ccc(-c2ccccc2)cc1-c1ccccc1 ; Clc1cc(-c2cc3ccccc3c3ccccc23)c2ccccc2c1 ; c1ccc(-c2ccc(Nc3cc(-c4cc5ccccc5c5ccccc45)c4ccccc4c3)c(-c3ccccc3)c2)cc1 ; Clc1cc2c3ccccc3sc2c2ccccc12 ; "DI" ; c1ccc(-c2ccc(N(c3cc(-c4cc5ccccc5c5ccccc45)c4ccccc4c3)c3cc4c5ccccc5sc4c4ccccc34)c(-c3ccccc3)c2)cc1]

Compound amine22 (10 g, 40.8 mmol), Compound sub14 (13.8 g, 40.8 mmol) and sodium tert-butoxide (5.1 g, 53 mmol) were added to xylene (200 ml) under a nitrogen atmosphere, and the mixture was stirred and refluxed. Then, bis(tri-tert-butylphosphine)palladium(0) (0.4 g, 0.8 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.4 g of Compound sub14-3. (Yield: 69%, MS: [M+H]⁺=548)

Compound sub14-3 (10 g, 18.3 mmol), Compound DI (4.9 g, 18.3 mmol) and sodium tert-butoxide (11.6 g, 54.8 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.5 g of Compound 45. (Yield: 60%, MS: [M+H]⁺=780)

### Example 46

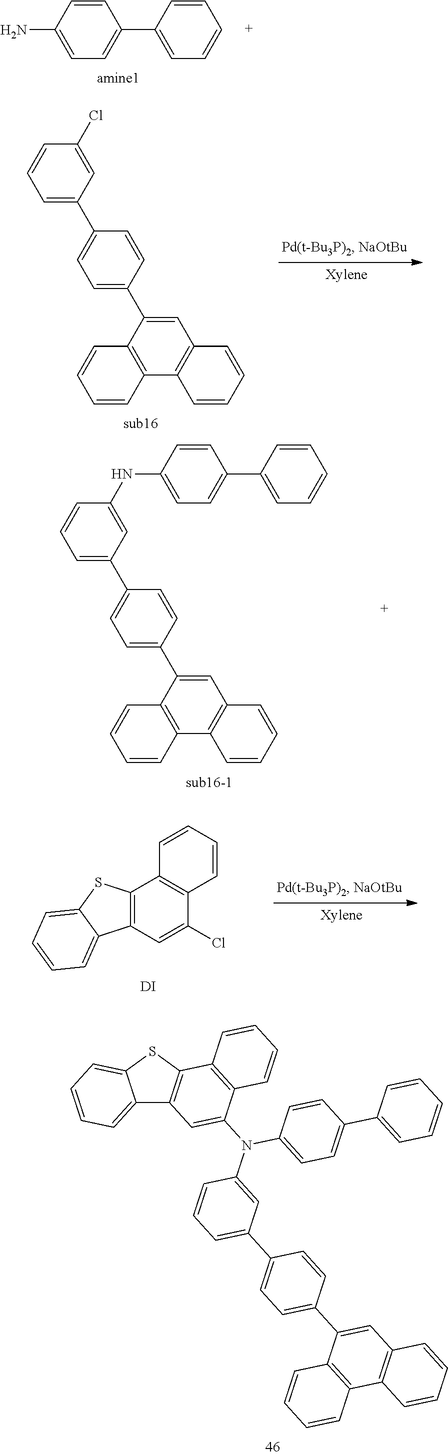

Compound amine1 (10 g, 59.1 mmol), Compound sub16 (21.6 g, 59.1 mmol) and sodium tert-butoxide (7.4 g, 76.8 mmol) were added to xylene (200 ml) under a nitrogen atmosphere, and the mixture was stirred and refluxed. Then, bis(tri-tert-butylphosphine)palladium(0) (0.6 g, 1.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.5 g of Compound sub16-1. (Yield: 56%, MS: [M+H]⁺=498)

Compound sub16-1 (10 g, 20.1 mmol), Compound DI (5.4 g, 20.1 mmol) and sodium tert-butoxide (12.8 g, 60.3 mmol) were added to xylene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.9 g of Compound 46. (Yield: 61%, MS: [M+H]⁺=730)

### Example 47

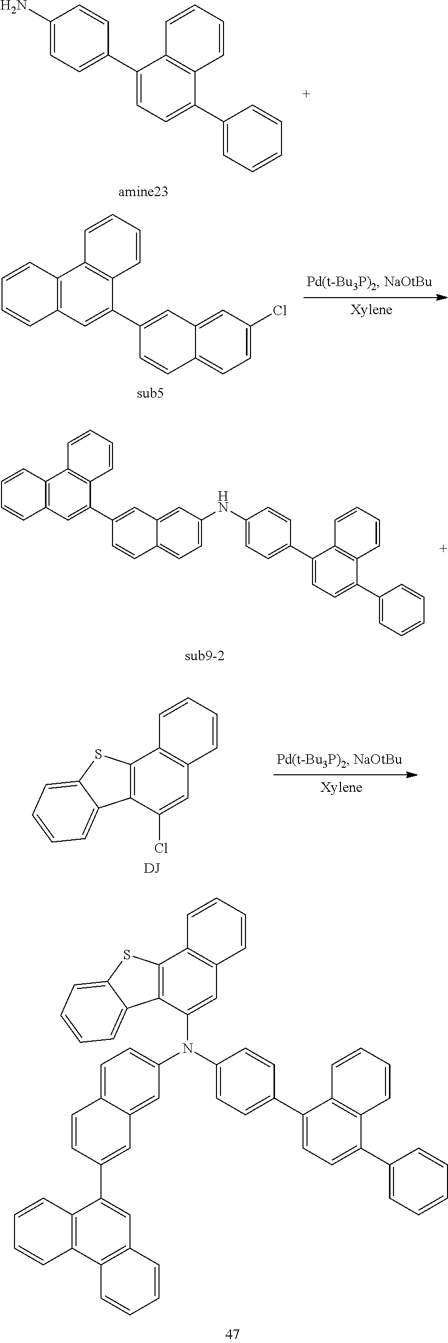

Compound sub9-2 (10 g, 16.7 mmol), Compound DJ (4.5 g, 16.7 mmol) and sodium tert-butoxide (10.7 g, 50.2 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.8 g of Compound 47. (Yield: 56%, MS: [M+H]⁺=830)

### EXPERIMENTAL EXAMPLES

### Experimental Example 1

A glass substrate on which a thin film of ITO (indium tin oxide) was coated in a thickness of 1,000 Å was put into distilled water containing a detergent dissolved therein and washed by the ultrasonic waves. In this case, the detergent used was a product commercially available from Fisher Co. and the distilled water was one which had been twice filtered by using a filter commercially available from Millipore Co. The ITO was washed for 30 minutes, and ultrasonic washing was then repeated twice for 10 minutes by using distilled water. After the washing with distilled water was completed, the substrate was ultrasonically washed with isopropyl alcohol, acetone, and methanol solvent, and dried, after which it was transported to a plasma cleaner. Then, the substrate was cleaned with oxygen plasma for 5 minutes, and then transferred to a vacuum evaporator.

On the ITO transparent electrode thus prepared, the following Compound HI-1 was formed to a thickness of 1150 Å as a hole injection layer, but the following Compound A-1 was p-doped at a concentration of 1.5 wt. %. The following Compound HT-1 was vacuum deposited on the hole injection layer to form a hole transport layer with a film thickness of 800 Å. Then, the following Compound EB-1 was vacuum deposited on the hole transport layer to form an electron blocking layer with a film thickness of 150 Å. The Compound 1 previously prepared, the following Compound RH-1, and the following Compound Dp-7 were vacuum deposited in a weight ratio of 49:49:2 on the electron blocking layer to form a light emitting layer with a thickness of 400 Å. The following Compound HB-1 was vacuum deposited on the light emitting layer to form a hole blocking layer with a film thickness of 30 Å. The following Compound ET-1 and the following Compound LiQ were vacuum deposited in a ratio of 2:1 on the hole blocking layer to form an electron injection and transport layer with a film thickness of 300 Å. Lithium fluoride (LiF) and aluminum were sequentially deposited to have a thickness of 12 Å and 1,000 Å, respectively, on the electron injection and transport layer, thereby forming a cathode.

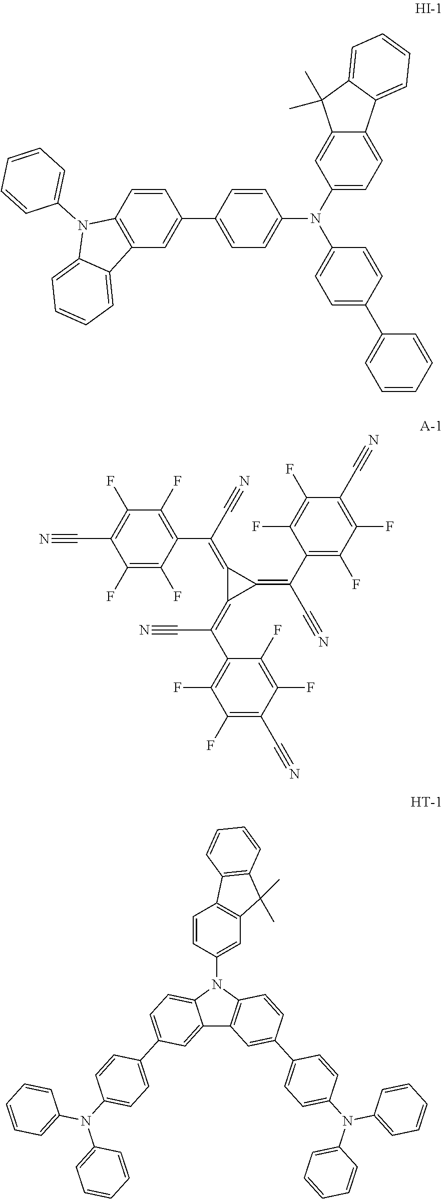 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; CC1(C)c2ccccc2-c2ccc(-n3c4ccc(-c5ccc(N(c6ccccc6)c6ccccc6)cc5)cc4c4cc(-c5ccc(N(c6ccccc6)c6ccccc6)cc5)ccc43)cc21]

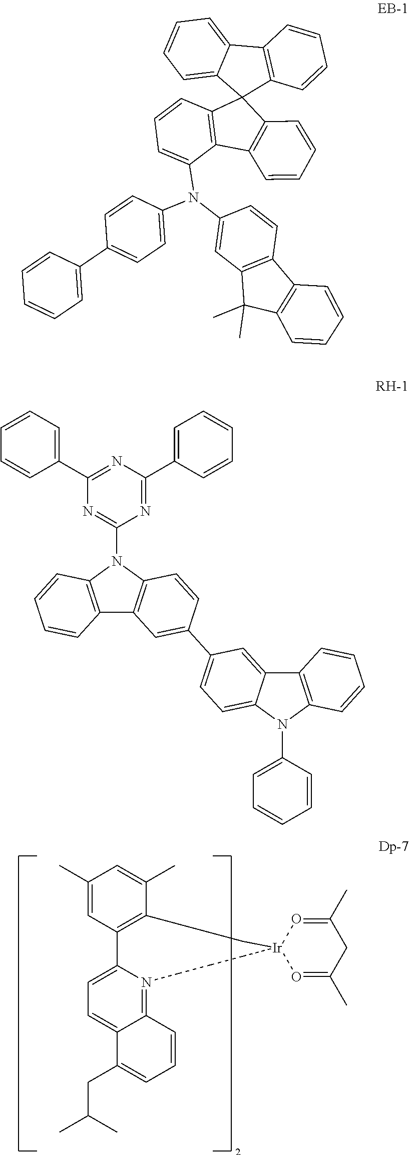

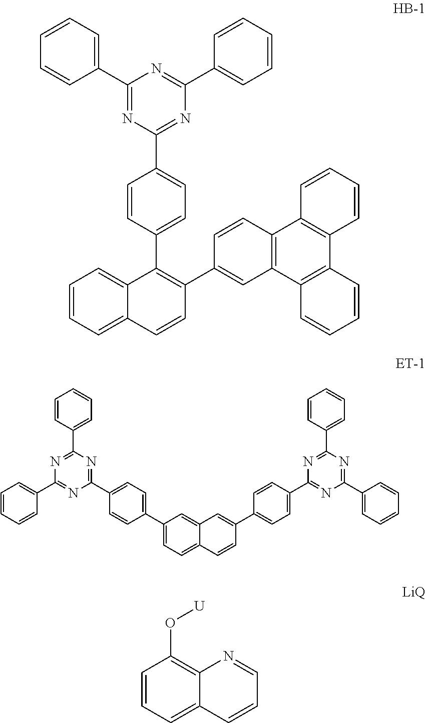

In the above-mentioned processes, the deposition rates of the organic materials were maintained at 0.4˜0.7 Å/sec, the deposition rates of lithium fluoride and the aluminum of the cathode were maintained at 0.3 Å/sec and 2 Å/sec, respectively, and the degree of vacuum during the deposition was maintained at 2×10⁻⁷˜5×10⁻⁶ torr, thereby manufacturing an organic light emitting device.

### Experimental Examples 2 to 47

The organic light emitting devices were manufactured in the same manner as in Experimental Example 1, except that the compounds shown in Tables 1 and 2 below were used instead of Compound 1.

### Comparative Experimental Examples 1 to 10

The organic light emitting devices were manufactured in the same manner as in Experimental Example 1, except that the compounds shown in Table 3 below were used instead of Compound 1. In Table 3, the Compounds C-1 to C-10 are as follows:

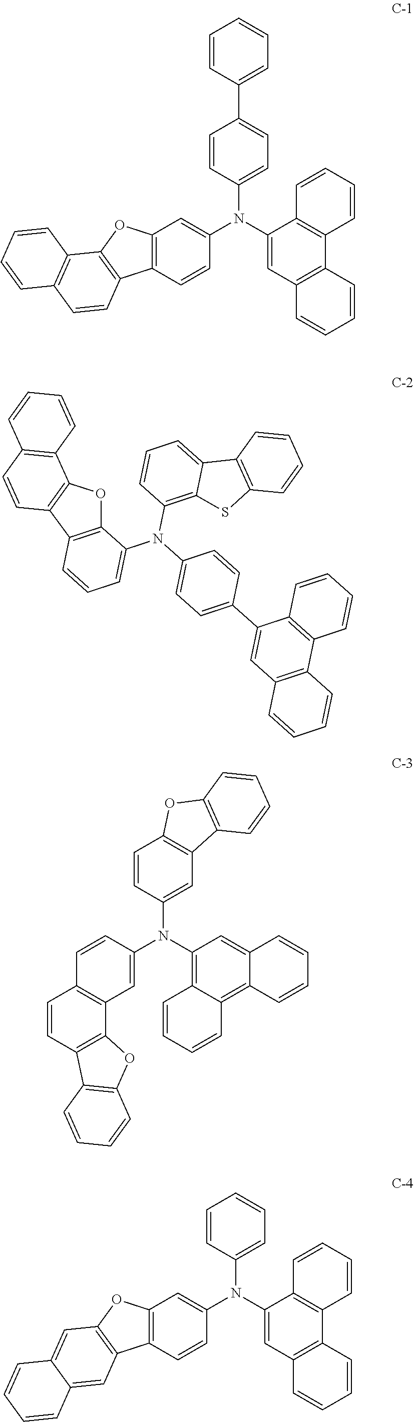 [SMILES: c1ccc(-c2ccc(N(c3ccc4c(c3)oc3c5ccccc5ccc43)c3cc4ccccc4c4ccccc34)cc2)cc1 ; c1ccc2c(c1)cc(-c1ccc(N(c3cccc4c3oc3c5ccccc5ccc43)c3cccc4c3sc3ccccc34)cc1)c1ccccc12 ; c1ccc2c(c1)cc(N(c1ccc3oc4ccccc4c3c1)c1ccc3ccc4c5ccccc5oc4c3c1)c1ccccc12 ; c1ccc(N(c2ccc3c(c2)oc2cc4ccccc4cc23)c2cc3ccccc3c3ccccc23)cc1]

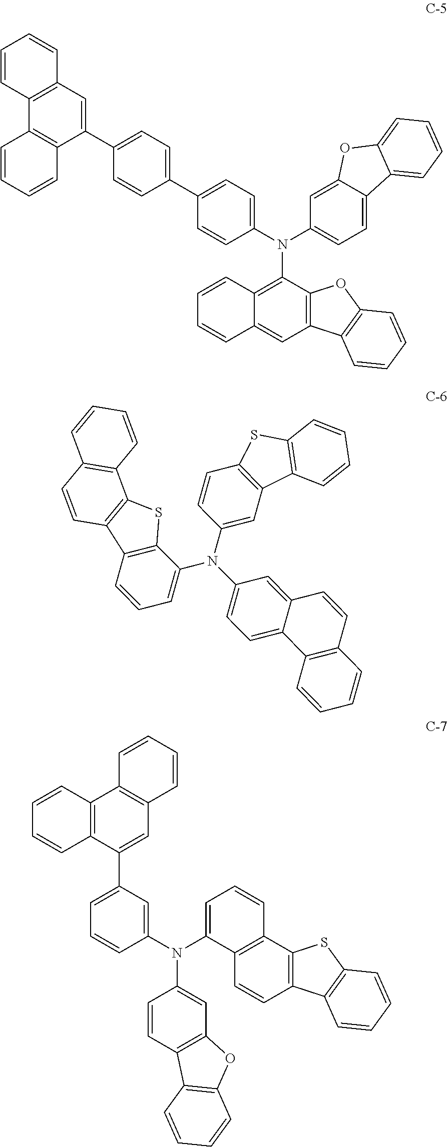 [SMILES: c1ccc2c(N(c3ccc(-c4ccc(-c5cc6ccccc6c6ccccc56)cc4)cc3)c3ccc4c(c3)oc3ccccc34)c3oc4ccccc4c3cc2c1 ; c1ccc2c(c1)ccc1cc(N(c3ccc4sc5ccccc5c4c3)c3cccc4c3sc3c5ccccc5ccc43)ccc12 ; c1cc(-c2cc3ccccc3c3ccccc23)cc(N(c2ccc3c(c2)oc2ccccc23)c2cccc3c2ccc2c4ccccc4sc32)c1]

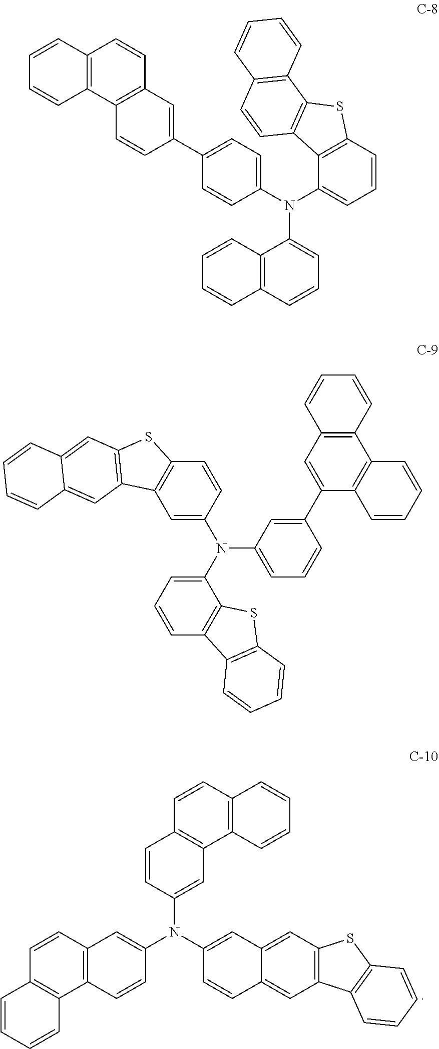 [SMILES: c1ccc2c(N(c3ccc(-c4ccc5c(ccc6ccccc65)c4)cc3)c3cccc4sc5c6ccccc6ccc5c34)cccc2c1 ; c1cc(-c2cc3ccccc3c3ccccc23)cc(N(c2ccc3sc4cc5ccccc5cc4c3c2)c2cccc3c2sc2ccccc23)c1 ; c1ccc2c(c1)ccc1cc(N(c3ccc4cc5c(cc4c3)sc3ccccc35)c3ccc4ccc5ccccc5c4c3)ccc12]

The driving voltage and luminous efficiency were measured (15 mA/cm²) by applying a current to the organic light emitting devices manufactured in the Experimental Examples and Comparative Experimental Examples, and the results are shown in Tables 1 to 3 below. Lifetime T95 means the time required for the luminance to be reduced to 95% of the initial luminance (6000 nit).

TABLE 1

| | | Driving | Luminous | Lifetime | |
|---|---|---|---|---|---|
| | Compound | voltage | efficiency | T95 | Luminous |
| | (host) | (V) | (cd/A) | (hr) | color |
| Experimental | Compound 1 | 4.00 | 16.24 | 154 | Red |
| Example 1 | | | | | |
| Experimental | Compound 2 | 4.38 | 17.93 | 150 | Red |
| Example 2 | | | | | |
| Experimental | Compound 3 | 4.45 | 17.78 | 168 | Red |
| Example 3 | | | | | |
| Experimental | Compound 4 | 4.34 | 17.52 | 174 | Red |
| Example 4 | | | | | |
| Experimental | Compound 5 | 4.38 | 16.34 | 179 | Red |
| Example 5 | | | | | |
| Experimental | Compound 6 | 4.36 | 17.74 | 179 | Red |
| Example 6 | | | | | |
| Experimental | Compound 7 | 4.27 | 17.52 | 169 | Red |
| Example 7 | | | | | |
| Experimental | Compound 8 | 4.38 | 16.02 | 163 | Red |
| Example 8 | | | | | |
| Experimental | Compound 9 | 4.32 | 16.24 | 156 | Red |
| Example 9 | | | | | |
| Experimental | Compound 10 | 4.40 | 16.26 | 173 | Red |
| Example 10 | | | | | |
| Experimental | Compound 11 | 4.39 | 17.28 | 180 | Red |
| Example 11 | | | | | |
| Experimental | Compound 12 | 4.42 | 16.85 | 156 | Red |
| Example 12 | | | | | |
| Experimental | Compound 13 | 4.29 | 16.99 | 133 | Red |
| Example 13 | | | | | |
| Experimental | Compound 14 | 4.39 | 17.28 | 160 | Red |
| Example 14 | | | | | |
| Experimental | Compound 15 | 4.42 | 16.85 | 133 | Red |
| Example 15 | | | | | |
| Experimental | Compound 16 | 4.23 | 17.80 | 154 | Red |
| Example 16 | | | | | |
| Experimental | Compound 17 | 4.25 | 16.70 | 131 | Red |
| Example 17 | | | | | |
| Experimental | Compound 18 | 4.36 | 16.70 | 137 | Red |
| Example 18 | | | | | |
| Experimental | Compound 19 | 4.33 | 16.42 | 152 | Red |
| Example 19 | | | | | |
| Experimental | Compound 20 | 4.43 | 17.12 | 131 | Red |
| Example 20 | | | | | |
| Experimental | Compound 21 | 4.37 | 17.18 | 142 | Red |
| Example 21 | | | | | |
| Experimental | Compound 22 | 4.17 | 17.07 | 136 | Red |
| Example 22 | | | | | |
| Experimental | Compound 23 | 4.21 | 17.87 | 137 | Red |
| Example 23 | | | | | |
| Experimental | Compound 24 | 4.23 | 16.15 | 138 | Red |
| Example 24 | | | | | |
| Experimental | Compound 25 | 4.32 | 16.60 | 156 | Red |
| Example 25 | | | | | |

TABLE 2

| | | Driving | Luminous | Lifetime | |
|---|---|---|---|---|---|
| | Compound | voltage | efficiency | T95 | Luminous |
| | (host) | (V) | (cd/A) | (hr) | color |
| Experimental | Compound 26 | 4.36 | 17.65 | 131 | Red |
| Example 26 | | | | | |
| Experimental | Compound 27 | 4.33 | 17.54 | 149 | Red |
| Example 27 | | | | | |
| Experimental | Compound 28 | 4.32 | 17.39 | 147 | Red |
| Example 28 | | | | | |
| Experimental | Compound 29 | 4.26 | 17.90 | 141 | Red |
| Example 29 | | | | | |
| Experimental | Compound 30 | 4.31 | 17.64 | 135 | Red |
| Example 30 | | | | | |
| Experimental | Compound 31 | 4.35 | 17.43 | 139 | Red |
| Example 31 | | | | | |
| Experimental | Compound 32 | 4.26 | 17.83 | 123 | Red |
| Example 32 | | | | | |
| Experimental | Compound 33 | 4.29 | 17.77 | 135 | Red |
| Example 33 | | | | | |
| Experimental | Compound 34 | 4.30 | 17.57 | 143 | Red |
| Example 34 | | | | | |
| Experimental | Compound 35 | 4.37 | 17.31 | 133 | Red |
| Example 35 | | | | | |
| Experimental | Compound 36 | 4.16 | 15.53 | 128 | Red |
| Example 36 | | | | | |
| Experimental | Compound 37 | 4.19 | 16.44 | 134 | Red |
| Example 37 | | | | | |
| Experimental | Compound 38 | 4.18 | 15.30 | 126 | Red |
| Example 38 | | | | | |
| Experimental | Compound 39 | 4.12 | 15.91 | 123 | Red |
| Example 39 | | | | | |
| Experimental | Compound 40 | 4.12 | 15.36 | 138 | Red |
| Example 40 | | | | | |
| Experimental | Compound 41 | 4.28 | 16.39 | 138 | Red |
| Example 41 | | | | | |
| Experimental | Compound 42 | 4.21 | 15.46 | 129 | Red |
| Example 42 | | | | | |
| Experimental | Compound 43 | 4.23 | 15.48 | 129 | Red |
| Example 43 | | | | | |
| Experimental | Compound 44 | 4.16 | 15.31 | 122 | Red |
| Example 44 | | | | | |
| Experimental | Compound 45 | 4.19 | 16.41 | 117 | Red |
| Example 45 | | | | | |
| Experimental | Compound 46 | 4.14 | 15.75 | 124 | Red |
| Example 46 | | | | | |
| Experimental | Compound 47 | 4.29 | 15.26 | 132 | Red |
| Example 47 | | | | | |

TABLE 3

| | | Driving | Luminous | Lifetime | |
|---|---|---|---|---|---|
| | Compound | voltage | efficiency | T95 | Luminous |
| | (host) | (V) | (cd/A) | (hr) | color |
| Comparative | Compound | 4.63 | 13.28 | 96 | Red |
| Experimental | C-1 | | | | |
| Example 1 | | | | | |
| Comparative | Compound | 4.53 | 14.43 | 107 | Red |
| Experimental | C-2 | | | | |
| Example 2 | | | | | |
| Comparative | Compound | 4.70 | 13.46 | 81 | Red |
| Experimental | C-3 | | | | |
| Example 3 | | | | | |
| Comparative | Compound | 4.65 | 13.12 | 87 | Red |
| Experimental | C-4 | | | | |
| Example 4 | | | | | |
| Comparative | Compound | 4.56 | 14.21 | 104 | Red |
| Experimental | C-5 | | | | |
| Example 5 | | | | | |
| Comparative | Compound | 4.69 | 13.72 | 98 | Red |
| Experimental | C-6 | | | | |
| Example 6 | | | | | |
| Comparative | Compound | 4.62 | 14.28 | 106 | Red |
| Experimental | C-7 | | | | |
| Example 7 | | | | | |
| Comparative | Compound | 4.67 | 14.09 | 96 | Red |
| Experimental | C-8 | | | | |
| Example 8 | | | | | |
| Comparative | Compound | 4.54 | 14.24 | 102 | Red |
| Experimental | C-9 | | | | |
| Example 9 | | | | | |
| Comparative | Compound | 4.67 | 13.74 | 87 | Red |
| Experimental | C-10 | | | | |
| Example 10 | | | | | |

As shown in Tables 1 to 3, when the compound of the present disclosure was used as a host for the light emitting layer, the driving voltage was significantly reduced and the efficiency was also significant increased as compared with the materials used in Comparative Experimental Examples, confirming that energy transfer from the host to the red dopant was well performed. In addition, it was confirmed that the lifetime characteristics could be significantly improved while maintaining high efficiency.

It can be judged that this is because the compound of the present disclosure has higher stability to electrons and holes than the compounds used in Comparative Experimental Examples. In conclusion, it can be confirmed that when the compound of the present disclosure was used as a host for the red light emitting layer, the driving voltage, luminous efficiency, and lifetime characteristics of the organic light emitting devices could be improved.

[Description of Symbols]

| | |
|---|---|
| 1: substrate | 2: anode |
| 3: light emitting layer | 4: cathode |
| 5: hole injection layer | 6: hole transport layer |
| 7: light emitting layer | 8: electron transport layer |

1. A compound of Chemical Formula 1: 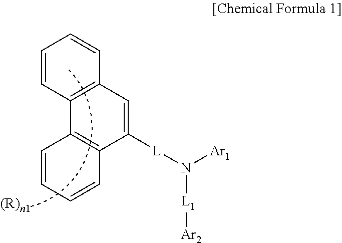
wherein in Chemical Formula 1;
L is a substituted or unsubstituted C₆₋₆₀ arylene;
L₁ is a single bond or a substituted or unsubstituted C₆₋₆₀ arylene;
Ar₁ is any one substituent of the following: 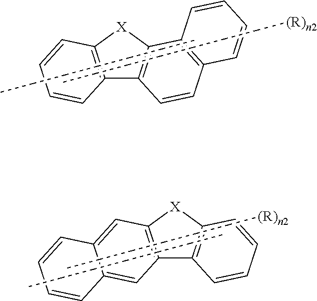
X is O or S;
Ar₂ is a substituted or unsubstituted C₆₋₆₀ aryl;
each R is independently hydrogen or deuterium;
n1 is an integer of 0 to 9; and
n2 is an integer of 0 to 9.

2. The compound of claim 1, wherein L is phenylene, biphenyldiyl, or naphthylene.

3. The compound of claim 1, wherein L is any one selected from the group consisting of:

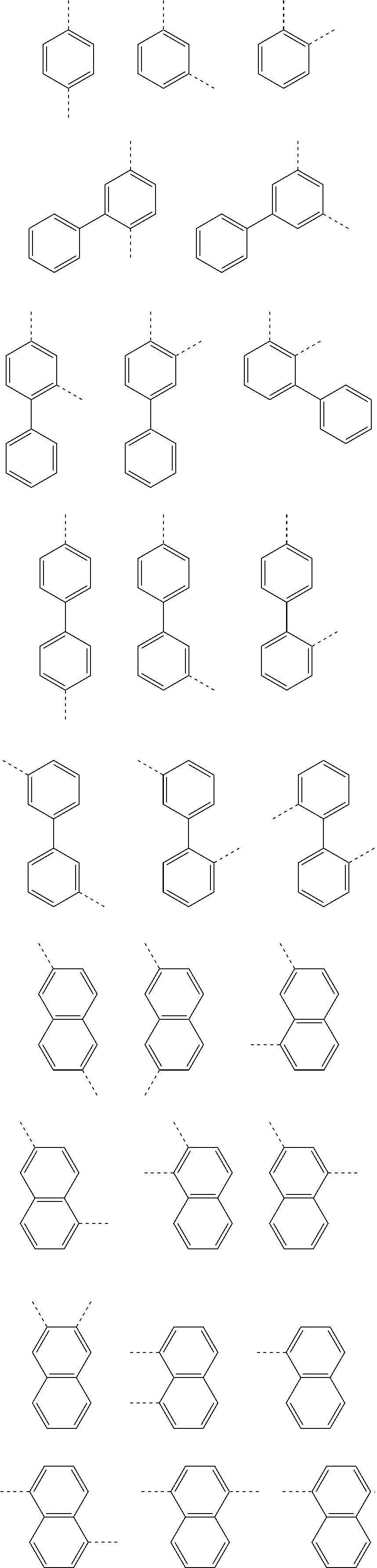

4. The compound of claim 1, wherein L₁ is a single bond, phenylene, biphenyldiyl, or naphthylene.

5. The compound of claim 1, wherein L₁ is a single bond, or any one selected from the group consisting of:

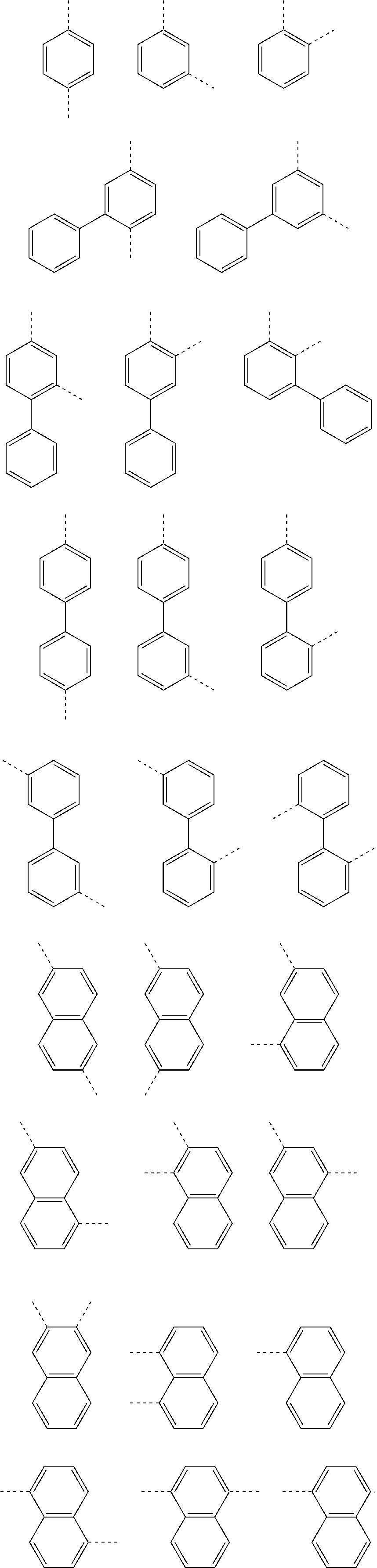

6. The compound of claim 1, wherein Ar₂ is phenyl, biphenylyl, terphenylyl, naphthyl, phenylnaphthyl, naphthylphenyl, phenanthrenyl, or triphenylenyl.

7. The compound of claim 1, wherein the compound of Chemical Formula 1 is any one compound selected from the group consisting of:

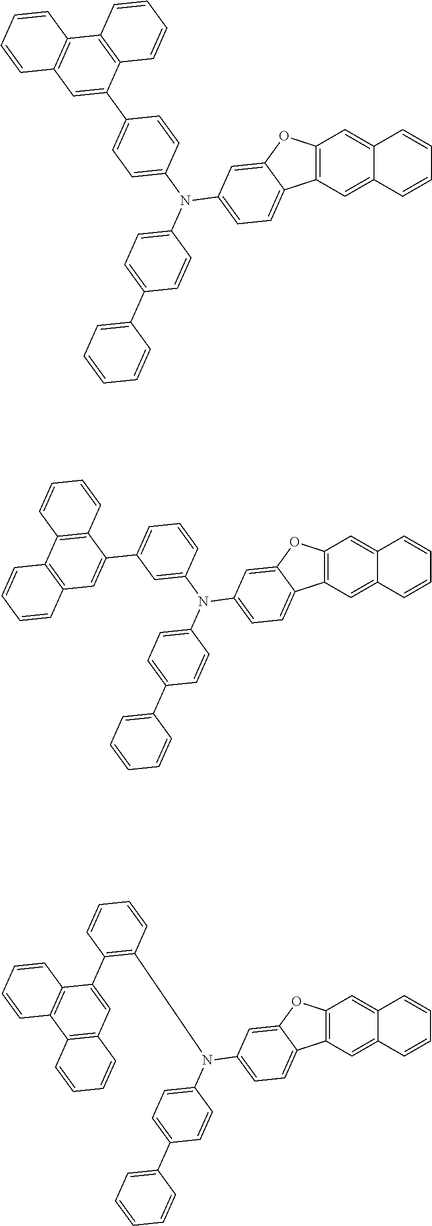 [SMILES: c1ccc(-c2ccc(N(c3ccc(-c4cc5ccccc5c5ccccc45)cc3)c3ccc4c(c3)oc3cc5ccccc5cc34)cc2)cc1 ; c1ccc(-c2ccc(N(c3cccc(-c4cc5ccccc5c5ccccc45)c3)c3ccc4c(c3)oc3cc5ccccc5cc34)cc2)cc1 ; c1ccc(-c2ccc(N(c3ccc4c(c3)oc3cc5ccccc5cc34)c3ccccc3-c3cc4ccccc4c4ccccc34)cc2)cc1]

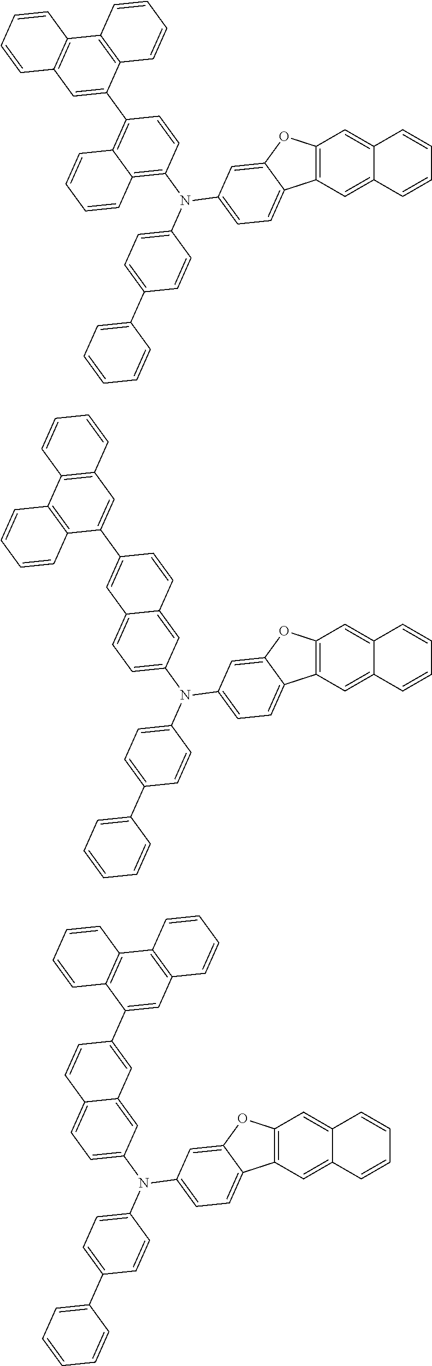

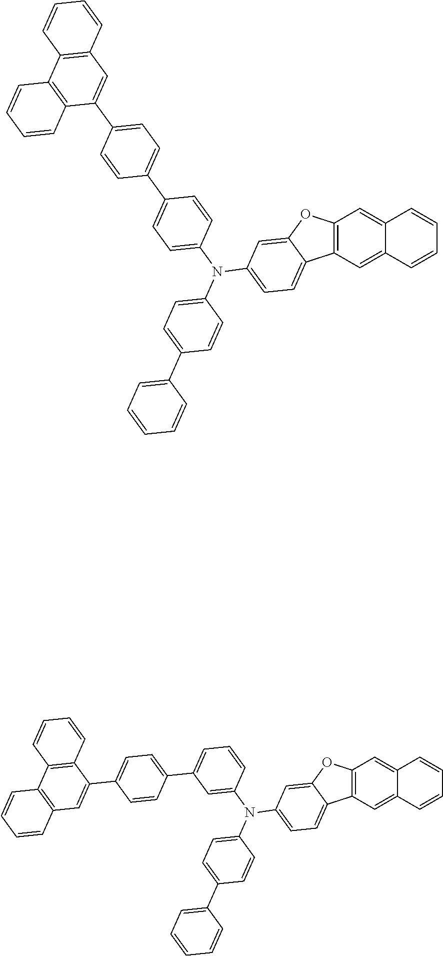 [SMILES: c1ccc(-c2ccc(N(c3ccc(-c4ccc(-c5cc6ccccc6c6ccccc56)cc4)cc3)c3ccc4c(c3)oc3cc5ccccc5cc34)cc2)cc1 ; c1ccc(-c2ccc(N(c3cccc(-c4ccc(-c5cc6ccccc6c6ccccc56)cc4)c3)c3ccc4c(c3)oc3cc5ccccc5cc34)cc2)cc1]

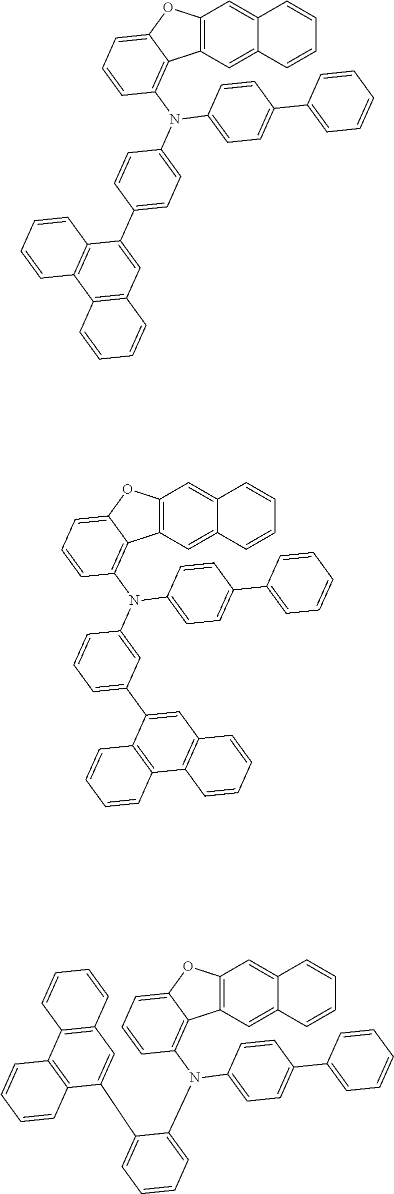 [SMILES: c1ccc(-c2ccc(N(c3ccc(-c4cc5ccccc5c5ccccc45)cc3)c3cccc4oc5cc6ccccc6cc5c34)cc2)cc1 ; c1ccc(-c2ccc(N(c3cccc(-c4cc5ccccc5c5ccccc45)c3)c3cccc4oc5cc6ccccc6cc5c34)cc2)cc1 ; c1ccc(-c2ccc(N(c3ccccc3-c3cc4ccccc4c4ccccc34)c3cccc4oc5cc6ccccc6cc5c34)cc2)cc1]

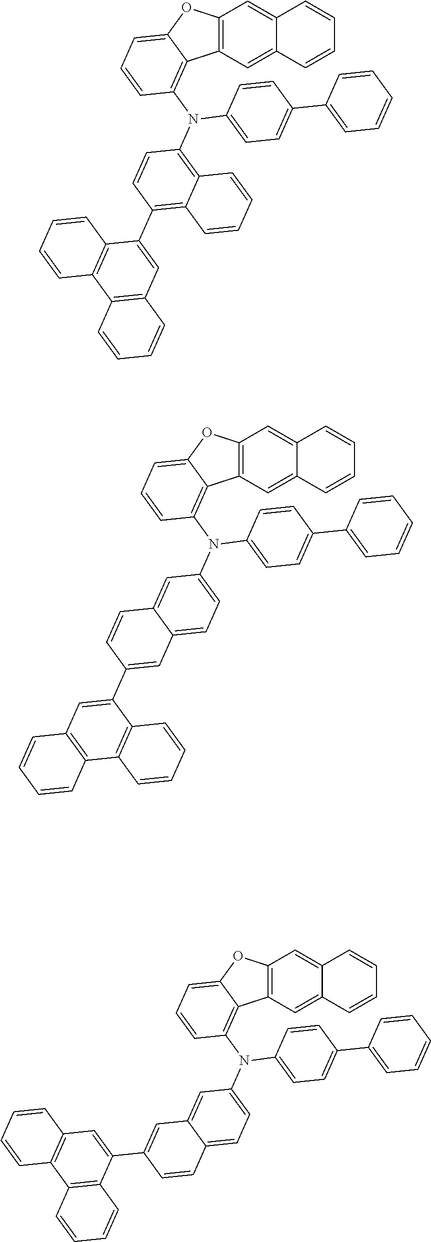 [SMILES: c1ccc(-c2ccc(N(c3ccc(-c4cc5ccccc5c5ccccc45)c4ccccc34)c3cccc4oc5cc6ccccc6cc5c34)cc2)cc1 ; c1ccc(-c2ccc(N(c3ccc4cc(-c5cc6ccccc6c6ccccc56)ccc4c3)c3cccc4oc5cc6ccccc6cc5c34)cc2)cc1 ; c1ccc(-c2ccc(N(c3ccc4ccc(-c5cc6ccccc6c6ccccc56)cc4c3)c3cccc4oc5cc6ccccc6cc5c34)cc2)cc1]

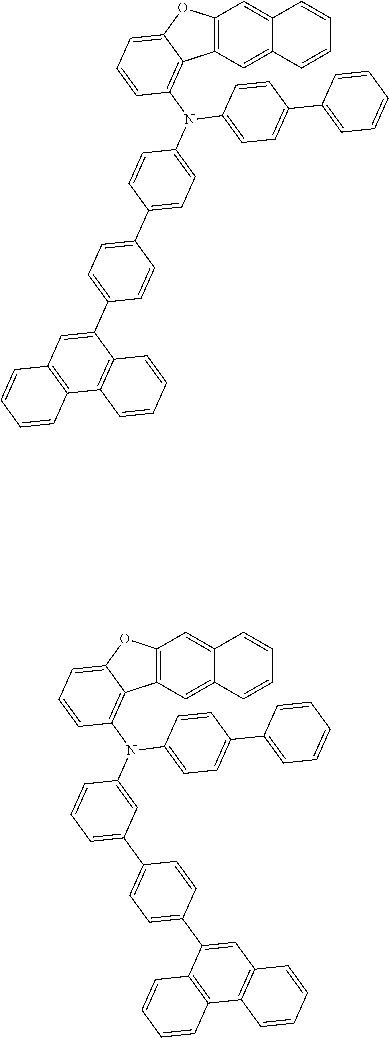 [SMILES: c1ccc(-c2ccc(N(c3ccc(-c4ccc(-c5cc6ccccc6c6ccccc56)cc4)cc3)c3cccc4oc5cc6ccccc6cc5c34)cc2)cc1 ; c1ccc(-c2ccc(N(c3cccc(-c4ccc(-c5cc6ccccc6c6ccccc56)cc4)c3)c3cccc4oc5cc6ccccc6cc5c34)cc2)cc1]

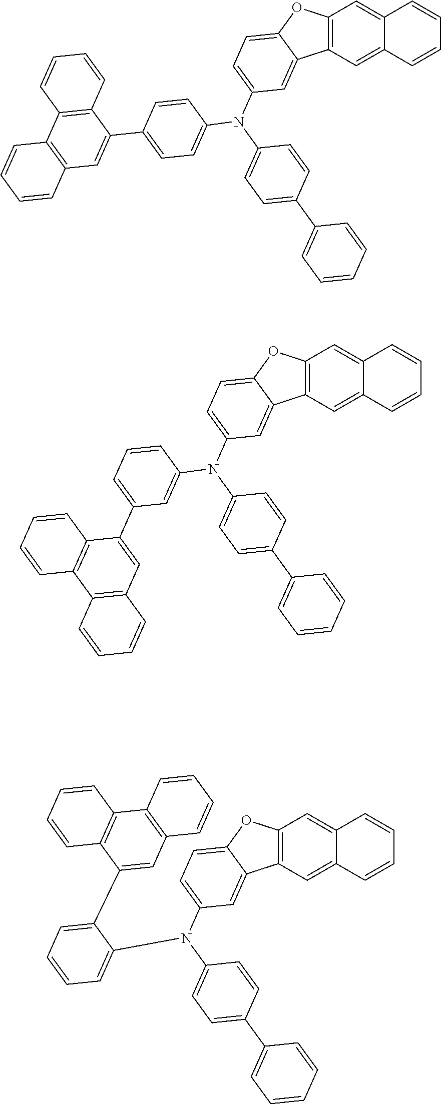 [SMILES: c1ccc(-c2ccc(N(c3ccc(-c4cc5ccccc5c5ccccc45)cc3)c3ccc4oc5cc6ccccc6cc5c4c3)cc2)cc1 ; c1ccc(-c2ccc(N(c3cccc(-c4cc5ccccc5c5ccccc45)c3)c3ccc4oc5cc6ccccc6cc5c4c3)cc2)cc1 ; c1ccc(-c2ccc(N(c3ccc4oc5cc6ccccc6cc5c4c3)c3ccccc3-c3cc4ccccc4c4ccccc34)cc2)cc1]

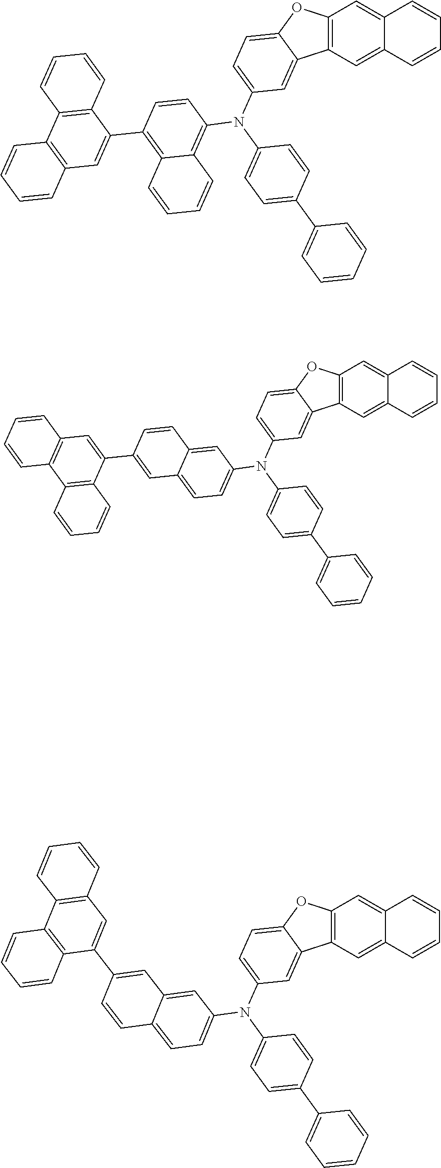 [SMILES: c1ccc(-c2ccc(N(c3ccc4oc5cc6ccccc6cc5c4c3)c3ccc(-c4cc5ccccc5c5ccccc45)c4ccccc34)cc2)cc1 ; c1ccc(-c2ccc(N(c3ccc4cc(-c5cc6ccccc6c6ccccc56)ccc4c3)c3ccc4oc5cc6ccccc6cc5c4c3)cc2)cc1 ; c1ccc(-c2ccc(N(c3ccc4ccc(-c5cc6ccccc6c6ccccc56)cc4c3)c3ccc4oc5cc6ccccc6cc5c4c3)cc2)cc1]

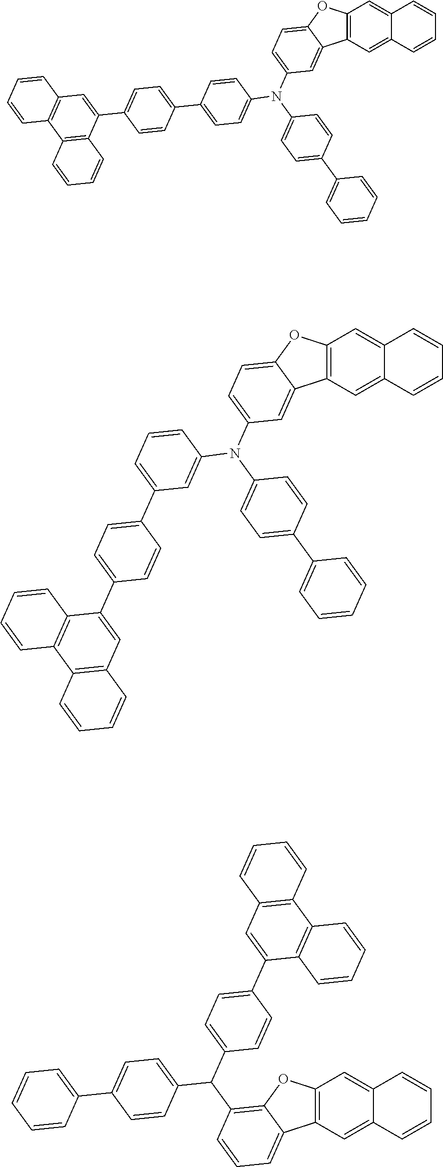 [SMILES: c1ccc(-c2ccc(N(c3ccc(-c4ccc(-c5cc6ccccc6c6ccccc56)cc4)cc3)c3ccc4oc5cc6ccccc6cc5c4c3)cc2)cc1 ; c1ccc(-c2ccc(N(c3cccc(-c4ccc(-c5cc6ccccc6c6ccccc56)cc4)c3)c3ccc4oc5cc6ccccc6cc5c4c3)cc2)cc1 ; c1ccc(-c2ccc(C(c3ccc(-c4cc5ccccc5c5ccccc45)cc3)c3cccc4c3oc3cc5ccccc5cc34)cc2)cc1]

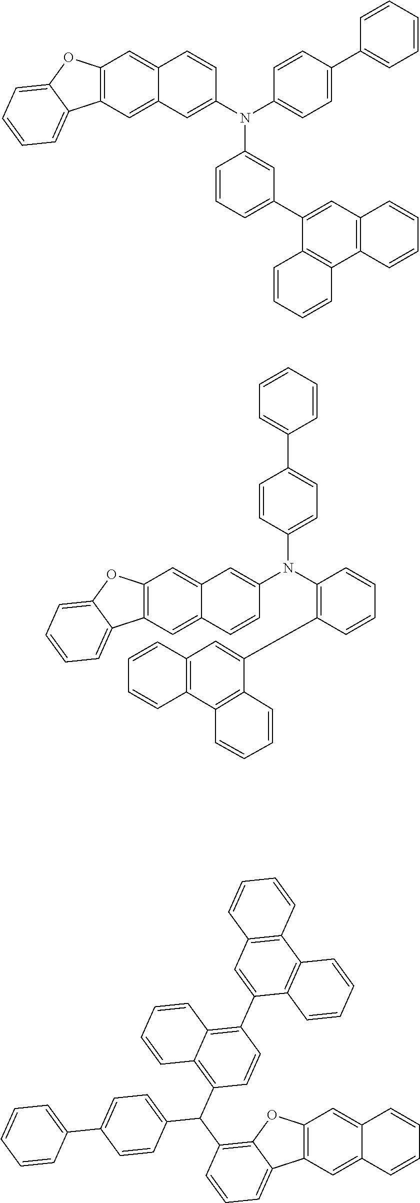 [SMILES: c1ccc(-c2ccc(N(c3cccc(-c4cc5ccccc5c5ccccc45)c3)c3ccc4cc5oc6ccccc6c5cc4c3)cc2)cc1 ; c1ccc(-c2ccc(N(c3ccc4cc5c(cc4c3)oc3ccccc35)c3ccccc3-c3cc4ccccc4c4ccccc34)cc2)cc1 ; c1ccc(-c2ccc(C(c3ccc(-c4cc5ccccc5c5ccccc45)c4ccccc34)c3cccc4c3oc3cc5ccccc5cc34)cc2)cc1]

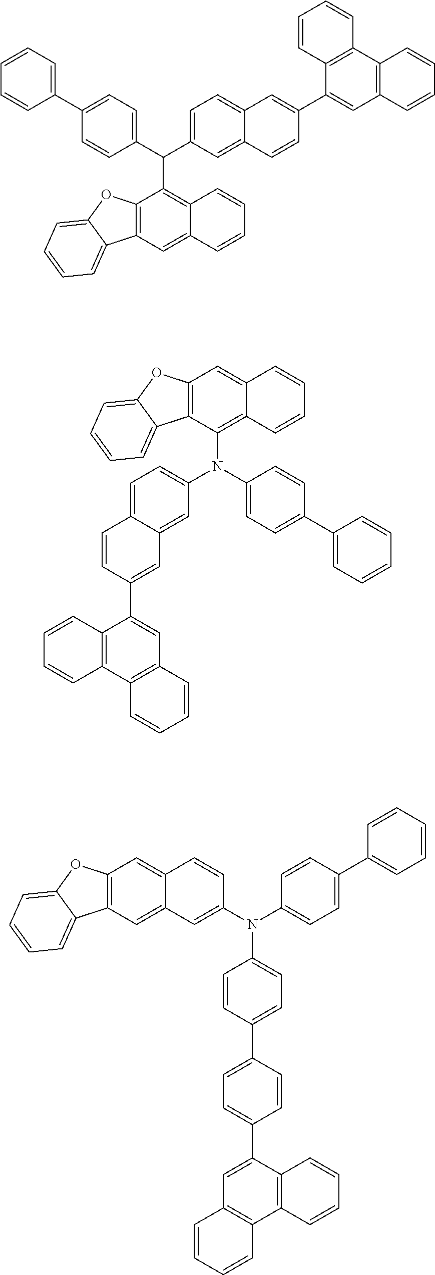 [SMILES: c1ccc(-c2ccc(C(c3ccc4cc(-c5cc6ccccc6c6ccccc56)ccc4c3)c3c4ccccc4cc4c3oc3ccccc34)cc2)cc1 ; c1ccc(-c2ccc(N(c3ccc4ccc(-c5cc6ccccc6c6ccccc56)cc4c3)c3c4ccccc4cc4oc5ccccc5c34)cc2)cc1 ; c1ccc(-c2ccc(N(c3ccc(-c4ccc(-c5cc6ccccc6c6ccccc56)cc4)cc3)c3ccc4cc5oc6ccccc6c5cc4c3)cc2)cc1]

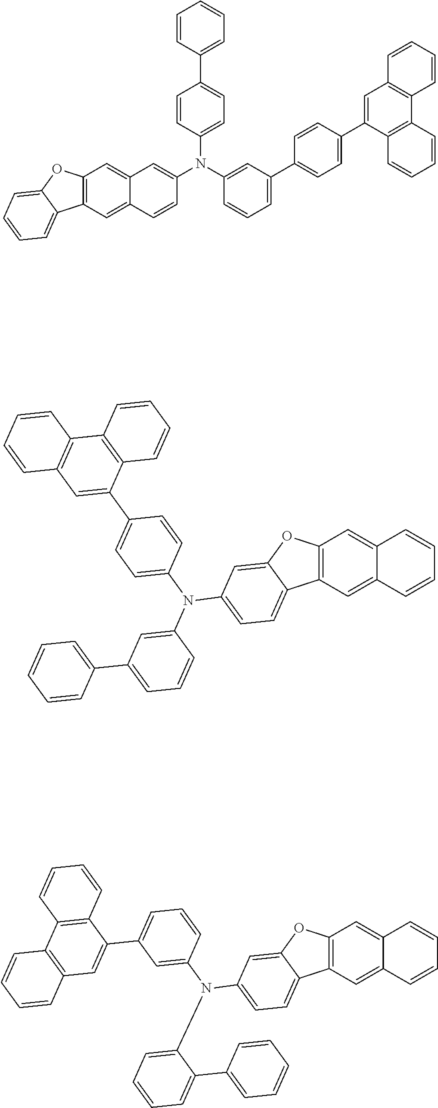 [SMILES: c1ccc(-c2ccc(N(c3cccc(-c4ccc(-c5cc6ccccc6c6ccccc56)cc4)c3)c3ccc4cc5c(cc4c3)oc3ccccc35)cc2)cc1 ; c1ccc(-c2cccc(N(c3ccc(-c4cc5ccccc5c5ccccc45)cc3)c3ccc4c(c3)oc3cc5ccccc5cc34)c2)cc1 ; c1ccc(-c2ccccc2N(c2cccc(-c3cc4ccccc4c4ccccc34)c2)c2ccc3c(c2)oc2cc4ccccc4cc23)cc1]

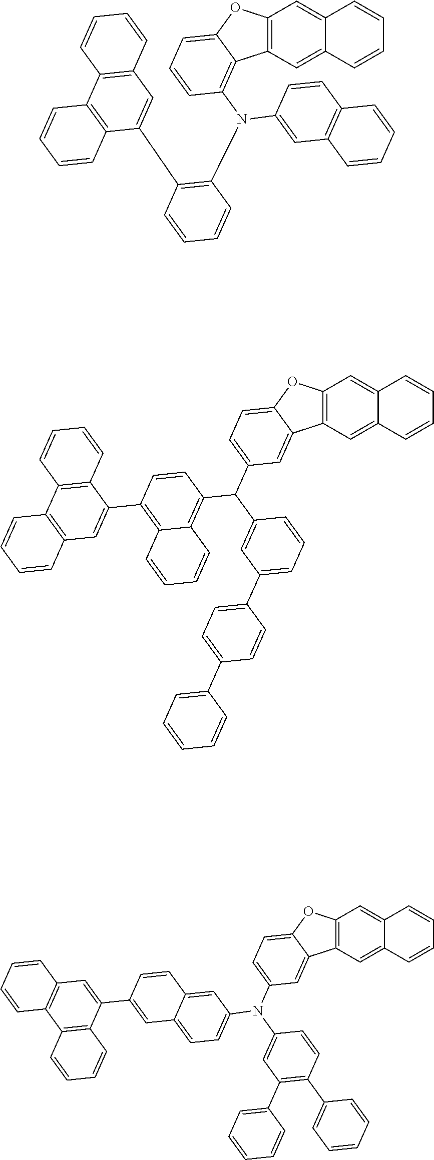 [SMILES: c1ccc(N(c2ccc3ccccc3c2)c2cccc3oc4cc5ccccc5cc4c23)c(-c2cc3ccccc3c3ccccc23)c1 ; c1ccc(-c2ccc(-c3cccc(C(c4ccc5oc6cc7ccccc7cc6c5c4)c4ccc(-c5cc6ccccc6c6ccccc56)c5ccccc45)c3)cc2)cc1 ; c1ccc(-c2ccc(N(c3ccc4cc(-c5cc6ccccc6c6ccccc56)ccc4c3)c3ccc4oc5cc6ccccc6cc5c4c3)cc2-c2ccccc2)cc1]

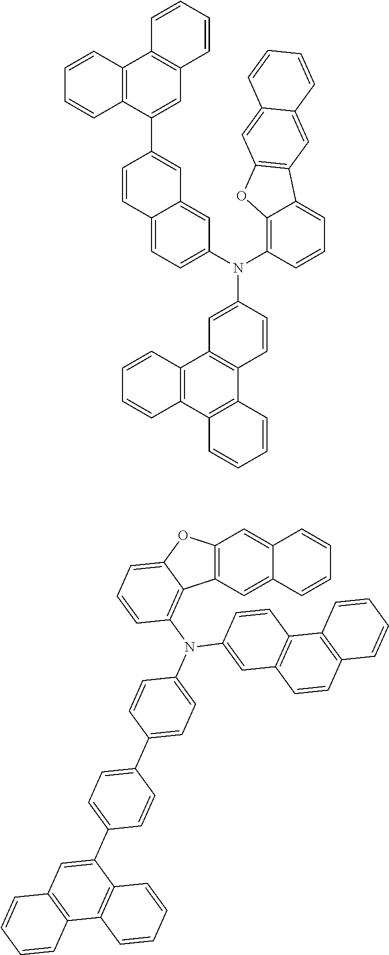 [SMILES: c1ccc2cc3c(cc2c1)oc1c(N(c2ccc4ccc(-c5cc6ccccc6c6ccccc56)cc4c2)c2ccc4c5ccccc5c5ccccc5c4c2)cccc13 ; c1ccc2cc3c(cc2c1)oc1cccc(N(c2ccc(-c4ccc(-c5cc6ccccc6c6ccccc56)cc4)cc2)c2ccc4c(ccc5ccccc54)c2)c13]

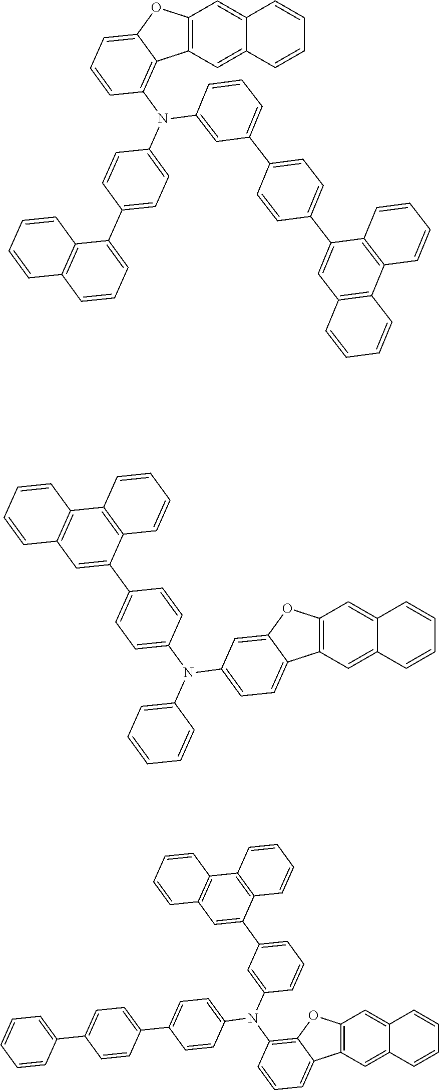 [SMILES: c1cc(-c2ccc(-c3cc4ccccc4c4ccccc34)cc2)cc(N(c2ccc(-c3cccc4ccccc34)cc2)c2cccc3oc4cc5ccccc5cc4c23)c1 ; c1ccc(N(c2ccc(-c3cc4ccccc4c4ccccc34)cc2)c2ccc3c(c2)oc2cc4ccccc4cc23)cc1 ; c1ccc(-c2ccc(-c3ccc(N(c4cccc(-c5cc6ccccc6c6ccccc56)c4)c4cccc5c4oc4cc6ccccc6cc45)cc3)cc2)cc1]

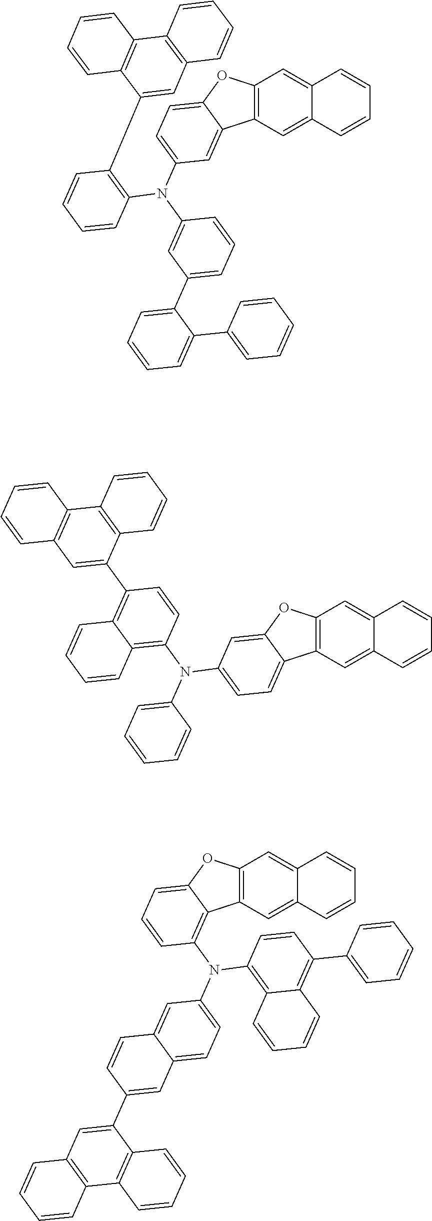 [SMILES: c1ccc(-c2ccccc2-c2cccc(N(c3ccc4oc5cc6ccccc6cc5c4c3)c3ccccc3-c3cc4ccccc4c4ccccc34)c2)cc1 ; c1ccc(N(c2ccc3c(c2)oc2cc4ccccc4cc23)c2ccc(-c3cc4ccccc4c4ccccc34)c3ccccc23)cc1 ; c1ccc(-c2ccc(N(c3ccc4cc(-c5cc6ccccc6c6ccccc56)ccc4c3)c3cccc4oc5cc6ccccc6cc5c34)c3ccccc23)cc1]

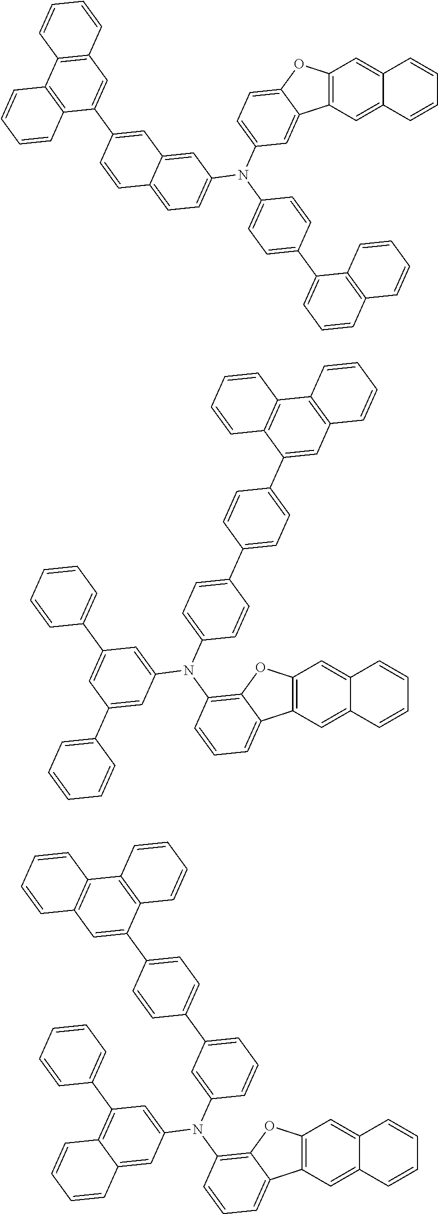

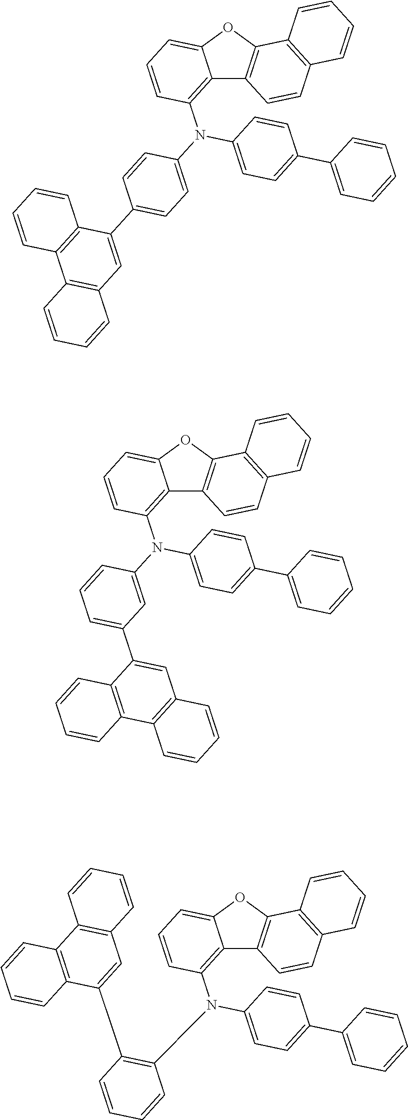 [SMILES: c1ccc(-c2ccc(N(c3ccc(-c4cc5ccccc5c5ccccc45)cc3)c3cccc4oc5c6ccccc6ccc5c34)cc2)cc1 ; c1ccc(-c2ccc(N(c3cccc(-c4cc5ccccc5c5ccccc45)c3)c3cccc4oc5c6ccccc6ccc5c34)cc2)cc1 ; c1ccc(-c2ccc(N(c3ccccc3-c3cc4ccccc4c4ccccc34)c3cccc4oc5c6ccccc6ccc5c34)cc2)cc1]

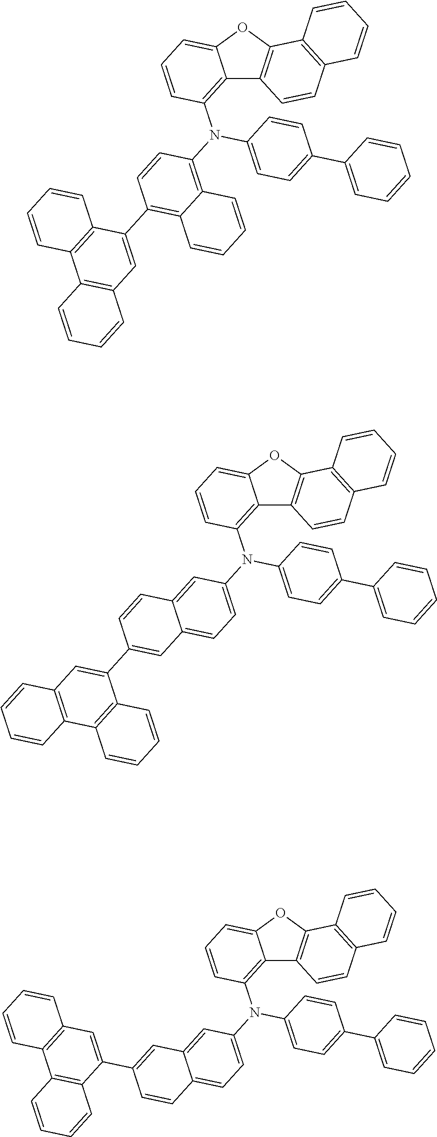 [SMILES: c1ccc(-c2ccc(N(c3ccc(-c4cc5ccccc5c5ccccc45)c4ccccc34)c3cccc4oc5c6ccccc6ccc5c34)cc2)cc1 ; c1ccc(-c2ccc(N(c3ccc4cc(-c5cc6ccccc6c6ccccc56)ccc4c3)c3cccc4oc5c6ccccc6ccc5c34)cc2)cc1 ; c1ccc(-c2ccc(N(c3ccc4ccc(-c5cc6ccccc6c6ccccc56)cc4c3)c3cccc4oc5c6ccccc6ccc5c34)cc2)cc1]

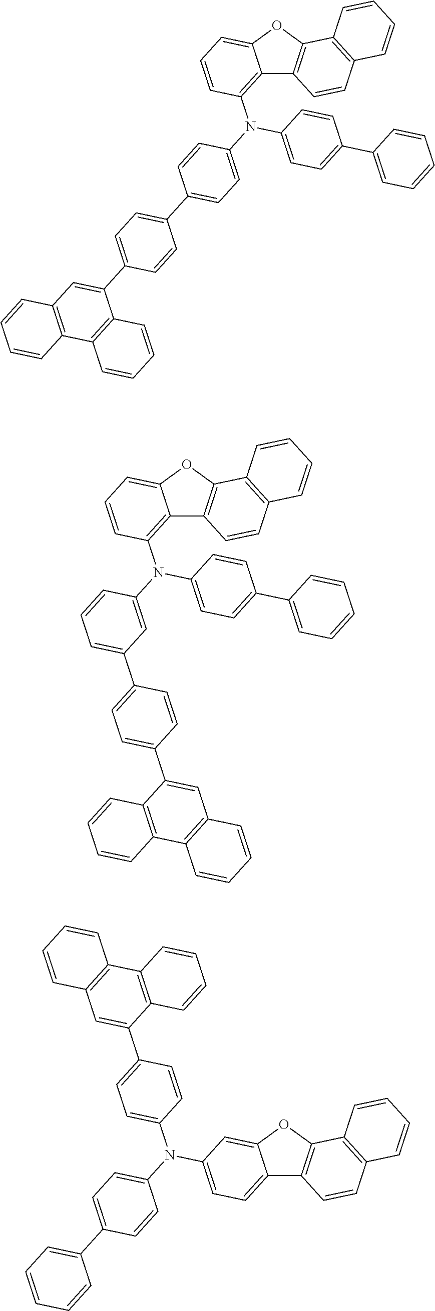 [SMILES: c1ccc(-c2ccc(N(c3ccc(-c4ccc(-c5cc6ccccc6c6ccccc56)cc4)cc3)c3cccc4oc5c6ccccc6ccc5c34)cc2)cc1 ; c1ccc(-c2ccc(N(c3cccc(-c4ccc(-c5cc6ccccc6c6ccccc56)cc4)c3)c3cccc4oc5c6ccccc6ccc5c34)cc2)cc1 ; c1ccc(-c2ccc(N(c3ccc(-c4cc5ccccc5c5ccccc45)cc3)c3ccc4c(c3)oc3c5ccccc5ccc43)cc2)cc1]

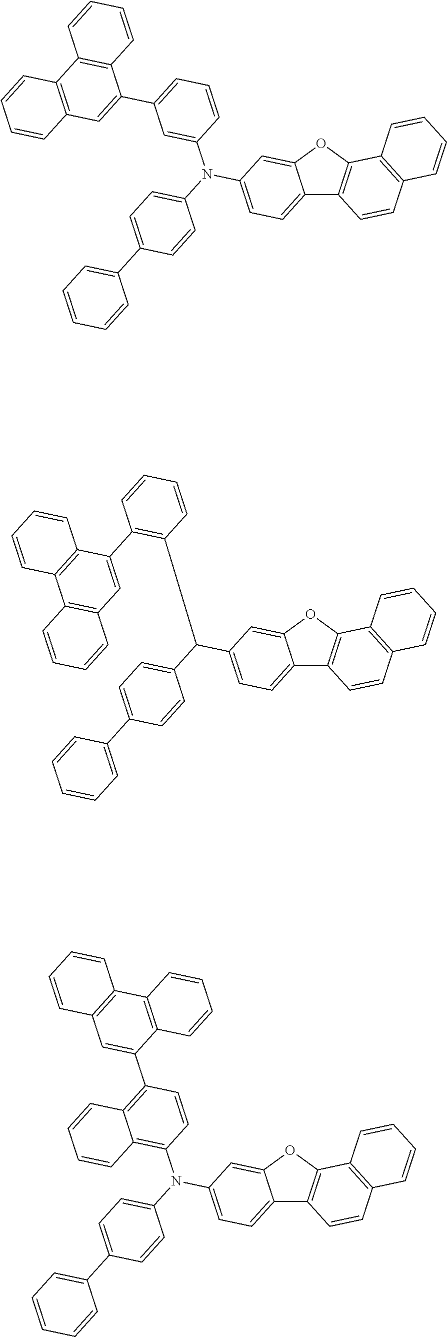 [SMILES: c1ccc(-c2ccc(N(c3cccc(-c4cc5ccccc5c5ccccc45)c3)c3ccc4c(c3)oc3c5ccccc5ccc43)cc2)cc1 ; c1ccc(-c2ccc(C(c3ccc4c(c3)oc3c5ccccc5ccc43)c3ccccc3-c3cc4ccccc4c4ccccc34)cc2)cc1 ; c1ccc(-c2ccc(N(c3ccc4c(c3)oc3c5ccccc5ccc43)c3ccc(-c4cc5ccccc5c5ccccc45)c4ccccc34)cc2)cc1]

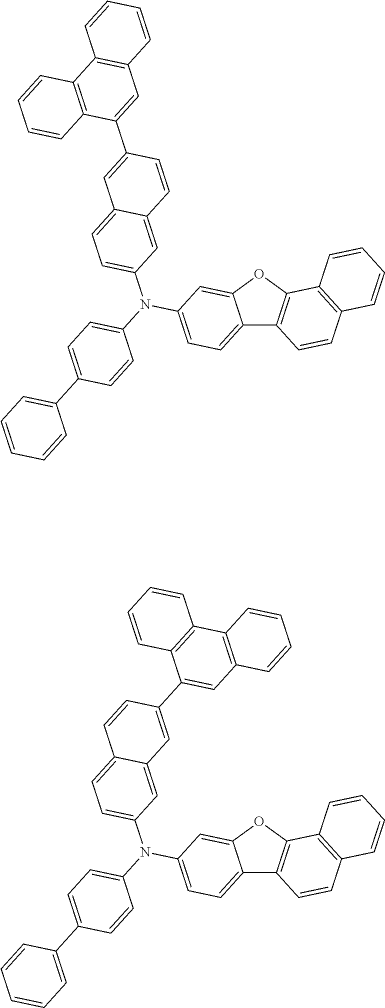 [SMILES: c1ccc(-c2ccc(N(c3ccc4cc(-c5cc6ccccc6c6ccccc56)ccc4c3)c3ccc4c(c3)oc3c5ccccc5ccc43)cc2)cc1 ; c1ccc(-c2ccc(N(c3ccc4ccc(-c5cc6ccccc6c6ccccc56)cc4c3)c3ccc4c(c3)oc3c5ccccc5ccc43)cc2)cc1]

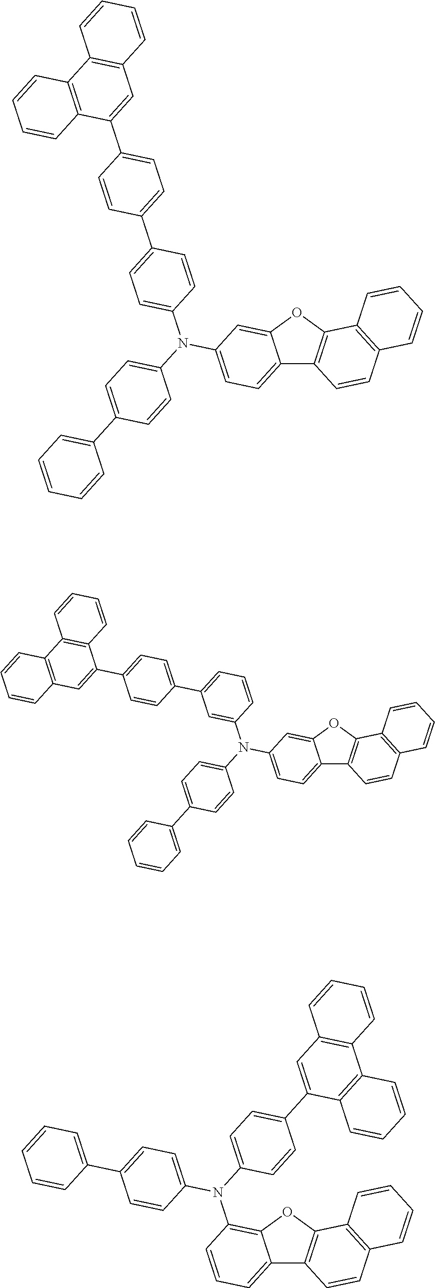 [SMILES: c1ccc(-c2ccc(N(c3ccc(-c4ccc(-c5cc6ccccc6c6ccccc56)cc4)cc3)c3ccc4c(c3)oc3c5ccccc5ccc43)cc2)cc1 ; c1ccc(-c2ccc(N(c3cccc(-c4ccc(-c5cc6ccccc6c6ccccc56)cc4)c3)c3ccc4c(c3)oc3c5ccccc5ccc43)cc2)cc1 ; c1ccc(-c2ccc(N(c3ccc(-c4cc5ccccc5c5ccccc45)cc3)c3cccc4c3oc3c5ccccc5ccc43)cc2)cc1]

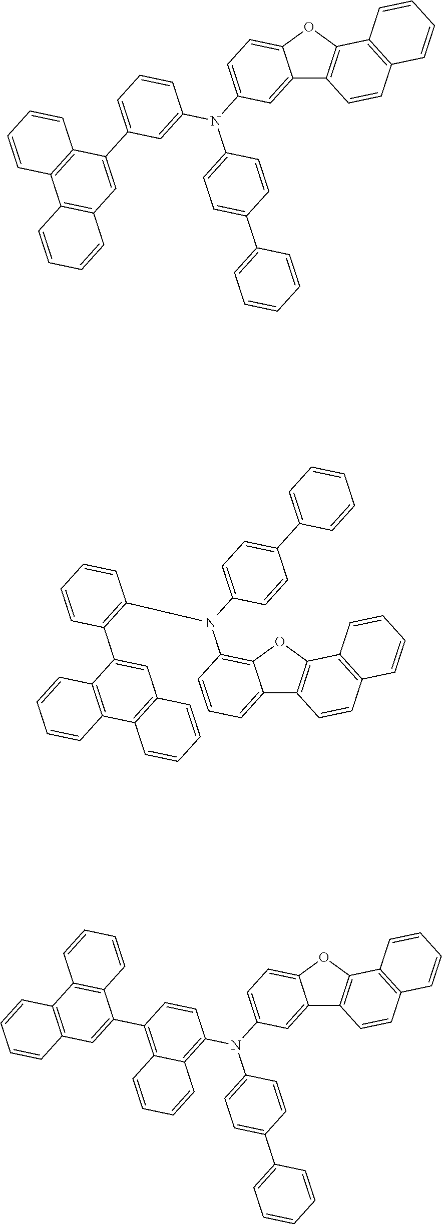 [SMILES: c1ccc(-c2ccc(N(c3cccc(-c4cc5ccccc5c5ccccc45)c3)c3ccc4oc5c6ccccc6ccc5c4c3)cc2)cc1 ; c1ccc(-c2ccc(N(c3ccccc3-c3cc4ccccc4c4ccccc34)c3cccc4c3oc3c5ccccc5ccc43)cc2)cc1 ; c1ccc(-c2ccc(N(c3ccc4oc5c6ccccc6ccc5c4c3)c3ccc(-c4cc5ccccc5c5ccccc45)c4ccccc34)cc2)cc1]

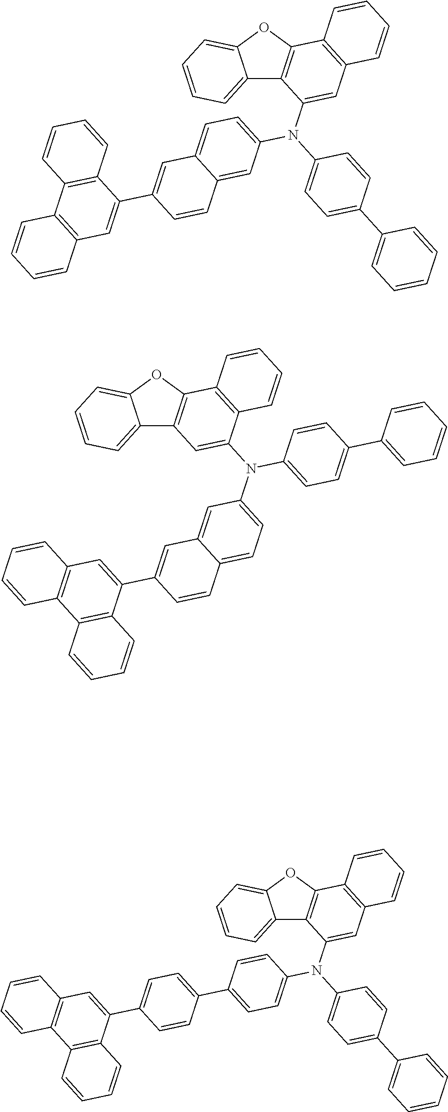 [SMILES: c1ccc(-c2ccc(N(c3ccc4cc(-c5cc6ccccc6c6ccccc56)ccc4c3)c3cc4ccccc4c4oc5ccccc5c34)cc2)cc1 ; c1ccc(-c2ccc(N(c3ccc4ccc(-c5cc6ccccc6c6ccccc56)cc4c3)c3cc4c5ccccc5oc4c4ccccc34)cc2)cc1 ; c1ccc(-c2ccc(N(c3ccc(-c4ccc(-c5cc6ccccc6c6ccccc56)cc4)cc3)c3cc4ccccc4c4oc5ccccc5c34)cc2)cc1]

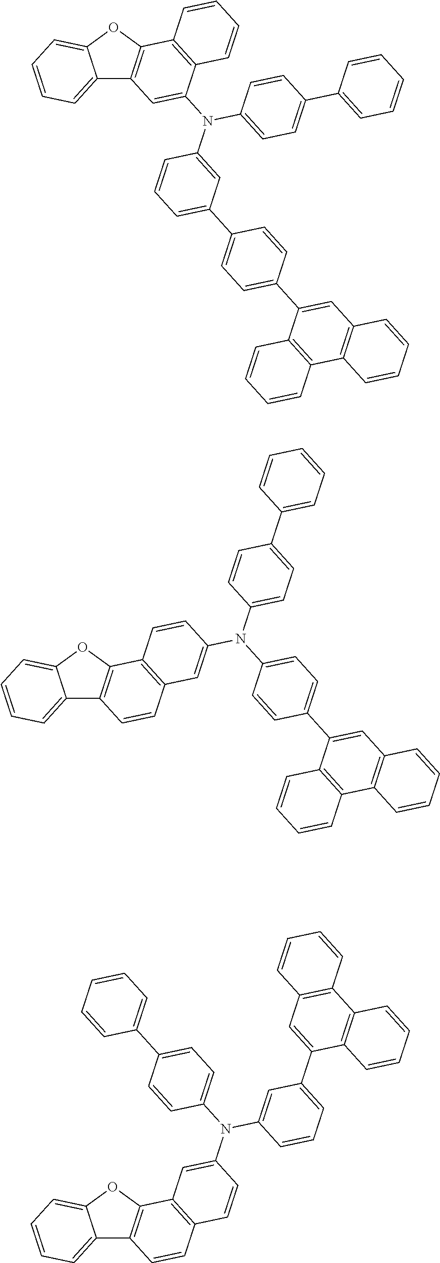 [SMILES: c1ccc(-c2ccc(N(c3cccc(-c4ccc(-c5cc6ccccc6c6ccccc56)cc4)c3)c3cc4c5ccccc5oc4c4ccccc34)cc2)cc1 ; c1ccc(-c2ccc(N(c3ccc(-c4cc5ccccc5c5ccccc45)cc3)c3ccc4c(ccc5c6ccccc6oc45)c3)cc2)cc1 ; c1ccc(-c2ccc(N(c3cccc(-c4cc5ccccc5c5ccccc45)c3)c3ccc4ccc5c6ccccc6oc5c4c3)cc2)cc1]

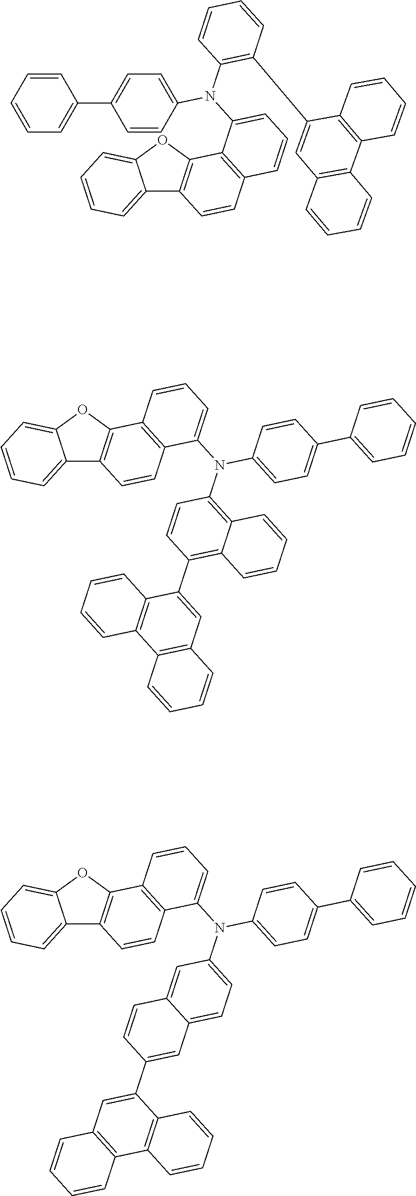 [SMILES: c1ccc(-c2ccc(N(c3ccccc3-c3cc4ccccc4c4ccccc34)c3cccc4ccc5c6ccccc6oc5c34)cc2)cc1 ; c1ccc(-c2ccc(N(c3ccc(-c4cc5ccccc5c5ccccc45)c4ccccc34)c3cccc4c3ccc3c5ccccc5oc43)cc2)cc1 ; c1ccc(-c2ccc(N(c3ccc4cc(-c5cc6ccccc6c6ccccc56)ccc4c3)c3cccc4c3ccc3c5ccccc5oc43)cc2)cc1]

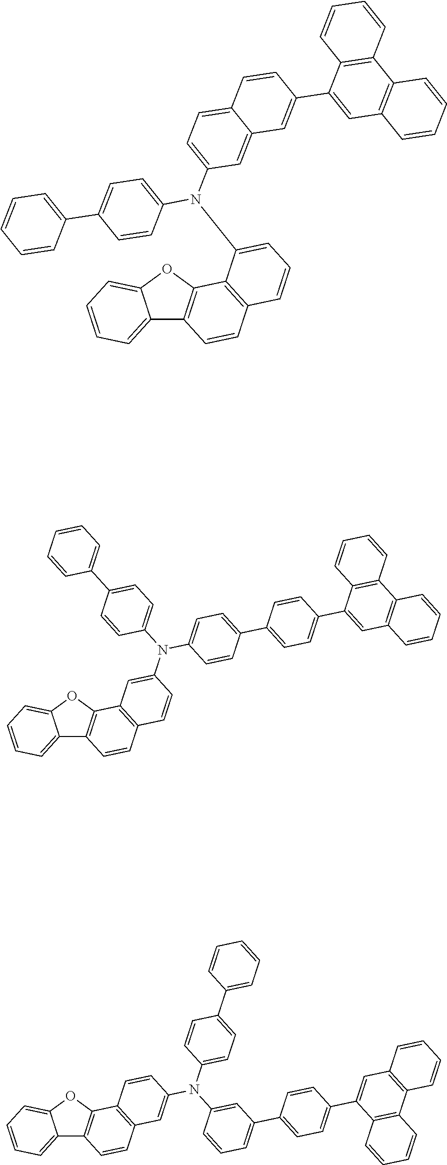 [SMILES: c1ccc(-c2ccc(N(c3ccc4ccc(-c5cc6ccccc6c6ccccc56)cc4c3)c3cccc4ccc5c6ccccc6oc5c34)cc2)cc1 ; c1ccc(-c2ccc(N(c3ccc(-c4ccc(-c5cc6ccccc6c6ccccc56)cc4)cc3)c3ccc4ccc5c6ccccc6oc5c4c3)cc2)cc1 ; c1ccc(-c2ccc(N(c3cccc(-c4ccc(-c5cc6ccccc6c6ccccc56)cc4)c3)c3ccc4c(ccc5c6ccccc6oc45)c3)cc2)cc1]

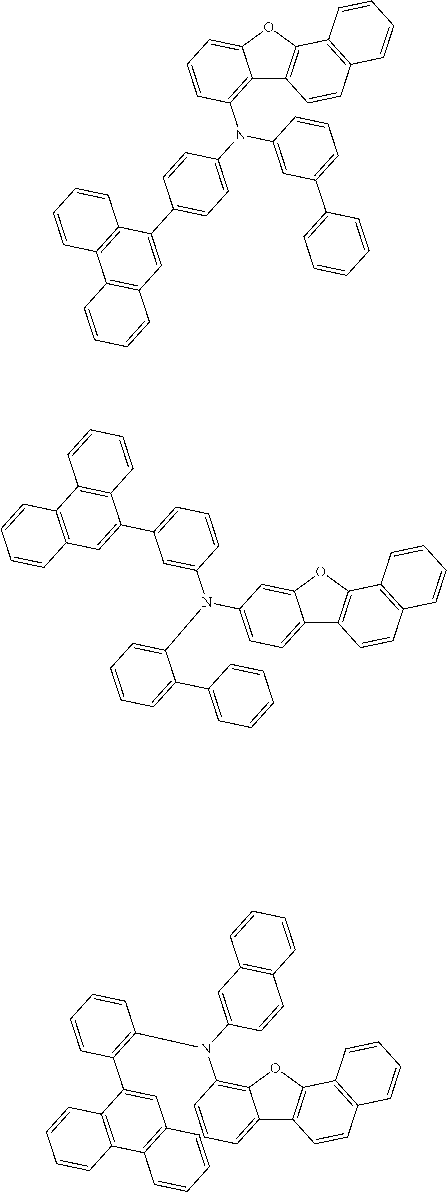 [SMILES: c1ccc(-c2cccc(N(c3ccc(-c4cc5ccccc5c5ccccc45)cc3)c3cccc4oc5c6ccccc6ccc5c34)c2)cc1 ; c1ccc(-c2ccccc2N(c2cccc(-c3cc4ccccc4c4ccccc34)c2)c2ccc3c(c2)oc2c4ccccc4ccc32)cc1 ; c1ccc(N(c2ccc3ccccc3c2)c2cccc3c2oc2c4ccccc4ccc32)c(-c2cc3ccccc3c3ccccc23)c1]

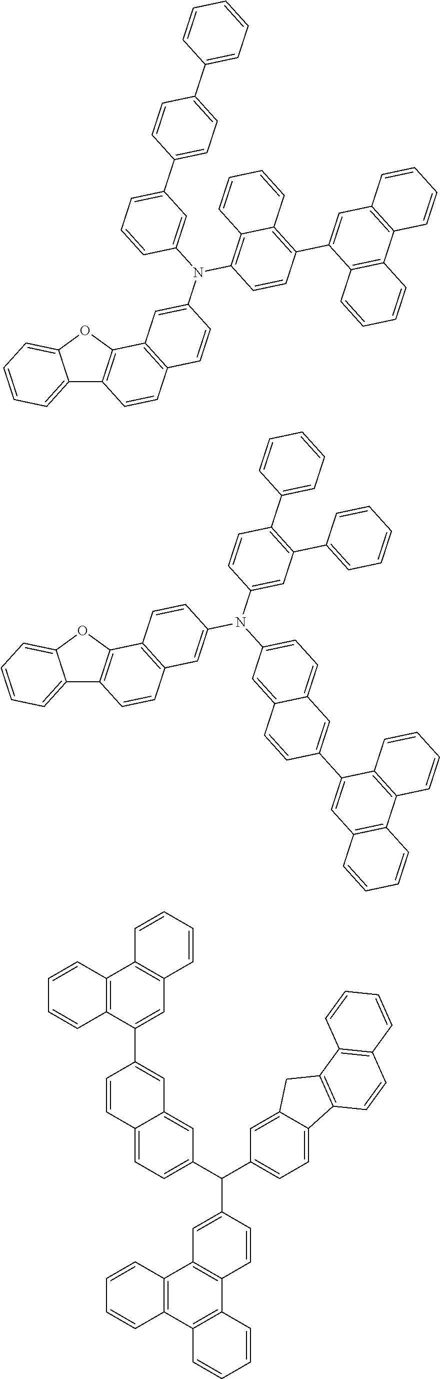

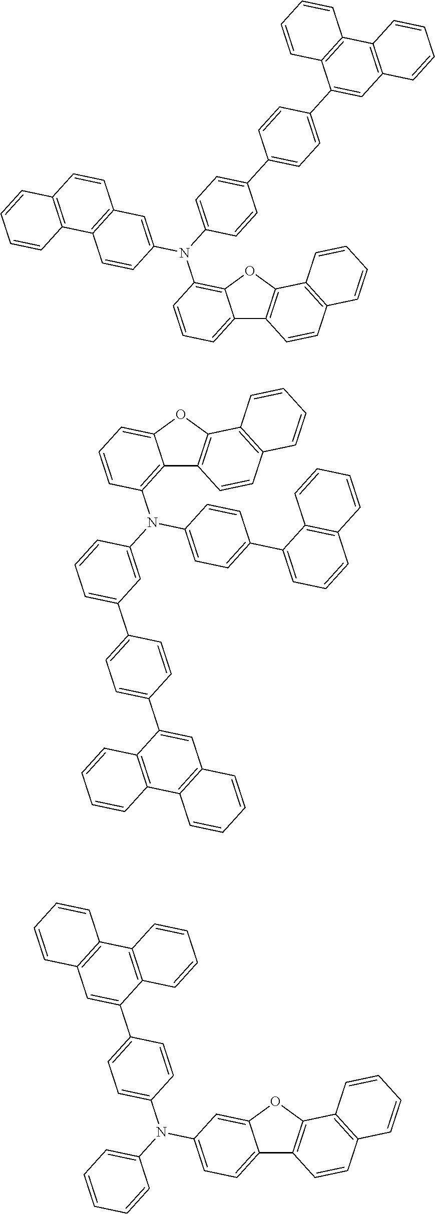 [SMILES: c1ccc2c(c1)ccc1cc(N(c3ccc(-c4ccc(-c5cc6ccccc6c6ccccc56)cc4)cc3)c3cccc4c3oc3c5ccccc5ccc43)ccc12 ; c1cc(-c2ccc(-c3cc4ccccc4c4ccccc34)cc2)cc(N(c2ccc(-c3cccc4ccccc34)cc2)c2cccc3oc4c5ccccc5ccc4c23)c1 ; c1ccc(N(c2ccc(-c3cc4ccccc4c4ccccc34)cc2)c2ccc3c(c2)oc2c4ccccc4ccc32)cc1]

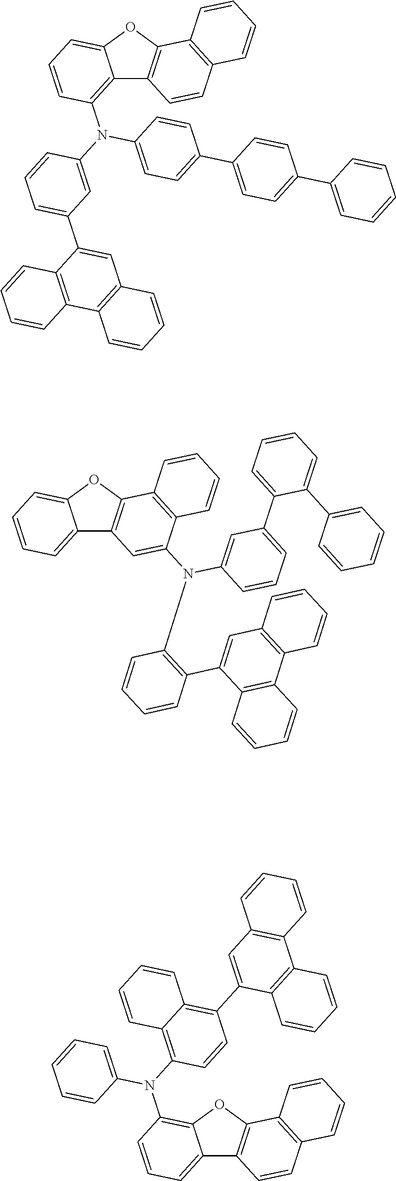 [SMILES: c1ccc(-c2ccc(-c3ccc(N(c4cccc(-c5cc6ccccc6c6ccccc56)c4)c4cccc5oc6c7ccccc7ccc6c45)cc3)cc2)cc1 ; c1ccc(-c2ccccc2-c2cccc(N(c3ccccc3-c3cc4ccccc4c4ccccc34)c3cc4c5ccccc5oc4c4ccccc34)c2)cc1 ; c1ccc(N(c2ccc(-c3cc4ccccc4c4ccccc34)c3ccccc23)c2cccc3c2oc2c4ccccc4ccc32)cc1]

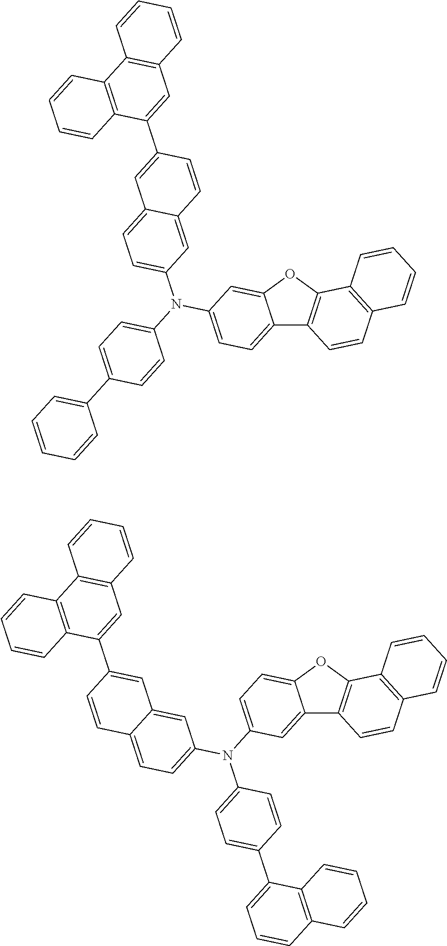 [SMILES: c1ccc(-c2ccc(N(c3ccc4cc(-c5cc6ccccc6c6ccccc56)ccc4c3)c3ccc4c(c3)oc3c5ccccc5ccc43)cc2)cc1 ; c1ccc2c(-c3ccc(N(c4ccc5ccc(-c6cc7ccccc7c7ccccc67)cc5c4)c4ccc5oc6c7ccccc7ccc6c5c4)cc3)cccc2c1]

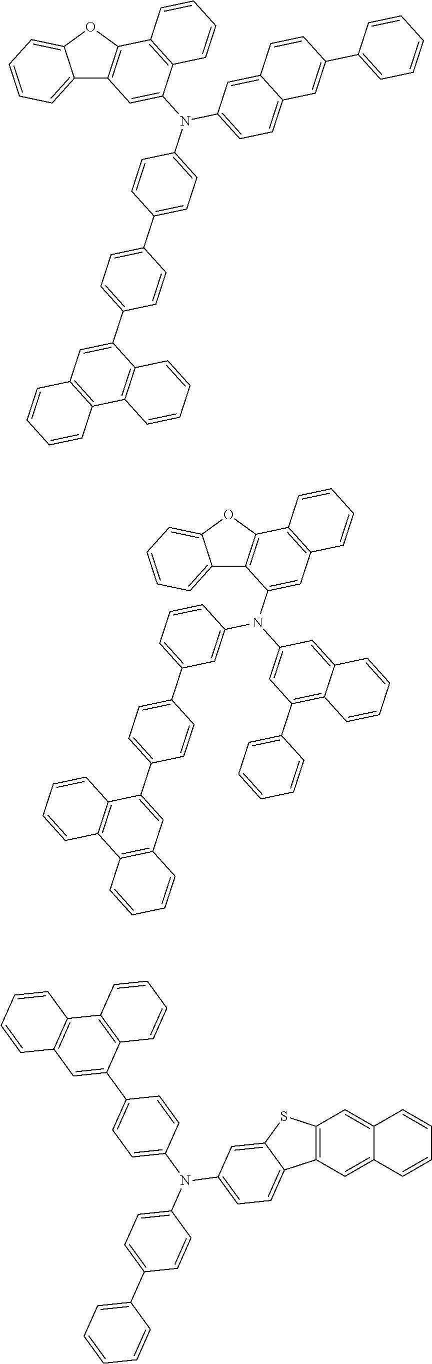 [SMILES: c1ccc(-c2ccc3cc(N(c4ccc(-c5ccc(-c6cc7ccccc7c7ccccc67)cc5)cc4)c4cc5c6ccccc6oc5c5ccccc45)ccc3c2)cc1 ; c1ccc(-c2cc(N(c3cccc(-c4ccc(-c5cc6ccccc6c6ccccc56)cc4)c3)c3cc4ccccc4c4oc5ccccc5c34)cc3ccccc23)cc1 ; c1ccc(-c2ccc(N(c3ccc(-c4cc5ccccc5c5ccccc45)cc3)c3ccc4c(c3)sc3cc5ccccc5cc34)cc2)cc1]

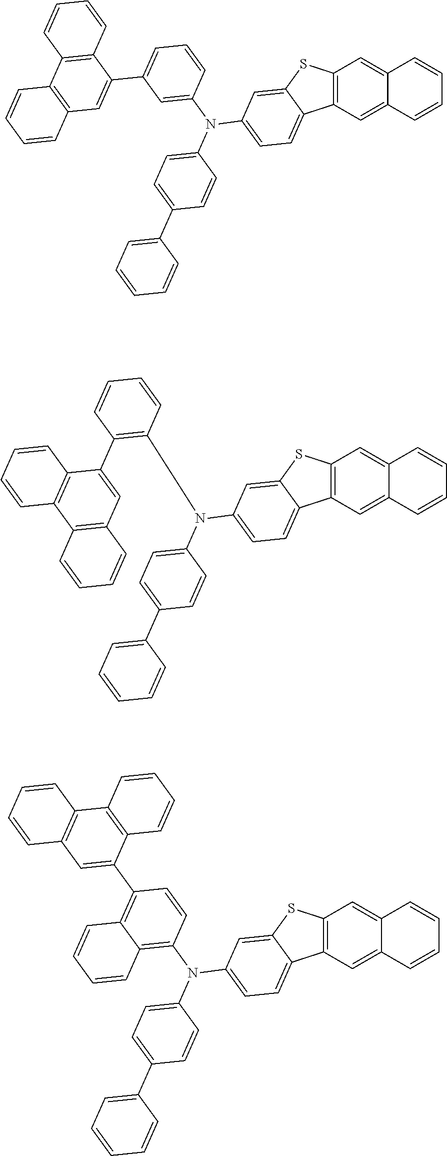 [SMILES: c1ccc(-c2ccc(N(c3cccc(-c4cc5ccccc5c5ccccc45)c3)c3ccc4c(c3)sc3cc5ccccc5cc34)cc2)cc1 ; c1ccc(-c2ccc(N(c3ccc4c(c3)sc3cc5ccccc5cc34)c3ccccc3-c3cc4ccccc4c4ccccc34)cc2)cc1 ; c1ccc(-c2ccc(N(c3ccc4c(c3)sc3cc5ccccc5cc34)c3ccc(-c4cc5ccccc5c5ccccc45)c4ccccc34)cc2)cc1]

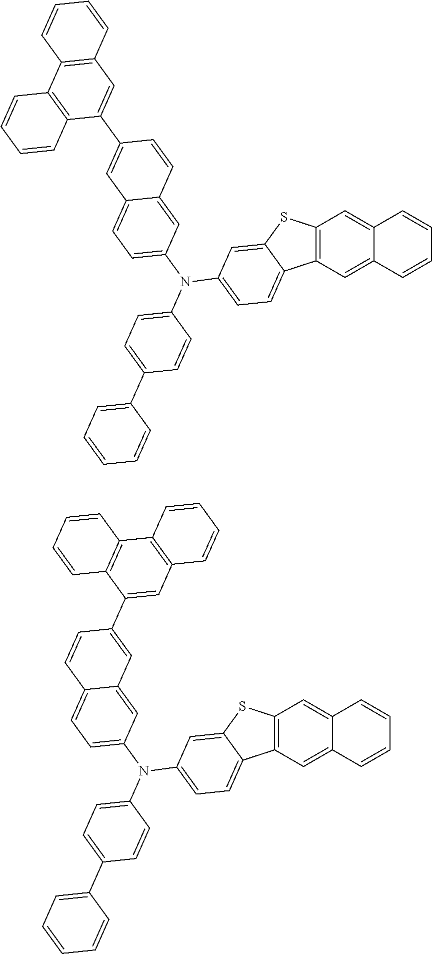 [SMILES: c1ccc(-c2ccc(N(c3ccc4cc(-c5cc6ccccc6c6ccccc56)ccc4c3)c3ccc4c(c3)sc3cc5ccccc5cc34)cc2)cc1 ; c1ccc(-c2ccc(N(c3ccc4ccc(-c5cc6ccccc6c6ccccc56)cc4c3)c3ccc4c(c3)sc3cc5ccccc5cc34)cc2)cc1]

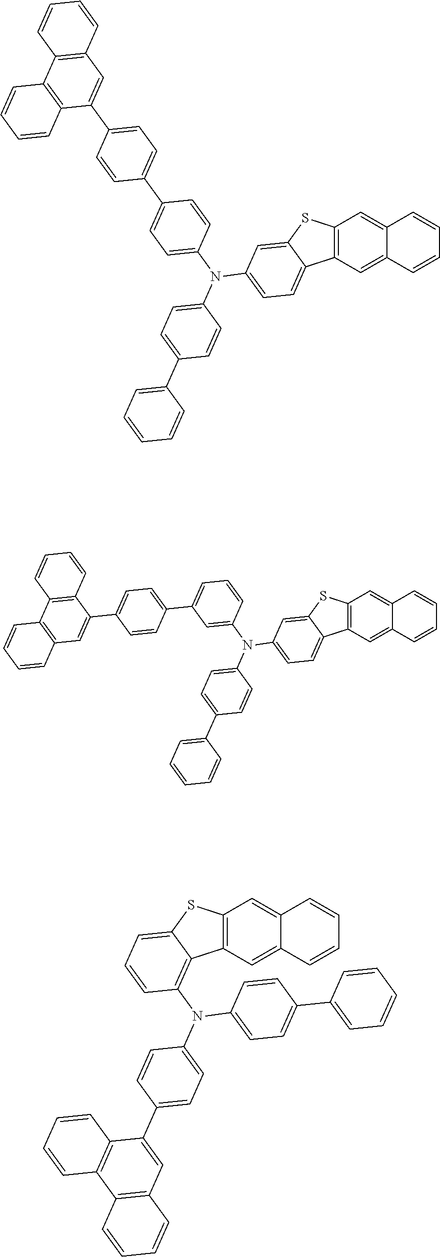 [SMILES: c1ccc(-c2ccc(N(c3ccc(-c4ccc(-c5cc6ccccc6c6ccccc56)cc4)cc3)c3ccc4c(c3)sc3cc5ccccc5cc34)cc2)cc1 ; c1ccc(-c2ccc(N(c3cccc(-c4ccc(-c5cc6ccccc6c6ccccc56)cc4)c3)c3ccc4c(c3)sc3cc5ccccc5cc34)cc2)cc1 ; c1ccc(-c2ccc(N(c3ccc(-c4cc5ccccc5c5ccccc45)cc3)c3cccc4sc5cc6ccccc6cc5c34)cc2)cc1]

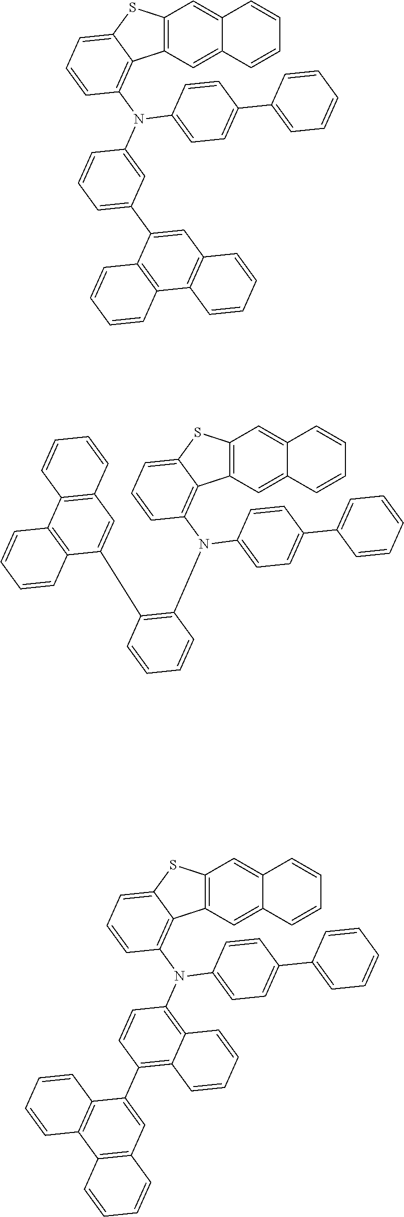 [SMILES: c1ccc(-c2ccc(N(c3cccc(-c4cc5ccccc5c5ccccc45)c3)c3cccc4sc5cc6ccccc6cc5c34)cc2)cc1 ; c1ccc(-c2ccc(N(c3ccccc3-c3cc4ccccc4c4ccccc34)c3cccc4sc5cc6ccccc6cc5c34)cc2)cc1 ; c1ccc(-c2ccc(N(c3ccc(-c4cc5ccccc5c5ccccc45)c4ccccc34)c3cccc4sc5cc6ccccc6cc5c34)cc2)cc1]

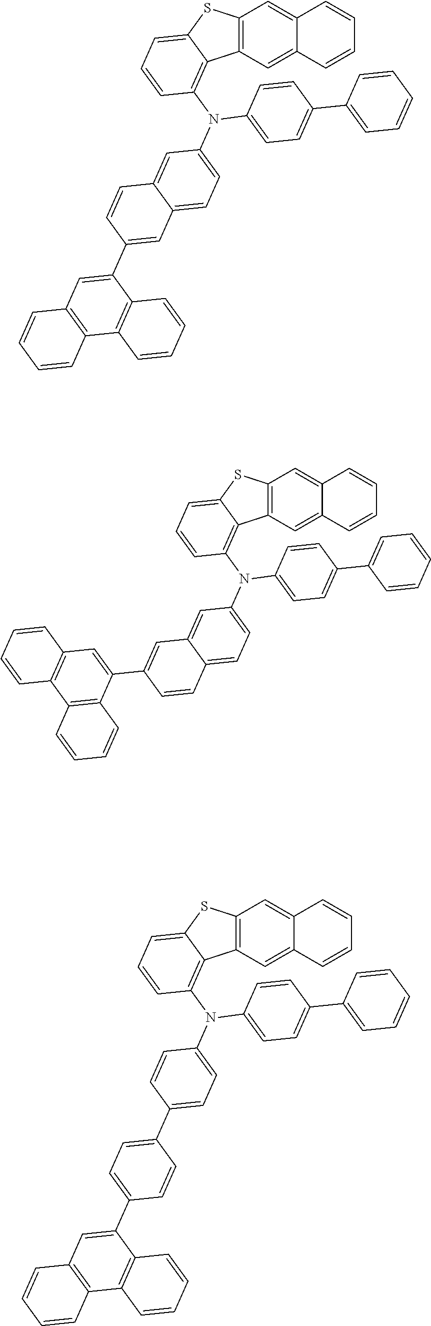 [SMILES: c1ccc(-c2ccc(N(c3ccc4cc(-c5cc6ccccc6c6ccccc56)ccc4c3)c3cccc4sc5cc6ccccc6cc5c34)cc2)cc1 ; c1ccc(-c2ccc(N(c3ccc4ccc(-c5cc6ccccc6c6ccccc56)cc4c3)c3cccc4sc5cc6ccccc6cc5c34)cc2)cc1 ; c1ccc(-c2ccc(N(c3ccc(-c4ccc(-c5cc6ccccc6c6ccccc56)cc4)cc3)c3cccc4sc5cc6ccccc6cc5c34)cc2)cc1]

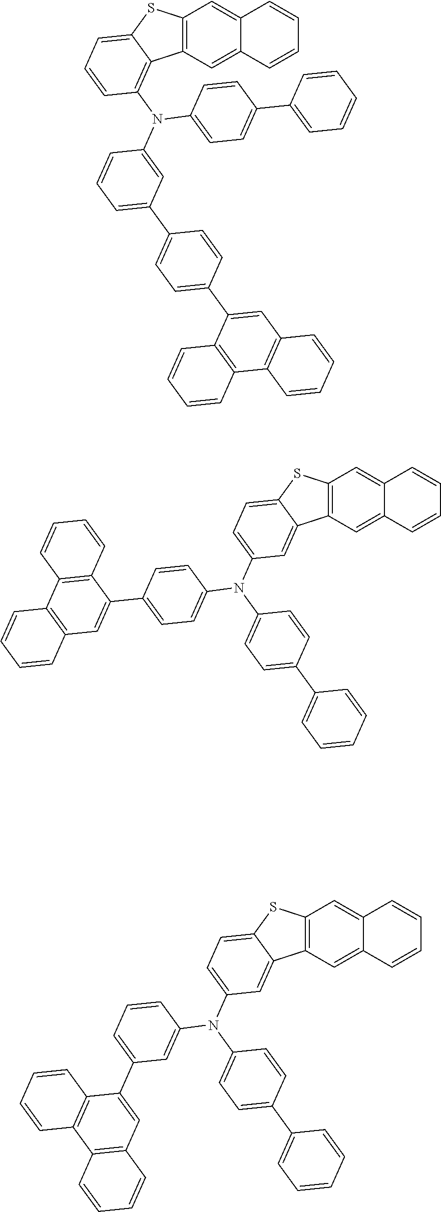 [SMILES: c1ccc(-c2ccc(N(c3cccc(-c4ccc(-c5cc6ccccc6c6ccccc56)cc4)c3)c3cccc4sc5cc6ccccc6cc5c34)cc2)cc1 ; c1ccc(-c2ccc(N(c3ccc(-c4cc5ccccc5c5ccccc45)cc3)c3ccc4sc5cc6ccccc6cc5c4c3)cc2)cc1 ; c1ccc(-c2ccc(N(c3cccc(-c4cc5ccccc5c5ccccc45)c3)c3ccc4sc5cc6ccccc6cc5c4c3)cc2)cc1]

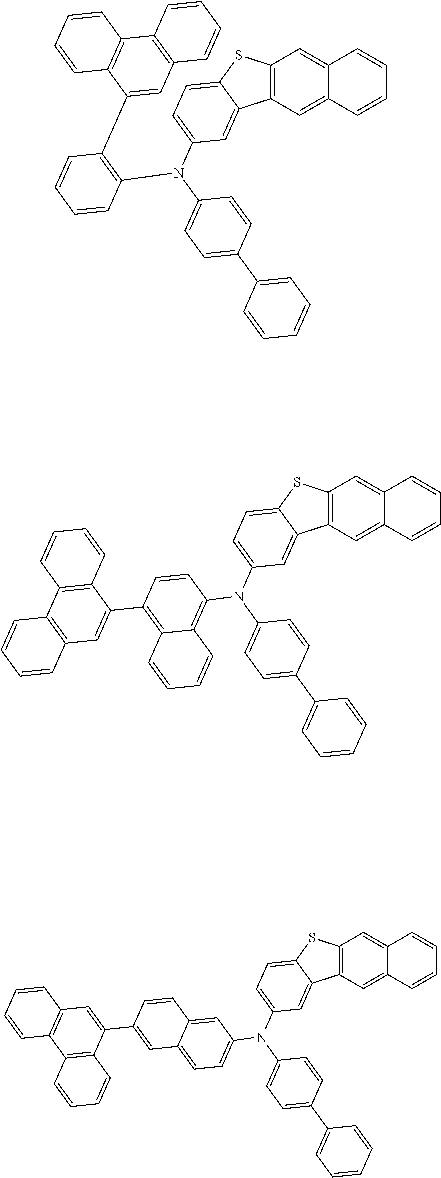 [SMILES: c1ccc(-c2ccc(N(c3ccc4sc5cc6ccccc6cc5c4c3)c3ccccc3-c3cc4ccccc4c4ccccc34)cc2)cc1 ; c1ccc(-c2ccc(N(c3ccc4sc5cc6ccccc6cc5c4c3)c3ccc(-c4cc5ccccc5c5ccccc45)c4ccccc34)cc2)cc1 ; c1ccc(-c2ccc(N(c3ccc4cc(-c5cc6ccccc6c6ccccc56)ccc4c3)c3ccc4sc5cc6ccccc6cc5c4c3)cc2)cc1]

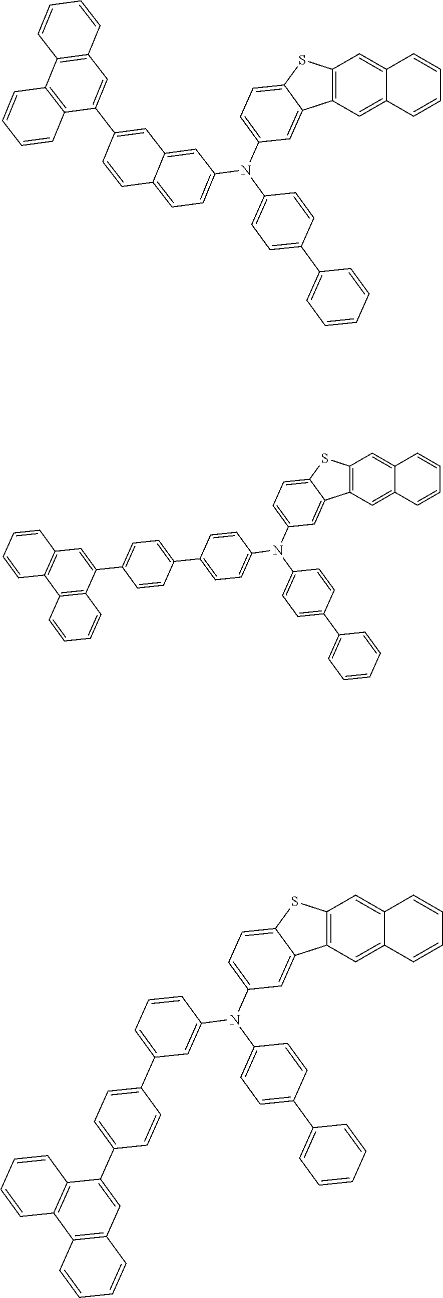 [SMILES: c1ccc(-c2ccc(N(c3ccc4ccc(-c5cc6ccccc6c6ccccc56)cc4c3)c3ccc4sc5cc6ccccc6cc5c4c3)cc2)cc1 ; c1ccc(-c2ccc(N(c3ccc(-c4ccc(-c5cc6ccccc6c6ccccc56)cc4)cc3)c3ccc4sc5cc6ccccc6cc5c4c3)cc2)cc1 ; c1ccc(-c2ccc(N(c3cccc(-c4ccc(-c5cc6ccccc6c6ccccc56)cc4)c3)c3ccc4sc5cc6ccccc6cc5c4c3)cc2)cc1]

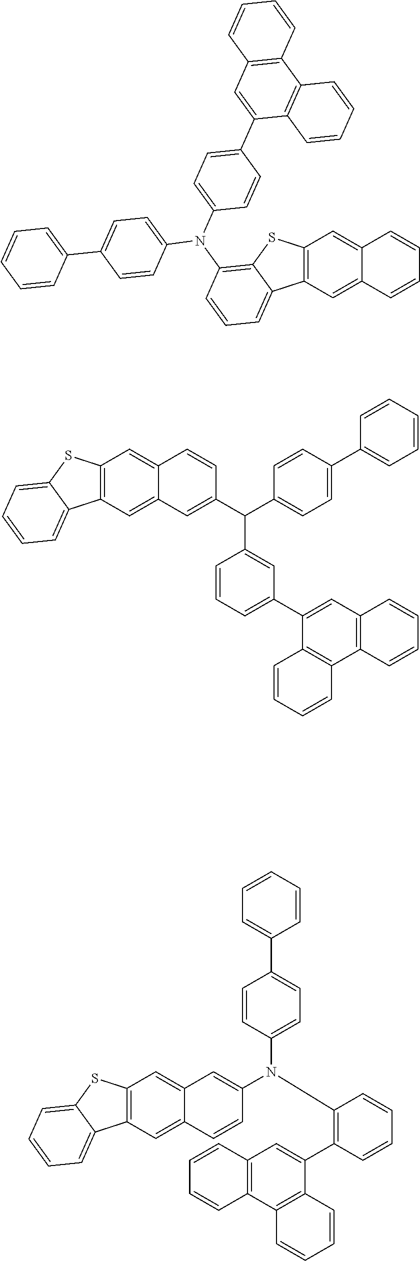 [SMILES: c1ccc(-c2ccc(N(c3ccc(-c4cc5ccccc5c5ccccc45)cc3)c3cccc4c3sc3cc5ccccc5cc34)cc2)cc1 ; c1ccc(-c2ccc(C(c3cccc(-c4cc5ccccc5c5ccccc45)c3)c3ccc4cc5sc6ccccc6c5cc4c3)cc2)cc1 ; c1ccc(-c2ccc(N(c3ccc4cc5c(cc4c3)sc3ccccc35)c3ccccc3-c3cc4ccccc4c4ccccc34)cc2)cc1]

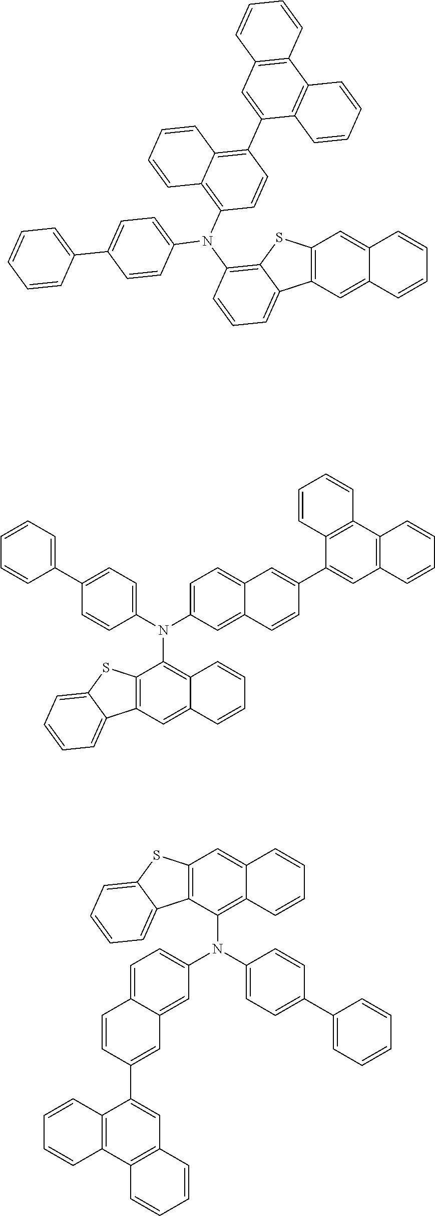 [SMILES: c1ccc(-c2ccc(N(c3ccc(-c4cc5ccccc5c5ccccc45)c4ccccc34)c3cccc4c3sc3cc5ccccc5cc34)cc2)cc1 ; c1ccc(-c2ccc(N(c3ccc4cc(-c5cc6ccccc6c6ccccc56)ccc4c3)c3c4ccccc4cc4c3sc3ccccc34)cc2)cc1 ; c1ccc(-c2ccc(N(c3ccc4ccc(-c5cc6ccccc6c6ccccc56)cc4c3)c3c4ccccc4cc4sc5ccccc5c34)cc2)cc1]

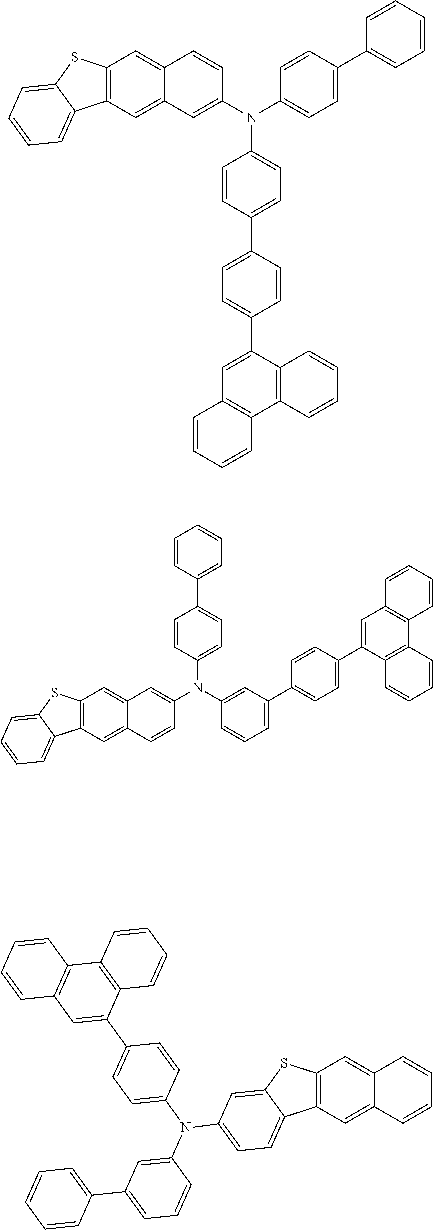 [SMILES: c1ccc(-c2ccc(N(c3ccc(-c4ccc(-c5cc6ccccc6c6ccccc56)cc4)cc3)c3ccc4cc5sc6ccccc6c5cc4c3)cc2)cc1 ; c1ccc(-c2ccc(N(c3cccc(-c4ccc(-c5cc6ccccc6c6ccccc56)cc4)c3)c3ccc4cc5c(cc4c3)sc3ccccc35)cc2)cc1 ; c1ccc(-c2cccc(N(c3ccc(-c4cc5ccccc5c5ccccc45)cc3)c3ccc4c(c3)sc3cc5ccccc5cc34)c2)cc1]

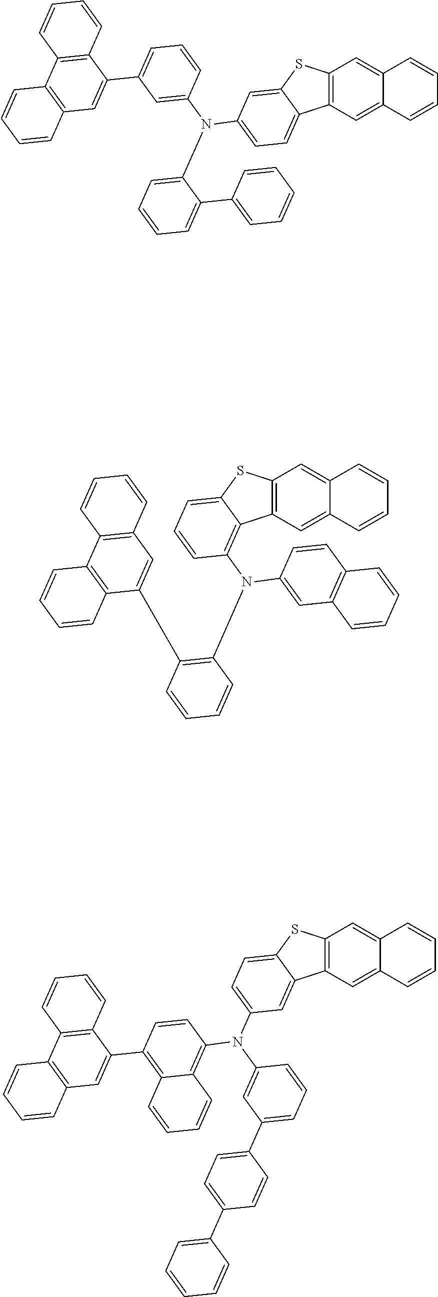 [SMILES: c1ccc(-c2ccccc2N(c2cccc(-c3cc4ccccc4c4ccccc34)c2)c2ccc3c(c2)sc2cc4ccccc4cc23)cc1 ; c1ccc(N(c2ccc3ccccc3c2)c2cccc3sc4cc5ccccc5cc4c23)c(-c2cc3ccccc3c3ccccc23)c1 ; c1ccc(-c2ccc(-c3cccc(N(c4ccc5sc6cc7ccccc7cc6c5c4)c4ccc(-c5cc6ccccc6c6ccccc56)c5ccccc45)c3)cc2)cc1]

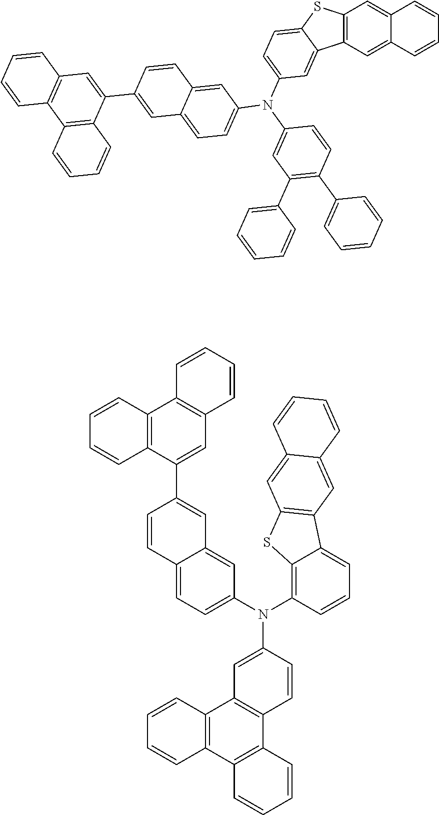 [SMILES: c1ccc(-c2ccc(N(c3ccc4cc(-c5cc6ccccc6c6ccccc56)ccc4c3)c3ccc4sc5cc6ccccc6cc5c4c3)cc2-c2ccccc2)cc1 ; c1ccc2cc3c(cc2c1)sc1c(N(c2ccc4ccc(-c5cc6ccccc6c6ccccc56)cc4c2)c2ccc4c5ccccc5c5ccccc5c4c2)cccc13]

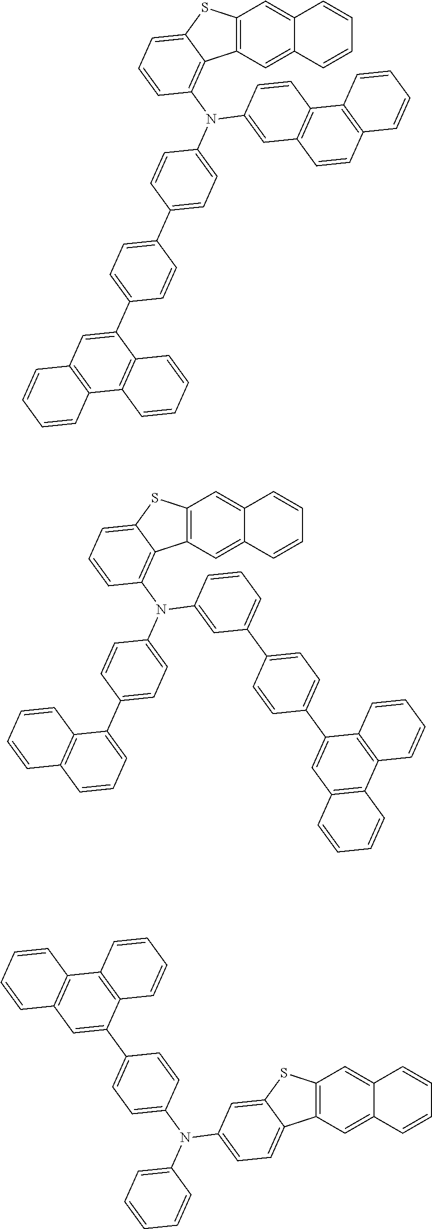 [SMILES: c1ccc2cc3c(cc2c1)sc1cccc(N(c2ccc(-c4ccc(-c5cc6ccccc6c6ccccc56)cc4)cc2)c2ccc4c(ccc5ccccc54)c2)c13 ; c1cc(-c2ccc(-c3cc4ccccc4c4ccccc34)cc2)cc(N(c2ccc(-c3cccc4ccccc34)cc2)c2cccc3sc4cc5ccccc5cc4c23)c1 ; c1ccc(N(c2ccc(-c3cc4ccccc4c4ccccc34)cc2)c2ccc3c(c2)sc2cc4ccccc4cc23)cc1]

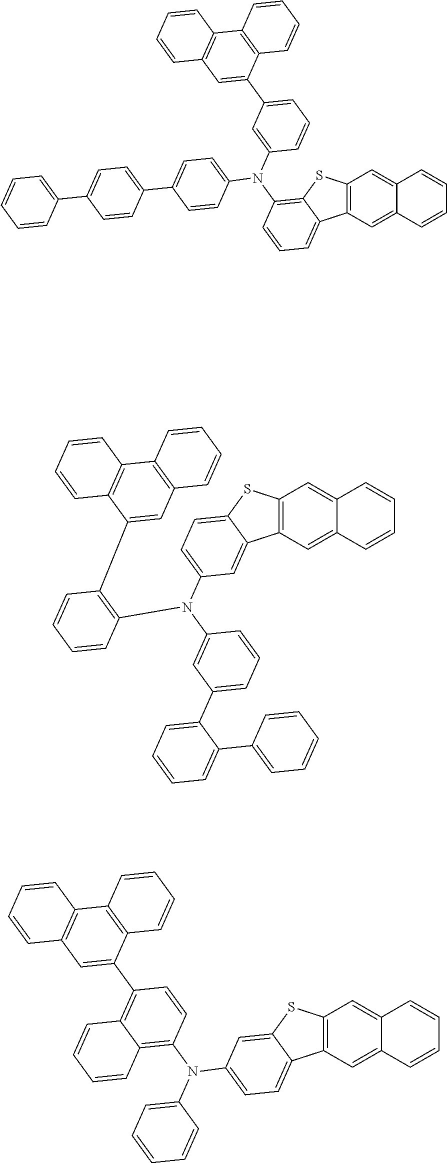 [SMILES: c1ccc(-c2ccc(-c3ccc(N(c4cccc(-c5cc6ccccc6c6ccccc56)c4)c4cccc5c4sc4cc6ccccc6cc45)cc3)cc2)cc1 ; c1ccc(-c2ccccc2-c2cccc(N(c3ccc4sc5cc6ccccc6cc5c4c3)c3ccccc3-c3cc4ccccc4c4ccccc34)c2)cc1 ; c1ccc(N(c2ccc3c(c2)sc2cc4ccccc4cc23)c2ccc(-c3cc4ccccc4c4ccccc34)c3ccccc23)cc1]

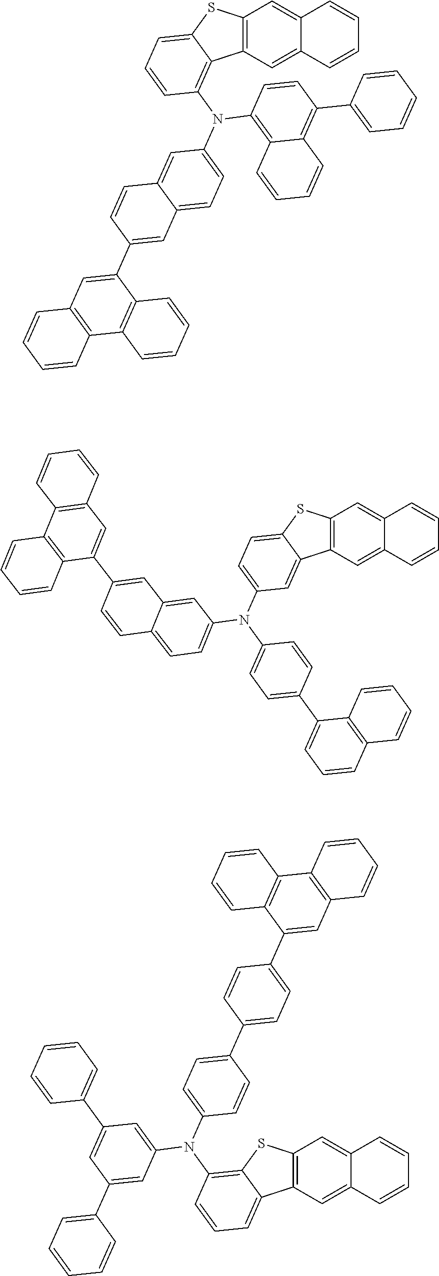 [SMILES: c1ccc(-c2ccc(N(c3ccc4cc(-c5cc6ccccc6c6ccccc56)ccc4c3)c3cccc4sc5cc6ccccc6cc5c34)c3ccccc23)cc1 ; c1ccc2cc3c(cc2c1)sc1ccc(N(c2ccc(-c4cccc5ccccc45)cc2)c2ccc4ccc(-c5cc6ccccc6c6ccccc56)cc4c2)cc13 ; c1ccc(-c2cc(-c3ccccc3)cc(N(c3ccc(-c4ccc(-c5cc6ccccc6c6ccccc56)cc4)cc3)c3cccc4c3sc3cc5ccccc5cc34)c2)cc1]

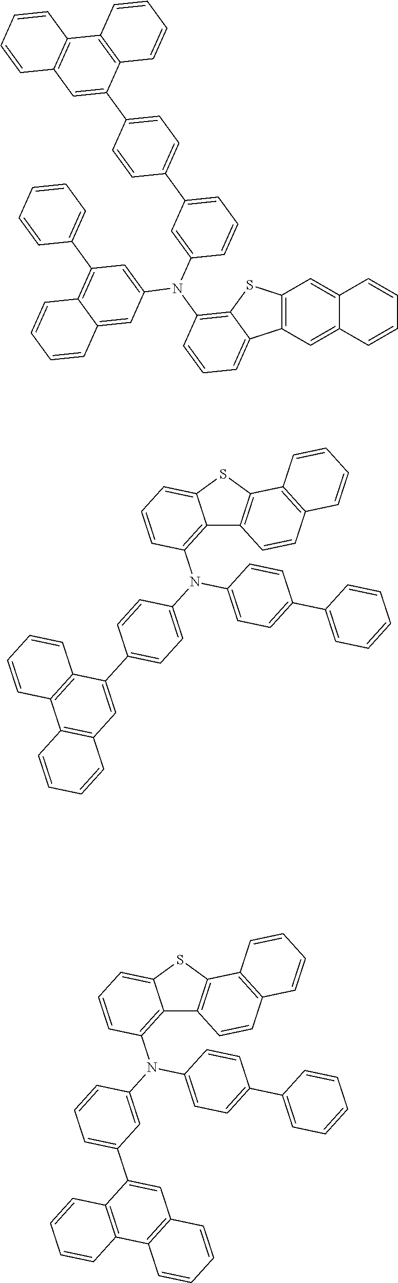 [SMILES: c1ccc(-c2cc(N(c3cccc(-c4ccc(-c5cc6ccccc6c6ccccc56)cc4)c3)c3cccc4c3sc3cc5ccccc5cc34)cc3ccccc23)cc1 ; c1ccc(-c2ccc(N(c3ccc(-c4cc5ccccc5c5ccccc45)cc3)c3cccc4sc5c6ccccc6ccc5c34)cc2)cc1 ; c1ccc(-c2ccc(N(c3cccc(-c4cc5ccccc5c5ccccc45)c3)c3cccc4sc5c6ccccc6ccc5c34)cc2)cc1]

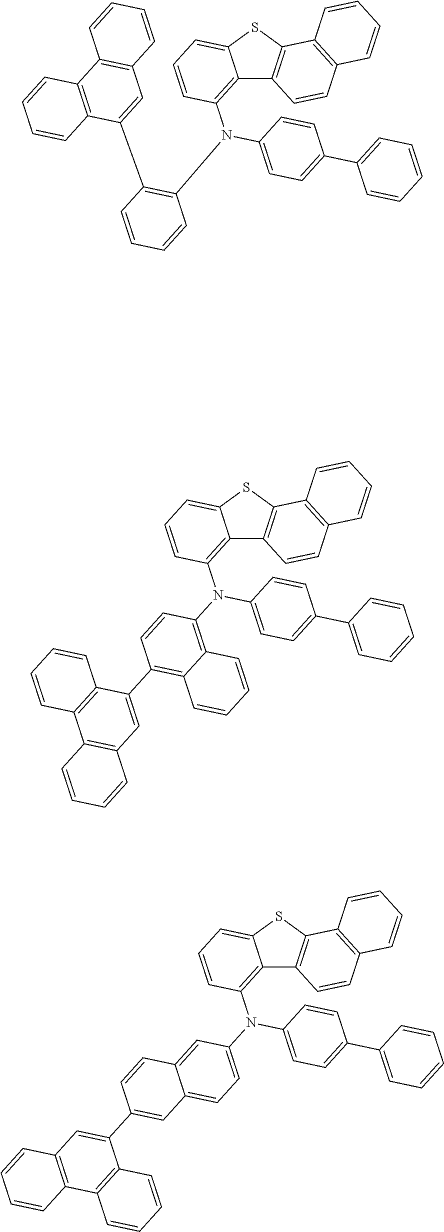 [SMILES: c1ccc(-c2ccc(N(c3ccccc3-c3cc4ccccc4c4ccccc34)c3cccc4sc5c6ccccc6ccc5c34)cc2)cc1 ; c1ccc(-c2ccc(N(c3ccc(-c4cc5ccccc5c5ccccc45)c4ccccc34)c3cccc4sc5c6ccccc6ccc5c34)cc2)cc1 ; c1ccc(-c2ccc(N(c3ccc4cc(-c5cc6ccccc6c6ccccc56)ccc4c3)c3cccc4sc5c6ccccc6ccc5c34)cc2)cc1]

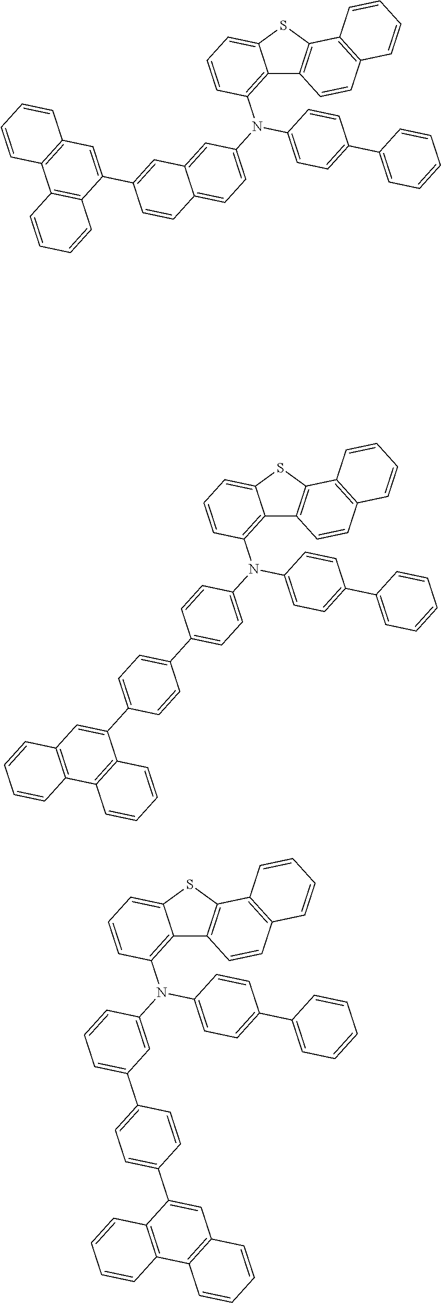 [SMILES: c1ccc(-c2ccc(N(c3ccc4ccc(-c5cc6ccccc6c6ccccc56)cc4c3)c3cccc4sc5c6ccccc6ccc5c34)cc2)cc1 ; c1ccc(-c2ccc(N(c3ccc(-c4ccc(-c5cc6ccccc6c6ccccc56)cc4)cc3)c3cccc4sc5c6ccccc6ccc5c34)cc2)cc1 ; c1ccc(-c2ccc(N(c3cccc(-c4ccc(-c5cc6ccccc6c6ccccc56)cc4)c3)c3cccc4sc5c6ccccc6ccc5c34)cc2)cc1]

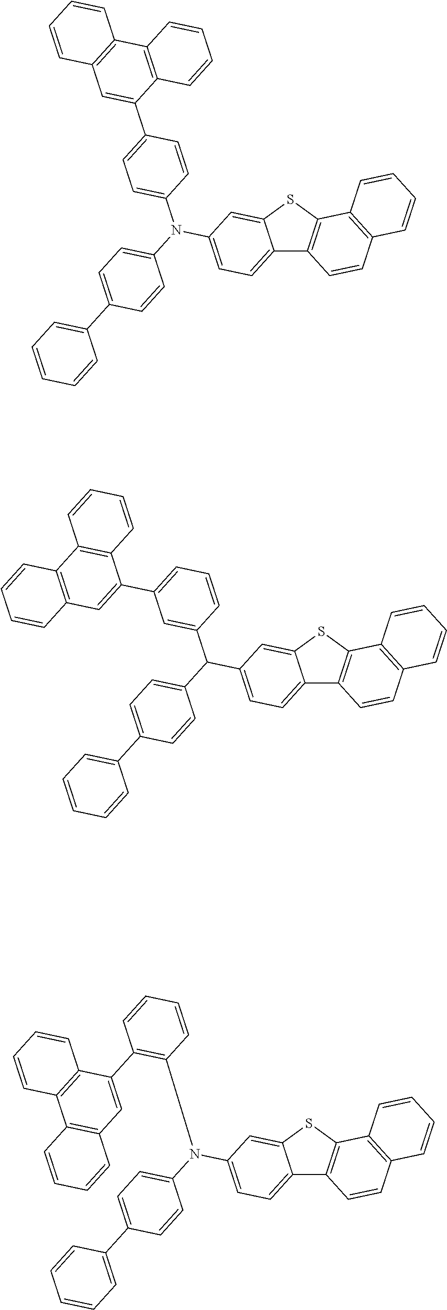 [SMILES: c1ccc(-c2ccc(N(c3ccc(-c4cc5ccccc5c5ccccc45)cc3)c3ccc4c(c3)sc3c5ccccc5ccc43)cc2)cc1 ; c1ccc(-c2ccc(C(c3cccc(-c4cc5ccccc5c5ccccc45)c3)c3ccc4c(c3)sc3c5ccccc5ccc43)cc2)cc1 ; c1ccc(-c2ccc(N(c3ccc4c(c3)sc3c5ccccc5ccc43)c3ccccc3-c3cc4ccccc4c4ccccc34)cc2)cc1]

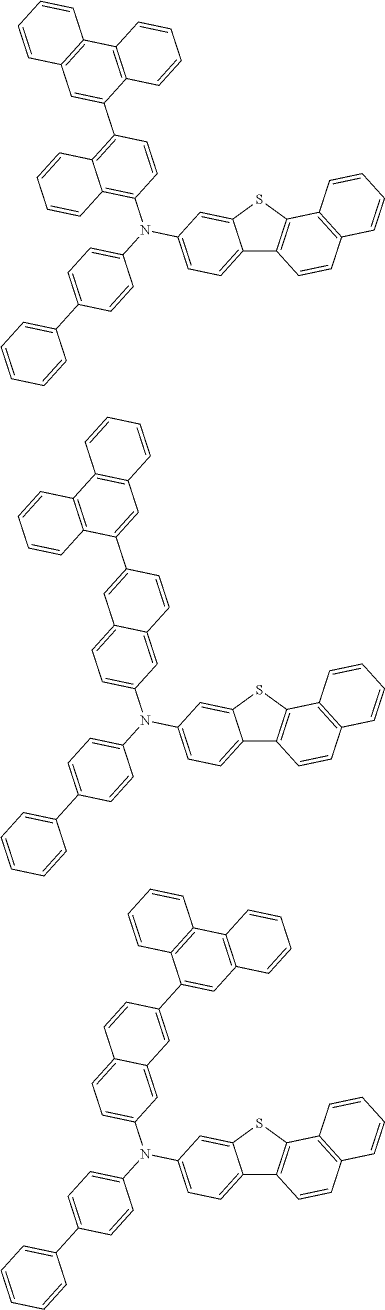 [SMILES: c1ccc(-c2ccc(N(c3ccc4c(c3)sc3c5ccccc5ccc43)c3ccc(-c4cc5ccccc5c5ccccc45)c4ccccc34)cc2)cc1 ; c1ccc(-c2ccc(N(c3ccc4cc(-c5cc6ccccc6c6ccccc56)ccc4c3)c3ccc4c(c3)sc3c5ccccc5ccc43)cc2)cc1 ; c1ccc(-c2ccc(N(c3ccc4ccc(-c5cc6ccccc6c6ccccc56)cc4c3)c3ccc4c(c3)sc3c5ccccc5ccc43)cc2)cc1]

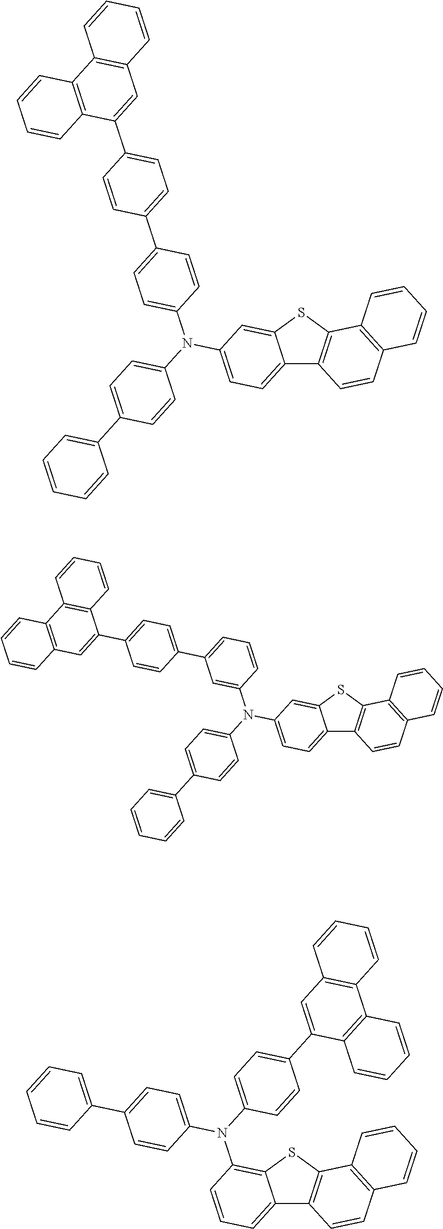 [SMILES: c1ccc(-c2ccc(N(c3ccc(-c4ccc(-c5cc6ccccc6c6ccccc56)cc4)cc3)c3ccc4c(c3)sc3c5ccccc5ccc43)cc2)cc1 ; c1ccc(-c2ccc(N(c3cccc(-c4ccc(-c5cc6ccccc6c6ccccc56)cc4)c3)c3ccc4c(c3)sc3c5ccccc5ccc43)cc2)cc1 ; c1ccc(-c2ccc(N(c3ccc(-c4cc5ccccc5c5ccccc45)cc3)c3cccc4c3sc3c5ccccc5ccc43)cc2)cc1]

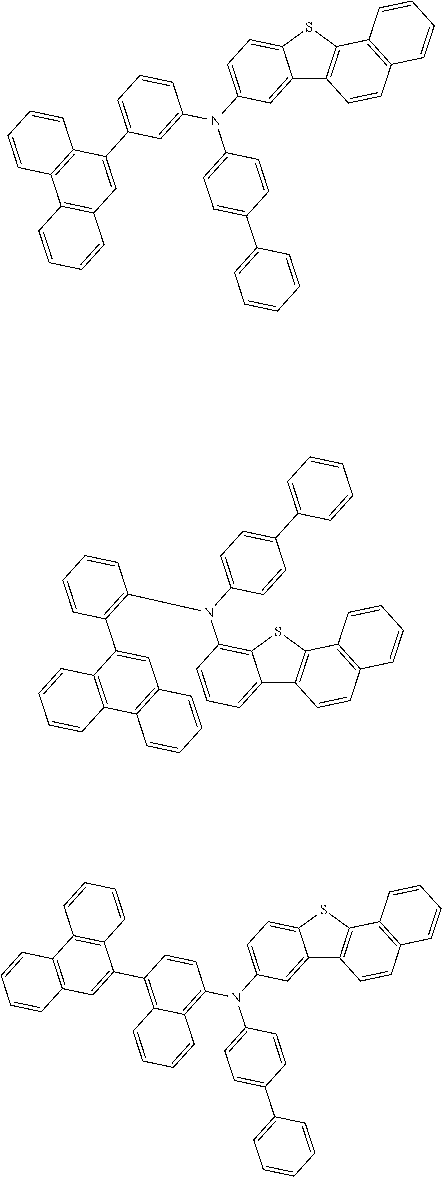 [SMILES: c1ccc(-c2ccc(N(c3cccc(-c4cc5ccccc5c5ccccc45)c3)c3ccc4sc5c6ccccc6ccc5c4c3)cc2)cc1 ; c1ccc(-c2ccc(N(c3ccccc3-c3cc4ccccc4c4ccccc34)c3cccc4c3sc3c5ccccc5ccc43)cc2)cc1 ; c1ccc(-c2ccc(N(c3ccc4sc5c6ccccc6ccc5c4c3)c3ccc(-c4cc5ccccc5c5ccccc45)c4ccccc34)cc2)cc1]

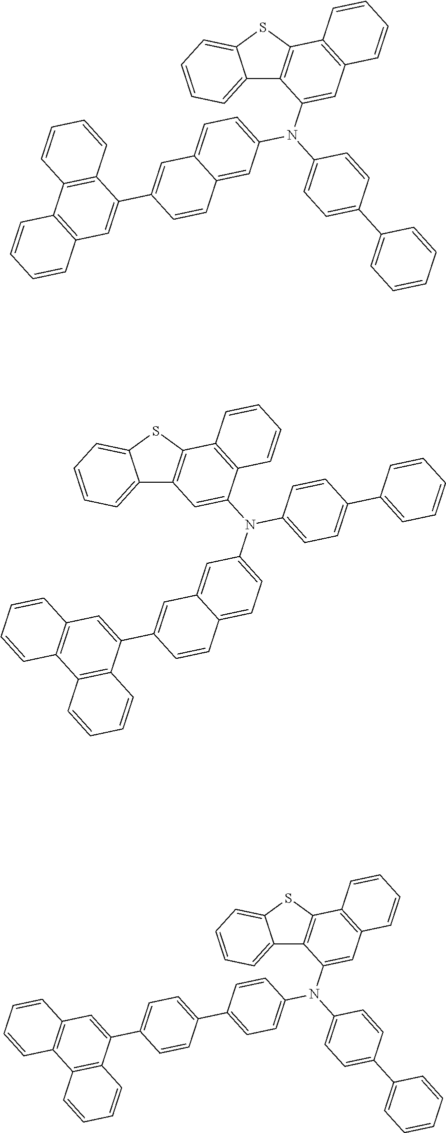 [SMILES: c1ccc(-c2ccc(N(c3ccc4cc(-c5cc6ccccc6c6ccccc56)ccc4c3)c3cc4ccccc4c4sc5ccccc5c34)cc2)cc1 ; c1ccc(-c2ccc(N(c3ccc4ccc(-c5cc6ccccc6c6ccccc56)cc4c3)c3cc4c5ccccc5sc4c4ccccc34)cc2)cc1 ; c1ccc(-c2ccc(N(c3ccc(-c4ccc(-c5cc6ccccc6c6ccccc56)cc4)cc3)c3cc4ccccc4c4sc5ccccc5c34)cc2)cc1]

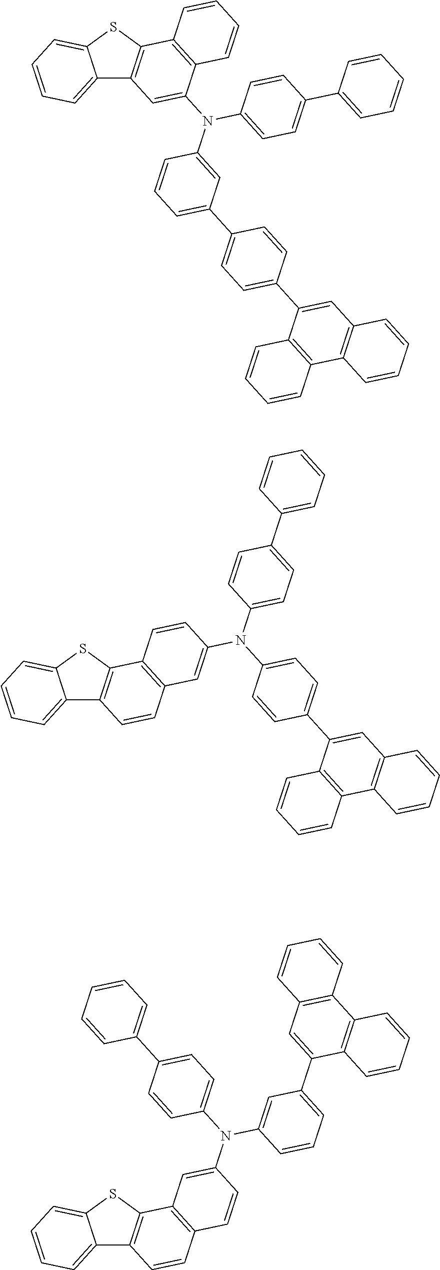 [SMILES: c1ccc(-c2ccc(N(c3cccc(-c4ccc(-c5cc6ccccc6c6ccccc56)cc4)c3)c3cc4c5ccccc5sc4c4ccccc34)cc2)cc1 ; c1ccc(-c2ccc(N(c3ccc(-c4cc5ccccc5c5ccccc45)cc3)c3ccc4c(ccc5c6ccccc6sc45)c3)cc2)cc1 ; c1ccc(-c2ccc(N(c3cccc(-c4cc5ccccc5c5ccccc45)c3)c3ccc4ccc5c6ccccc6sc5c4c3)cc2)cc1]

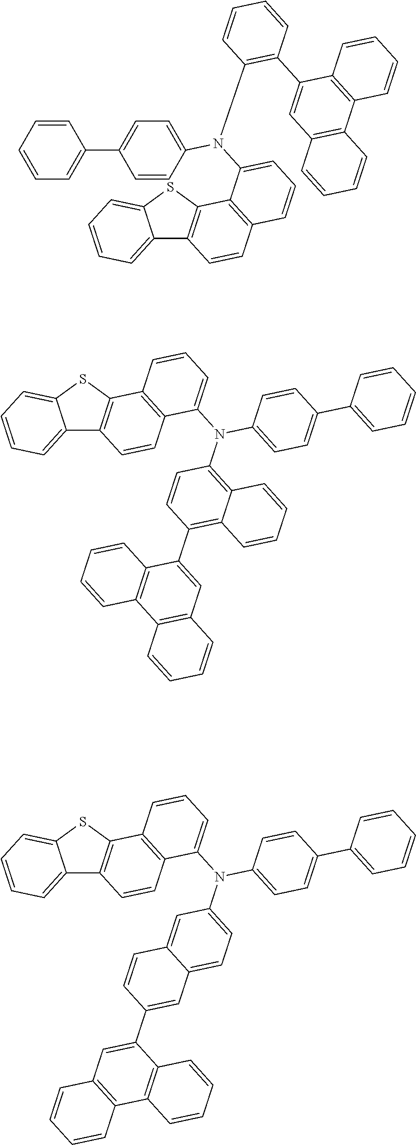 [SMILES: c1ccc(-c2ccc(N(c3ccccc3-c3cc4ccccc4c4ccccc34)c3cccc4ccc5c6ccccc6sc5c34)cc2)cc1 ; c1ccc(-c2ccc(N(c3ccc(-c4cc5ccccc5c5ccccc45)c4ccccc34)c3cccc4c3ccc3c5ccccc5sc43)cc2)cc1 ; c1ccc(-c2ccc(N(c3ccc4cc(-c5cc6ccccc6c6ccccc56)ccc4c3)c3cccc4c3ccc3c5ccccc5sc43)cc2)cc1]

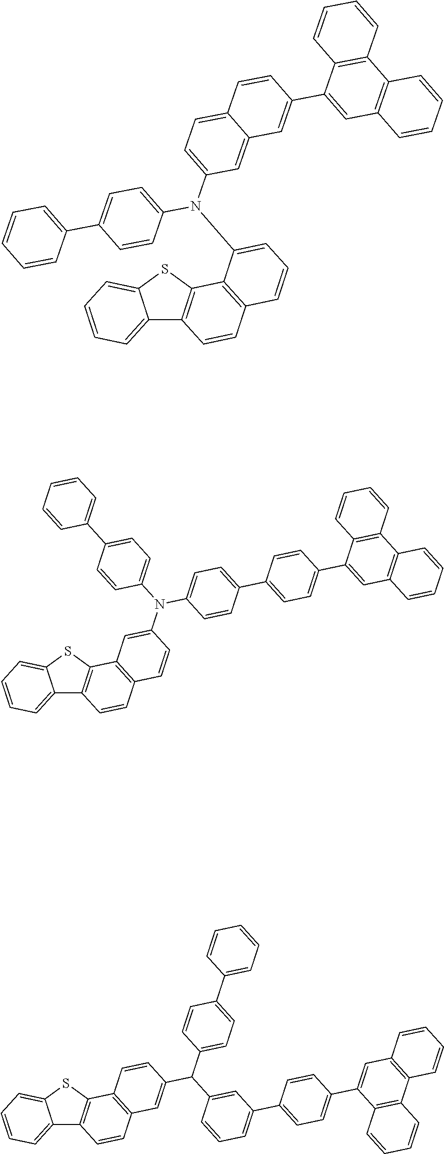 [SMILES: c1ccc(-c2ccc(N(c3ccc4ccc(-c5cc6ccccc6c6ccccc56)cc4c3)c3cccc4ccc5c6ccccc6sc5c34)cc2)cc1 ; c1ccc(-c2ccc(N(c3ccc(-c4ccc(-c5cc6ccccc6c6ccccc56)cc4)cc3)c3ccc4ccc5c6ccccc6sc5c4c3)cc2)cc1 ; c1ccc(-c2ccc(C(c3cccc(-c4ccc(-c5cc6ccccc6c6ccccc56)cc4)c3)c3ccc4c(ccc5c6ccccc6sc45)c3)cc2)cc1]

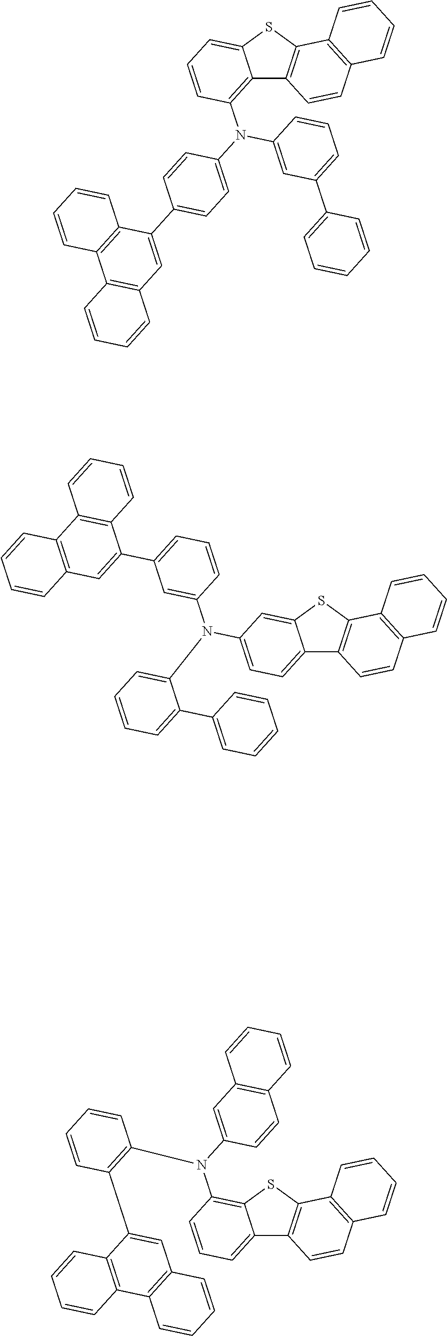 [SMILES: c1ccc(-c2cccc(N(c3ccc(-c4cc5ccccc5c5ccccc45)cc3)c3cccc4sc5c6ccccc6ccc5c34)c2)cc1 ; c1ccc(-c2ccccc2N(c2cccc(-c3cc4ccccc4c4ccccc34)c2)c2ccc3c(c2)sc2c4ccccc4ccc32)cc1 ; c1ccc(N(c2ccc3ccccc3c2)c2cccc3c2sc2c4ccccc4ccc32)c(-c2cc3ccccc3c3ccccc23)c1]

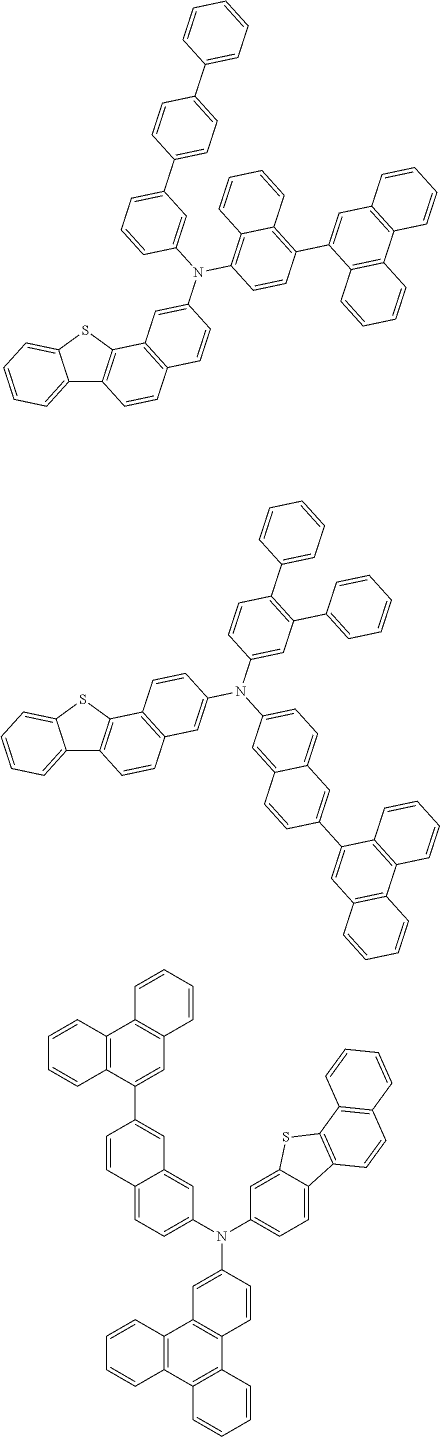 [SMILES: c1ccc(-c2ccc(-c3cccc(N(c4ccc5ccc6c7ccccc7sc6c5c4)c4ccc(-c5cc6ccccc6c6ccccc56)c5ccccc45)c3)cc2)cc1 ; c1ccc(-c2ccc(N(c3ccc4cc(-c5cc6ccccc6c6ccccc56)ccc4c3)c3ccc4c(ccc5c6ccccc6sc45)c3)cc2-c2ccccc2)cc1 ; c1ccc2c(c1)cc(-c1ccc3ccc(N(c4ccc5c(c4)sc4c6ccccc6ccc54)c4ccc5c6ccccc6c6ccccc6c5c4)cc3c1)c1ccccc12]

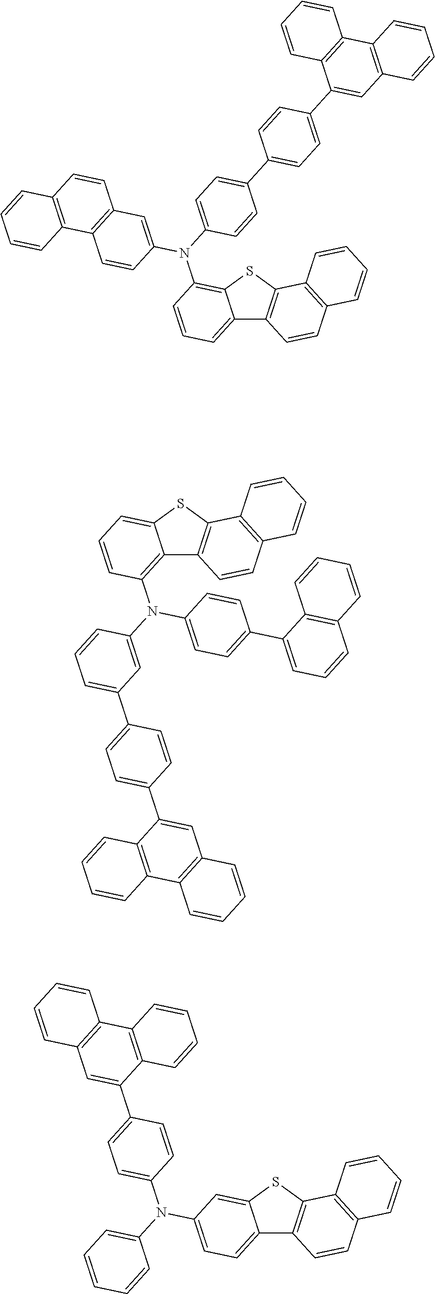 [SMILES: c1ccc2c(c1)ccc1cc(N(c3ccc(-c4ccc(-c5cc6ccccc6c6ccccc56)cc4)cc3)c3cccc4c3sc3c5ccccc5ccc43)ccc12 ; c1cc(-c2ccc(-c3cc4ccccc4c4ccccc34)cc2)cc(N(c2ccc(-c3cccc4ccccc34)cc2)c2cccc3sc4c5ccccc5ccc4c23)c1 ; c1ccc(N(c2ccc(-c3cc4ccccc4c4ccccc34)cc2)c2ccc3c(c2)sc2c4ccccc4ccc32)cc1]

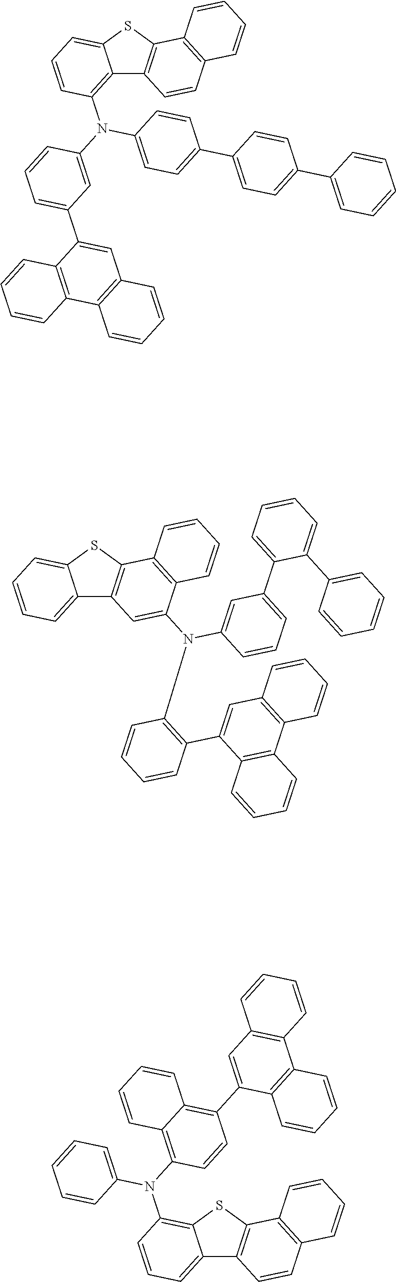 [SMILES: c1ccc(-c2ccc(-c3ccc(N(c4cccc(-c5cc6ccccc6c6ccccc56)c4)c4cccc5sc6c7ccccc7ccc6c45)cc3)cc2)cc1 ; c1ccc(-c2ccccc2-c2cccc(N(c3ccccc3-c3cc4ccccc4c4ccccc34)c3cc4c5ccccc5sc4c4ccccc34)c2)cc1 ; c1ccc(N(c2ccc(-c3cc4ccccc4c4ccccc34)c3ccccc23)c2cccc3c2sc2c4ccccc4ccc32)cc1]

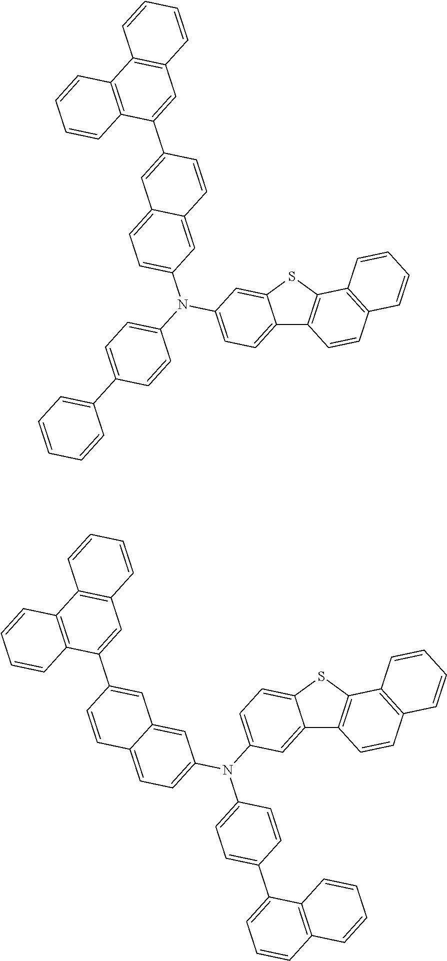 [SMILES: c1ccc(-c2ccc(N(c3ccc4cc(-c5cc6ccccc6c6ccccc56)ccc4c3)c3ccc4c(c3)sc3c5ccccc5ccc43)cc2)cc1 ; c1ccc2c(-c3ccc(N(c4ccc5ccc(-c6cc7ccccc7c7ccccc67)cc5c4)c4ccc5sc6c7ccccc7ccc6c5c4)cc3)cccc2c1]

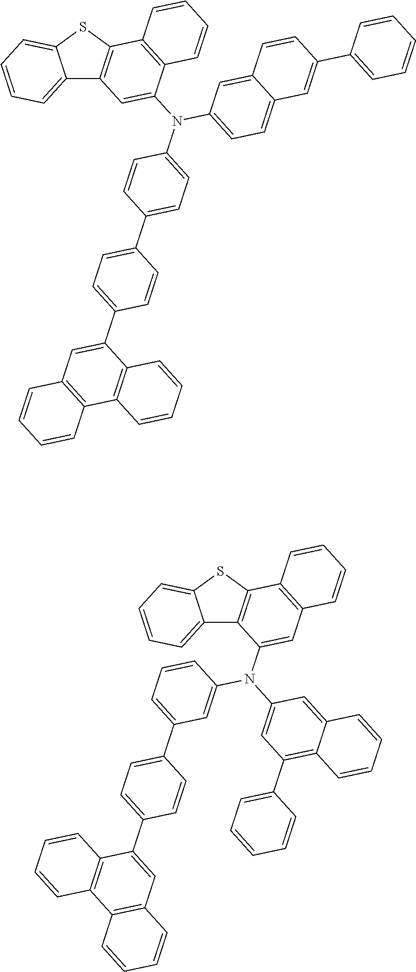 [SMILES: c1ccc(-c2ccc3cc(N(c4ccc(-c5ccc(-c6cc7ccccc7c7ccccc67)cc5)cc4)c4cc5c6ccccc6sc5c5ccccc45)ccc3c2)cc1 ; c1ccc(-c2cc(N(c3cccc(-c4ccc(-c5cc6ccccc6c6ccccc56)cc4)c3)c3cc4ccccc4c4sc5ccccc5c34)cc3ccccc23)cc1]

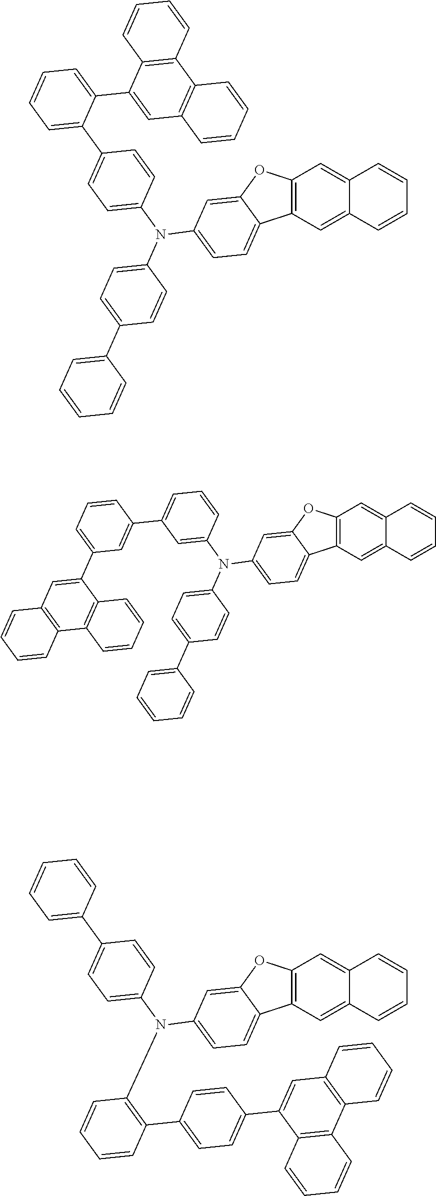 [SMILES: c1ccc(-c2ccc(N(c3ccc(-c4ccccc4-c4cc5ccccc5c5ccccc45)cc3)c3ccc4c(c3)oc3cc5ccccc5cc34)cc2)cc1 ; c1ccc(-c2ccc(N(c3cccc(-c4cccc(-c5cc6ccccc6c6ccccc56)c4)c3)c3ccc4c(c3)oc3cc5ccccc5cc34)cc2)cc1 ; c1ccc(-c2ccc(N(c3ccc4c(c3)oc3cc5ccccc5cc34)c3ccccc3-c3ccc(-c4cc5ccccc5c5ccccc45)cc3)cc2)cc1]

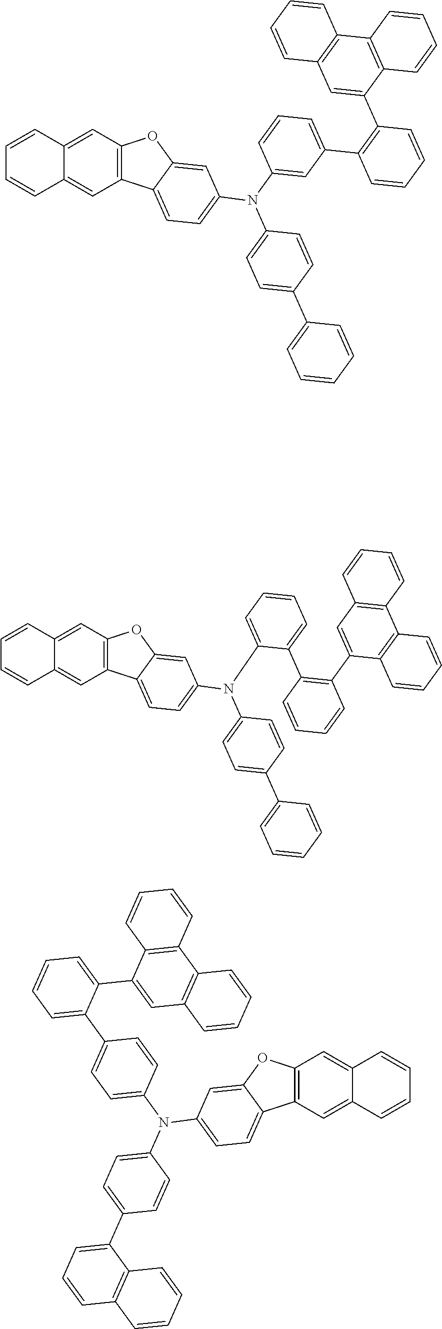 [SMILES: c1ccc(-c2ccc(N(c3cccc(-c4ccccc4-c4cc5ccccc5c5ccccc45)c3)c3ccc4c(c3)oc3cc5ccccc5cc34)cc2)cc1 ; c1ccc(-c2ccc(N(c3ccc4c(c3)oc3cc5ccccc5cc34)c3ccccc3-c3ccccc3-c3cc4ccccc4c4ccccc34)cc2)cc1 ; c1ccc(-c2cc3ccccc3c3ccccc23)c(-c2ccc(N(c3ccc(-c4cccc5ccccc45)cc3)c3ccc4c(c3)oc3cc5ccccc5cc34)cc2)c1]

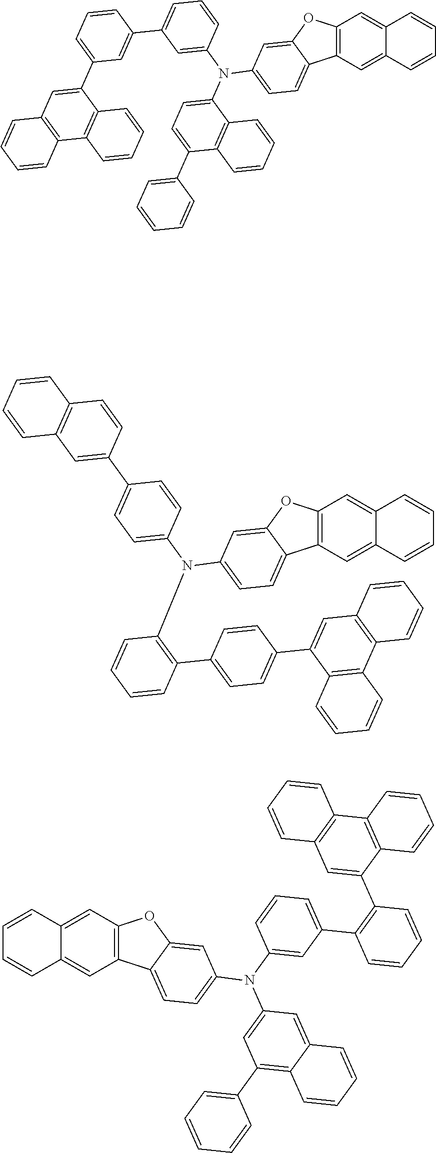 [SMILES: c1ccc(-c2ccc(N(c3cccc(-c4cccc(-c5cc6ccccc6c6ccccc56)c4)c3)c3ccc4c(c3)oc3cc5ccccc5cc34)c3ccccc23)cc1 ; c1ccc(N(c2ccc(-c3ccc4ccccc4c3)cc2)c2ccc3c(c2)oc2cc4ccccc4cc23)c(-c2ccc(-c3cc4ccccc4c4ccccc34)cc2)c1 ; c1ccc(-c2cc(N(c3cccc(-c4ccccc4-c4cc5ccccc5c5ccccc45)c3)c3ccc4c(c3)oc3cc5ccccc5cc34)cc3ccccc23)cc1]

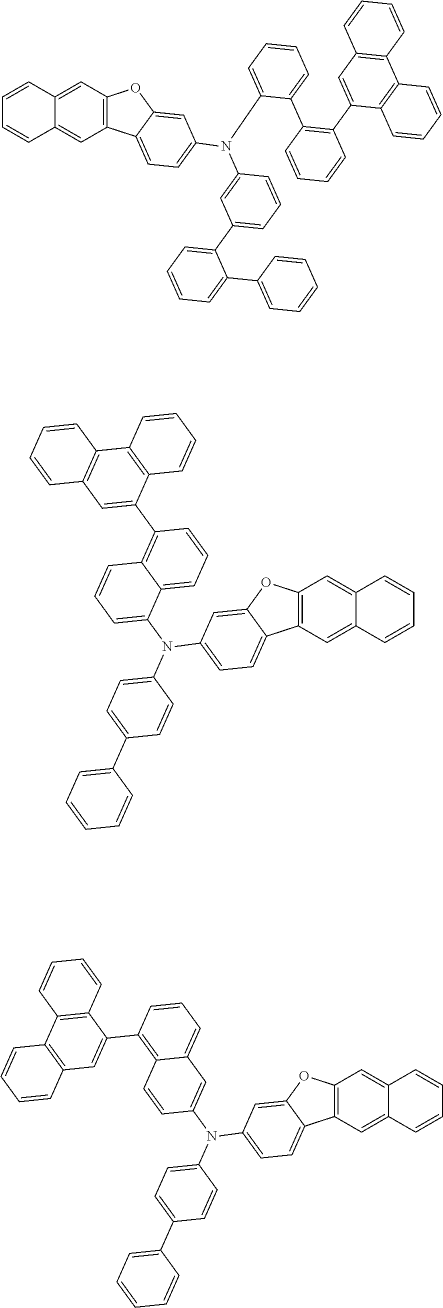 [SMILES: c1ccc(-c2ccccc2-c2cccc(N(c3ccc4c(c3)oc3cc5ccccc5cc34)c3ccccc3-c3ccccc3-c3cc4ccccc4c4ccccc34)c2)cc1 ; c1ccc(-c2ccc(N(c3ccc4c(c3)oc3cc5ccccc5cc34)c3cccc4c(-c5cc6ccccc6c6ccccc56)cccc34)cc2)cc1 ; c1ccc(-c2ccc(N(c3ccc4c(-c5cc6ccccc6c6ccccc56)cccc4c3)c3ccc4c(c3)oc3cc5ccccc5cc34)cc2)cc1]

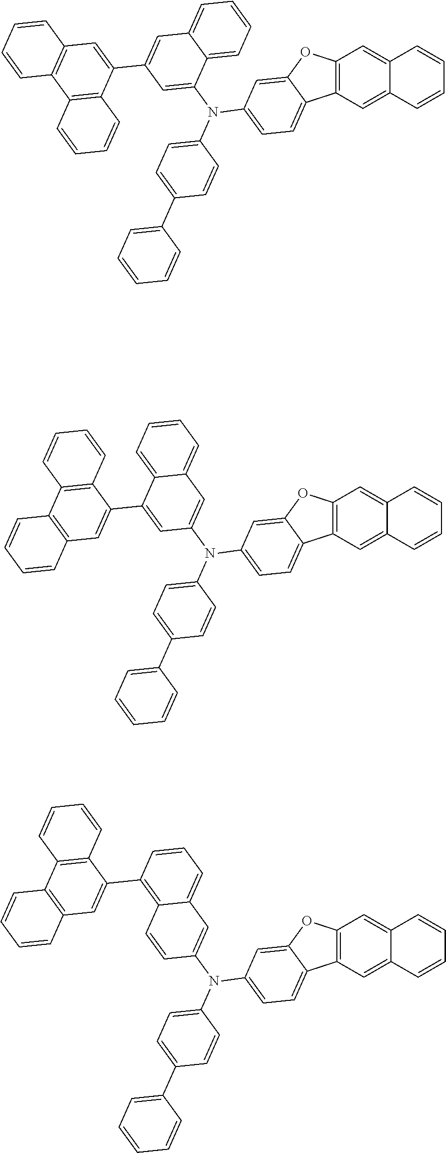 [SMILES: c1ccc(-c2ccc(N(c3ccc4c(c3)oc3cc5ccccc5cc34)c3cc(-c4cc5ccccc5c5ccccc45)cc4ccccc34)cc2)cc1 ; c1ccc(-c2ccc(N(c3cc(-c4cc5ccccc5c5ccccc45)c4ccccc4c3)c3ccc4c(c3)oc3cc5ccccc5cc34)cc2)cc1 ; c1ccc(-c2ccc(N(c3ccc4c(-c5cc6ccccc6c6ccccc56)cccc4c3)c3ccc4c(c3)oc3cc5ccccc5cc34)cc2)cc1]

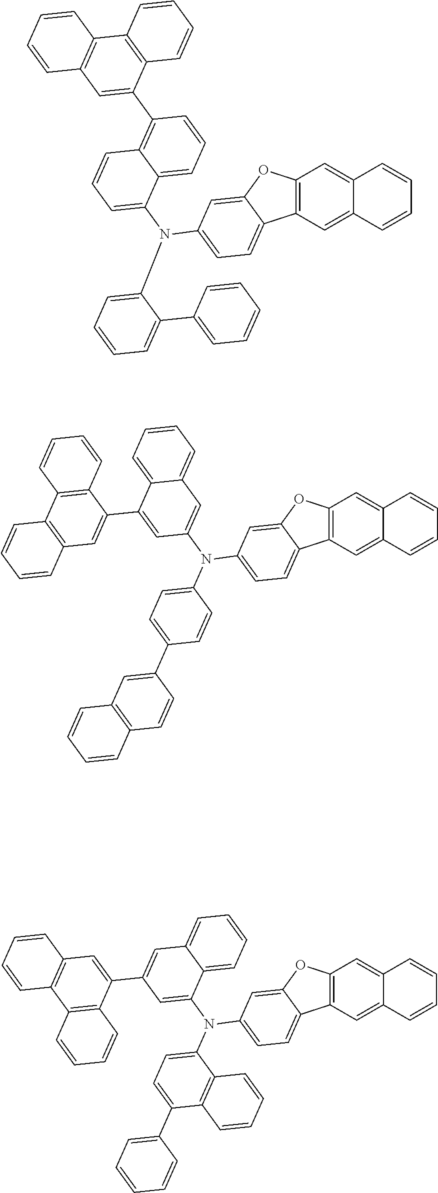 [SMILES: c1ccc(-c2ccccc2N(c2ccc3c(c2)oc2cc4ccccc4cc23)c2cccc3c(-c4cc5ccccc5c5ccccc45)cccc23)cc1 ; c1ccc2cc(-c3ccc(N(c4cc(-c5cc6ccccc6c6ccccc56)c5ccccc5c4)c4ccc5c(c4)oc4cc6ccccc6cc45)cc3)ccc2c1 ; c1ccc(-c2ccc(N(c3ccc4c(c3)oc3cc5ccccc5cc34)c3cc(-c4cc5ccccc5c5ccccc45)cc4ccccc34)c3ccccc23)cc1]

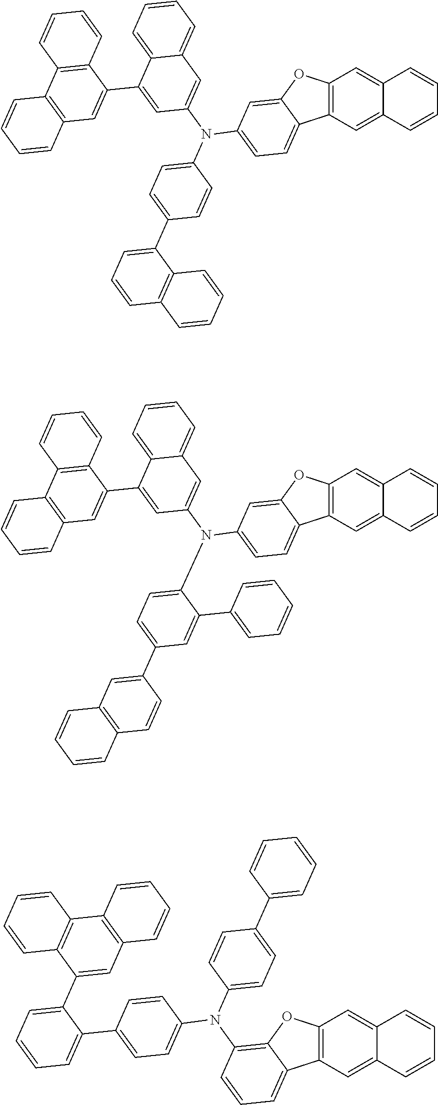 [SMILES: c1ccc2cc3c(cc2c1)oc1cc(N(c2ccc(-c4cccc5ccccc45)cc2)c2cc(-c4cc5ccccc5c5ccccc45)c4ccccc4c2)ccc13 ; c1ccc(-c2cc(-c3ccc4ccccc4c3)ccc2N(c2cc(-c3cc4ccccc4c4ccccc34)c3ccccc3c2)c2ccc3c(c2)oc2cc4ccccc4cc23)cc1 ; c1ccc(-c2ccc(N(c3ccc(-c4ccccc4-c4cc5ccccc5c5ccccc45)cc3)c3cccc4c3oc3cc5ccccc5cc34)cc2)cc1]

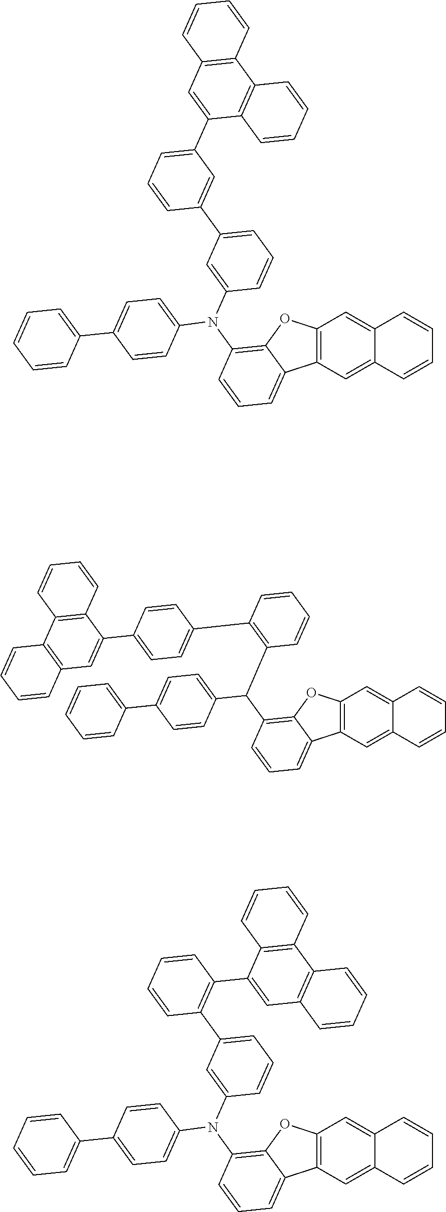 [SMILES: c1ccc(-c2ccc(N(c3cccc(-c4cccc(-c5cc6ccccc6c6ccccc56)c4)c3)c3cccc4c3oc3cc5ccccc5cc34)cc2)cc1 ; c1ccc(-c2ccc(C(c3ccccc3-c3ccc(-c4cc5ccccc5c5ccccc45)cc3)c3cccc4c3oc3cc5ccccc5cc34)cc2)cc1 ; c1ccc(-c2ccc(N(c3cccc(-c4ccccc4-c4cc5ccccc5c5ccccc45)c3)c3cccc4c3oc3cc5ccccc5cc34)cc2)cc1]

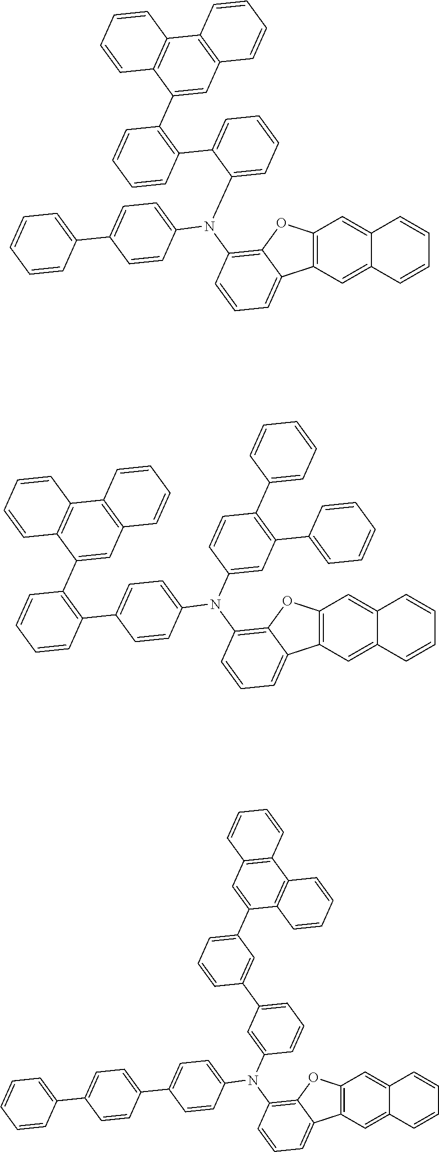 [SMILES: c1ccc(-c2ccc(N(c3ccccc3-c3ccccc3-c3cc4ccccc4c4ccccc34)c3cccc4c3oc3cc5ccccc5cc34)cc2)cc1 ; c1ccc(-c2ccc(N(c3ccc(-c4ccccc4-c4cc5ccccc5c5ccccc45)cc3)c3cccc4c3oc3cc5ccccc5cc34)cc2-c2ccccc2)cc1 ; c1ccc(-c2ccc(-c3ccc(N(c4cccc(-c5cccc(-c6cc7ccccc7c7ccccc67)c5)c4)c4cccc5c4oc4cc6ccccc6cc45)cc3)cc2)cc1]

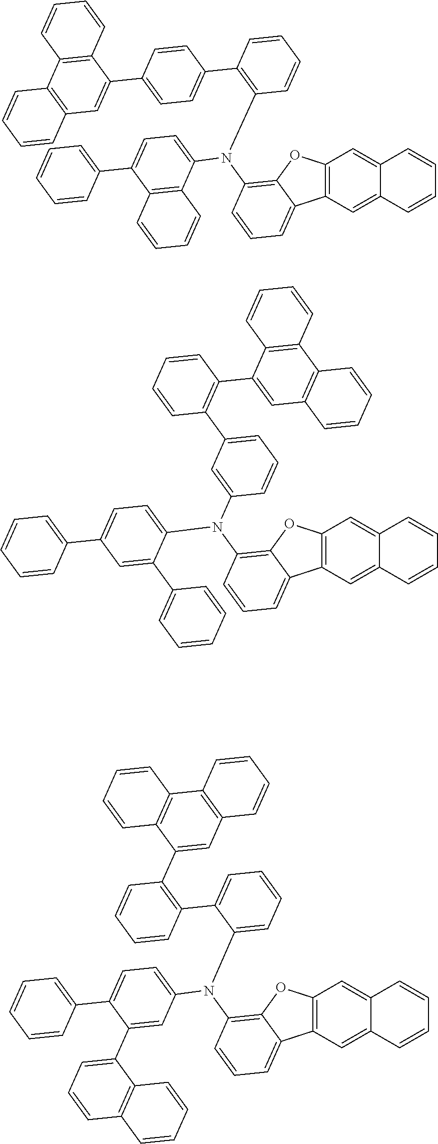 [SMILES: c1ccc(-c2ccc(N(c3ccccc3-c3ccc(-c4cc5ccccc5c5ccccc45)cc3)c3cccc4c3oc3cc5ccccc5cc34)c3ccccc23)cc1 ; c1ccc(-c2ccc(N(c3cccc(-c4ccccc4-c4cc5ccccc5c5ccccc45)c3)c3cccc4c3oc3cc5ccccc5cc34)c(-c3ccccc3)c2)cc1 ; c1ccc(-c2ccc(N(c3ccccc3-c3ccccc3-c3cc4ccccc4c4ccccc34)c3cccc4c3oc3cc5ccccc5cc34)cc2-c2cccc3ccccc23)cc1]

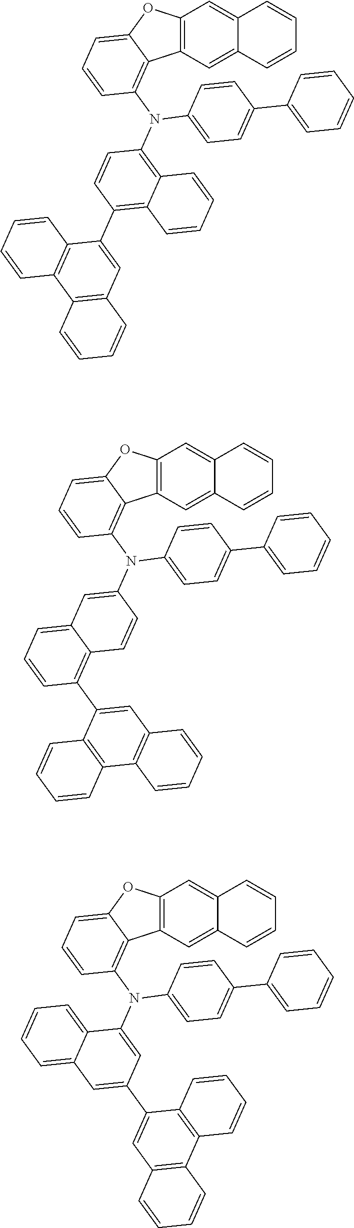 [SMILES: c1ccc(-c2ccc(N(c3ccc(-c4cc5ccccc5c5ccccc45)c4ccccc34)c3cccc4oc5cc6ccccc6cc5c34)cc2)cc1 ; c1ccc(-c2ccc(N(c3ccc4c(-c5cc6ccccc6c6ccccc56)cccc4c3)c3cccc4oc5cc6ccccc6cc5c34)cc2)cc1 ; c1ccc(-c2ccc(N(c3cc(-c4cc5ccccc5c5ccccc45)cc4ccccc34)c3cccc4oc5cc6ccccc6cc5c34)cc2)cc1]

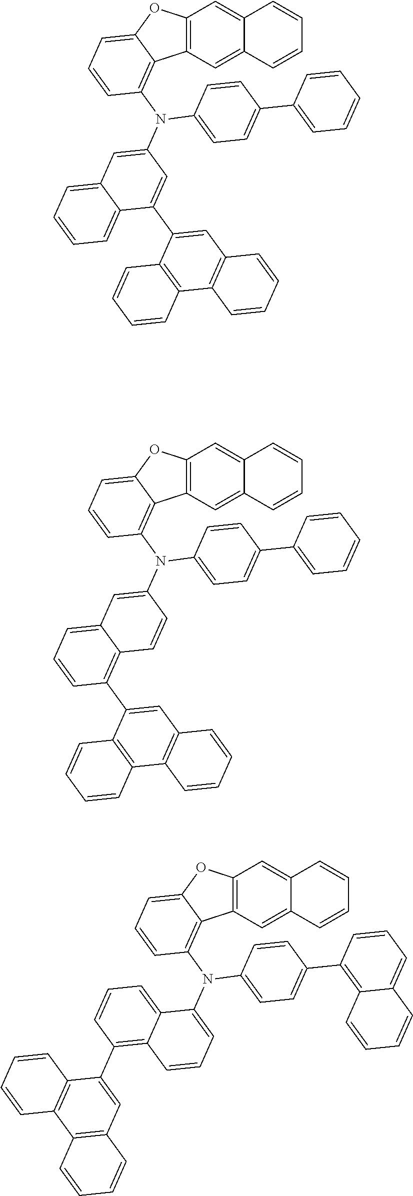 [SMILES: c1ccc(-c2ccc(N(c3cc(-c4cc5ccccc5c5ccccc45)c4ccccc4c3)c3cccc4oc5cc6ccccc6cc5c34)cc2)cc1 ; c1ccc(-c2ccc(N(c3ccc4c(-c5cc6ccccc6c6ccccc56)cccc4c3)c3cccc4oc5cc6ccccc6cc5c34)cc2)cc1 ; c1ccc2cc3c(cc2c1)oc1cccc(N(c2ccc(-c4cccc5ccccc45)cc2)c2cccc4c(-c5cc6ccccc6c6ccccc56)cccc24)c13]

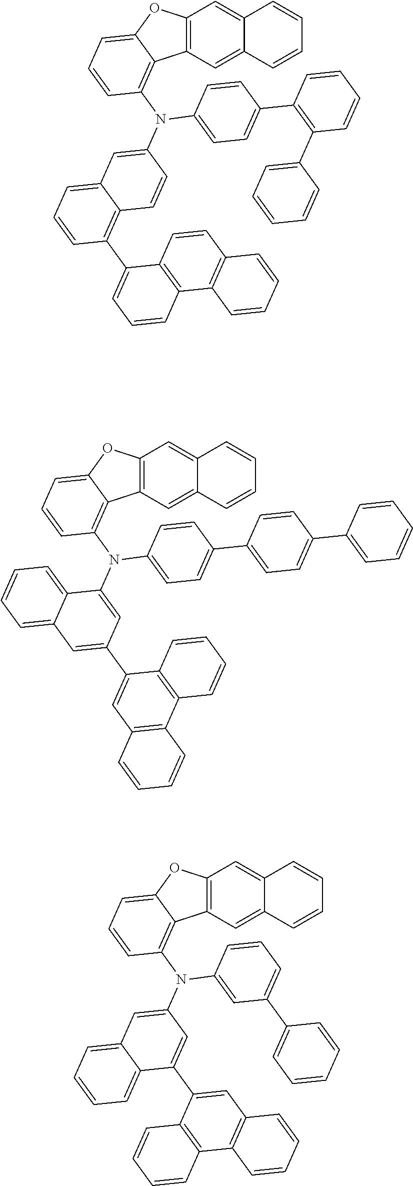 [SMILES: c1ccc(-c2ccccc2-c2ccc(N(c3ccc4c(-c5cccc6c5ccc5ccccc56)cccc4c3)c3cccc4oc5cc6ccccc6cc5c34)cc2)cc1 ; c1ccc(-c2ccc(-c3ccc(N(c4cc(-c5cc6ccccc6c6ccccc56)cc5ccccc45)c4cccc5oc6cc7ccccc7cc6c45)cc3)cc2)cc1 ; c1ccc(-c2cccc(N(c3cc(-c4cc5ccccc5c5ccccc45)c4ccccc4c3)c3cccc4oc5cc6ccccc6cc5c34)c2)cc1]

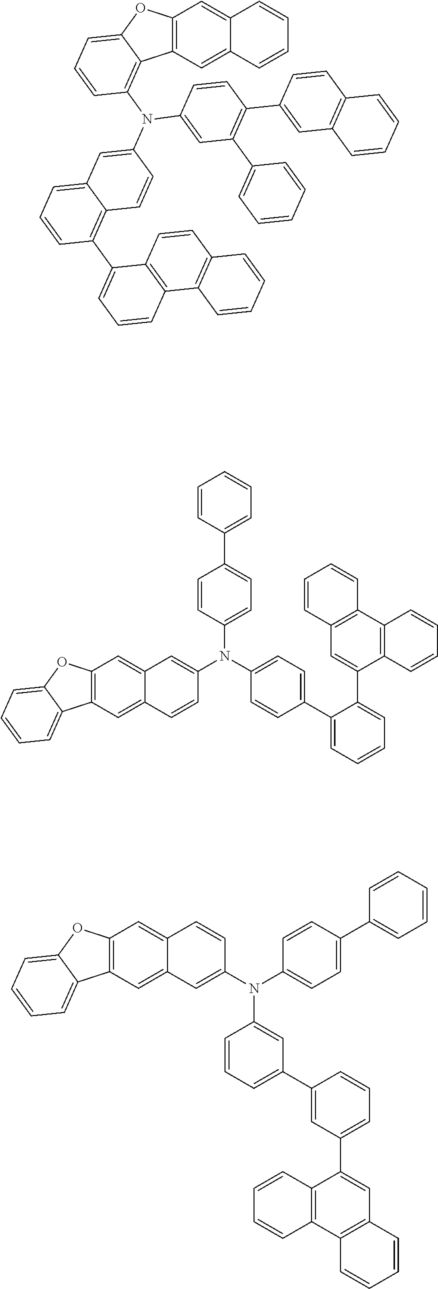 [SMILES: c1ccc(-c2cc(N(c3ccc4c(-c5cccc6c5ccc5ccccc56)cccc4c3)c3cccc4oc5cc6ccccc6cc5c34)ccc2-c2ccc3ccccc3c2)cc1 ; c1ccc(-c2ccc(N(c3ccc(-c4ccccc4-c4cc5ccccc5c5ccccc45)cc3)c3ccc4cc5c(cc4c3)oc3ccccc35)cc2)cc1 ; c1ccc(-c2ccc(N(c3cccc(-c4cccc(-c5cc6ccccc6c6ccccc56)c4)c3)c3ccc4cc5oc6ccccc6c5cc4c3)cc2)cc1]

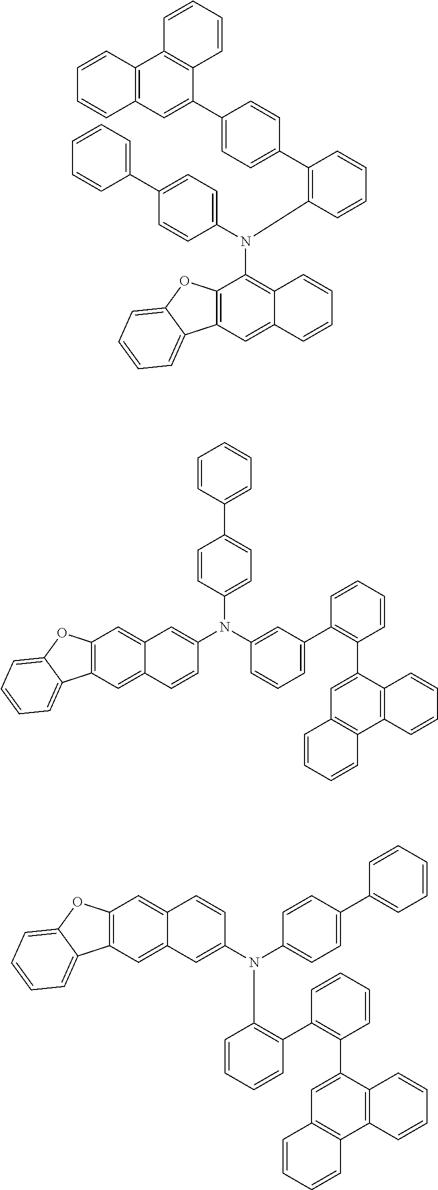 [SMILES: c1ccc(-c2ccc(N(c3ccccc3-c3ccc(-c4cc5ccccc5c5ccccc45)cc3)c3c4ccccc4cc4c3oc3ccccc34)cc2)cc1 ; c1ccc(-c2ccc(N(c3cccc(-c4ccccc4-c4cc5ccccc5c5ccccc45)c3)c3ccc4cc5c(cc4c3)oc3ccccc35)cc2)cc1 ; c1ccc(-c2ccc(N(c3ccc4cc5oc6ccccc6c5cc4c3)c3ccccc3-c3ccccc3-c3cc4ccccc4c4ccccc34)cc2)cc1]

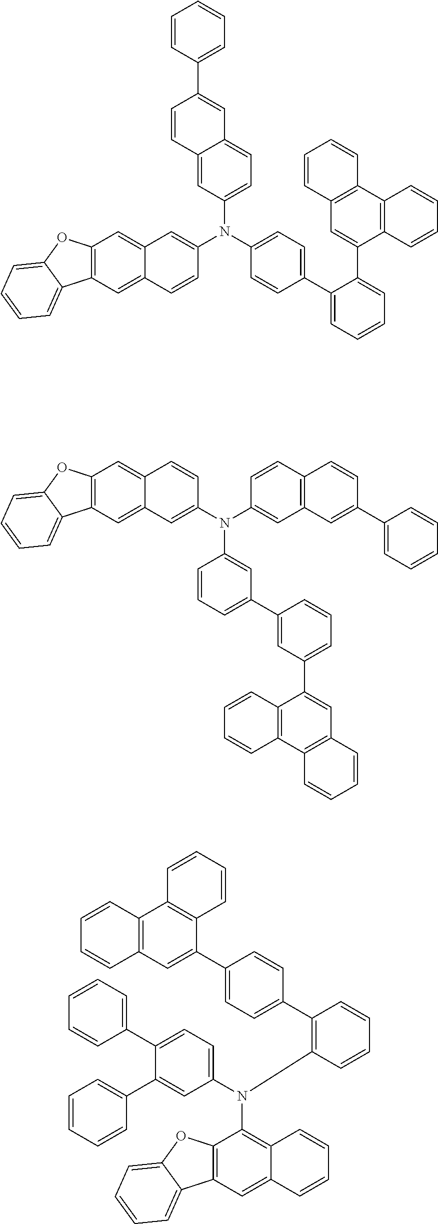 [SMILES: c1ccc(-c2ccc3cc(N(c4ccc(-c5ccccc5-c5cc6ccccc6c6ccccc56)cc4)c4ccc5cc6c(cc5c4)oc4ccccc46)ccc3c2)cc1 ; c1ccc(-c2ccc3ccc(N(c4cccc(-c5cccc(-c6cc7ccccc7c7ccccc67)c5)c4)c4ccc5cc6oc7ccccc7c6cc5c4)cc3c2)cc1 ; c1ccc(-c2ccc(N(c3ccccc3-c3ccc(-c4cc5ccccc5c5ccccc45)cc3)c3c4ccccc4cc4c3oc3ccccc34)cc2-c2ccccc2)cc1]

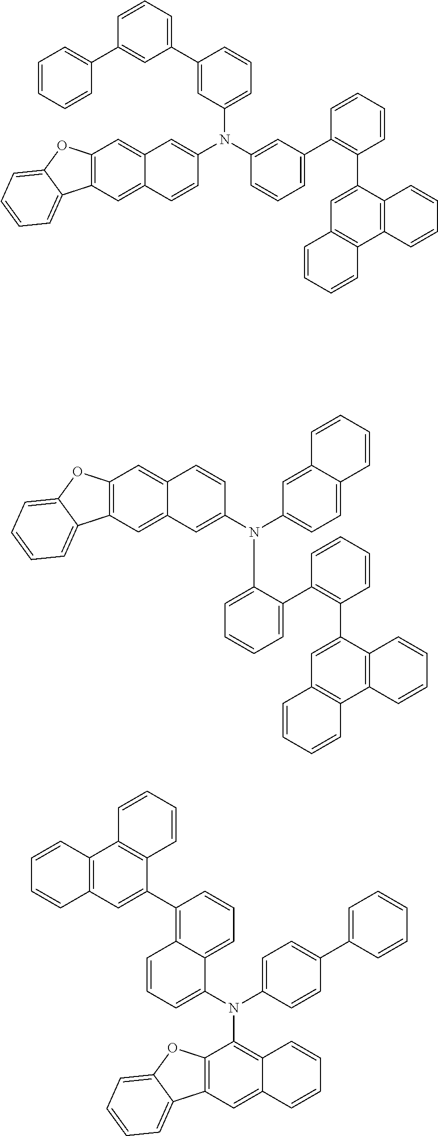 [SMILES: c1ccc(-c2cccc(-c3cccc(N(c4cccc(-c5ccccc5-c5cc6ccccc6c6ccccc56)c4)c4ccc5cc6c(cc5c4)oc4ccccc46)c3)c2)cc1 ; c1ccc(-c2cc3ccccc3c3ccccc23)c(-c2ccccc2N(c2ccc3ccccc3c2)c2ccc3cc4oc5ccccc5c4cc3c2)c1 ; c1ccc(-c2ccc(N(c3cccc4c(-c5cc6ccccc6c6ccccc56)cccc34)c3c4ccccc4cc4c3oc3ccccc34)cc2)cc1]

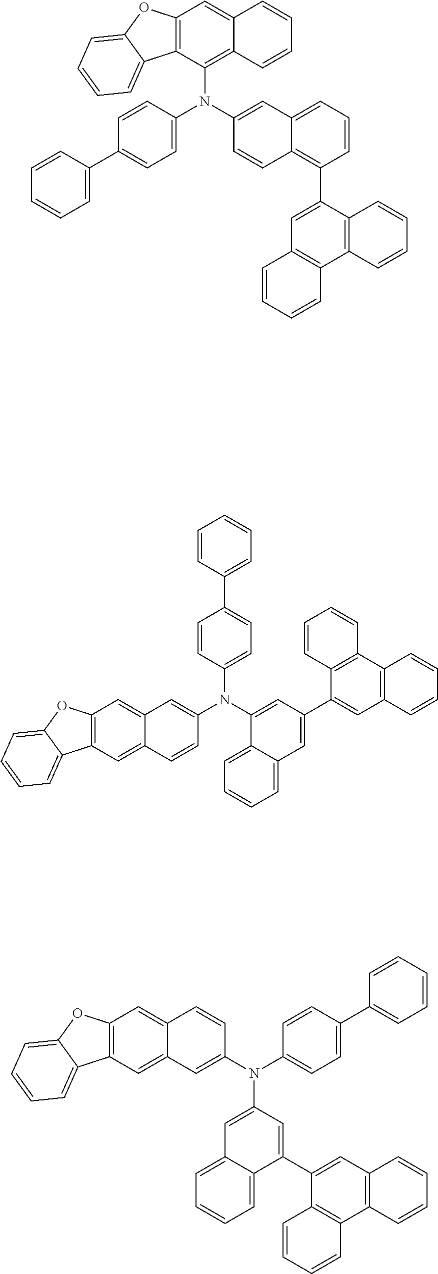 [SMILES: c1ccc(-c2ccc(N(c3ccc4c(-c5cc6ccccc6c6ccccc56)cccc4c3)c3c4ccccc4cc4oc5ccccc5c34)cc2)cc1 ; c1ccc(-c2ccc(N(c3ccc4cc5c(cc4c3)oc3ccccc35)c3cc(-c4cc5ccccc5c5ccccc45)cc4ccccc34)cc2)cc1 ; c1ccc(-c2ccc(N(c3ccc4cc5oc6ccccc6c5cc4c3)c3cc(-c4cc5ccccc5c5ccccc45)c4ccccc4c3)cc2)cc1]

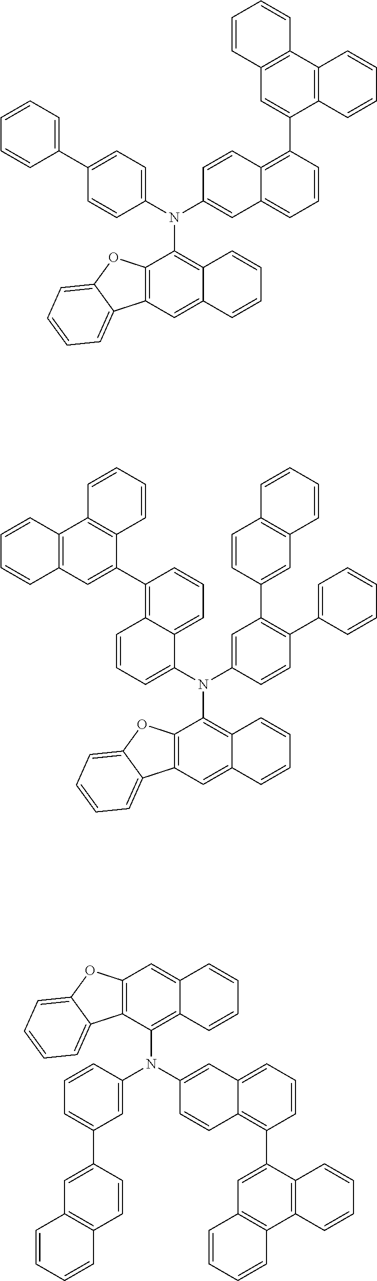 [SMILES: c1ccc(-c2ccc(N(c3ccc4c(-c5cc6ccccc6c6ccccc56)cccc4c3)c3c4ccccc4cc4c3oc3ccccc34)cc2)cc1 ; c1ccc(-c2ccc(N(c3cccc4c(-c5cc6ccccc6c6ccccc56)cccc34)c3c4ccccc4cc4c3oc3ccccc34)cc2-c2ccc3ccccc3c2)cc1 ; c1cc(-c2ccc3ccccc3c2)cc(N(c2ccc3c(-c4cc5ccccc5c5ccccc45)cccc3c2)c2c3ccccc3cc3oc4ccccc4c23)c1]

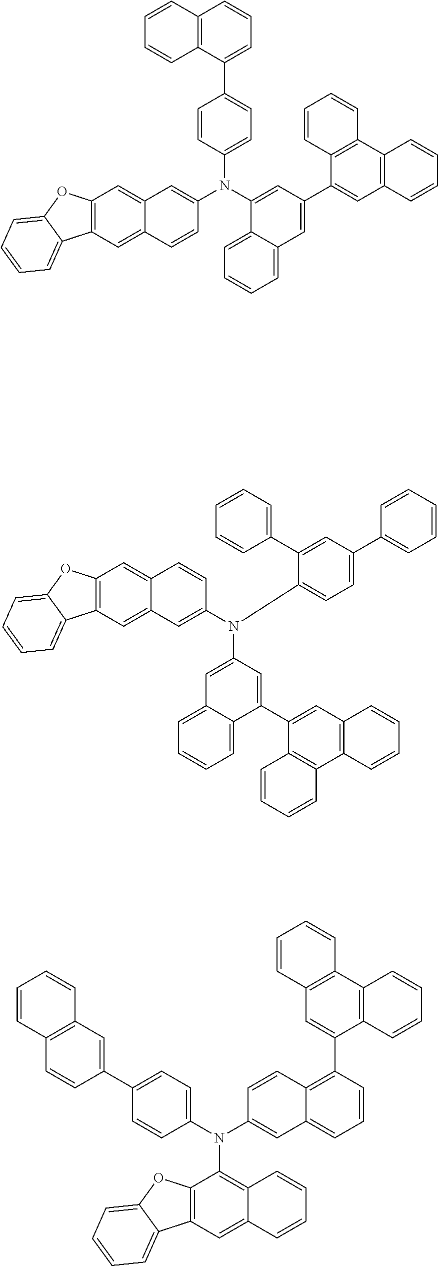 [SMILES: c1ccc2c(-c3ccc(N(c4ccc5cc6c(cc5c4)oc4ccccc46)c4cc(-c5cc6ccccc6c6ccccc56)cc5ccccc45)cc3)cccc2c1 ; c1ccc(-c2ccc(N(c3ccc4cc5oc6ccccc6c5cc4c3)c3cc(-c4cc5ccccc5c5ccccc45)c4ccccc4c3)c(-c3ccccc3)c2)cc1 ; c1ccc2cc(-c3ccc(N(c4ccc5c(-c6cc7ccccc7c7ccccc67)cccc5c4)c4c5ccccc5cc5c4oc4ccccc45)cc3)ccc2c1]

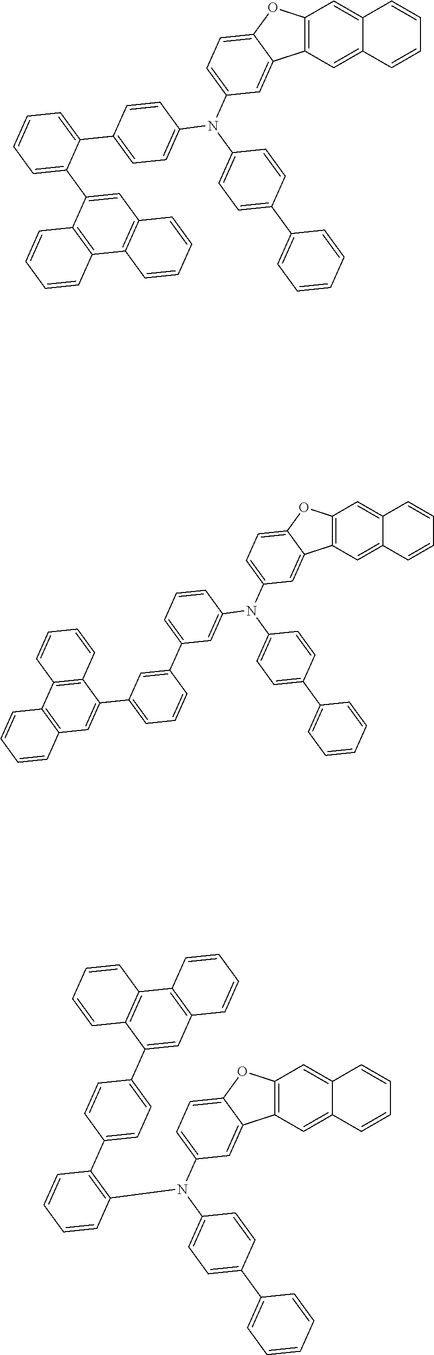 [SMILES: c1ccc(-c2ccc(N(c3ccc(-c4ccccc4-c4cc5ccccc5c5ccccc45)cc3)c3ccc4oc5cc6ccccc6cc5c4c3)cc2)cc1 ; c1ccc(-c2ccc(N(c3cccc(-c4cccc(-c5cc6ccccc6c6ccccc56)c4)c3)c3ccc4oc5cc6ccccc6cc5c4c3)cc2)cc1 ; c1ccc(-c2ccc(N(c3ccc4oc5cc6ccccc6cc5c4c3)c3ccccc3-c3ccc(-c4cc5ccccc5c5ccccc45)cc3)cc2)cc1]

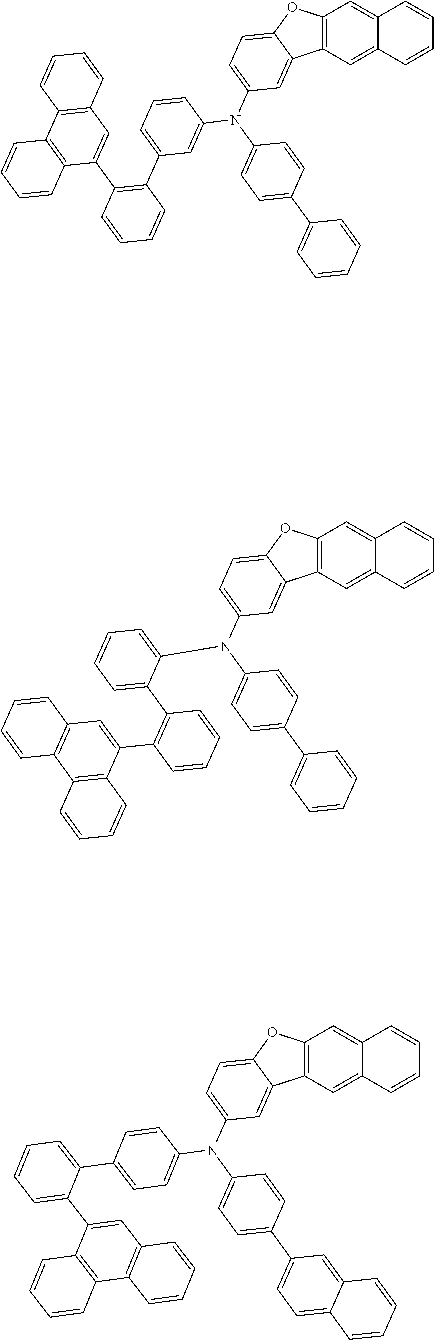 [SMILES: c1ccc(-c2ccc(N(c3cccc(-c4ccccc4-c4cc5ccccc5c5ccccc45)c3)c3ccc4oc5cc6ccccc6cc5c4c3)cc2)cc1 ; c1ccc(-c2ccc(N(c3ccc4oc5cc6ccccc6cc5c4c3)c3ccccc3-c3ccccc3-c3cc4ccccc4c4ccccc34)cc2)cc1 ; c1ccc(-c2cc3ccccc3c3ccccc23)c(-c2ccc(N(c3ccc(-c4ccc5ccccc5c4)cc3)c3ccc4oc5cc6ccccc6cc5c4c3)cc2)c1]

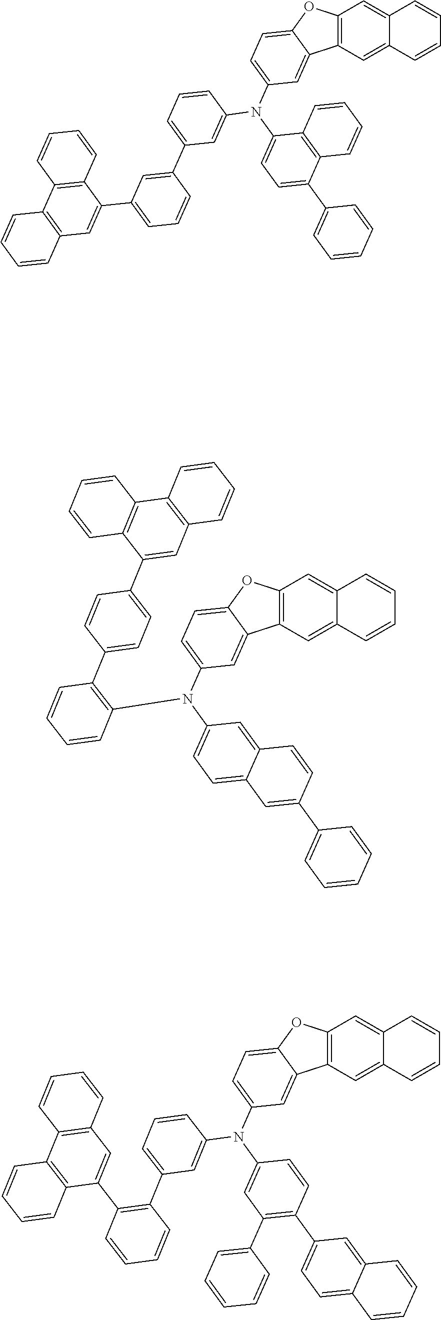 [SMILES: c1ccc(-c2ccc(N(c3cccc(-c4cccc(-c5cc6ccccc6c6ccccc56)c4)c3)c3ccc4oc5cc6ccccc6cc5c4c3)c3ccccc23)cc1 ; c1ccc(-c2ccc3cc(N(c4ccc5oc6cc7ccccc7cc6c5c4)c4ccccc4-c4ccc(-c5cc6ccccc6c6ccccc56)cc4)ccc3c2)cc1 ; c1ccc(-c2cc(N(c3cccc(-c4ccccc4-c4cc5ccccc5c5ccccc45)c3)c3ccc4oc5cc6ccccc6cc5c4c3)ccc2-c2ccc3ccccc3c2)cc1]

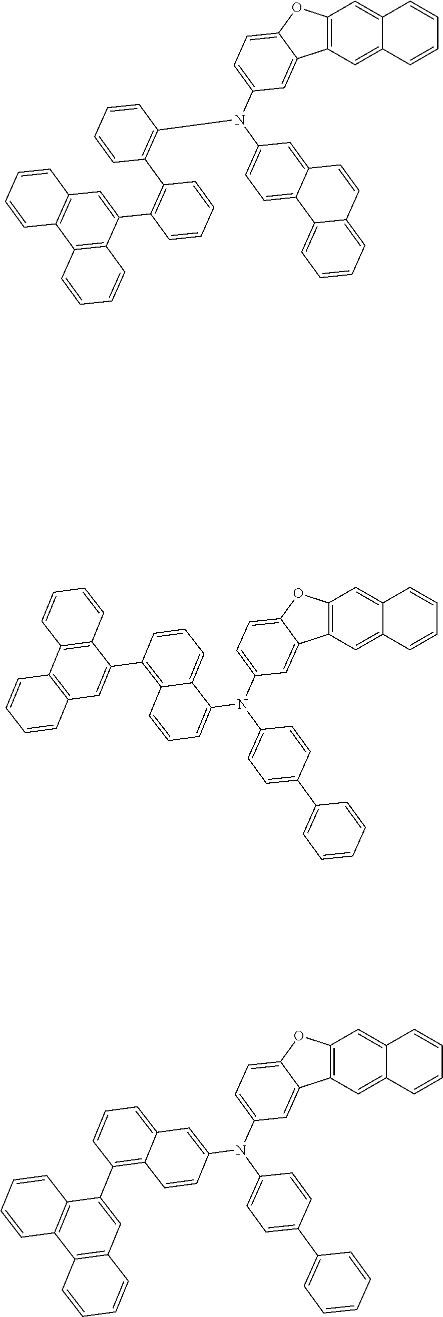 [SMILES: c1ccc(-c2cc3ccccc3c3ccccc23)c(-c2ccccc2N(c2ccc3c(ccc4ccccc43)c2)c2ccc3oc4cc5ccccc5cc4c3c2)c1 ; c1ccc(-c2ccc(N(c3ccc4oc5cc6ccccc6cc5c4c3)c3cccc4c(-c5cc6ccccc6c6ccccc56)cccc34)cc2)cc1 ; c1ccc(-c2ccc(N(c3ccc4c(-c5cc6ccccc6c6ccccc56)cccc4c3)c3ccc4oc5cc6ccccc6cc5c4c3)cc2)cc1]

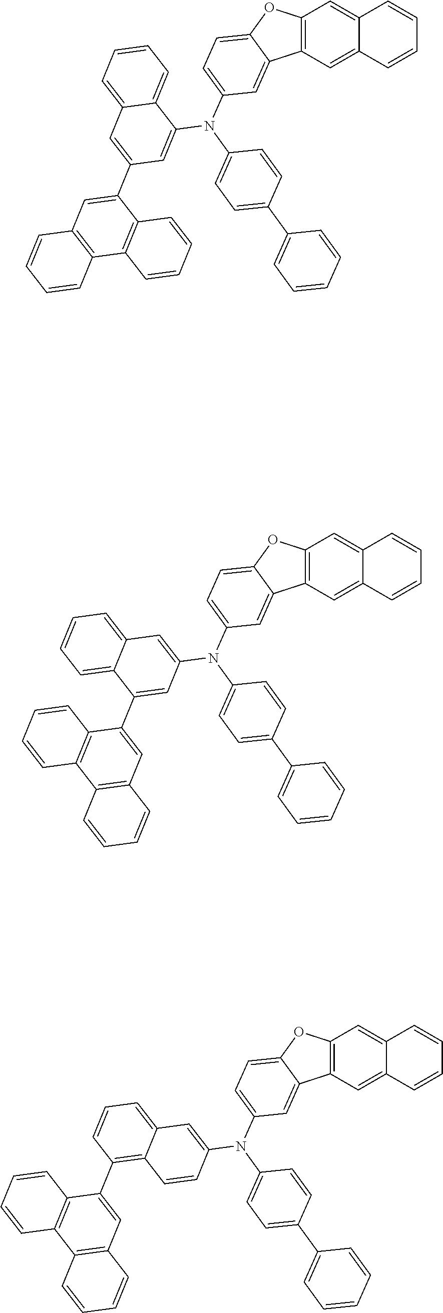 [SMILES: c1ccc(-c2ccc(N(c3ccc4oc5cc6ccccc6cc5c4c3)c3cc(-c4cc5ccccc5c5ccccc45)cc4ccccc34)cc2)cc1 ; c1ccc(-c2ccc(N(c3cc(-c4cc5ccccc5c5ccccc45)c4ccccc4c3)c3ccc4oc5cc6ccccc6cc5c4c3)cc2)cc1 ; c1ccc(-c2ccc(N(c3ccc4c(-c5cc6ccccc6c6ccccc56)cccc4c3)c3ccc4oc5cc6ccccc6cc5c4c3)cc2)cc1]

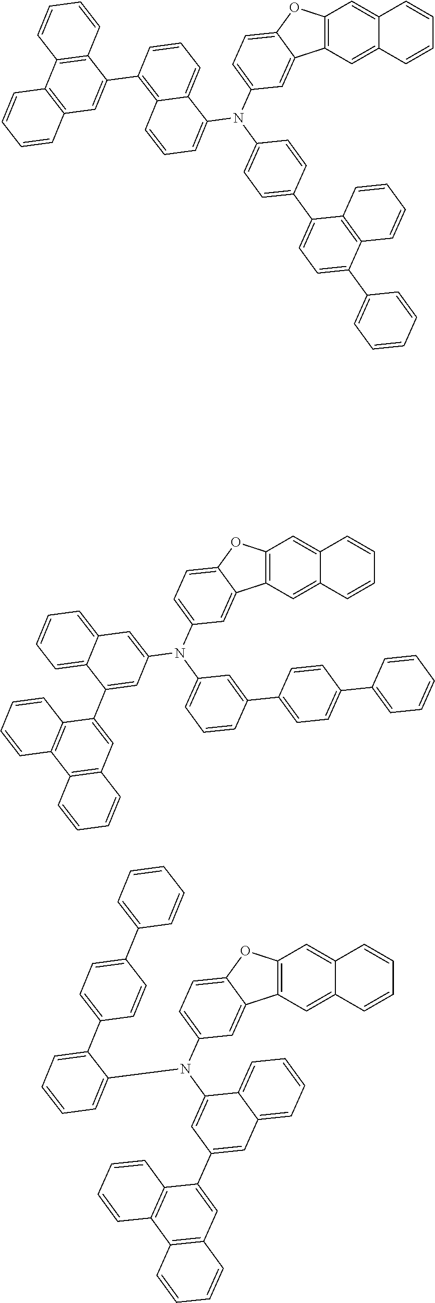 [SMILES: c1ccc(-c2ccc(-c3ccc(N(c4ccc5oc6cc7ccccc7cc6c5c4)c4cccc5c(-c6cc7ccccc7c7ccccc67)cccc45)cc3)c3ccccc23)cc1 ; c1ccc(-c2ccc(-c3cccc(N(c4cc(-c5cc6ccccc6c6ccccc56)c5ccccc5c4)c4ccc5oc6cc7ccccc7cc6c5c4)c3)cc2)cc1 ; c1ccc(-c2ccc(-c3ccccc3N(c3ccc4oc5cc6ccccc6cc5c4c3)c3cc(-c4cc5ccccc5c5ccccc45)cc4ccccc34)cc2)cc1]

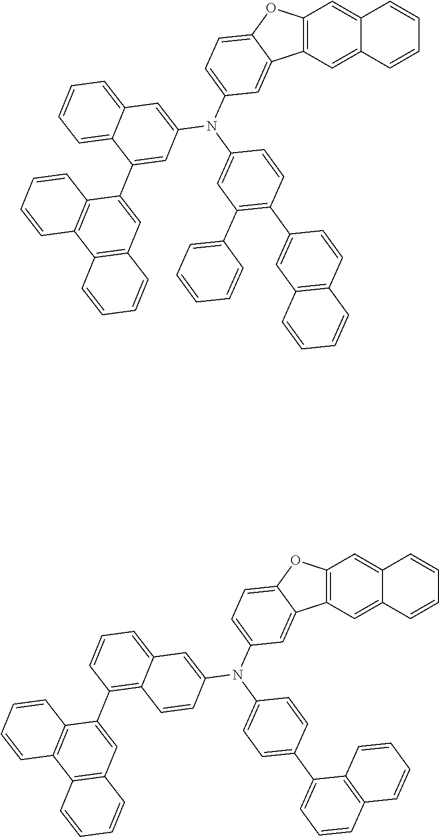 [SMILES: c1ccc(-c2cc(N(c3cc(-c4cc5ccccc5c5ccccc45)c4ccccc4c3)c3ccc4oc5cc6ccccc6cc5c4c3)ccc2-c2ccc3ccccc3c2)cc1 ; c1ccc2cc3c(cc2c1)oc1ccc(N(c2ccc(-c4cccc5ccccc45)cc2)c2ccc4c(-c5cc6ccccc6c6ccccc56)cccc4c2)cc13]

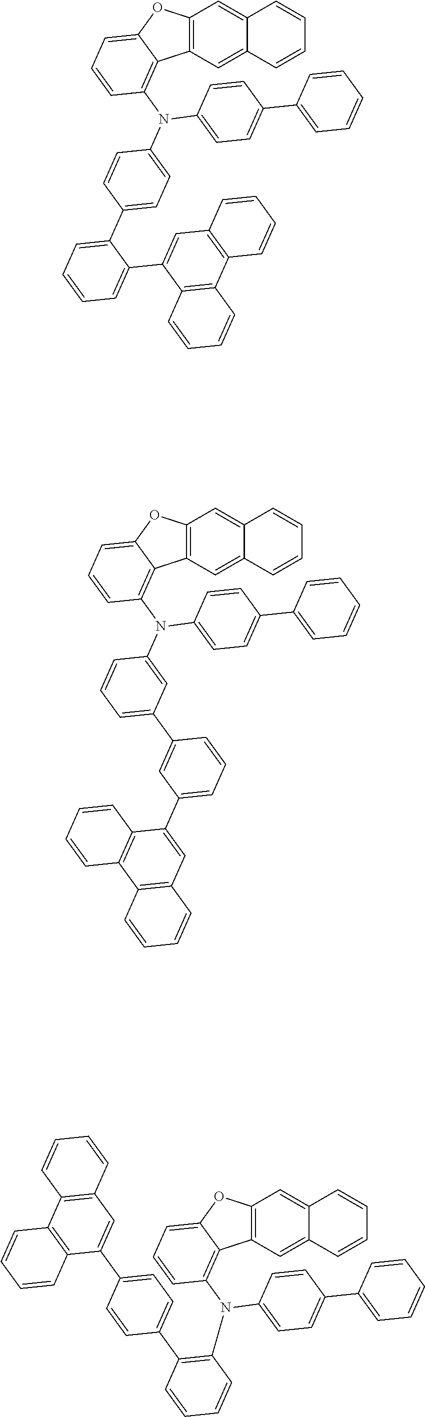 [SMILES: c1ccc(-c2ccc(N(c3ccc(-c4ccccc4-c4cc5ccccc5c5ccccc45)cc3)c3cccc4oc5cc6ccccc6cc5c34)cc2)cc1 ; c1ccc(-c2ccc(N(c3cccc(-c4cccc(-c5cc6ccccc6c6ccccc56)c4)c3)c3cccc4oc5cc6ccccc6cc5c34)cc2)cc1 ; c1ccc(-c2ccc(N(c3ccccc3-c3ccc(-c4cc5ccccc5c5ccccc45)cc3)c3cccc4oc5cc6ccccc6cc5c34)cc2)cc1]

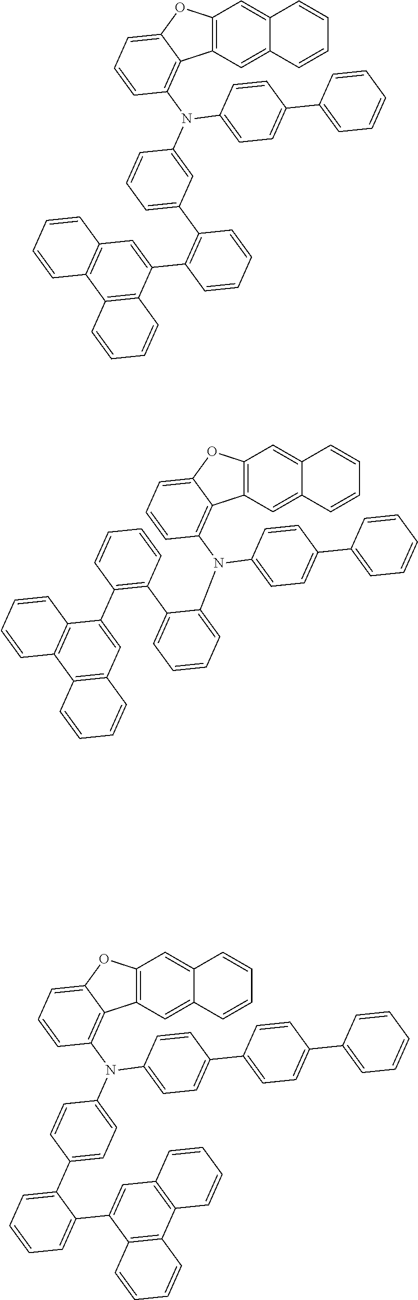 [SMILES: c1ccc(-c2ccc(N(c3cccc(-c4ccccc4-c4cc5ccccc5c5ccccc45)c3)c3cccc4oc5cc6ccccc6cc5c34)cc2)cc1 ; c1ccc(-c2ccc(N(c3ccccc3-c3ccccc3-c3cc4ccccc4c4ccccc34)c3cccc4oc5cc6ccccc6cc5c34)cc2)cc1 ; c1ccc(-c2ccc(-c3ccc(N(c4ccc(-c5ccccc5-c5cc6ccccc6c6ccccc56)cc4)c4cccc5oc6cc7ccccc7cc6c45)cc3)cc2)cc1]

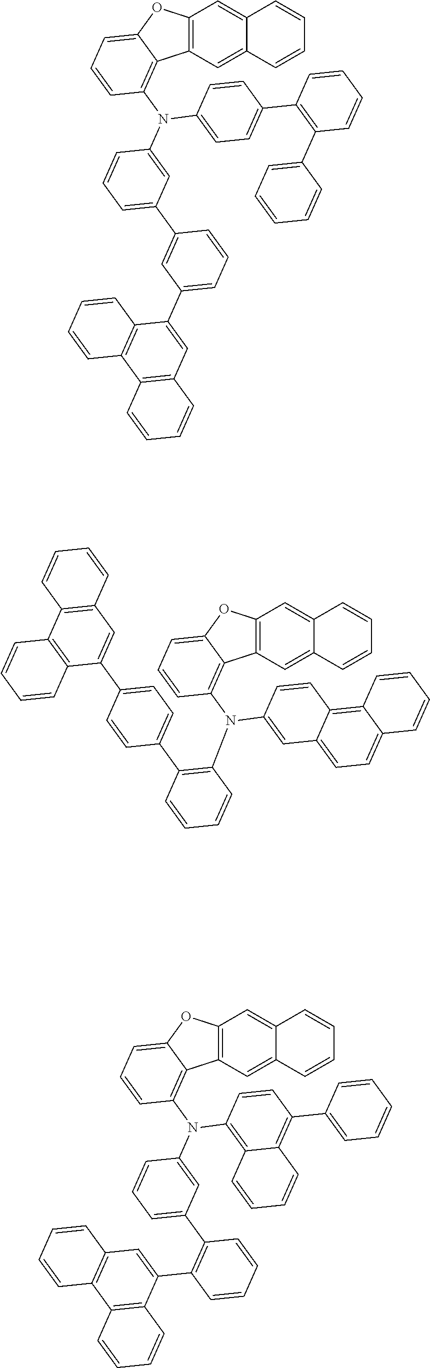 [SMILES: c1ccc(-c2ccccc2-c2ccc(N(c3cccc(-c4cccc(-c5cc6ccccc6c6ccccc56)c4)c3)c3cccc4oc5cc6ccccc6cc5c34)cc2)cc1 ; c1ccc(N(c2ccc3c(ccc4ccccc43)c2)c2cccc3oc4cc5ccccc5cc4c23)c(-c2ccc(-c3cc4ccccc4c4ccccc34)cc2)c1 ; c1ccc(-c2ccc(N(c3cccc(-c4ccccc4-c4cc5ccccc5c5ccccc45)c3)c3cccc4oc5cc6ccccc6cc5c34)c3ccccc23)cc1]

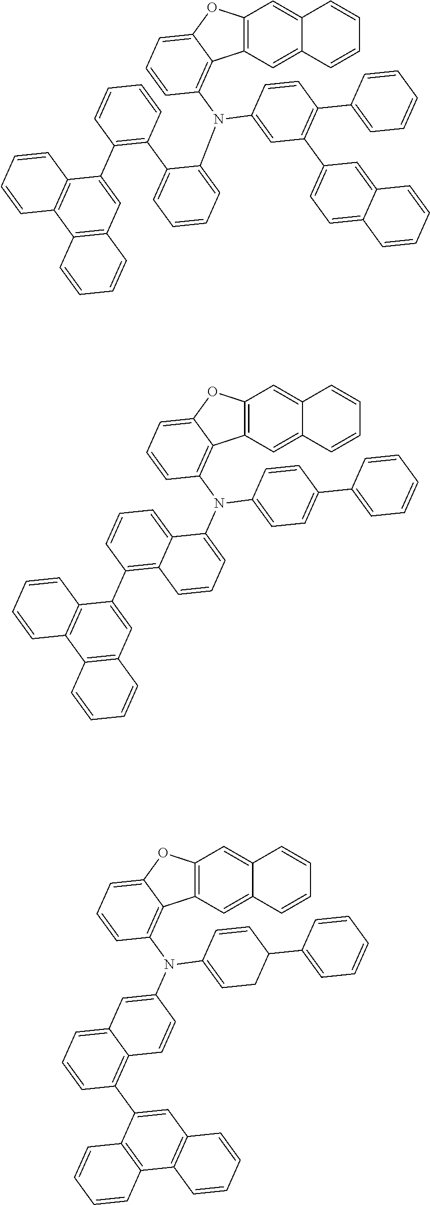 [SMILES: c1ccc(-c2ccc(N(c3ccccc3-c3ccccc3-c3cc4ccccc4c4ccccc34)c3cccc4oc5cc6ccccc6cc5c34)cc2-c2ccc3ccccc3c2)cc1 ; c1ccc(-c2ccc(N(c3cccc4c(-c5cc6ccccc6c6ccccc56)cccc34)c3cccc4oc5cc6ccccc6cc5c34)cc2)cc1 ; C1=CC(c2ccccc2)CC=C1N(c1ccc2c(-c3cc4ccccc4c4ccccc34)cccc2c1)c1cccc2oc3cc4ccccc4cc3c12]

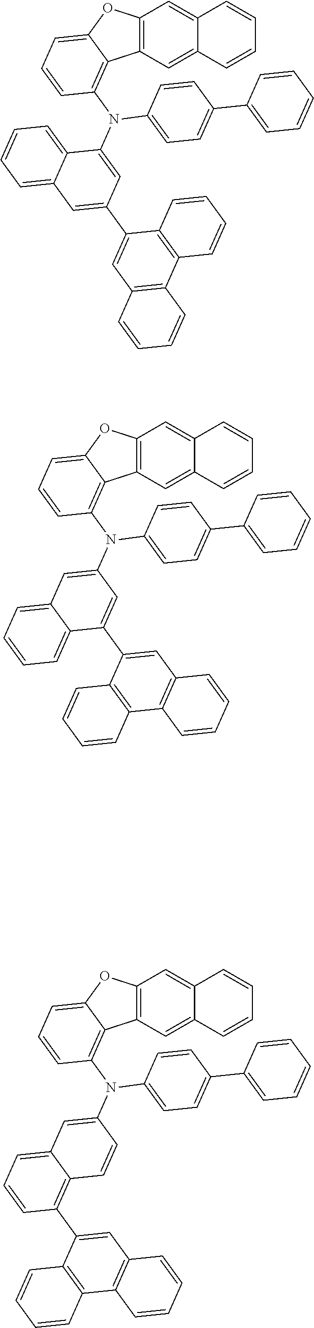 [SMILES: c1ccc(-c2ccc(N(c3cc(-c4cc5ccccc5c5ccccc45)cc4ccccc34)c3cccc4oc5cc6ccccc6cc5c34)cc2)cc1 ; c1ccc(-c2ccc(N(c3cc(-c4cc5ccccc5c5ccccc45)c4ccccc4c3)c3cccc4oc5cc6ccccc6cc5c34)cc2)cc1 ; c1ccc(-c2ccc(N(c3ccc4c(-c5cc6ccccc6c6ccccc56)cccc4c3)c3cccc4oc5cc6ccccc6cc5c34)cc2)cc1]

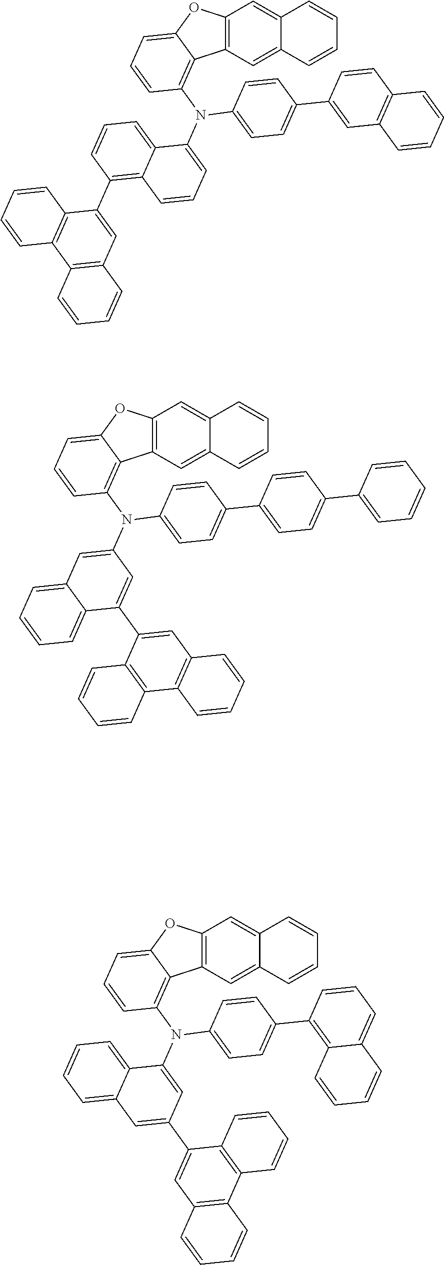 [SMILES: c1ccc2cc(-c3ccc(N(c4cccc5c(-c6cc7ccccc7c7ccccc67)cccc45)c4cccc5oc6cc7ccccc7cc6c45)cc3)ccc2c1 ; c1ccc(-c2ccc(-c3ccc(N(c4cc(-c5cc6ccccc6c6ccccc56)c5ccccc5c4)c4cccc5oc6cc7ccccc7cc6c45)cc3)cc2)cc1 ; c1ccc2cc3c(cc2c1)oc1cccc(N(c2ccc(-c4cccc5ccccc45)cc2)c2cc(-c4cc5ccccc5c5ccccc45)cc4ccccc24)c13]

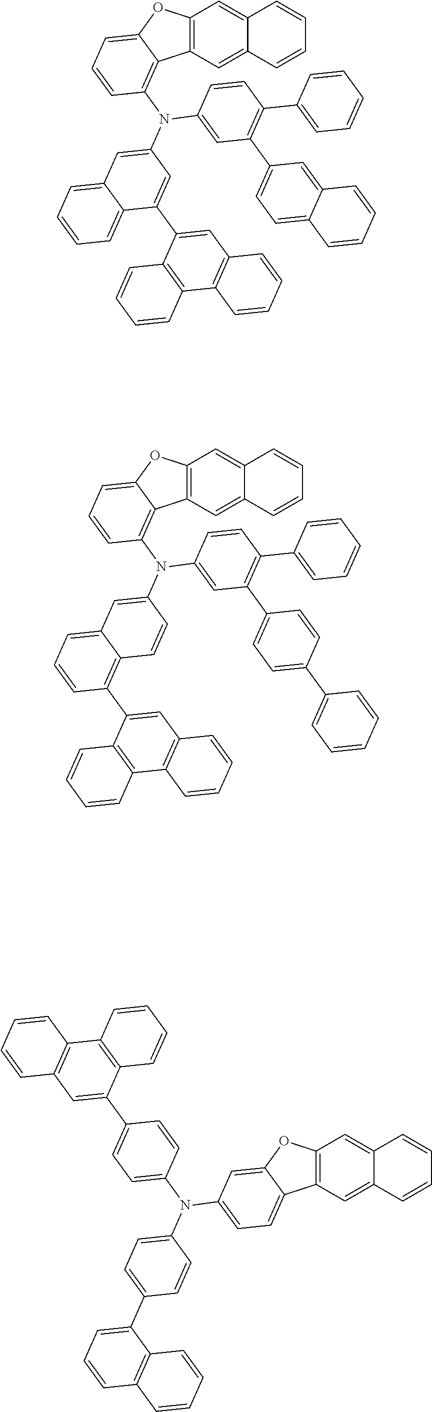 [SMILES: c1ccc(-c2ccc(N(c3cc(-c4cc5ccccc5c5ccccc45)c4ccccc4c3)c3cccc4oc5cc6ccccc6cc5c34)cc2-c2ccc3ccccc3c2)cc1 ; c1ccc(-c2ccc(-c3cc(N(c4ccc5c(-c6cc7ccccc7c7ccccc67)cccc5c4)c4cccc5oc6cc7ccccc7cc6c45)ccc3-c3ccccc3)cc2)cc1 ; c1ccc2cc3c(cc2c1)oc1cc(N(c2ccc(-c4cccc5ccccc45)cc2)c2ccc(-c4cc5ccccc5c5ccccc45)cc2)ccc13]

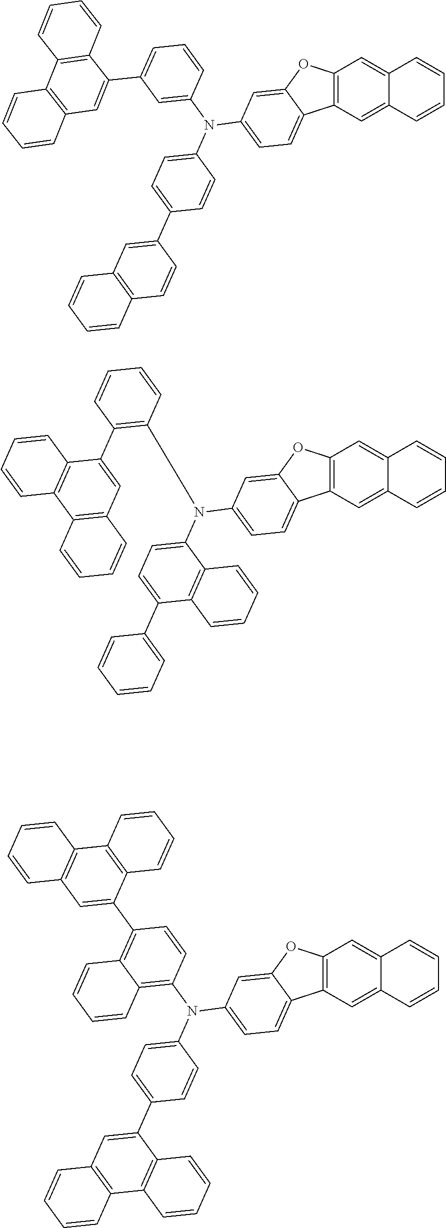

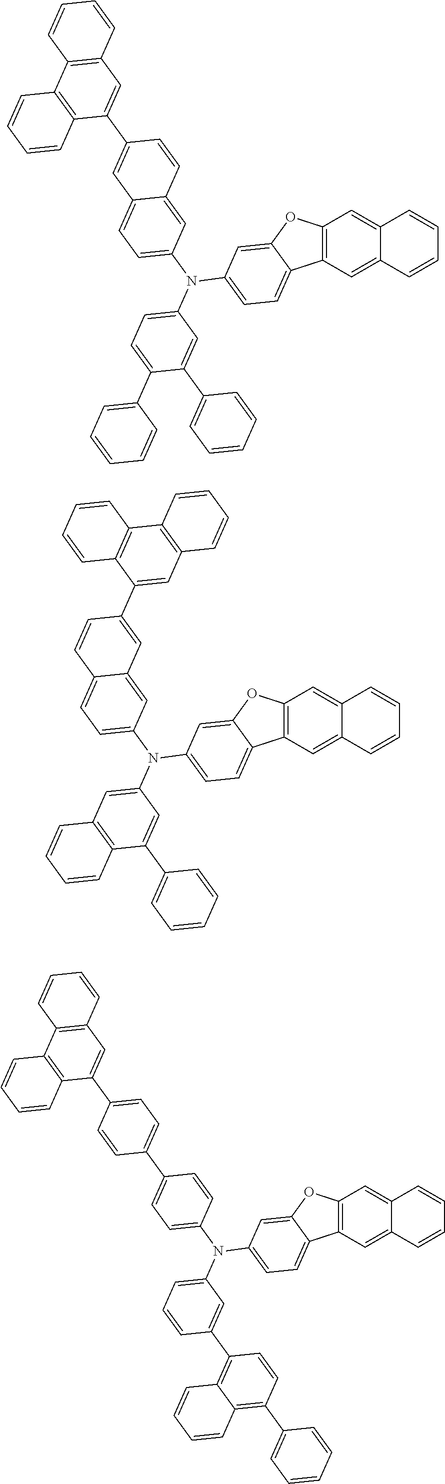

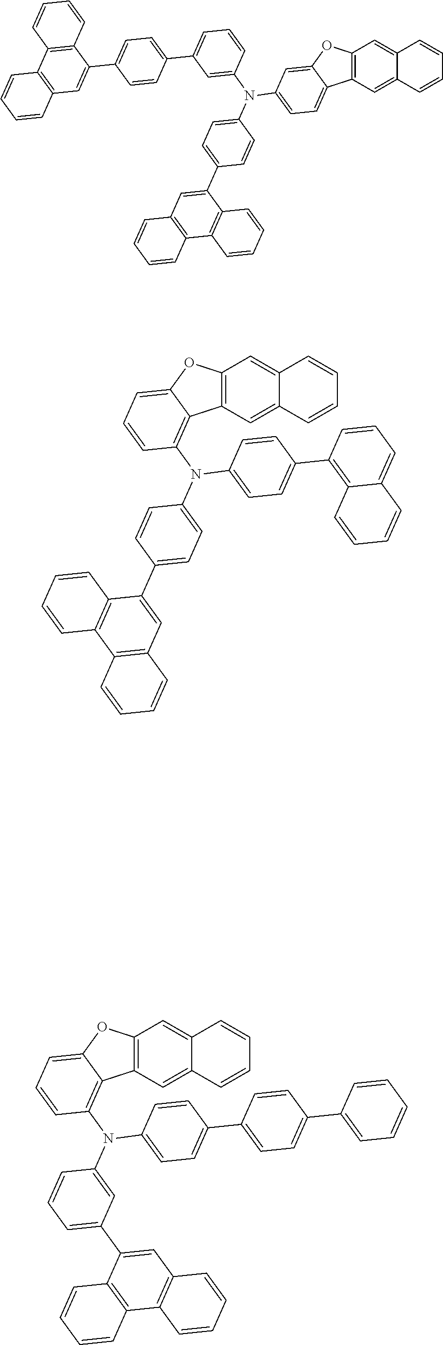 [SMILES: c1cc(-c2ccc(-c3cc4ccccc4c4ccccc34)cc2)cc(N(c2ccc(-c3cc4ccccc4c4ccccc34)cc2)c2ccc3c(c2)oc2cc4ccccc4cc23)c1 ; c1ccc2cc3c(cc2c1)oc1cccc(N(c2ccc(-c4cccc5ccccc45)cc2)c2ccc(-c4cc5ccccc5c5ccccc45)cc2)c13 ; c1ccc(-c2ccc(-c3ccc(N(c4cccc(-c5cc6ccccc6c6ccccc56)c4)c4cccc5oc6cc7ccccc7cc6c45)cc3)cc2)cc1]

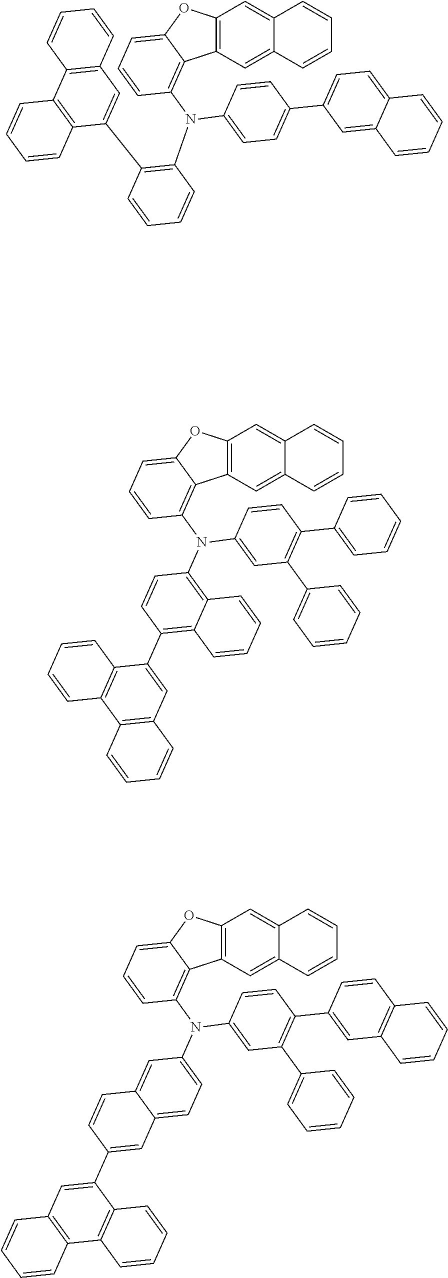 [SMILES: c1ccc(N(c2ccc(-c3ccc4ccccc4c3)cc2)c2cccc3oc4cc5ccccc5cc4c23)c(-c2cc3ccccc3c3ccccc23)c1 ; c1ccc(-c2ccc(N(c3ccc(-c4cc5ccccc5c5ccccc45)c4ccccc34)c3cccc4oc5cc6ccccc6cc5c34)cc2-c2ccccc2)cc1 ; c1ccc(-c2cc(N(c3ccc4cc(-c5cc6ccccc6c6ccccc56)ccc4c3)c3cccc4oc5cc6ccccc6cc5c34)ccc2-c2ccc3ccccc3c2)cc1]

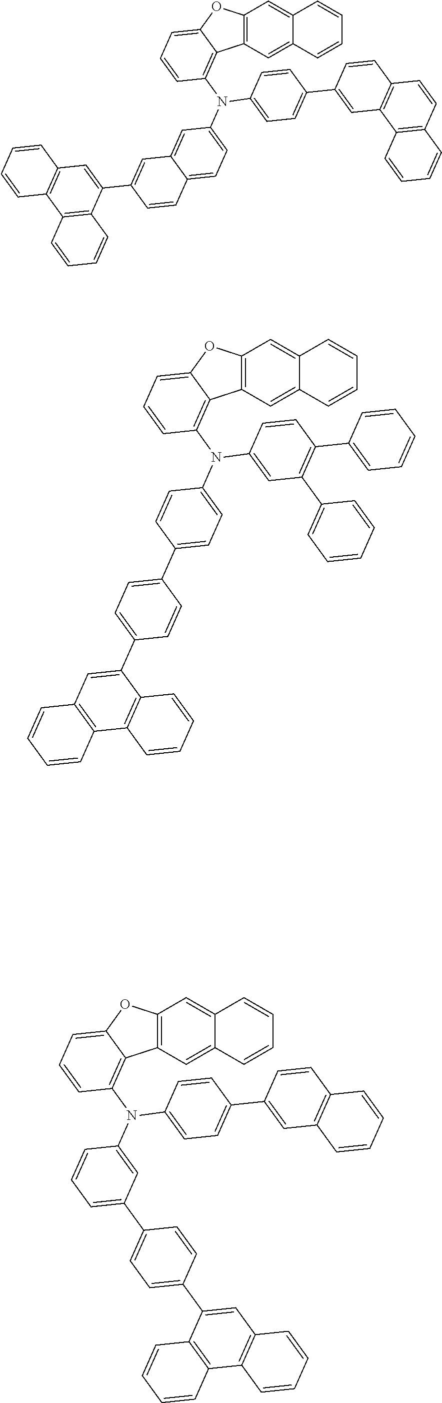 [SMILES: c1ccc2cc3c(cc2c1)oc1cccc(N(c2ccc(-c4ccc5ccc6ccccc6c5c4)cc2)c2ccc4ccc(-c5cc6ccccc6c6ccccc56)cc4c2)c13 ; c1ccc(-c2ccc(N(c3ccc(-c4ccc(-c5cc6ccccc6c6ccccc56)cc4)cc3)c3cccc4oc5cc6ccccc6cc5c34)cc2-c2ccccc2)cc1 ; c1cc(-c2ccc(-c3cc4ccccc4c4ccccc34)cc2)cc(N(c2ccc(-c3ccc4ccccc4c3)cc2)c2cccc3oc4cc5ccccc5cc4c23)c1]

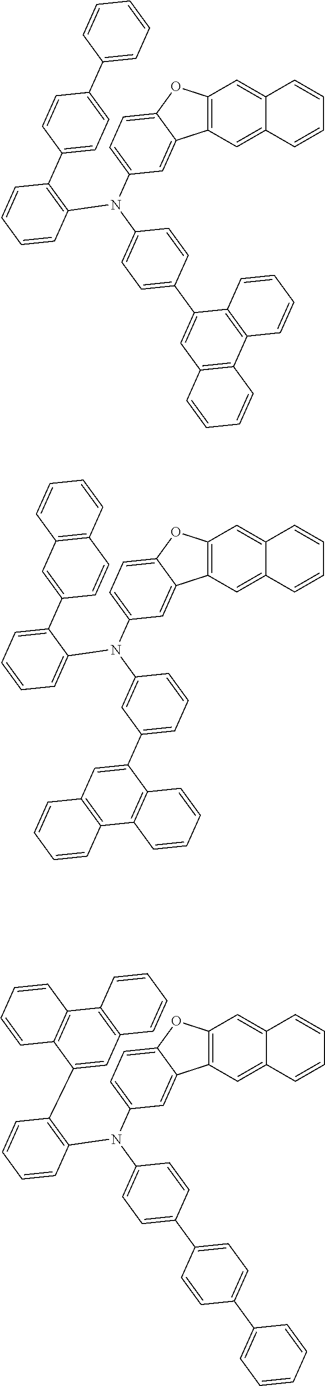 [SMILES: c1ccc(-c2ccc(-c3ccccc3N(c3ccc(-c4cc5ccccc5c5ccccc45)cc3)c3ccc4oc5cc6ccccc6cc5c4c3)cc2)cc1 ; c1cc(-c2cc3ccccc3c3ccccc23)cc(N(c2ccc3oc4cc5ccccc5cc4c3c2)c2ccccc2-c2ccc3ccccc3c2)c1 ; c1ccc(-c2ccc(-c3ccc(N(c4ccc5oc6cc7ccccc7cc6c5c4)c4ccccc4-c4cc5ccccc5c5ccccc45)cc3)cc2)cc1]

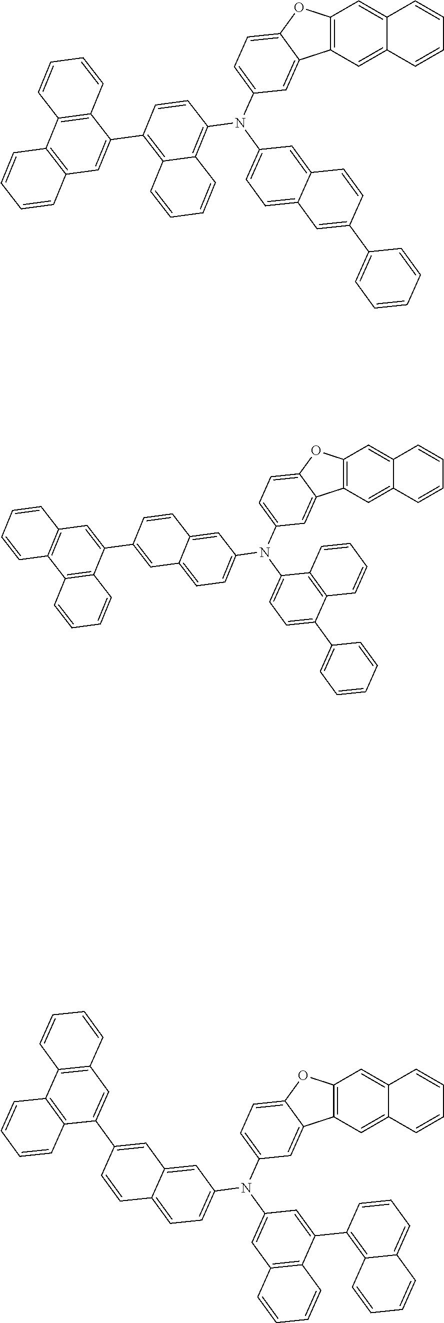 [SMILES: c1ccc(-c2ccc3cc(N(c4ccc5oc6cc7ccccc7cc6c5c4)c4ccc(-c5cc6ccccc6c6ccccc56)c5ccccc45)ccc3c2)cc1 ; c1ccc(-c2ccc(N(c3ccc4cc(-c5cc6ccccc6c6ccccc56)ccc4c3)c3ccc4oc5cc6ccccc6cc5c4c3)c3ccccc23)cc1 ; c1ccc2cc3c(cc2c1)oc1ccc(N(c2ccc4ccc(-c5cc6ccccc6c6ccccc56)cc4c2)c2cc(-c4cccc5ccccc45)c4ccccc4c2)cc13]

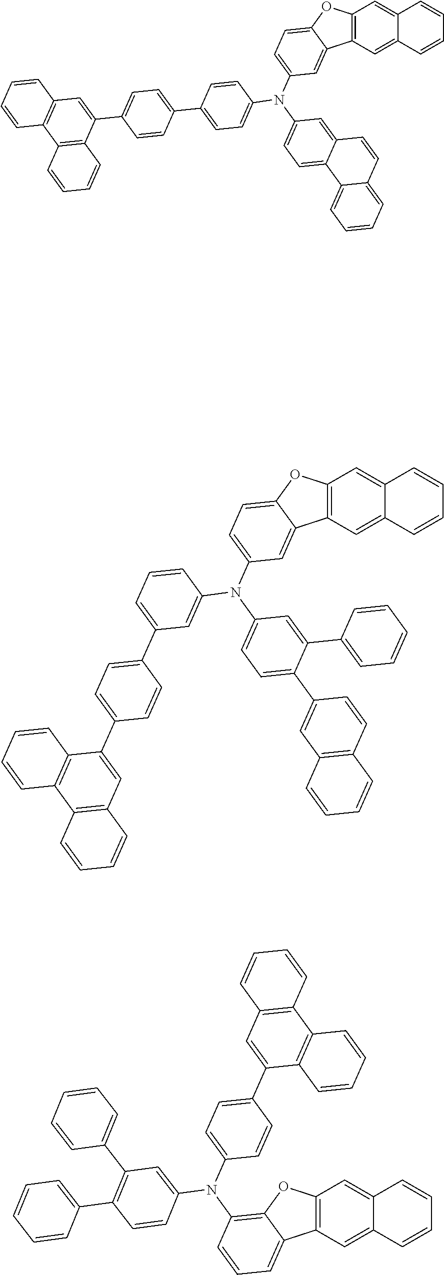 [SMILES: c1ccc2cc3c(cc2c1)oc1ccc(N(c2ccc(-c4ccc(-c5cc6ccccc6c6ccccc56)cc4)cc2)c2ccc4c(ccc5ccccc54)c2)cc13 ; c1ccc(-c2cc(N(c3cccc(-c4ccc(-c5cc6ccccc6c6ccccc56)cc4)c3)c3ccc4oc5cc6ccccc6cc5c4c3)ccc2-c2ccc3ccccc3c2)cc1 ; c1ccc(-c2ccc(N(c3ccc(-c4cc5ccccc5c5ccccc45)cc3)c3cccc4c3oc3cc5ccccc5cc34)cc2-c2ccccc2)cc1]

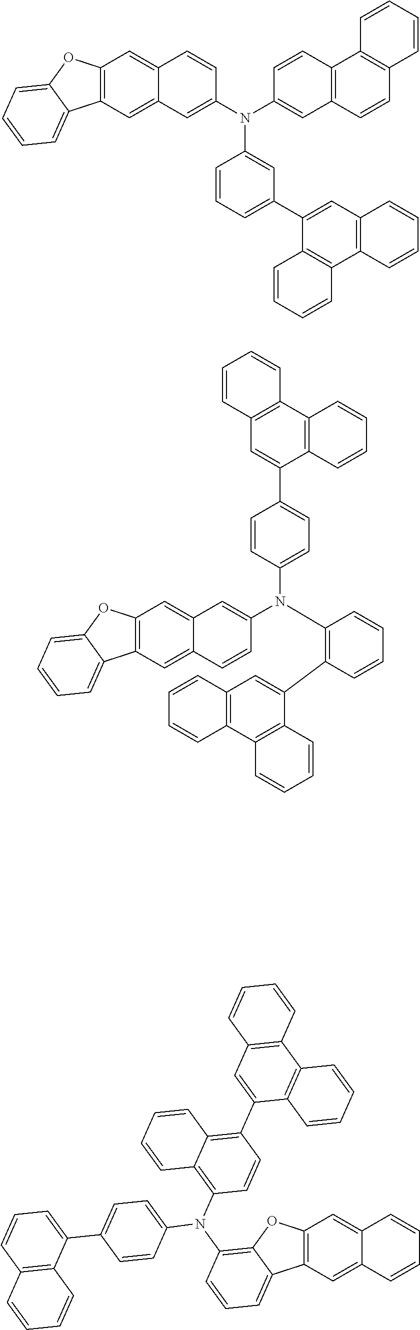 [SMILES: c1cc(-c2cc3ccccc3c3ccccc23)cc(N(c2ccc3cc4oc5ccccc5c4cc3c2)c2ccc3c(ccc4ccccc43)c2)c1 ; c1ccc(N(c2ccc(-c3cc4ccccc4c4ccccc34)cc2)c2ccc3cc4c(cc3c2)oc2ccccc24)c(-c2cc3ccccc3c3ccccc23)c1 ; c1ccc2cc3c(cc2c1)oc1c(N(c2ccc(-c4cccc5ccccc45)cc2)c2ccc(-c4cc5ccccc5c5ccccc45)c4ccccc24)cccc13]

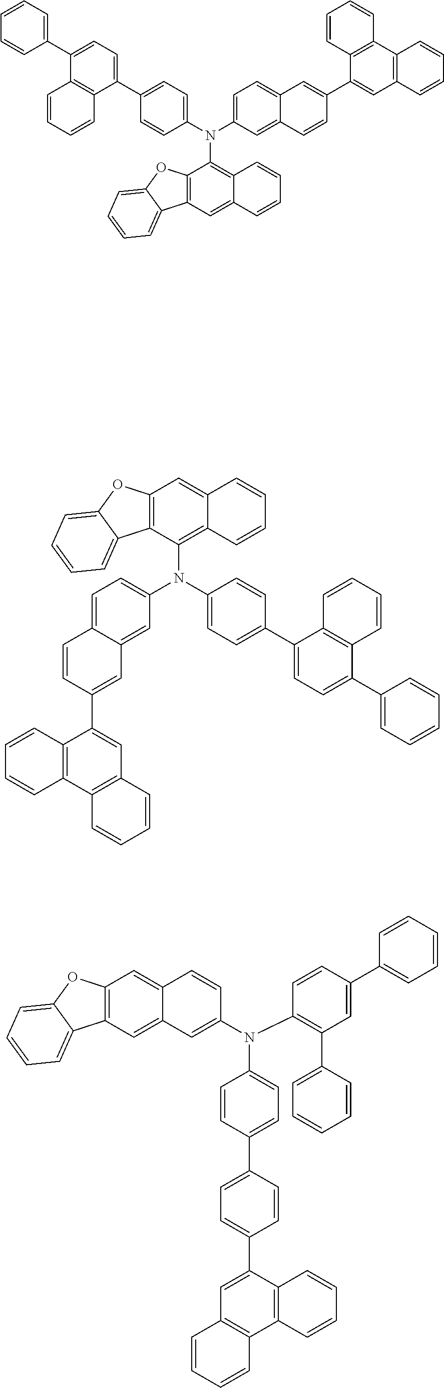 [SMILES: c1ccc(-c2ccc(-c3ccc(N(c4ccc5cc(-c6cc7ccccc7c7ccccc67)ccc5c4)c4c5ccccc5cc5c4oc4ccccc45)cc3)c3ccccc23)cc1 ; c1ccc(-c2ccc(-c3ccc(N(c4ccc5ccc(-c6cc7ccccc7c7ccccc67)cc5c4)c4c5ccccc5cc5oc6ccccc6c45)cc3)c3ccccc23)cc1 ; c1ccc(-c2ccc(N(c3ccc(-c4ccc(-c5cc6ccccc6c6ccccc56)cc4)cc3)c3ccc4cc5oc6ccccc6c5cc4c3)c(-c3ccccc3)c2)cc1]

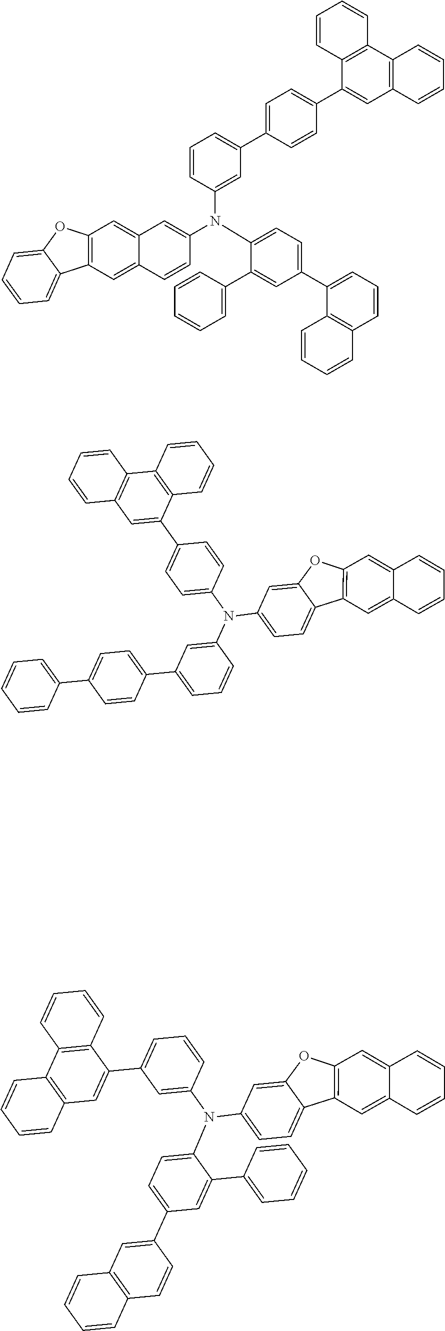 [SMILES: c1ccc(-c2cc(-c3cccc4ccccc34)ccc2N(c2cccc(-c3ccc(-c4cc5ccccc5c5ccccc45)cc3)c2)c2ccc3cc4c(cc3c2)oc2ccccc24)cc1 ; c1ccc(-c2ccc(-c3cccc(N(c4ccc(-c5cc6ccccc6c6ccccc56)cc4)c4ccc5c(c4)oc4cc6ccccc6cc45)c3)cc2)cc1 ; c1ccc(-c2cc(-c3ccc4ccccc4c3)ccc2N(c2cccc(-c3cc4ccccc4c4ccccc34)c2)c2ccc3c(c2)oc2cc4ccccc4cc23)cc1]

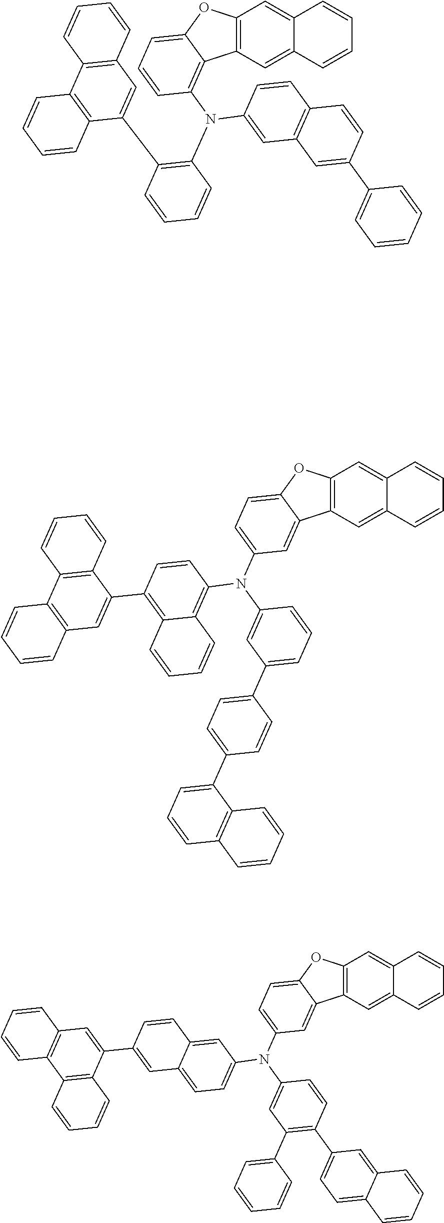 [SMILES: c1ccc(-c2ccc3ccc(N(c4ccccc4-c4cc5ccccc5c5ccccc45)c4cccc5oc6cc7ccccc7cc6c45)cc3c2)cc1 ; c1cc(-c2ccc(-c3cccc4ccccc34)cc2)cc(N(c2ccc3oc4cc5ccccc5cc4c3c2)c2ccc(-c3cc4ccccc4c4ccccc34)c3ccccc23)c1 ; c1ccc(-c2cc(N(c3ccc4cc(-c5cc6ccccc6c6ccccc56)ccc4c3)c3ccc4oc5cc6ccccc6cc5c4c3)ccc2-c2ccc3ccccc3c2)cc1]

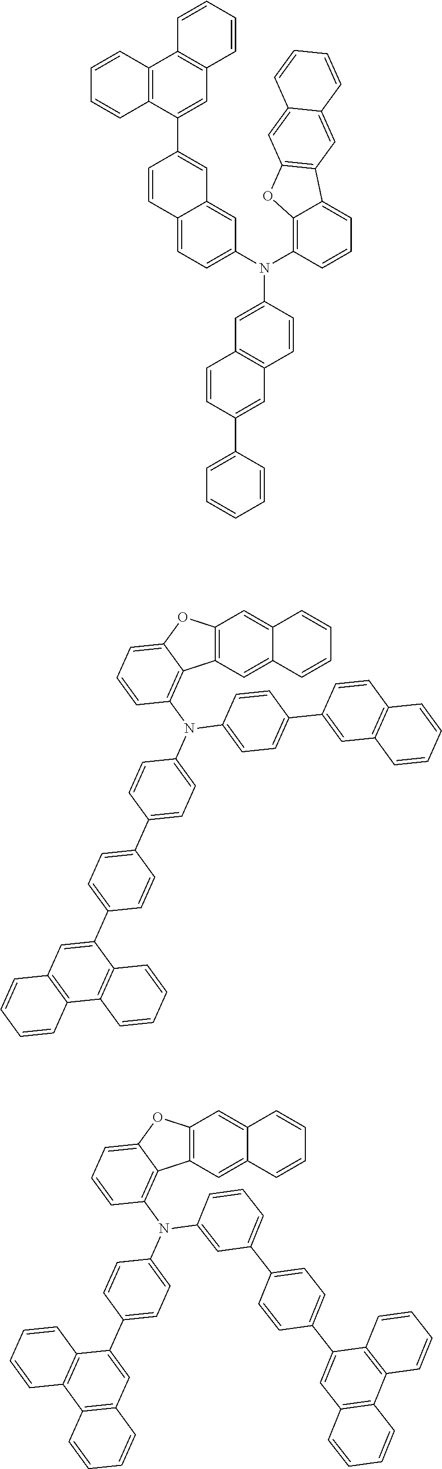 [SMILES: c1ccc(-c2ccc3cc(N(c4ccc5ccc(-c6cc7ccccc7c7ccccc67)cc5c4)c4cccc5c4oc4cc6ccccc6cc45)ccc3c2)cc1 ; c1ccc2cc(-c3ccc(N(c4ccc(-c5ccc(-c6cc7ccccc7c7ccccc67)cc5)cc4)c4cccc5oc6cc7ccccc7cc6c45)cc3)ccc2c1 ; c1cc(-c2ccc(-c3cc4ccccc4c4ccccc34)cc2)cc(N(c2ccc(-c3cc4ccccc4c4ccccc34)cc2)c2cccc3oc4cc5ccccc5cc4c23)c1]

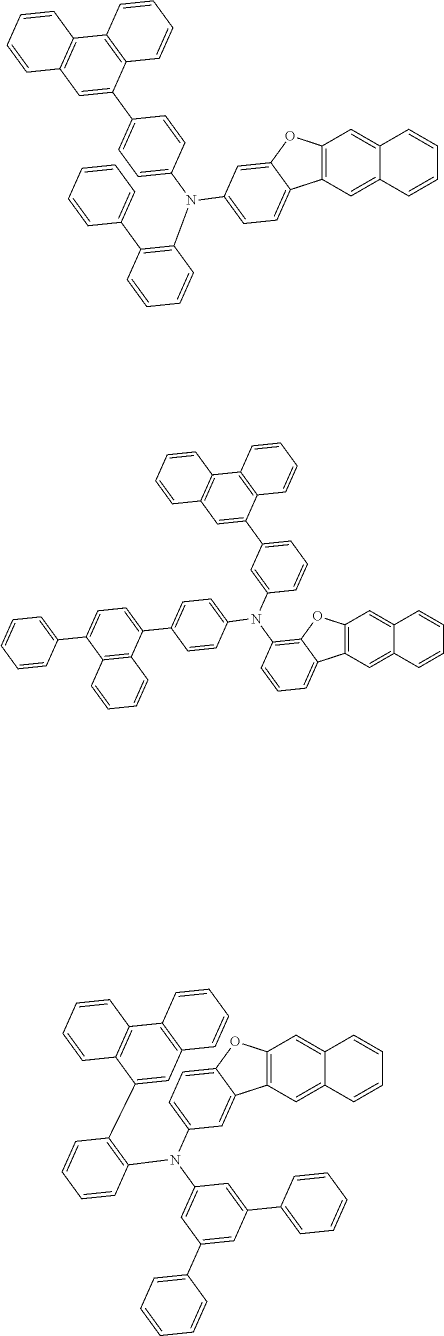 [SMILES: c1ccc(-c2ccccc2N(c2ccc(-c3cc4ccccc4c4ccccc34)cc2)c2ccc3c(c2)oc2cc4ccccc4cc23)cc1 ; c1ccc(-c2ccc(-c3ccc(N(c4cccc(-c5cc6ccccc6c6ccccc56)c4)c4cccc5c4oc4cc6ccccc6cc45)cc3)c3ccccc23)cc1 ; c1ccc(-c2cc(-c3ccccc3)cc(N(c3ccc4oc5cc6ccccc6cc5c4c3)c3ccccc3-c3cc4ccccc4c4ccccc34)c2)cc1]

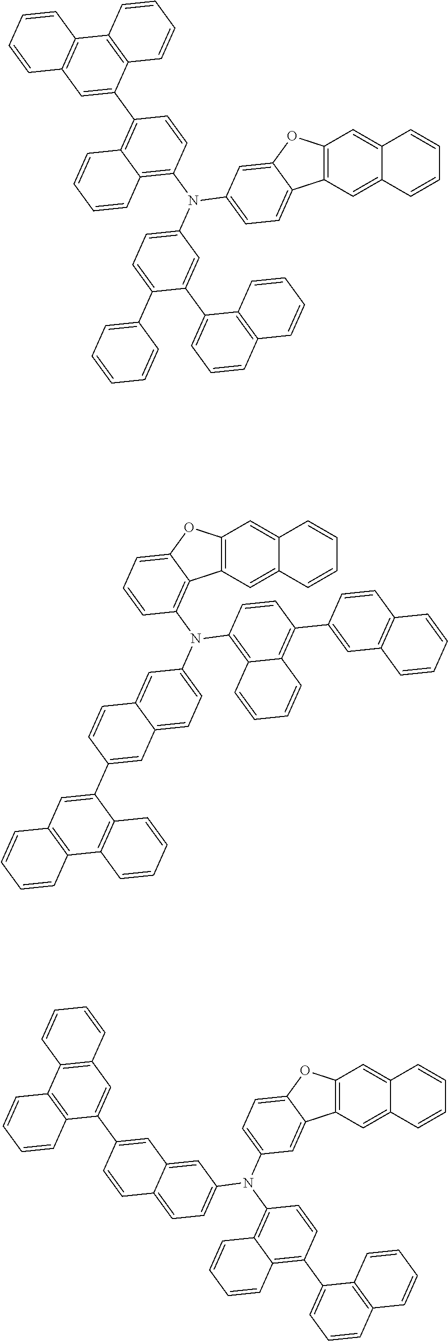 [SMILES: c1ccc(-c2ccc(N(c3ccc4c(c3)oc3cc5ccccc5cc34)c3ccc(-c4cc5ccccc5c5ccccc45)c4ccccc34)cc2-c2cccc3ccccc23)cc1 ; c1ccc2cc(-c3ccc(N(c4ccc5cc(-c6cc7ccccc7c7ccccc67)ccc5c4)c4cccc5oc6cc7ccccc7cc6c45)c4ccccc34)ccc2c1 ; c1ccc2cc3c(cc2c1)oc1ccc(N(c2ccc4ccc(-c5cc6ccccc6c6ccccc56)cc4c2)c2ccc(-c4cccc5ccccc45)c4ccccc24)cc13]

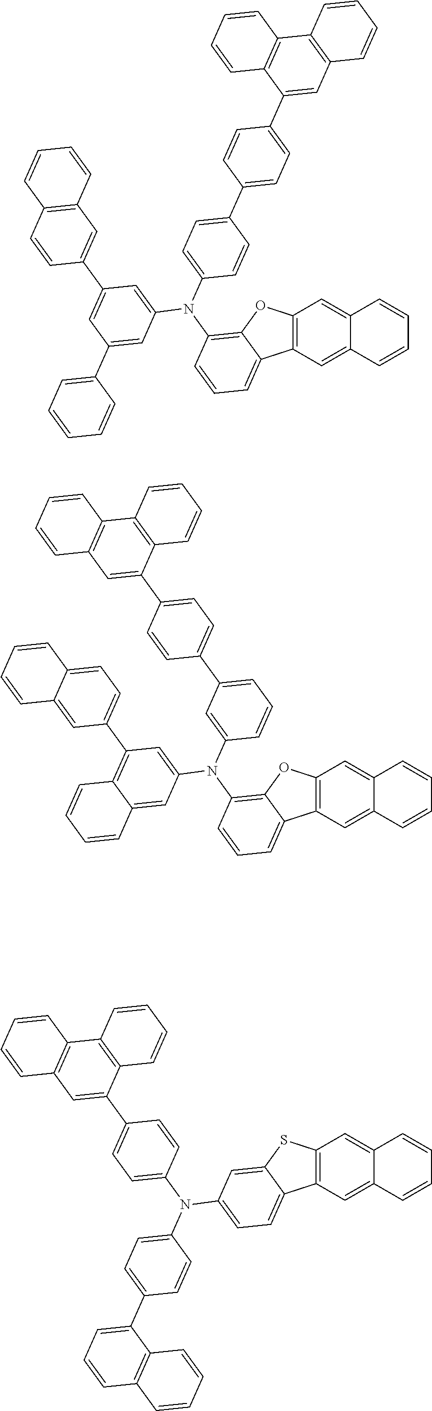 [SMILES: c1ccc(-c2cc(-c3ccc4ccccc4c3)cc(N(c3ccc(-c4ccc(-c5cc6ccccc6c6ccccc56)cc4)cc3)c3cccc4c3oc3cc5ccccc5cc34)c2)cc1 ; c1cc(-c2ccc(-c3cc4ccccc4c4ccccc34)cc2)cc(N(c2cc(-c3ccc4ccccc4c3)c3ccccc3c2)c2cccc3c2oc2cc4ccccc4cc23)c1 ; c1ccc2cc3c(cc2c1)sc1cc(N(c2ccc(-c4cccc5ccccc45)cc2)c2ccc(-c4cc5ccccc5c5ccccc45)cc2)ccc13]

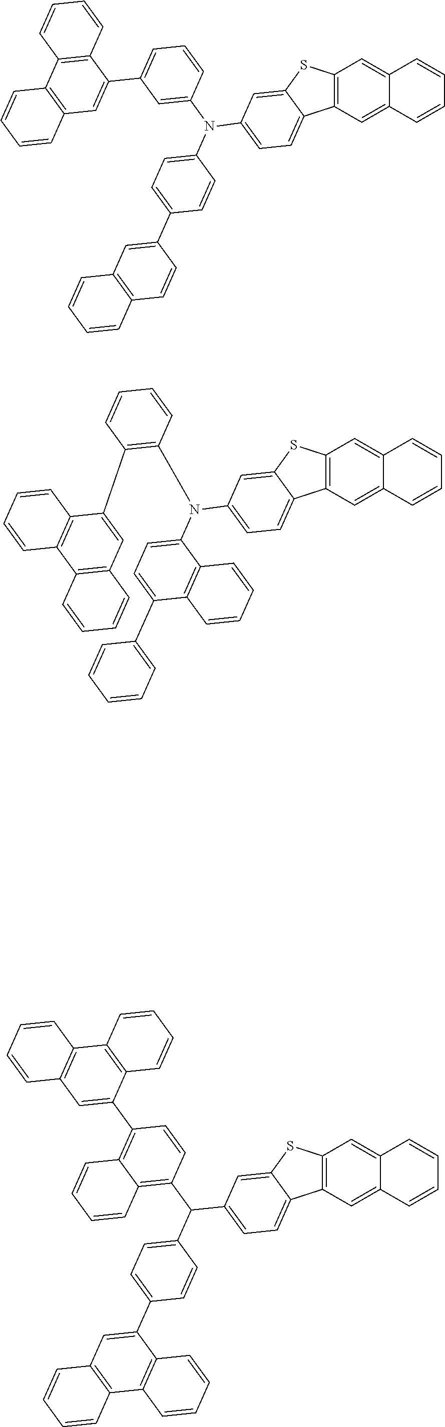 [SMILES: c1cc(-c2cc3ccccc3c3ccccc23)cc(N(c2ccc(-c3ccc4ccccc4c3)cc2)c2ccc3c(c2)sc2cc4ccccc4cc23)c1 ; c1ccc(-c2ccc(N(c3ccc4c(c3)sc3cc5ccccc5cc34)c3ccccc3-c3cc4ccccc4c4ccccc34)c3ccccc23)cc1 ; c1ccc2cc3c(cc2c1)sc1cc(C(c2ccc(-c4cc5ccccc5c5ccccc45)cc2)c2ccc(-c4cc5ccccc5c5ccccc45)c4ccccc24)ccc13]

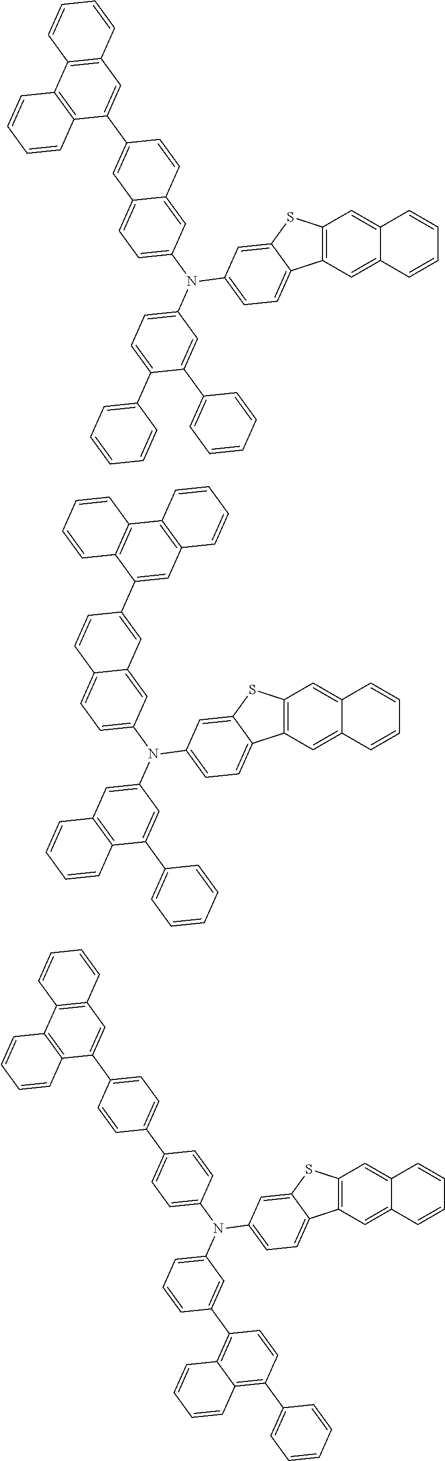

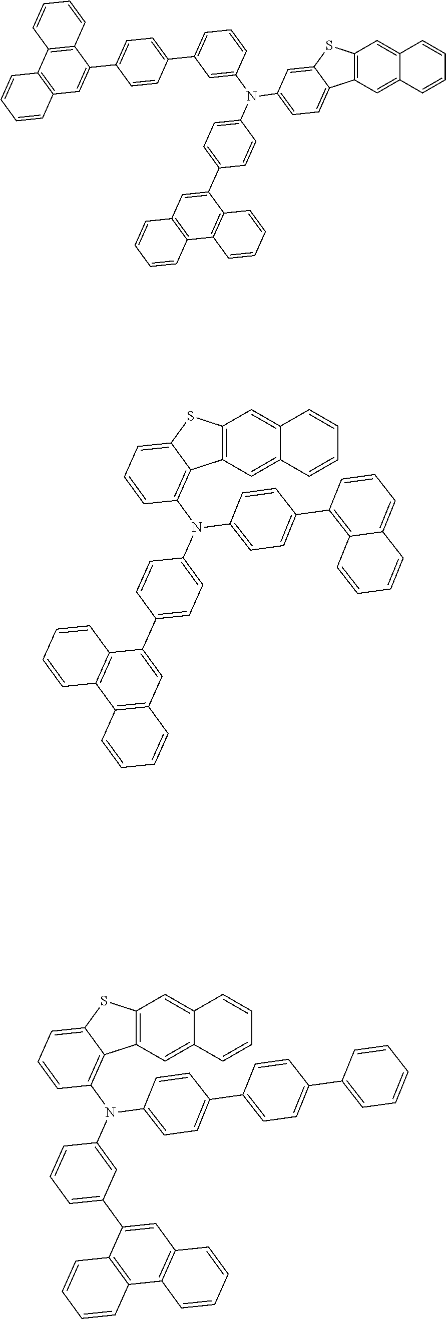 [SMILES: c1cc(-c2ccc(-c3cc4ccccc4c4ccccc34)cc2)cc(N(c2ccc(-c3cc4ccccc4c4ccccc34)cc2)c2ccc3c(c2)sc2cc4ccccc4cc23)c1 ; c1ccc2cc3c(cc2c1)sc1cccc(N(c2ccc(-c4cccc5ccccc45)cc2)c2ccc(-c4cc5ccccc5c5ccccc45)cc2)c13 ; c1ccc(-c2ccc(-c3ccc(N(c4cccc(-c5cc6ccccc6c6ccccc56)c4)c4cccc5sc6cc7ccccc7cc6c45)cc3)cc2)cc1]

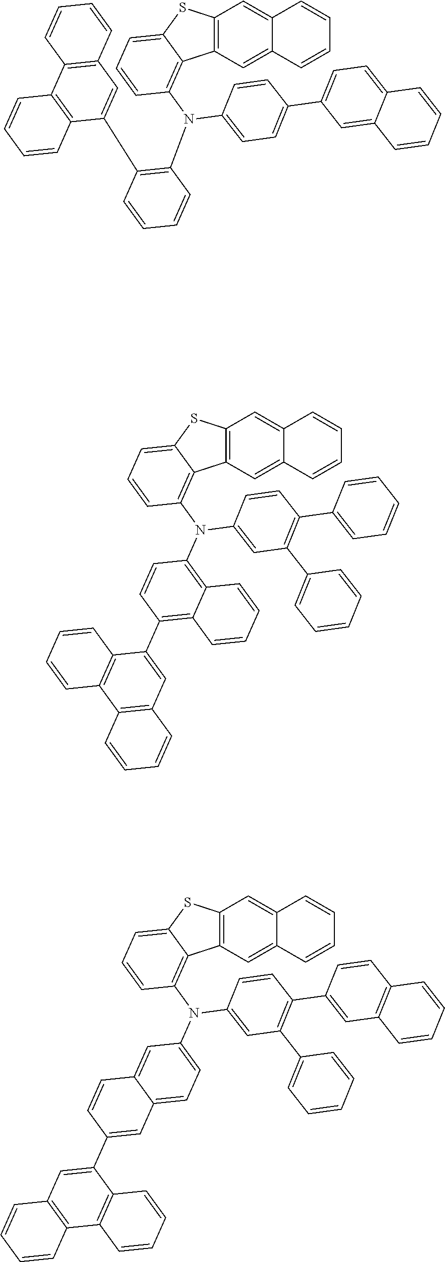 [SMILES: c1ccc(N(c2ccc(-c3ccc4ccccc4c3)cc2)c2cccc3sc4cc5ccccc5cc4c23)c(-c2cc3ccccc3c3ccccc23)c1 ; c1ccc(-c2ccc(N(c3ccc(-c4cc5ccccc5c5ccccc45)c4ccccc34)c3cccc4sc5cc6ccccc6cc5c34)cc2-c2ccccc2)cc1 ; c1ccc(-c2cc(N(c3ccc4cc(-c5cc6ccccc6c6ccccc56)ccc4c3)c3cccc4sc5cc6ccccc6cc5c34)ccc2-c2ccc3ccccc3c2)cc1]

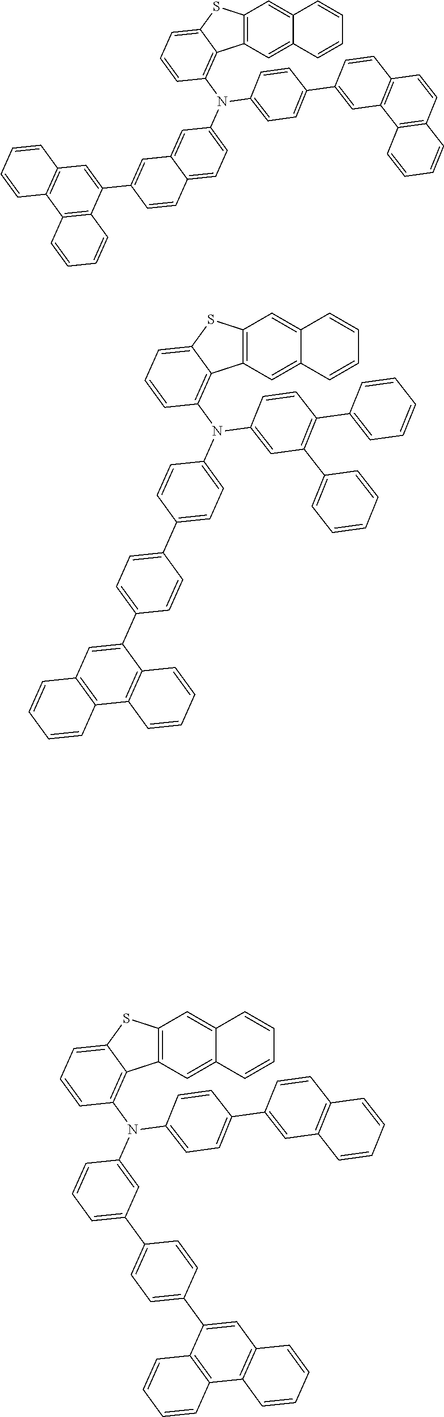 [SMILES: c1ccc2cc3c(cc2c1)sc1cccc(N(c2ccc(-c4ccc5ccc6ccccc6c5c4)cc2)c2ccc4ccc(-c5cc6ccccc6c6ccccc56)cc4c2)c13 ; c1ccc(-c2ccc(N(c3ccc(-c4ccc(-c5cc6ccccc6c6ccccc56)cc4)cc3)c3cccc4sc5cc6ccccc6cc5c34)cc2-c2ccccc2)cc1 ; c1cc(-c2ccc(-c3cc4ccccc4c4ccccc34)cc2)cc(N(c2ccc(-c3ccc4ccccc4c3)cc2)c2cccc3sc4cc5ccccc5cc4c23)c1]

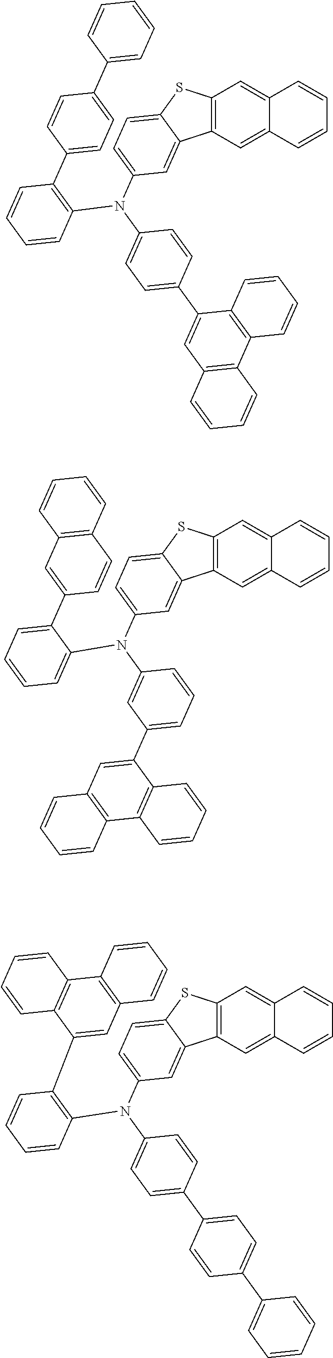 [SMILES: c1ccc(-c2ccc(-c3ccccc3N(c3ccc(-c4cc5ccccc5c5ccccc45)cc3)c3ccc4sc5cc6ccccc6cc5c4c3)cc2)cc1 ; c1cc(-c2cc3ccccc3c3ccccc23)cc(N(c2ccc3sc4cc5ccccc5cc4c3c2)c2ccccc2-c2ccc3ccccc3c2)c1 ; c1ccc(-c2ccc(-c3ccc(N(c4ccc5sc6cc7ccccc7cc6c5c4)c4ccccc4-c4cc5ccccc5c5ccccc45)cc3)cc2)cc1]

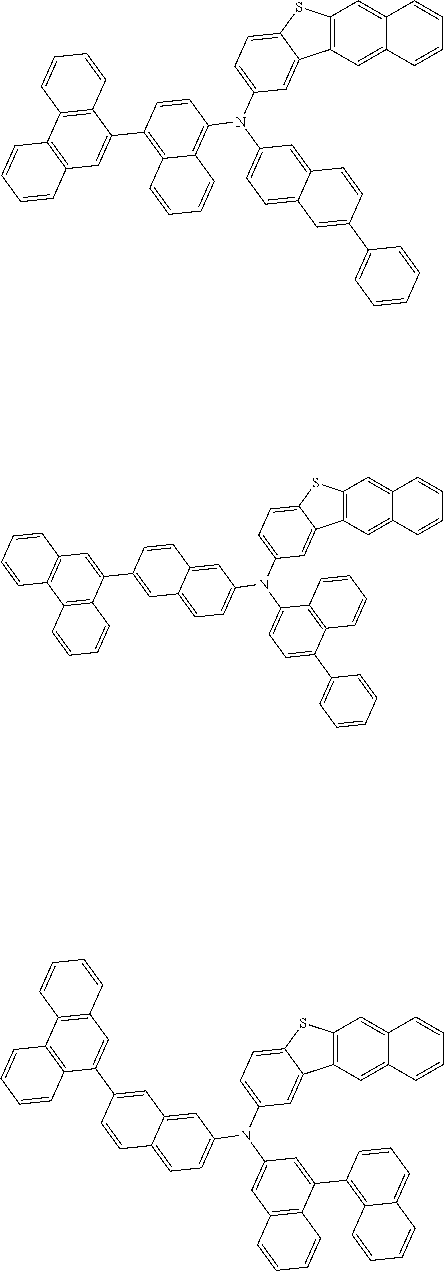 [SMILES: c1ccc(-c2ccc3cc(N(c4ccc5sc6cc7ccccc7cc6c5c4)c4ccc(-c5cc6ccccc6c6ccccc56)c5ccccc45)ccc3c2)cc1 ; c1ccc(-c2ccc(N(c3ccc4cc(-c5cc6ccccc6c6ccccc56)ccc4c3)c3ccc4sc5cc6ccccc6cc5c4c3)c3ccccc23)cc1 ; c1ccc2cc3c(cc2c1)sc1ccc(N(c2ccc4ccc(-c5cc6ccccc6c6ccccc56)cc4c2)c2cc(-c4cccc5ccccc45)c4ccccc4c2)cc13]

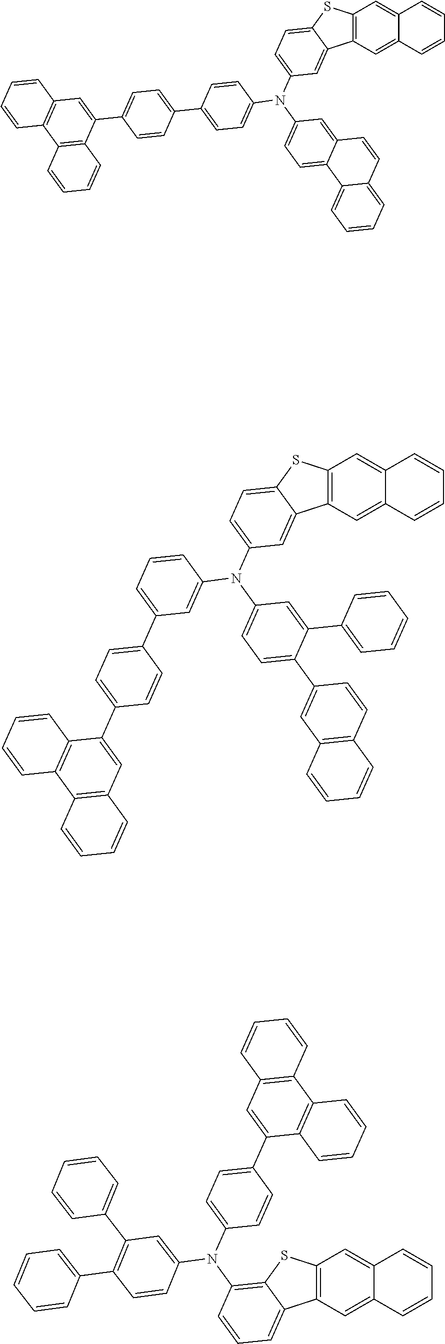 [SMILES: c1ccc2cc3c(cc2c1)sc1ccc(N(c2ccc(-c4ccc(-c5cc6ccccc6c6ccccc56)cc4)cc2)c2ccc4c(ccc5ccccc54)c2)cc13 ; c1ccc(-c2cc(N(c3cccc(-c4ccc(-c5cc6ccccc6c6ccccc56)cc4)c3)c3ccc4sc5cc6ccccc6cc5c4c3)ccc2-c2ccc3ccccc3c2)cc1 ; c1ccc(-c2ccc(N(c3ccc(-c4cc5ccccc5c5ccccc45)cc3)c3cccc4c3sc3cc5ccccc5cc34)cc2-c2ccccc2)cc1]

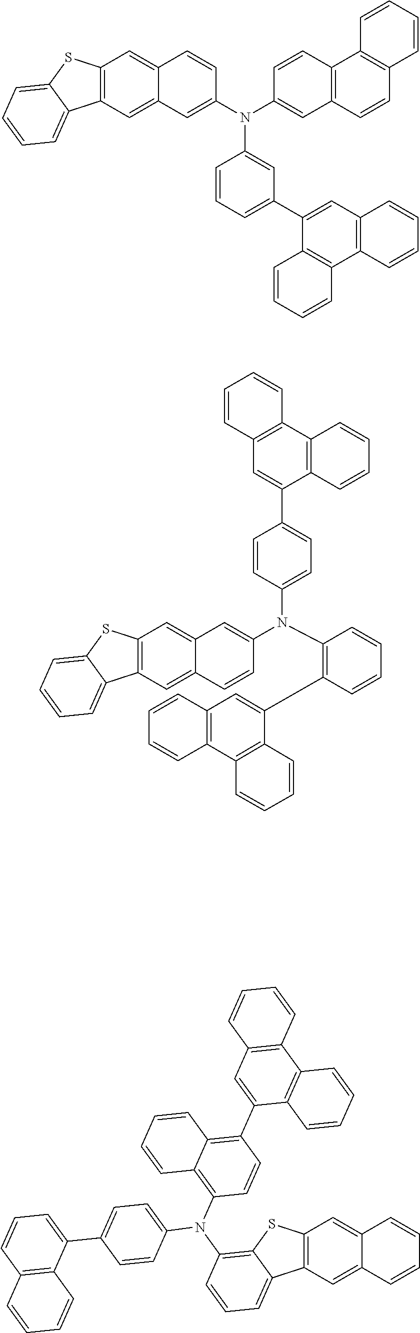 [SMILES: c1cc(-c2cc3ccccc3c3ccccc23)cc(N(c2ccc3cc4sc5ccccc5c4cc3c2)c2ccc3c(ccc4ccccc43)c2)c1 ; c1ccc(N(c2ccc(-c3cc4ccccc4c4ccccc34)cc2)c2ccc3cc4c(cc3c2)sc2ccccc24)c(-c2cc3ccccc3c3ccccc23)c1 ; c1ccc2cc3c(cc2c1)sc1c(N(c2ccc(-c4cccc5ccccc45)cc2)c2ccc(-c4cc5ccccc5c5ccccc45)c4ccccc24)cccc13]

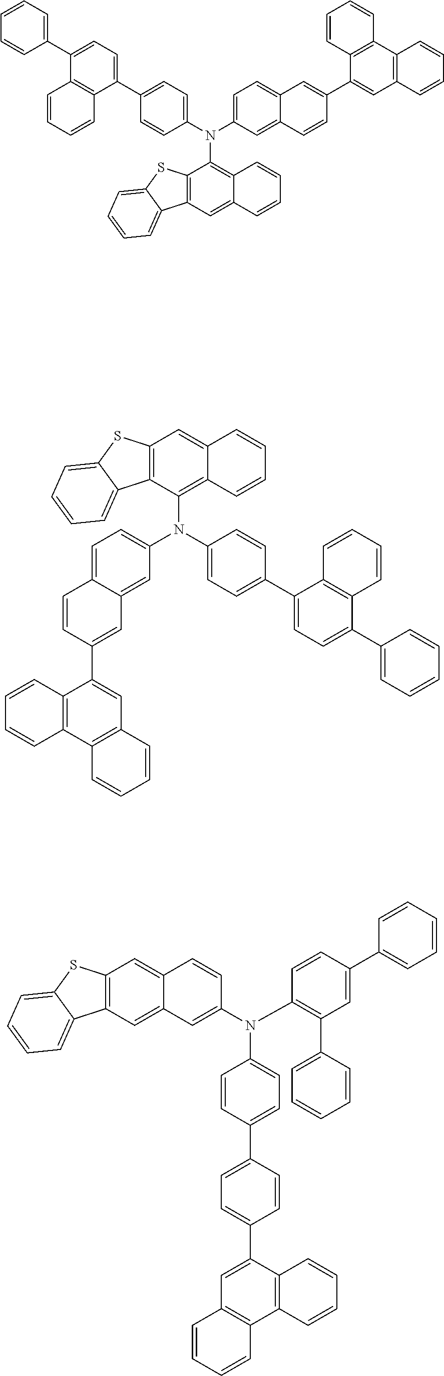 [SMILES: c1ccc(-c2ccc(-c3ccc(N(c4ccc5cc(-c6cc7ccccc7c7ccccc67)ccc5c4)c4c5ccccc5cc5c4sc4ccccc45)cc3)c3ccccc23)cc1 ; c1ccc(-c2ccc(-c3ccc(N(c4ccc5ccc(-c6cc7ccccc7c7ccccc67)cc5c4)c4c5ccccc5cc5sc6ccccc6c45)cc3)c3ccccc23)cc1 ; c1ccc(-c2ccc(N(c3ccc(-c4ccc(-c5cc6ccccc6c6ccccc56)cc4)cc3)c3ccc4cc5sc6ccccc6c5cc4c3)c(-c3ccccc3)c2)cc1]

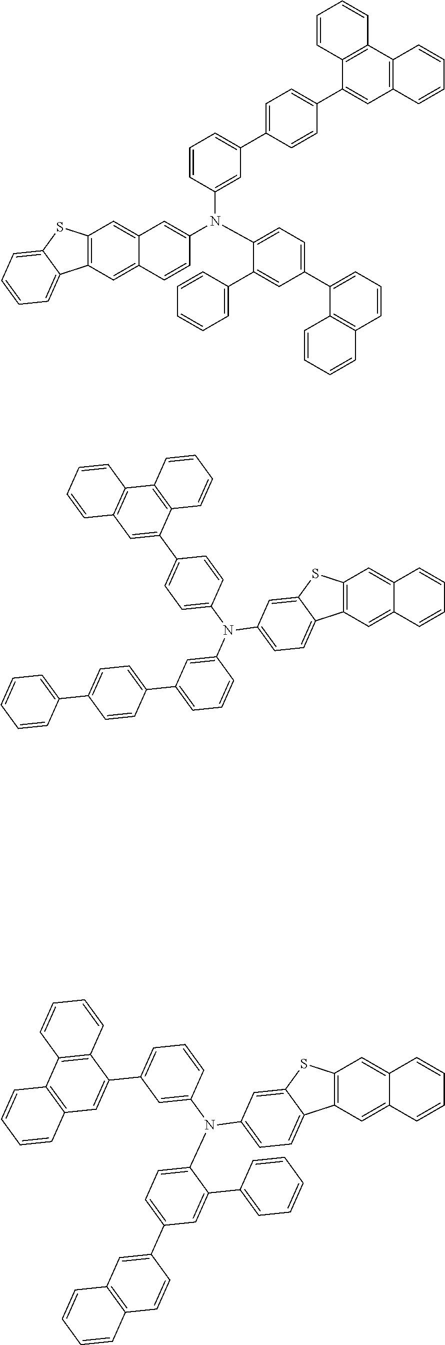 [SMILES: c1ccc(-c2cc(-c3cccc4ccccc34)ccc2N(c2cccc(-c3ccc(-c4cc5ccccc5c5ccccc45)cc3)c2)c2ccc3cc4c(cc3c2)sc2ccccc24)cc1 ; c1ccc(-c2ccc(-c3cccc(N(c4ccc(-c5cc6ccccc6c6ccccc56)cc4)c4ccc5c(c4)sc4cc6ccccc6cc45)c3)cc2)cc1 ; c1ccc(-c2cc(-c3ccc4ccccc4c3)ccc2N(c2cccc(-c3cc4ccccc4c4ccccc34)c2)c2ccc3c(c2)sc2cc4ccccc4cc23)cc1]

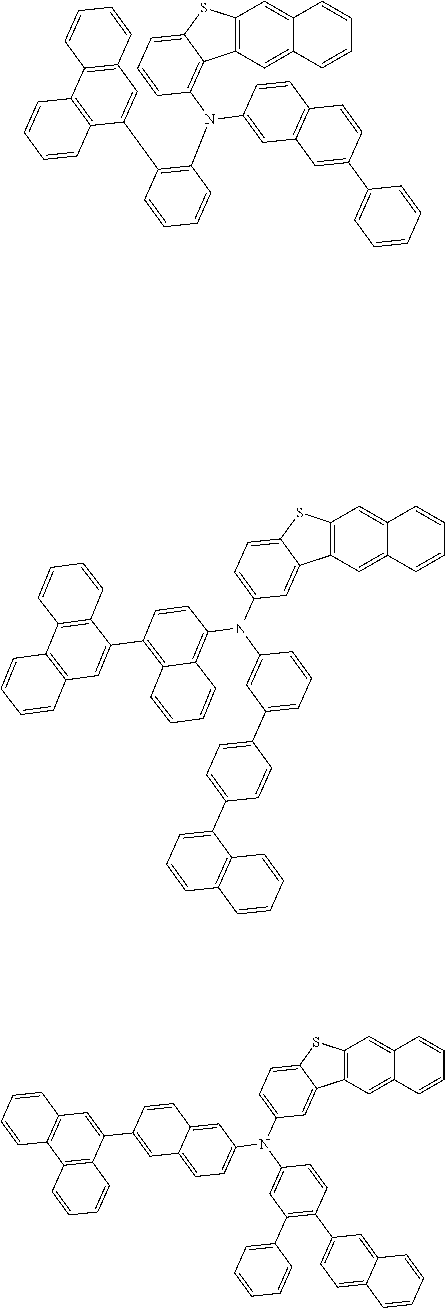 [SMILES: c1ccc(-c2ccc3ccc(N(c4ccccc4-c4cc5ccccc5c5ccccc45)c4cccc5sc6cc7ccccc7cc6c45)cc3c2)cc1 ; c1cc(-c2ccc(-c3cccc4ccccc34)cc2)cc(N(c2ccc3sc4cc5ccccc5cc4c3c2)c2ccc(-c3cc4ccccc4c4ccccc34)c3ccccc23)c1 ; c1ccc(-c2cc(N(c3ccc4cc(-c5cc6ccccc6c6ccccc56)ccc4c3)c3ccc4sc5cc6ccccc6cc5c4c3)ccc2-c2ccc3ccccc3c2)cc1]

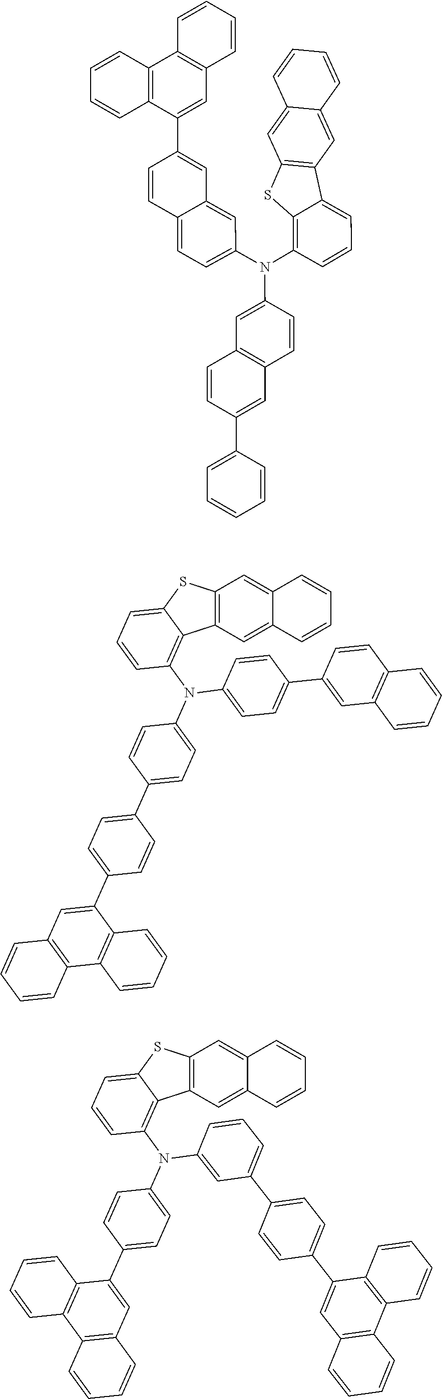 [SMILES: c1ccc(-c2ccc3cc(N(c4ccc5ccc(-c6cc7ccccc7c7ccccc67)cc5c4)c4cccc5c4sc4cc6ccccc6cc45)ccc3c2)cc1 ; c1ccc2cc(-c3ccc(N(c4ccc(-c5ccc(-c6cc7ccccc7c7ccccc67)cc5)cc4)c4cccc5sc6cc7ccccc7cc6c45)cc3)ccc2c1 ; c1cc(-c2ccc(-c3cc4ccccc4c4ccccc34)cc2)cc(N(c2ccc(-c3cc4ccccc4c4ccccc34)cc2)c2cccc3sc4cc5ccccc5cc4c23)c1]

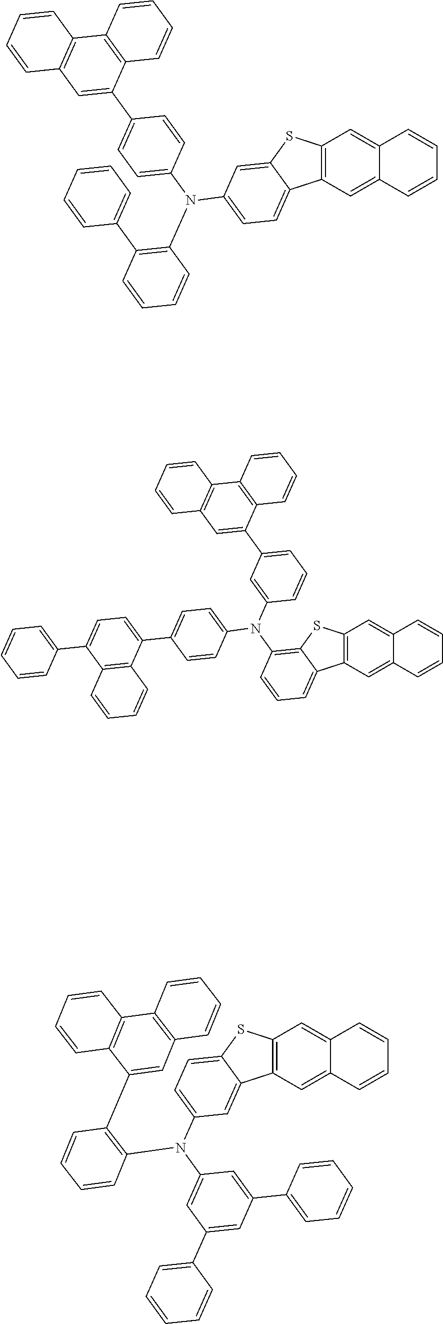 [SMILES: c1ccc(-c2ccccc2N(c2ccc(-c3cc4ccccc4c4ccccc34)cc2)c2ccc3c(c2)sc2cc4ccccc4cc23)cc1 ; c1ccc(-c2ccc(-c3ccc(N(c4cccc(-c5cc6ccccc6c6ccccc56)c4)c4cccc5c4sc4cc6ccccc6cc45)cc3)c3ccccc23)cc1 ; c1ccc(-c2cc(-c3ccccc3)cc(N(c3ccc4sc5cc6ccccc6cc5c4c3)c3ccccc3-c3cc4ccccc4c4ccccc34)c2)cc1]

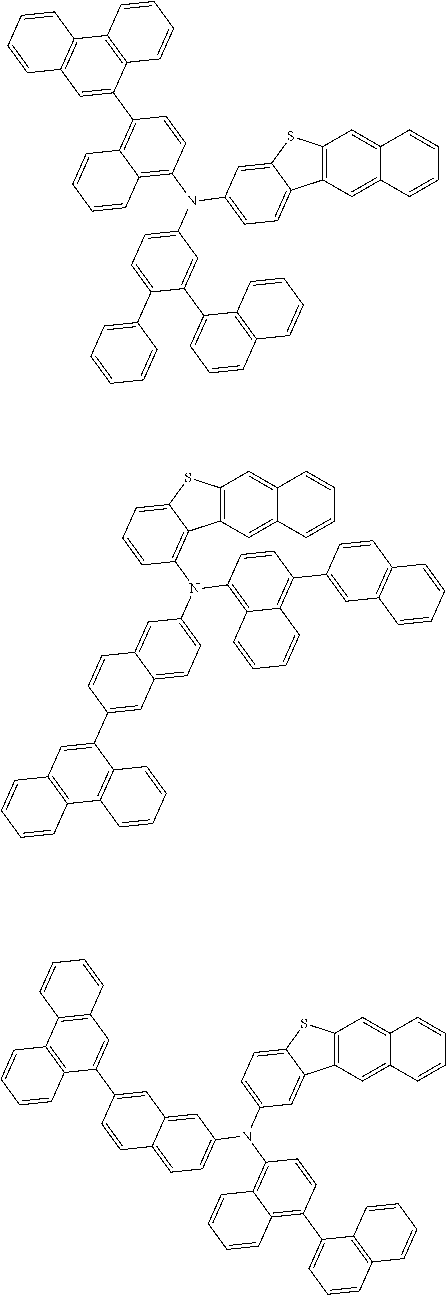 [SMILES: c1ccc(-c2ccc(N(c3ccc4c(c3)sc3cc5ccccc5cc34)c3ccc(-c4cc5ccccc5c5ccccc45)c4ccccc34)cc2-c2cccc3ccccc23)cc1 ; c1ccc2cc(-c3ccc(N(c4ccc5cc(-c6cc7ccccc7c7ccccc67)ccc5c4)c4cccc5sc6cc7ccccc7cc6c45)c4ccccc34)ccc2c1 ; c1ccc2cc3c(cc2c1)sc1ccc(N(c2ccc4ccc(-c5cc6ccccc6c6ccccc56)cc4c2)c2ccc(-c4cccc5ccccc45)c4ccccc24)cc13]

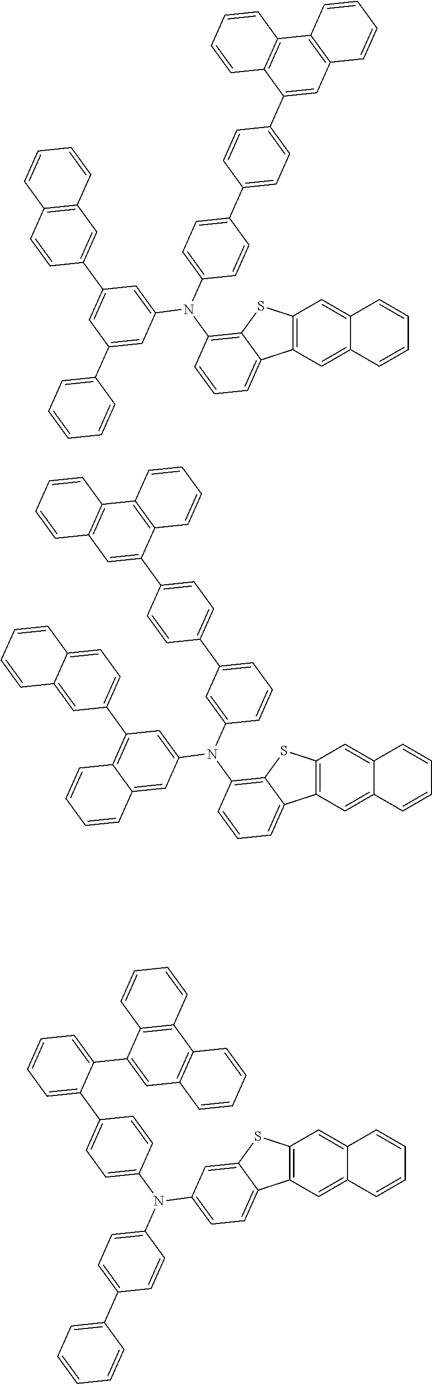

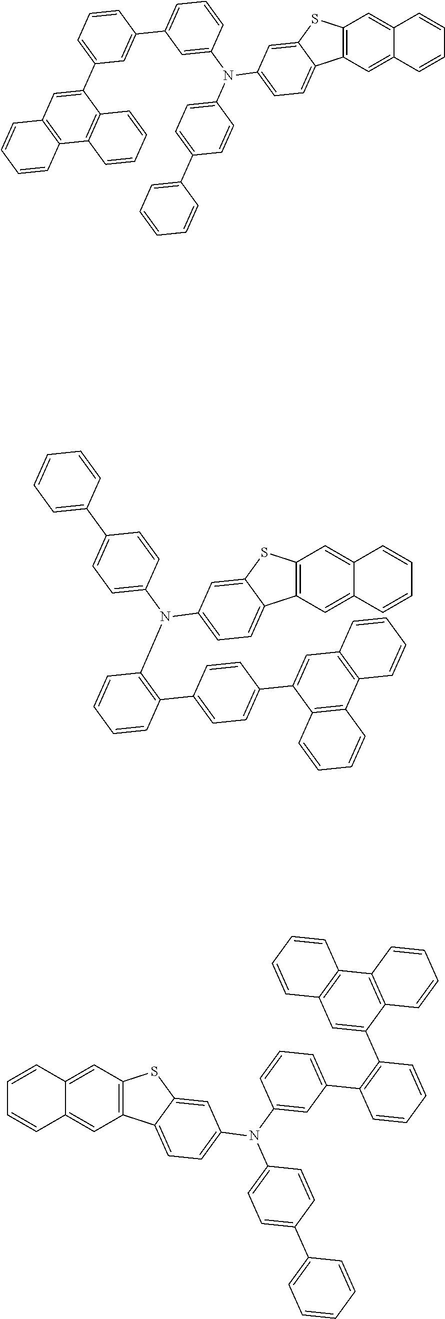 [SMILES: c1ccc(-c2ccc(N(c3cccc(-c4cccc(-c5cc6ccccc6c6ccccc56)c4)c3)c3ccc4c(c3)sc3cc5ccccc5cc34)cc2)cc1 ; c1ccc(-c2ccc(N(c3ccc4c(c3)sc3cc5ccccc5cc34)c3ccccc3-c3ccc(-c4cc5ccccc5c5ccccc45)cc3)cc2)cc1 ; c1ccc(-c2ccc(N(c3cccc(-c4ccccc4-c4cc5ccccc5c5ccccc45)c3)c3ccc4c(c3)sc3cc5ccccc5cc34)cc2)cc1]

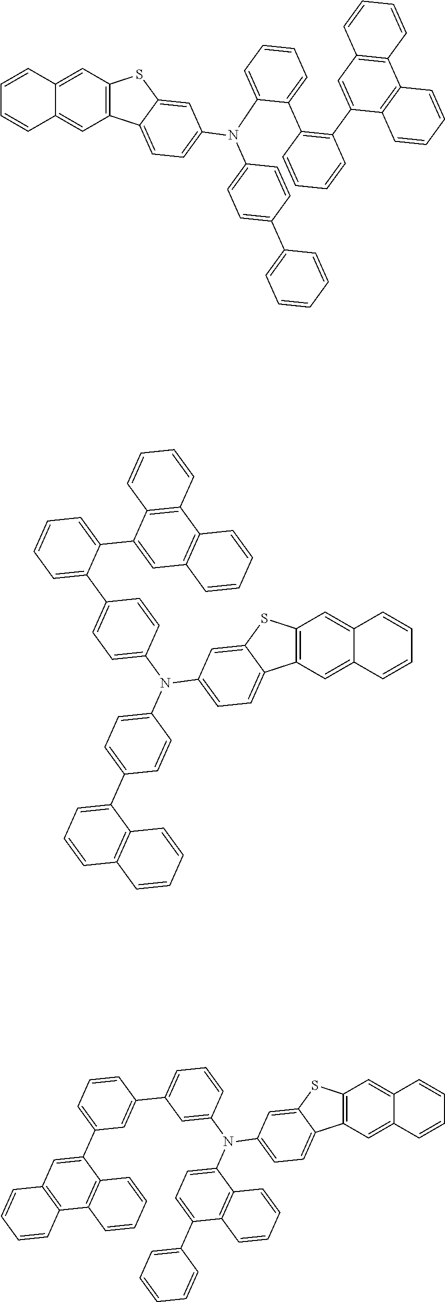 [SMILES: c1ccc(-c2ccc(N(c3ccc4c(c3)sc3cc5ccccc5cc34)c3ccccc3-c3ccccc3-c3cc4ccccc4c4ccccc34)cc2)cc1 ; c1ccc(-c2cc3ccccc3c3ccccc23)c(-c2ccc(N(c3ccc(-c4cccc5ccccc45)cc3)c3ccc4c(c3)sc3cc5ccccc5cc34)cc2)c1 ; c1ccc(-c2ccc(N(c3cccc(-c4cccc(-c5cc6ccccc6c6ccccc56)c4)c3)c3ccc4c(c3)sc3cc5ccccc5cc34)c3ccccc23)cc1]

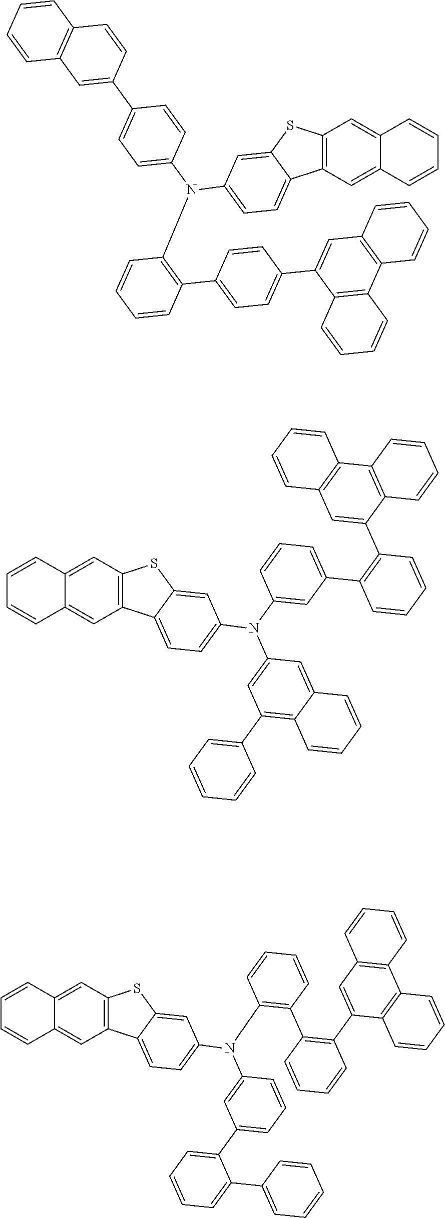 [SMILES: c1ccc(N(c2ccc(-c3ccc4ccccc4c3)cc2)c2ccc3c(c2)sc2cc4ccccc4cc23)c(-c2ccc(-c3cc4ccccc4c4ccccc34)cc2)c1 ; c1ccc(-c2cc(N(c3cccc(-c4ccccc4-c4cc5ccccc5c5ccccc45)c3)c3ccc4c(c3)sc3cc5ccccc5cc34)cc3ccccc23)cc1 ; c1ccc(-c2ccccc2-c2cccc(N(c3ccc4c(c3)sc3cc5ccccc5cc34)c3ccccc3-c3ccccc3-c3cc4ccccc4c4ccccc34)c2)cc1]

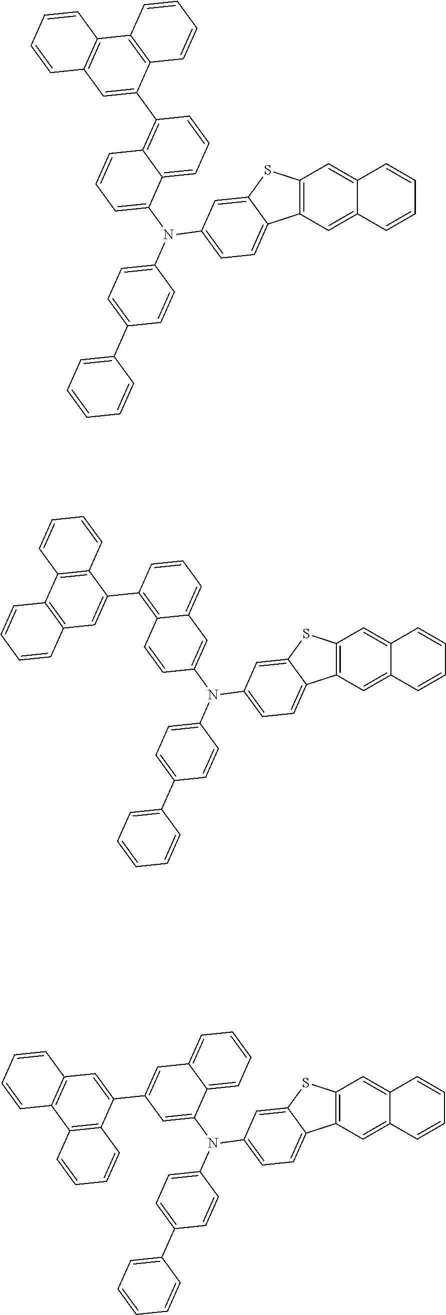 [SMILES: c1ccc(-c2ccc(N(c3ccc4c(c3)sc3cc5ccccc5cc34)c3cccc4c(-c5cc6ccccc6c6ccccc56)cccc34)cc2)cc1 ; c1ccc(-c2ccc(N(c3ccc4c(-c5cc6ccccc6c6ccccc56)cccc4c3)c3ccc4c(c3)sc3cc5ccccc5cc34)cc2)cc1 ; c1ccc(-c2ccc(N(c3ccc4c(c3)sc3cc5ccccc5cc34)c3cc(-c4cc5ccccc5c5ccccc45)cc4ccccc34)cc2)cc1]

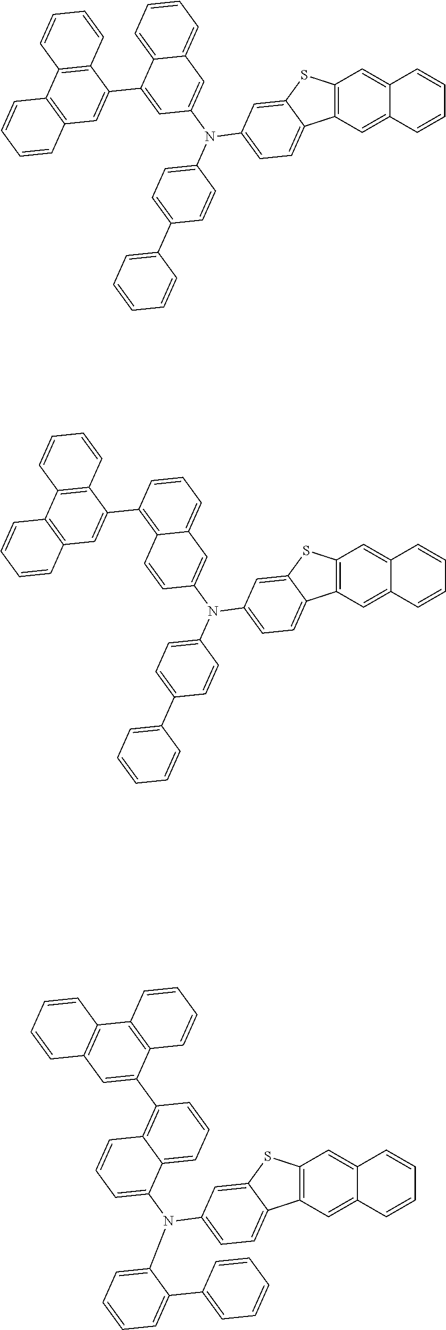 [SMILES: c1ccc(-c2ccc(N(c3cc(-c4cc5ccccc5c5ccccc45)c4ccccc4c3)c3ccc4c(c3)sc3cc5ccccc5cc34)cc2)cc1 ; c1ccc(-c2ccc(N(c3ccc4c(-c5cc6ccccc6c6ccccc56)cccc4c3)c3ccc4c(c3)sc3cc5ccccc5cc34)cc2)cc1 ; c1ccc(-c2ccccc2N(c2ccc3c(c2)sc2cc4ccccc4cc23)c2cccc3c(-c4cc5ccccc5c5ccccc45)cccc23)cc1]

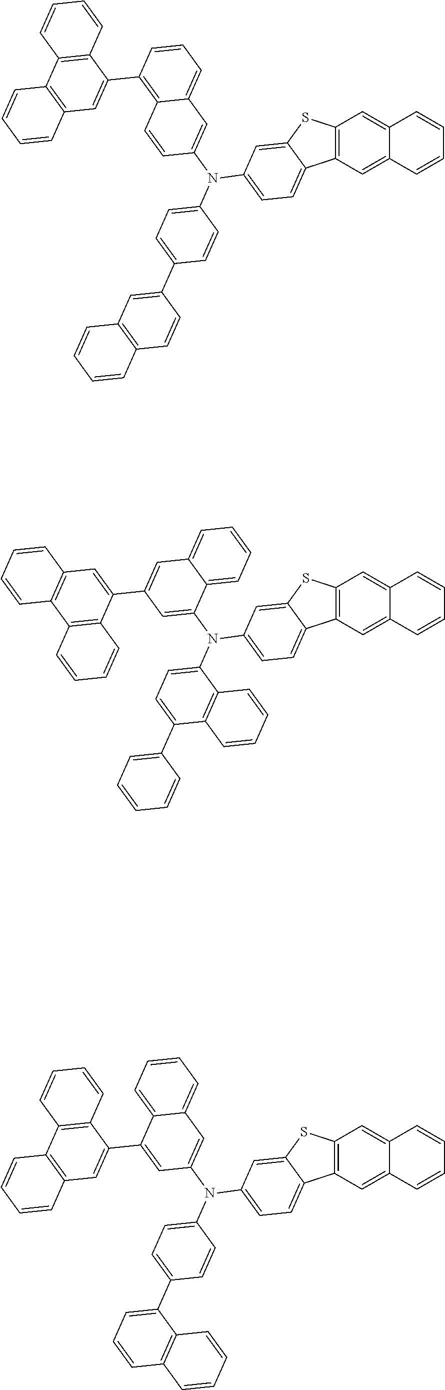 [SMILES: c1ccc2cc(-c3ccc(N(c4ccc5c(-c6cc7ccccc7c7ccccc67)cccc5c4)c4ccc5c(c4)sc4cc6ccccc6cc45)cc3)ccc2c1 ; c1ccc(-c2ccc(N(c3ccc4c(c3)sc3cc5ccccc5cc34)c3cc(-c4cc5ccccc5c5ccccc45)cc4ccccc34)c3ccccc23)cc1 ; c1ccc2cc3c(cc2c1)sc1cc(N(c2ccc(-c4cccc5ccccc45)cc2)c2cc(-c4cc5ccccc5c5ccccc45)c4ccccc4c2)ccc13]

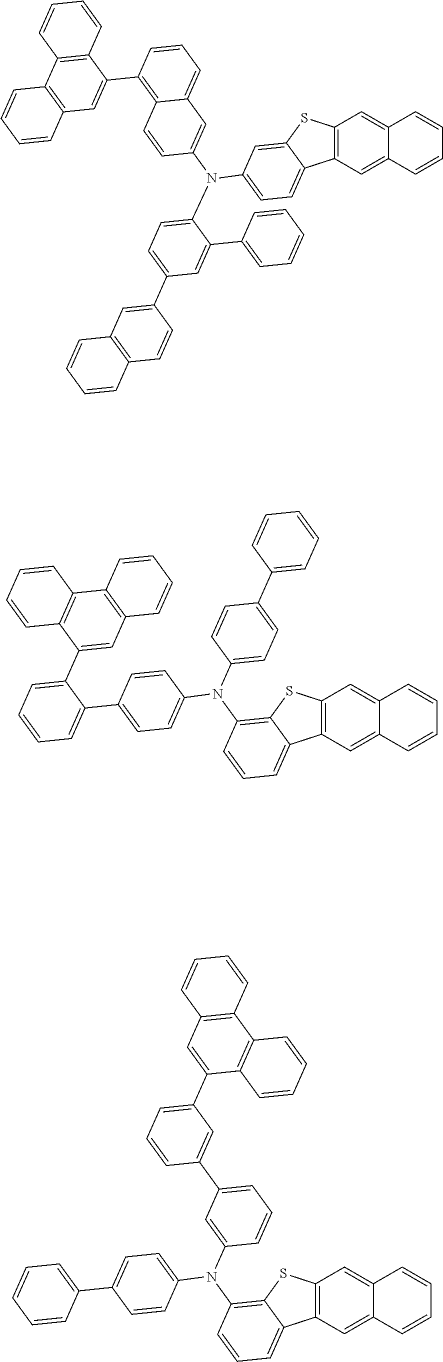 [SMILES: c1ccc(-c2cc(-c3ccc4ccccc4c3)ccc2N(c2ccc3c(-c4cc5ccccc5c5ccccc45)cccc3c2)c2ccc3c(c2)sc2cc4ccccc4cc23)cc1 ; c1ccc(-c2ccc(N(c3ccc(-c4ccccc4-c4cc5ccccc5c5ccccc45)cc3)c3cccc4c3sc3cc5ccccc5cc34)cc2)cc1 ; c1ccc(-c2ccc(N(c3cccc(-c4cccc(-c5cc6ccccc6c6ccccc56)c4)c3)c3cccc4c3sc3cc5ccccc5cc34)cc2)cc1]

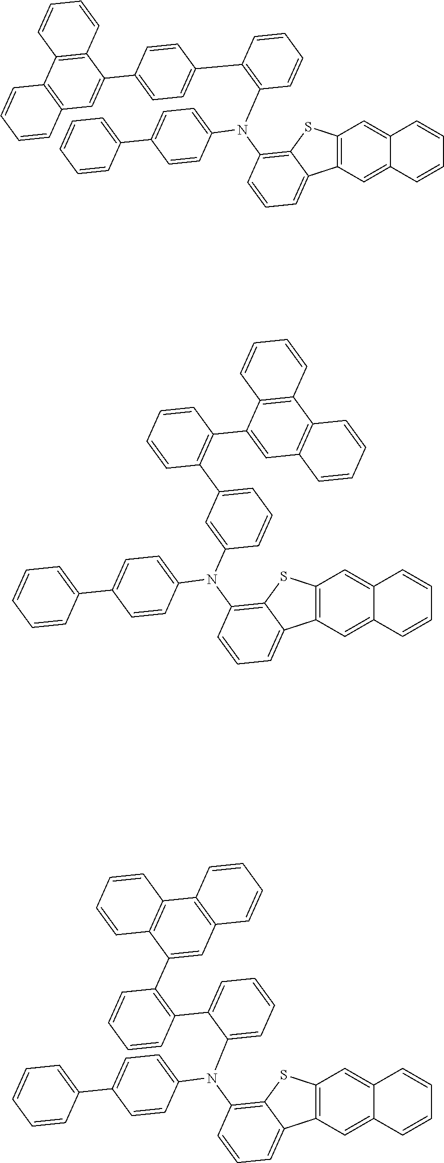 [SMILES: c1ccc(-c2ccc(N(c3ccccc3-c3ccc(-c4cc5ccccc5c5ccccc45)cc3)c3cccc4c3sc3cc5ccccc5cc34)cc2)cc1 ; c1ccc(-c2ccc(N(c3cccc(-c4ccccc4-c4cc5ccccc5c5ccccc45)c3)c3cccc4c3sc3cc5ccccc5cc34)cc2)cc1 ; c1ccc(-c2ccc(N(c3ccccc3-c3ccccc3-c3cc4ccccc4c4ccccc34)c3cccc4c3sc3cc5ccccc5cc34)cc2)cc1]

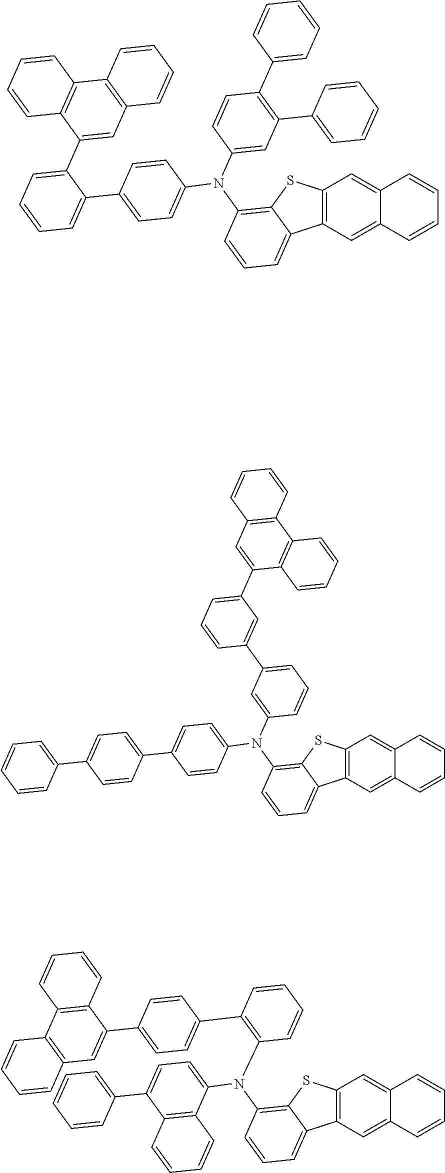 [SMILES: c1ccc(-c2ccc(N(c3ccc(-c4ccccc4-c4cc5ccccc5c5ccccc45)cc3)c3cccc4c3sc3cc5ccccc5cc34)cc2-c2ccccc2)cc1 ; c1ccc(-c2ccc(-c3ccc(N(c4cccc(-c5cccc(-c6cc7ccccc7c7ccccc67)c5)c4)c4cccc5c4sc4cc6ccccc6cc45)cc3)cc2)cc1 ; c1ccc(-c2ccc(N(c3ccccc3-c3ccc(-c4cc5ccccc5c5ccccc45)cc3)c3cccc4c3sc3cc5ccccc5cc34)c3ccccc23)cc1]

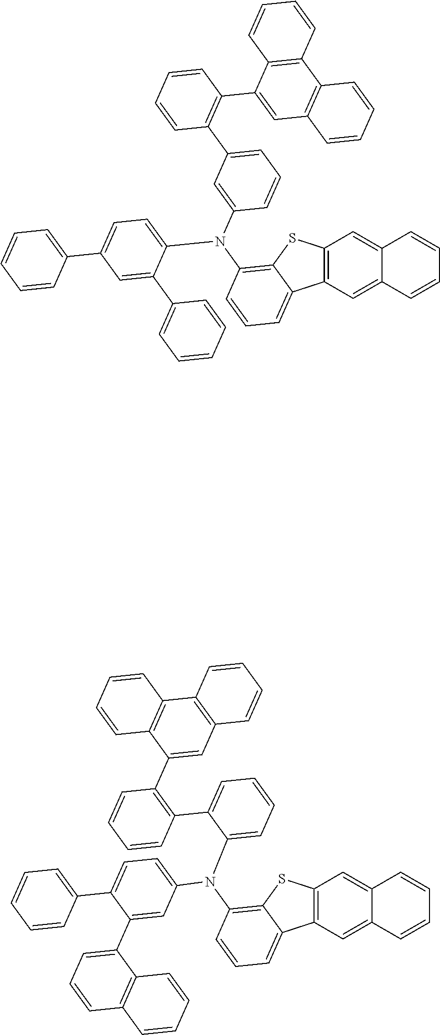 [SMILES: c1ccc(-c2ccc(N(c3cccc(-c4ccccc4-c4cc5ccccc5c5ccccc45)c3)c3cccc4c3sc3cc5ccccc5cc34)c(-c3ccccc3)c2)cc1 ; c1ccc(-c2ccc(N(c3ccccc3-c3ccccc3-c3cc4ccccc4c4ccccc34)c3cccc4c3sc3cc5ccccc5cc34)cc2-c2cccc3ccccc23)cc1]

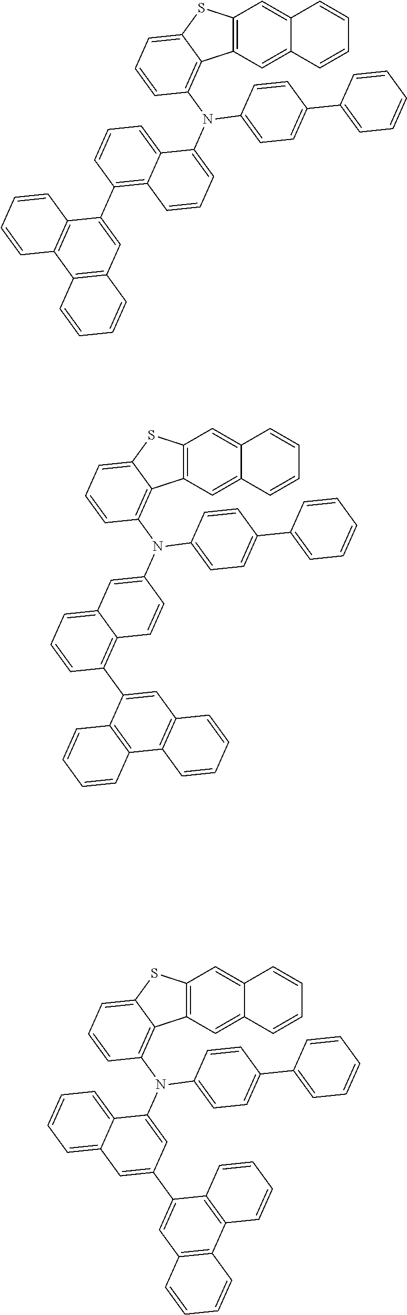 [SMILES: c1ccc(-c2ccc(N(c3cccc4c(-c5cc6ccccc6c6ccccc56)cccc34)c3cccc4sc5cc6ccccc6cc5c34)cc2)cc1 ; c1ccc(-c2ccc(N(c3ccc4c(-c5cc6ccccc6c6ccccc56)cccc4c3)c3cccc4sc5cc6ccccc6cc5c34)cc2)cc1 ; c1ccc(-c2ccc(N(c3cc(-c4cc5ccccc5c5ccccc45)cc4ccccc34)c3cccc4sc5cc6ccccc6cc5c34)cc2)cc1]

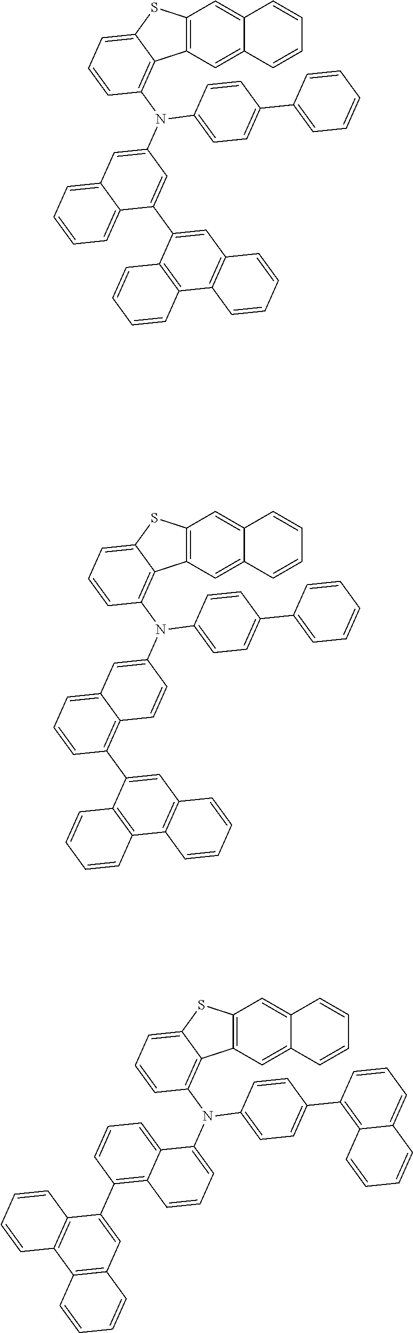 [SMILES: c1ccc(-c2ccc(N(c3cc(-c4cc5ccccc5c5ccccc45)c4ccccc4c3)c3cccc4sc5cc6ccccc6cc5c34)cc2)cc1 ; c1ccc(-c2ccc(N(c3ccc4c(-c5cc6ccccc6c6ccccc56)cccc4c3)c3cccc4sc5cc6ccccc6cc5c34)cc2)cc1 ; c1ccc2cc3c(cc2c1)sc1cccc(N(c2ccc(-c4cccc5ccccc45)cc2)c2cccc4c(-c5cc6ccccc6c6ccccc56)cccc24)c13]

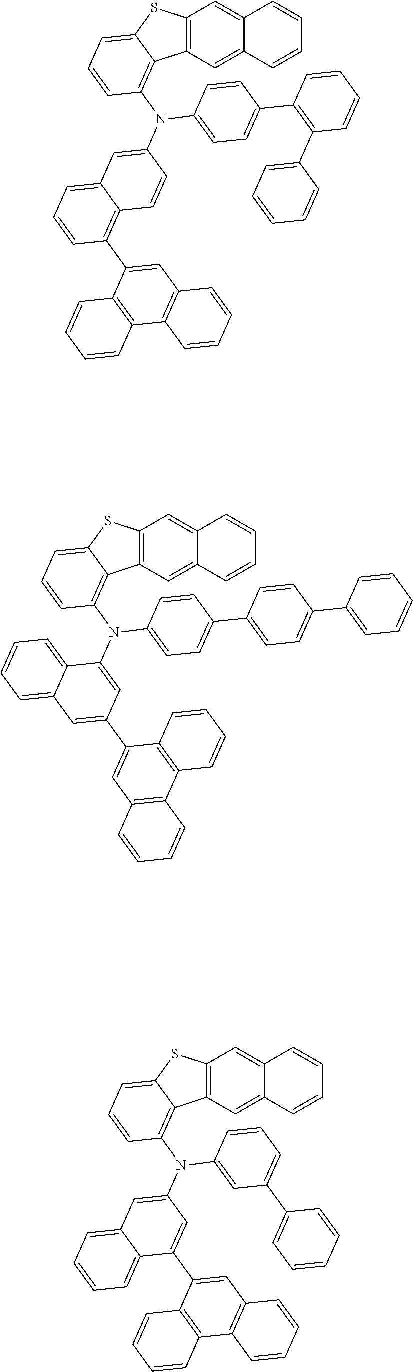 [SMILES: c1ccc(-c2ccccc2-c2ccc(N(c3ccc4c(-c5cc6ccccc6c6ccccc56)cccc4c3)c3cccc4sc5cc6ccccc6cc5c34)cc2)cc1 ; c1ccc(-c2ccc(-c3ccc(N(c4cc(-c5cc6ccccc6c6ccccc56)cc5ccccc45)c4cccc5sc6cc7ccccc7cc6c45)cc3)cc2)cc1 ; c1ccc(-c2cccc(N(c3cc(-c4cc5ccccc5c5ccccc45)c4ccccc4c3)c3cccc4sc5cc6ccccc6cc5c34)c2)cc1]

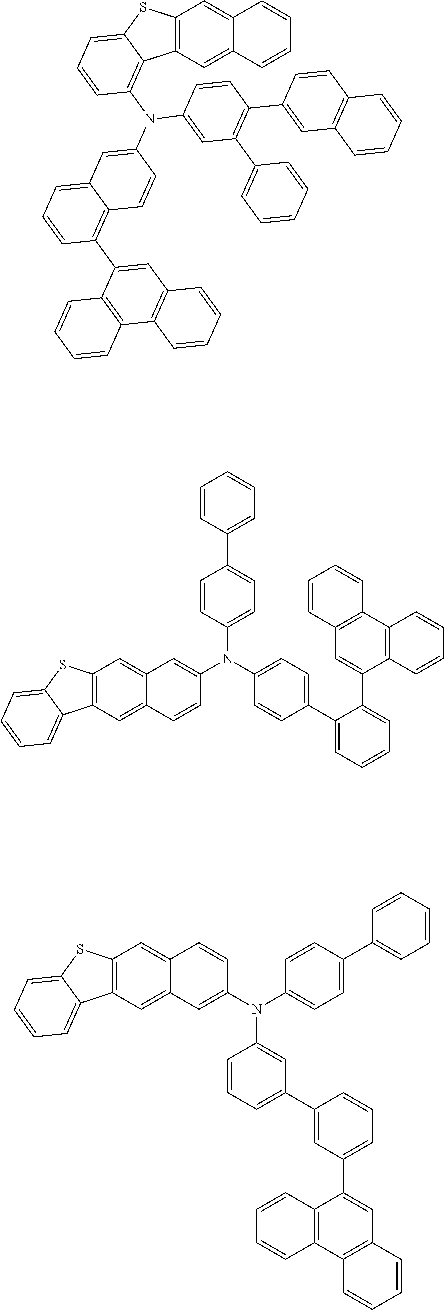 [SMILES: c1ccc(-c2cc(N(c3ccc4c(-c5cc6ccccc6c6ccccc56)cccc4c3)c3cccc4sc5cc6ccccc6cc5c34)ccc2-c2ccc3ccccc3c2)cc1 ; c1ccc(-c2ccc(N(c3ccc(-c4ccccc4-c4cc5ccccc5c5ccccc45)cc3)c3ccc4cc5c(cc4c3)sc3ccccc35)cc2)cc1 ; c1ccc(-c2ccc(N(c3cccc(-c4cccc(-c5cc6ccccc6c6ccccc56)c4)c3)c3ccc4cc5sc6ccccc6c5cc4c3)cc2)cc1]

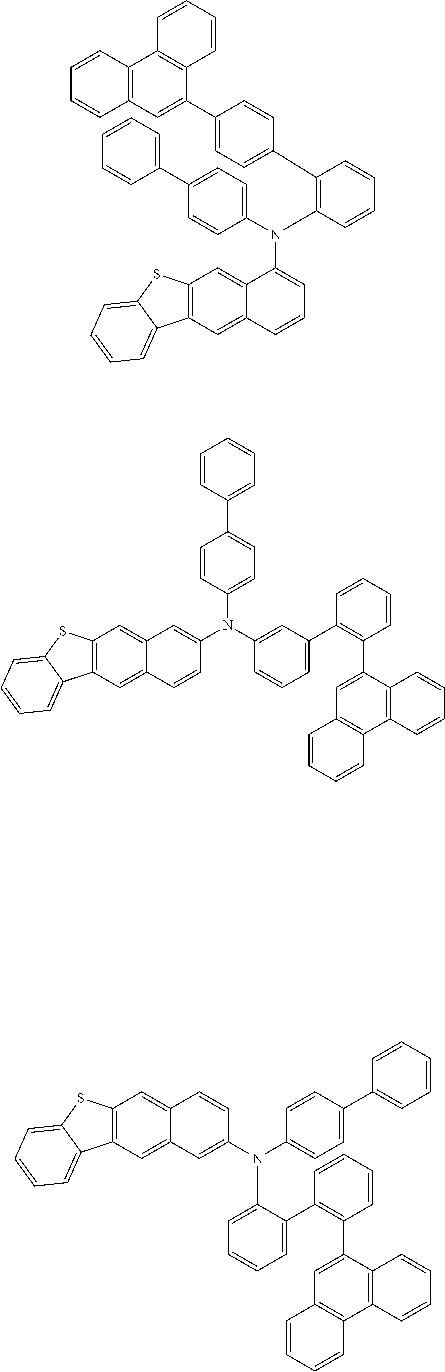 [SMILES: c1ccc(-c2ccc(N(c3ccccc3-c3ccc(-c4cc5ccccc5c5ccccc45)cc3)c3cccc4cc5c(cc34)sc3ccccc35)cc2)cc1 ; c1ccc(-c2ccc(N(c3cccc(-c4ccccc4-c4cc5ccccc5c5ccccc45)c3)c3ccc4cc5c(cc4c3)sc3ccccc35)cc2)cc1 ; c1ccc(-c2ccc(N(c3ccc4cc5sc6ccccc6c5cc4c3)c3ccccc3-c3ccccc3-c3cc4ccccc4c4ccccc34)cc2)cc1]

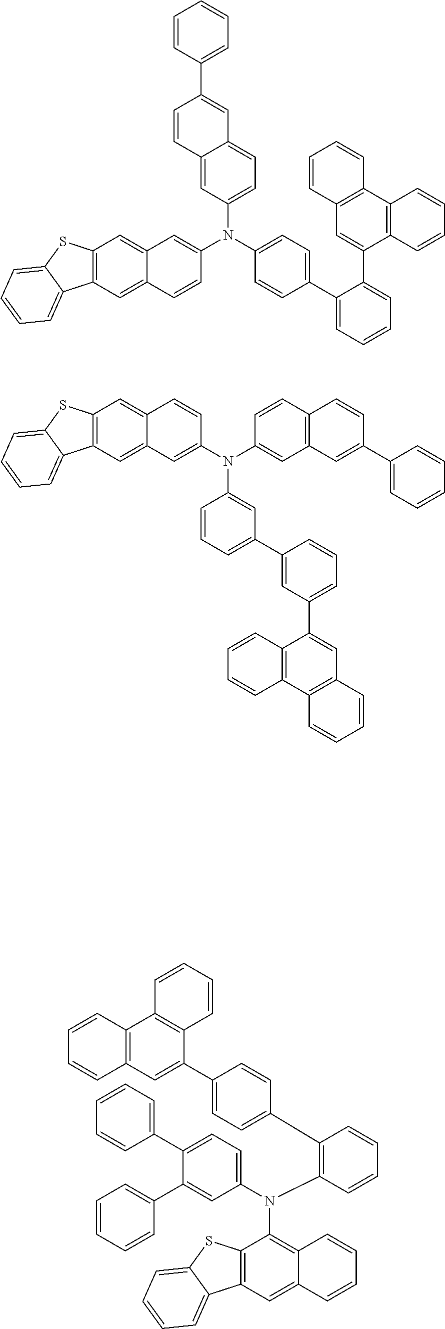 [SMILES: c1ccc(-c2ccc3cc(N(c4ccc(-c5ccccc5-c5cc6ccccc6c6ccccc56)cc4)c4ccc5cc6c(cc5c4)sc4ccccc46)ccc3c2)cc1 ; c1ccc(-c2ccc3ccc(N(c4cccc(-c5cccc(-c6cc7ccccc7c7ccccc67)c5)c4)c4ccc5cc6sc7ccccc7c6cc5c4)cc3c2)cc1 ; c1ccc(-c2ccc(N(c3ccccc3-c3ccc(-c4cc5ccccc5c5ccccc45)cc3)c3c4ccccc4cc4c3sc3ccccc34)cc2-c2ccccc2)cc1]

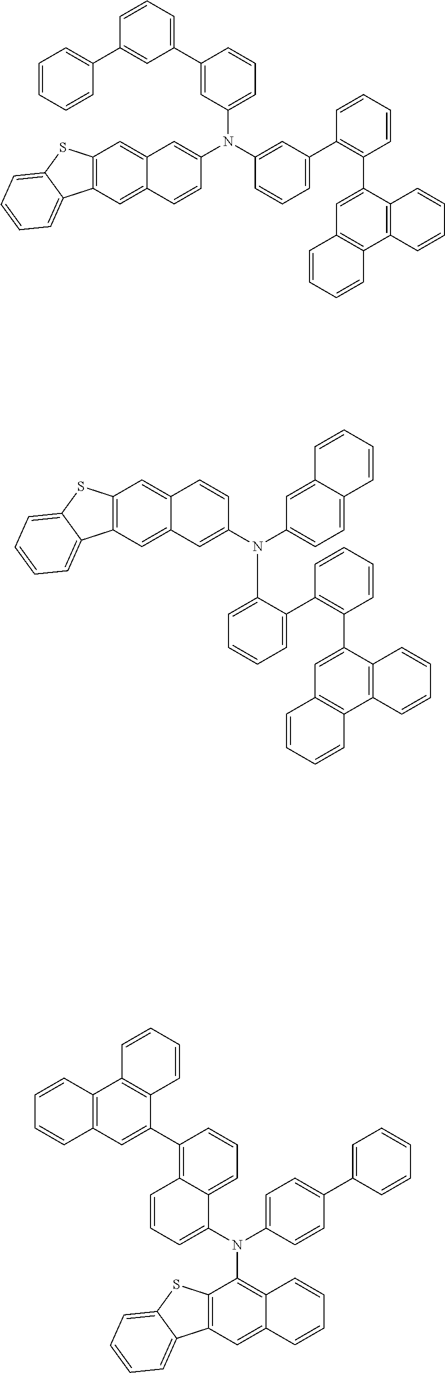 [SMILES: c1ccc(-c2cccc(-c3cccc(N(c4cccc(-c5ccccc5-c5cc6ccccc6c6ccccc56)c4)c4ccc5cc6c(cc5c4)sc4ccccc46)c3)c2)cc1 ; c1ccc(-c2cc3ccccc3c3ccccc23)c(-c2ccccc2N(c2ccc3ccccc3c2)c2ccc3cc4sc5ccccc5c4cc3c2)c1 ; c1ccc(-c2ccc(N(c3cccc4c(-c5cc6ccccc6c6ccccc56)cccc34)c3c4ccccc4cc4c3sc3ccccc34)cc2)cc1]

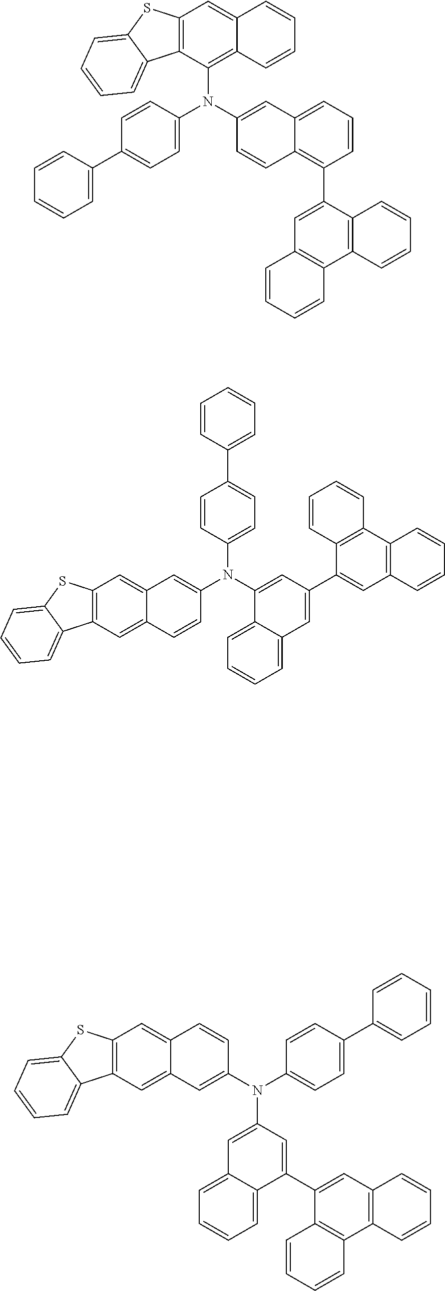 [SMILES: c1ccc(-c2ccc(N(c3ccc4c(-c5cc6ccccc6c6ccccc56)cccc4c3)c3c4ccccc4cc4sc5ccccc5c34)cc2)cc1 ; c1ccc(-c2ccc(N(c3ccc4cc5c(cc4c3)sc3ccccc35)c3cc(-c4cc5ccccc5c5ccccc45)cc4ccccc34)cc2)cc1 ; c1ccc(-c2ccc(N(c3ccc4cc5sc6ccccc6c5cc4c3)c3cc(-c4cc5ccccc5c5ccccc45)c4ccccc4c3)cc2)cc1]

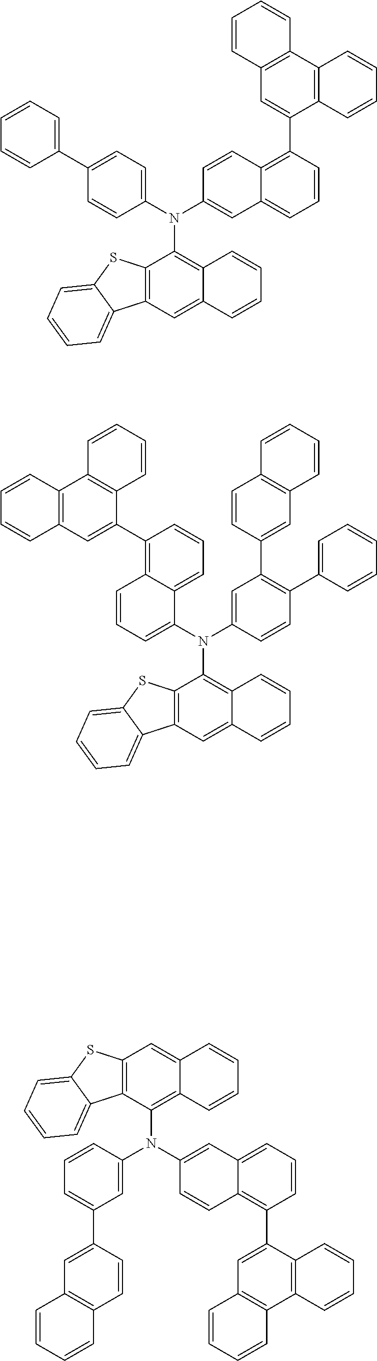 [SMILES: c1ccc(-c2ccc(N(c3ccc4c(-c5cc6ccccc6c6ccccc56)cccc4c3)c3c4ccccc4cc4c3sc3ccccc34)cc2)cc1 ; c1ccc(-c2ccc(N(c3cccc4c(-c5cc6ccccc6c6ccccc56)cccc34)c3c4ccccc4cc4c3sc3ccccc34)cc2-c2ccc3ccccc3c2)cc1 ; c1cc(-c2ccc3ccccc3c2)cc(N(c2ccc3c(-c4cc5ccccc5c5ccccc45)cccc3c2)c2c3ccccc3cc3sc4ccccc4c23)c1]

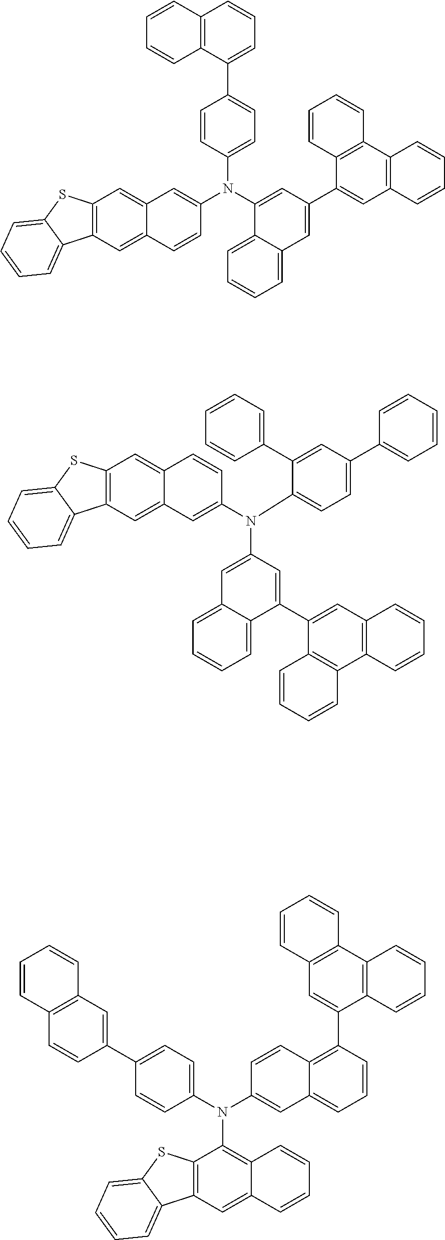 [SMILES: c1ccc2c(-c3ccc(N(c4ccc5cc6c(cc5c4)sc4ccccc46)c4cc(-c5cc6ccccc6c6ccccc56)cc5ccccc45)cc3)cccc2c1 ; c1ccc(-c2ccc(N(c3ccc4cc5sc6ccccc6c5cc4c3)c3cc(-c4cc5ccccc5c5ccccc45)c4ccccc4c3)c(-c3ccccc3)c2)cc1 ; c1ccc2cc(-c3ccc(N(c4ccc5c(-c6cc7ccccc7c7ccccc67)cccc5c4)c4c5ccccc5cc5c4sc4ccccc45)cc3)ccc2c1]

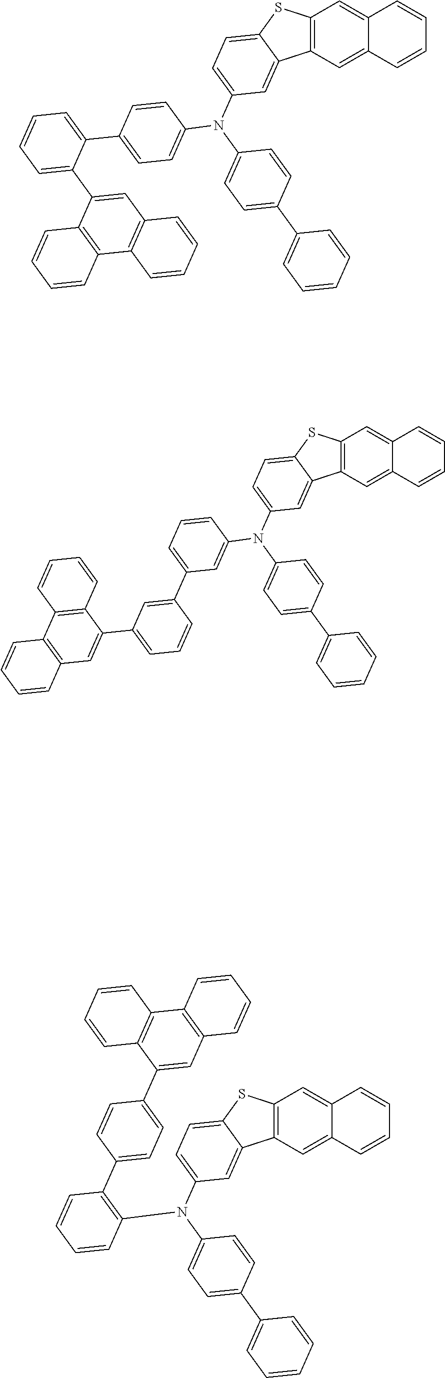 [SMILES: c1ccc(-c2ccc(N(c3ccc(-c4ccccc4-c4cc5ccccc5c5ccccc45)cc3)c3ccc4sc5cc6ccccc6cc5c4c3)cc2)cc1 ; c1ccc(-c2ccc(N(c3cccc(-c4cccc(-c5cc6ccccc6c6ccccc56)c4)c3)c3ccc4sc5cc6ccccc6cc5c4c3)cc2)cc1 ; c1ccc(-c2ccc(N(c3ccc4sc5cc6ccccc6cc5c4c3)c3ccccc3-c3ccc(-c4cc5ccccc5c5ccccc45)cc3)cc2)cc1]

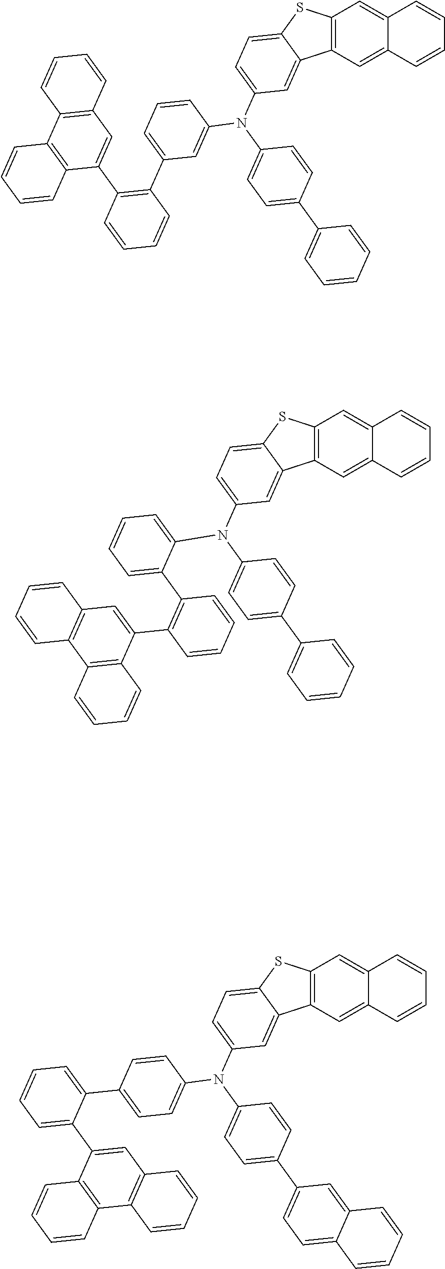 [SMILES: c1ccc(-c2ccc(N(c3cccc(-c4ccccc4-c4cc5ccccc5c5ccccc45)c3)c3ccc4sc5cc6ccccc6cc5c4c3)cc2)cc1 ; c1ccc(-c2ccc(N(c3ccc4sc5cc6ccccc6cc5c4c3)c3ccccc3-c3ccccc3-c3cc4ccccc4c4ccccc34)cc2)cc1 ; c1ccc(-c2cc3ccccc3c3ccccc23)c(-c2ccc(N(c3ccc(-c4ccc5ccccc5c4)cc3)c3ccc4sc5cc6ccccc6cc5c4c3)cc2)c1]

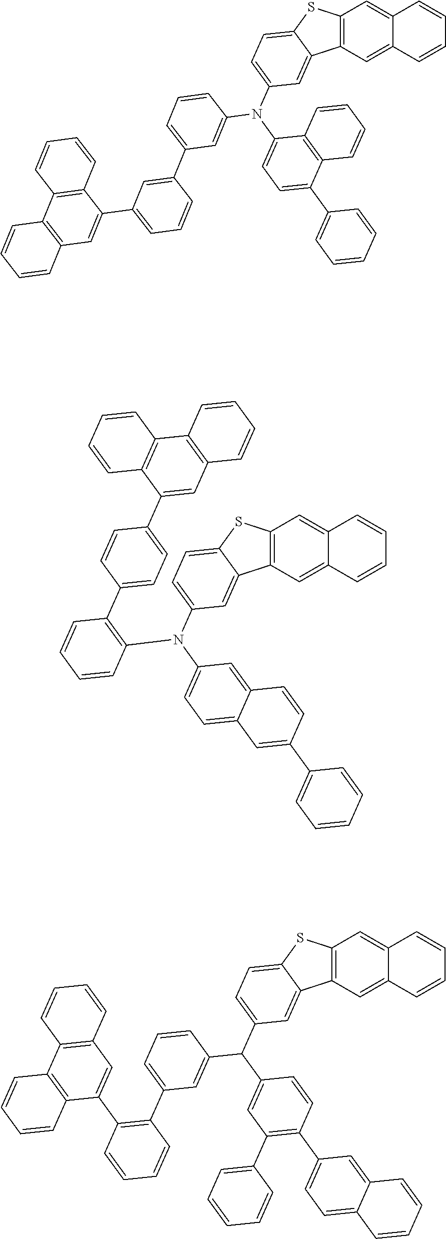 [SMILES: c1ccc(-c2ccc(N(c3cccc(-c4cccc(-c5cc6ccccc6c6ccccc56)c4)c3)c3ccc4sc5cc6ccccc6cc5c4c3)c3ccccc23)cc1 ; c1ccc(-c2ccc3cc(N(c4ccc5sc6cc7ccccc7cc6c5c4)c4ccccc4-c4ccc(-c5cc6ccccc6c6ccccc56)cc4)ccc3c2)cc1 ; c1ccc(-c2cc(C(c3cccc(-c4ccccc4-c4cc5ccccc5c5ccccc45)c3)c3ccc4sc5cc6ccccc6cc5c4c3)ccc2-c2ccc3ccccc3c2)cc1]

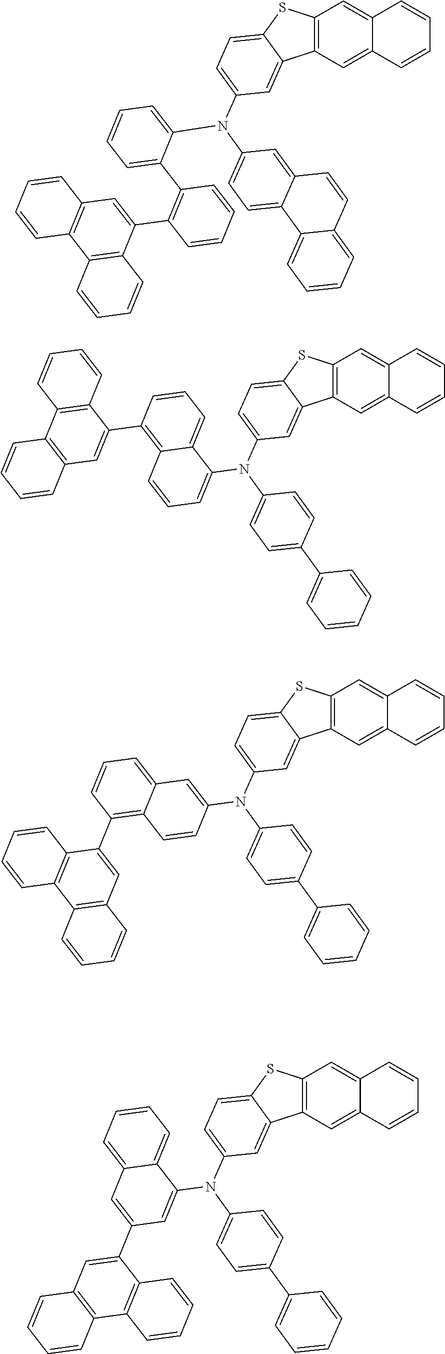

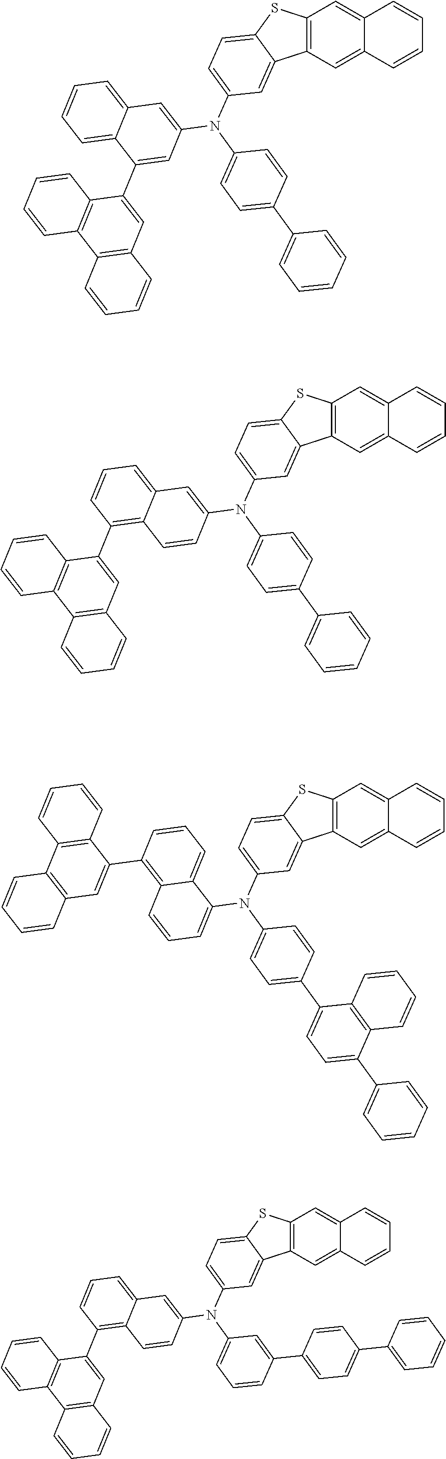 [SMILES: c1ccc(-c2ccc(N(c3cc(-c4cc5ccccc5c5ccccc45)c4ccccc4c3)c3ccc4sc5cc6ccccc6cc5c4c3)cc2)cc1 ; c1ccc(-c2ccc(N(c3ccc4c(-c5cc6ccccc6c6ccccc56)cccc4c3)c3ccc4sc5cc6ccccc6cc5c4c3)cc2)cc1 ; c1ccc(-c2ccc(-c3ccc(N(c4ccc5sc6cc7ccccc7cc6c5c4)c4cccc5c(-c6cc7ccccc7c7ccccc67)cccc45)cc3)c3ccccc23)cc1 ; c1ccc(-c2ccc(-c3cccc(N(c4ccc5c(-c6cc7ccccc7c7ccccc67)cccc5c4)c4ccc5sc6cc7ccccc7cc6c5c4)c3)cc2)cc1]

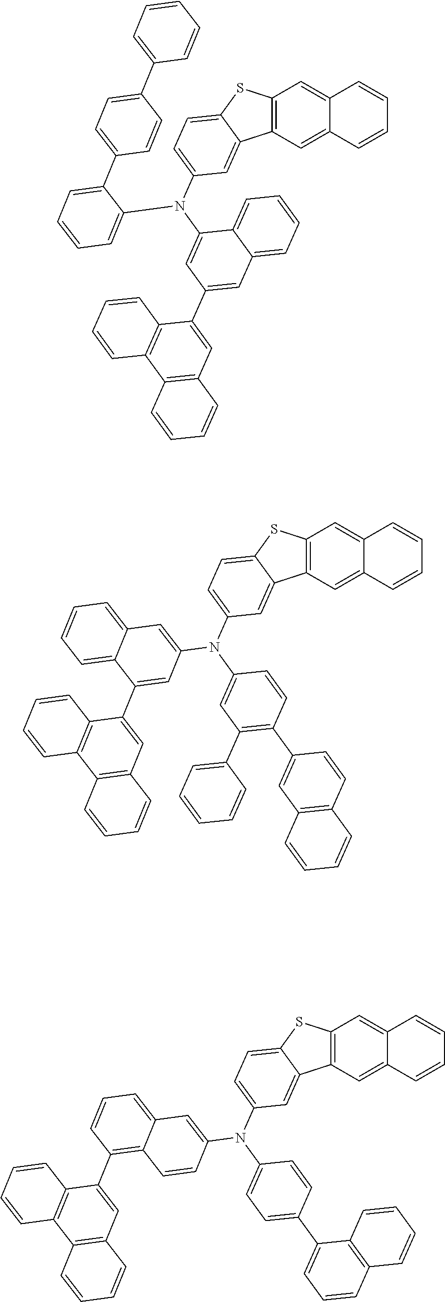 [SMILES: c1ccc(-c2ccc(-c3ccccc3N(c3ccc4sc5cc6ccccc6cc5c4c3)c3cc(-c4cc5ccccc5c5ccccc45)cc4ccccc34)cc2)cc1 ; c1ccc(-c2cc(N(c3cc(-c4cc5ccccc5c5ccccc45)c4ccccc4c3)c3ccc4sc5cc6ccccc6cc5c4c3)ccc2-c2ccc3ccccc3c2)cc1 ; c1ccc2cc3c(cc2c1)sc1ccc(N(c2ccc(-c4cccc5ccccc45)cc2)c2ccc4c(-c5cc6ccccc6c6ccccc56)cccc4c2)cc13]

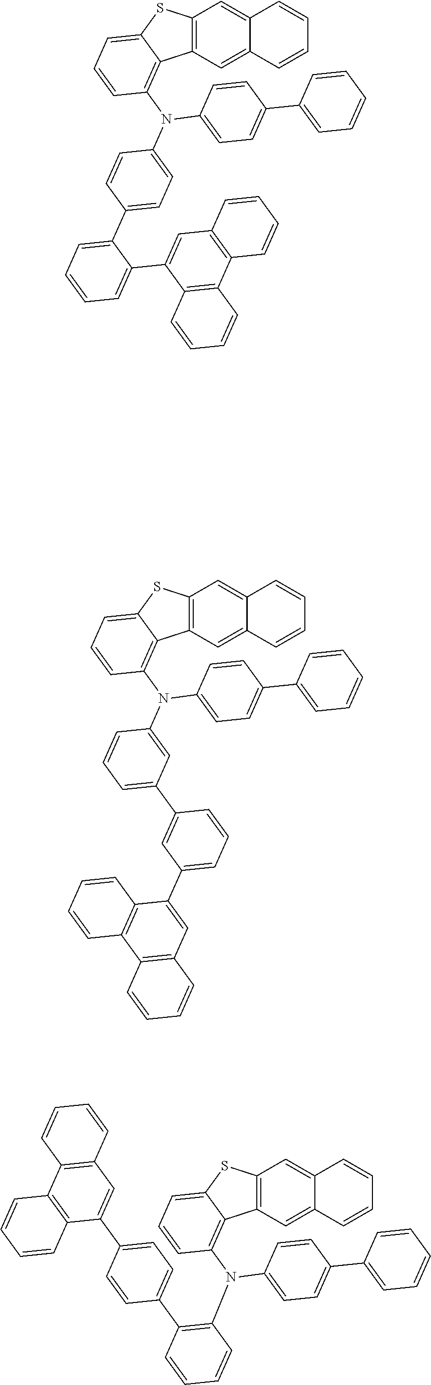 [SMILES: c1ccc(-c2ccc(N(c3ccc(-c4ccccc4-c4cc5ccccc5c5ccccc45)cc3)c3cccc4sc5cc6ccccc6cc5c34)cc2)cc1 ; c1ccc(-c2ccc(N(c3cccc(-c4cccc(-c5cc6ccccc6c6ccccc56)c4)c3)c3cccc4sc5cc6ccccc6cc5c34)cc2)cc1 ; c1ccc(-c2ccc(N(c3ccccc3-c3ccc(-c4cc5ccccc5c5ccccc45)cc3)c3cccc4sc5cc6ccccc6cc5c34)cc2)cc1]

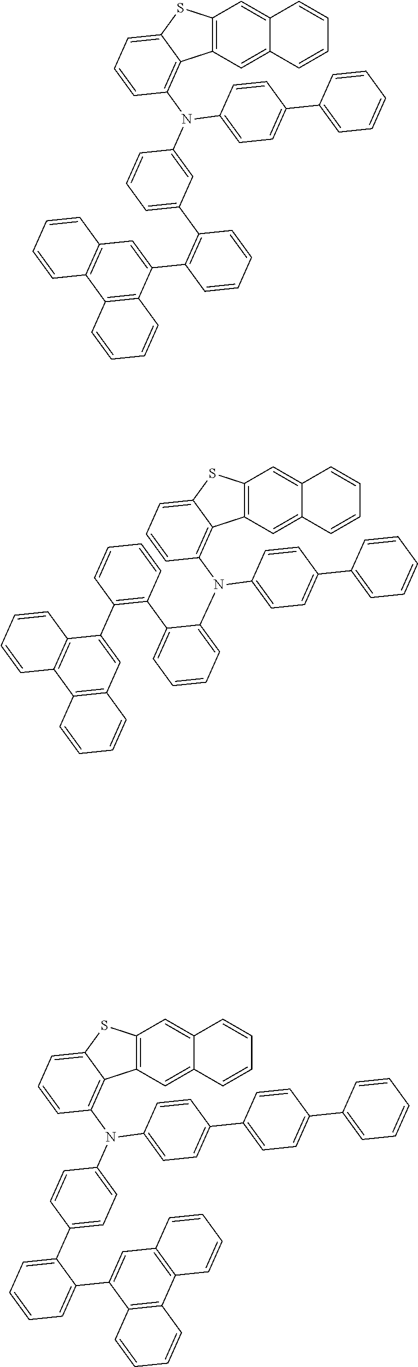 [SMILES: c1ccc(-c2ccc(N(c3cccc(-c4ccccc4-c4cc5ccccc5c5ccccc45)c3)c3cccc4sc5cc6ccccc6cc5c34)cc2)cc1 ; c1ccc(-c2ccc(N(c3ccccc3-c3ccccc3-c3cc4ccccc4c4ccccc34)c3cccc4sc5cc6ccccc6cc5c34)cc2)cc1 ; c1ccc(-c2ccc(-c3ccc(N(c4ccc(-c5ccccc5-c5cc6ccccc6c6ccccc56)cc4)c4cccc5sc6cc7ccccc7cc6c45)cc3)cc2)cc1]

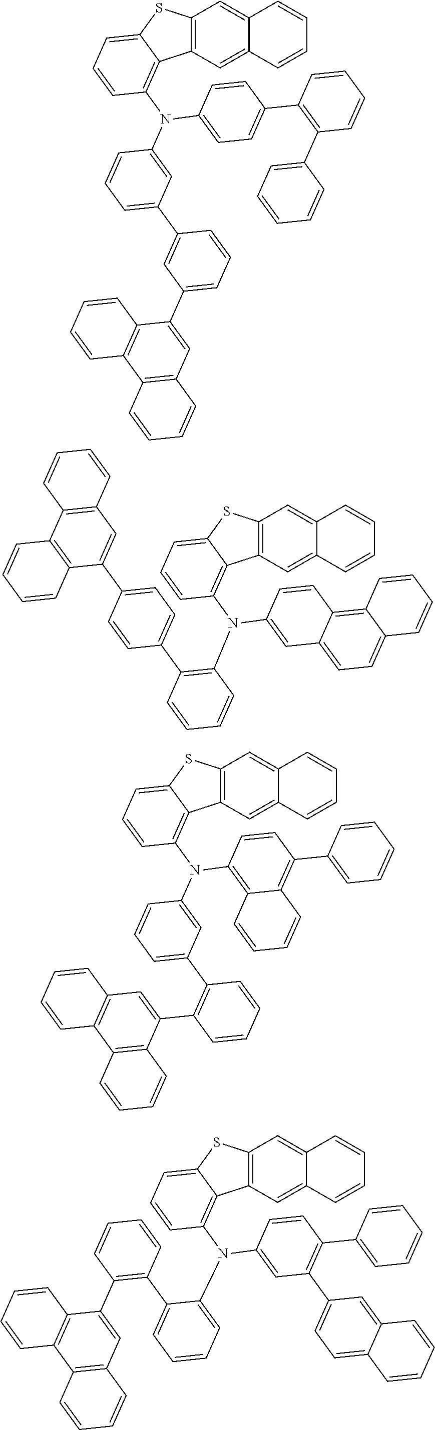

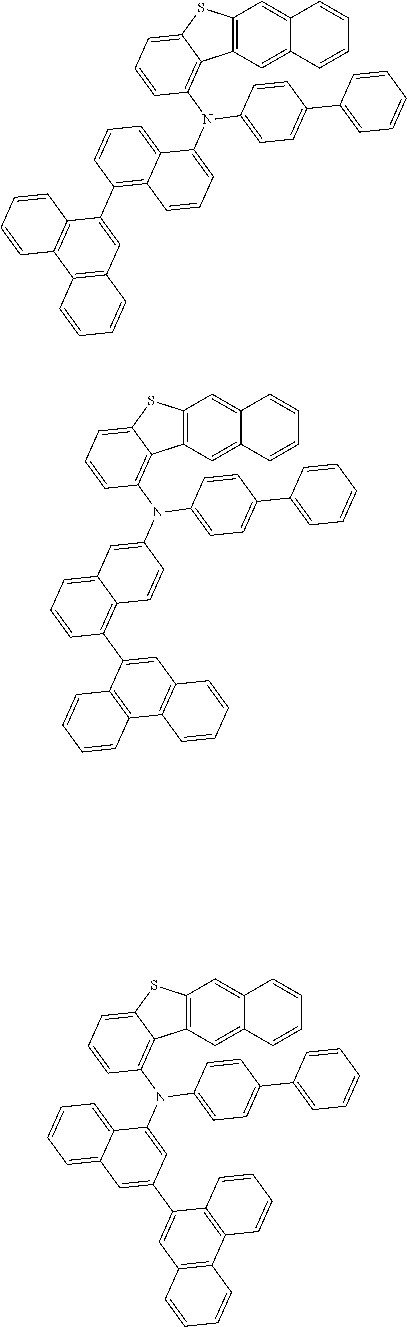 [SMILES: c1ccc(-c2ccc(N(c3cccc4c(-c5cc6ccccc6c6ccccc56)cccc34)c3cccc4sc5cc6ccccc6cc5c34)cc2)cc1 ; c1ccc(-c2ccc(N(c3ccc4c(-c5cc6ccccc6c6ccccc56)cccc4c3)c3cccc4sc5cc6ccccc6cc5c34)cc2)cc1 ; c1ccc(-c2ccc(N(c3cc(-c4cc5ccccc5c5ccccc45)cc4ccccc34)c3cccc4sc5cc6ccccc6cc5c34)cc2)cc1]

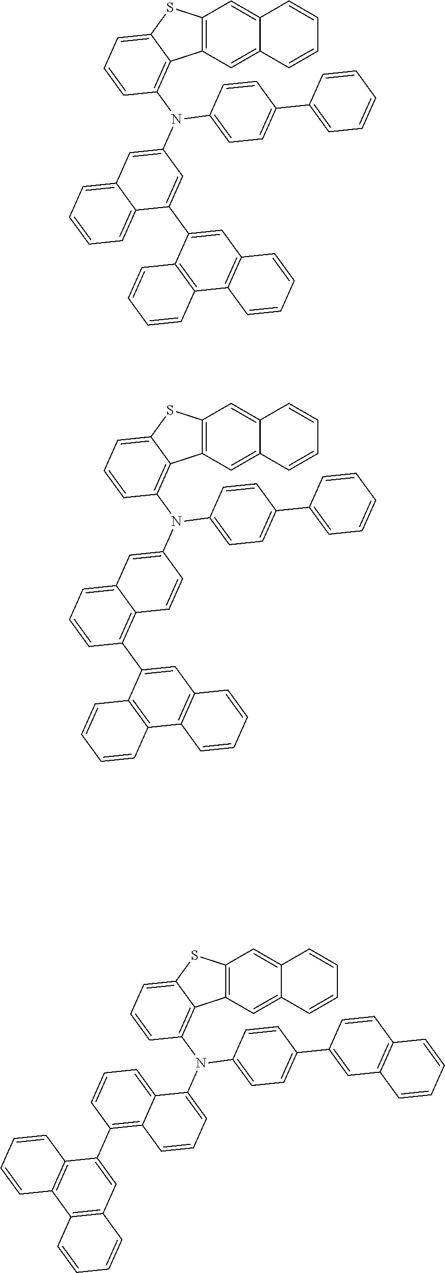 [SMILES: c1ccc(-c2ccc(N(c3cc(-c4cc5ccccc5c5ccccc45)c4ccccc4c3)c3cccc4sc5cc6ccccc6cc5c34)cc2)cc1 ; c1ccc(-c2ccc(N(c3ccc4c(-c5cc6ccccc6c6ccccc56)cccc4c3)c3cccc4sc5cc6ccccc6cc5c34)cc2)cc1 ; c1ccc2cc(-c3ccc(N(c4cccc5c(-c6cc7ccccc7c7ccccc67)cccc45)c4cccc5sc6cc7ccccc7cc6c45)cc3)ccc2c1]

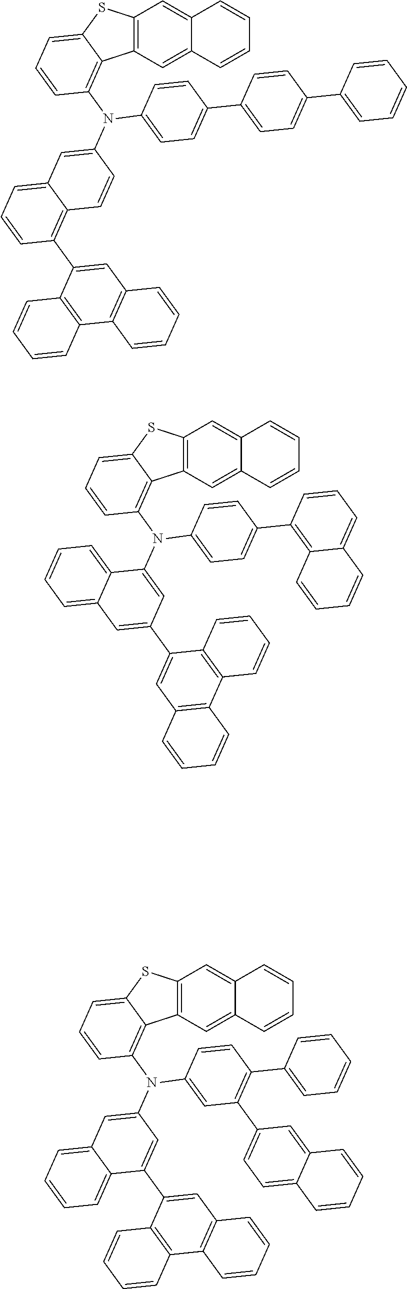 [SMILES: c1ccc(-c2ccc(-c3ccc(N(c4ccc5c(-c6cc7ccccc7c7ccccc67)cccc5c4)c4cccc5sc6cc7ccccc7cc6c45)cc3)cc2)cc1 ; c1ccc2cc3c(cc2c1)sc1cccc(N(c2ccc(-c4cccc5ccccc45)cc2)c2cc(-c4cc5ccccc5c5ccccc45)cc4ccccc24)c13 ; c1ccc(-c2ccc(N(c3cc(-c4cc5ccccc5c5ccccc45)c4ccccc4c3)c3cccc4sc5cc6ccccc6cc5c34)cc2-c2ccc3ccccc3c2)cc1]

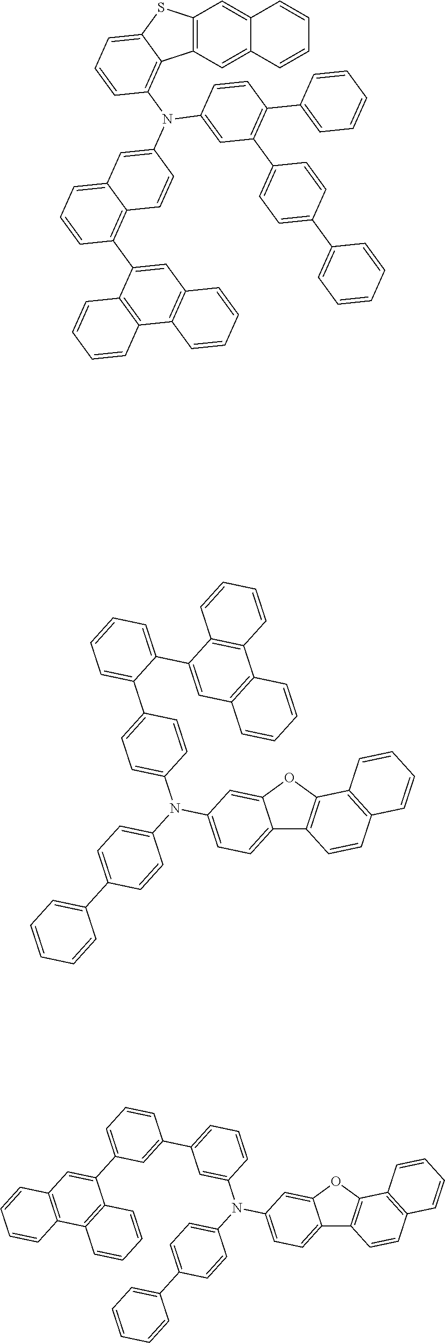 [SMILES: c1ccc(-c2ccc(-c3cc(N(c4ccc5c(-c6cc7ccccc7c7ccccc67)cccc5c4)c4cccc5sc6cc7ccccc7cc6c45)ccc3-c3ccccc3)cc2)cc1 ; c1ccc(-c2ccc(N(c3ccc(-c4ccccc4-c4cc5ccccc5c5ccccc45)cc3)c3ccc4c(c3)oc3c5ccccc5ccc43)cc2)cc1 ; c1ccc(-c2ccc(N(c3cccc(-c4cccc(-c5cc6ccccc6c6ccccc56)c4)c3)c3ccc4c(c3)oc3c5ccccc5ccc43)cc2)cc1]

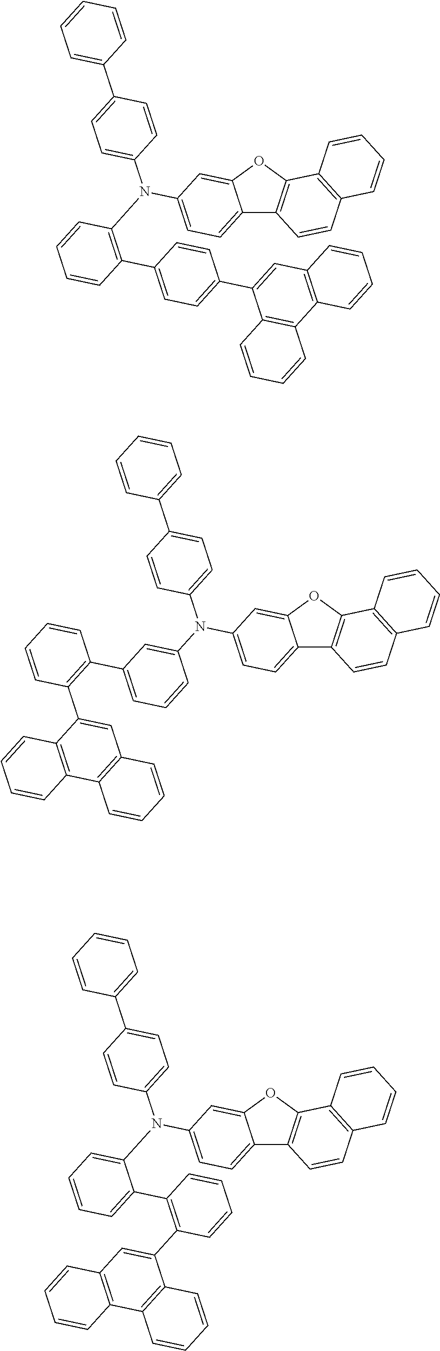 [SMILES: c1ccc(-c2ccc(N(c3ccc4c(c3)oc3c5ccccc5ccc43)c3ccccc3-c3ccc(-c4cc5ccccc5c5ccccc45)cc3)cc2)cc1 ; c1ccc(-c2ccc(N(c3cccc(-c4ccccc4-c4cc5ccccc5c5ccccc45)c3)c3ccc4c(c3)oc3c5ccccc5ccc43)cc2)cc1 ; c1ccc(-c2ccc(N(c3ccc4c(c3)oc3c5ccccc5ccc43)c3ccccc3-c3ccccc3-c3cc4ccccc4c4ccccc34)cc2)cc1]

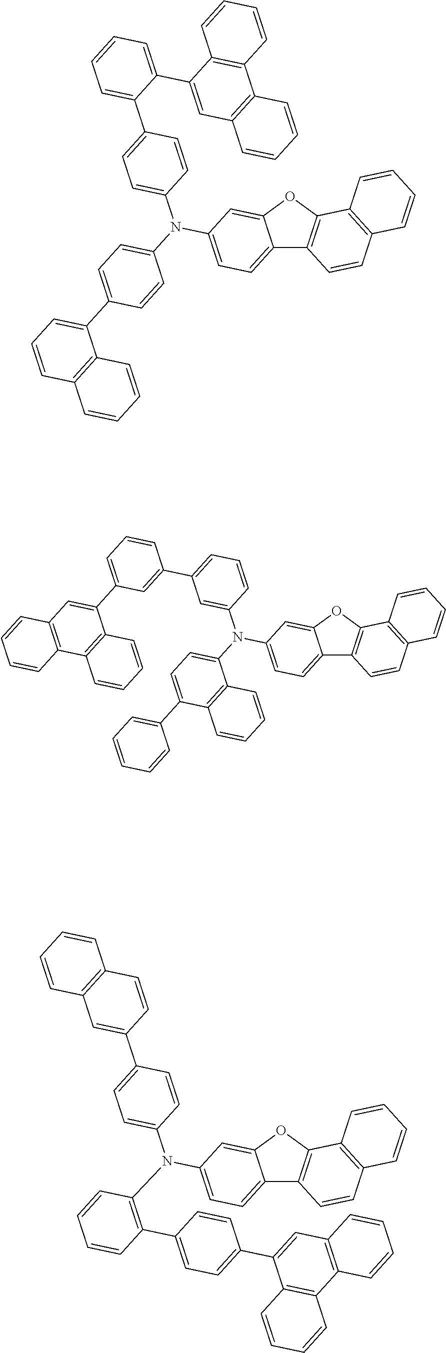 [SMILES: c1ccc(-c2cc3ccccc3c3ccccc23)c(-c2ccc(N(c3ccc(-c4cccc5ccccc45)cc3)c3ccc4c(c3)oc3c5ccccc5ccc43)cc2)c1 ; c1ccc(-c2ccc(N(c3cccc(-c4cccc(-c5cc6ccccc6c6ccccc56)c4)c3)c3ccc4c(c3)oc3c5ccccc5ccc43)c3ccccc23)cc1 ; c1ccc(N(c2ccc(-c3ccc4ccccc4c3)cc2)c2ccc3c(c2)oc2c4ccccc4ccc32)c(-c2ccc(-c3cc4ccccc4c4ccccc34)cc2)c1]

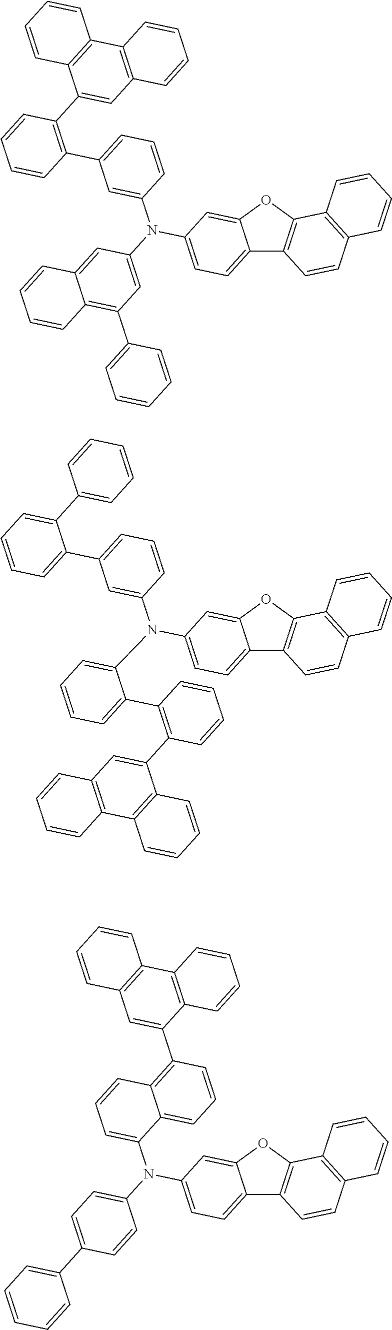 [SMILES: c1ccc(-c2cc(N(c3cccc(-c4ccccc4-c4cc5ccccc5c5ccccc45)c3)c3ccc4c(c3)oc3c5ccccc5ccc43)cc3ccccc23)cc1 ; c1ccc(-c2ccccc2-c2cccc(N(c3ccc4c(c3)oc3c5ccccc5ccc43)c3ccccc3-c3ccccc3-c3cc4ccccc4c4ccccc34)c2)cc1 ; c1ccc(-c2ccc(N(c3ccc4c(c3)oc3c5ccccc5ccc43)c3cccc4c(-c5cc6ccccc6c6ccccc56)cccc34)cc2)cc1]

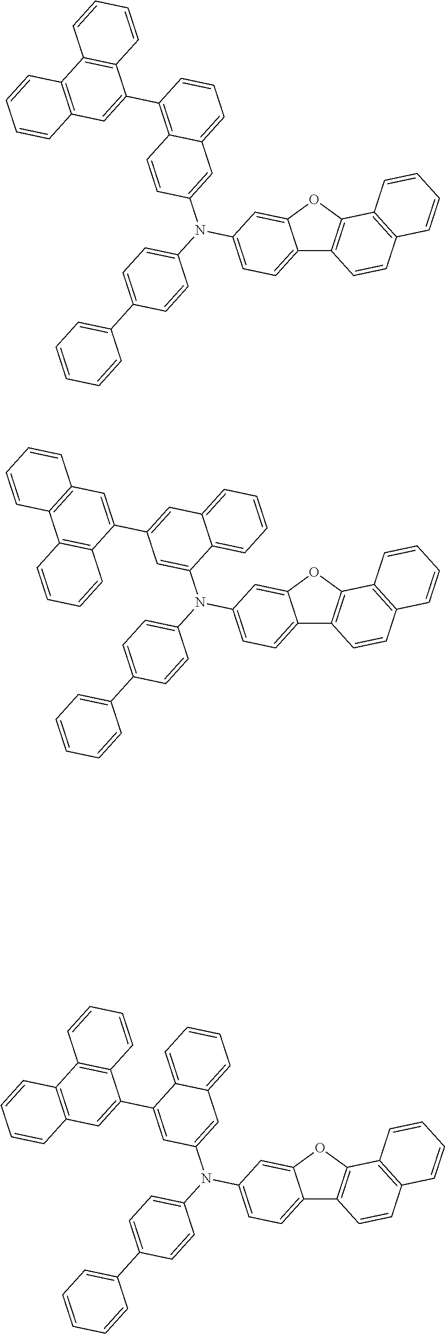 [SMILES: c1ccc(-c2ccc(N(c3ccc4c(-c5cc6ccccc6c6ccccc56)cccc4c3)c3ccc4c(c3)oc3c5ccccc5ccc43)cc2)cc1 ; c1ccc(-c2ccc(N(c3ccc4c(c3)oc3c5ccccc5ccc43)c3cc(-c4cc5ccccc5c5ccccc45)cc4ccccc34)cc2)cc1 ; c1ccc(-c2ccc(N(c3cc(-c4cc5ccccc5c5ccccc45)c4ccccc4c3)c3ccc4c(c3)oc3c5ccccc5ccc43)cc2)cc1]

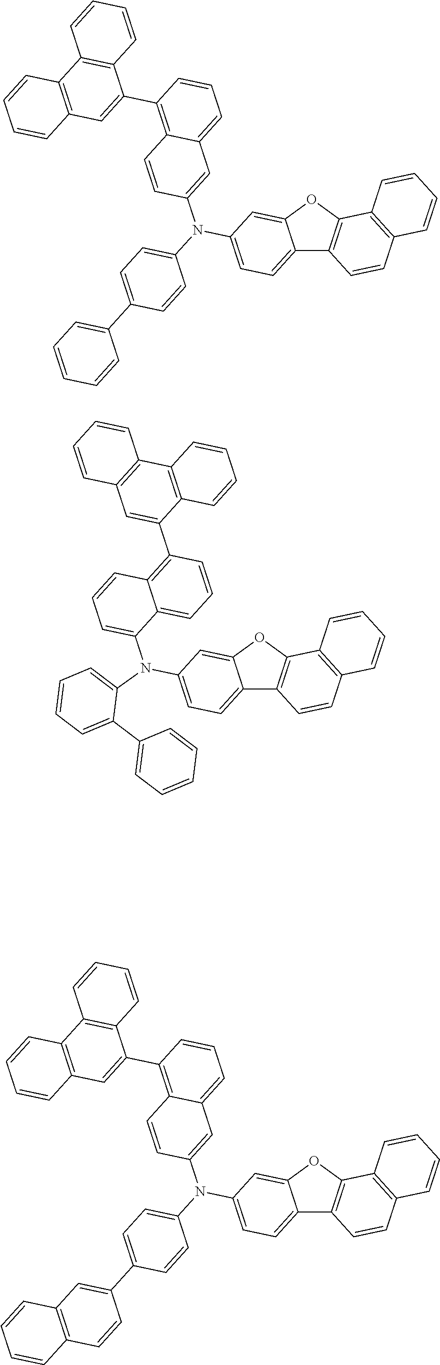

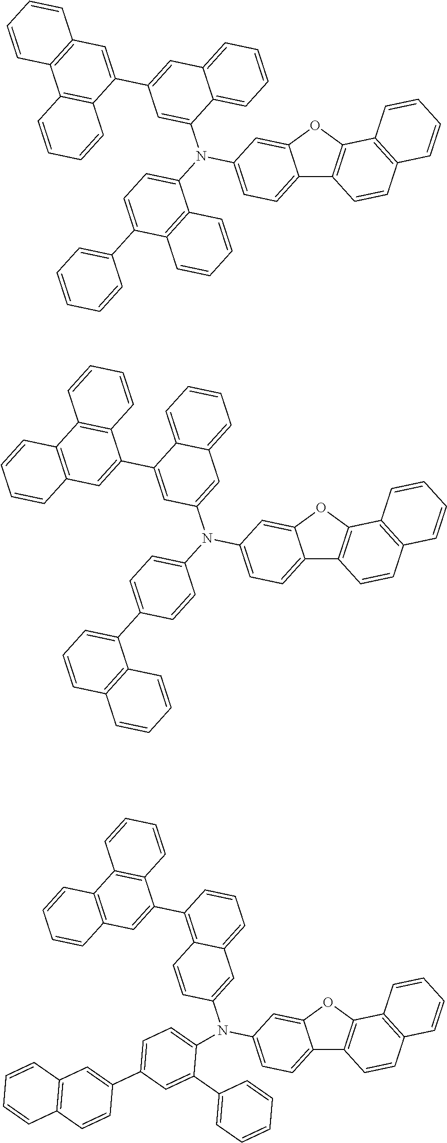 [SMILES: c1ccc(-c2ccc(N(c3ccc4c(c3)oc3c5ccccc5ccc43)c3cc(-c4cc5ccccc5c5ccccc45)cc4ccccc34)c3ccccc23)cc1 ; c1ccc2c(-c3ccc(N(c4cc(-c5cc6ccccc6c6ccccc56)c5ccccc5c4)c4ccc5c(c4)oc4c6ccccc6ccc54)cc3)cccc2c1 ; c1ccc(-c2cc(-c3ccc4ccccc4c3)ccc2N(c2ccc3c(-c4cc5ccccc5c5ccccc45)cccc3c2)c2ccc3c(c2)oc2c4ccccc4ccc32)cc1]

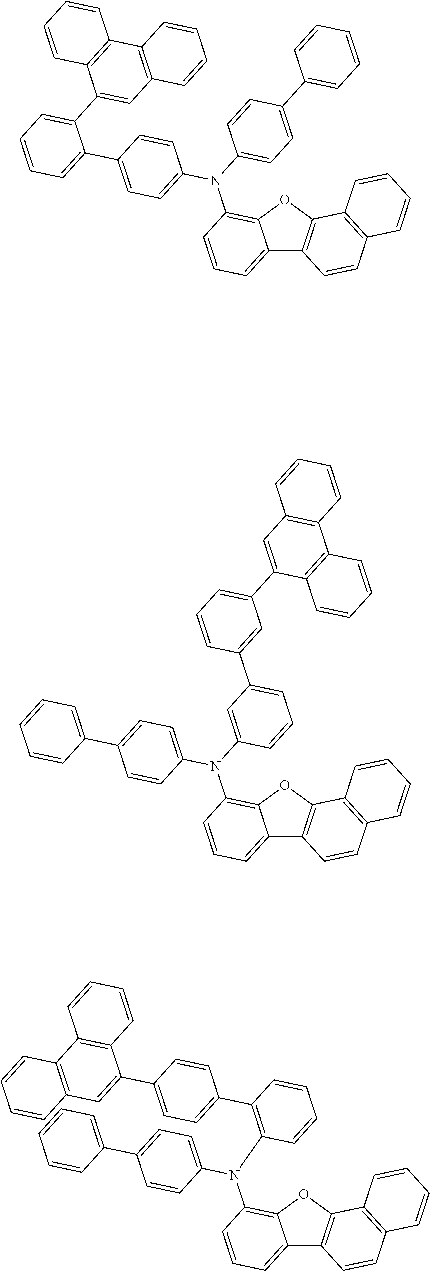 [SMILES: c1ccc(-c2ccc(N(c3ccc(-c4ccccc4-c4cc5ccccc5c5ccccc45)cc3)c3cccc4c3oc3c5ccccc5ccc43)cc2)cc1 ; c1ccc(-c2ccc(N(c3cccc(-c4cccc(-c5cc6ccccc6c6ccccc56)c4)c3)c3cccc4c3oc3c5ccccc5ccc43)cc2)cc1 ; c1ccc(-c2ccc(N(c3ccccc3-c3ccc(-c4cc5ccccc5c5ccccc45)cc3)c3cccc4c3oc3c5ccccc5ccc43)cc2)cc1]

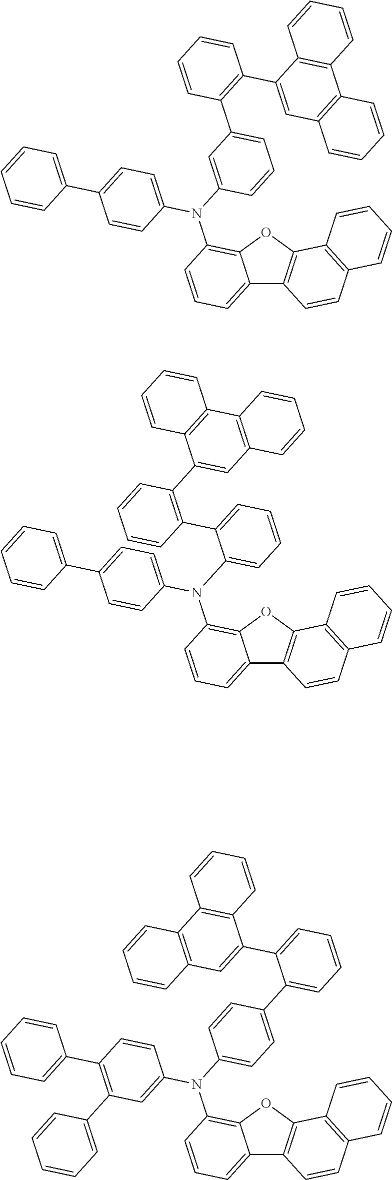 [SMILES: c1ccc(-c2ccc(N(c3cccc(-c4ccccc4-c4cc5ccccc5c5ccccc45)c3)c3cccc4c3oc3c5ccccc5ccc43)cc2)cc1 ; c1ccc(-c2ccc(N(c3ccccc3-c3ccccc3-c3cc4ccccc4c4ccccc34)c3cccc4c3oc3c5ccccc5ccc43)cc2)cc1 ; c1ccc(-c2ccc(N(c3ccc(-c4ccccc4-c4cc5ccccc5c5ccccc45)cc3)c3cccc4c3oc3c5ccccc5ccc43)cc2-c2ccccc2)cc1]

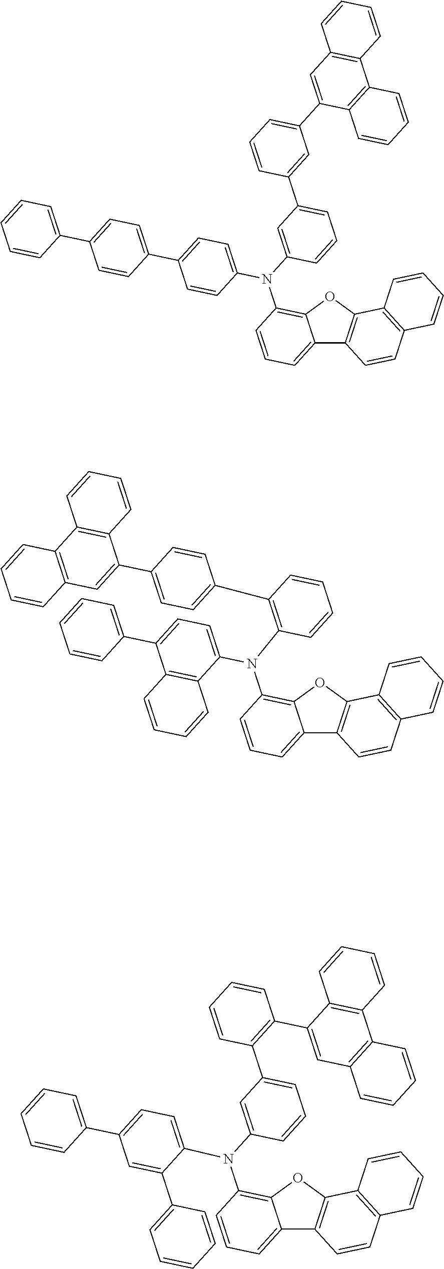 [SMILES: c1ccc(-c2ccc(-c3ccc(N(c4cccc(-c5cccc(-c6cc7ccccc7c7ccccc67)c5)c4)c4cccc5c4oc4c6ccccc6ccc54)cc3)cc2)cc1 ; c1ccc(-c2ccc(N(c3ccccc3-c3ccc(-c4cc5ccccc5c5ccccc45)cc3)c3cccc4c3oc3c5ccccc5ccc43)c3ccccc23)cc1 ; c1ccc(-c2ccc(N(c3cccc(-c4ccccc4-c4cc5ccccc5c5ccccc45)c3)c3cccc4c3oc3c5ccccc5ccc43)c(-c3ccccc3)c2)cc1]

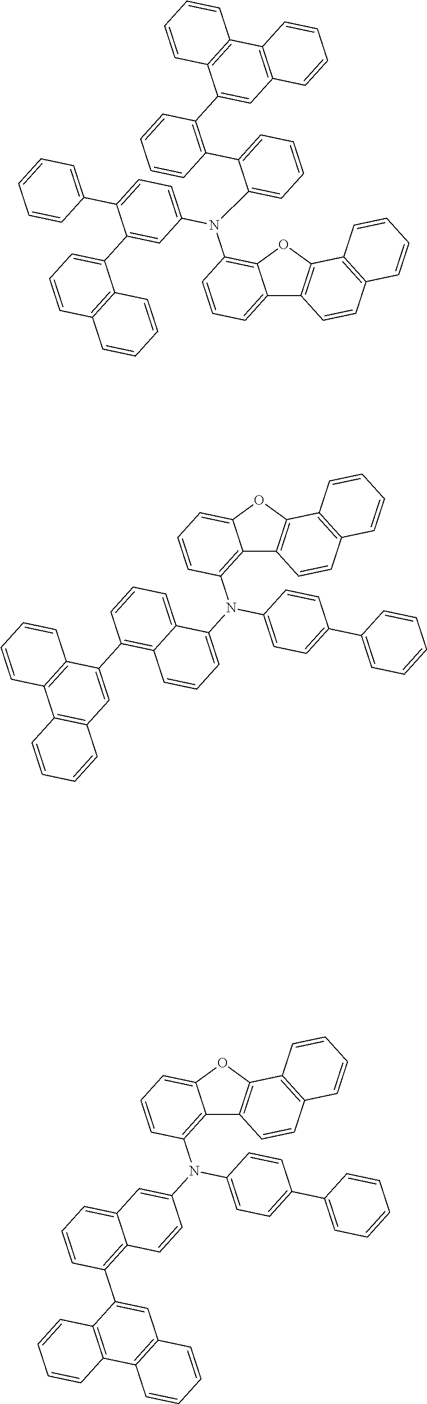 [SMILES: c1ccc(-c2ccc(N(c3ccccc3-c3ccccc3-c3cc4ccccc4c4ccccc34)c3cccc4c3oc3c5ccccc5ccc43)cc2-c2cccc3ccccc23)cc1 ; c1ccc(-c2ccc(N(c3cccc4c(-c5cc6ccccc6c6ccccc56)cccc34)c3cccc4oc5c6ccccc6ccc5c34)cc2)cc1 ; c1ccc(-c2ccc(N(c3ccc4c(-c5cc6ccccc6c6ccccc56)cccc4c3)c3cccc4oc5c6ccccc6ccc5c34)cc2)cc1]

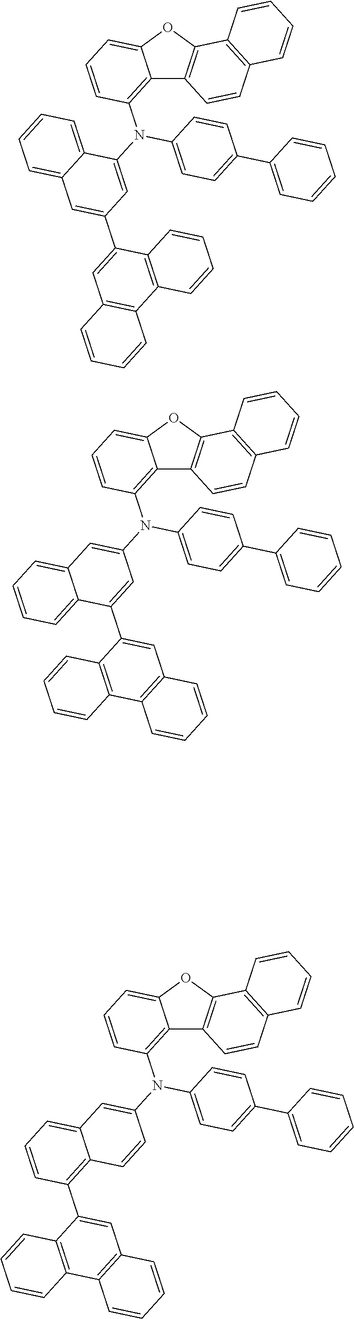 [SMILES: c1ccc(-c2ccc(N(c3cc(-c4cc5ccccc5c5ccccc45)cc4ccccc34)c3cccc4oc5c6ccccc6ccc5c34)cc2)cc1 ; c1ccc(-c2ccc(N(c3cc(-c4cc5ccccc5c5ccccc45)c4ccccc4c3)c3cccc4oc5c6ccccc6ccc5c34)cc2)cc1 ; c1ccc(-c2ccc(N(c3ccc4c(-c5cc6ccccc6c6ccccc56)cccc4c3)c3cccc4oc5c6ccccc6ccc5c34)cc2)cc1]

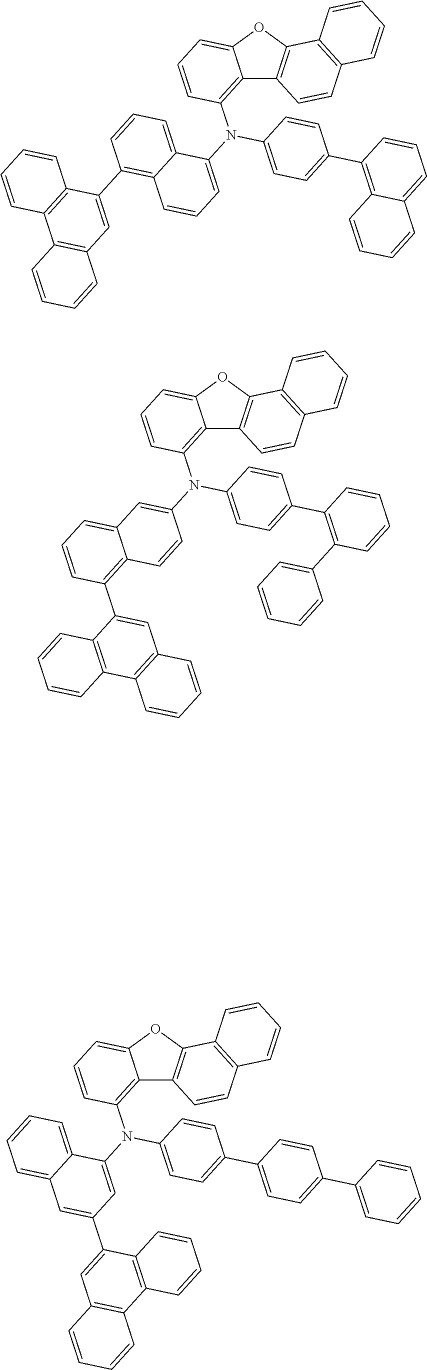 [SMILES: c1ccc2c(-c3ccc(N(c4cccc5c(-c6cc7ccccc7c7ccccc67)cccc45)c4cccc5oc6c7ccccc7ccc6c45)cc3)cccc2c1 ; c1ccc(-c2ccccc2-c2ccc(N(c3ccc4c(-c5cc6ccccc6c6ccccc56)cccc4c3)c3cccc4oc5c6ccccc6ccc5c34)cc2)cc1 ; c1ccc(-c2ccc(-c3ccc(N(c4cc(-c5cc6ccccc6c6ccccc56)cc5ccccc45)c4cccc5oc6c7ccccc7ccc6c45)cc3)cc2)cc1]

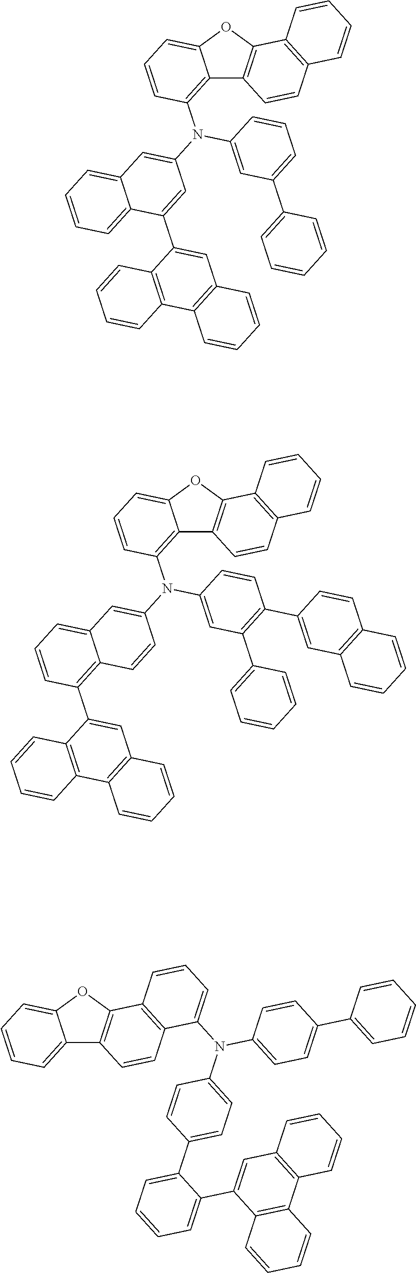 [SMILES: c1ccc(-c2cccc(N(c3cc(-c4cc5ccccc5c5ccccc45)c4ccccc4c3)c3cccc4oc5c6ccccc6ccc5c34)c2)cc1 ; c1ccc(-c2cc(N(c3ccc4c(-c5cc6ccccc6c6ccccc56)cccc4c3)c3cccc4oc5c6ccccc6ccc5c34)ccc2-c2ccc3ccccc3c2)cc1 ; c1ccc(-c2ccc(N(c3ccc(-c4ccccc4-c4cc5ccccc5c5ccccc45)cc3)c3cccc4c3ccc3c5ccccc5oc43)cc2)cc1]

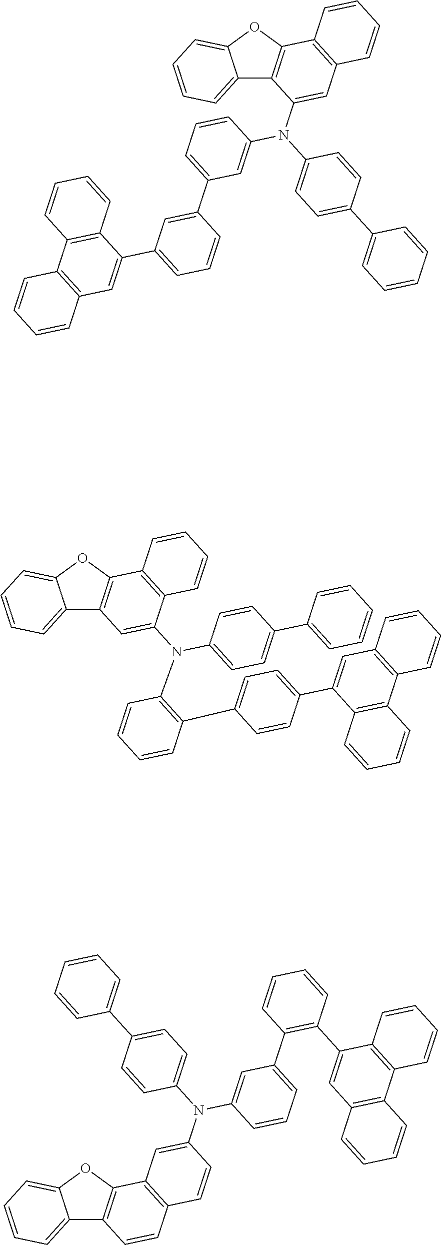 [SMILES: c1ccc(-c2ccc(N(c3cccc(-c4cccc(-c5cc6ccccc6c6ccccc56)c4)c3)c3cc4ccccc4c4oc5ccccc5c34)cc2)cc1 ; c1ccc(-c2ccc(N(c3ccccc3-c3ccc(-c4cc5ccccc5c5ccccc45)cc3)c3cc4c5ccccc5oc4c4ccccc34)cc2)cc1 ; c1ccc(-c2ccc(N(c3cccc(-c4ccccc4-c4cc5ccccc5c5ccccc45)c3)c3ccc4ccc5c6ccccc6oc5c4c3)cc2)cc1]

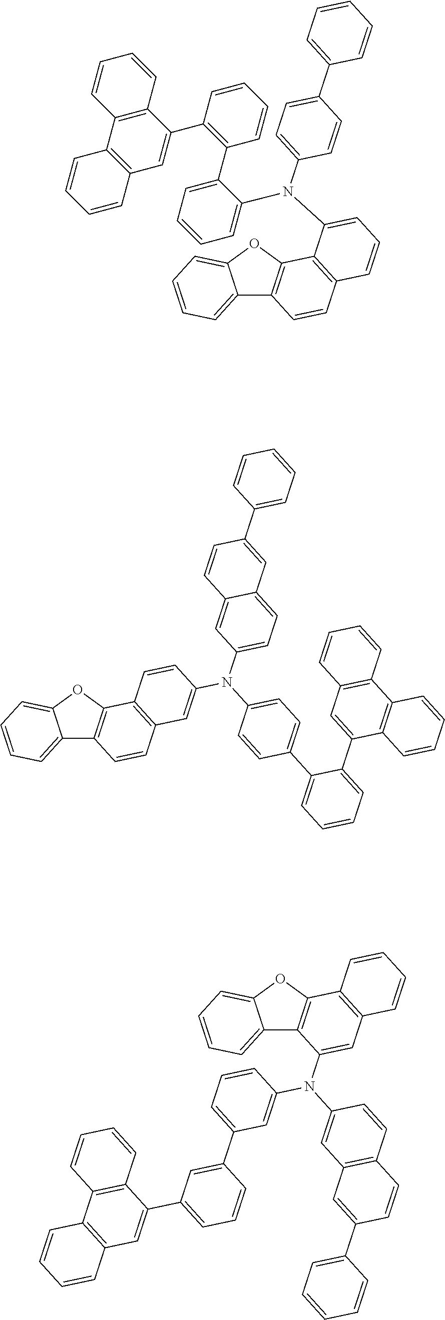 [SMILES: c1ccc(-c2ccc(N(c3ccccc3-c3ccccc3-c3cc4ccccc4c4ccccc34)c3cccc4ccc5c6ccccc6oc5c34)cc2)cc1 ; c1ccc(-c2ccc3cc(N(c4ccc(-c5ccccc5-c5cc6ccccc6c6ccccc56)cc4)c4ccc5c(ccc6c7ccccc7oc56)c4)ccc3c2)cc1 ; c1ccc(-c2ccc3ccc(N(c4cccc(-c5cccc(-c6cc7ccccc7c7ccccc67)c5)c4)c4cc5ccccc5c5oc6ccccc6c45)cc3c2)cc1]

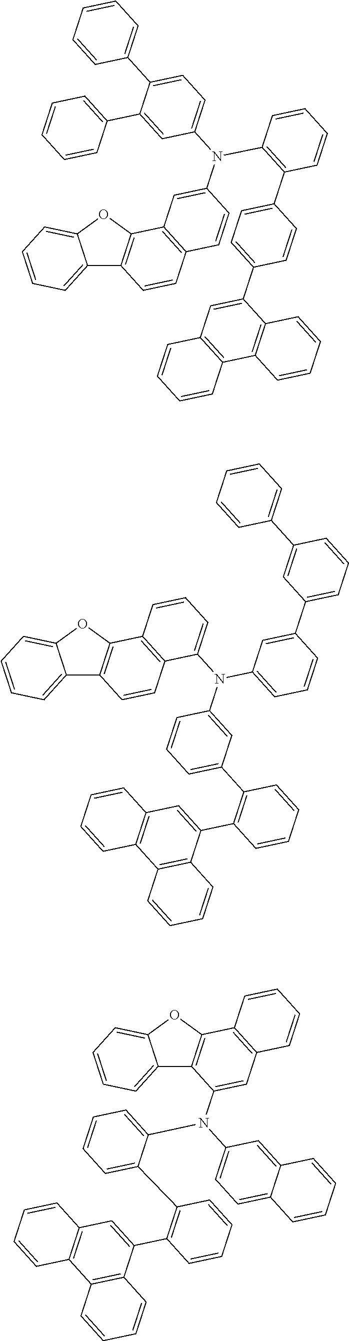 [SMILES: c1ccc(-c2ccc(N(c3ccc4ccc5c6ccccc6oc5c4c3)c3ccccc3-c3ccc(-c4cc5ccccc5c5ccccc45)cc3)cc2-c2ccccc2)cc1 ; c1ccc(-c2cccc(-c3cccc(N(c4cccc(-c5ccccc5-c5cc6ccccc6c6ccccc56)c4)c4cccc5c4ccc4c6ccccc6oc54)c3)c2)cc1 ; c1ccc(-c2cc3ccccc3c3ccccc23)c(-c2ccccc2N(c2ccc3ccccc3c2)c2cc3ccccc3c3oc4ccccc4c23)c1]

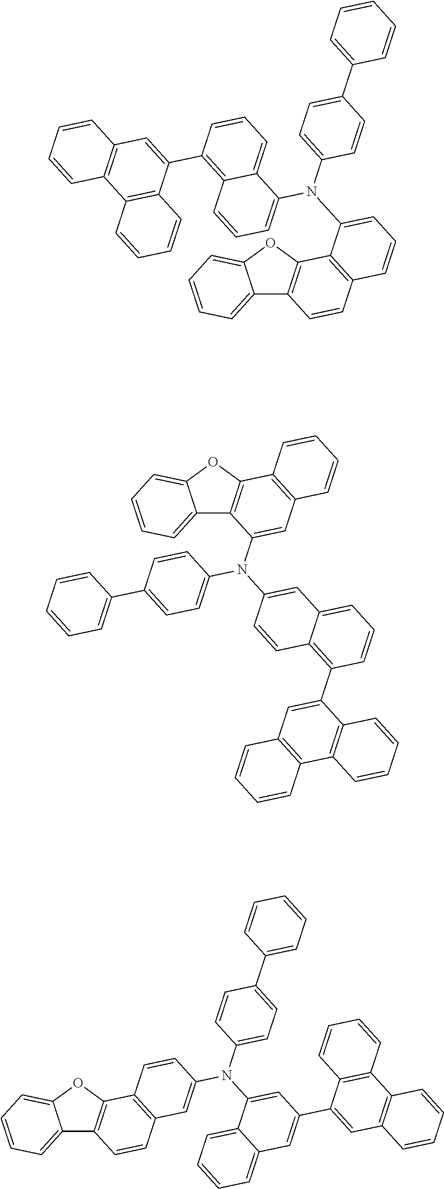 [SMILES: c1ccc(-c2ccc(N(c3cccc4c(-c5cc6ccccc6c6ccccc56)cccc34)c3cccc4ccc5c6ccccc6oc5c34)cc2)cc1 ; c1ccc(-c2ccc(N(c3ccc4c(-c5cc6ccccc6c6ccccc56)cccc4c3)c3cc4ccccc4c4oc5ccccc5c34)cc2)cc1 ; c1ccc(-c2ccc(N(c3ccc4c(ccc5c6ccccc6oc45)c3)c3cc(-c4cc5ccccc5c5ccccc45)cc4ccccc34)cc2)cc1]

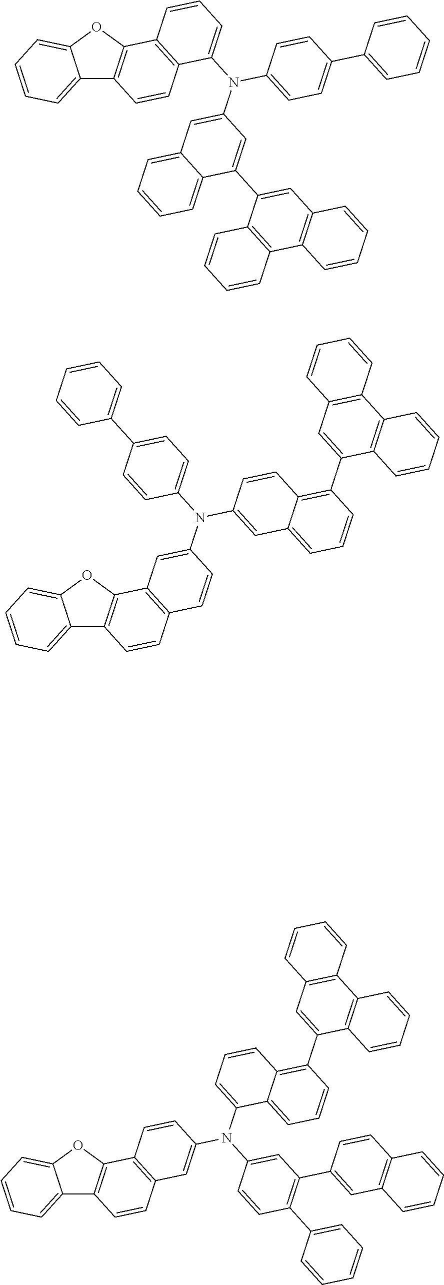 [SMILES: c1ccc(-c2ccc(N(c3cc(-c4cc5ccccc5c5ccccc45)c4ccccc4c3)c3cccc4c3ccc3c5ccccc5oc43)cc2)cc1 ; c1ccc(-c2ccc(N(c3ccc4c(-c5cc6ccccc6c6ccccc56)cccc4c3)c3ccc4ccc5c6ccccc6oc5c4c3)cc2)cc1 ; c1ccc(-c2ccc(N(c3ccc4c(ccc5c6ccccc6oc45)c3)c3cccc4c(-c5cc6ccccc6c6ccccc56)cccc34)cc2-c2ccc3ccccc3c2)cc1]

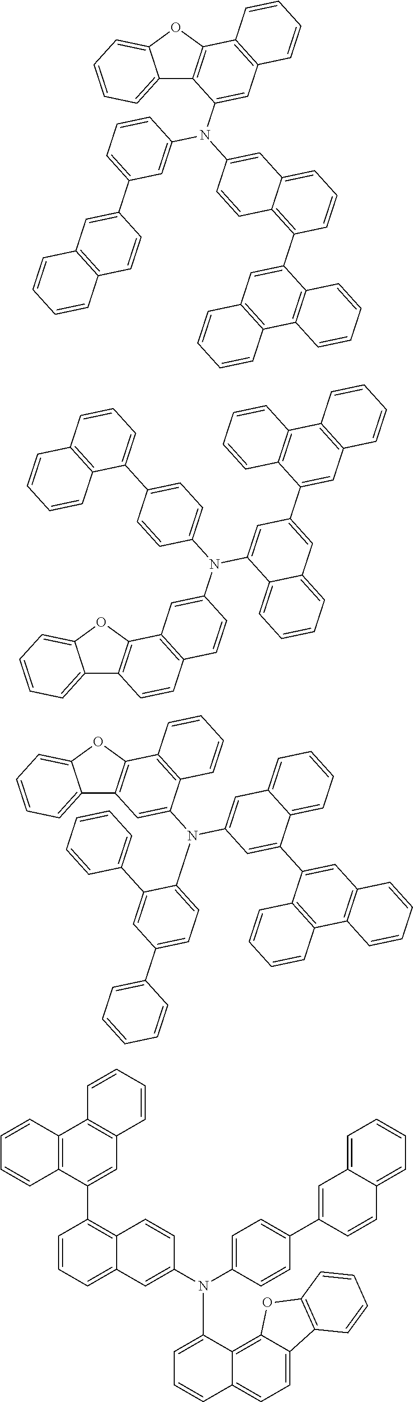

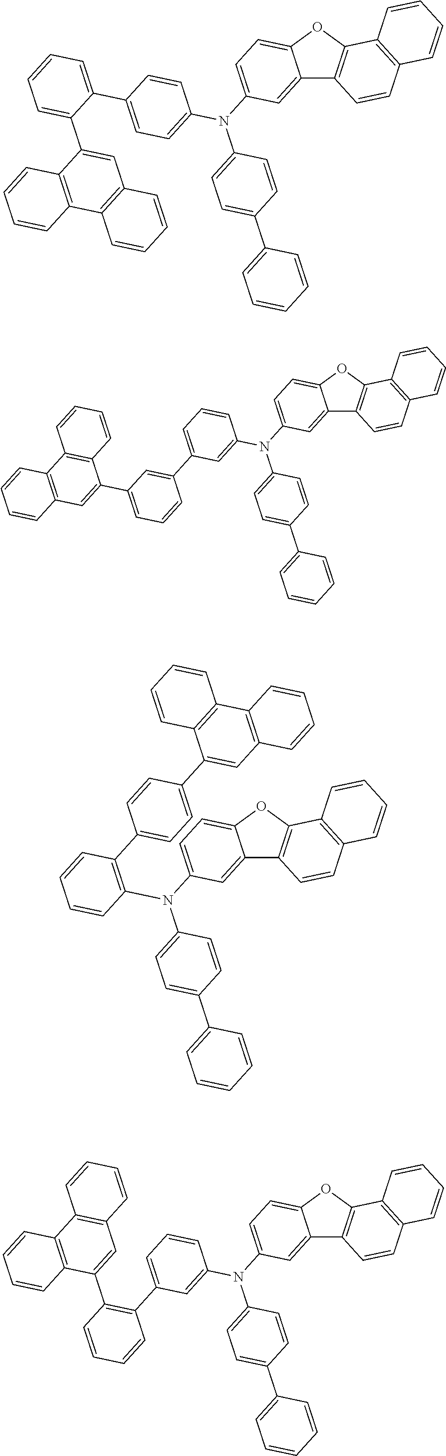 [SMILES: c1ccc(-c2ccc(N(c3ccc(-c4ccccc4-c4cc5ccccc5c5ccccc45)cc3)c3ccc4oc5c6ccccc6ccc5c4c3)cc2)cc1 ; c1ccc(-c2ccc(N(c3cccc(-c4cccc(-c5cc6ccccc6c6ccccc56)c4)c3)c3ccc4oc5c6ccccc6ccc5c4c3)cc2)cc1 ; c1ccc(-c2ccc(N(c3ccc4oc5c6ccccc6ccc5c4c3)c3ccccc3-c3ccc(-c4cc5ccccc5c5ccccc45)cc3)cc2)cc1 ; c1ccc(-c2ccc(N(c3cccc(-c4ccccc4-c4cc5ccccc5c5ccccc45)c3)c3ccc4oc5c6ccccc6ccc5c4c3)cc2)cc1]

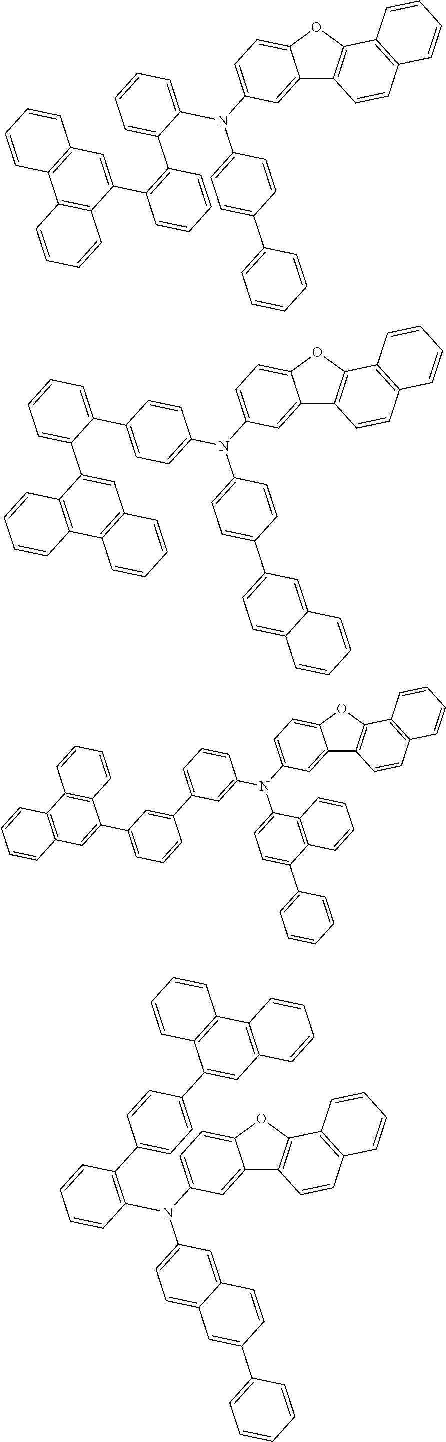

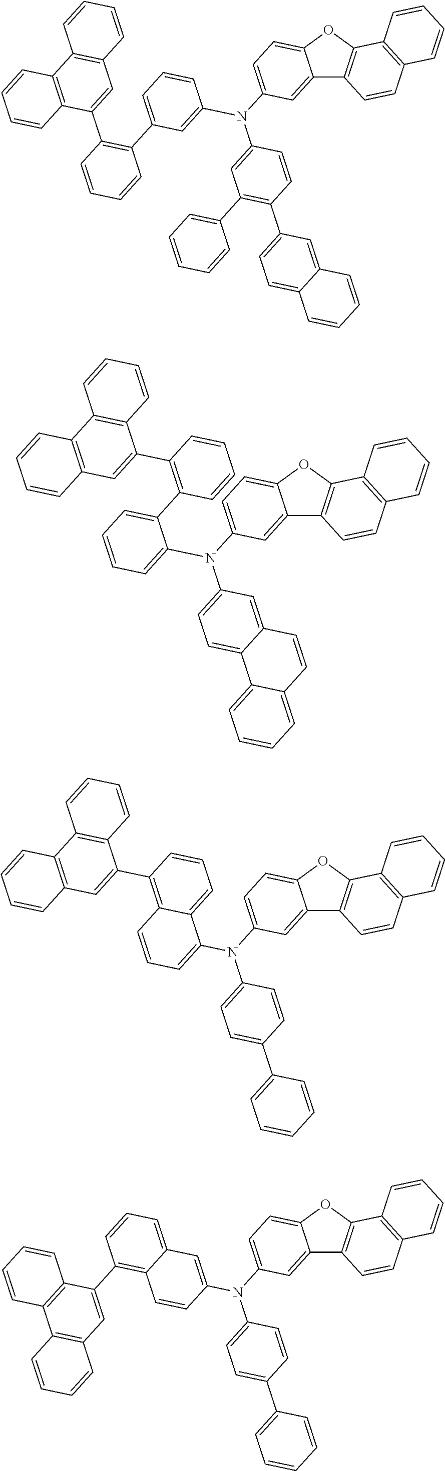 [SMILES: c1ccc(-c2cc(N(c3cccc(-c4ccccc4-c4cc5ccccc5c5ccccc45)c3)c3ccc4oc5c6ccccc6ccc5c4c3)ccc2-c2ccc3ccccc3c2)cc1 ; c1ccc(-c2cc3ccccc3c3ccccc23)c(-c2ccccc2N(c2ccc3c(ccc4ccccc43)c2)c2ccc3oc4c5ccccc5ccc4c3c2)c1 ; c1ccc(-c2ccc(N(c3ccc4oc5c6ccccc6ccc5c4c3)c3cccc4c(-c5cc6ccccc6c6ccccc56)cccc34)cc2)cc1 ; c1ccc(-c2ccc(N(c3ccc4c(-c5cc6ccccc6c6ccccc56)cccc4c3)c3ccc4oc5c6ccccc6ccc5c4c3)cc2)cc1]

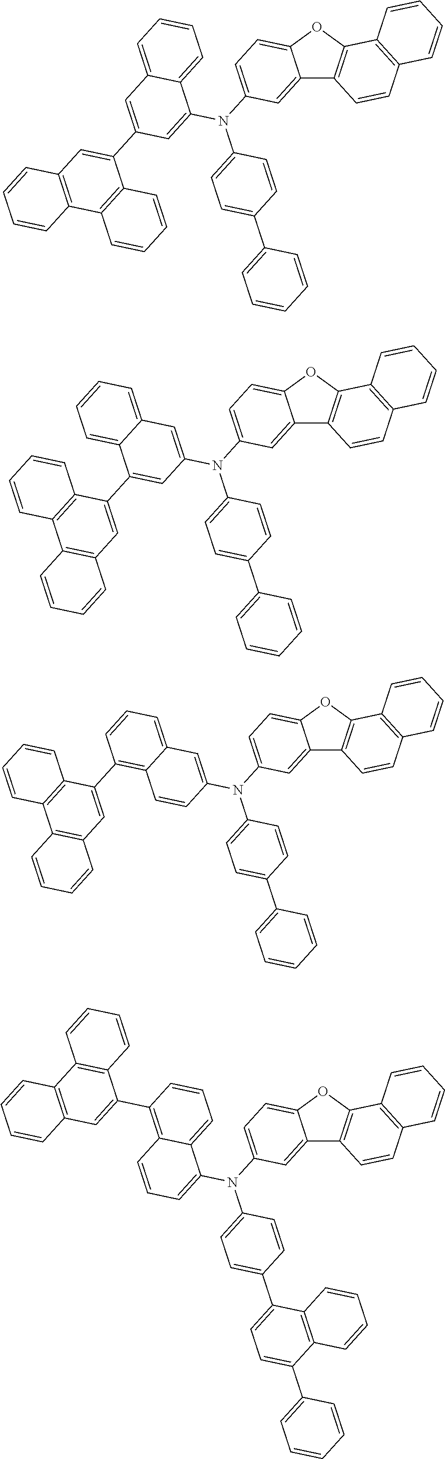 [SMILES: c1ccc(-c2ccc(N(c3ccc4oc5c6ccccc6ccc5c4c3)c3cc(-c4cc5ccccc5c5ccccc45)cc4ccccc34)cc2)cc1 ; c1ccc(-c2ccc(N(c3cc(-c4cc5ccccc5c5ccccc45)c4ccccc4c3)c3ccc4oc5c6ccccc6ccc5c4c3)cc2)cc1 ; c1ccc(-c2ccc(N(c3ccc4c(-c5cc6ccccc6c6ccccc56)cccc4c3)c3ccc4oc5c6ccccc6ccc5c4c3)cc2)cc1 ; c1ccc(-c2ccc(-c3ccc(N(c4ccc5oc6c7ccccc7ccc6c5c4)c4cccc5c(-c6cc7ccccc7c7ccccc67)cccc45)cc3)c3ccccc23)cc1]

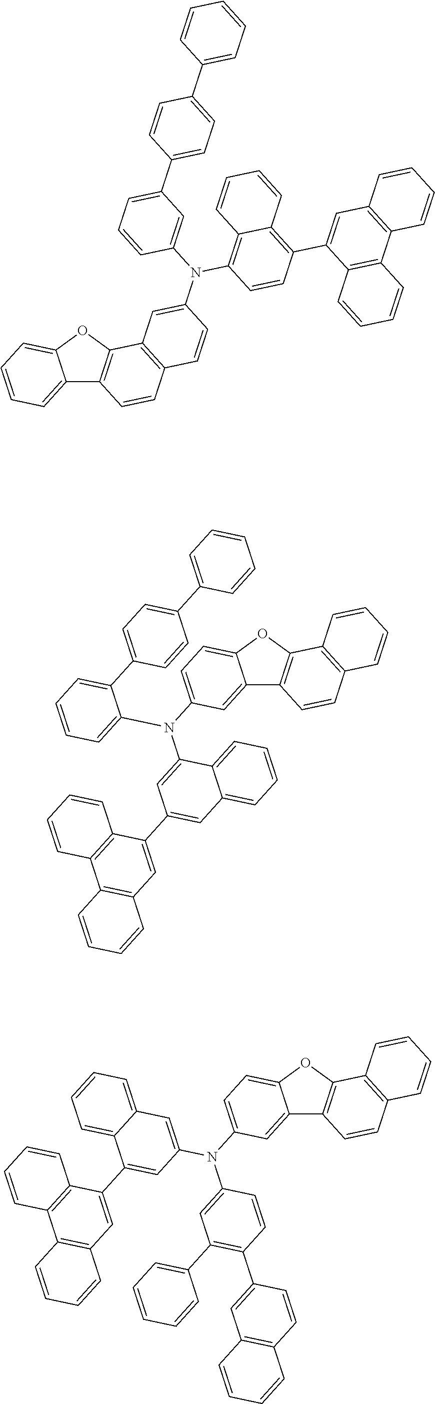 [SMILES: c1ccc(-c2ccc(-c3cccc(N(c4ccc5ccc6c7ccccc7oc6c5c4)c4ccc(-c5cc6ccccc6c6ccccc56)c5ccccc45)c3)cc2)cc1 ; c1ccc(-c2ccc(-c3ccccc3N(c3ccc4oc5c6ccccc6ccc5c4c3)c3cc(-c4cc5ccccc5c5ccccc45)cc4ccccc34)cc2)cc1 ; c1ccc(-c2cc(N(c3cc(-c4cc5ccccc5c5ccccc45)c4ccccc4c3)c3ccc4oc5c6ccccc6ccc5c4c3)ccc2-c2ccc3ccccc3c2)cc1]

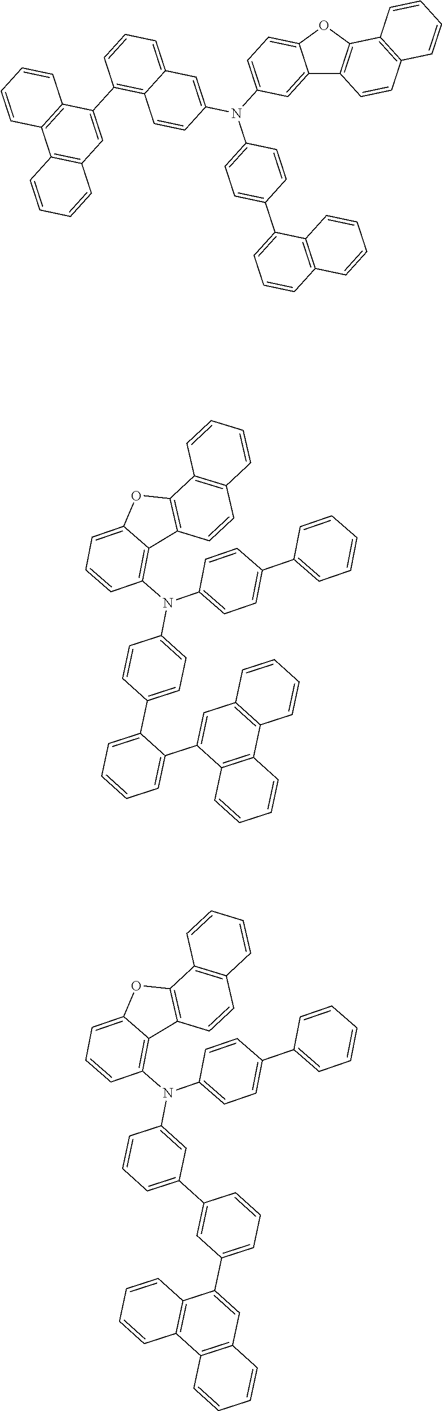 [SMILES: c1ccc2c(-c3ccc(N(c4ccc5c(-c6cc7ccccc7c7ccccc67)cccc5c4)c4ccc5oc6c7ccccc7ccc6c5c4)cc3)cccc2c1 ; c1ccc(-c2ccc(N(c3ccc(-c4ccccc4-c4cc5ccccc5c5ccccc45)cc3)c3cccc4oc5c6ccccc6ccc5c34)cc2)cc1 ; c1ccc(-c2ccc(N(c3cccc(-c4cccc(-c5cc6ccccc6c6ccccc56)c4)c3)c3cccc4oc5c6ccccc6ccc5c34)cc2)cc1]

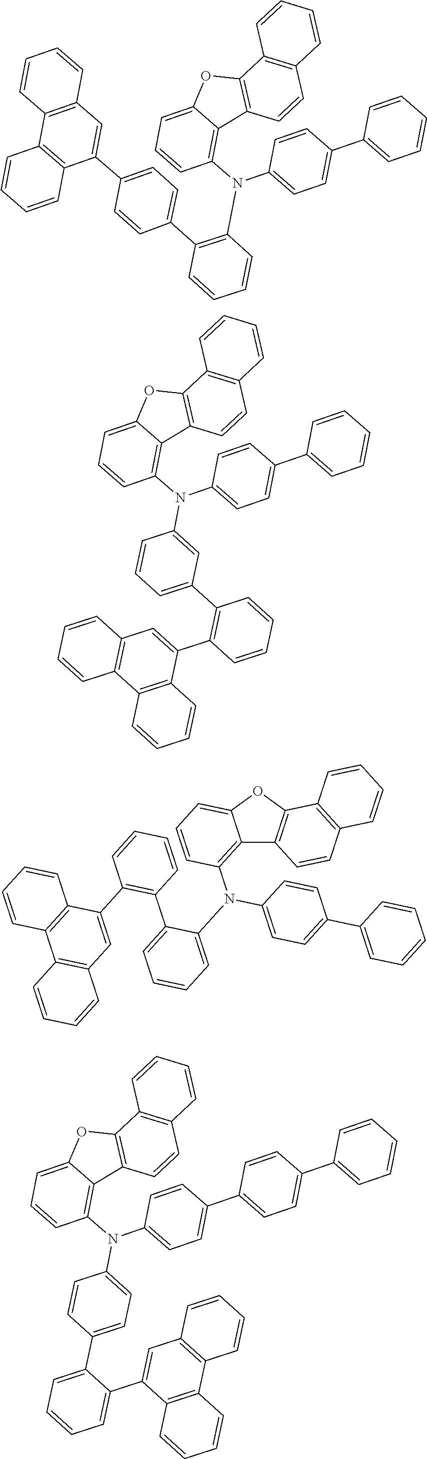

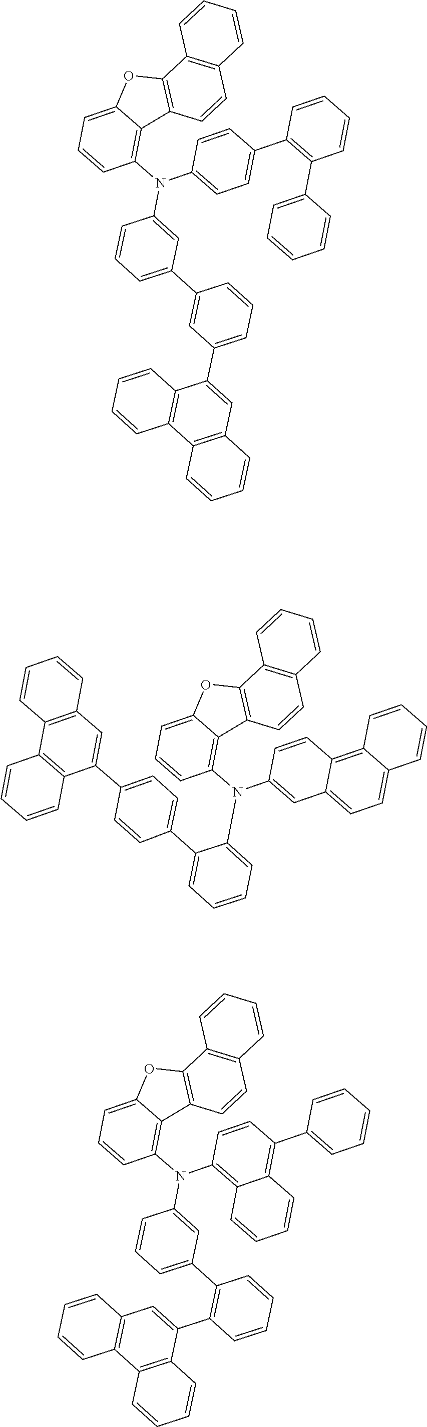 [SMILES: c1ccc(-c2ccccc2-c2ccc(N(c3cccc(-c4cccc(-c5cc6ccccc6c6ccccc56)c4)c3)c3cccc4oc5c6ccccc6ccc5c34)cc2)cc1 ; c1ccc(N(c2ccc3c(ccc4ccccc43)c2)c2cccc3oc4c5ccccc5ccc4c23)c(-c2ccc(-c3cc4ccccc4c4ccccc34)cc2)c1 ; c1ccc(-c2ccc(N(c3cccc(-c4ccccc4-c4cc5ccccc5c5ccccc45)c3)c3cccc4oc5c6ccccc6ccc5c34)c3ccccc23)cc1]

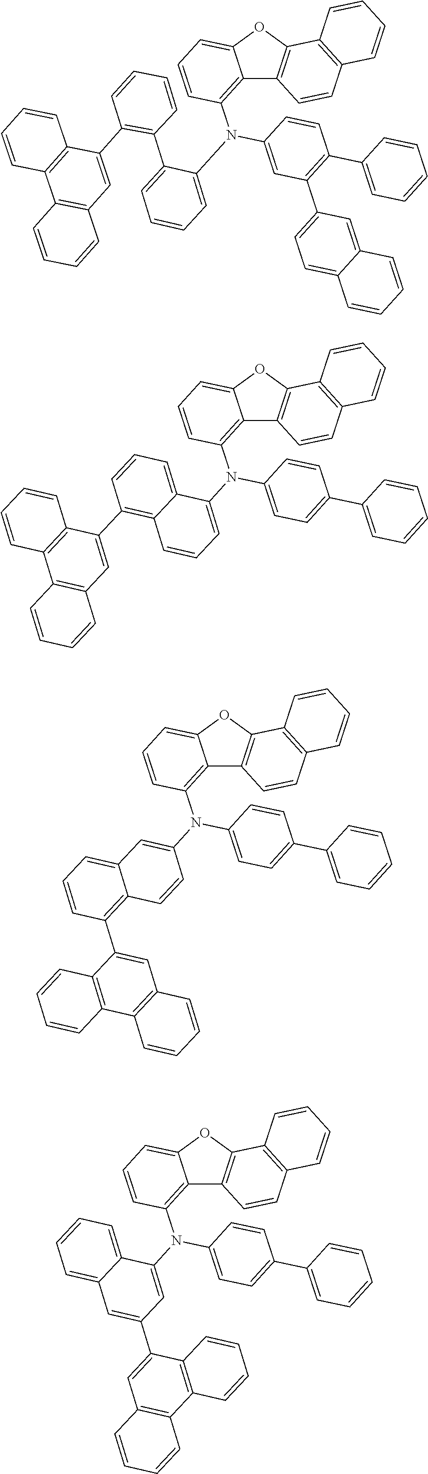

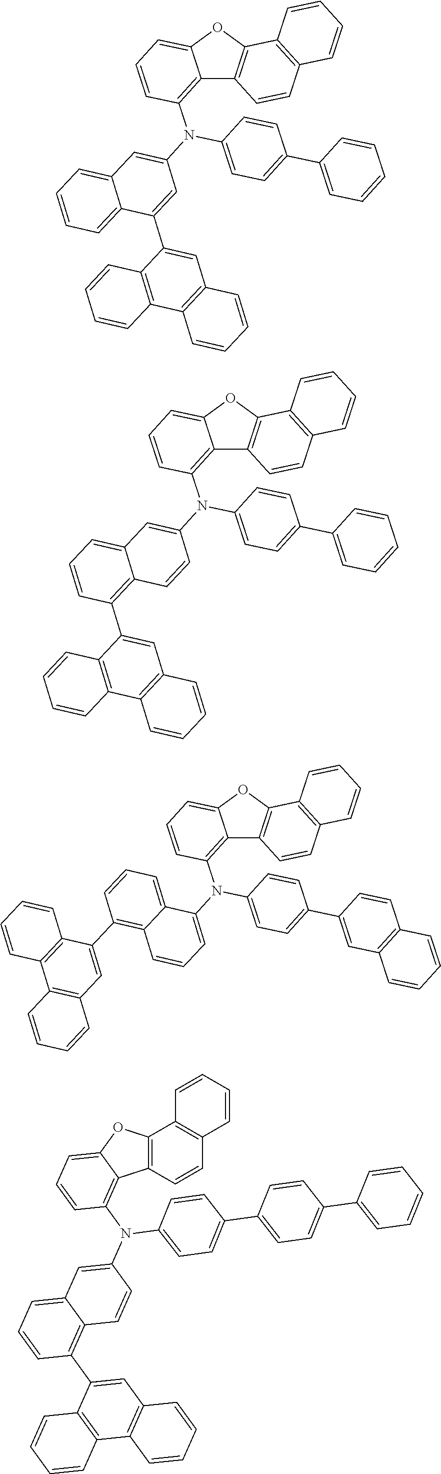 [SMILES: c1ccc(-c2ccc(N(c3cc(-c4cc5ccccc5c5ccccc45)c4ccccc4c3)c3cccc4oc5c6ccccc6ccc5c34)cc2)cc1 ; c1ccc(-c2ccc(N(c3ccc4c(-c5cc6ccccc6c6ccccc56)cccc4c3)c3cccc4oc5c6ccccc6ccc5c34)cc2)cc1 ; c1ccc2cc(-c3ccc(N(c4cccc5c(-c6cc7ccccc7c7ccccc67)cccc45)c4cccc5oc6c7ccccc7ccc6c45)cc3)ccc2c1 ; c1ccc(-c2ccc(-c3ccc(N(c4ccc5c(-c6cc7ccccc7c7ccccc67)cccc5c4)c4cccc5oc6c7ccccc7ccc6c45)cc3)cc2)cc1]

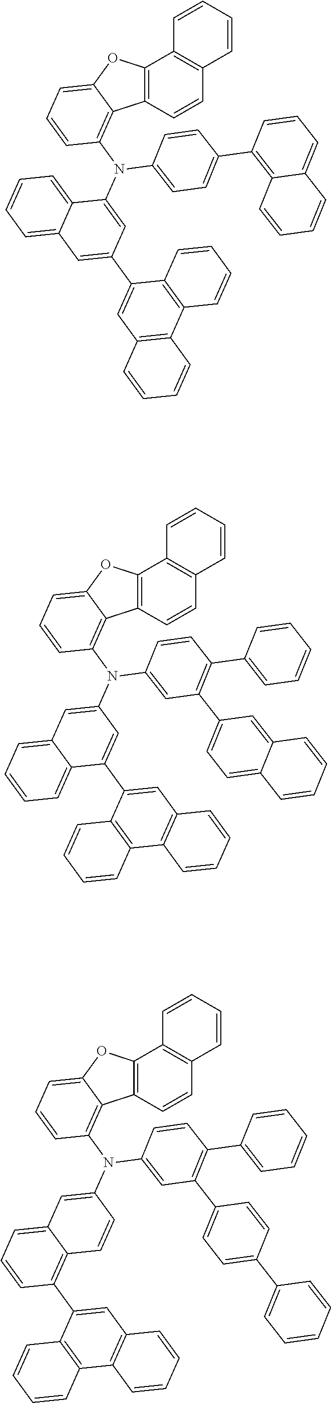 [SMILES: c1ccc2c(-c3ccc(N(c4cc(-c5cc6ccccc6c6ccccc56)cc5ccccc45)c4cccc5oc6c7ccccc7ccc6c45)cc3)cccc2c1 ; c1ccc(-c2ccc(N(c3cc(-c4cc5ccccc5c5ccccc45)c4ccccc4c3)c3cccc4oc5c6ccccc6ccc5c34)cc2-c2ccc3ccccc3c2)cc1 ; c1ccc(-c2ccc(-c3cc(N(c4ccc5c(-c6cc7ccccc7c7ccccc67)cccc5c4)c4cccc5oc6c7ccccc7ccc6c45)ccc3-c3ccccc3)cc2)cc1]

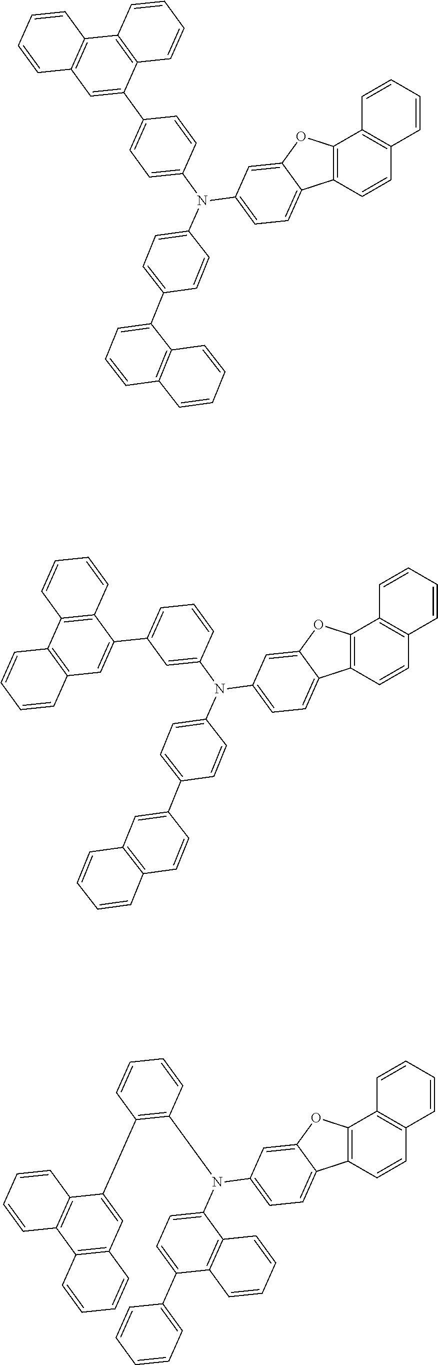 [SMILES: c1ccc2c(-c3ccc(N(c4ccc(-c5cc6ccccc6c6ccccc56)cc4)c4ccc5c(c4)oc4c6ccccc6ccc54)cc3)cccc2c1 ; c1cc(-c2cc3ccccc3c3ccccc23)cc(N(c2ccc(-c3ccc4ccccc4c3)cc2)c2ccc3c(c2)oc2c4ccccc4ccc32)c1 ; c1ccc(-c2ccc(N(c3ccc4c(c3)oc3c5ccccc5ccc43)c3ccccc3-c3cc4ccccc4c4ccccc34)c3ccccc23)cc1]

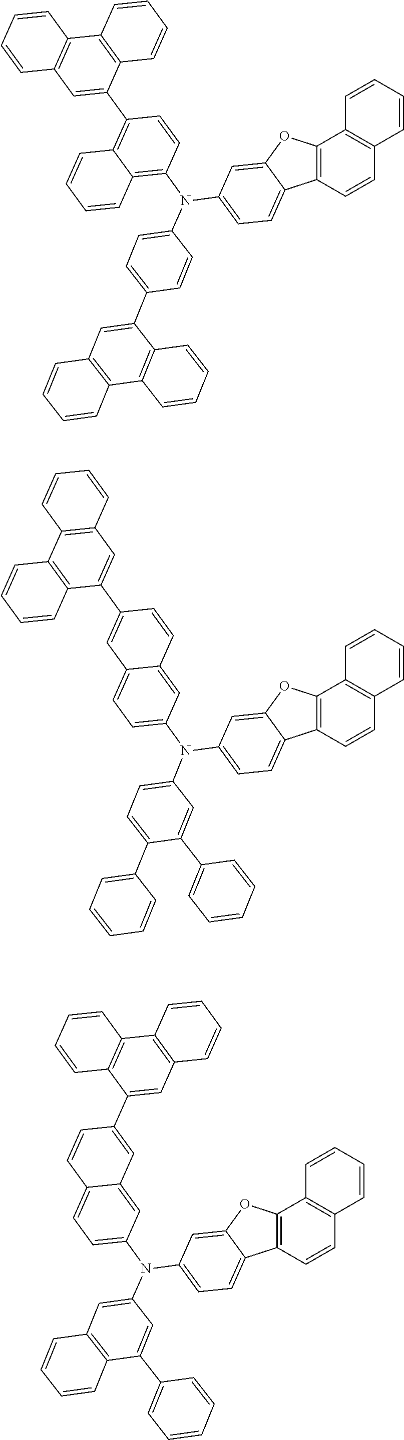 [SMILES: c1ccc2c(c1)cc(-c1ccc(N(c3ccc4c(c3)oc3c5ccccc5ccc43)c3ccc(-c4cc5ccccc5c5ccccc45)c4ccccc34)cc1)c1ccccc12 ; c1ccc(-c2ccc(N(c3ccc4cc(-c5cc6ccccc6c6ccccc56)ccc4c3)c3ccc4c(c3)oc3c5ccccc5ccc43)cc2-c2ccccc2)cc1 ; c1ccc(-c2cc(N(c3ccc4ccc(-c5cc6ccccc6c6ccccc56)cc4c3)c3ccc4c(c3)oc3c5ccccc5ccc43)cc3ccccc23)cc1]

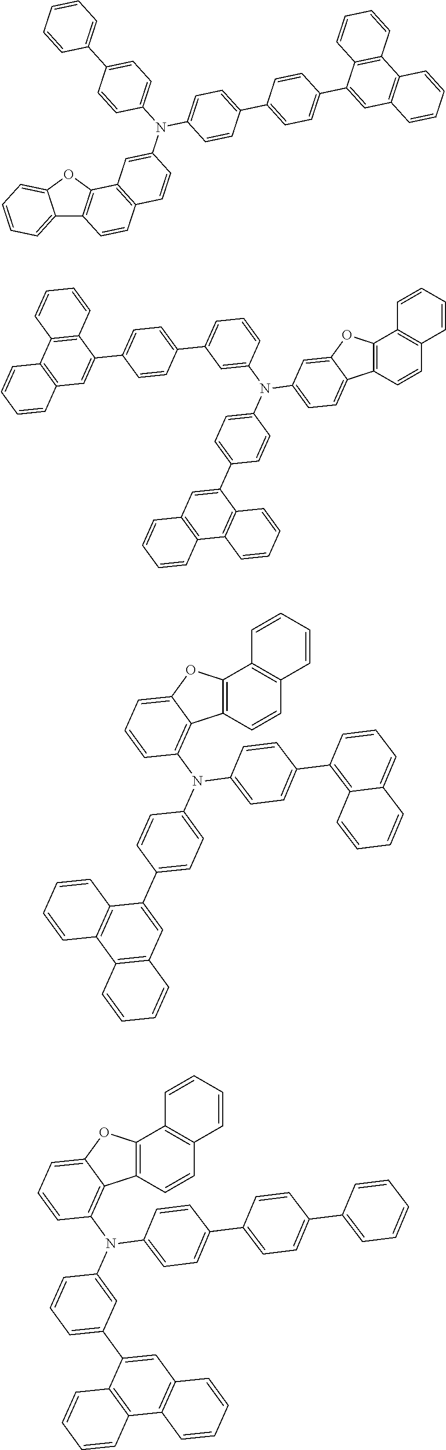 [SMILES: c1ccc(-c2ccc(N(c3ccc(-c4ccc(-c5cc6ccccc6c6ccccc56)cc4)cc3)c3ccc4ccc5c6ccccc6oc5c4c3)cc2)cc1 ; c1cc(-c2ccc(-c3cc4ccccc4c4ccccc34)cc2)cc(N(c2ccc(-c3cc4ccccc4c4ccccc34)cc2)c2ccc3c(c2)oc2c4ccccc4ccc32)c1 ; c1ccc2c(-c3ccc(N(c4ccc(-c5cc6ccccc6c6ccccc56)cc4)c4cccc5oc6c7ccccc7ccc6c45)cc3)cccc2c1 ; c1ccc(-c2ccc(-c3ccc(N(c4cccc(-c5cc6ccccc6c6ccccc56)c4)c4cccc5oc6c7ccccc7ccc6c45)cc3)cc2)cc1]

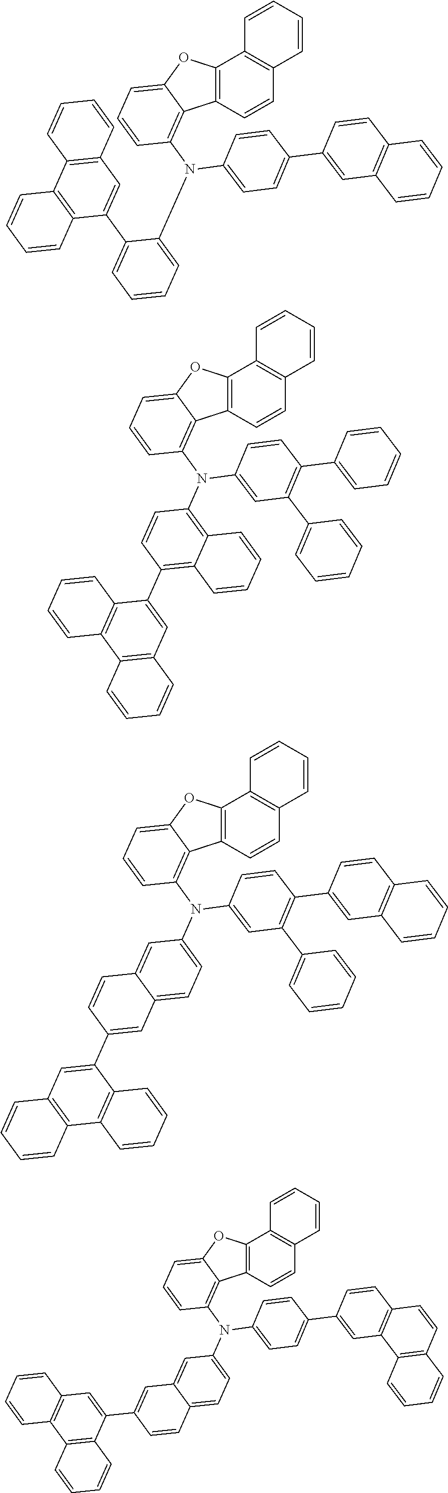 [SMILES: c1ccc(N(c2ccc(-c3ccc4ccccc4c3)cc2)c2cccc3oc4c5ccccc5ccc4c23)c(-c2cc3ccccc3c3ccccc23)c1 ; c1ccc(-c2ccc(N(c3ccc(-c4cc5ccccc5c5ccccc45)c4ccccc34)c3cccc4oc5c6ccccc6ccc5c34)cc2-c2ccccc2)cc1 ; c1ccc(-c2cc(N(c3ccc4cc(-c5cc6ccccc6c6ccccc56)ccc4c3)c3cccc4oc5c6ccccc6ccc5c34)ccc2-c2ccc3ccccc3c2)cc1 ; c1ccc2c(c1)ccc1ccc(-c3ccc(N(c4ccc5ccc(-c6cc7ccccc7c7ccccc67)cc5c4)c4cccc5oc6c7ccccc7ccc6c45)cc3)cc12]

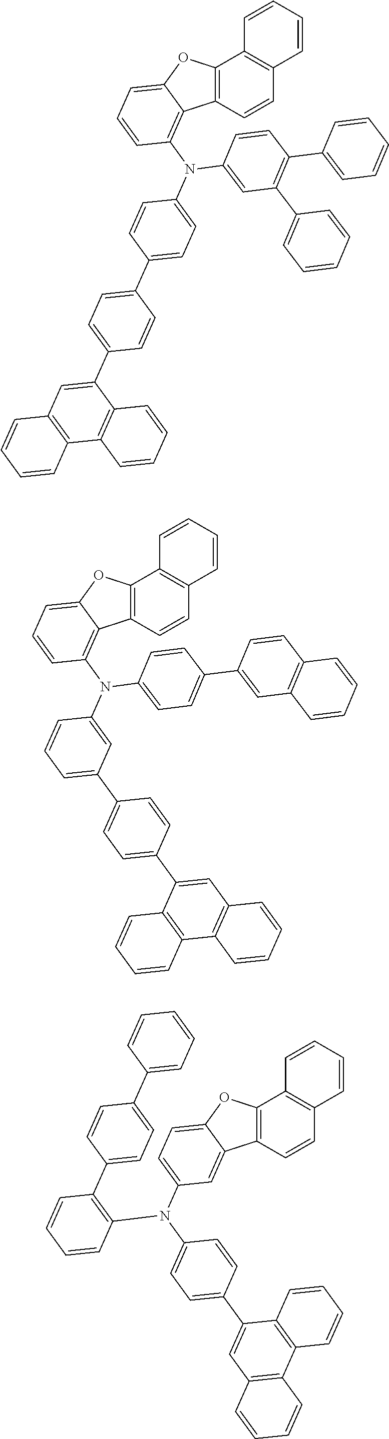

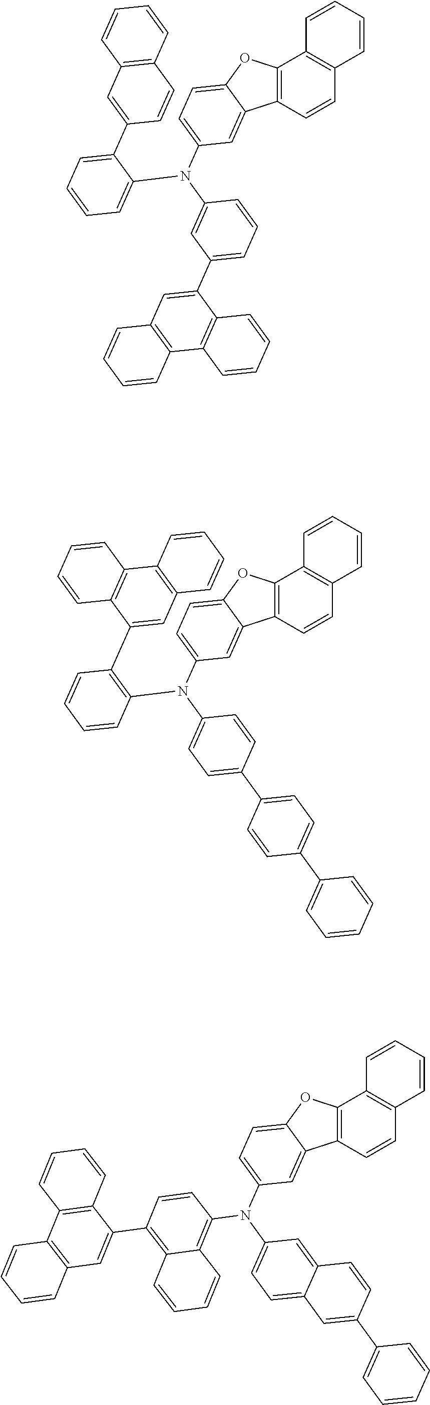 [SMILES: c1cc(-c2cc3ccccc3c3ccccc23)cc(N(c2ccc3oc4c5ccccc5ccc4c3c2)c2ccccc2-c2ccc3ccccc3c2)c1 ; c1ccc(-c2ccc(-c3ccc(N(c4ccc5oc6c7ccccc7ccc6c5c4)c4ccccc4-c4cc5ccccc5c5ccccc45)cc3)cc2)cc1 ; c1ccc(-c2ccc3cc(N(c4ccc5oc6c7ccccc7ccc6c5c4)c4ccc(-c5cc6ccccc6c6ccccc56)c5ccccc45)ccc3c2)cc1]

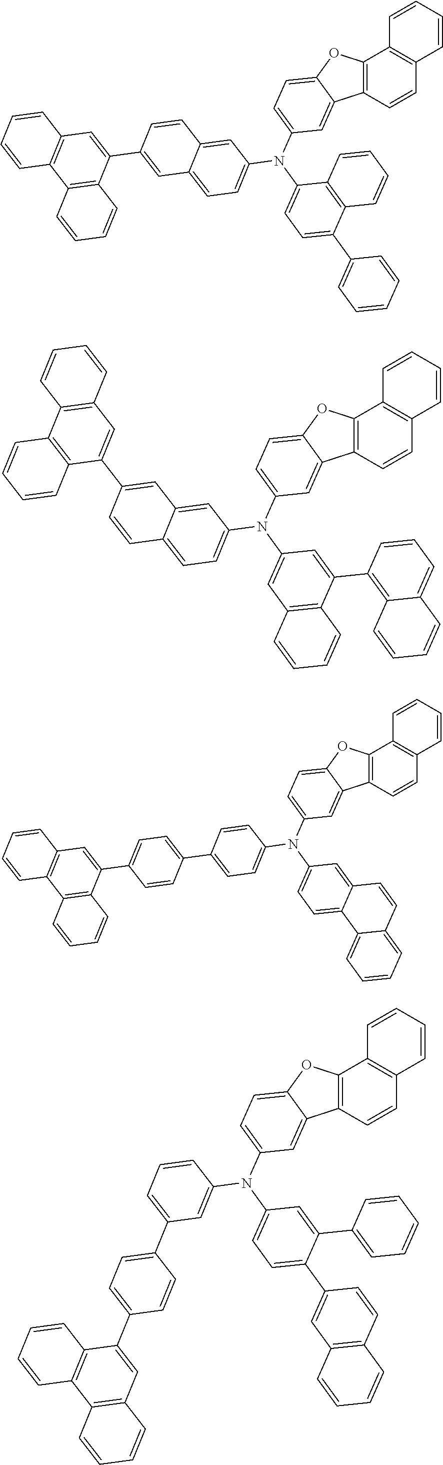

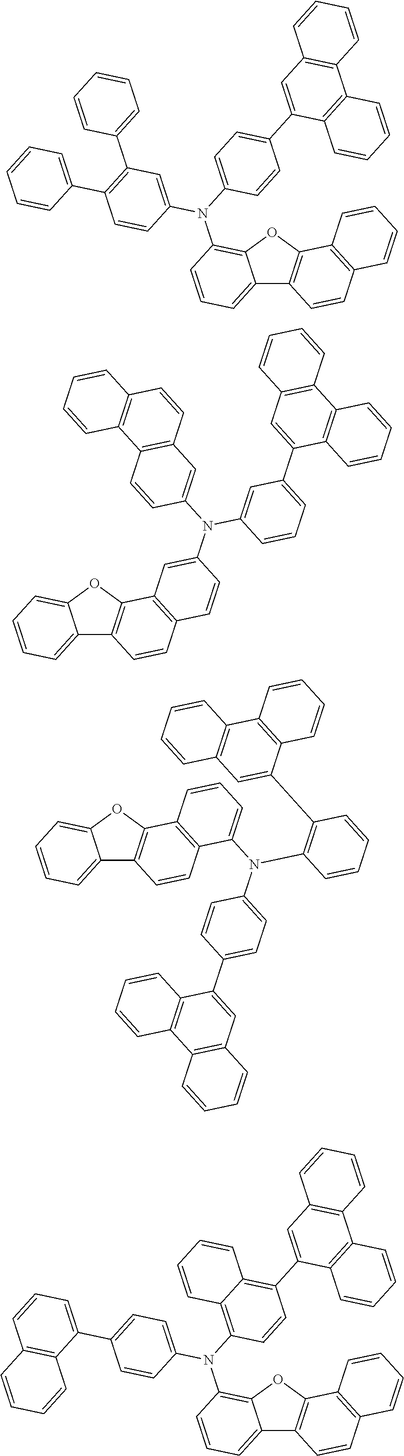

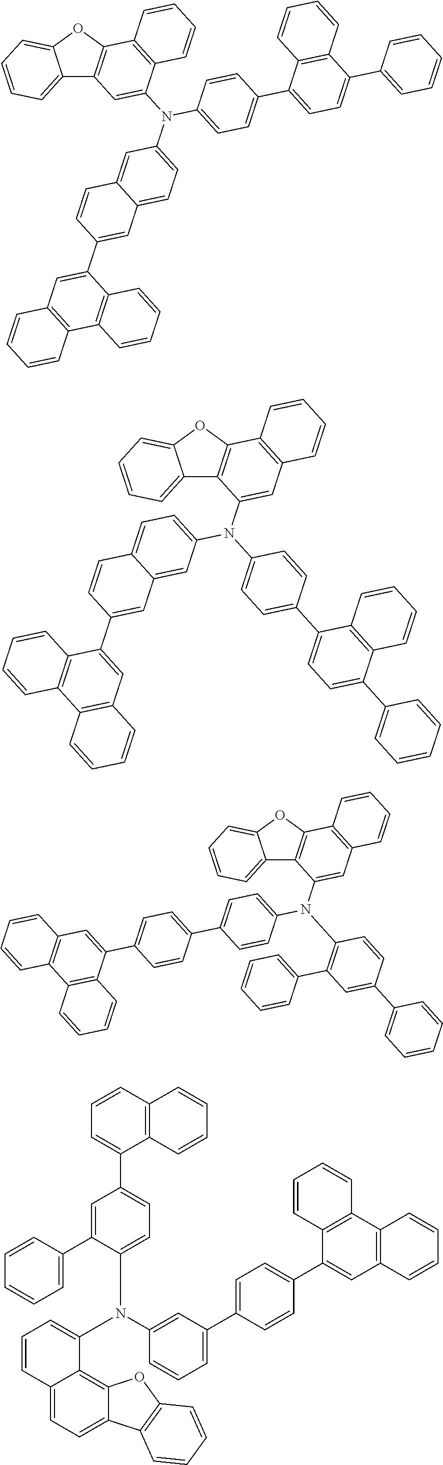

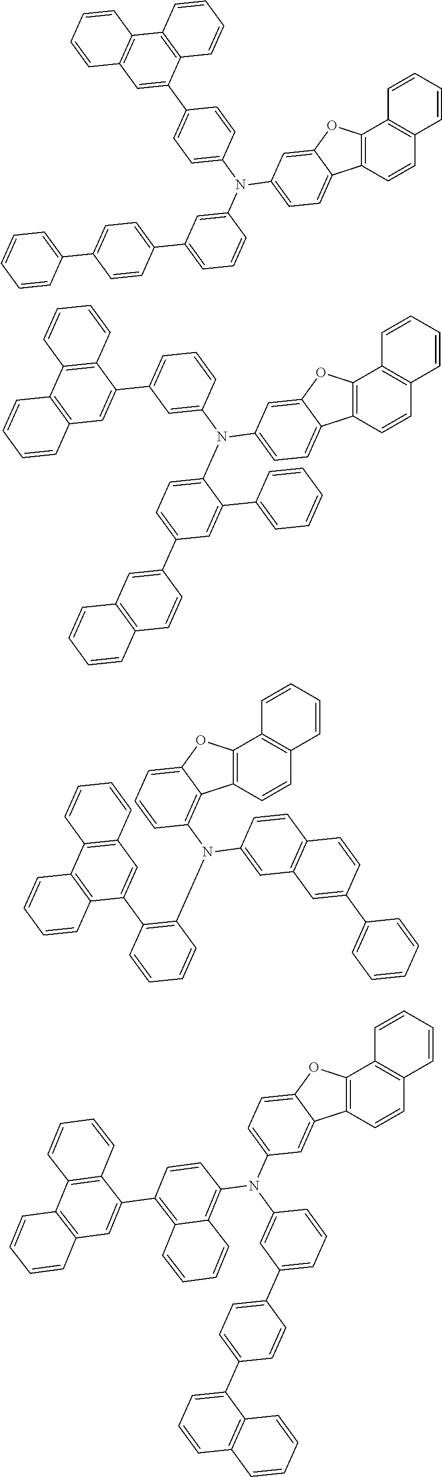

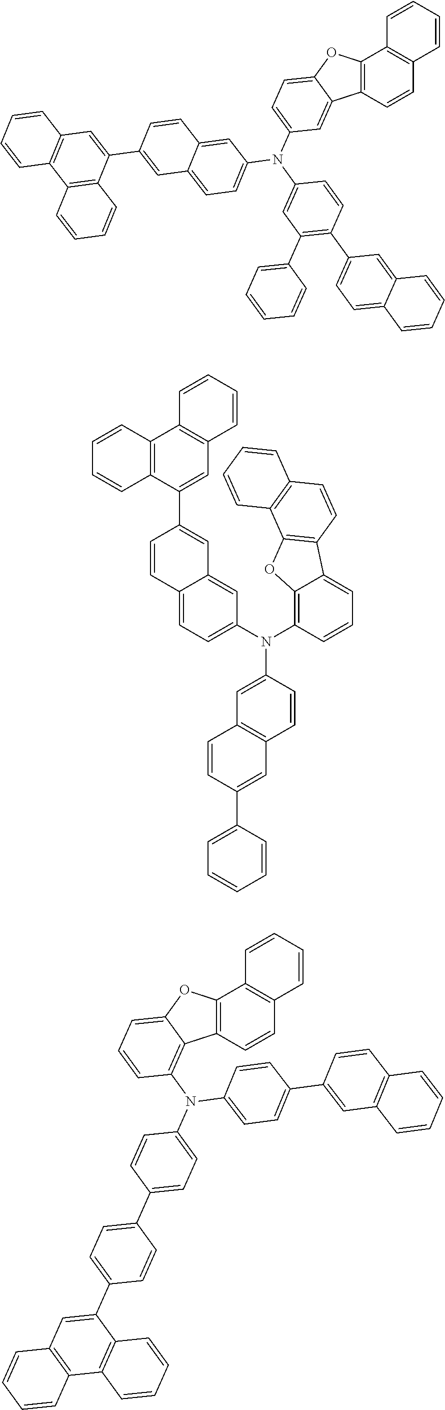 [SMILES: c1ccc(-c2cc(N(c3ccc4cc(-c5cc6ccccc6c6ccccc56)ccc4c3)c3ccc4oc5c6ccccc6ccc5c4c3)ccc2-c2ccc3ccccc3c2)cc1 ; c1ccc(-c2ccc3cc(N(c4ccc5ccc(-c6cc7ccccc7c7ccccc67)cc5c4)c4cccc5c4oc4c6ccccc6ccc54)ccc3c2)cc1 ; c1ccc2cc(-c3ccc(N(c4ccc(-c5ccc(-c6cc7ccccc7c7ccccc67)cc5)cc4)c4cccc5oc6c7ccccc7ccc6c45)cc3)ccc2c1]

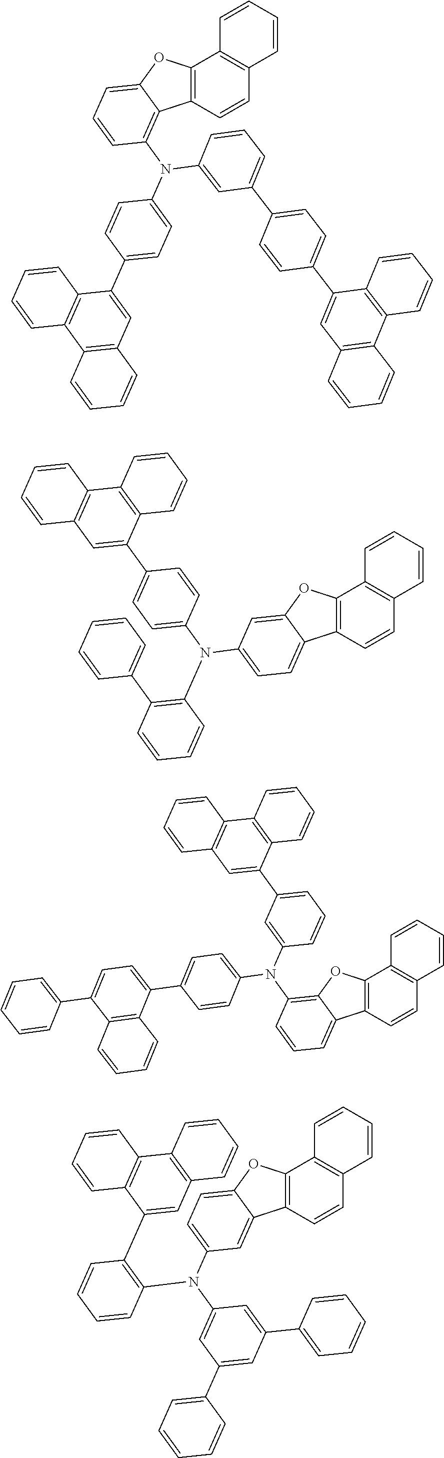

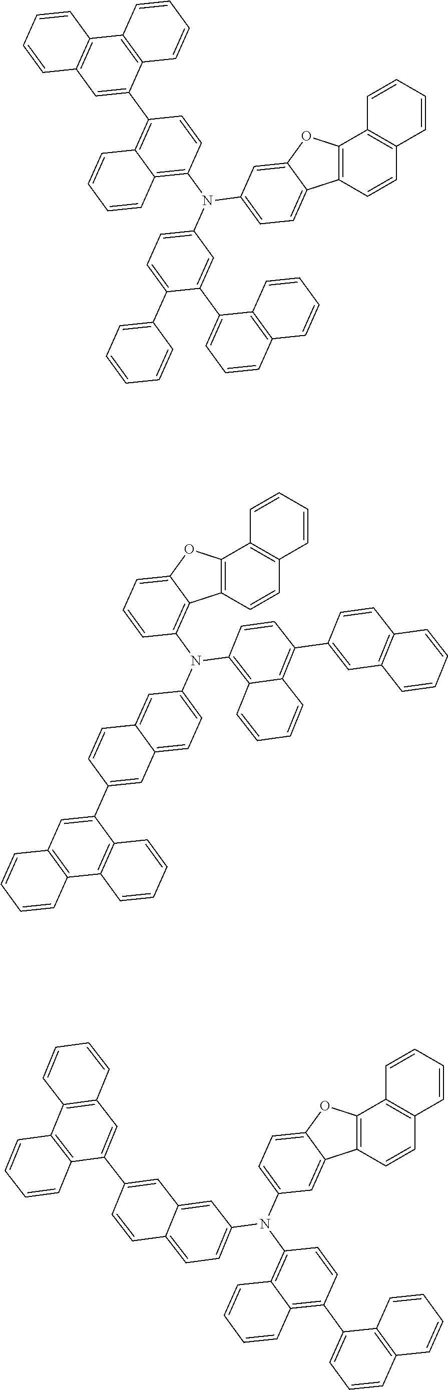 [SMILES: c1ccc(-c2ccc(N(c3ccc4c(c3)oc3c5ccccc5ccc43)c3ccc(-c4cc5ccccc5c5ccccc45)c4ccccc34)cc2-c2cccc3ccccc23)cc1 ; c1ccc2cc(-c3ccc(N(c4ccc5cc(-c6cc7ccccc7c7ccccc67)ccc5c4)c4cccc5oc6c7ccccc7ccc6c45)c4ccccc34)ccc2c1 ; c1ccc2c(-c3ccc(N(c4ccc5ccc(-c6cc7ccccc7c7ccccc67)cc5c4)c4ccc5oc6c7ccccc7ccc6c5c4)c4ccccc34)cccc2c1]

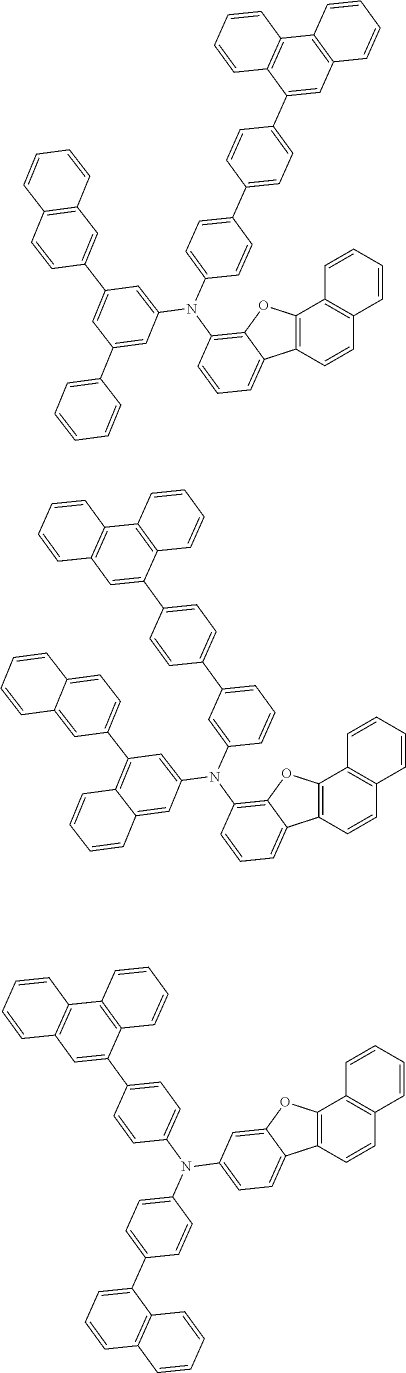 [SMILES: c1ccc(-c2cc(-c3ccc4ccccc4c3)cc(N(c3ccc(-c4ccc(-c5cc6ccccc6c6ccccc56)cc4)cc3)c3cccc4c3oc3c5ccccc5ccc43)c2)cc1 ; c1cc(-c2ccc(-c3cc4ccccc4c4ccccc34)cc2)cc(N(c2cc(-c3ccc4ccccc4c3)c3ccccc3c2)c2cccc3c2oc2c4ccccc4ccc32)c1 ; c1ccc2c(-c3ccc(N(c4ccc(-c5cc6ccccc6c6ccccc56)cc4)c4ccc5c(c4)oc4c6ccccc6ccc54)cc3)cccc2c1]

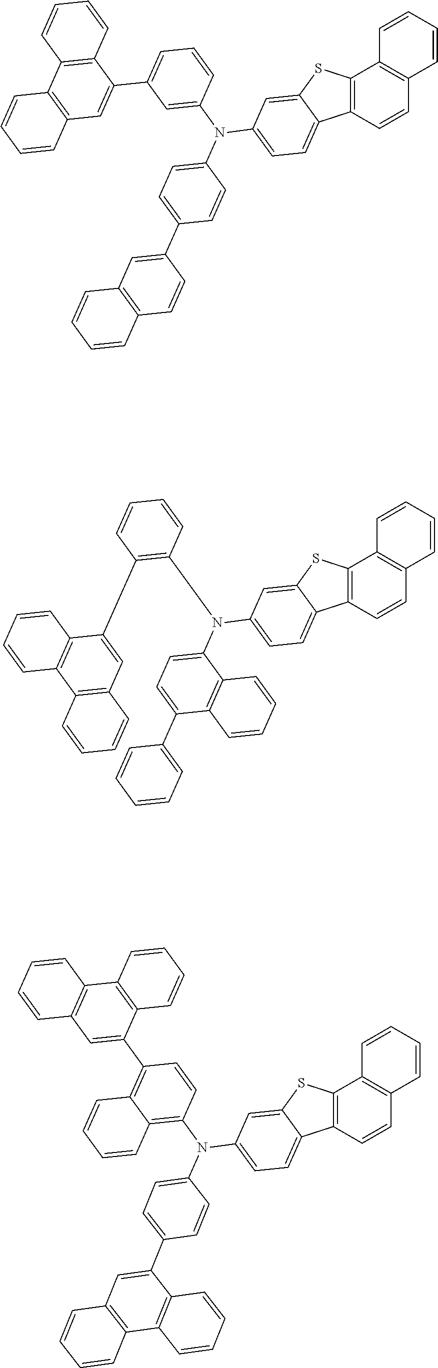 [SMILES: c1cc(-c2cc3ccccc3c3ccccc23)cc(N(c2ccc(-c3ccc4ccccc4c3)cc2)c2ccc3c(c2)sc2c4ccccc4ccc32)c1 ; c1ccc(-c2ccc(N(c3ccc4c(c3)sc3c5ccccc5ccc43)c3ccccc3-c3cc4ccccc4c4ccccc34)c3ccccc23)cc1 ; c1ccc2c(c1)cc(-c1ccc(N(c3ccc4c(c3)sc3c5ccccc5ccc43)c3ccc(-c4cc5ccccc5c5ccccc45)c4ccccc34)cc1)c1ccccc12]

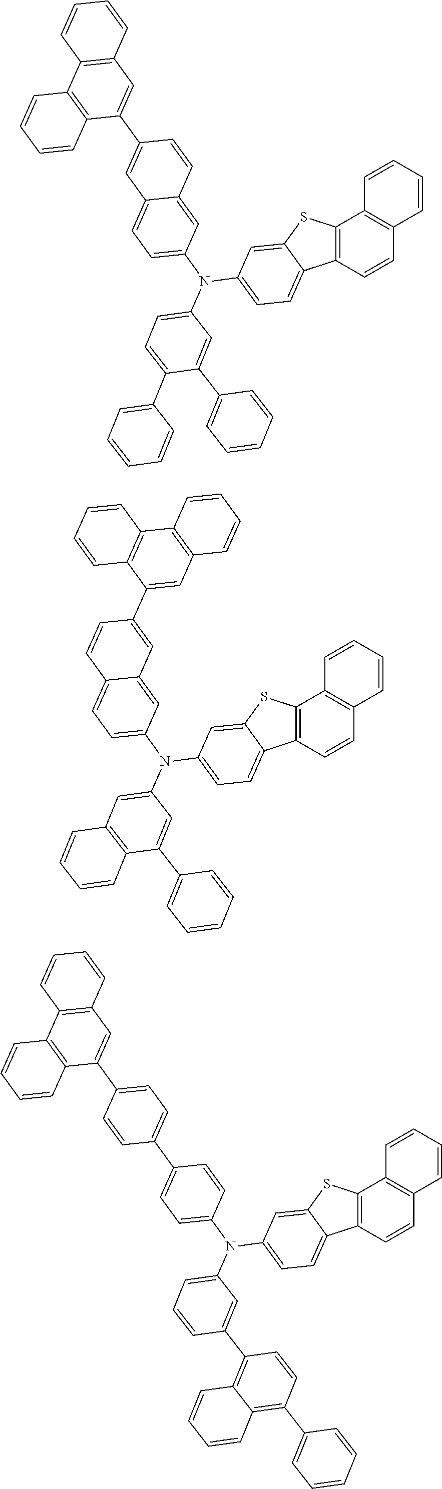

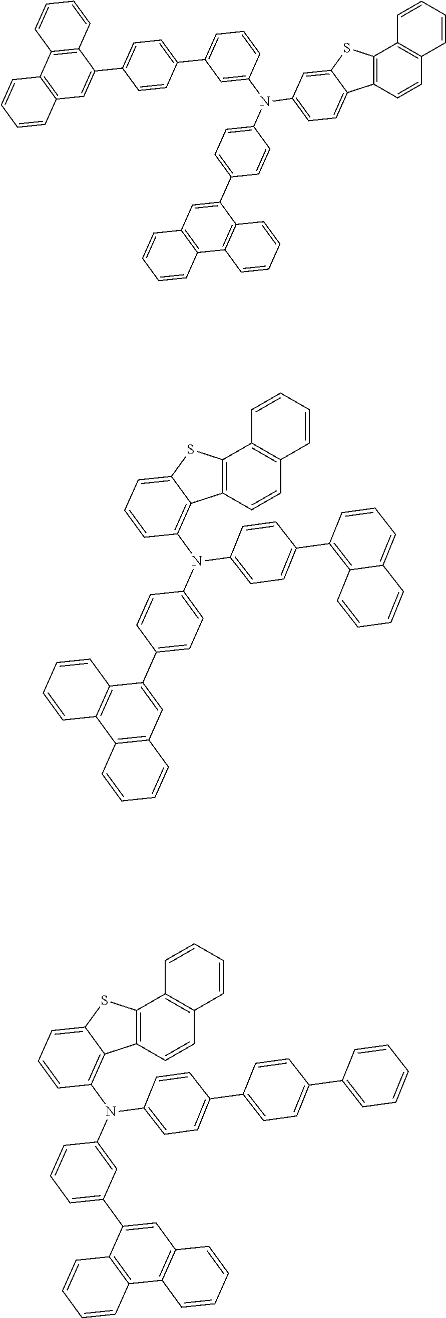 [SMILES: c1cc(-c2ccc(-c3cc4ccccc4c4ccccc34)cc2)cc(N(c2ccc(-c3cc4ccccc4c4ccccc34)cc2)c2ccc3c(c2)sc2c4ccccc4ccc32)c1 ; c1ccc2c(-c3ccc(N(c4ccc(-c5cc6ccccc6c6ccccc56)cc4)c4cccc5sc6c7ccccc7ccc6c45)cc3)cccc2c1 ; c1ccc(-c2ccc(-c3ccc(N(c4cccc(-c5cc6ccccc6c6ccccc56)c4)c4cccc5sc6c7ccccc7ccc6c45)cc3)cc2)cc1]

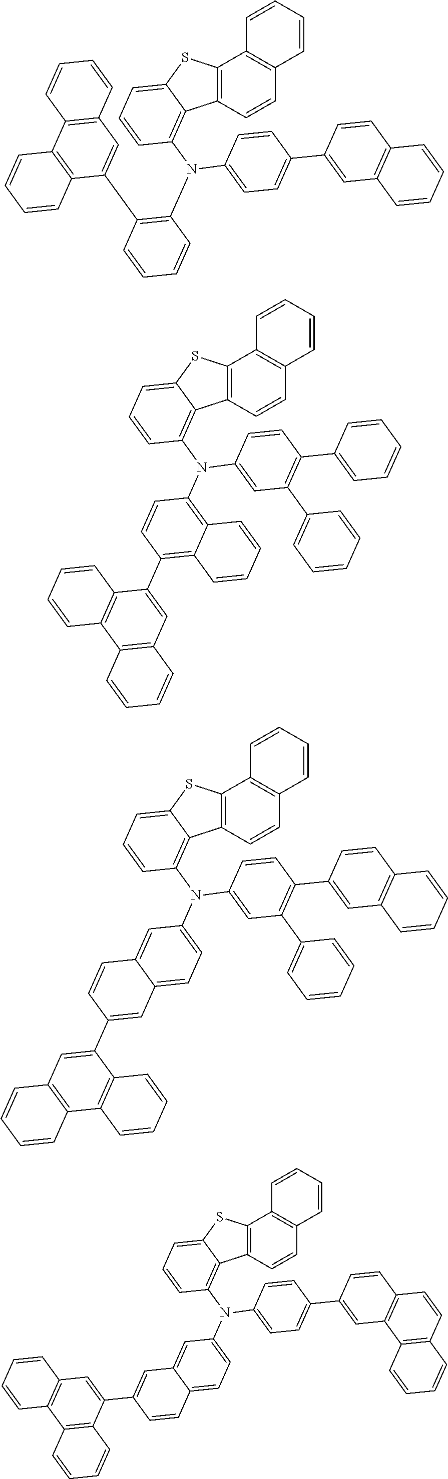 [SMILES: c1ccc(N(c2ccc(-c3ccc4ccccc4c3)cc2)c2cccc3sc4c5ccccc5ccc4c23)c(-c2cc3ccccc3c3ccccc23)c1 ; c1ccc(-c2ccc(N(c3ccc(-c4cc5ccccc5c5ccccc45)c4ccccc34)c3cccc4sc5c6ccccc6ccc5c34)cc2-c2ccccc2)cc1 ; c1ccc(-c2cc(N(c3ccc4cc(-c5cc6ccccc6c6ccccc56)ccc4c3)c3cccc4sc5c6ccccc6ccc5c34)ccc2-c2ccc3ccccc3c2)cc1 ; c1ccc2c(c1)ccc1ccc(-c3ccc(N(c4ccc5ccc(-c6cc7ccccc7c7ccccc67)cc5c4)c4cccc5sc6c7ccccc7ccc6c45)cc3)cc12]

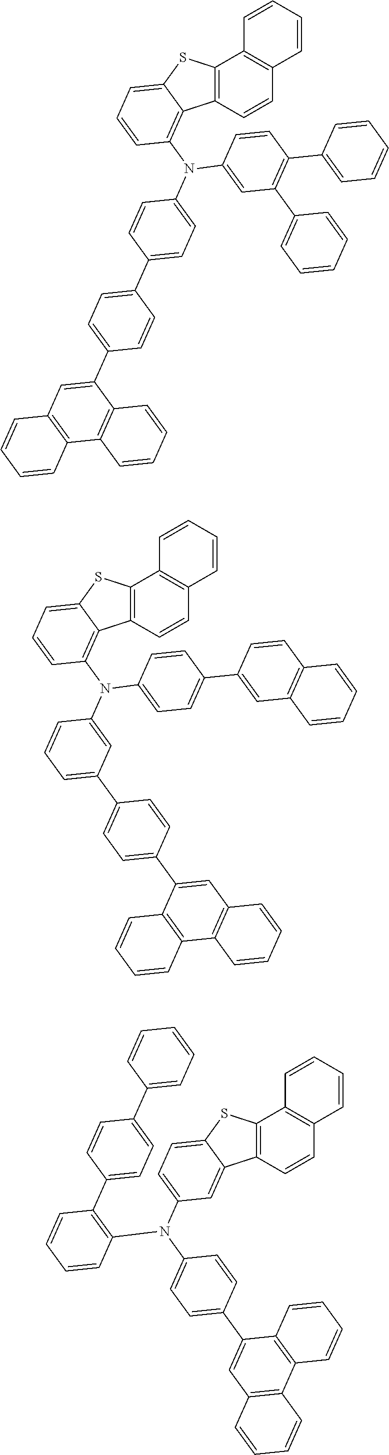 [SMILES: c1ccc(-c2ccc(N(c3ccc(-c4ccc(-c5cc6ccccc6c6ccccc56)cc4)cc3)c3cccc4sc5c6ccccc6ccc5c34)cc2-c2ccccc2)cc1 ; c1cc(-c2ccc(-c3cc4ccccc4c4ccccc34)cc2)cc(N(c2ccc(-c3ccc4ccccc4c3)cc2)c2cccc3sc4c5ccccc5ccc4c23)c1 ; c1ccc(-c2ccc(-c3ccccc3N(c3ccc(-c4cc5ccccc5c5ccccc45)cc3)c3ccc4sc5c6ccccc6ccc5c4c3)cc2)cc1]

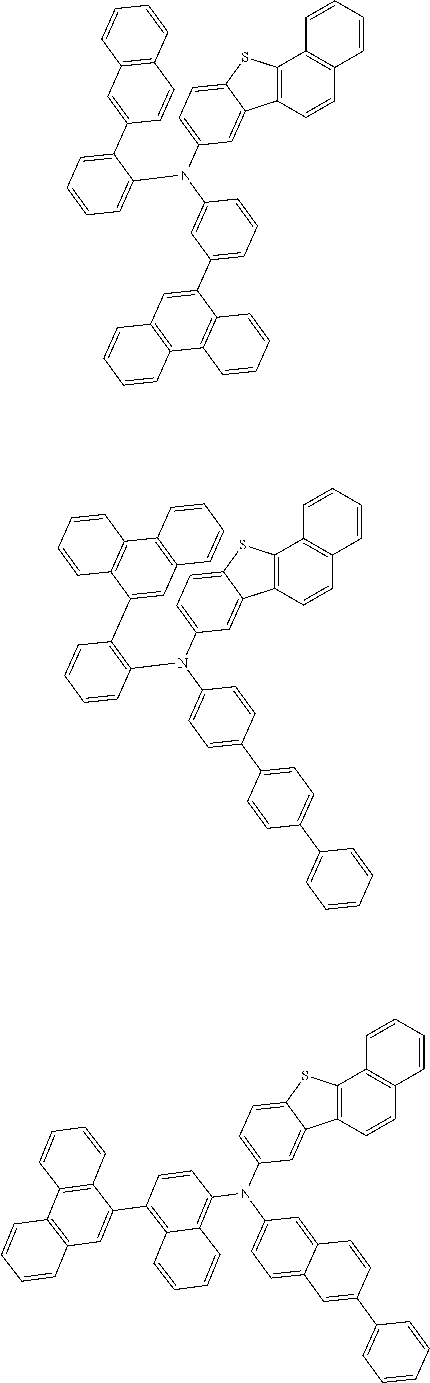 [SMILES: c1cc(-c2cc3ccccc3c3ccccc23)cc(N(c2ccc3sc4c5ccccc5ccc4c3c2)c2ccccc2-c2ccc3ccccc3c2)c1 ; c1ccc(-c2ccc(-c3ccc(N(c4ccc5sc6c7ccccc7ccc6c5c4)c4ccccc4-c4cc5ccccc5c5ccccc45)cc3)cc2)cc1 ; c1ccc(-c2ccc3cc(N(c4ccc5sc6c7ccccc7ccc6c5c4)c4ccc(-c5cc6ccccc6c6ccccc56)c5ccccc45)ccc3c2)cc1]

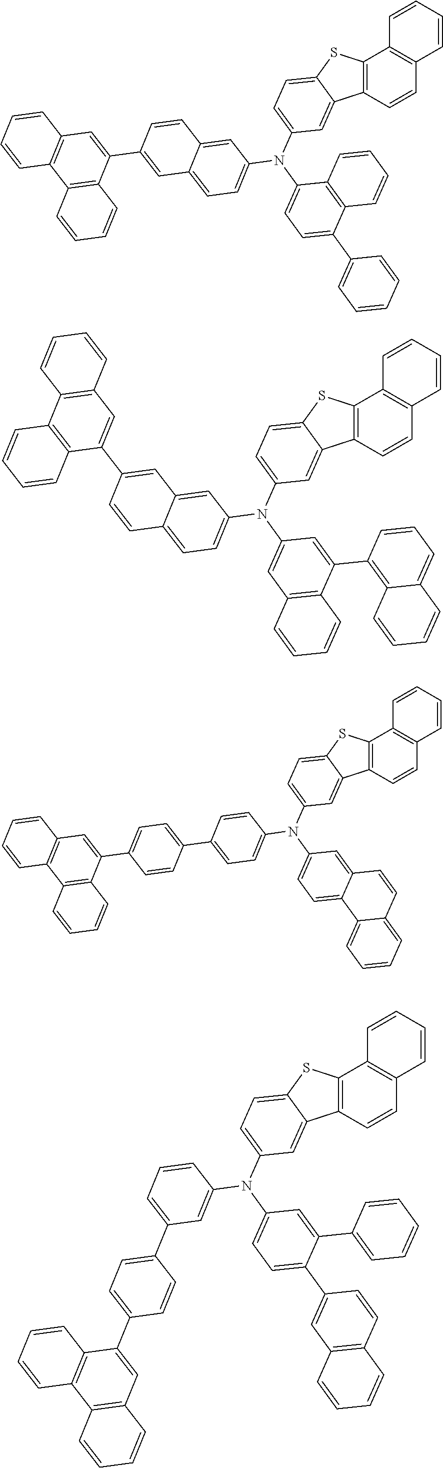

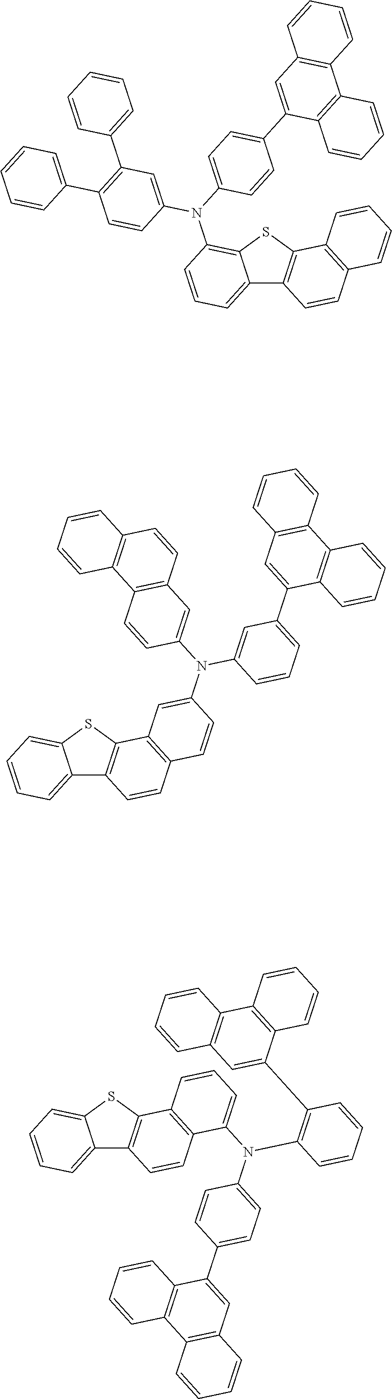 [SMILES: c1ccc(-c2ccc(N(c3ccc(-c4cc5ccccc5c5ccccc45)cc3)c3cccc4c3sc3c5ccccc5ccc43)cc2-c2ccccc2)cc1 ; c1cc(-c2cc3ccccc3c3ccccc23)cc(N(c2ccc3c(ccc4ccccc43)c2)c2ccc3ccc4c5ccccc5sc4c3c2)c1 ; c1ccc(N(c2ccc(-c3cc4ccccc4c4ccccc34)cc2)c2cccc3c2ccc2c4ccccc4sc32)c(-c2cc3ccccc3c3ccccc23)c1]

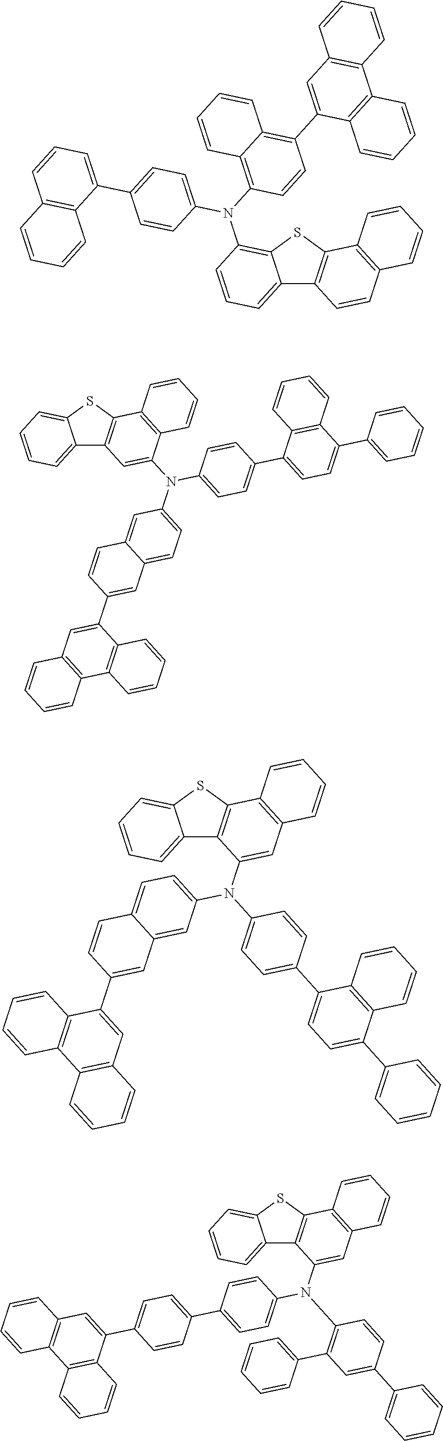 [SMILES: c1ccc2c(-c3ccc(N(c4ccc(-c5cc6ccccc6c6ccccc56)c5ccccc45)c4cccc5c4sc4c6ccccc6ccc54)cc3)cccc2c1 ; c1ccc(-c2ccc(-c3ccc(N(c4ccc5cc(-c6cc7ccccc7c7ccccc67)ccc5c4)c4cc5c6ccccc6sc5c5ccccc45)cc3)c3ccccc23)cc1 ; c1ccc(-c2ccc(-c3ccc(N(c4ccc5ccc(-c6cc7ccccc7c7ccccc67)cc5c4)c4cc5ccccc5c5sc6ccccc6c45)cc3)c3ccccc23)cc1 ; c1ccc(-c2ccc(N(c3ccc(-c4ccc(-c5cc6ccccc6c6ccccc56)cc4)cc3)c3cc4ccccc4c4sc5ccccc5c34)c(-c3ccccc3)c2)cc1]

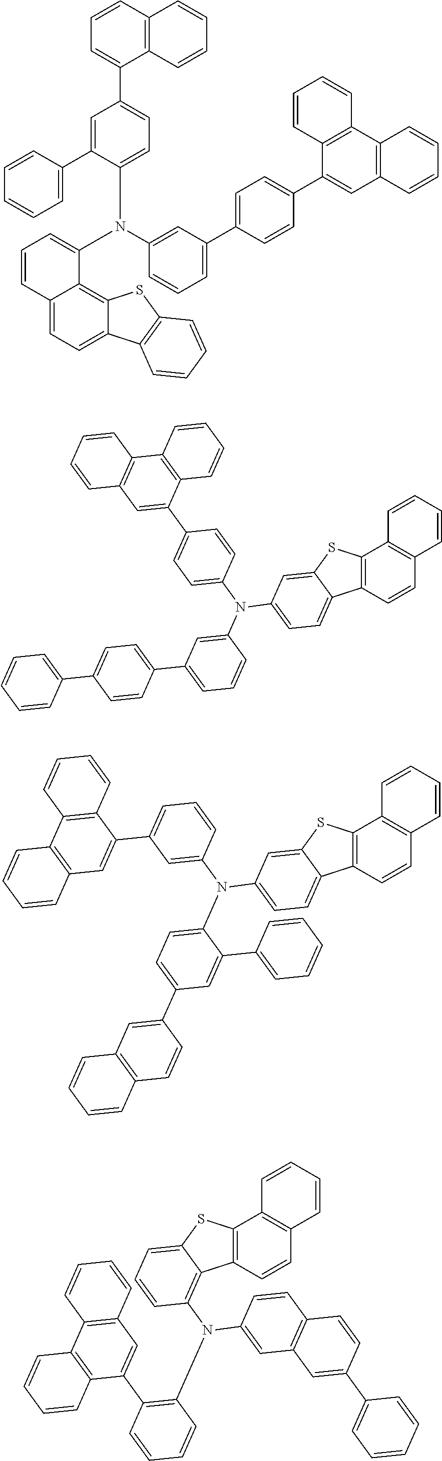 [SMILES: c1ccc(-c2cc(-c3cccc4ccccc34)ccc2N(c2cccc(-c3ccc(-c4cc5ccccc5c5ccccc45)cc3)c2)c2cccc3ccc4c5ccccc5sc4c23)cc1 ; c1ccc(-c2ccc(-c3cccc(N(c4ccc(-c5cc6ccccc6c6ccccc56)cc4)c4ccc5c(c4)sc4c6ccccc6ccc54)c3)cc2)cc1 ; c1ccc(-c2cc(-c3ccc4ccccc4c3)ccc2N(c2cccc(-c3cc4ccccc4c4ccccc34)c2)c2ccc3c(c2)sc2c4ccccc4ccc32)cc1 ; c1ccc(-c2ccc3ccc(N(c4ccccc4-c4cc5ccccc5c5ccccc45)c4cccc5sc6c7ccccc7ccc6c45)cc3c2)cc1]

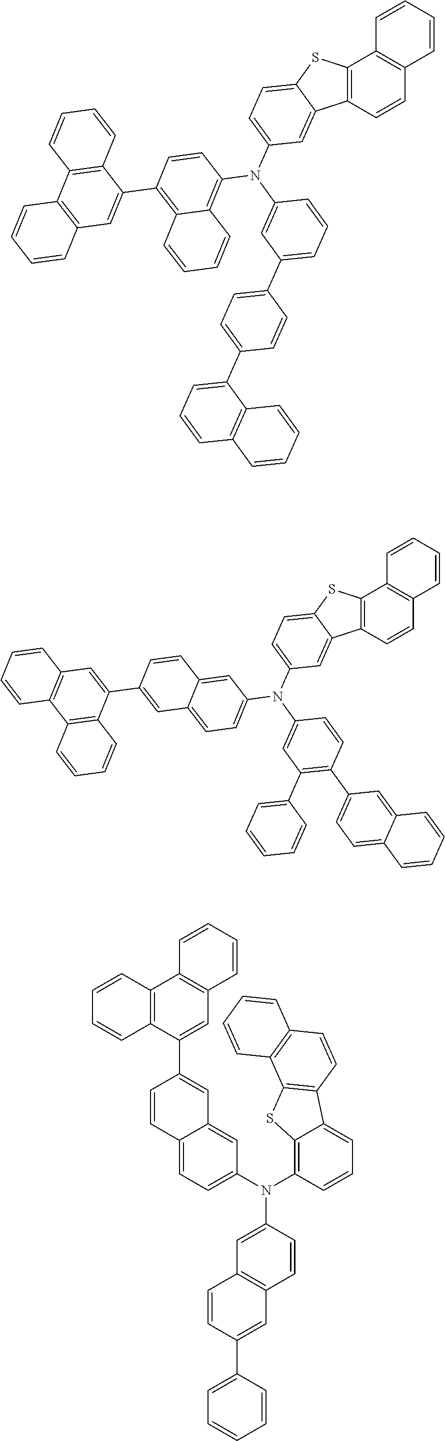 [SMILES: c1cc(-c2ccc(-c3cccc4ccccc34)cc2)cc(N(c2ccc3sc4c5ccccc5ccc4c3c2)c2ccc(-c3cc4ccccc4c4ccccc34)c3ccccc23)c1 ; c1ccc(-c2cc(N(c3ccc4cc(-c5cc6ccccc6c6ccccc56)ccc4c3)c3ccc4sc5c6ccccc6ccc5c4c3)ccc2-c2ccc3ccccc3c2)cc1 ; c1ccc(-c2ccc3cc(N(c4ccc5ccc(-c6cc7ccccc7c7ccccc67)cc5c4)c4cccc5c4sc4c6ccccc6ccc54)ccc3c2)cc1]

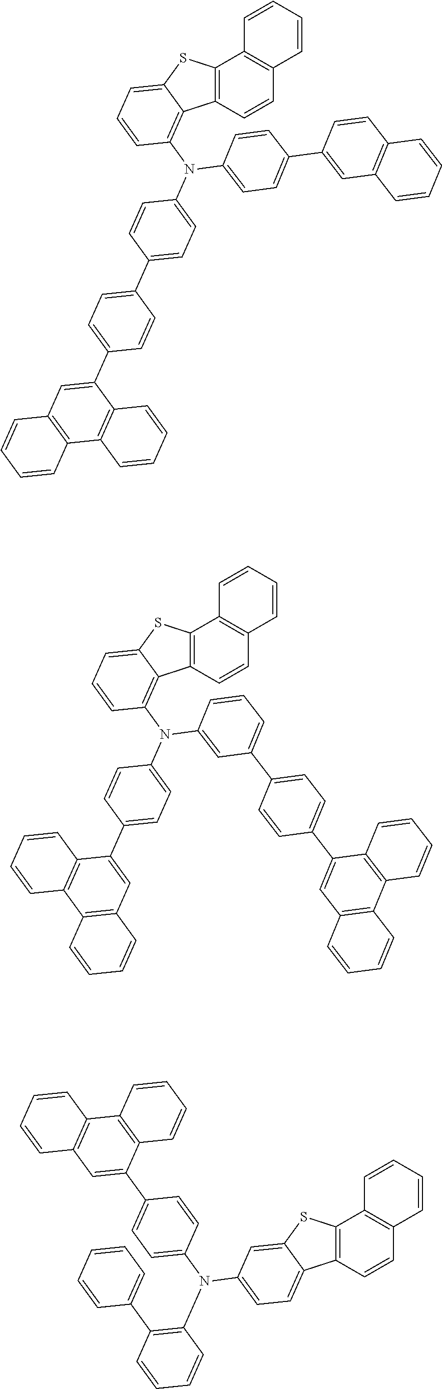 [SMILES: c1ccc2cc(-c3ccc(N(c4ccc(-c5ccc(-c6cc7ccccc7c7ccccc67)cc5)cc4)c4cccc5sc6c7ccccc7ccc6c45)cc3)ccc2c1 ; c1cc(-c2ccc(-c3cc4ccccc4c4ccccc34)cc2)cc(N(c2ccc(-c3cc4ccccc4c4ccccc34)cc2)c2cccc3sc4c5ccccc5ccc4c23)c1 ; c1ccc(-c2ccccc2N(c2ccc(-c3cc4ccccc4c4ccccc34)cc2)c2ccc3c(c2)sc2c4ccccc4ccc32)cc1]

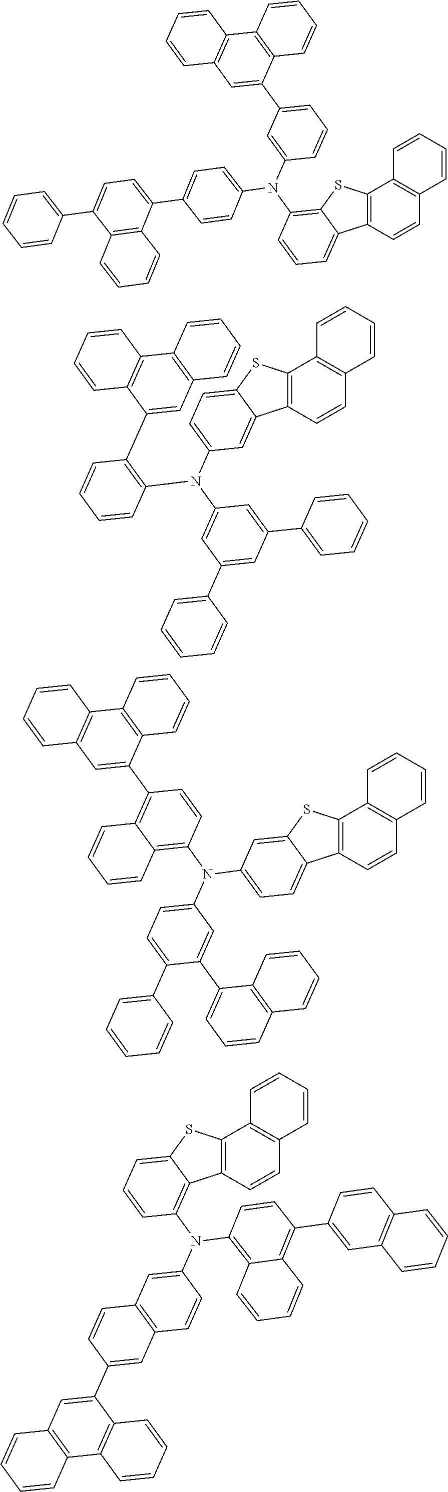

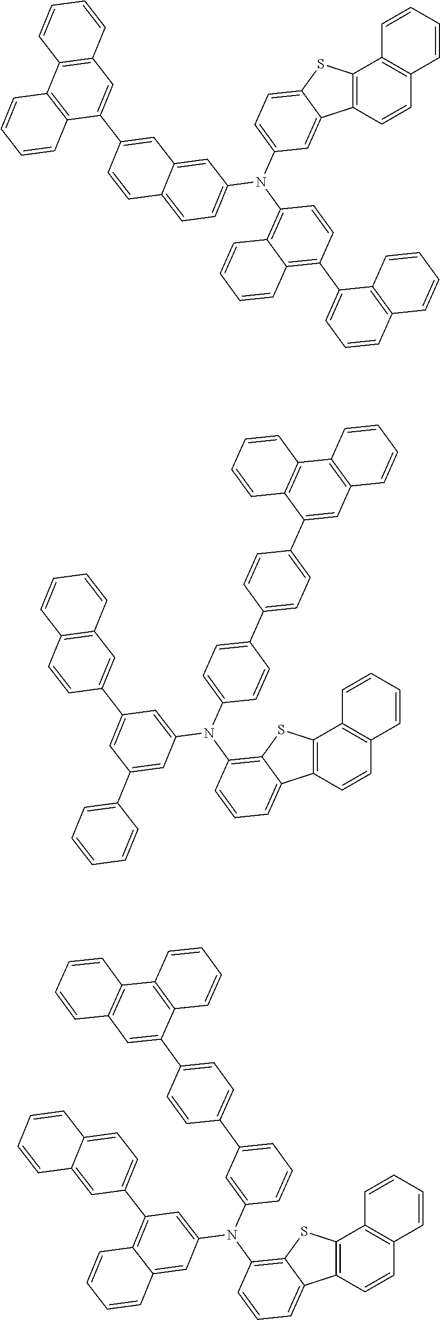 [SMILES: c1ccc2c(-c3ccc(N(c4ccc5ccc(-c6cc7ccccc7c7ccccc67)cc5c4)c4ccc5sc6c7ccccc7ccc6c5c4)c4ccccc34)cccc2c1 ; c1ccc(-c2cc(-c3ccc4ccccc4c3)cc(N(c3ccc(-c4ccc(-c5cc6ccccc6c6ccccc56)cc4)cc3)c3cccc4c3sc3c5ccccc5ccc43)c2)cc1 ; c1cc(-c2ccc(-c3cc4ccccc4c4ccccc34)cc2)cc(N(c2cc(-c3ccc4ccccc4c3)c3ccccc3c2)c2cccc3c2sc2c4ccccc4ccc32)c1]

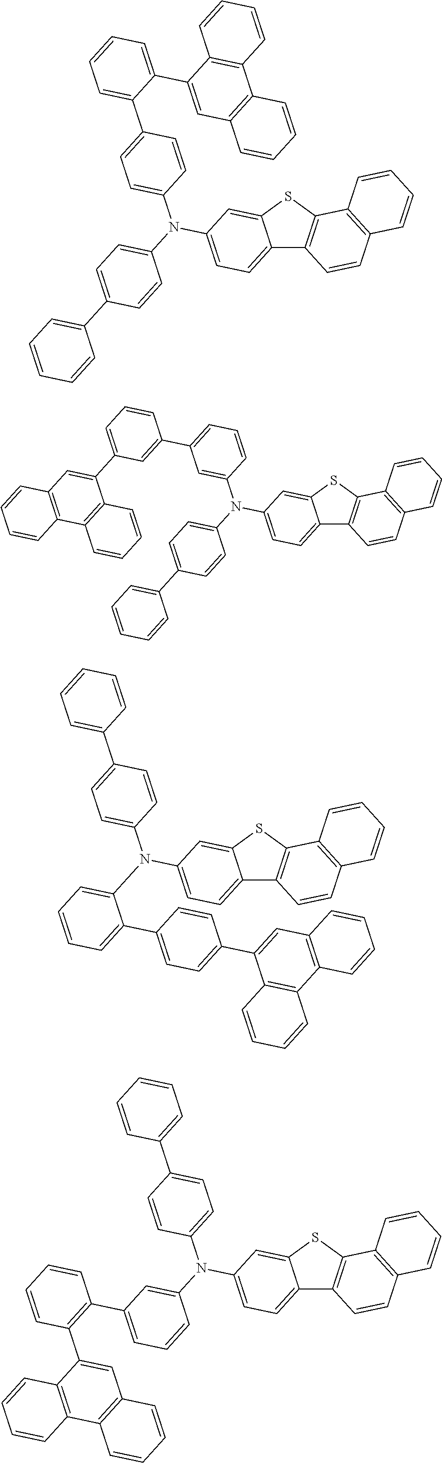

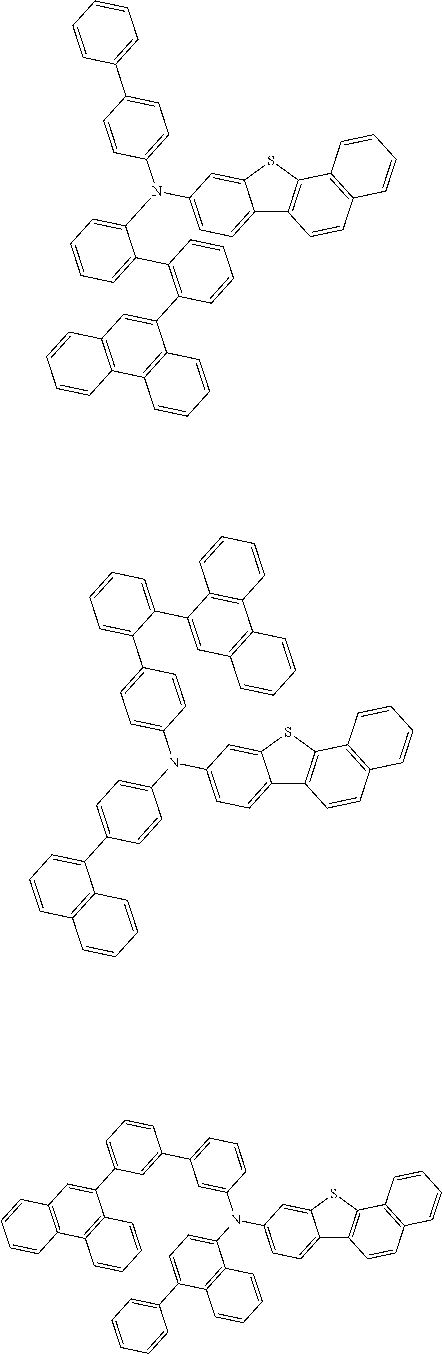 [SMILES: c1ccc(-c2ccc(N(c3ccc4c(c3)sc3c5ccccc5ccc43)c3ccccc3-c3ccccc3-c3cc4ccccc4c4ccccc34)cc2)cc1 ; c1ccc(-c2cc3ccccc3c3ccccc23)c(-c2ccc(N(c3ccc(-c4cccc5ccccc45)cc3)c3ccc4c(c3)sc3c5ccccc5ccc43)cc2)c1 ; c1ccc(-c2ccc(N(c3cccc(-c4cccc(-c5cc6ccccc6c6ccccc56)c4)c3)c3ccc4c(c3)sc3c5ccccc5ccc43)c3ccccc23)cc1]

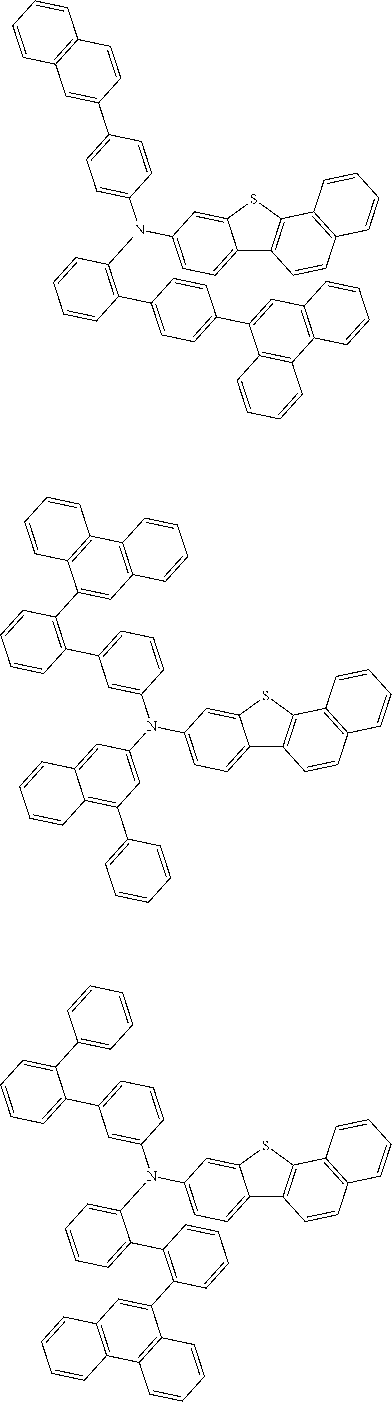 [SMILES: c1ccc(N(c2ccc(-c3ccc4ccccc4c3)cc2)c2ccc3c(c2)sc2c4ccccc4ccc32)c(-c2ccc(-c3cc4ccccc4c4ccccc34)cc2)c1 ; c1ccc(-c2cc(N(c3cccc(-c4ccccc4-c4cc5ccccc5c5ccccc45)c3)c3ccc4c(c3)sc3c5ccccc5ccc43)cc3ccccc23)cc1 ; c1ccc(-c2ccccc2-c2cccc(N(c3ccc4c(c3)sc3c5ccccc5ccc43)c3ccccc3-c3ccccc3-c3cc4ccccc4c4ccccc34)c2)cc1]

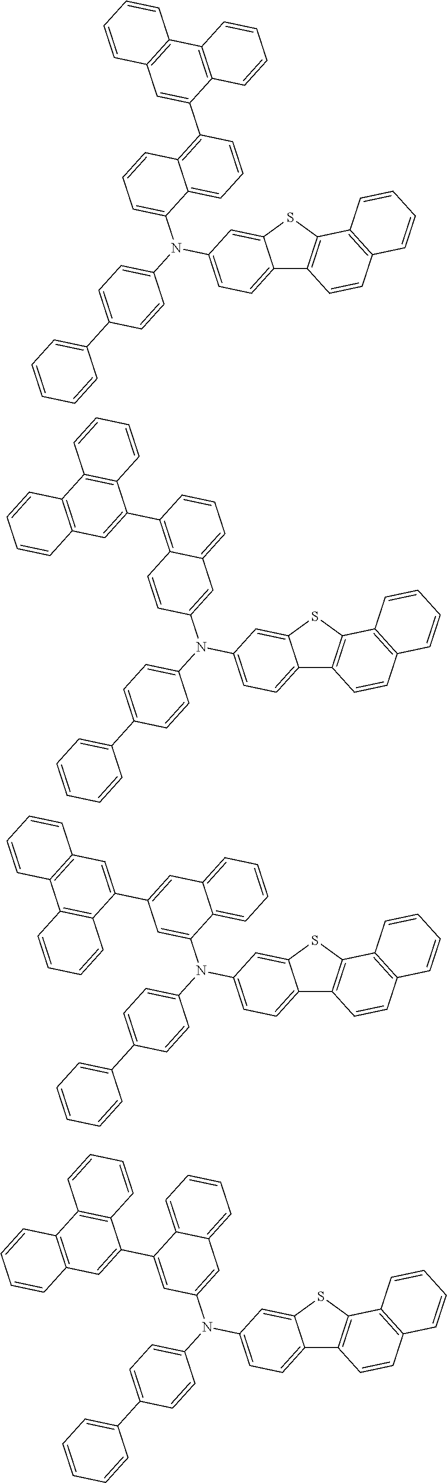

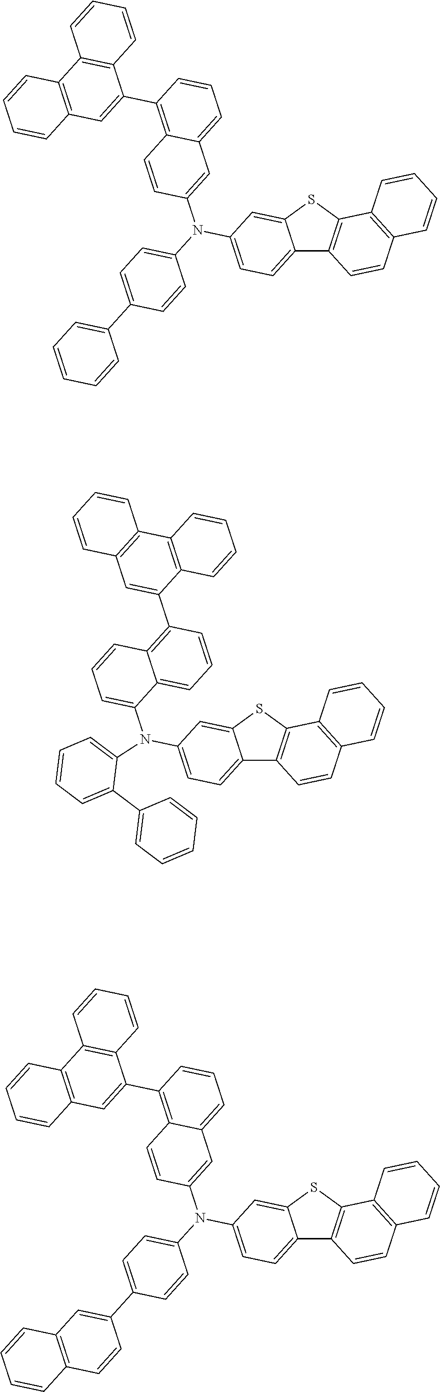 [SMILES: c1ccc(-c2ccc(N(c3ccc4c(-c5cc6ccccc6c6ccccc56)cccc4c3)c3ccc4c(c3)sc3c5ccccc5ccc43)cc2)cc1 ; c1ccc(-c2ccccc2N(c2ccc3c(c2)sc2c4ccccc4ccc32)c2cccc3c(-c4cc5ccccc5c5ccccc45)cccc23)cc1 ; c1ccc2cc(-c3ccc(N(c4ccc5c(-c6cc7ccccc7c7ccccc67)cccc5c4)c4ccc5c(c4)sc4c6ccccc6ccc54)cc3)ccc2c1]

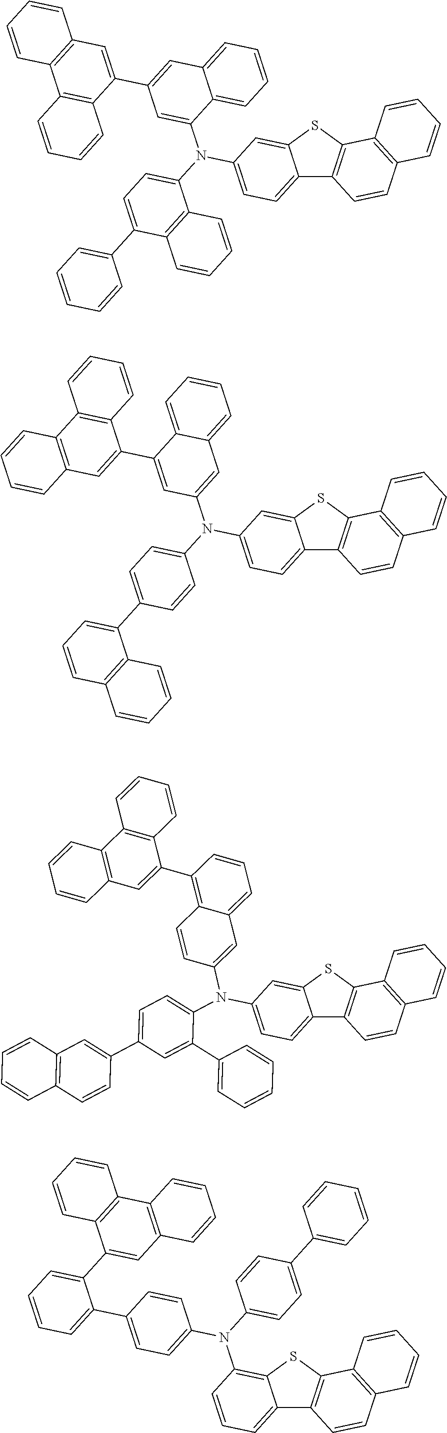

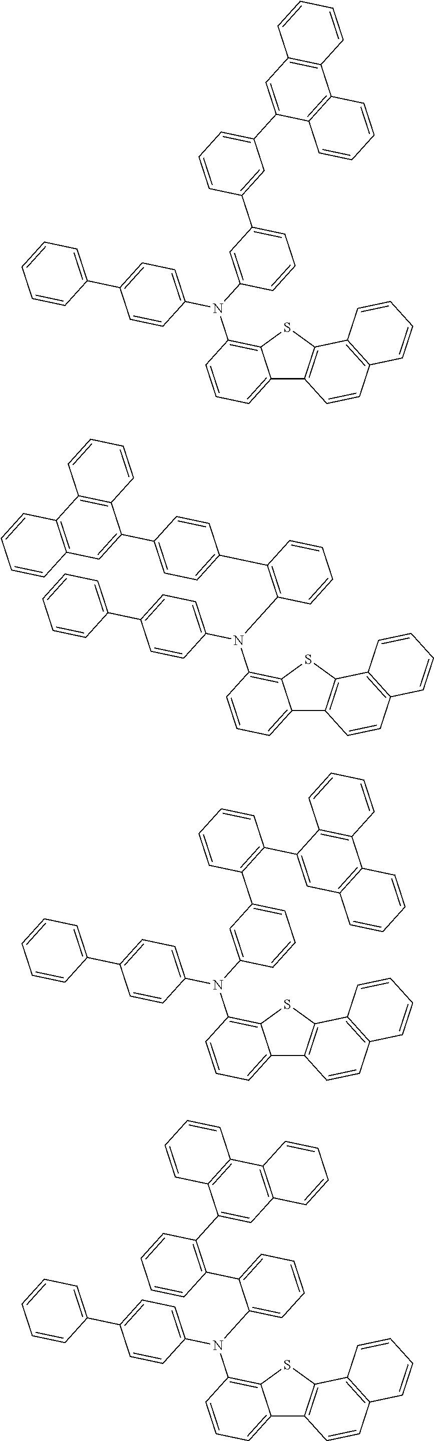

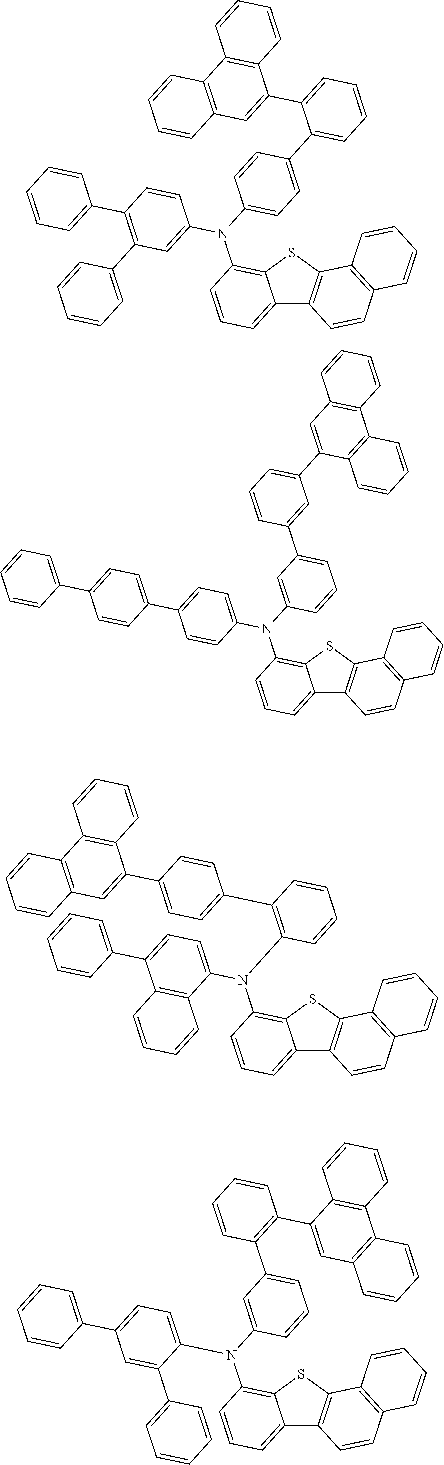

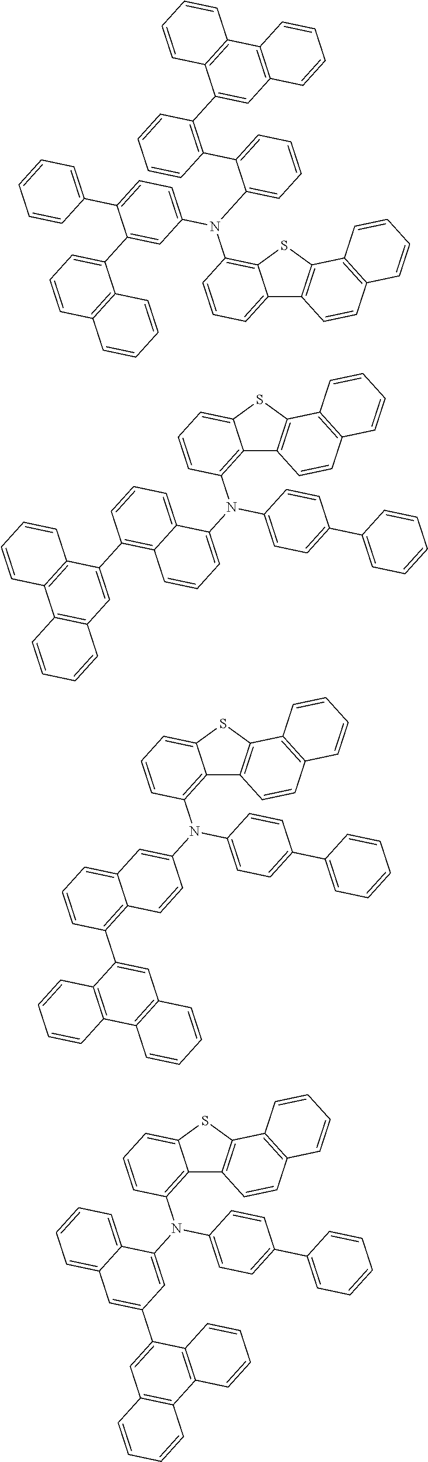 [SMILES: c1ccc(-c2ccc(N(c3ccccc3-c3ccccc3-c3cc4ccccc4c4ccccc34)c3cccc4c3sc3c5ccccc5ccc43)cc2-c2cccc3ccccc23)cc1 ; c1ccc(-c2ccc(N(c3cccc4c(-c5cc6ccccc6c6ccccc56)cccc34)c3cccc4sc5c6ccccc6ccc5c34)cc2)cc1 ; c1ccc(-c2ccc(N(c3ccc4c(-c5cc6ccccc6c6ccccc56)cccc4c3)c3cccc4sc5c6ccccc6ccc5c34)cc2)cc1 ; c1ccc(-c2ccc(N(c3cc(-c4cc5ccccc5c5ccccc45)cc4ccccc34)c3cccc4sc5c6ccccc6ccc5c34)cc2)cc1]

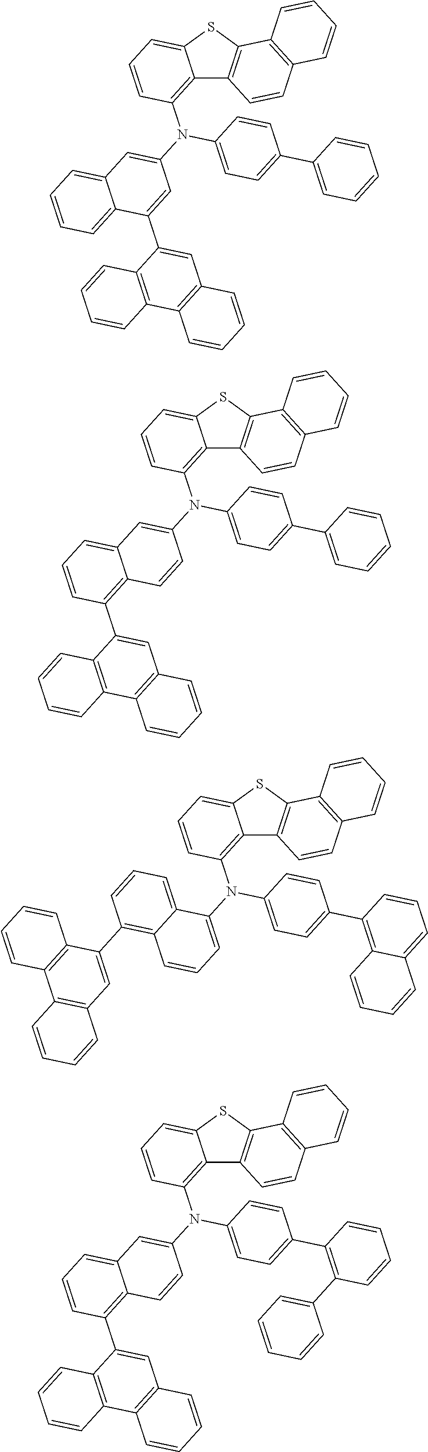 [SMILES: c1ccc(-c2ccc(N(c3cc(-c4cc5ccccc5c5ccccc45)c4ccccc4c3)c3cccc4sc5c6ccccc6ccc5c34)cc2)cc1 ; c1ccc(-c2ccc(N(c3ccc4c(-c5cc6ccccc6c6ccccc56)cccc4c3)c3cccc4sc5c6ccccc6ccc5c34)cc2)cc1 ; c1ccc2c(-c3ccc(N(c4cccc5c(-c6cc7ccccc7c7ccccc67)cccc45)c4cccc5sc6c7ccccc7ccc6c45)cc3)cccc2c1 ; c1ccc(-c2ccccc2-c2ccc(N(c3ccc4c(-c5cc6ccccc6c6ccccc56)cccc4c3)c3cccc4sc5c6ccccc6ccc5c34)cc2)cc1]

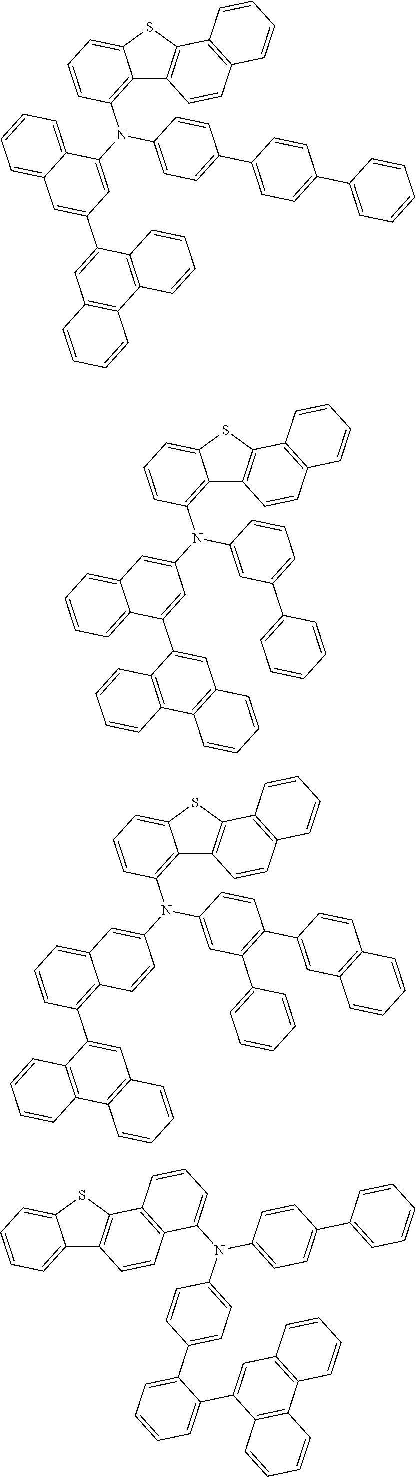

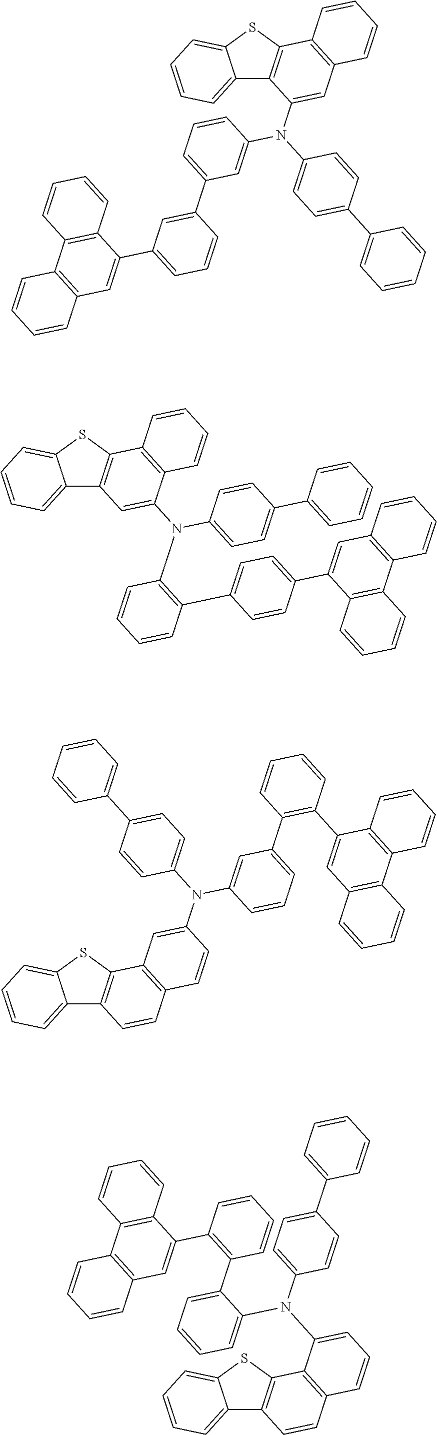

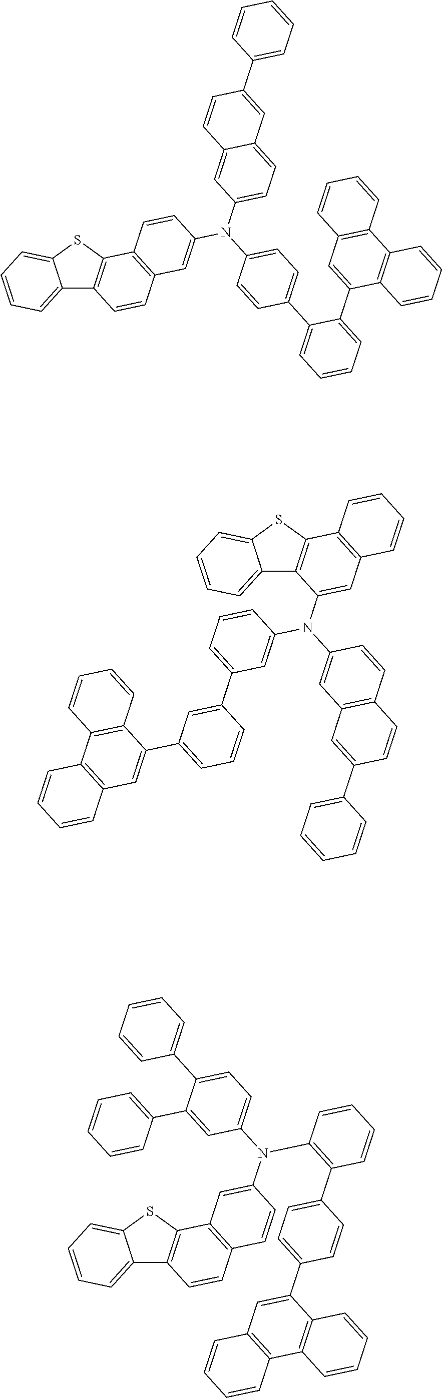

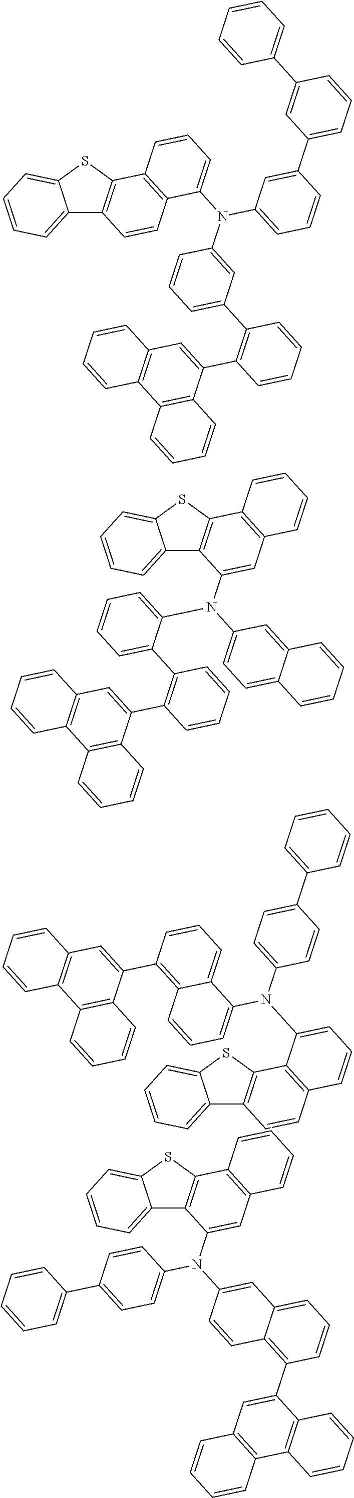

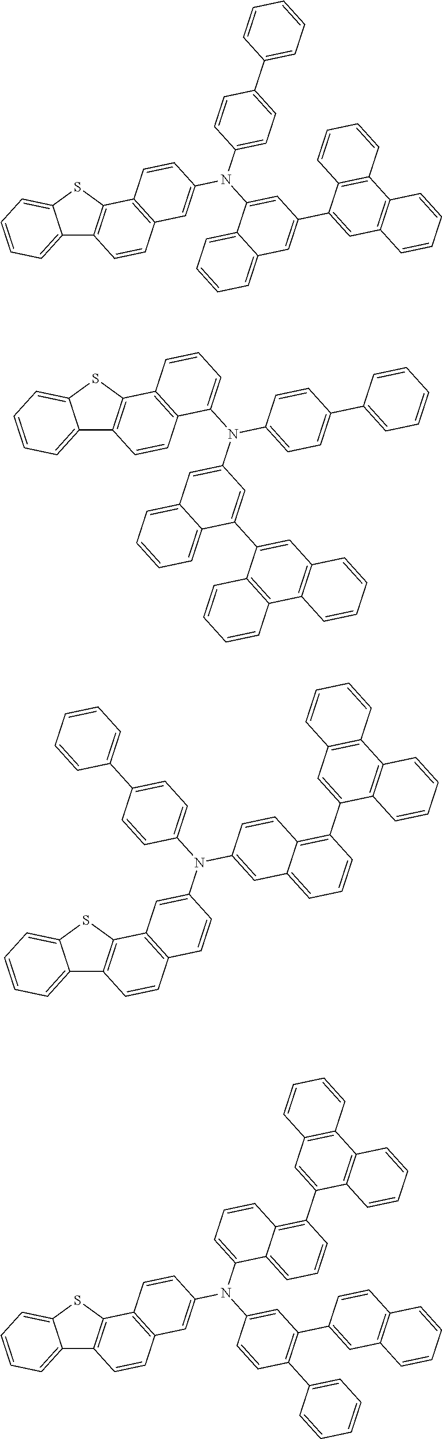 [SMILES: c1ccc(-c2ccc(N(c3ccc4c(ccc5c6ccccc6sc45)c3)c3cc(-c4cc5ccccc5c5ccccc45)cc4ccccc34)cc2)cc1 ; c1ccc(-c2ccc(N(c3cc(-c4cc5ccccc5c5ccccc45)c4ccccc4c3)c3cccc4c3ccc3c5ccccc5sc43)cc2)cc1 ; c1ccc(-c2ccc(N(c3ccc4c(-c5cc6ccccc6c6ccccc56)cccc4c3)c3ccc4ccc5c6ccccc6sc5c4c3)cc2)cc1 ; c1ccc(-c2ccc(N(c3ccc4c(ccc5c6ccccc6sc45)c3)c3cccc4c(-c5cc6ccccc6c6ccccc56)cccc34)cc2-c2ccc3ccccc3c2)cc1]

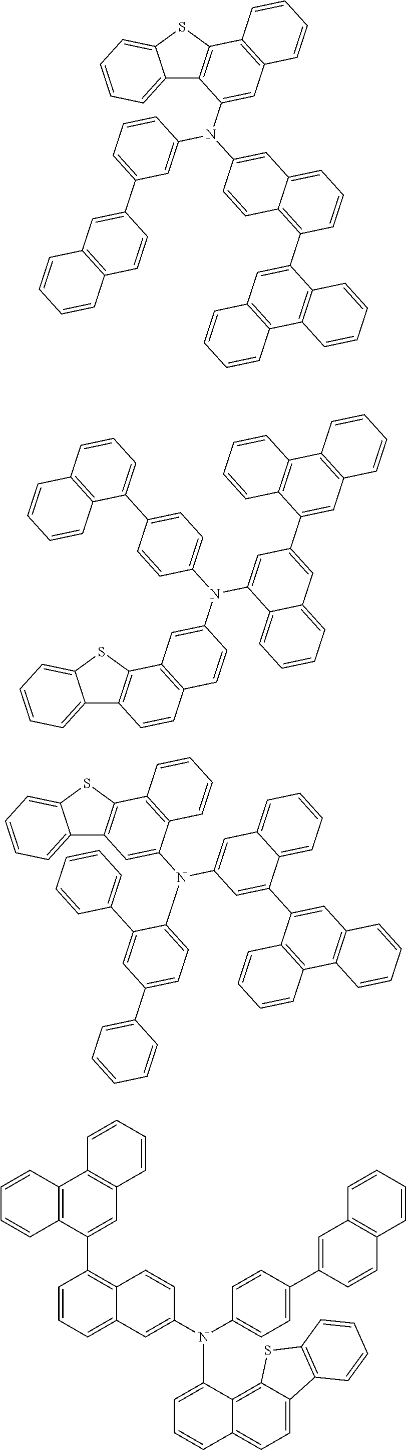

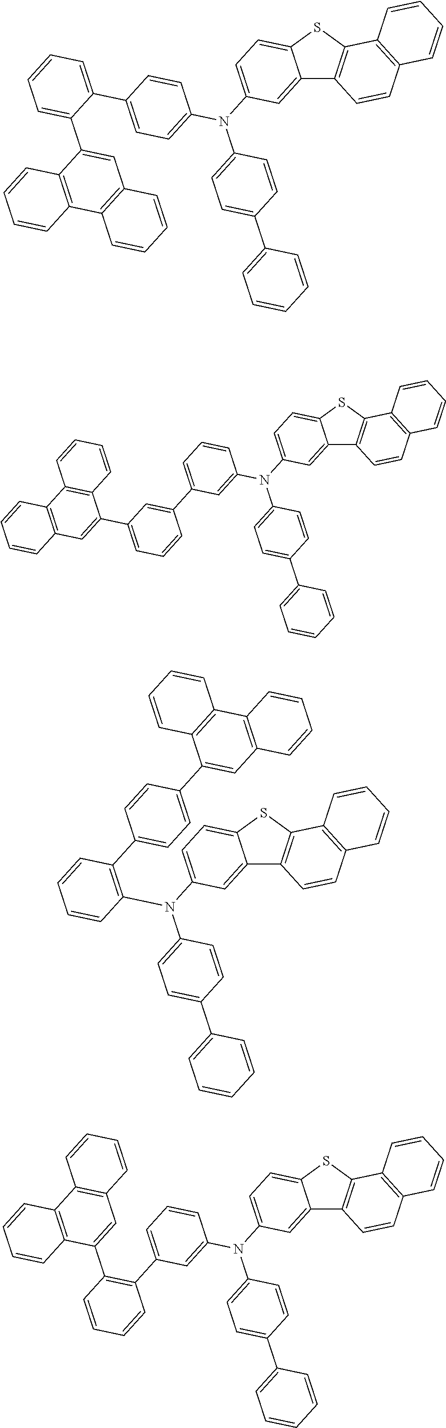 [SMILES: c1ccc(-c2ccc(N(c3ccc(-c4ccccc4-c4cc5ccccc5c5ccccc45)cc3)c3ccc4sc5c6ccccc6ccc5c4c3)cc2)cc1 ; c1ccc(-c2ccc(N(c3cccc(-c4cccc(-c5cc6ccccc6c6ccccc56)c4)c3)c3ccc4sc5c6ccccc6ccc5c4c3)cc2)cc1 ; c1ccc(-c2ccc(N(c3ccc4sc5c6ccccc6ccc5c4c3)c3ccccc3-c3ccc(-c4cc5ccccc5c5ccccc45)cc3)cc2)cc1 ; c1ccc(-c2ccc(N(c3cccc(-c4ccccc4-c4cc5ccccc5c5ccccc45)c3)c3ccc4sc5c6ccccc6ccc5c4c3)cc2)cc1]

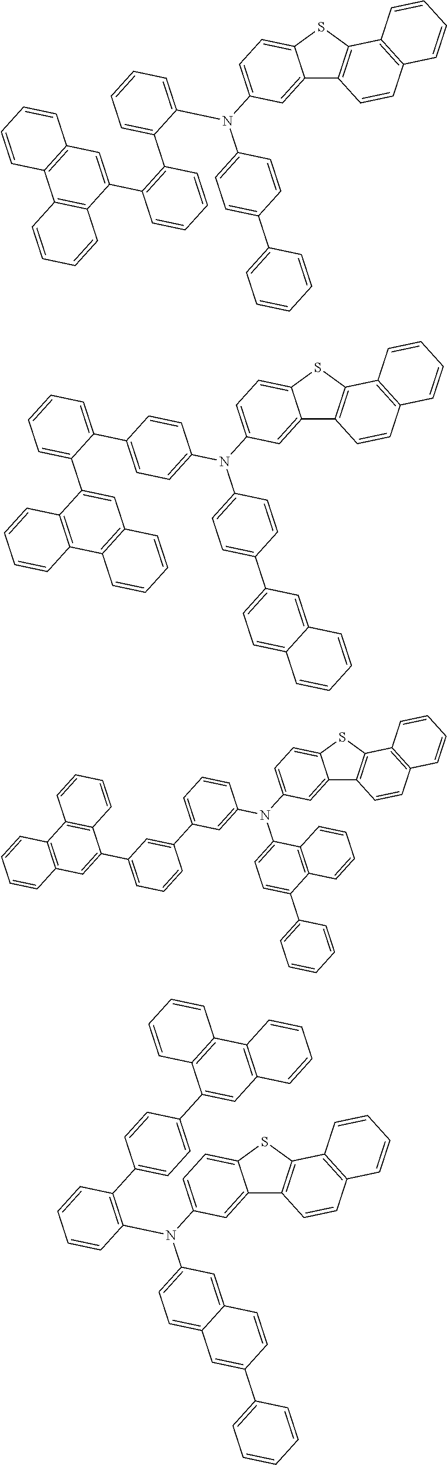 [SMILES: c1ccc(-c2ccc(N(c3ccc4sc5c6ccccc6ccc5c4c3)c3ccccc3-c3ccccc3-c3cc4ccccc4c4ccccc34)cc2)cc1 ; c1ccc(-c2cc3ccccc3c3ccccc23)c(-c2ccc(N(c3ccc(-c4ccc5ccccc5c4)cc3)c3ccc4sc5c6ccccc6ccc5c4c3)cc2)c1 ; c1ccc(-c2ccc(N(c3cccc(-c4cccc(-c5cc6ccccc6c6ccccc56)c4)c3)c3ccc4sc5c6ccccc6ccc5c4c3)c3ccccc23)cc1 ; c1ccc(-c2ccc3cc(N(c4ccc5sc6c7ccccc7ccc6c5c4)c4ccccc4-c4ccc(-c5cc6ccccc6c6ccccc56)cc4)ccc3c2)cc1]

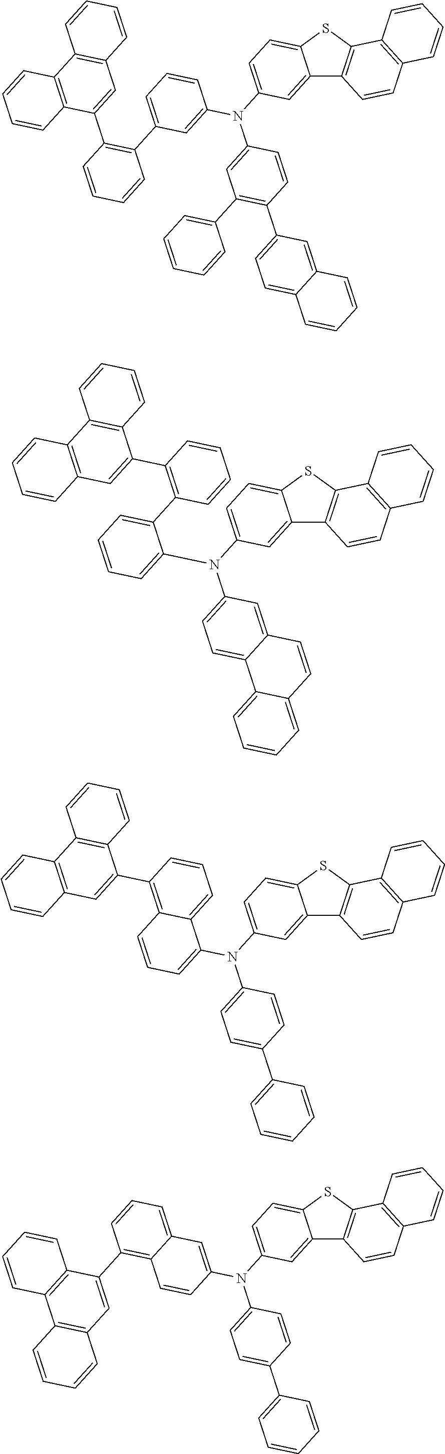 [SMILES: c1ccc(-c2cc(N(c3cccc(-c4ccccc4-c4cc5ccccc5c5ccccc45)c3)c3ccc4sc5c6ccccc6ccc5c4c3)ccc2-c2ccc3ccccc3c2)cc1 ; c1ccc(-c2cc3ccccc3c3ccccc23)c(-c2ccccc2N(c2ccc3c(ccc4ccccc43)c2)c2ccc3sc4c5ccccc5ccc4c3c2)c1 ; c1ccc(-c2ccc(N(c3ccc4sc5c6ccccc6ccc5c4c3)c3cccc4c(-c5cc6ccccc6c6ccccc56)cccc34)cc2)cc1 ; c1ccc(-c2ccc(N(c3ccc4c(-c5cc6ccccc6c6ccccc56)cccc4c3)c3ccc4sc5c6ccccc6ccc5c4c3)cc2)cc1]

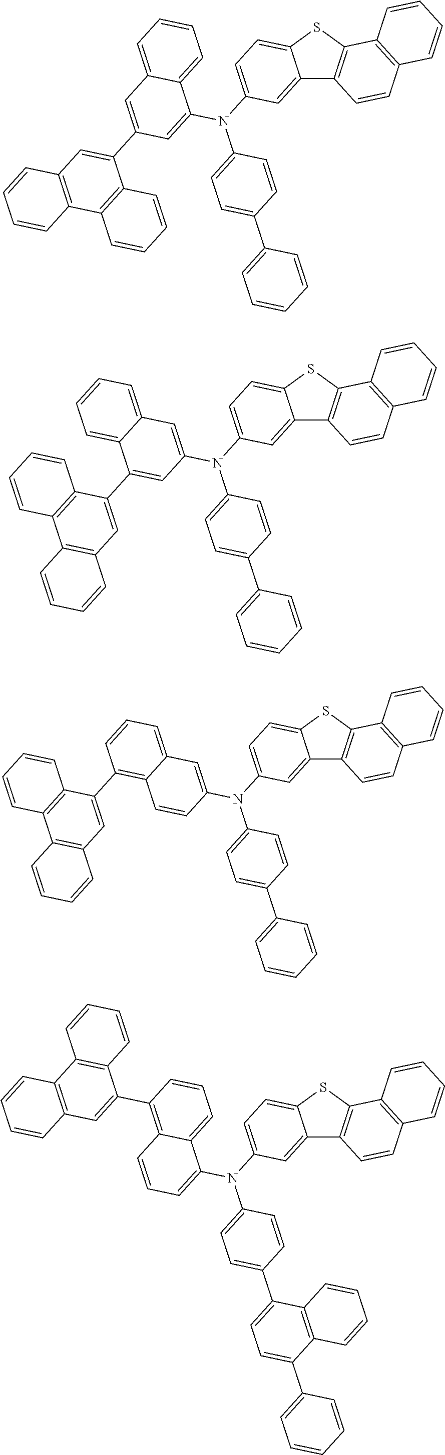 [SMILES: c1ccc(-c2ccc(N(c3ccc4sc5c6ccccc6ccc5c4c3)c3cc(-c4cc5ccccc5c5ccccc45)cc4ccccc34)cc2)cc1 ; c1ccc(-c2ccc(N(c3cc(-c4cc5ccccc5c5ccccc45)c4ccccc4c3)c3ccc4sc5c6ccccc6ccc5c4c3)cc2)cc1 ; c1ccc(-c2ccc(N(c3ccc4c(-c5cc6ccccc6c6ccccc56)cccc4c3)c3ccc4sc5c6ccccc6ccc5c4c3)cc2)cc1 ; c1ccc(-c2ccc(-c3ccc(N(c4ccc5sc6c7ccccc7ccc6c5c4)c4cccc5c(-c6cc7ccccc7c7ccccc67)cccc45)cc3)c3ccccc23)cc1]

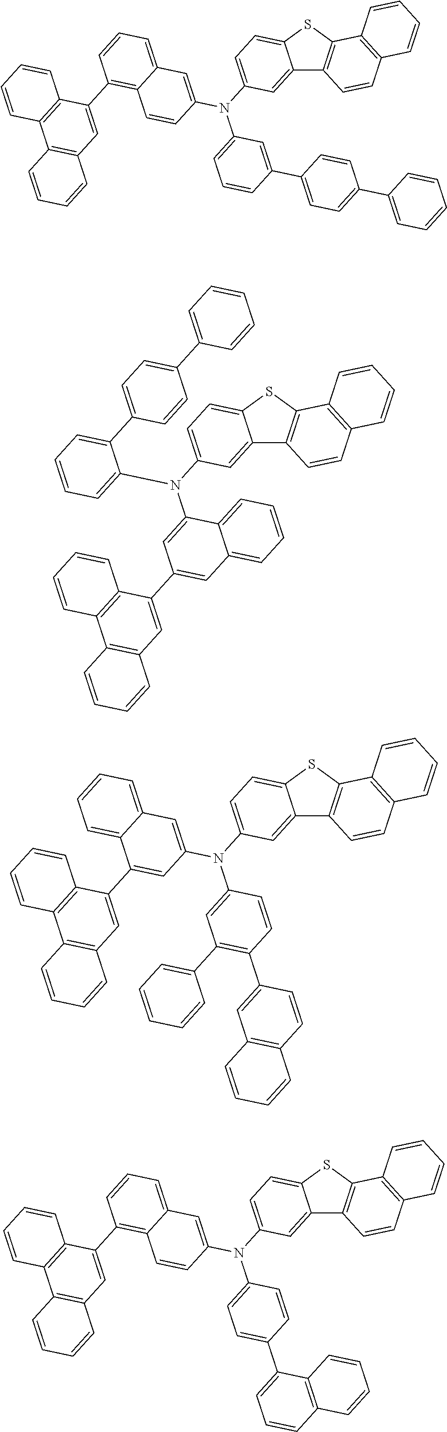 [SMILES: c1ccc(-c2ccc(-c3cccc(N(c4ccc5c(-c6cc7ccccc7c7ccccc67)cccc5c4)c4ccc5sc6c7ccccc7ccc6c5c4)c3)cc2)cc1 ; c1ccc(-c2ccc(-c3ccccc3N(c3ccc4sc5c6ccccc6ccc5c4c3)c3cc(-c4cc5ccccc5c5ccccc45)cc4ccccc34)cc2)cc1 ; c1ccc(-c2cc(N(c3cc(-c4cc5ccccc5c5ccccc45)c4ccccc4c3)c3ccc4sc5c6ccccc6ccc5c4c3)ccc2-c2ccc3ccccc3c2)cc1 ; c1ccc2c(-c3ccc(N(c4ccc5c(-c6cc7ccccc7c7ccccc67)cccc5c4)c4ccc5sc6c7ccccc7ccc6c5c4)cc3)cccc2c1]

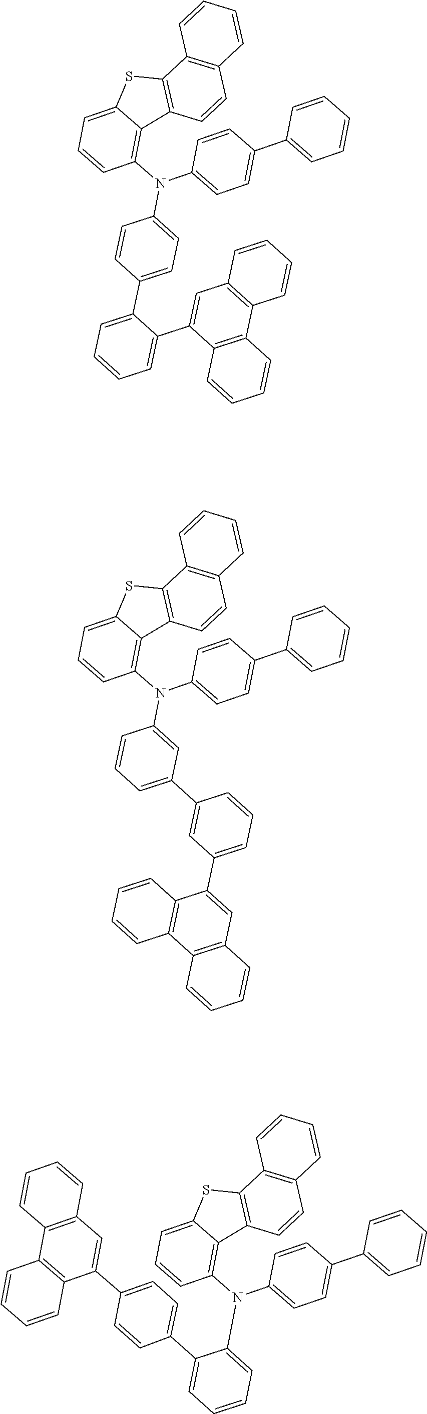 [SMILES: c1ccc(-c2ccc(N(c3ccc(-c4ccccc4-c4cc5ccccc5c5ccccc45)cc3)c3cccc4sc5c6ccccc6ccc5c34)cc2)cc1 ; c1ccc(-c2ccc(N(c3cccc(-c4cccc(-c5cc6ccccc6c6ccccc56)c4)c3)c3cccc4sc5c6ccccc6ccc5c34)cc2)cc1 ; c1ccc(-c2ccc(N(c3ccccc3-c3ccc(-c4cc5ccccc5c5ccccc45)cc3)c3cccc4sc5c6ccccc6ccc5c34)cc2)cc1]

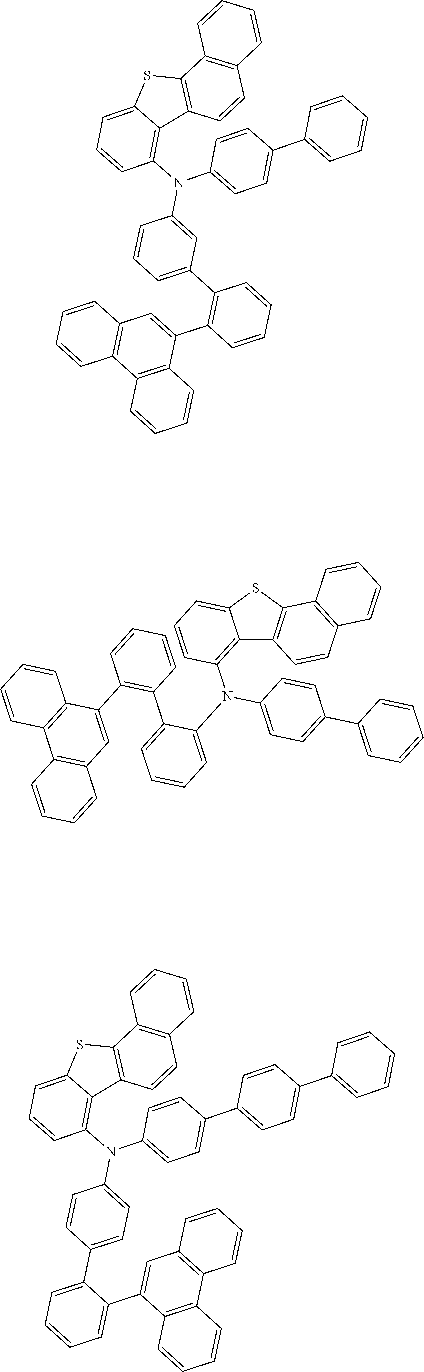 [SMILES: c1ccc(-c2ccc(N(c3cccc(-c4ccccc4-c4cc5ccccc5c5ccccc45)c3)c3cccc4sc5c6ccccc6ccc5c34)cc2)cc1 ; c1ccc(-c2ccc(N(c3ccccc3-c3ccccc3-c3cc4ccccc4c4ccccc34)c3cccc4sc5c6ccccc6ccc5c34)cc2)cc1 ; c1ccc(-c2ccc(-c3ccc(N(c4ccc(-c5ccccc5-c5cc6ccccc6c6ccccc56)cc4)c4cccc5sc6c7ccccc7ccc6c45)cc3)cc2)cc1]

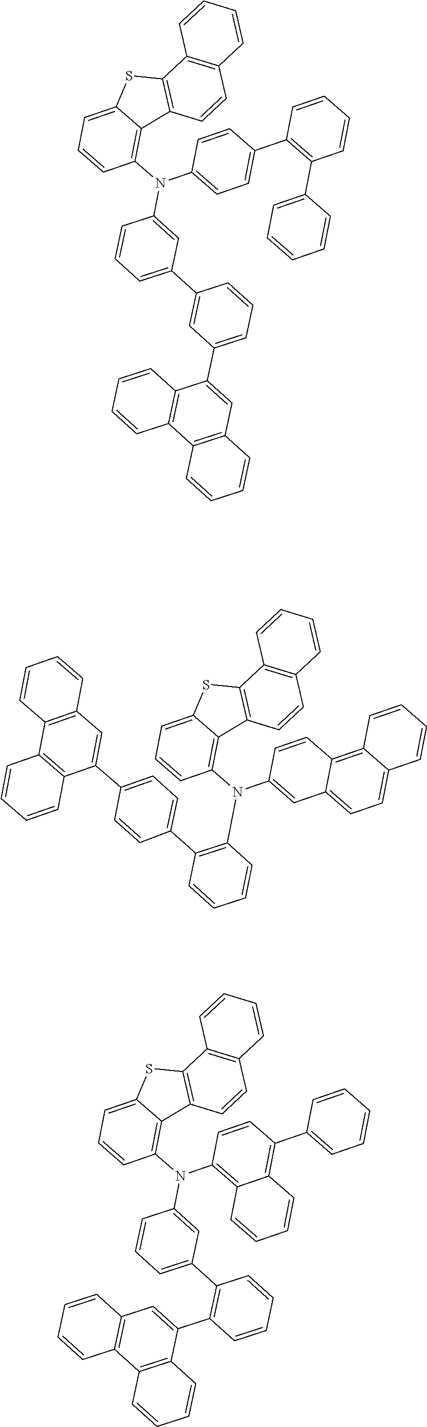 [SMILES: c1ccc(-c2ccccc2-c2ccc(N(c3cccc(-c4cccc(-c5cc6ccccc6c6ccccc56)c4)c3)c3cccc4sc5c6ccccc6ccc5c34)cc2)cc1 ; c1ccc(N(c2ccc3c(ccc4ccccc43)c2)c2cccc3sc4c5ccccc5ccc4c23)c(-c2ccc(-c3cc4ccccc4c4ccccc34)cc2)c1 ; c1ccc(-c2ccc(N(c3cccc(-c4ccccc4-c4cc5ccccc5c5ccccc45)c3)c3cccc4sc5c6ccccc6ccc5c34)c3ccccc23)cc1]

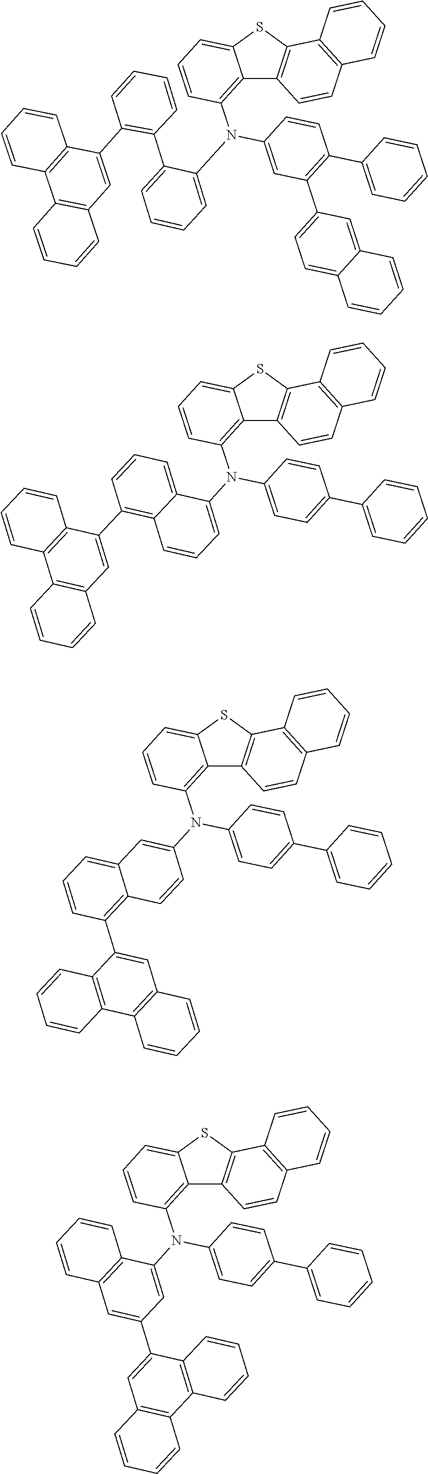

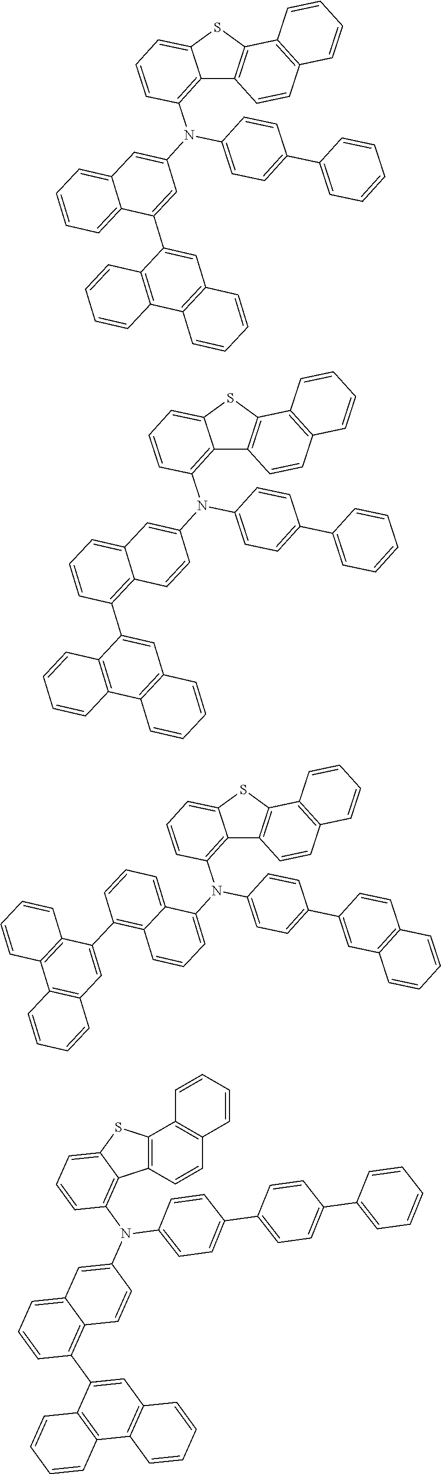 [SMILES: c1ccc(-c2ccc(N(c3cc(-c4cc5ccccc5c5ccccc45)c4ccccc4c3)c3cccc4sc5c6ccccc6ccc5c34)cc2)cc1 ; c1ccc(-c2ccc(N(c3ccc4c(-c5cc6ccccc6c6ccccc56)cccc4c3)c3cccc4sc5c6ccccc6ccc5c34)cc2)cc1 ; c1ccc2cc(-c3ccc(N(c4cccc5c(-c6cc7ccccc7c7ccccc67)cccc45)c4cccc5sc6c7ccccc7ccc6c45)cc3)ccc2c1 ; c1ccc(-c2ccc(-c3ccc(N(c4ccc5c(-c6cc7ccccc7c7ccccc67)cccc5c4)c4cccc5sc6c7ccccc7ccc6c45)cc3)cc2)cc1]

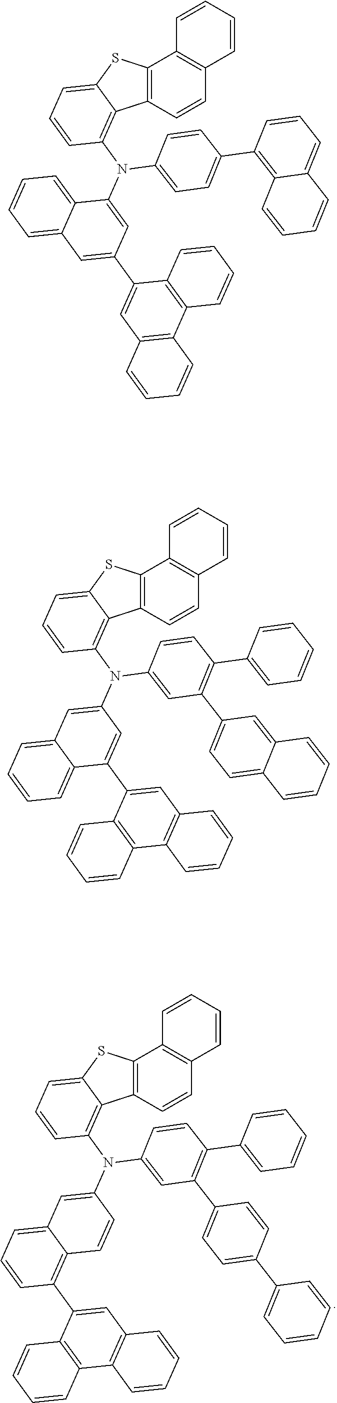 [SMILES: c1ccc2c(-c3ccc(N(c4cc(-c5cc6ccccc6c6ccccc56)cc5ccccc45)c4cccc5sc6c7ccccc7ccc6c45)cc3)cccc2c1 ; c1ccc(-c2ccc(N(c3cc(-c4cc5ccccc5c5ccccc45)c4ccccc4c3)c3cccc4sc5c6ccccc6ccc5c34)cc2-c2ccc3ccccc3c2)cc1 ; c1ccc(-c2ccc(-c3cc(N(c4ccc5c(-c6cc7ccccc7c7ccccc67)cccc5c4)c4cccc5sc6c7ccccc7ccc6c45)ccc3-c3ccccc3)cc2)cc1]

8. An organic light emitting device comprising:
a first electrode;
a second electrode that is provided opposite to the first electrode; and
one or more organic material layers that are provided between the first electrode and the second electrode, wherein one or more layers of the organic material layers comprises the compound of claim 1.

9. The organic light emitting device of claim 8, wherein the organic material layer comprising the compound is a light emitting layer.
